(19) United States
(12) Patent Application Publication
(10) Pub. No.: US 2009/0123928 A1
(43) Pub. Date: May 14, 2009

(54) Genomic Landscapes of Human Breast and Colorectal Cancers

(75) Inventors: Laura D. Wood, Baltimore, MD (US); Williams D. Parsons, Ellicott City, MD (US); Sian Jones, Baltimore, MD (US); Jimmy Lin, Baltimore, MD (US); Tobias Sjoblom, Uppsala (SE); Thomas Barber, Noblesville, IN (US); Giovanni Parmigiani, Baltimore, MD (US); Victor Velculescu, Dayton, MD (US); Kenneth W. Kinzler, Bel Air, MD (US); Bert Vogelstein, Baltimore, MD (US)

Correspondence Address: BANNER & WITCOFF, LTD., 1100 13th STREET, N.W., SUITE 1200, WASHINGTON, DC, 20005-4051, US

(73) Assignee: The Johns Hopkins University, Baltimore, MD (US)

(21) Appl. No.: 12/247,464

(22) Filed: Oct. 8, 2008

Related U.S. Application Data
(60) Provisional application No. 60/960,733, filed on Oct. 11, 2007.

(51) Int. Cl. C12Q 1/68; C12Q 1/02

(52) U.S. Cl. 435/6; 435/29

(57) ABSTRACT

Human cancer is caused by the accumulation of mutations in oncogenes and tumor suppressor genes. To catalogue the genetic changes that occur during tumorigenesis, we isolated DNA from 11 breast and 11 colorectal tumors and determined the sequences of the genes in the Reference Sequence database in these samples. Based on analysis of exons representing 20,857 transcripts from 18,191 genes, we conclude that the genomic landscapes of breast and colorectal cancers are composed of a handful of commonly mutated gene “mountains” and a much larger number of gene “hills” that are mutated at low frequency. We describe statistical and bioinformatic tools that may help identify mutations with a role in tumorigenesis. These results have implications for understanding the nature and heterogeneity of human cancers and for using personal genomics for tumor diagnosis and therapy.

This invention was made using grant funds from the U.S. government. Under the term of the grants, the U.S. government retains certain rights in the invention. Grants used include NIH grants CA 43460, CA 57345, CA 12113, and CA 62924.

A sequence listing is provided on a single compact disc. The compact disc contains a file named templst.txt. The file is 22695 kb and was created Oct. 3, 2008. The content of the compact disc is incorporated herein.

### TECHNICAL FIELD OF THE INVENTION

This invention is related to the area of cancer characterization. In particular, it relates to breast and colorectal cancers.

### BACKGROUND OF THE INVENTION

Discovery of the genes mutated in human cancer has provided key insights into the mechanisms underlying tumorigenesis and has proven useful for the design of a new generation of targeted approaches for clinical intervention (1). With the determination of the human genome sequence and improvements in sequencing and bioinformatic technologies, systematic analyses of genetic alterations in human cancers have become possible (2-4).

Using such large-scale approaches, we recently studied the genomes of breast and colorectal cancers by determining the sequence of the Consensus Coding Sequence (CCDS) genes, a collection of the best annotated protein coding genes (5). In the current study, we have extended these analyses to include examination of all of the Reference Sequence (RefSeq) genes. The RefSeq database is a comprehensive, non-redundant collection of annotated gene sequences that represents a consolidation of gene information from all major gene databases (6). The RefSeq database is believed to include the great majority of human gene sequences and represents the gold standard in the field.

There is a continuing need in the art to identify genes and patterns of gene mutations useful for identifying and stratifying individual patients' cancers.

### SUMMARY OF THE INVENTION

According to one embodiment of the invention a method is provided for diagnosing breast cancer in a human. A somatic mutation in a gene or its encoded cDNA or protein is determined in a test sample relative to a normal sample of the human. The gene is selected from the group consisting of those listed in FIG. 10 (Table S4B) The sample is identified as breast cancer when the somatic mutation is determined.

A method is provided for diagnosing colorectal cancer in a human. A somatic mutation in a gene or its encoded cDNA or protein is determined in a test sample relative to a normal sample of the human. The gene is selected from the group consisting of those listed in FIG. 9 (Table S4A). The sample is identified as colorectal cancer if the somatic mutation is determined.

A method is provided for stratifying breast cancers for testing candidate or known anti-cancer therapeutics. A CAN-gene mutational signature for a breast cancer is determined by determining at least one somatic mutation in a test sample relative to a normal sample of a human. The at least one somatic mutation is in one or more genes selected from the group consisting of FIG. 10 (Table S4B) A first group of breast cancers that have the CAN-gene mutational signature is formed. Efficacy of a candidate or known anti-cancer therapeutic on the first group is compared to efficacy on a second group of breast cancers that has a different CAN-gene mutational signature. A CAN gene mutational signature which correlates with increased or decreased efficacy of the candidate or known anti-cancer therapeutic relative to other groups is identified.

A method is provided for stratifying colorectal cancers for testing candidate or known anti-cancer therapeutics. A CAN-gene mutational signature for a colorectal cancer is determined by determining at least one somatic mutation in a test sample relative to a normal sample of the human. The at least one somatic mutation is in one or more genes selected from the group consisting of FIG. 9 (Table S4A). A first group of colorectal cancers that have the CAN-gene mutational signature is formed. Efficacy of a candidate or known anti-cancer therapeutic on the first group is compared to efficacy on a second group of colorectal cancers that has a different CAN-gene mutational signature. A CAN gene mutational signature is identified which correlates with increased or decreased efficacy of the candidate or known anti-cancer therapeutic relative to other groups.

A method is provided for characterizing a breast cancer in a human. A somatic mutation in a gene or its encoded cDNA or protein is determined in a test sample relative to a normal sample of the human. The gene is selected from the group consisting of those listed in FIG. 10 (Table S4B)

Another method provided is for characterizing a colorectal cancer in a human. A somatic mutation in a gene or its encoded cDNA or protein is determined in a test sample relative to a normal sample of the human. The gene is selected from the group consisting of those listed in FIG. 9 (Table S4A).

These and other embodiments which will be apparent to those of skill in the art upon reading the specification provide the art with additional methods and tools for better managing cancer treatment.

### BRIEF DESCRIPTION OF THE DRAWINGS

FIG. 1 Clustering of somatic mutations in protein structures. Individual somatic mutations were mapped onto structural homology models based on known crystal structure information. Homology models were built with MODPIPE (33) and graphics were created with UCSF Chimera software (34). Yellow spheres indicate mutated residues. (A) Two somatic mutations in the glycosylation enzyme GALNT5 occur in residues on different sides of the enzyme active site. Stick models indicate enzyme substrates. (B) Three somatic mutations in the transglutaminase TGM3 located at nearby surface regions of the protein (two mutations are present at the same residue on the right-hand side).

FIG. 2. PI3K pathway mutations in breast and colorectal cancers. The identities and relationships of genes that function in PI3K signaling are indicated. Circled genes have somatic mutations in colorectal (red) and breast (blue) cancers. The number of tumors with somatic mutations in each mutated protein is indicated by the number adjacent to the circle. Asterisks indicate proteins with mutated isoforms that may play similar roles in the cell. These include insulin receptor substrates IRS2 and IRS4; phosphatidylinositol 3-kinase regulatory subunits PIK3R1, PIK3R4, and PIK3R5; and nuclear factor kappa-B regulators NFKB1, NFKBIA, and NFKBIE.

FIG. 3. Cancer genome landscapes. Non-silent somatic mutations are plotted in two-dimensional space representing chromosomal positions of RefSeq genes. The telomere of the short arm of chromosome 1 is represented in the rear left corner of the green plane and ascending chromosomal positions continue in the direction of the arrow. Chromosomal positions that follow the front edge of the plane are continued at the back edge of the plane of the adjacent row and chromosomes are appended end to end. Peaks indicate the 60 highest-ranking CAN-genes for each tumor type, with peak heights reflecting CaMP scores (7). The dots represent genes that were somatically mutated in the individual colorectal (M×38) or breast tumor (B3C) displayed. The dots corresponding to mutated genes that coincided with hills or mountains are black with white rims; the remaining dots are white with red rims. The mountain on the right of both landscapes represents TP53 (chromosome 17), and the other mountain shared by both breast and colorectal cancers is PIK3CA (upper left, chromosome 3).

FIG. 4. (FIG. S1.) Schematic of the experimental and bioinformatic approaches used in the study

FIG. 5. Table 1. Summary of somatic mutations

FIG. 6. Table S1. Primers used for PCR amplification and sequencing

FIG. 7. Table S2. Distribution of somatic mutations in individual tumors

FIG. 8. Table S3. Somatic mutations discovered in RefSeq genes

FIG. 9 (Table S4A) Colorectal CAN-genes

FIG. 10 (Table S4B) Breast CAN-genes

FIG. 11. Table S5. Summary of mutation prevalence study

FIG. 12. Table S6A. Gene groups and pathways preferentially mutated in colorectal cancers

FIG. 13. Table S6B. Gene groups and pathways preferentially mutated in breast cancers

### DETAILED DESCRIPTION OF THE INVENTION

The inventors have developed methods for characterizing breast and colorectal cancers on the basis of gene signatures. These signatures comprise one or more genes which are mutated in a particular cancer. The signatures can be used as a means of diagnosis, prognosis, identification of metastasis, stratification for drug studies, and for assigning an appropriate treatment.

According to the present invention a mutation, typically a somatic mutation, can be determined by testing either a gene, its mRNA (or derived cDNA), or its encoded protein. Any method known in the art for determining a somatic mutation can be used. The method may involve sequence determination of all or part of a gene, cDNA, or protein. The method may involve mutation-specific reagents such as probes, primers, or antibodies. The method may be based on amplification, hybridization, antibody-antigen reactions, primer extension, etc. Any technique or method known in the art for determining a sequence-based feature may be used.

Samples for testing may be tissue samples from breast or colorectal tissue or body fluids or products that contain sloughed off cells or genes or mRNA or proteins. Such fluids or products include breast milk, stool, breast discharge, intestinal fluid. Preferably the same type of tissue or fluid is used for the test sample and the normal sample. The test sample is, however, suspected of possible neoplastic abnormality, while the normal sample is not suspect.

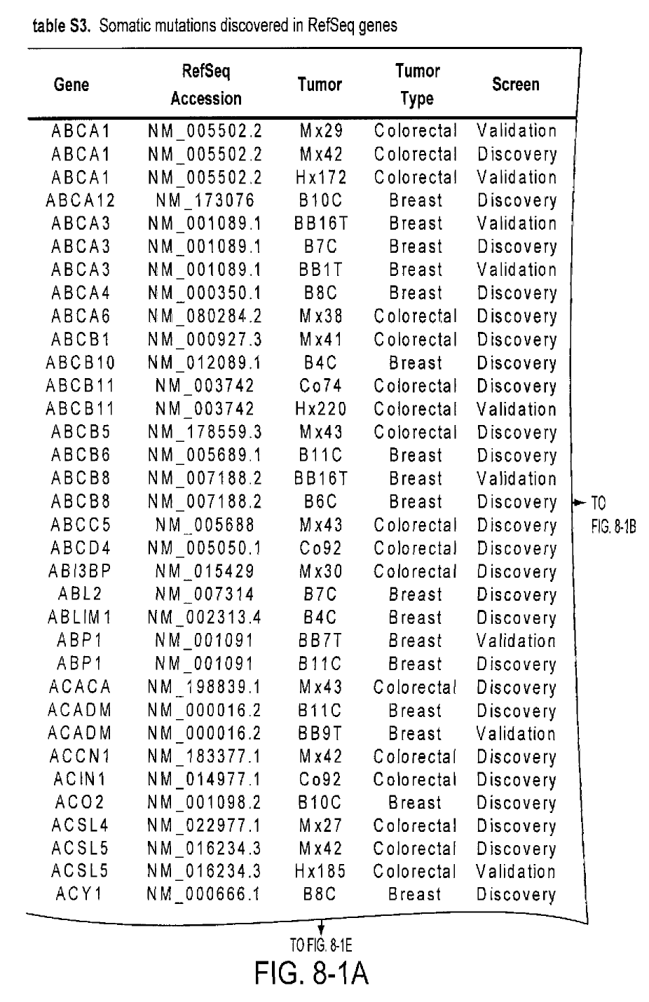

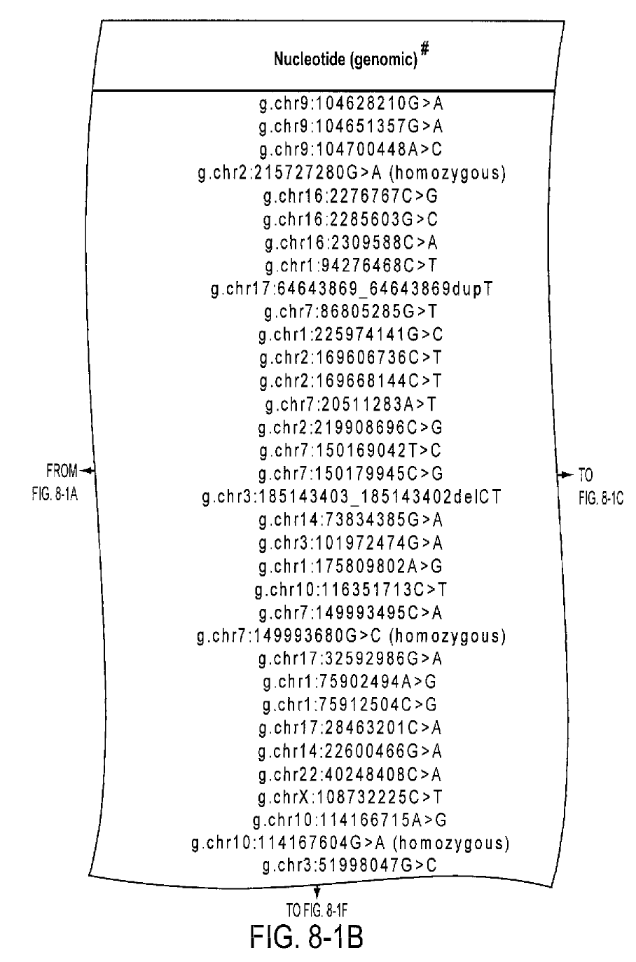

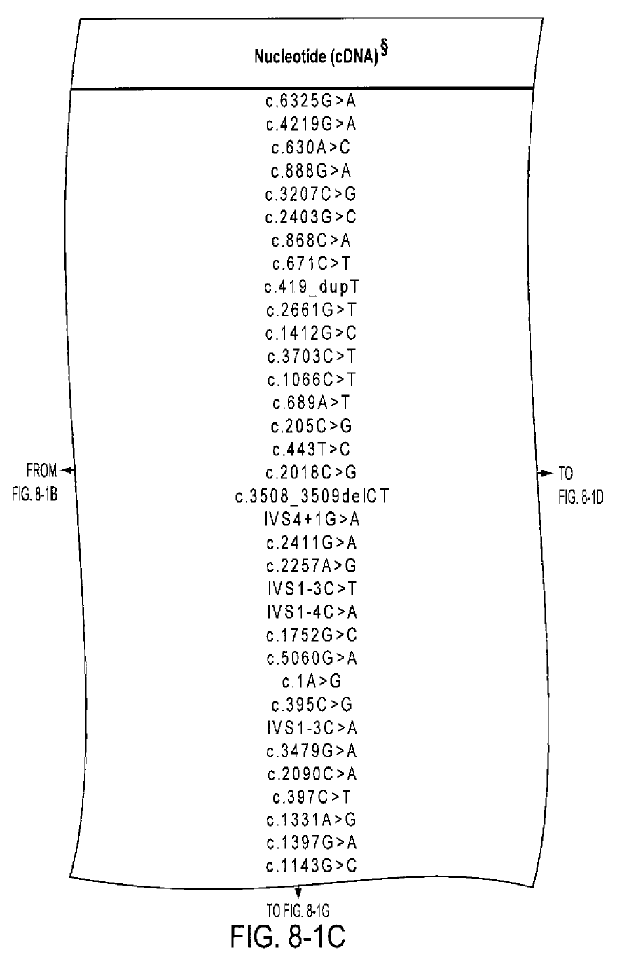

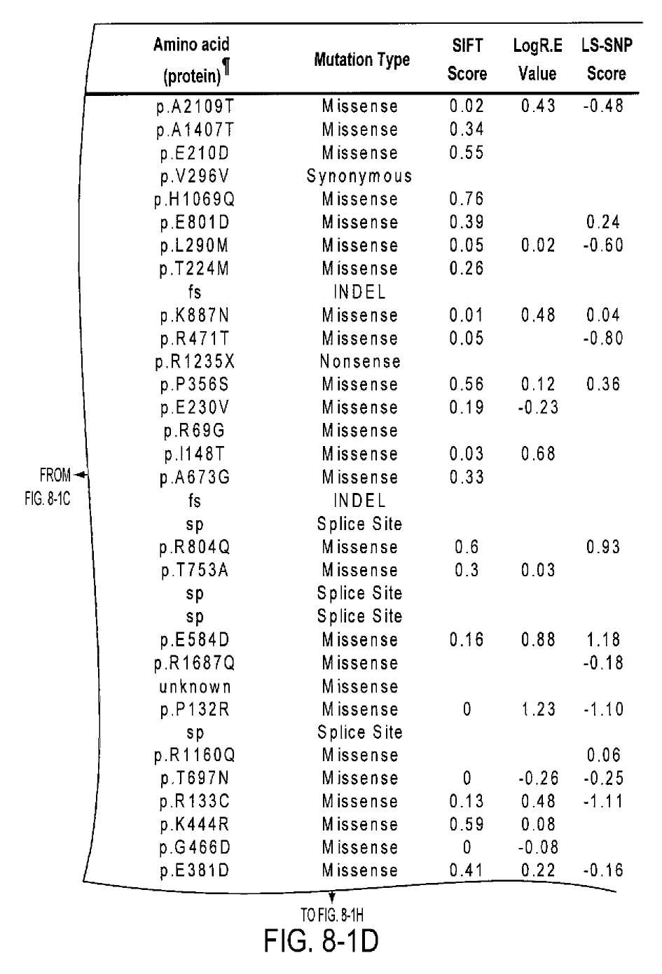

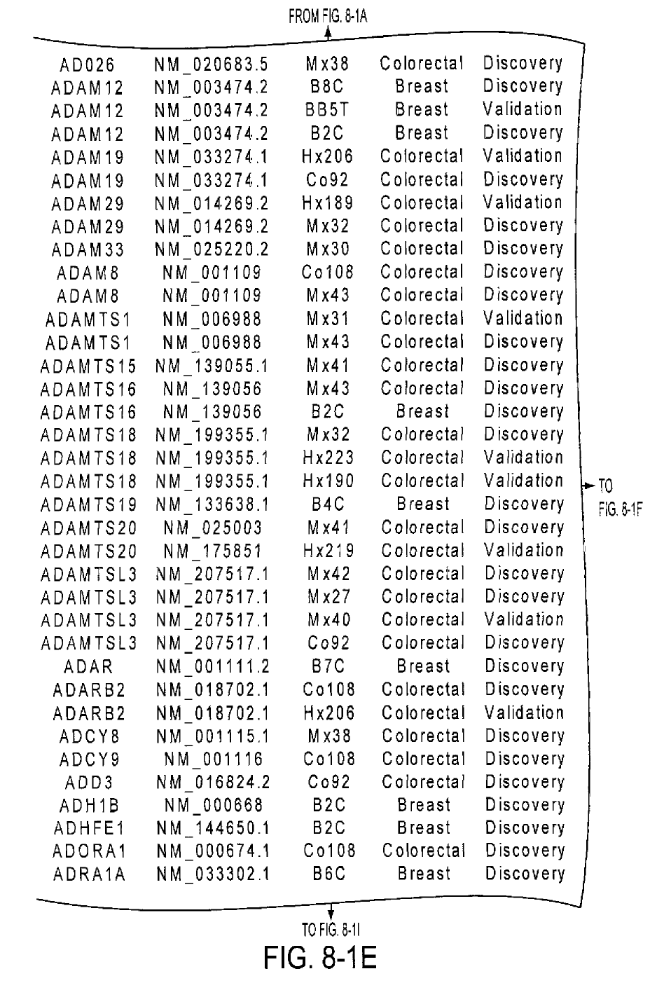

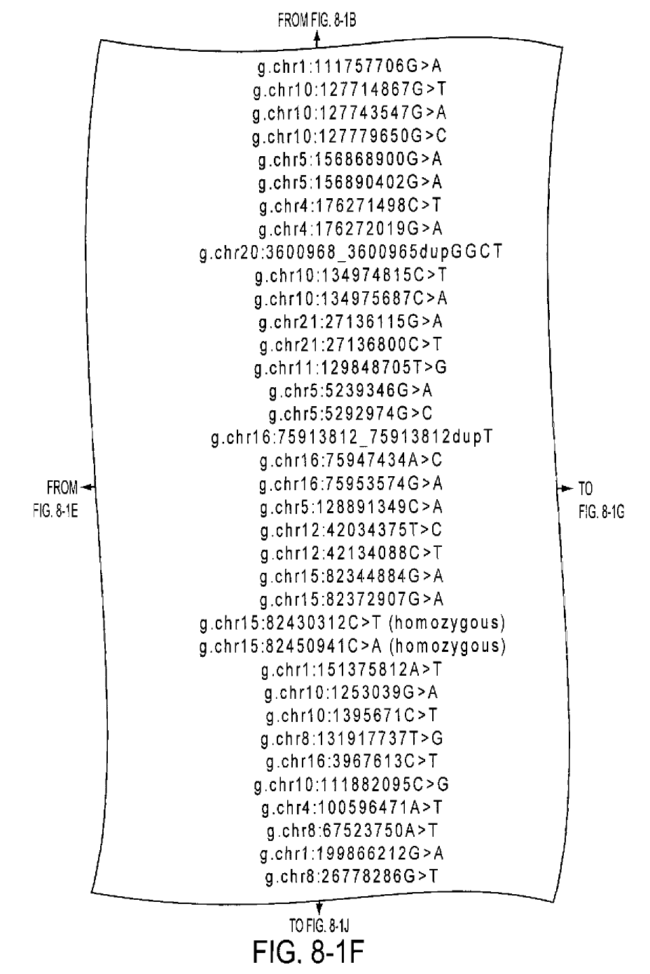

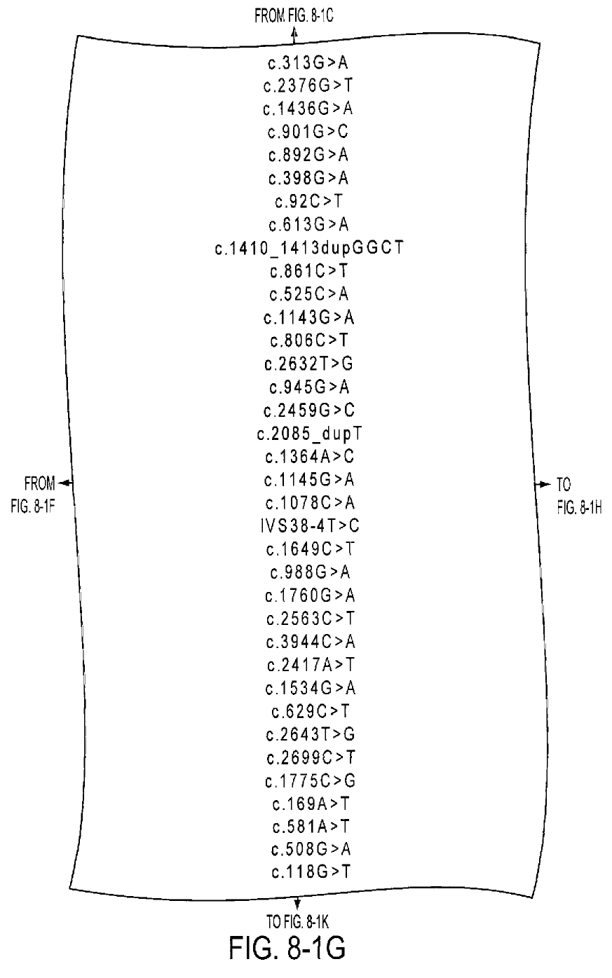

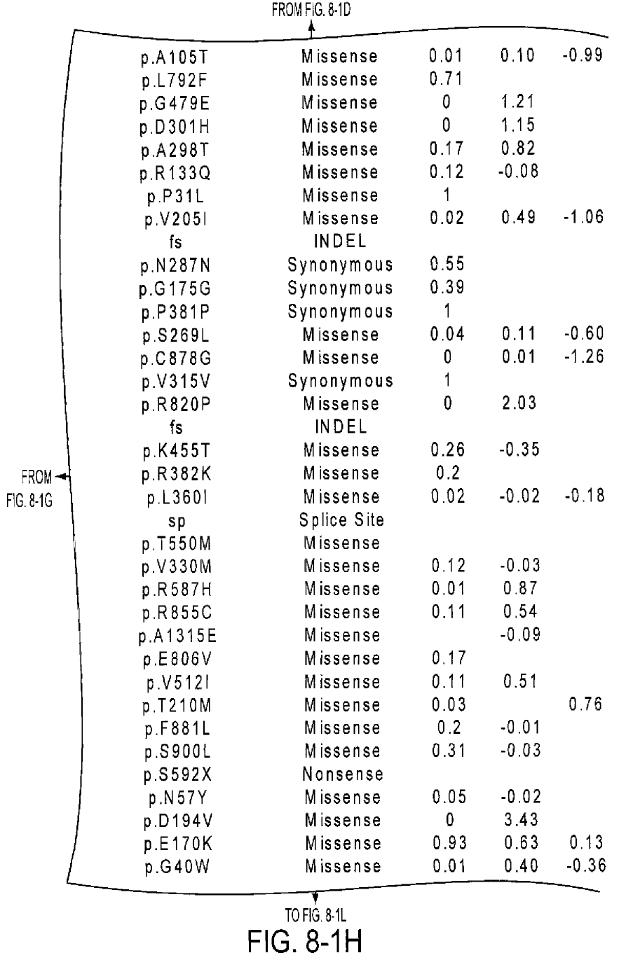

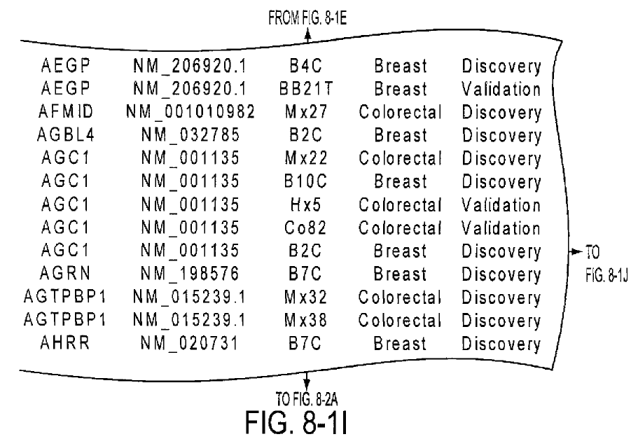

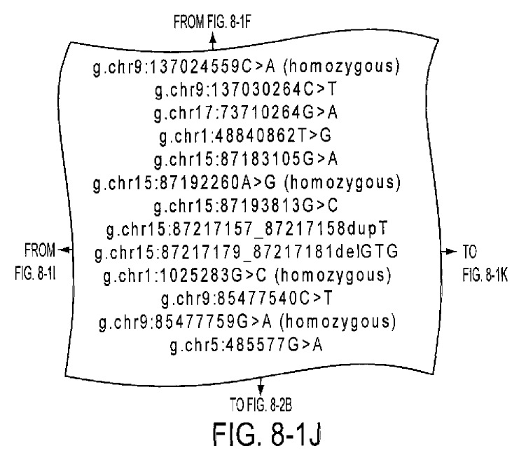

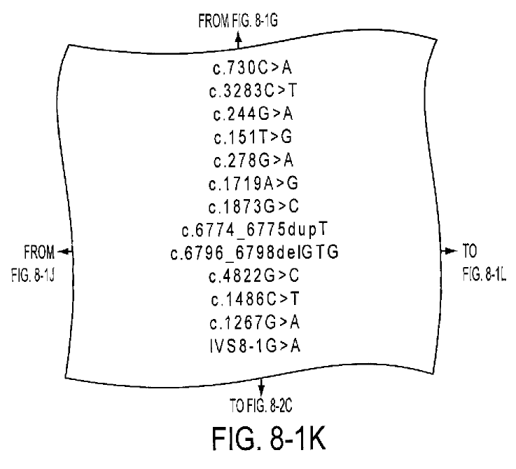

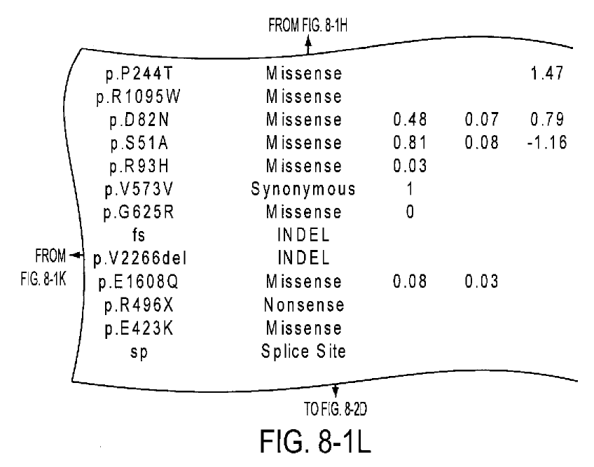

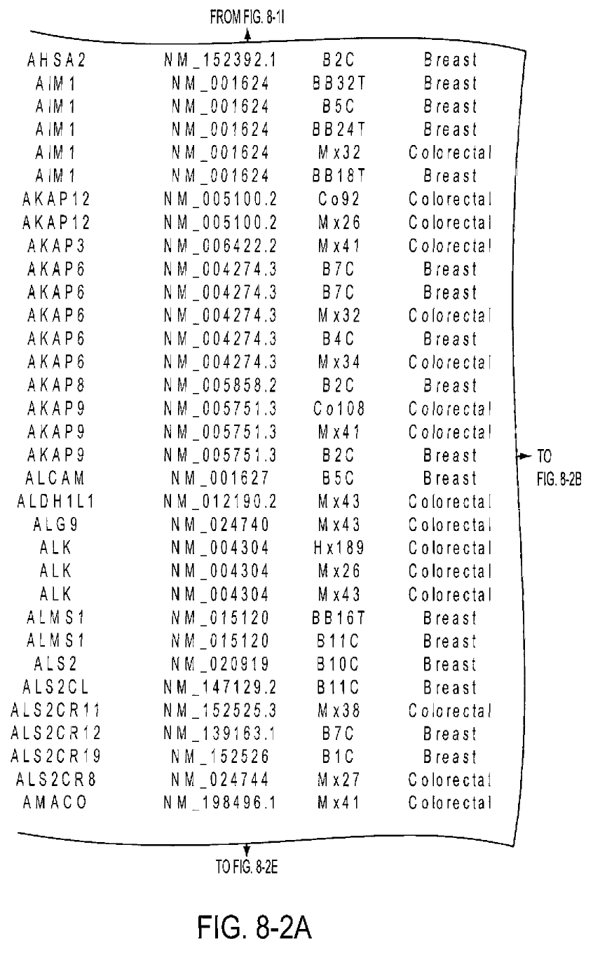

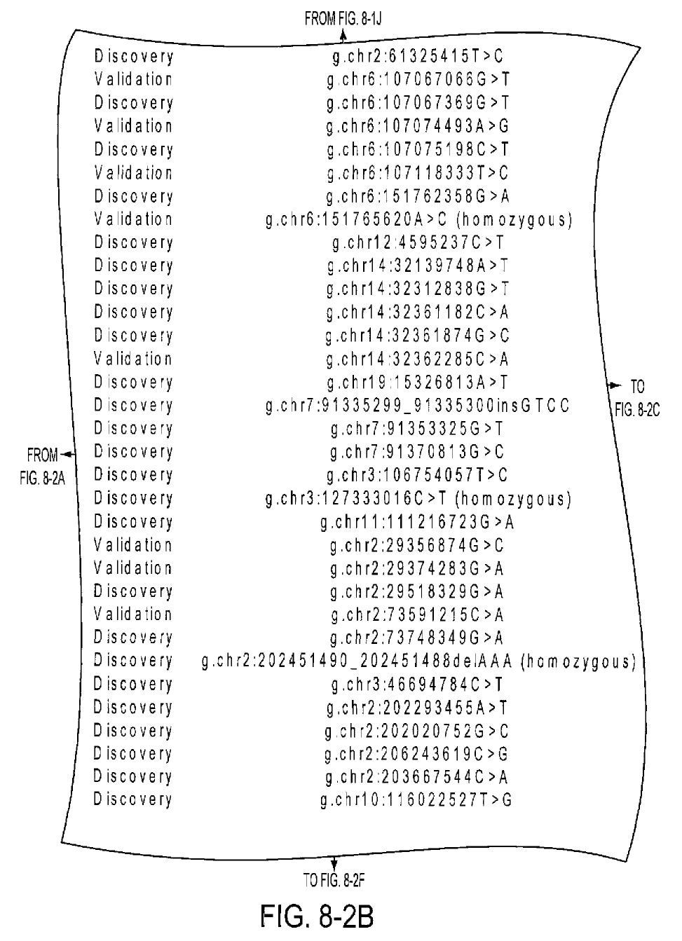

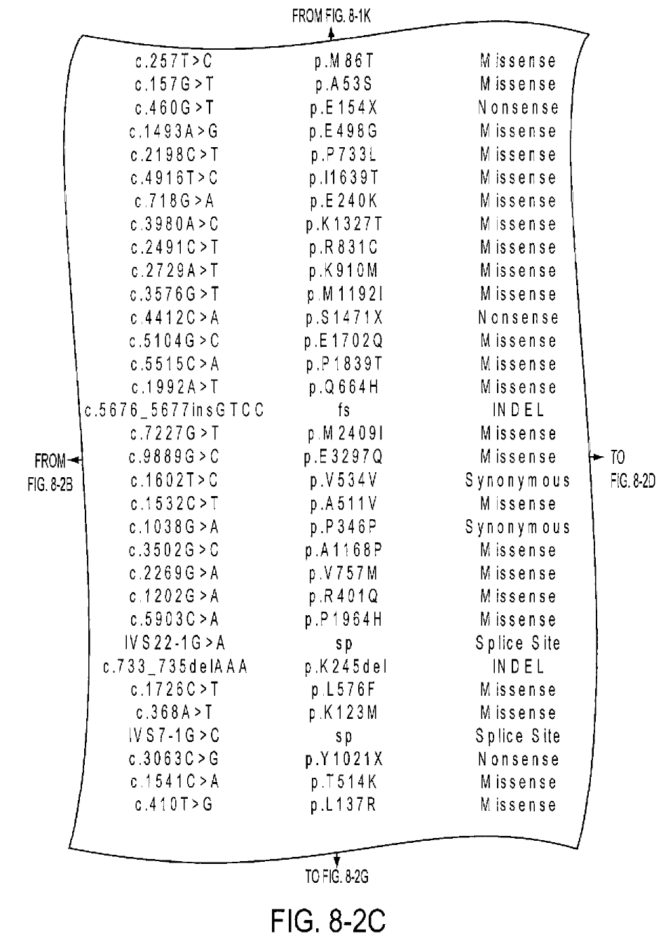

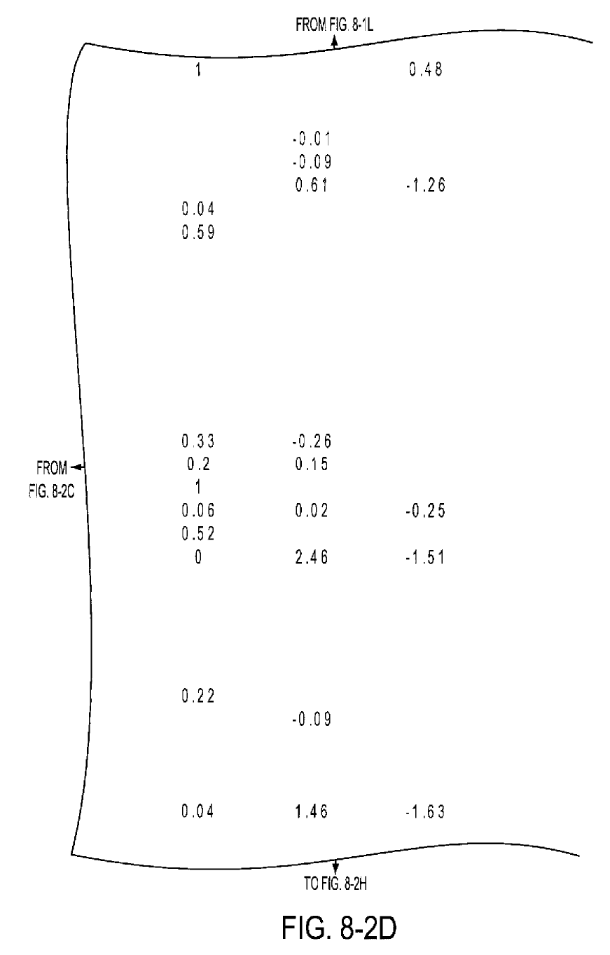

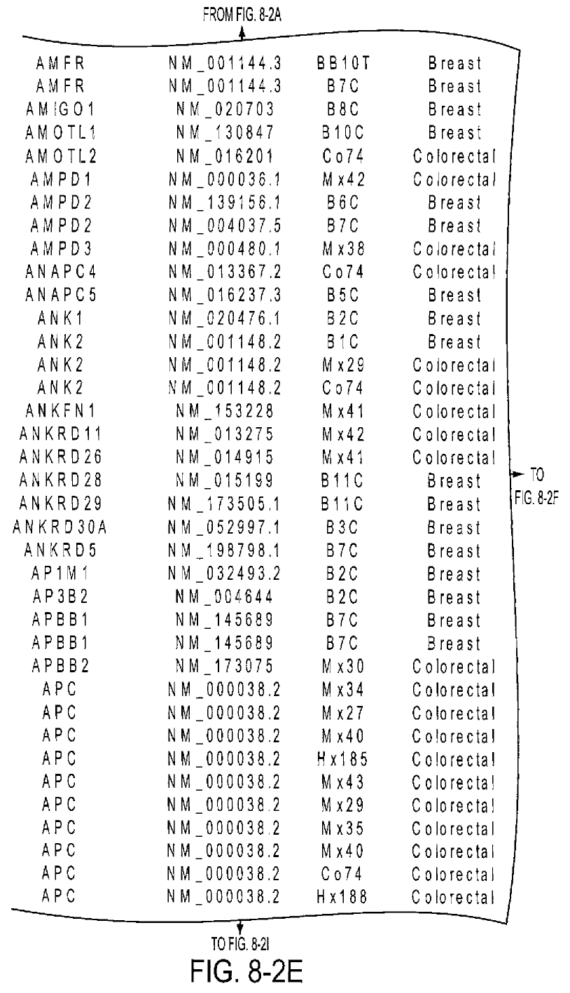

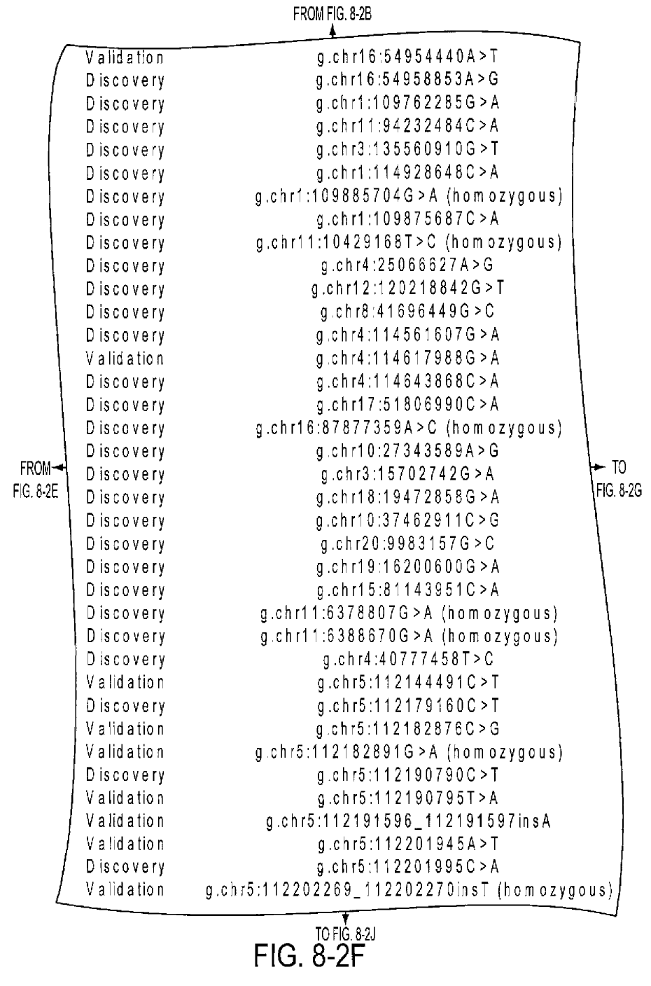

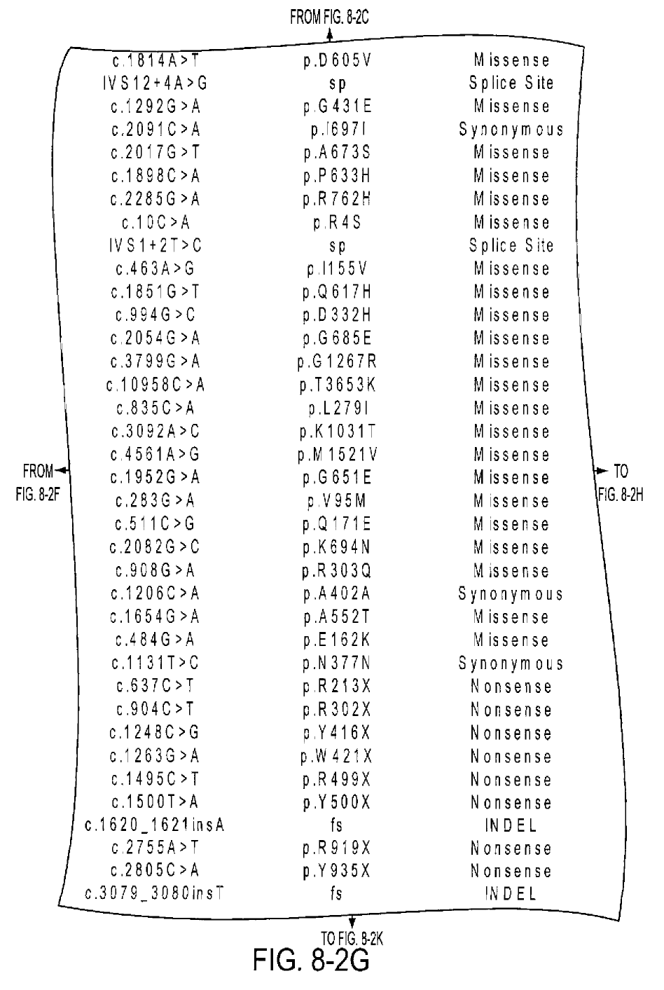

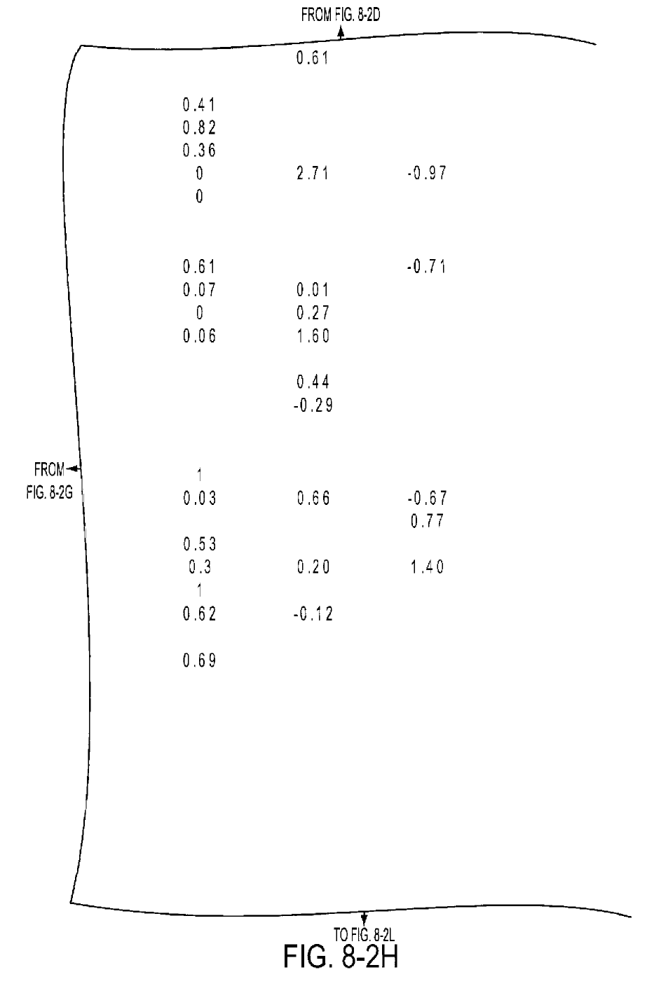

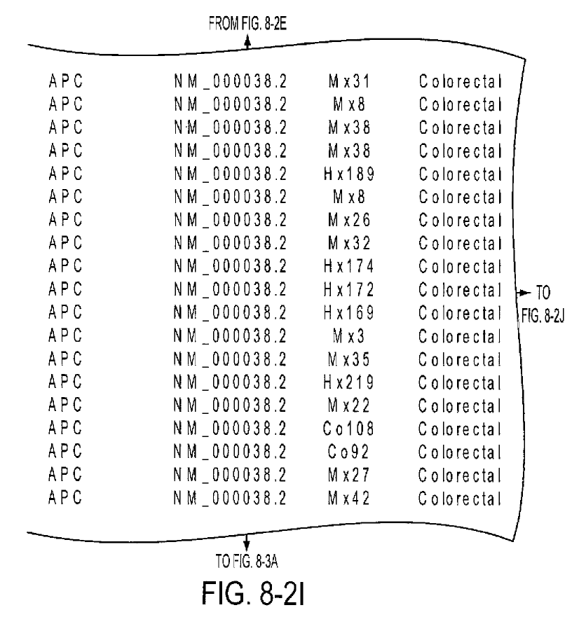

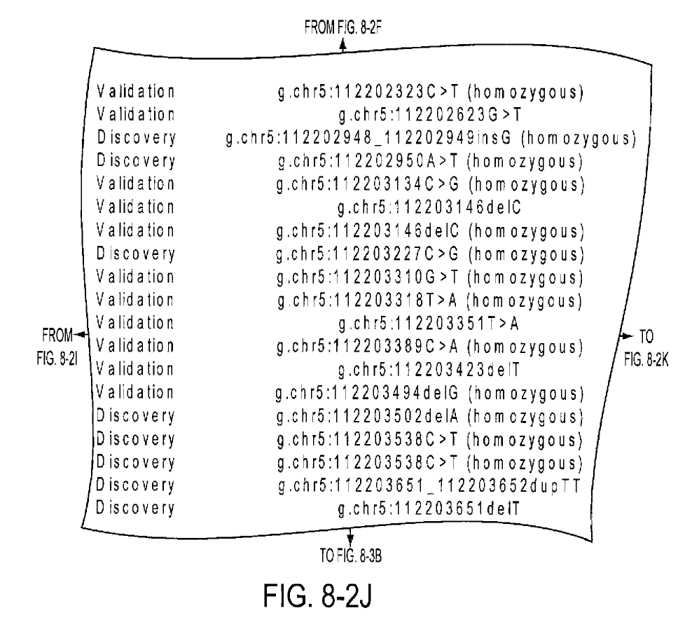

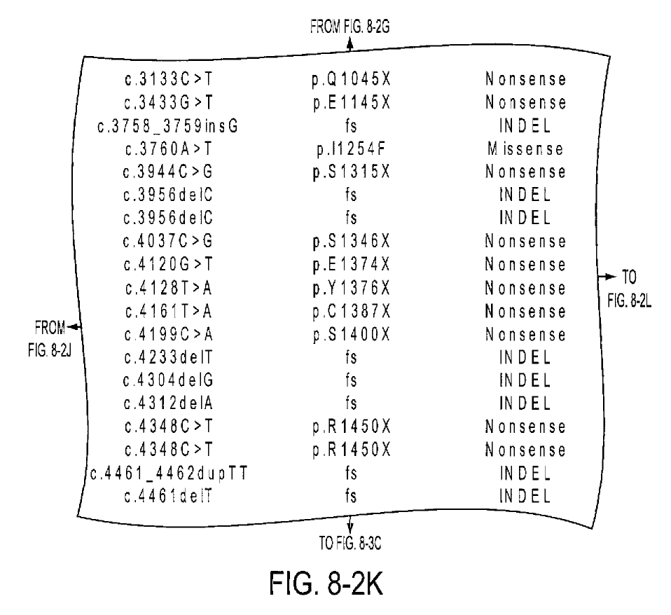

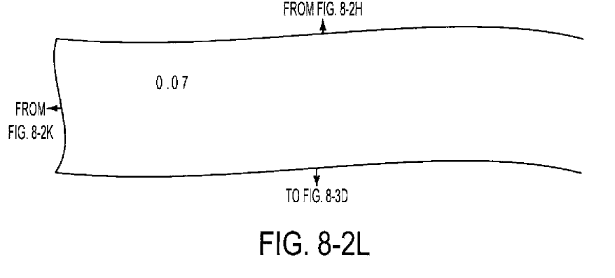

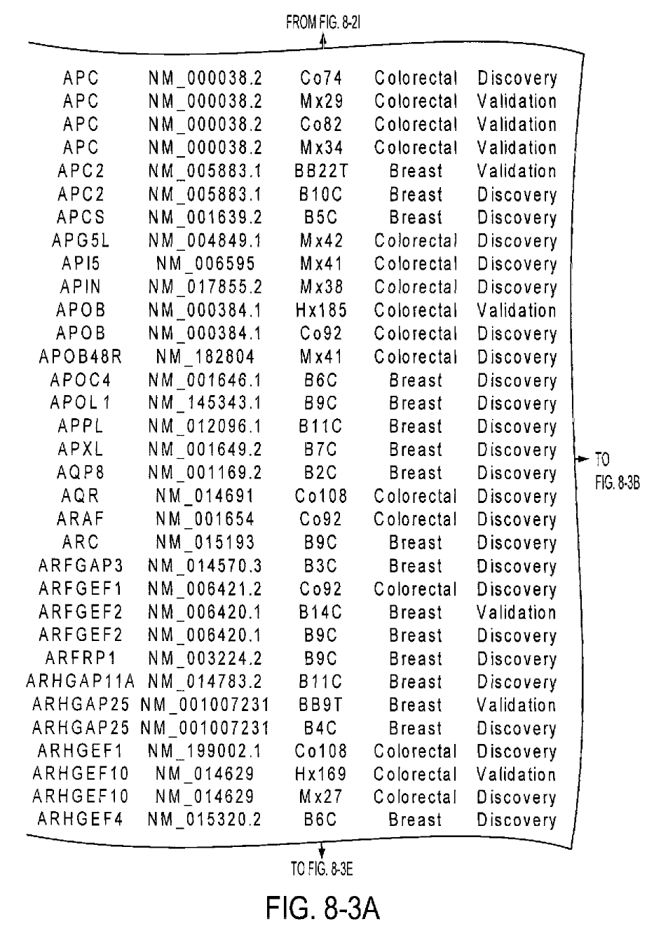

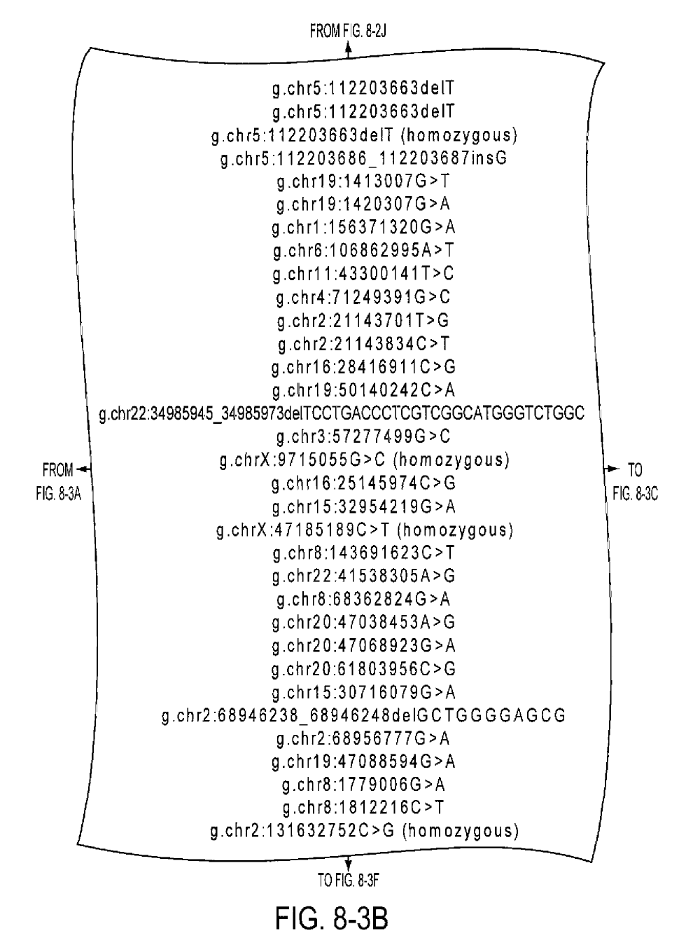

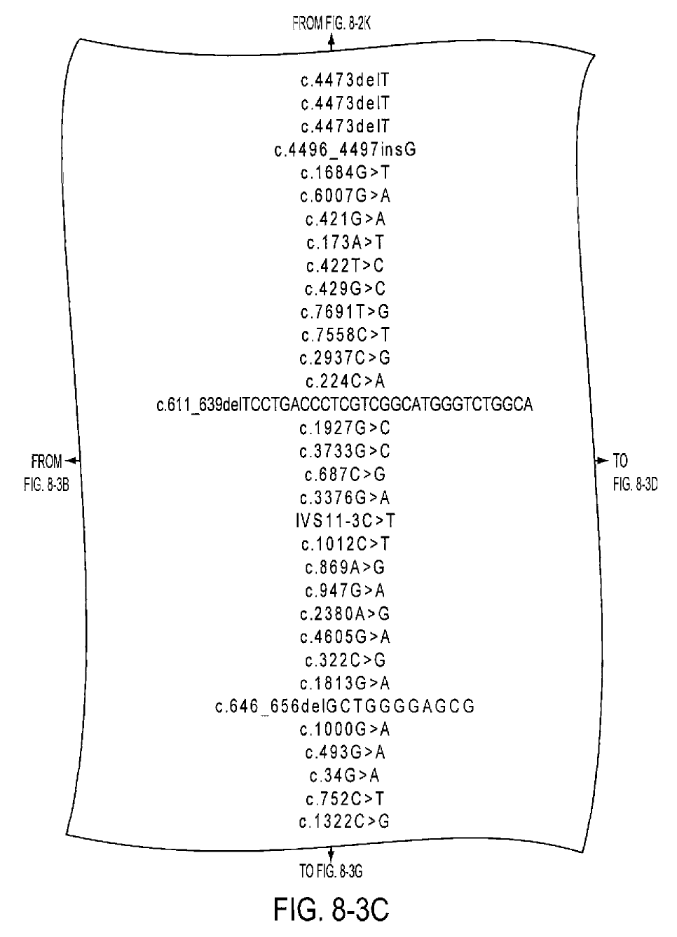

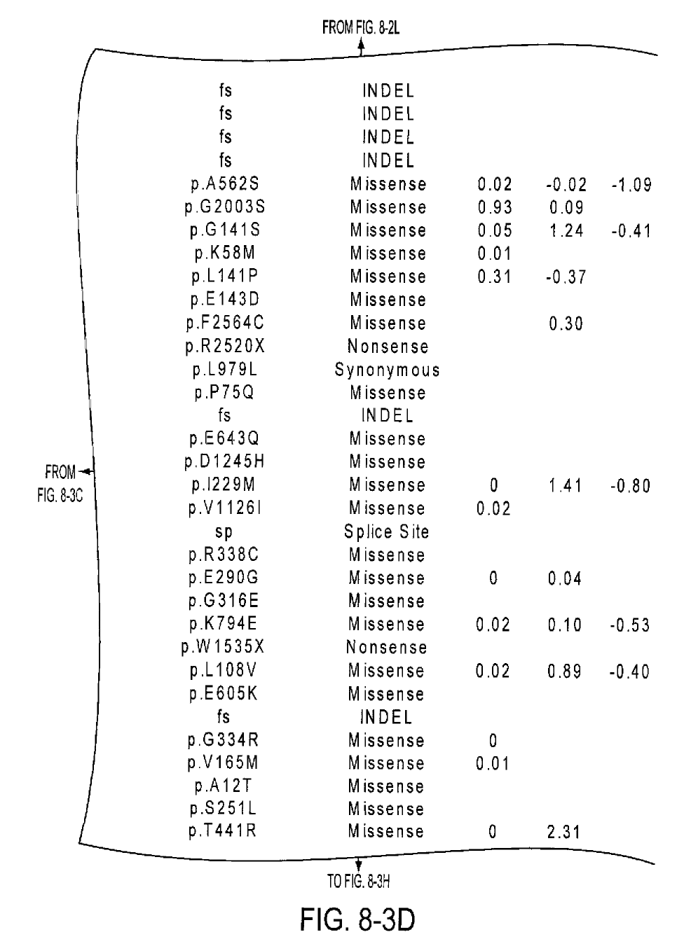

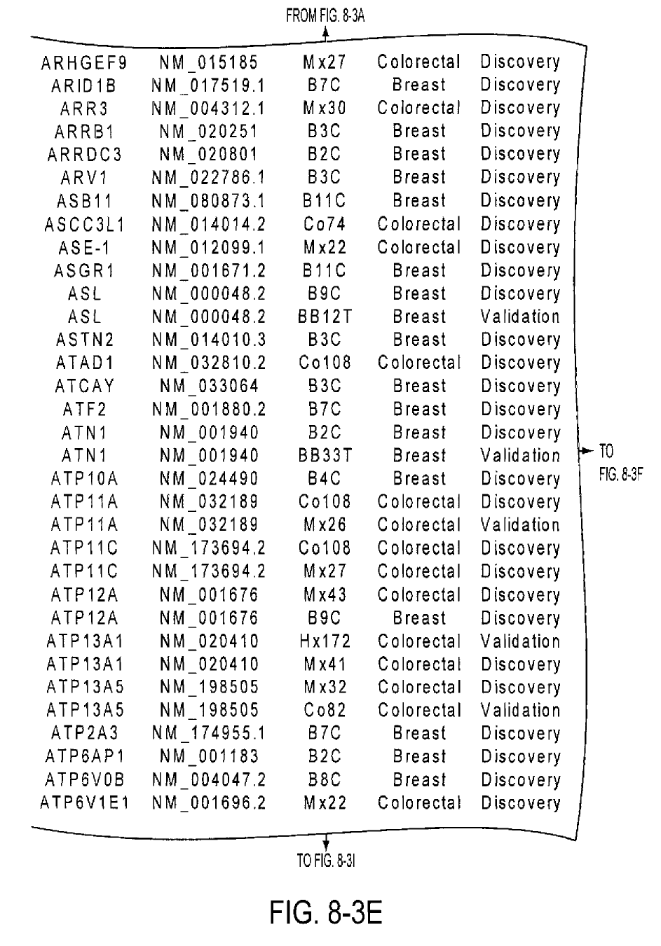

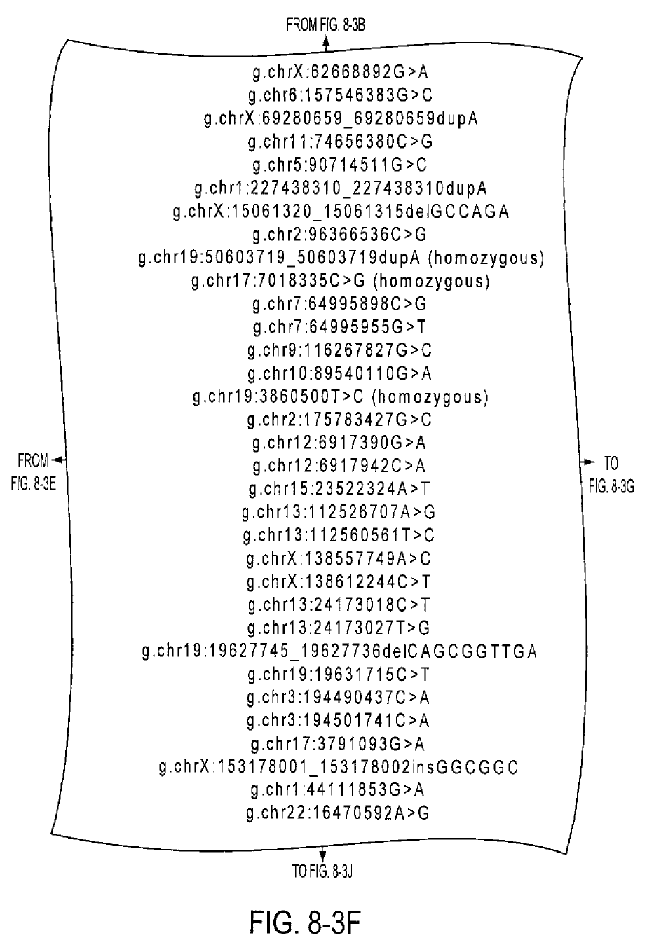

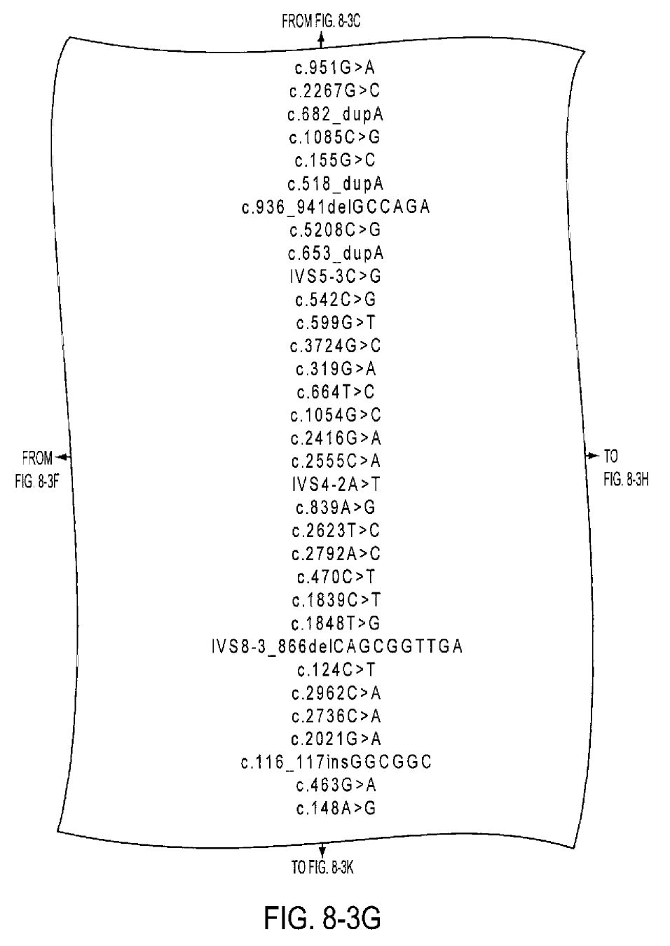

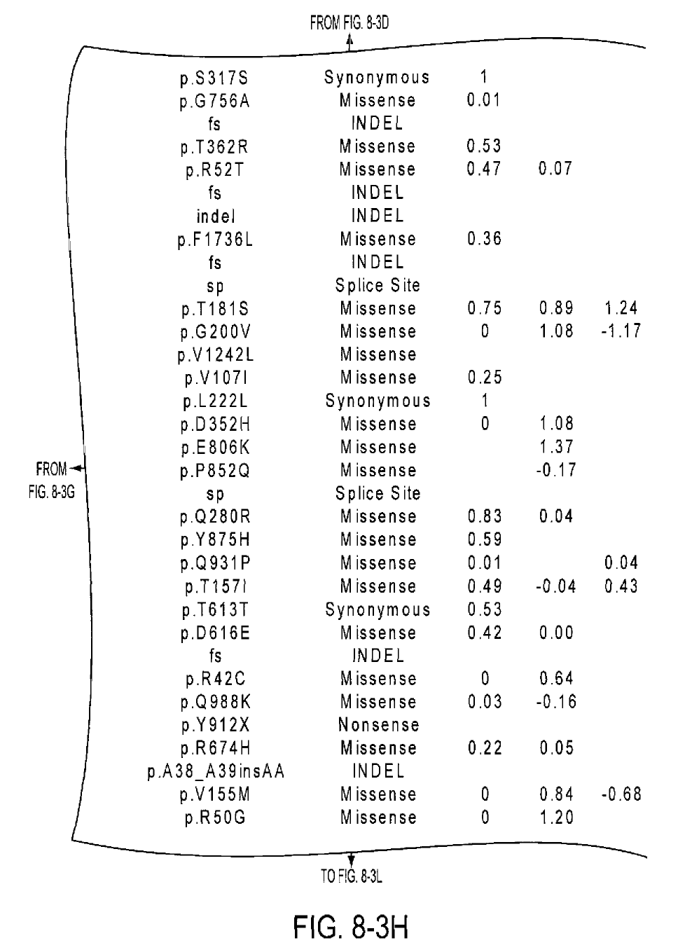

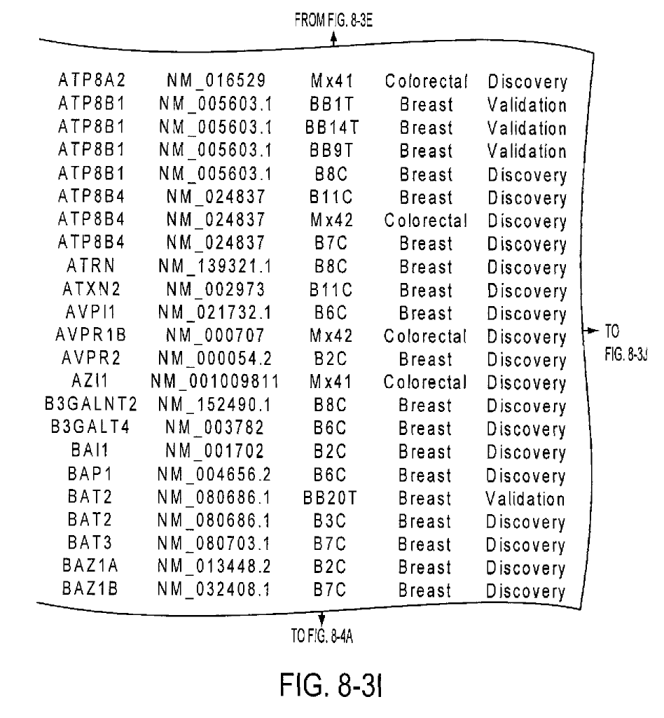

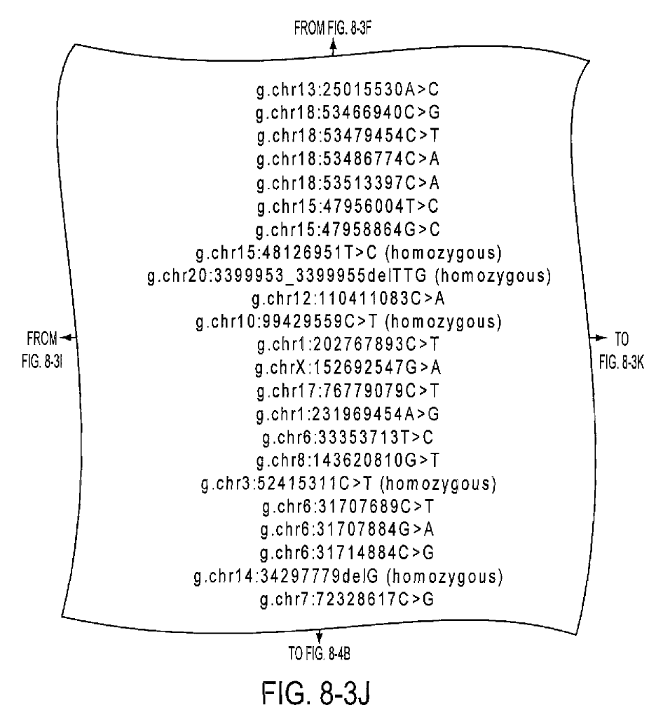

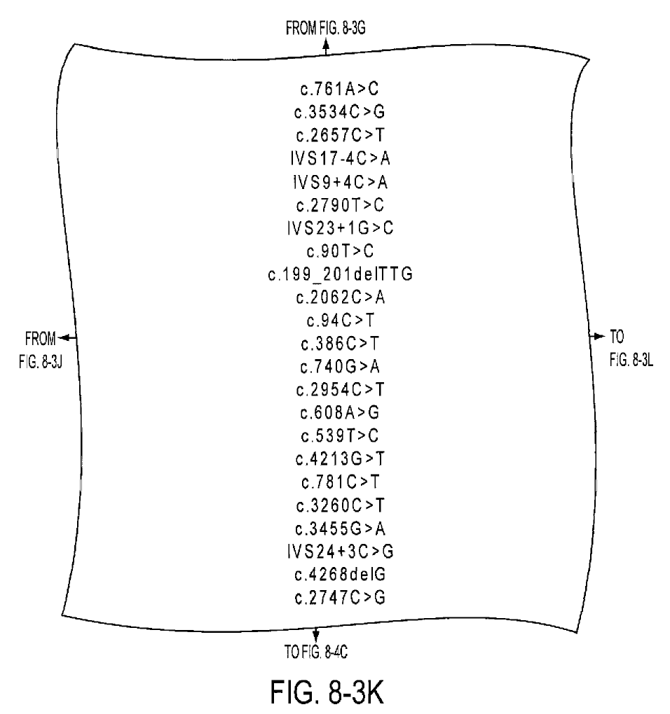

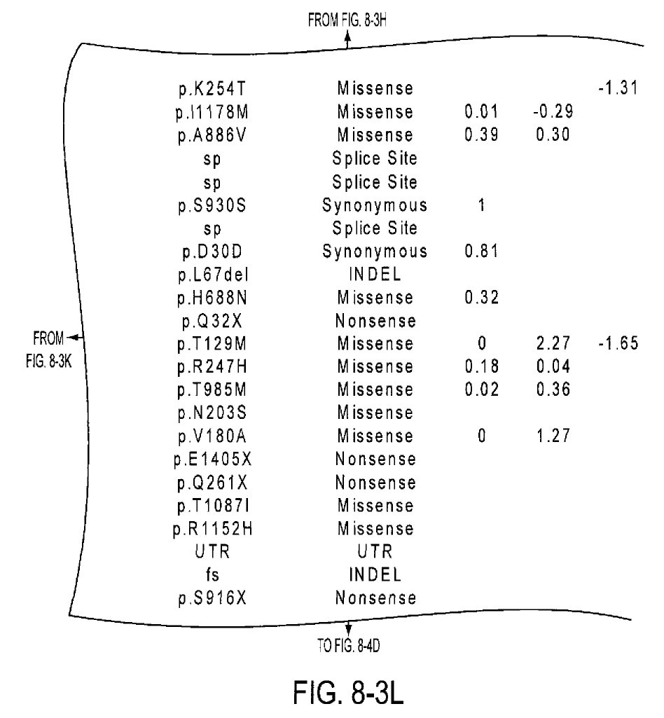

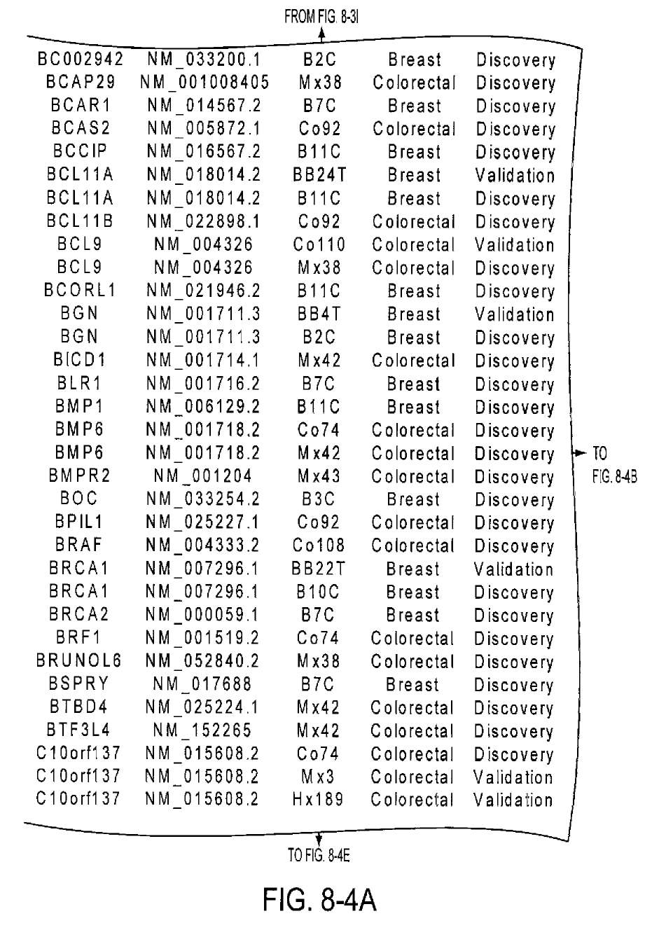

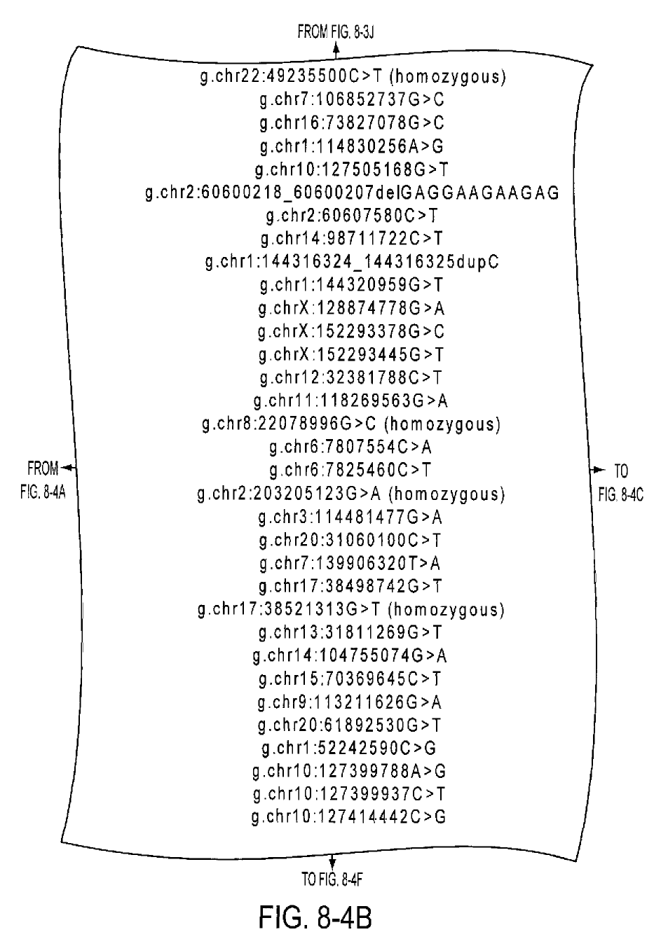

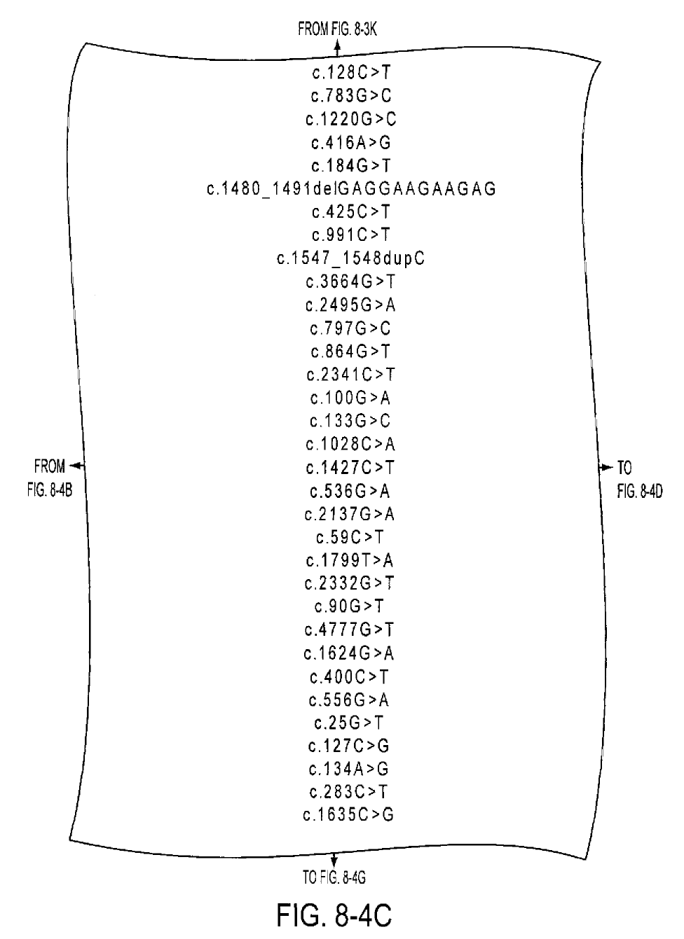

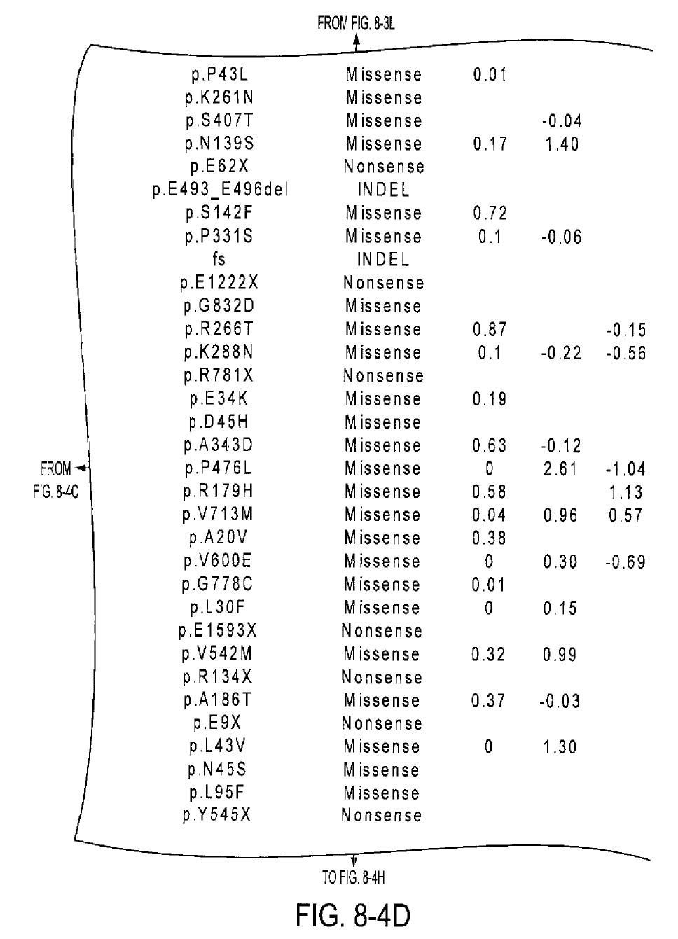

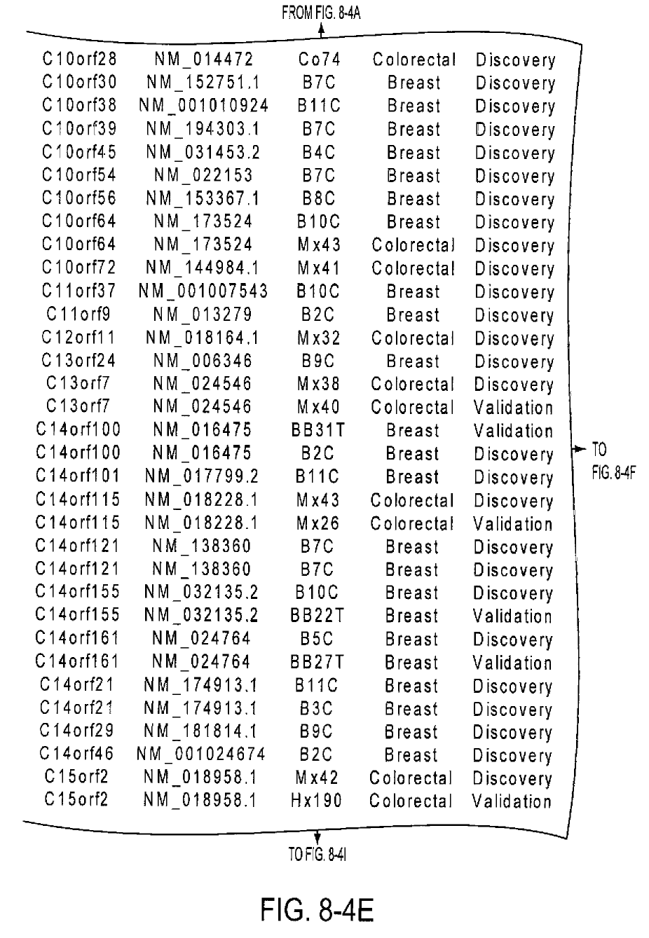

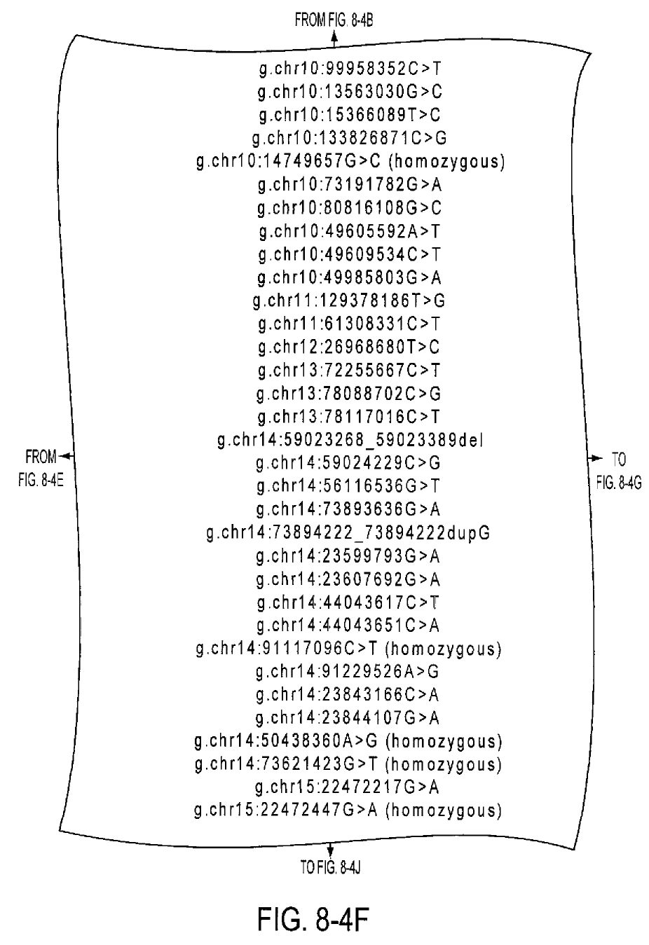

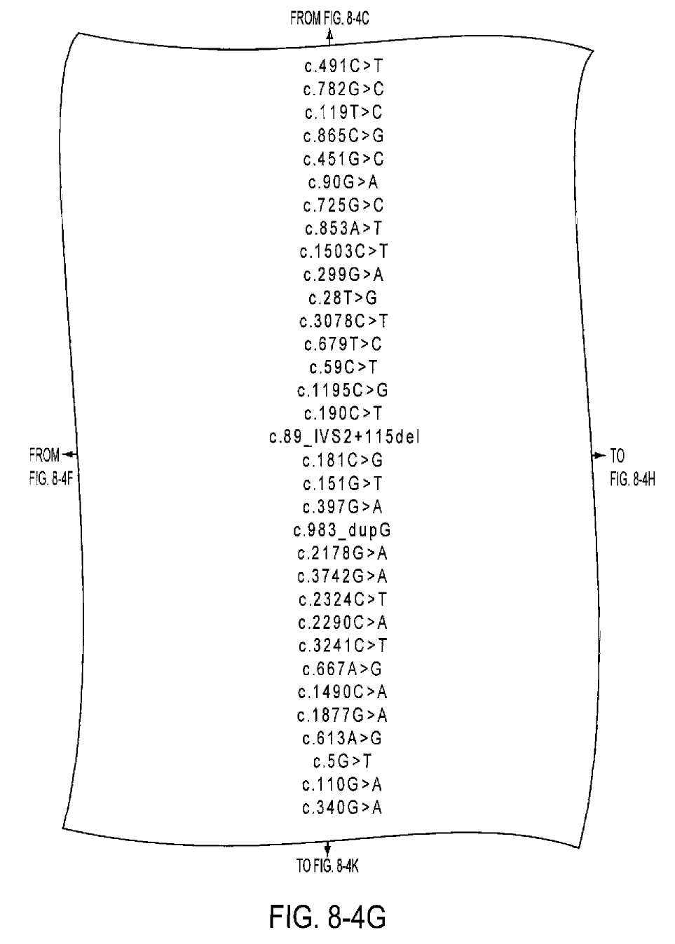

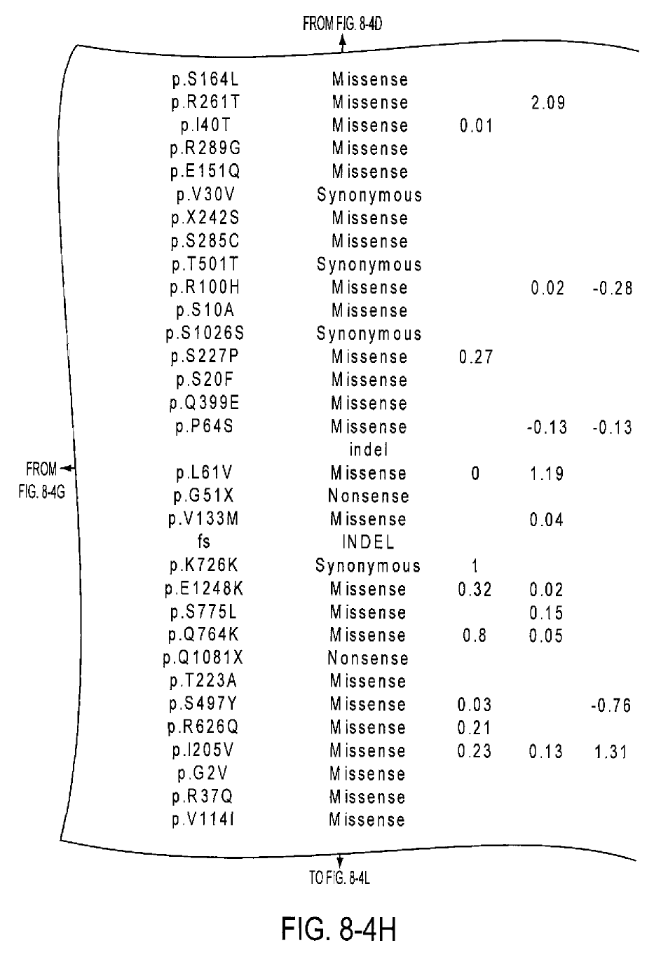

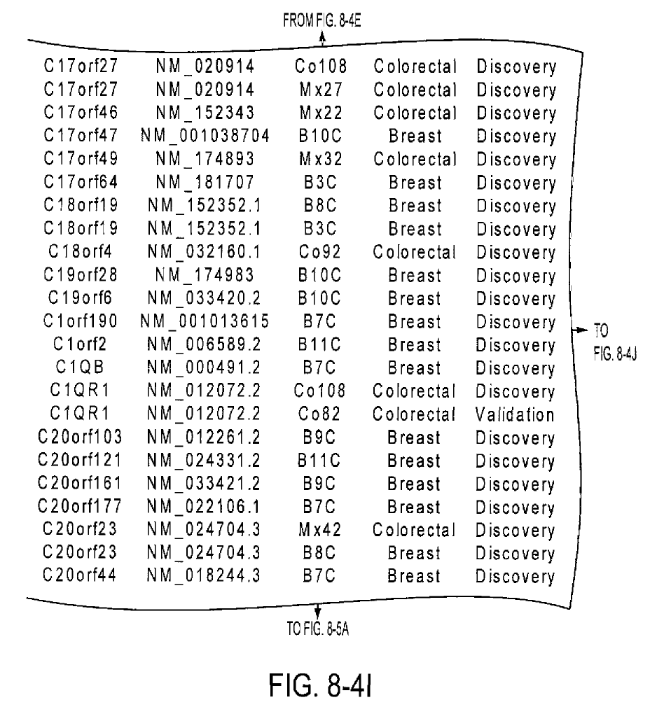

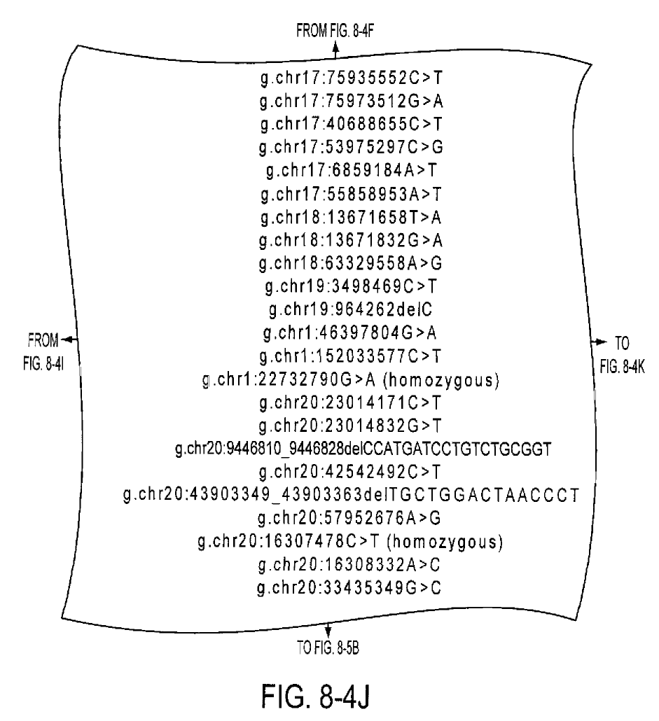

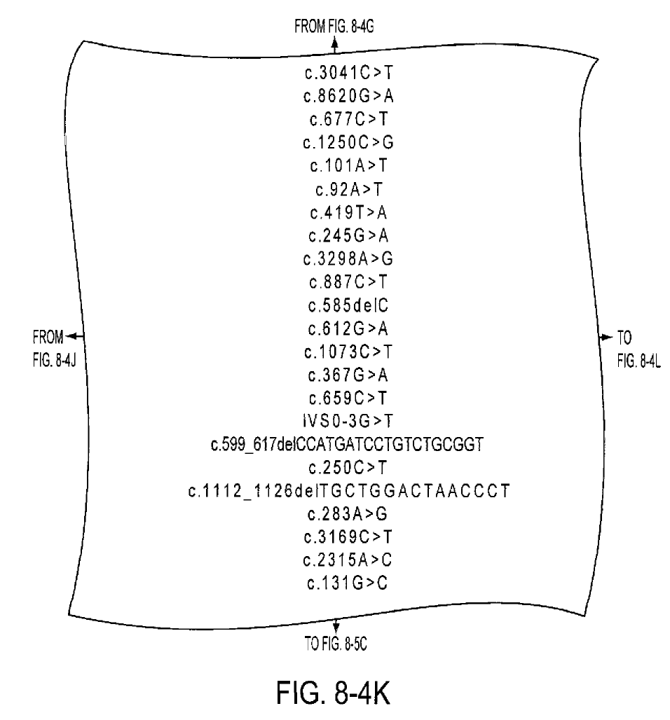

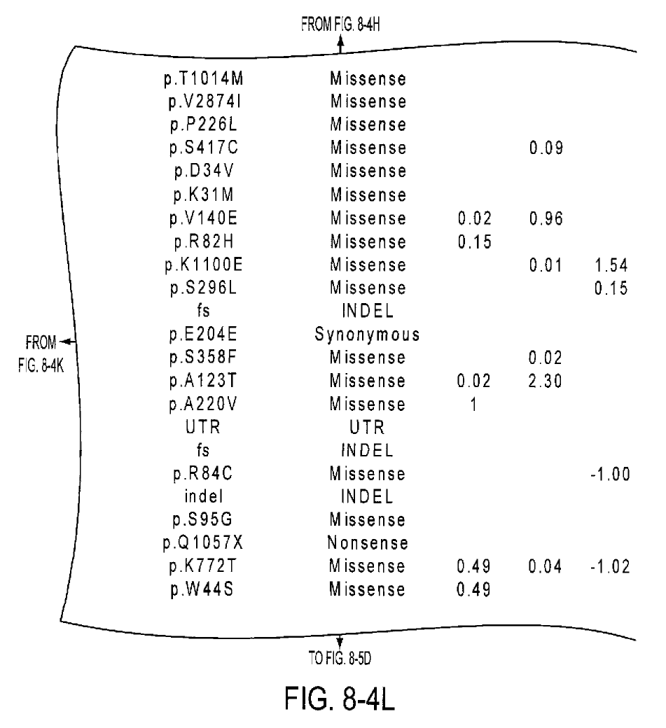

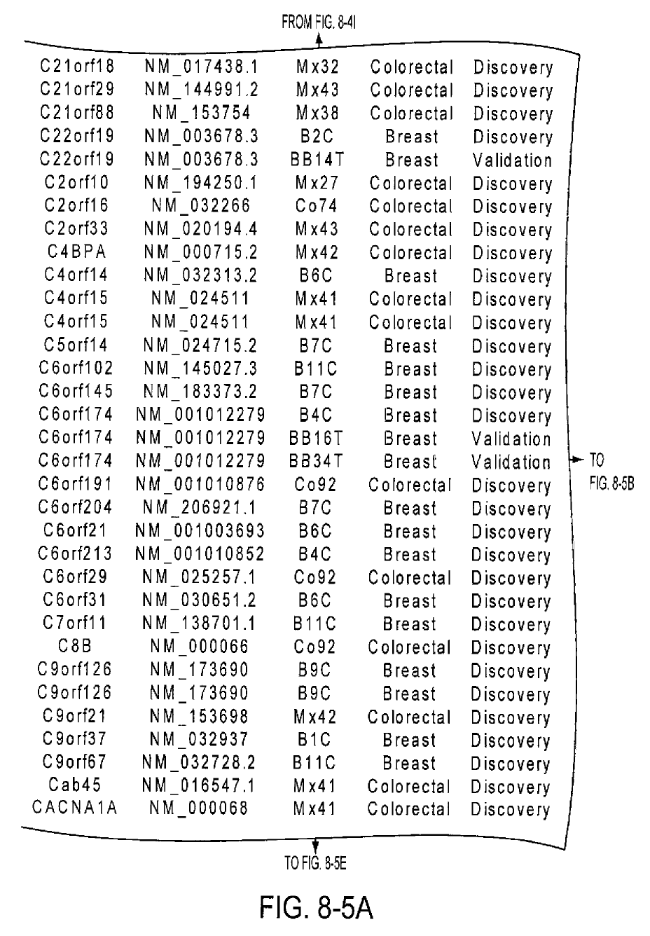

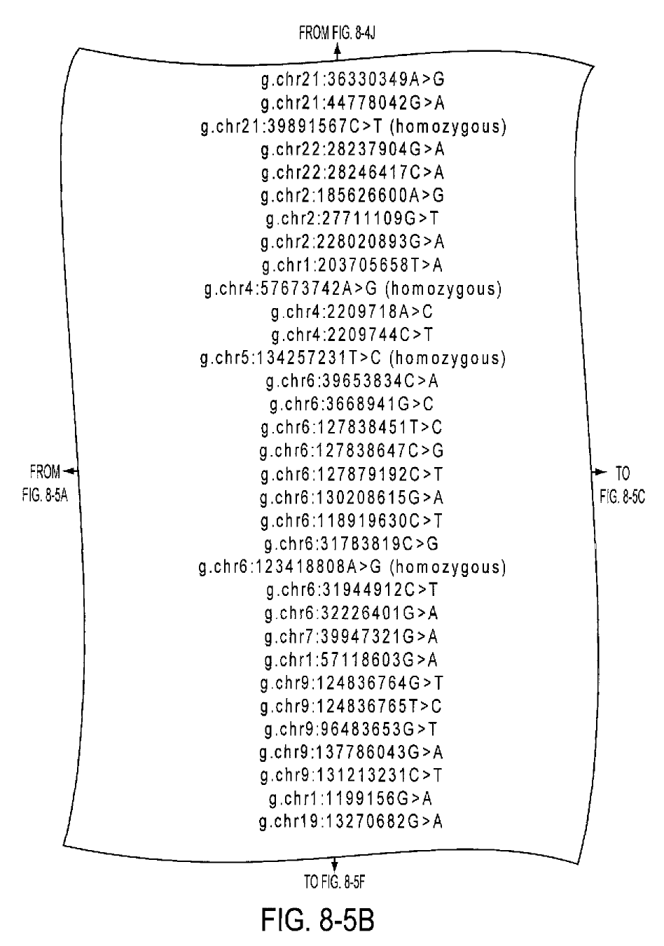

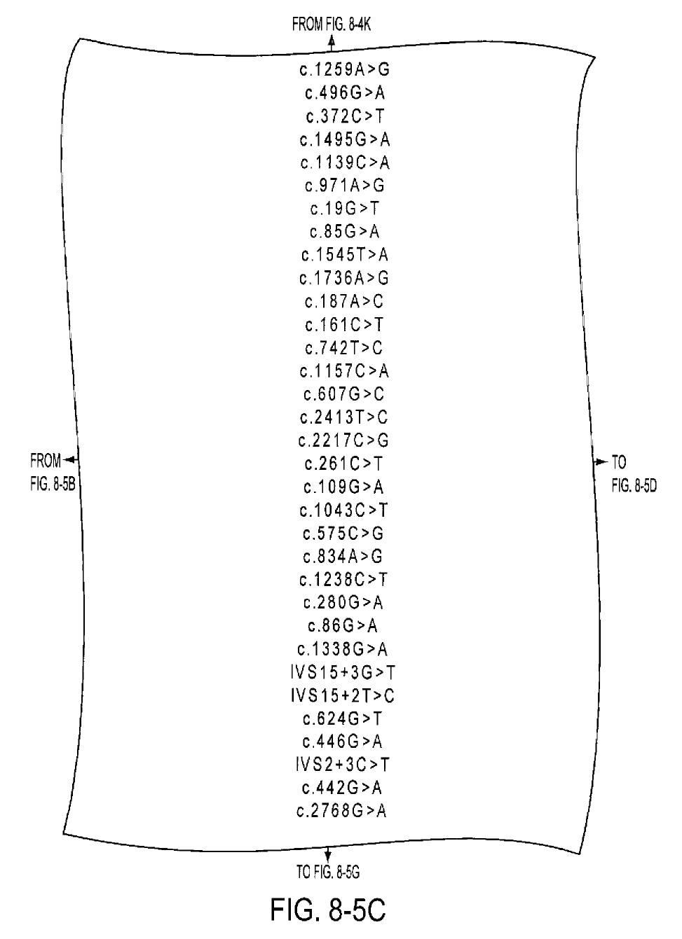

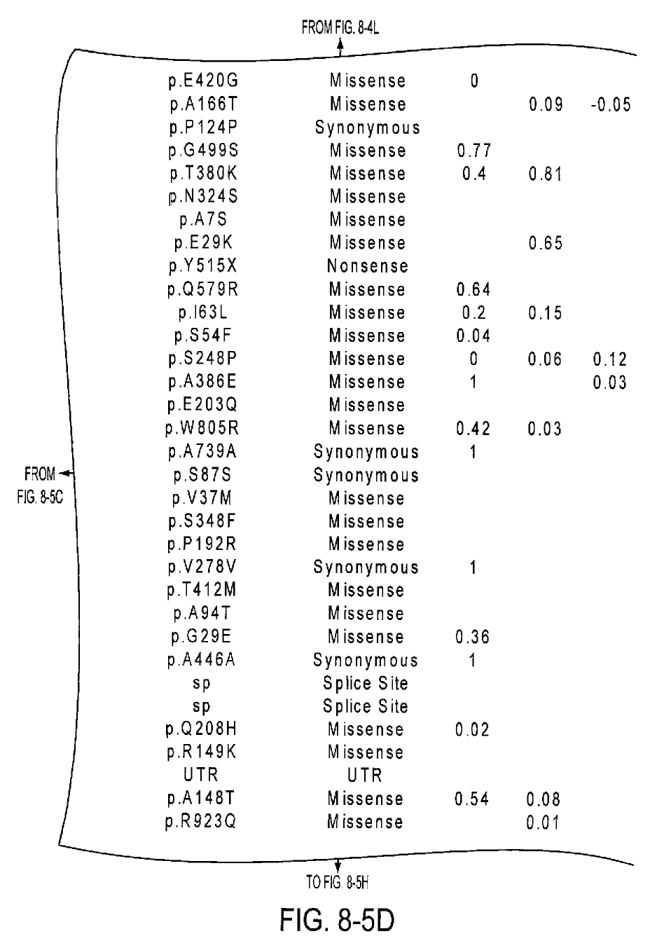

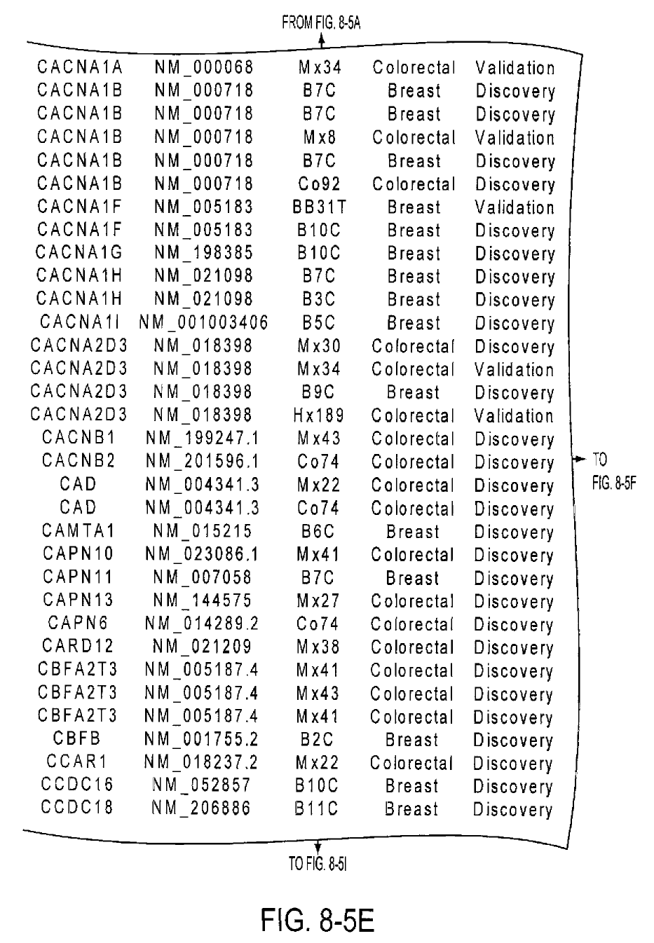

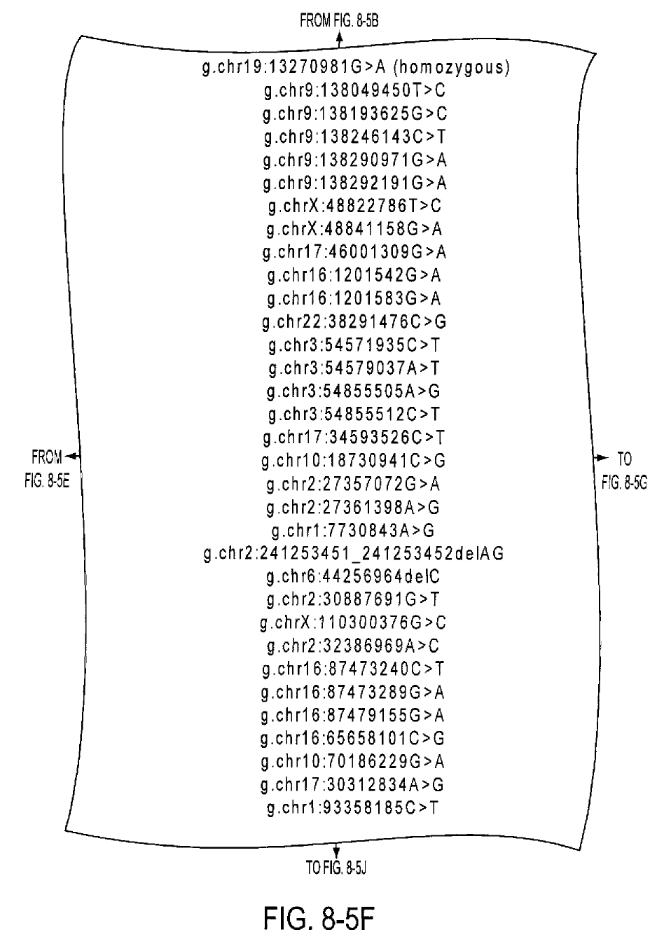

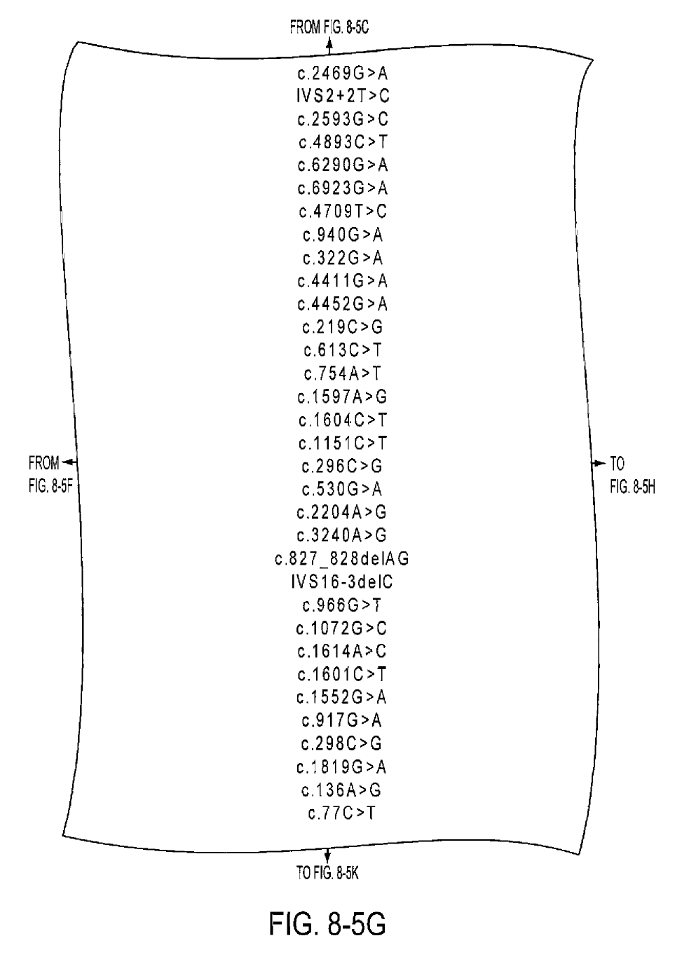

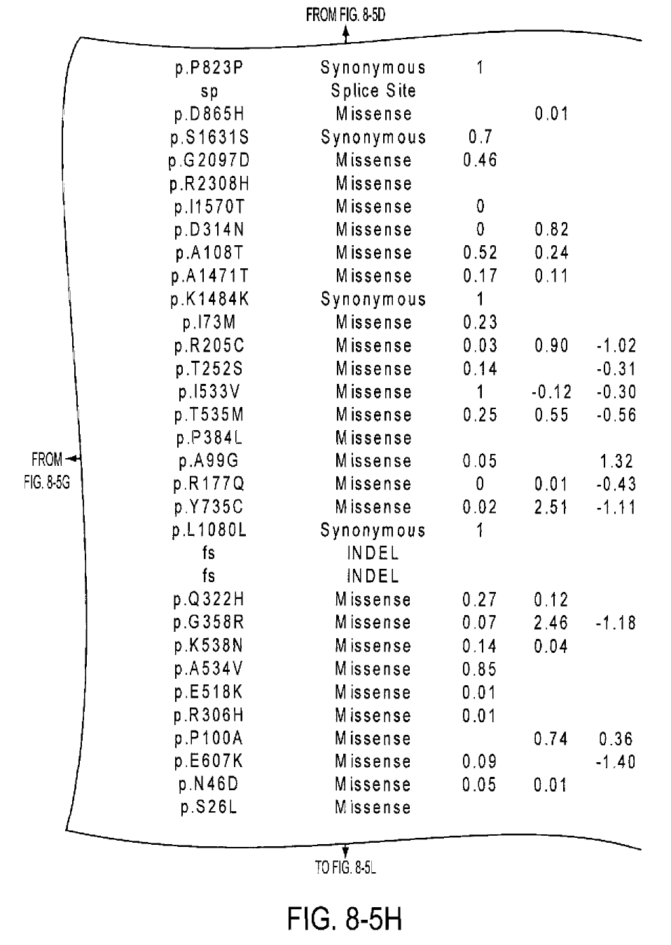

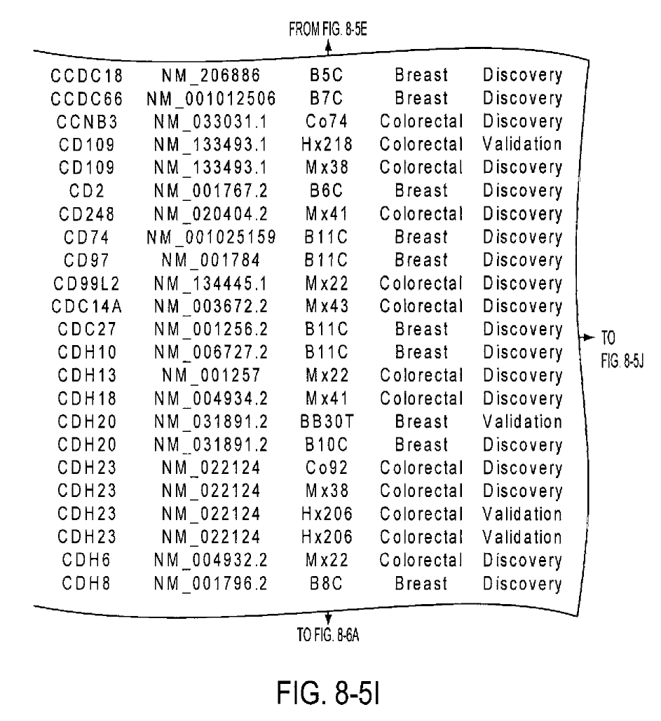

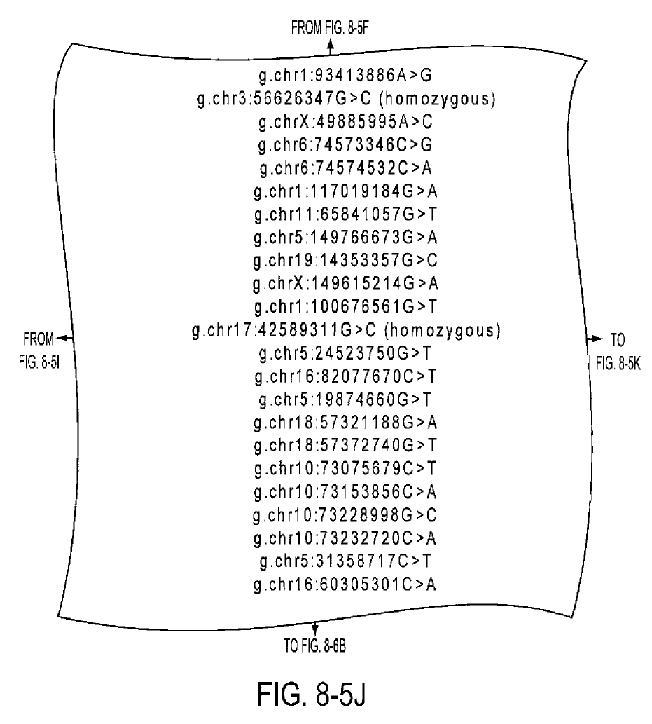

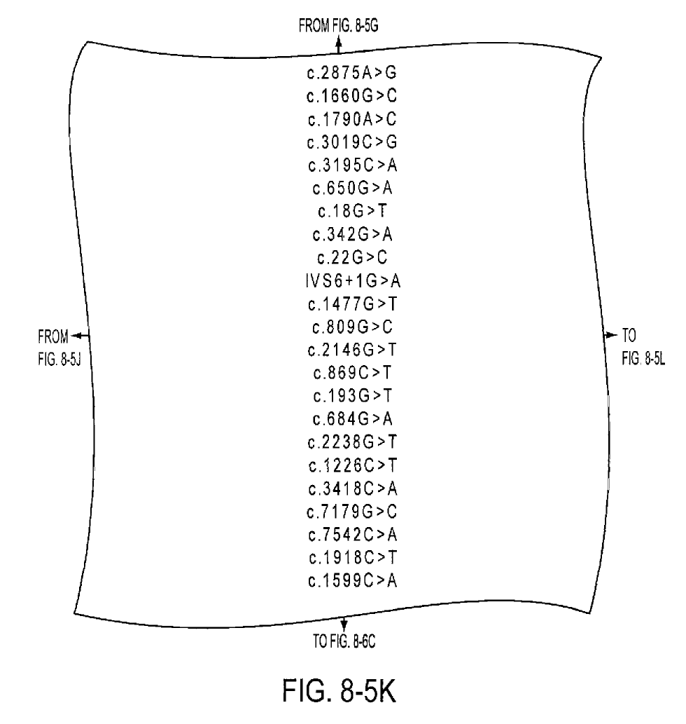

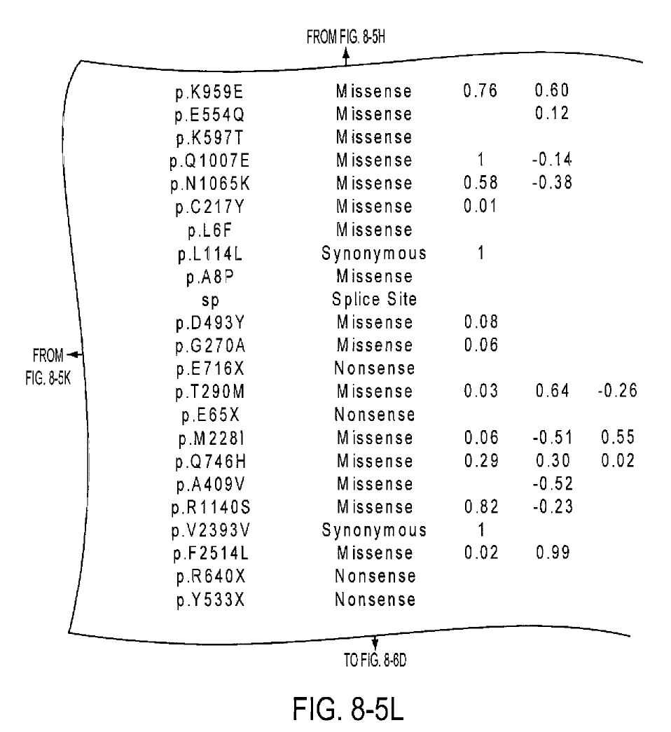

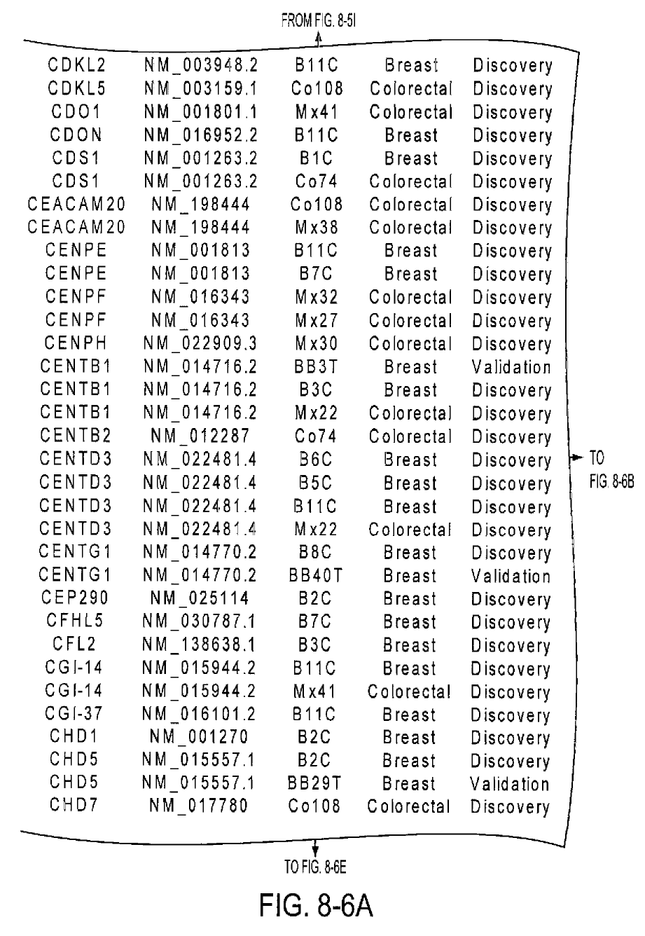

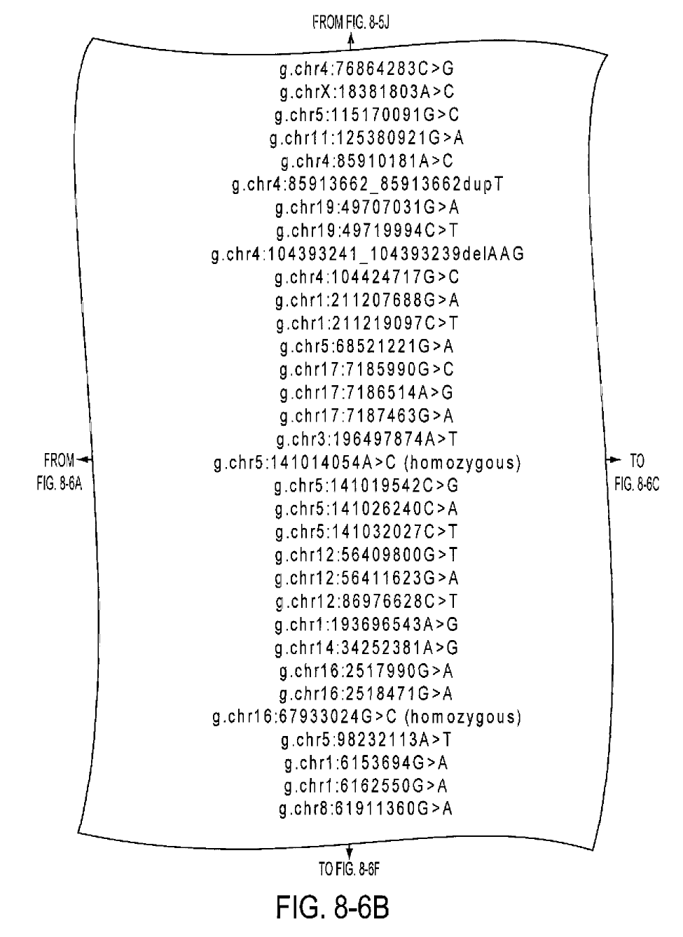

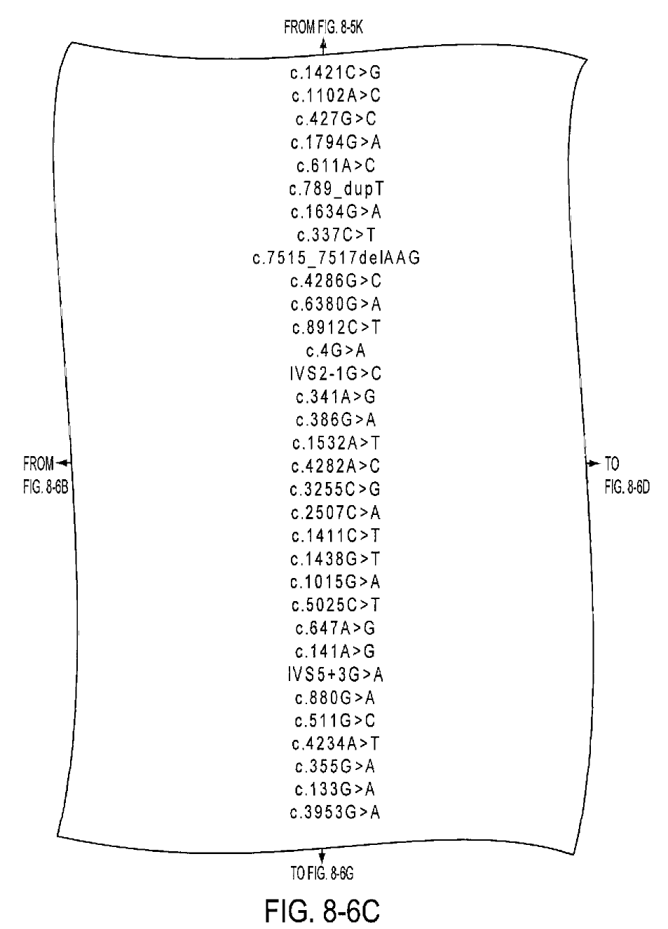

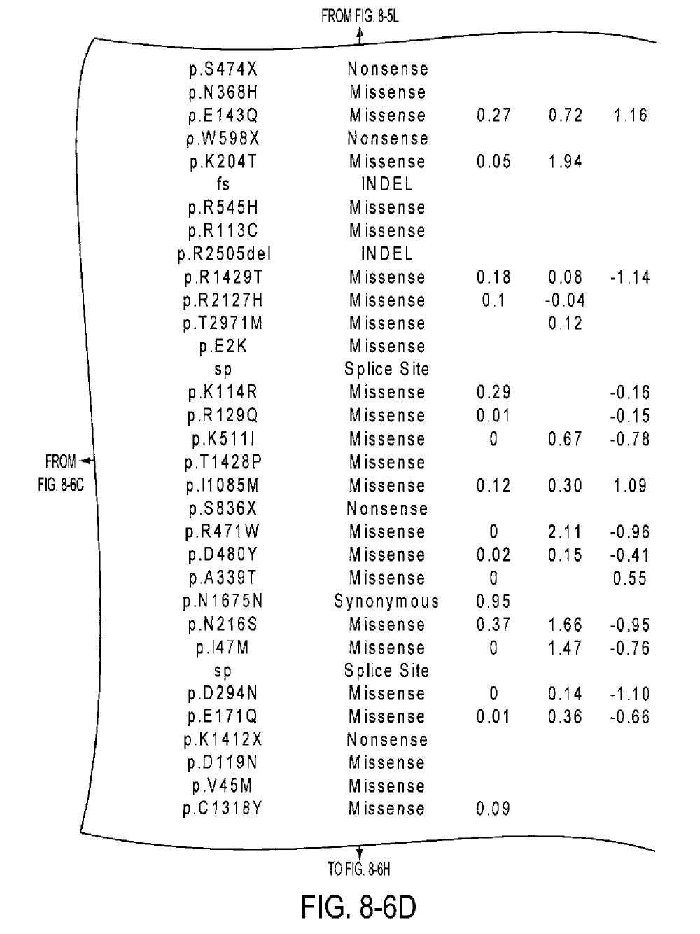

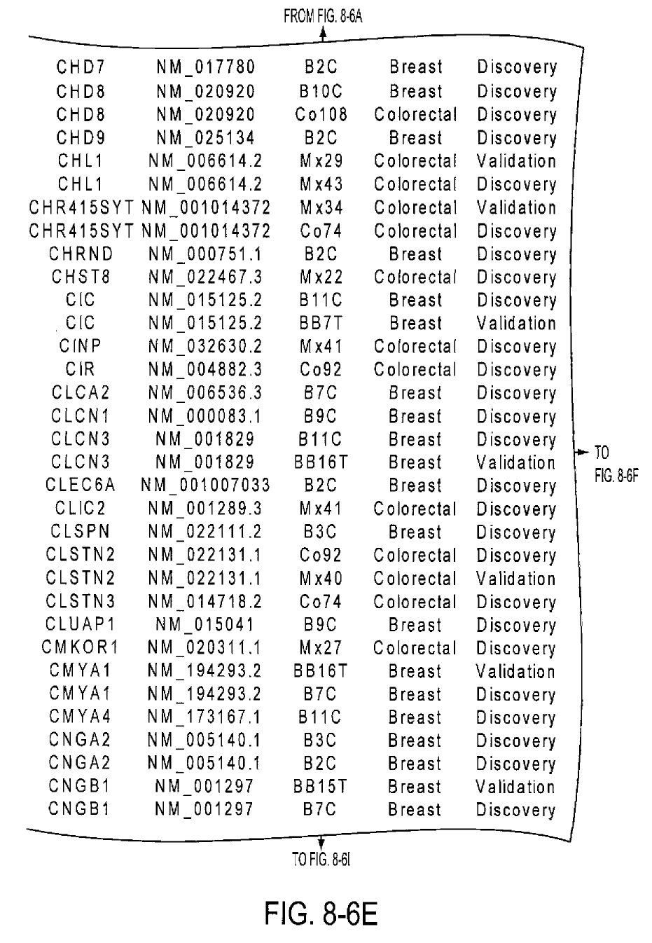

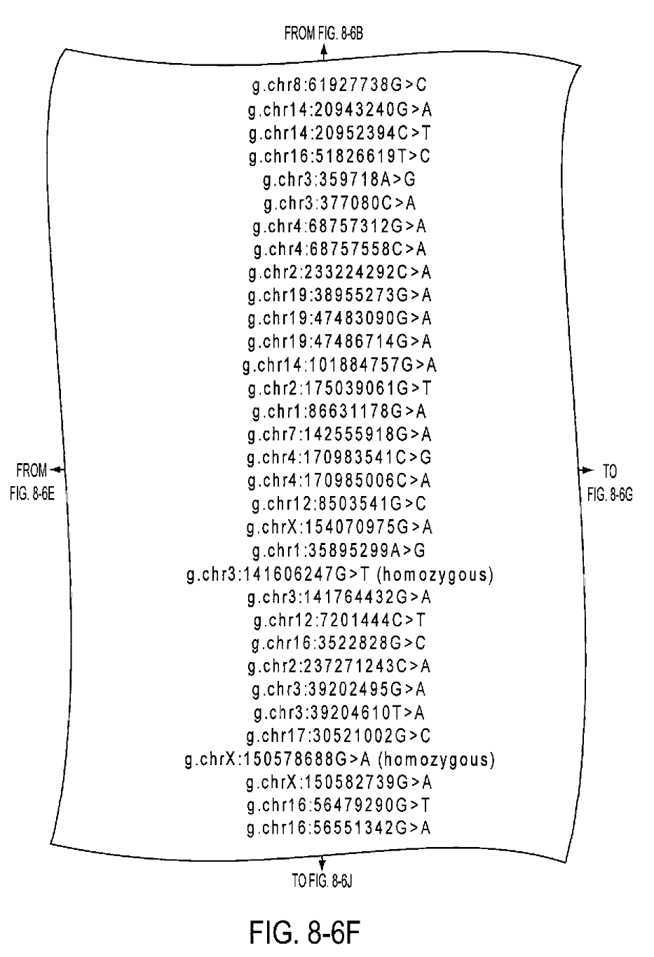

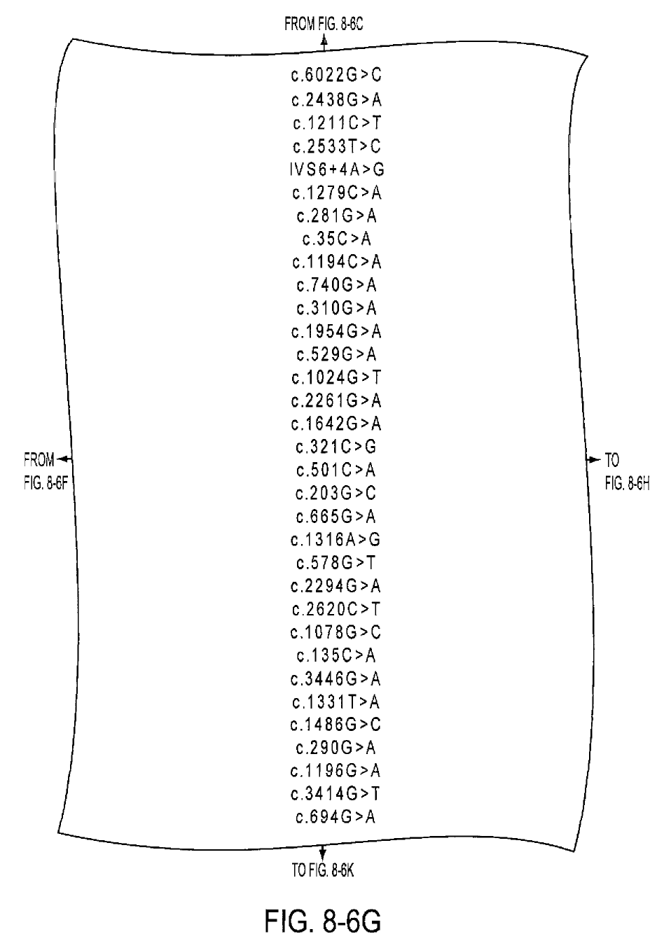

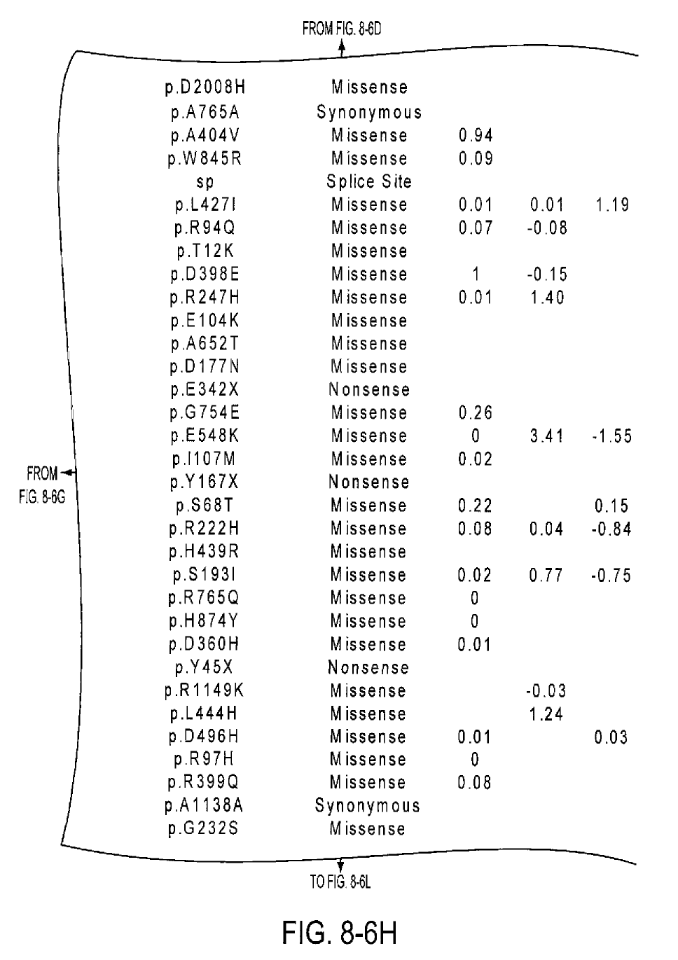

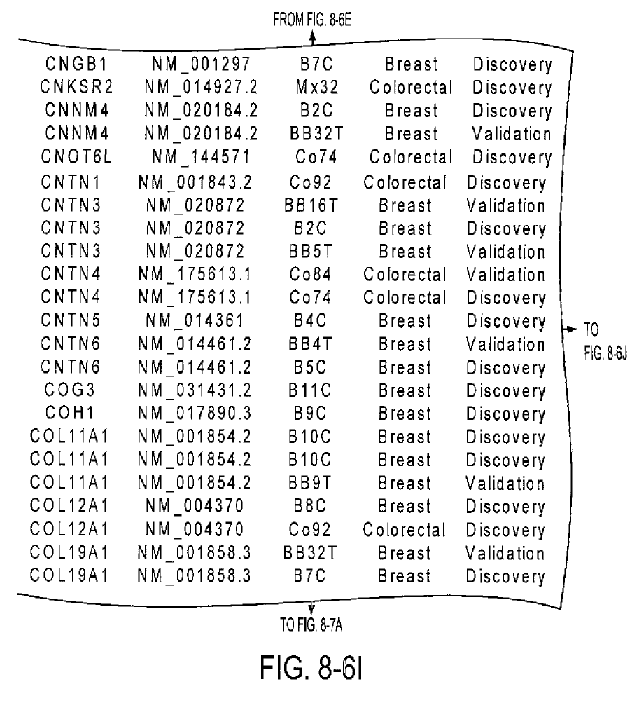

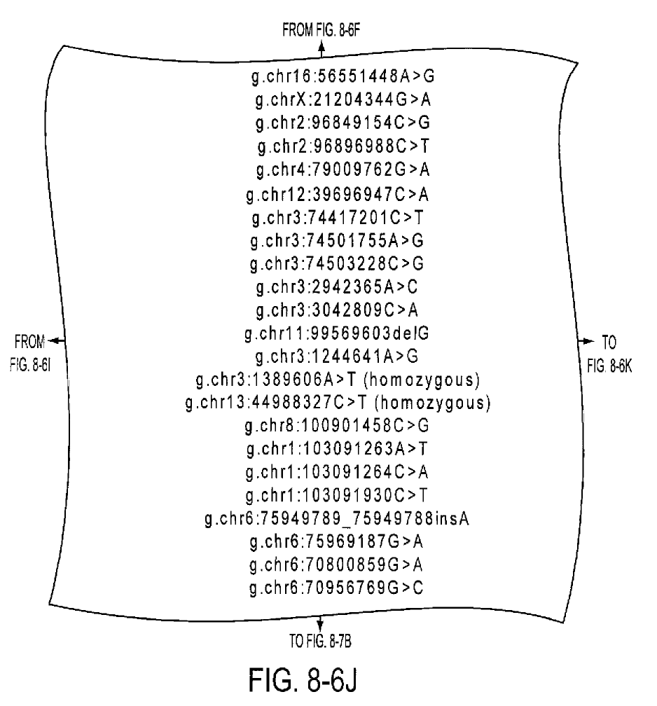

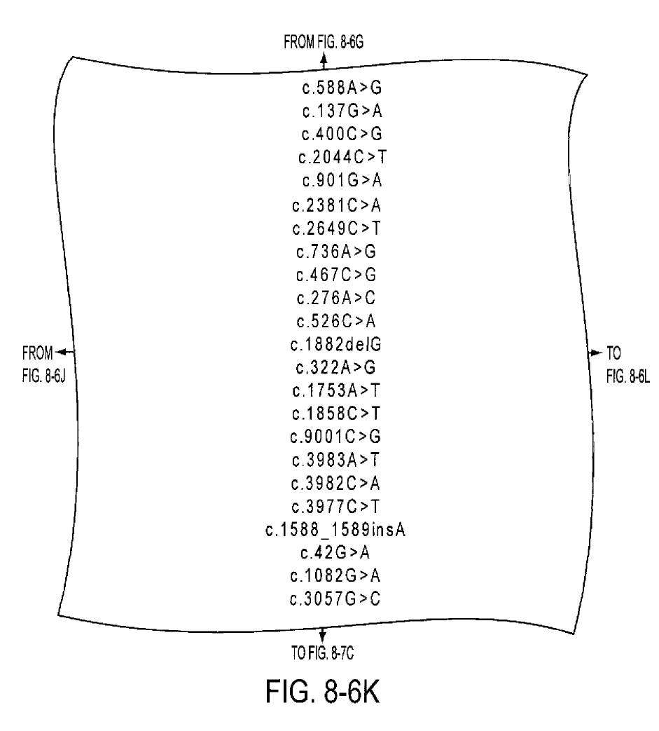

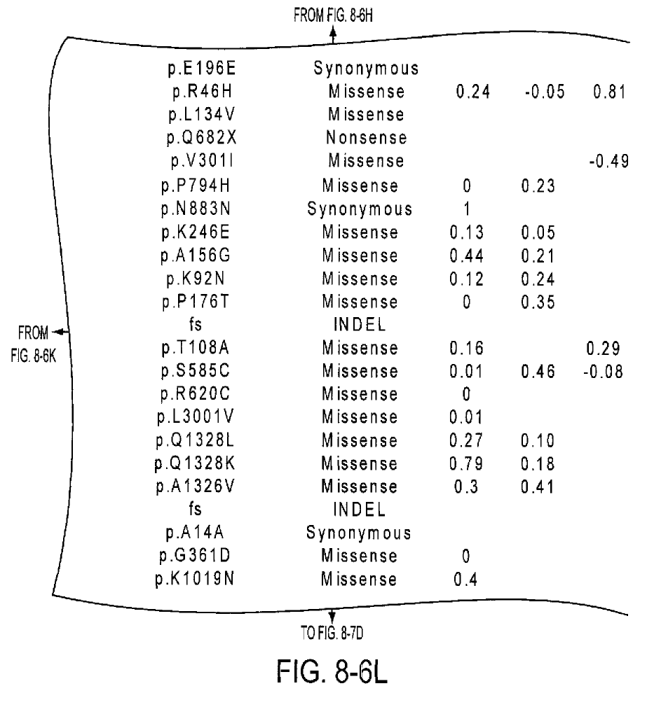

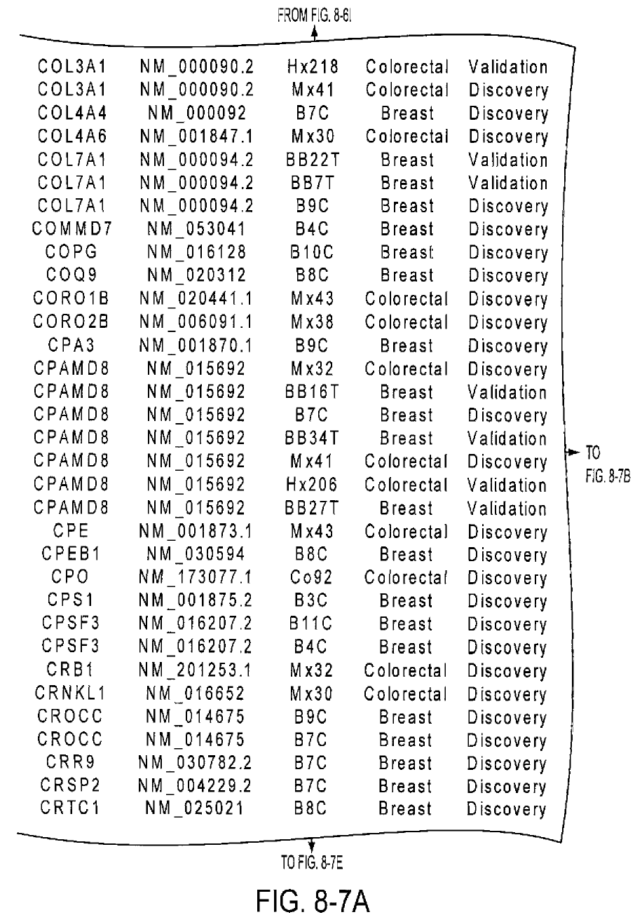

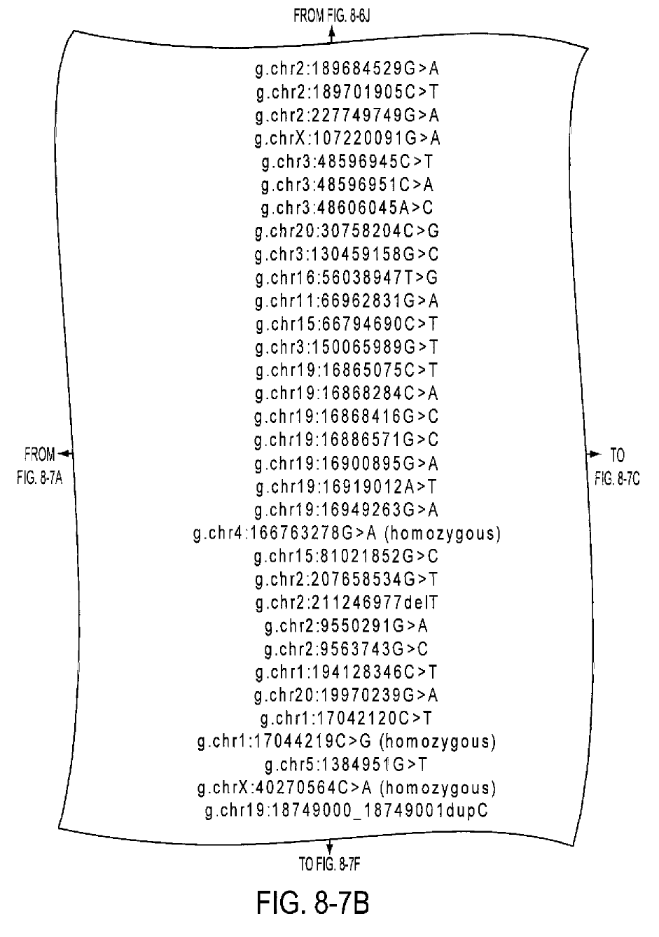

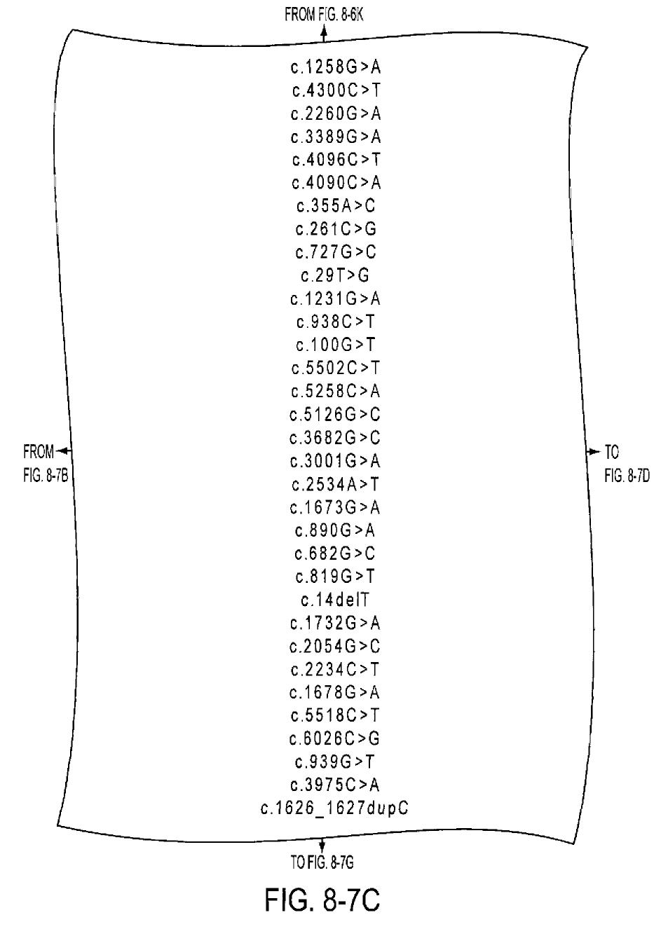

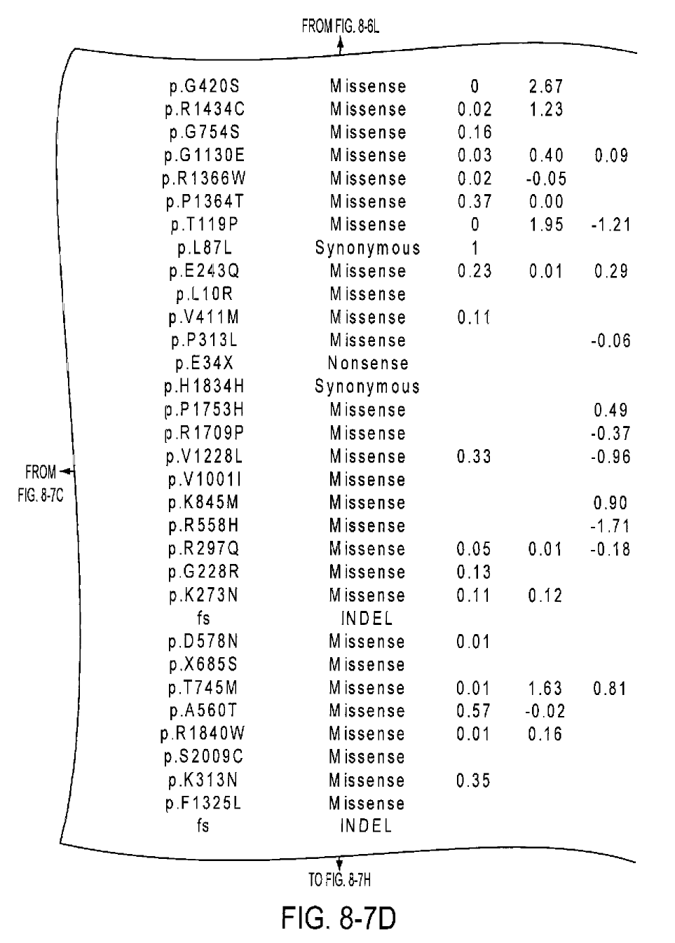

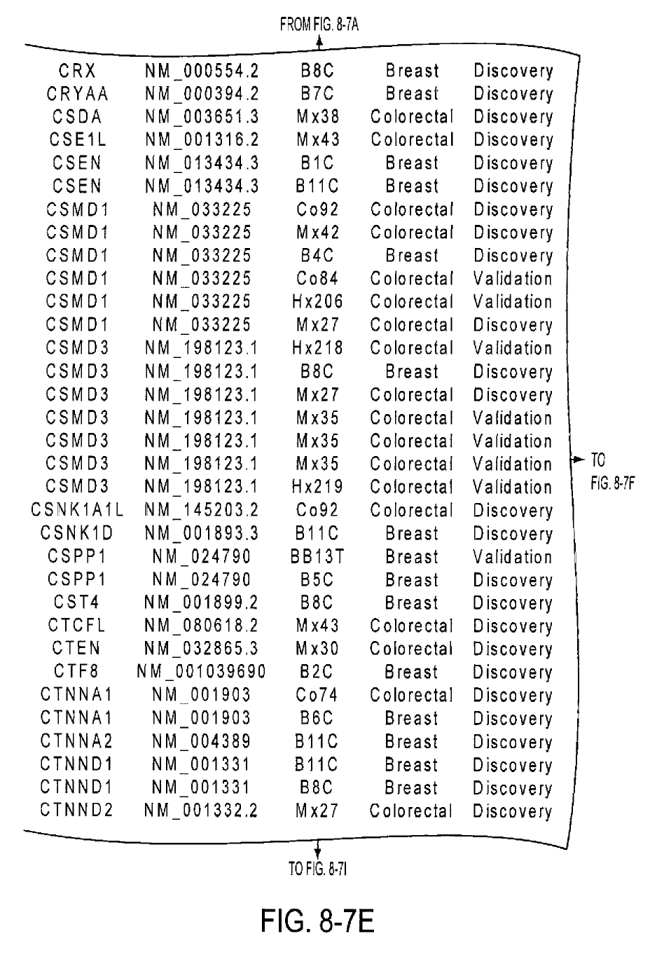

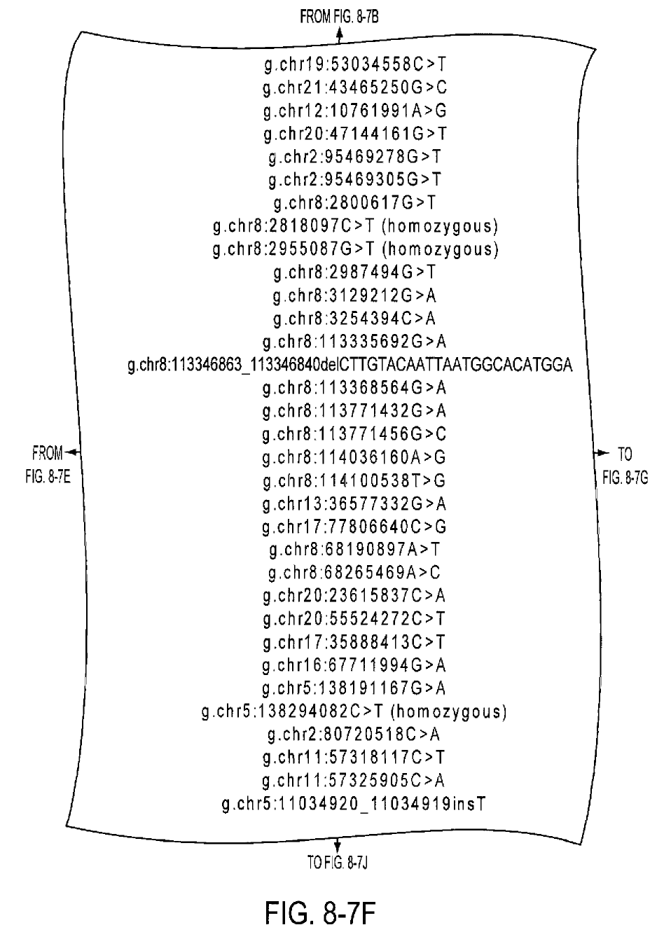

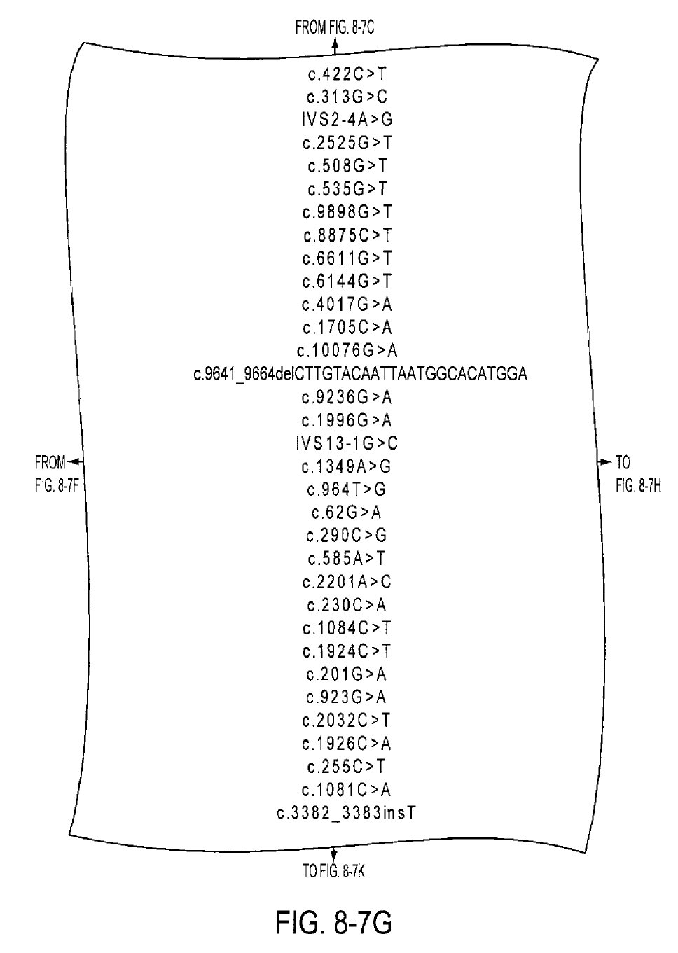

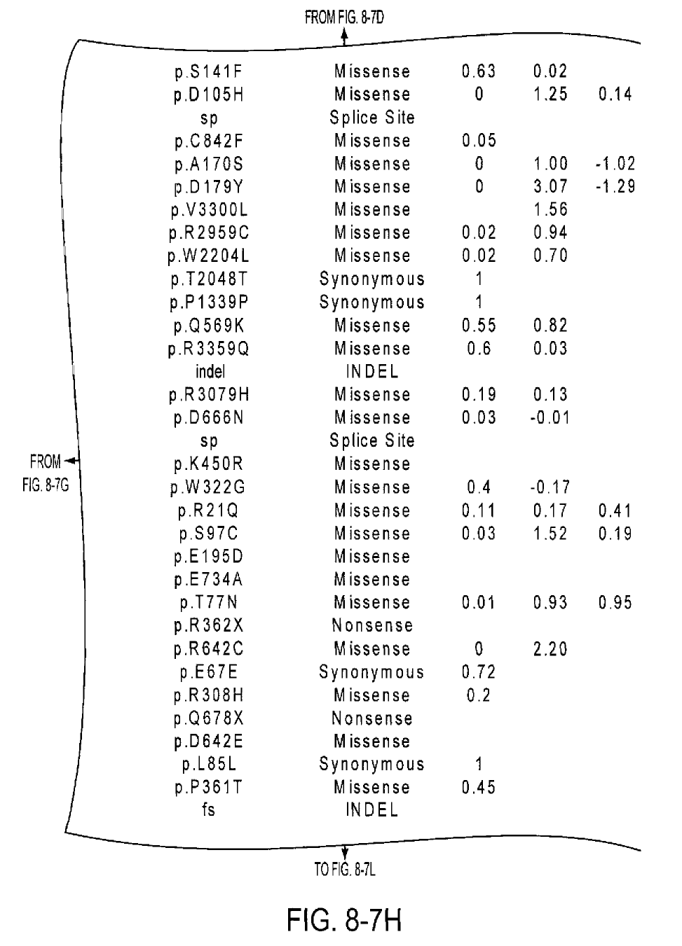

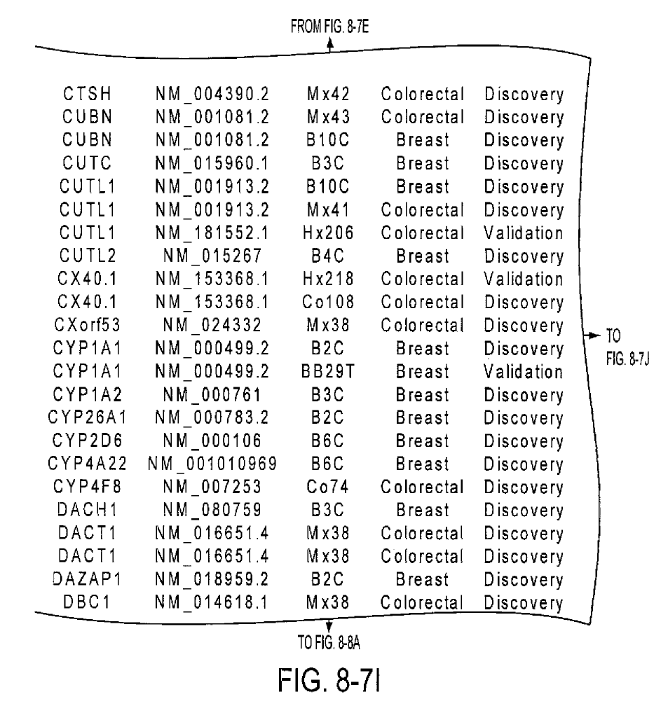

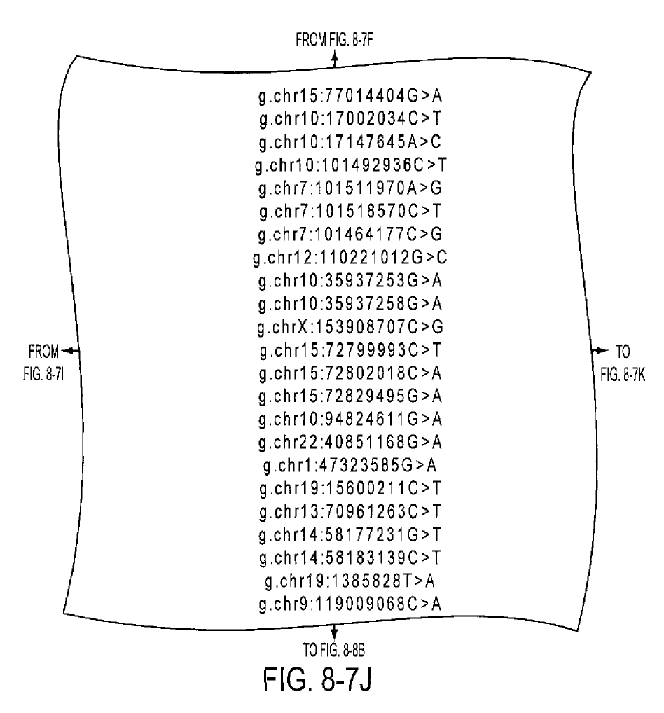

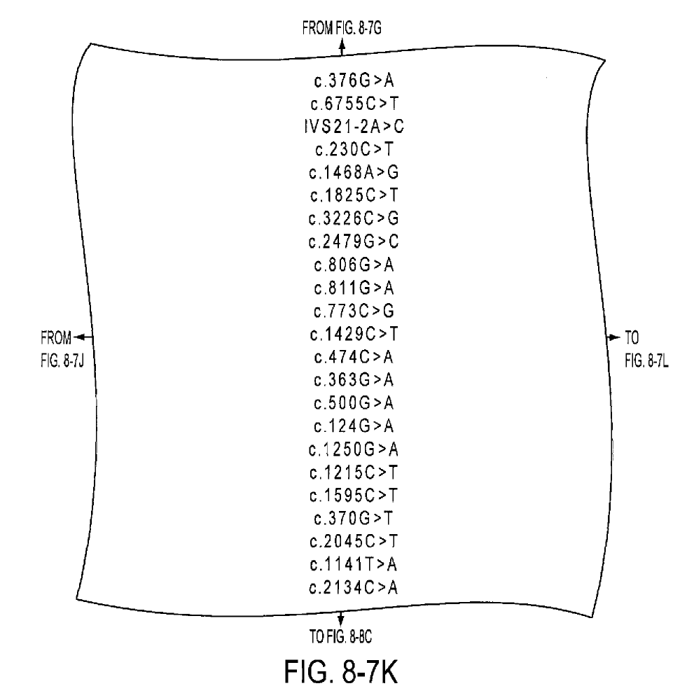

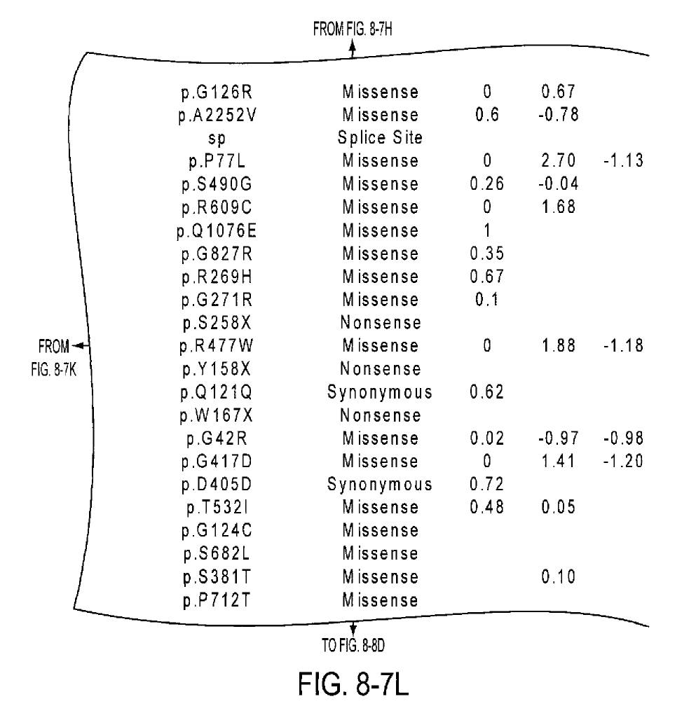

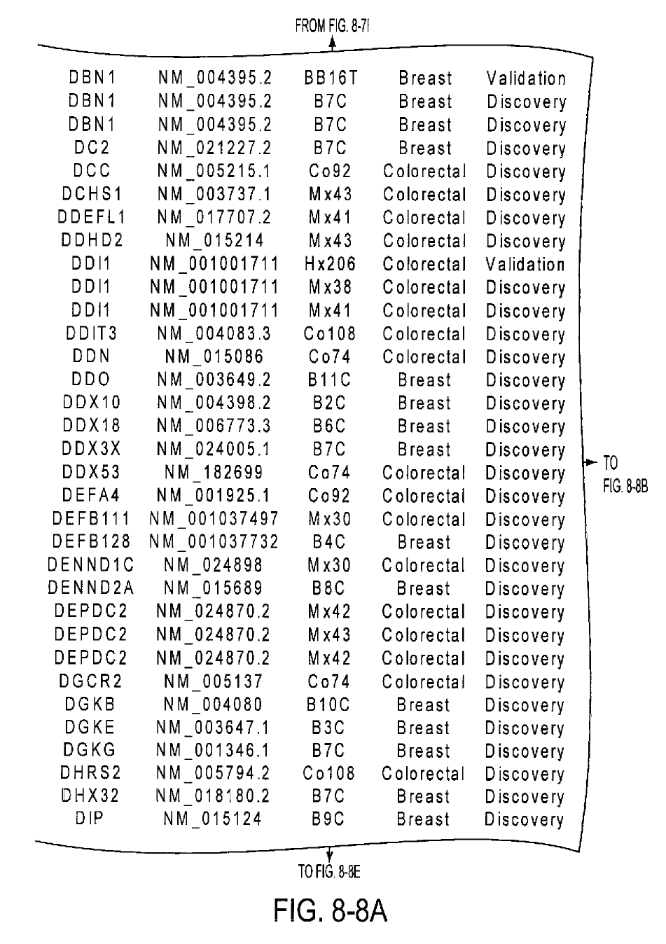

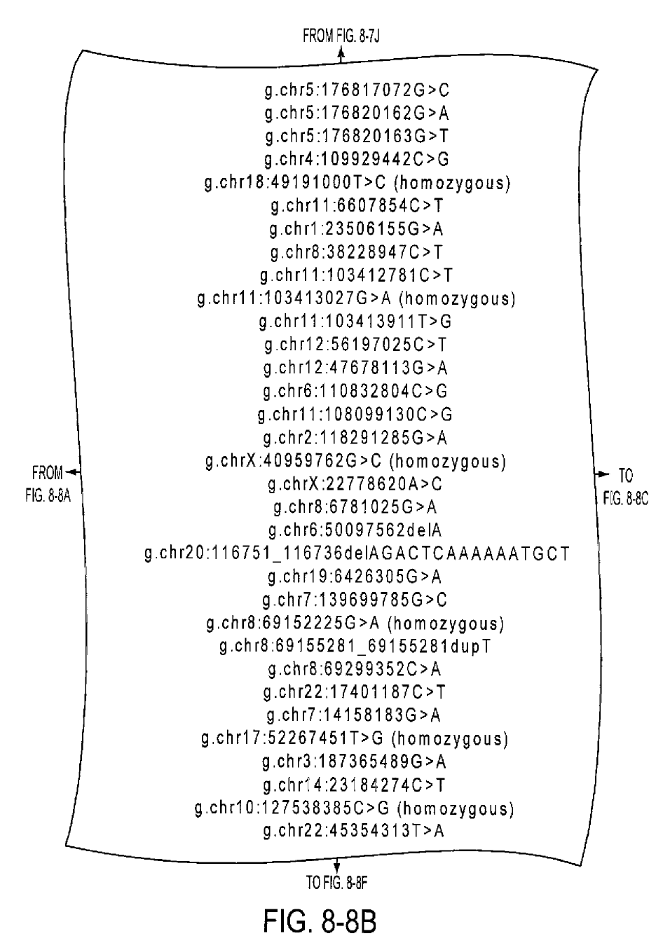

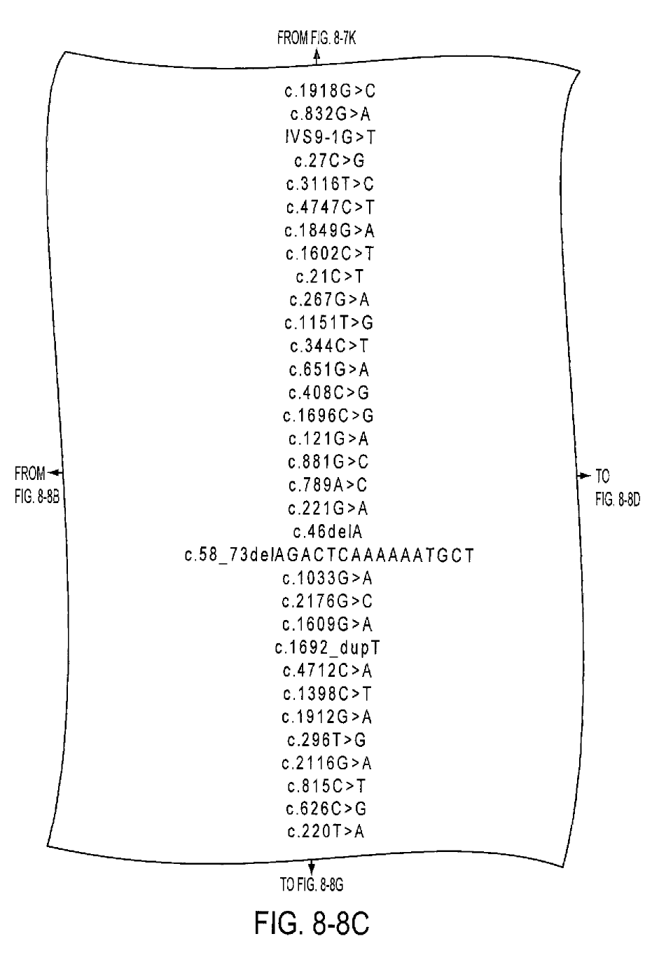

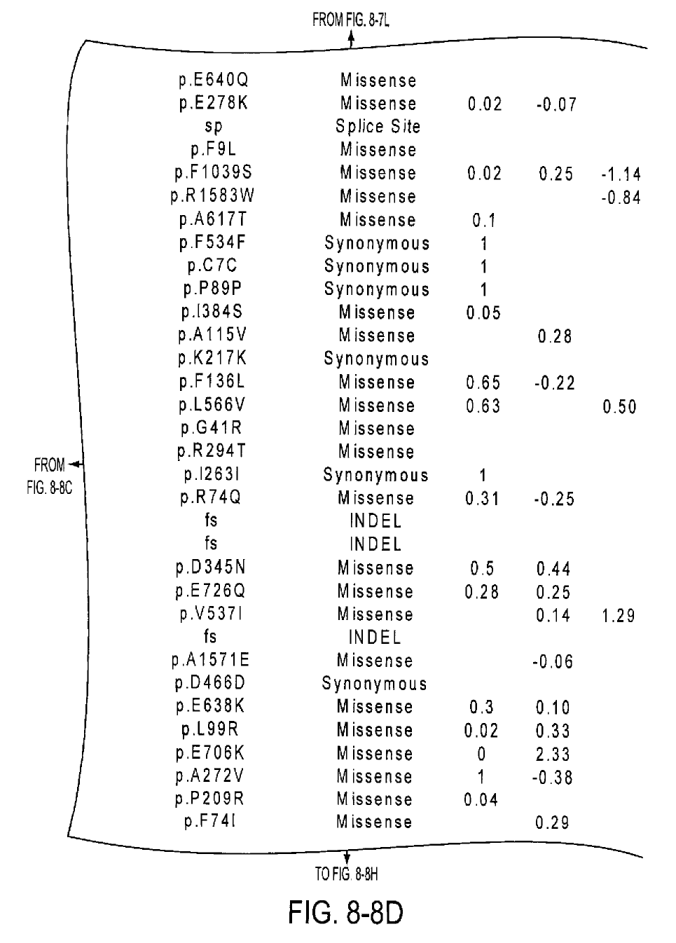

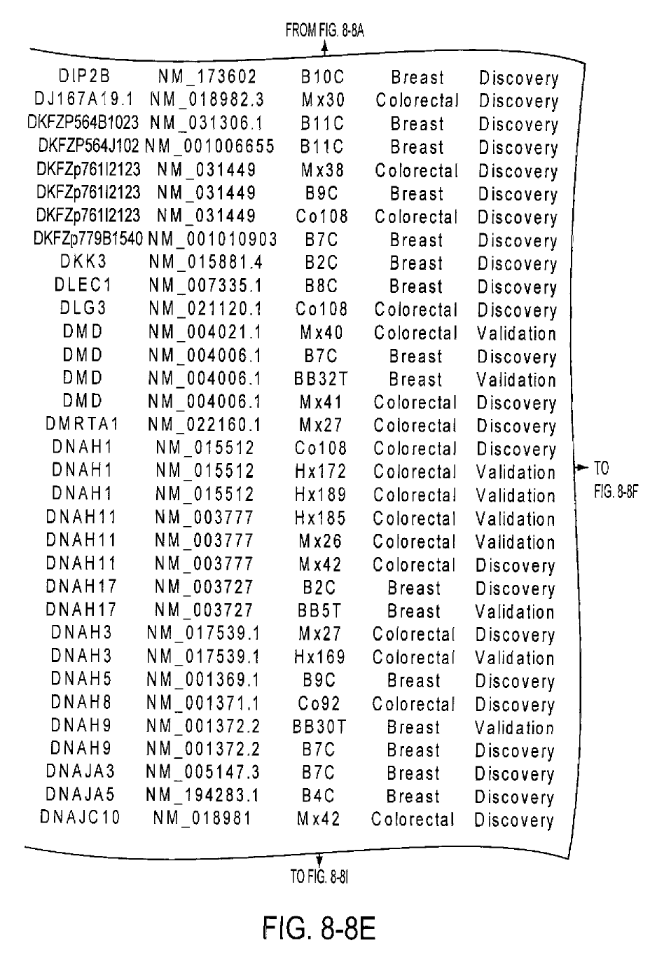

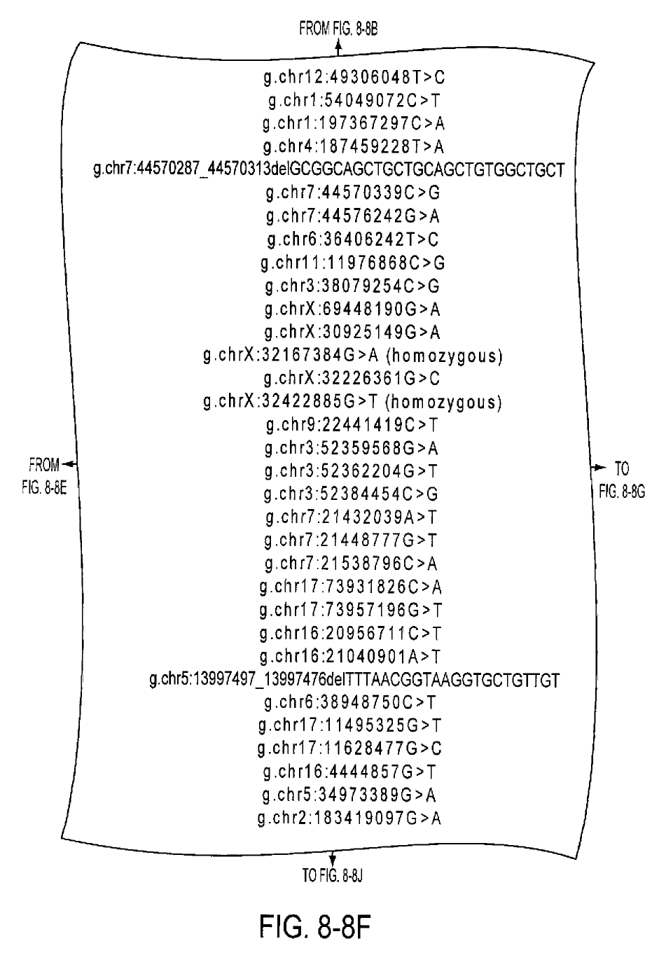

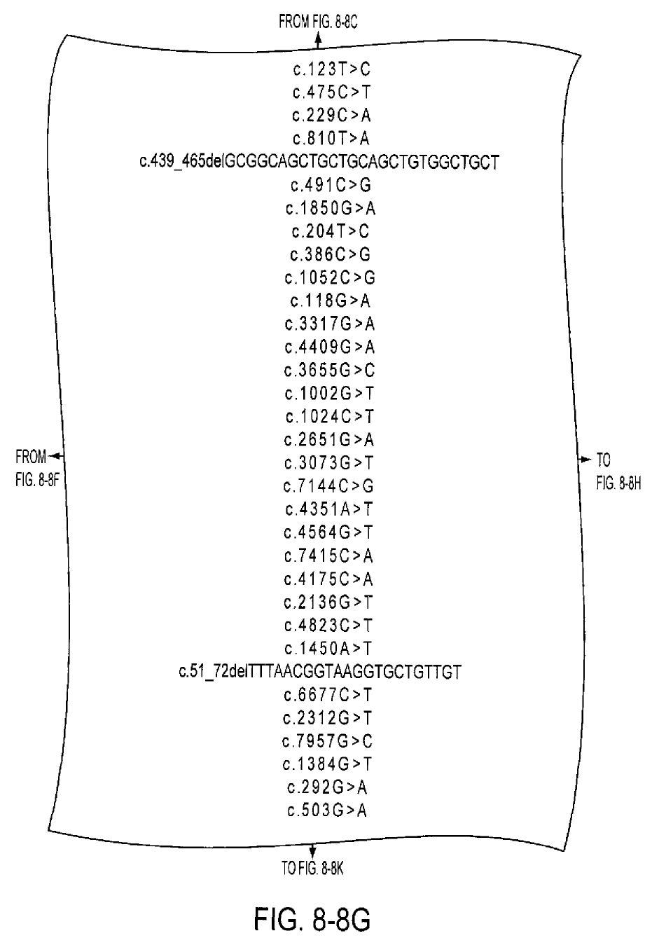

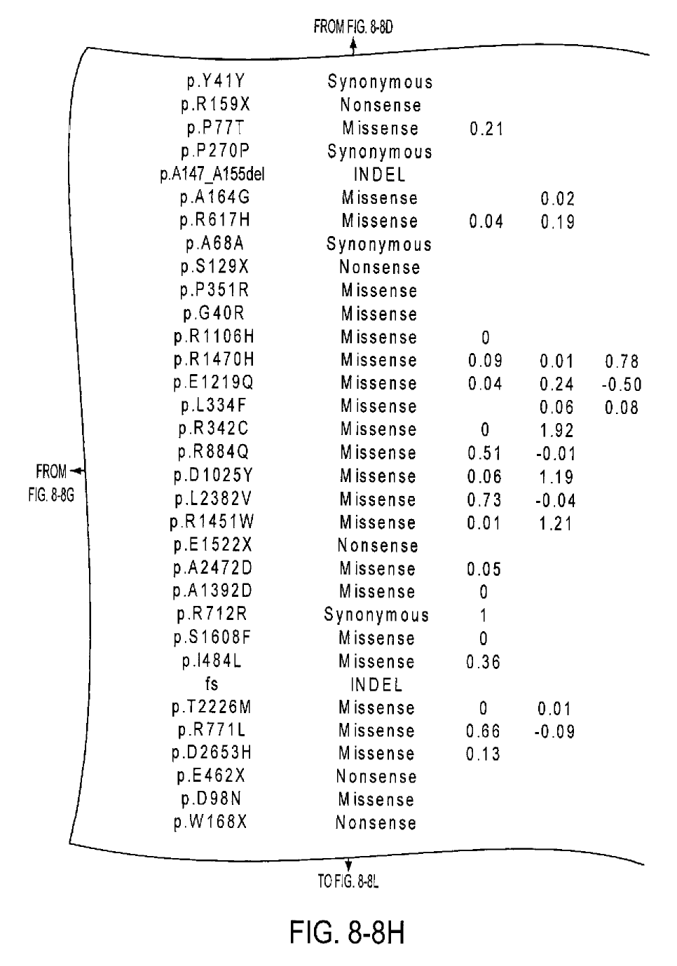

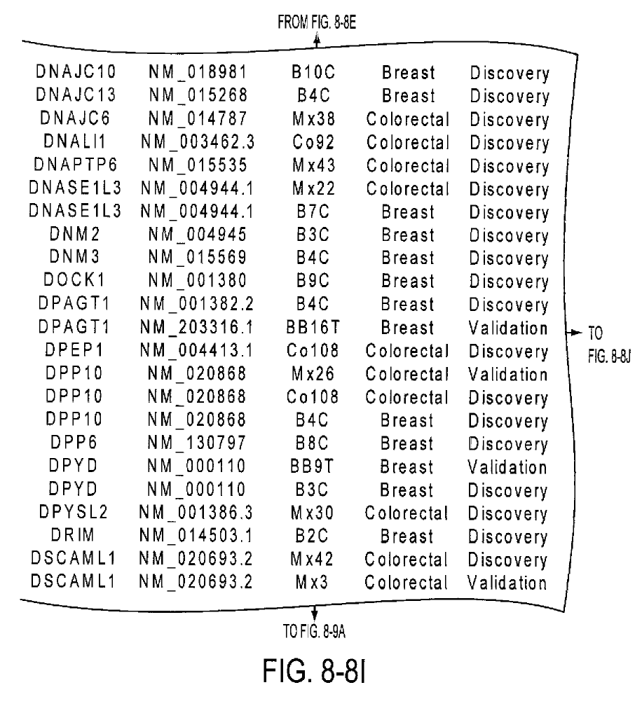

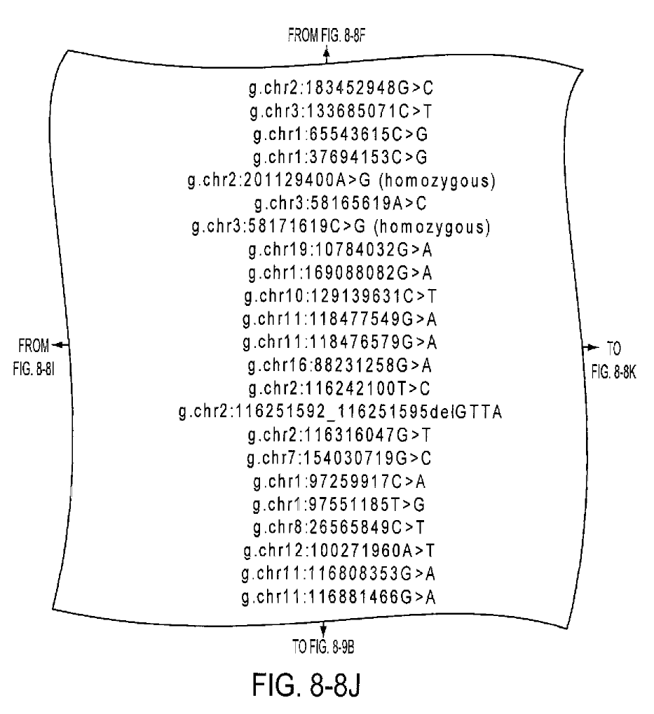

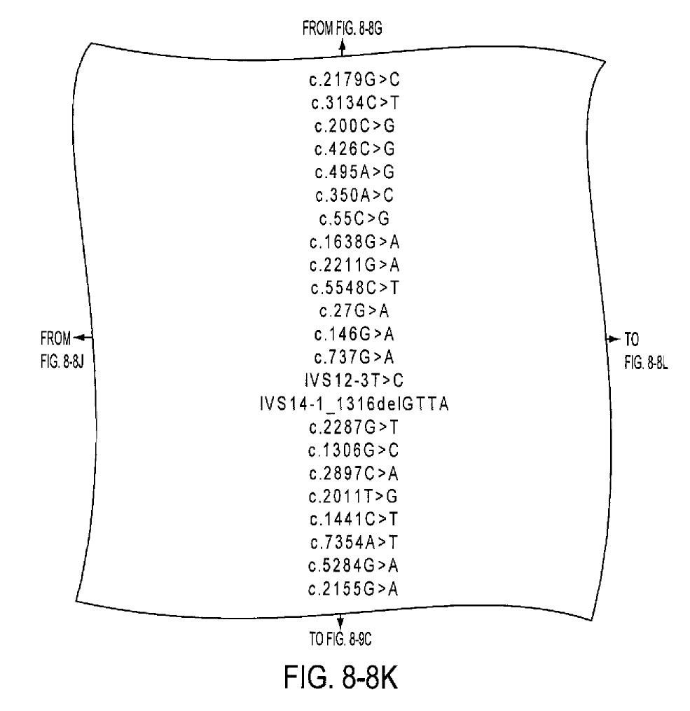

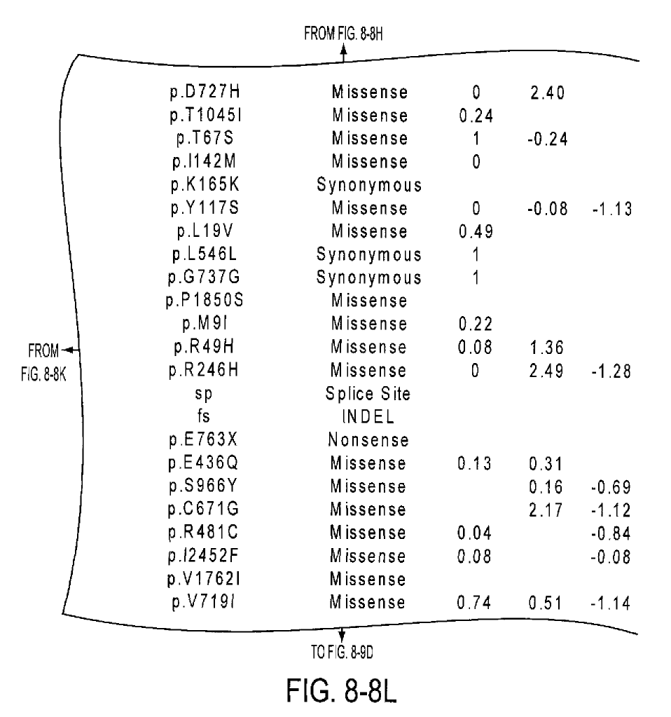

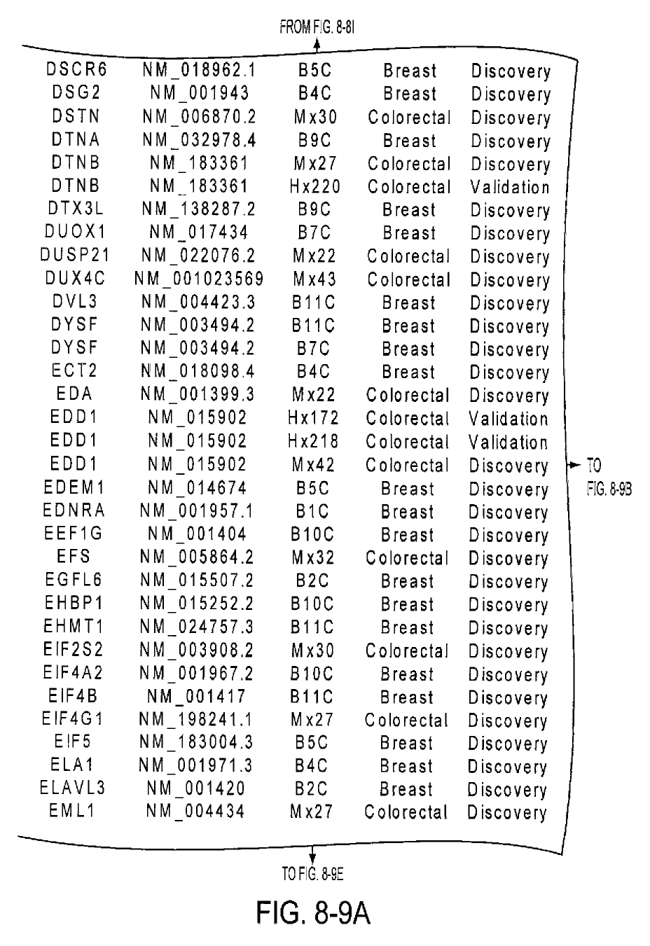

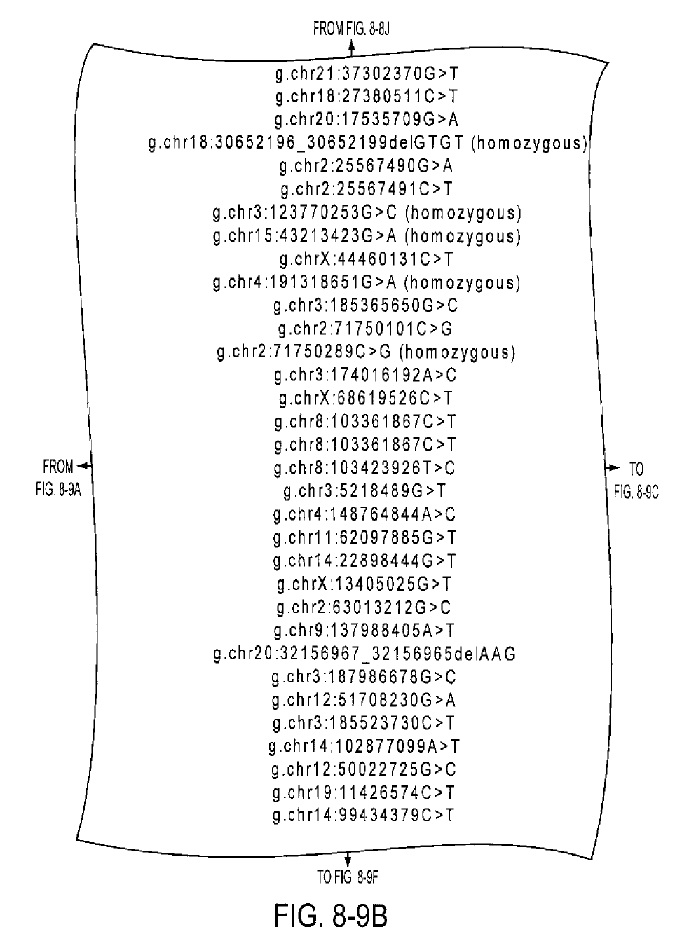

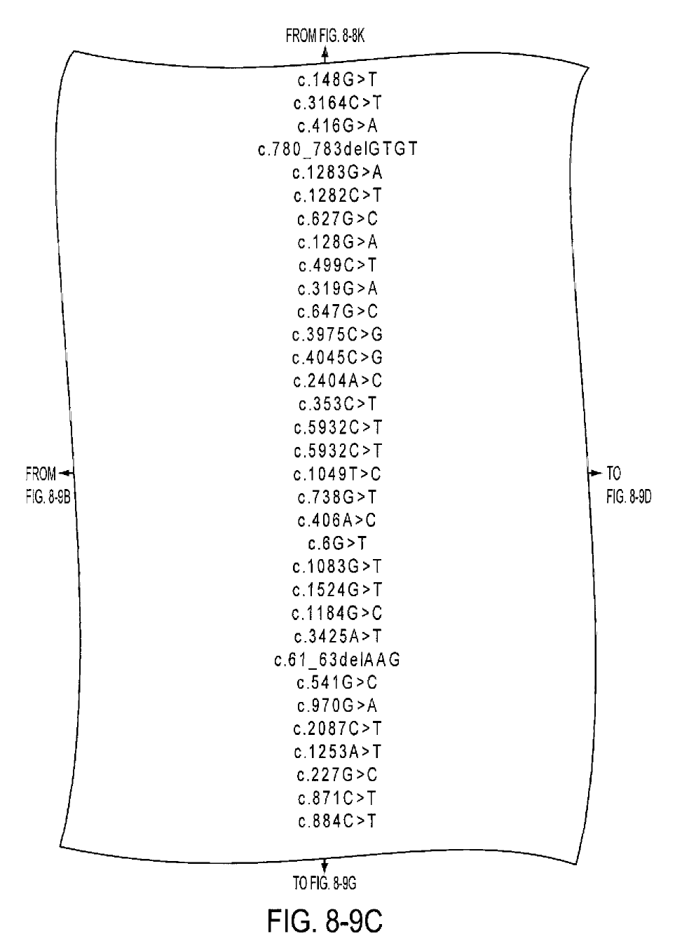

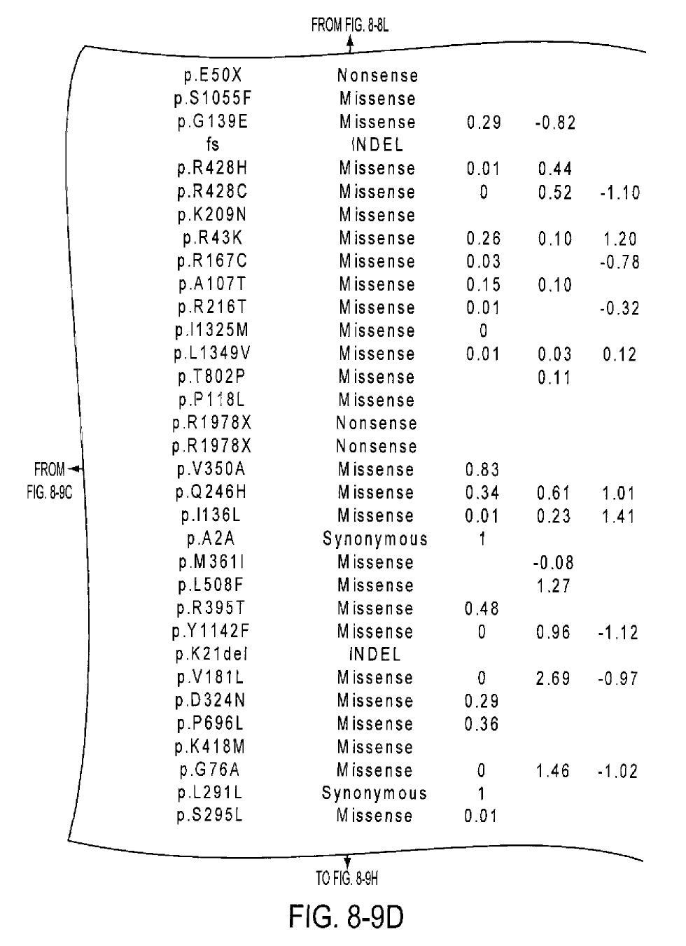

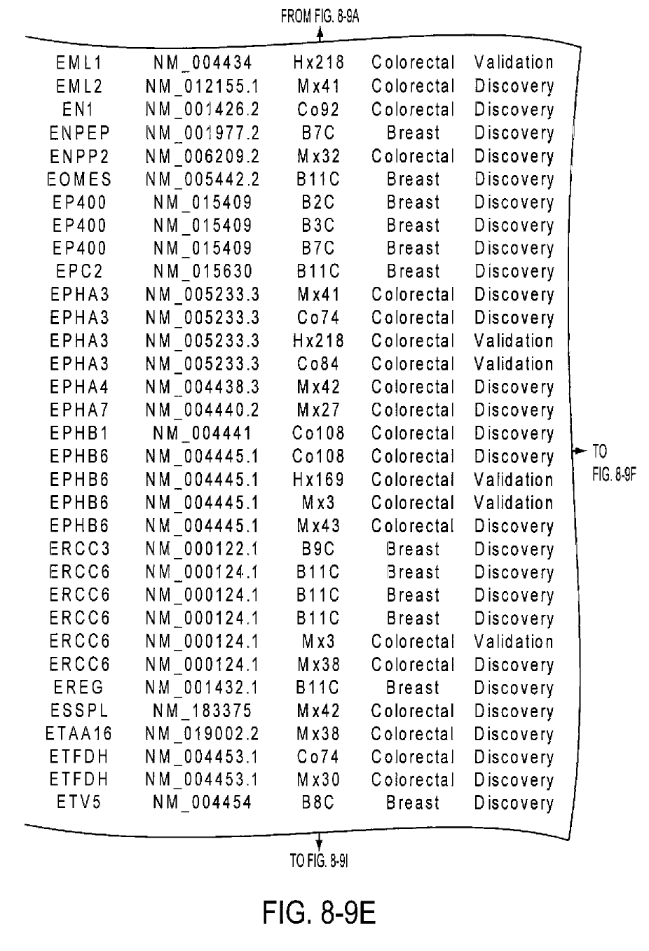

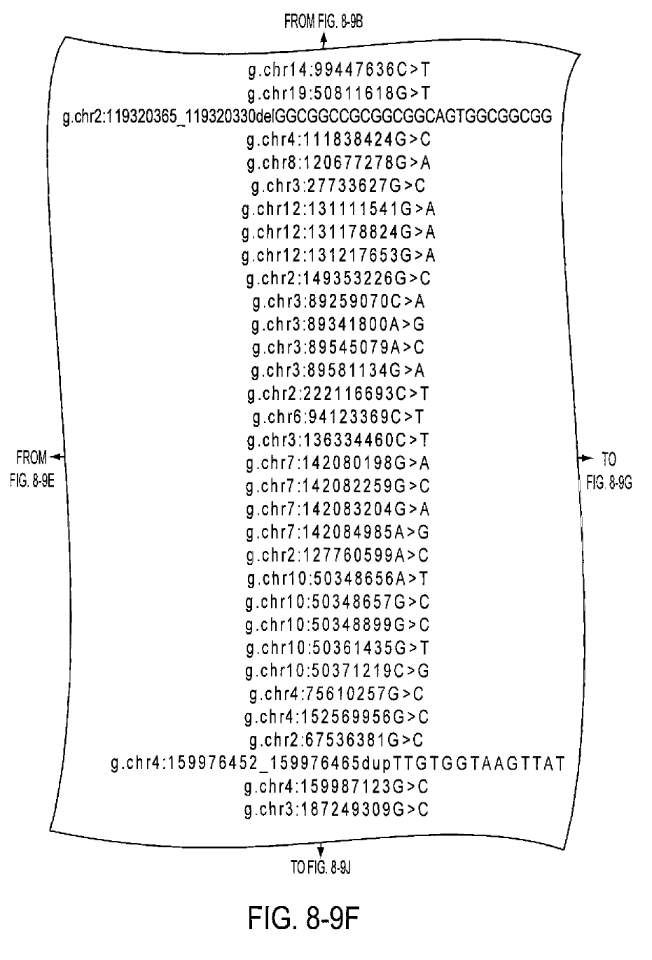

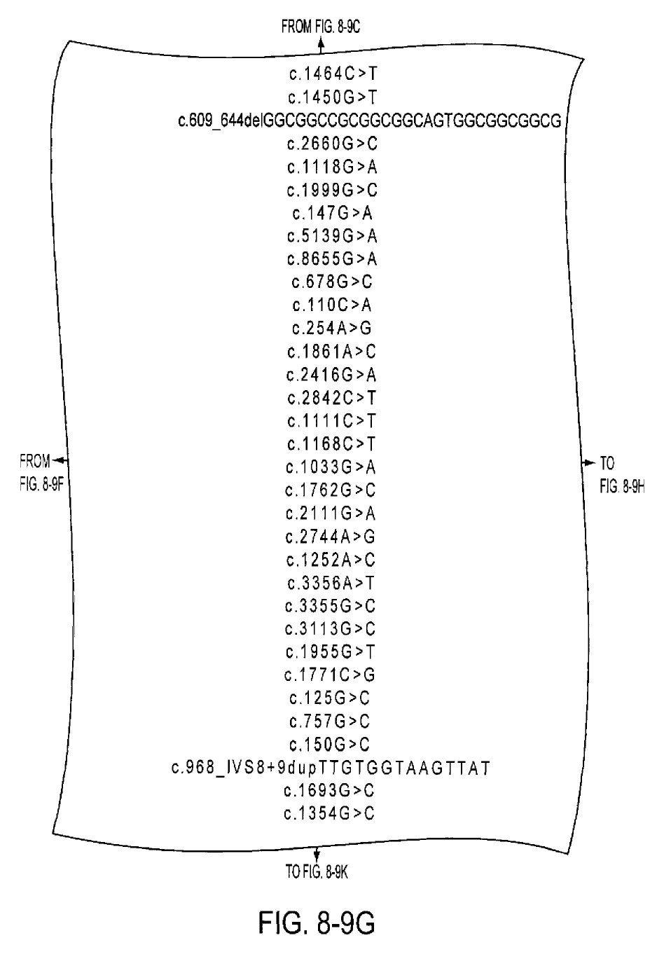

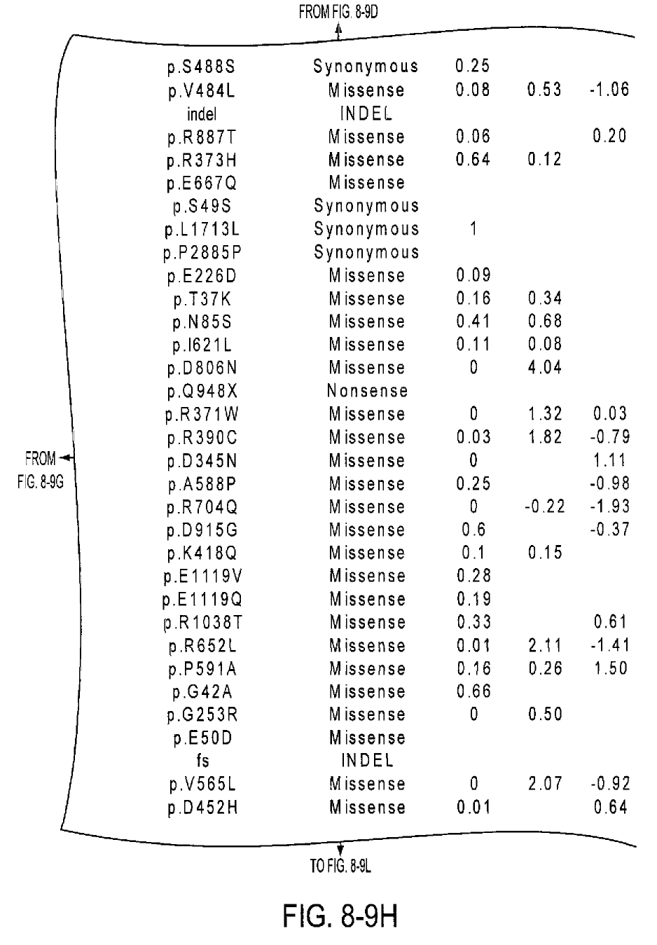

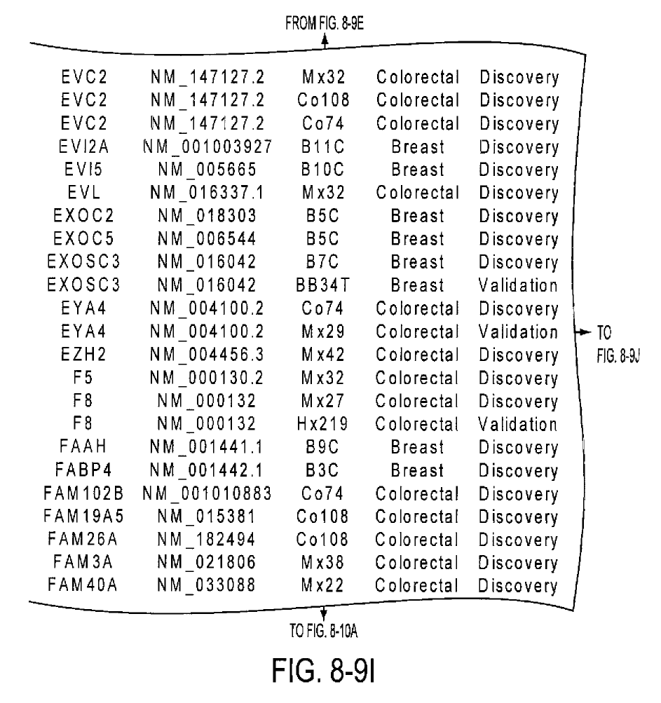

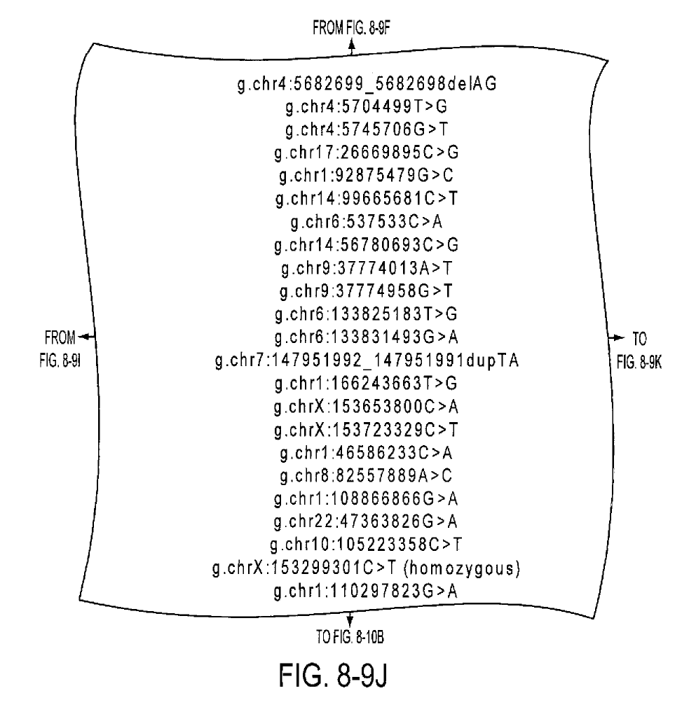

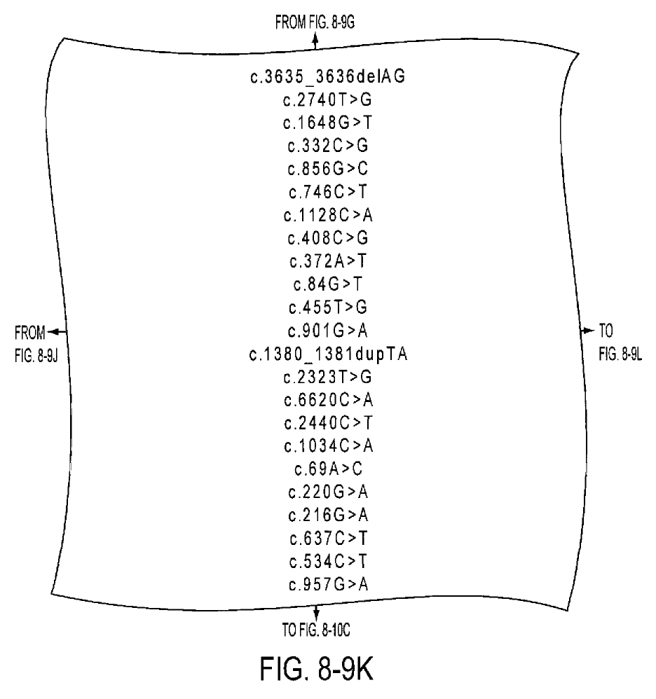

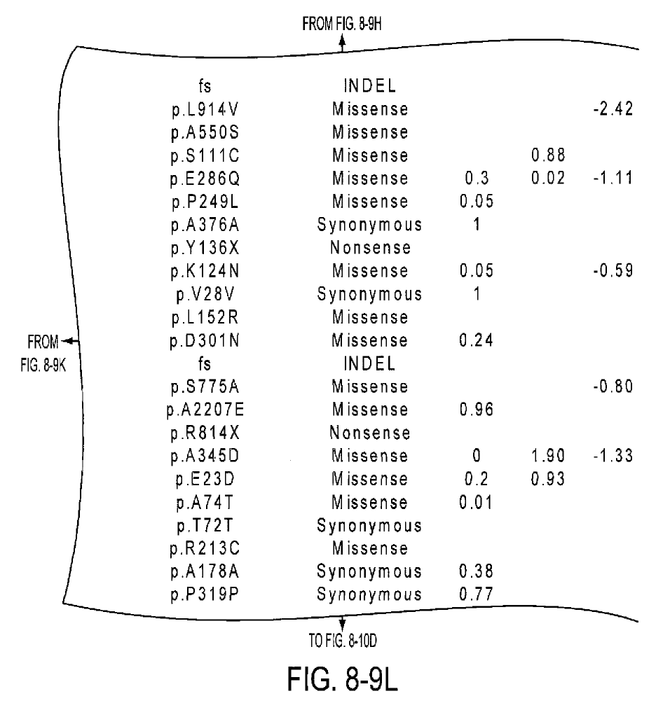

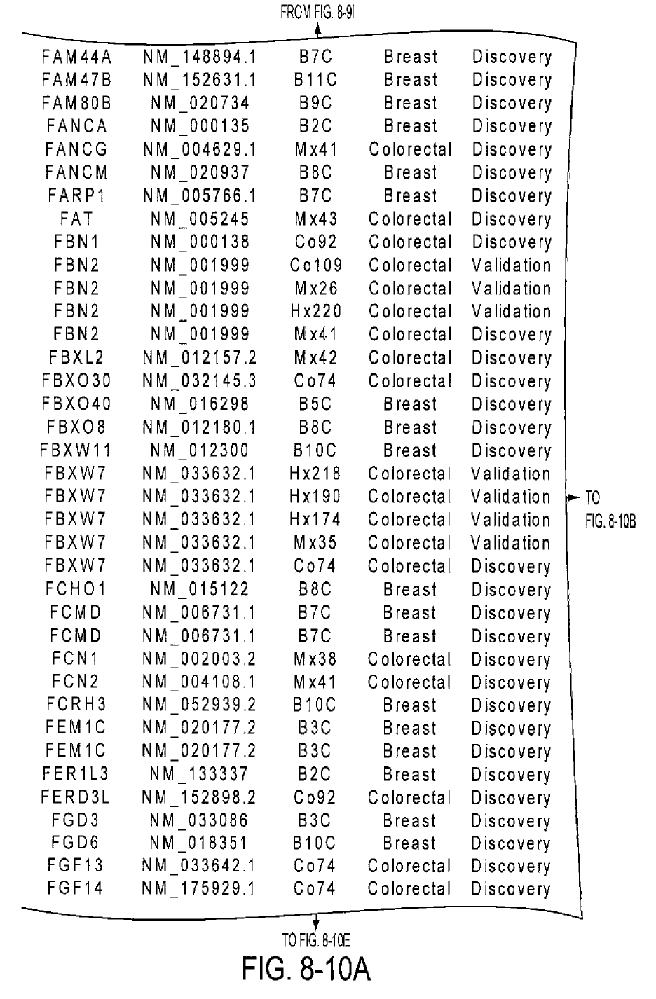

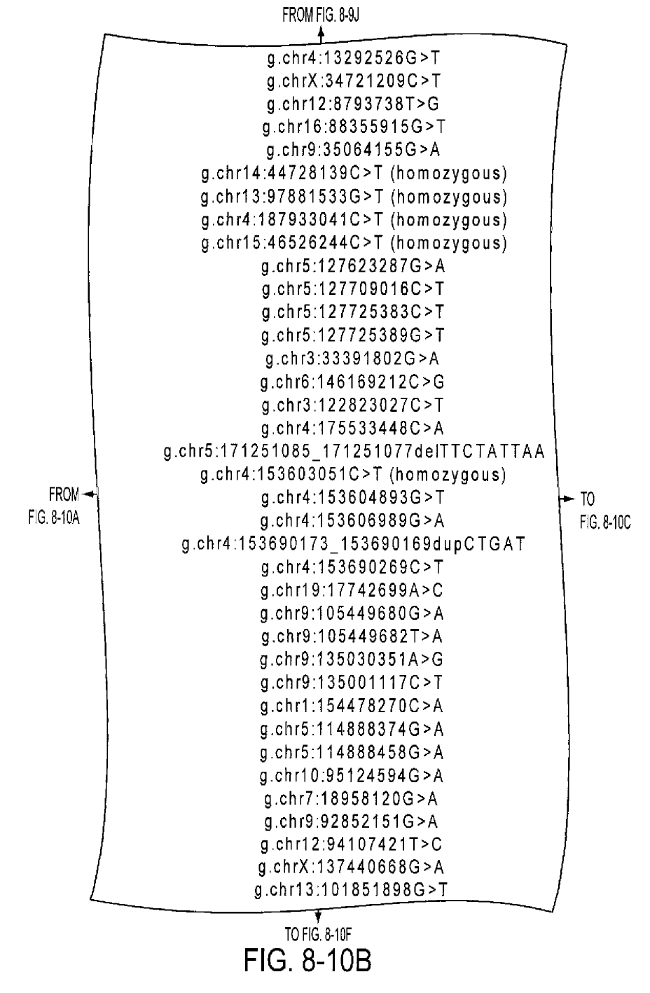

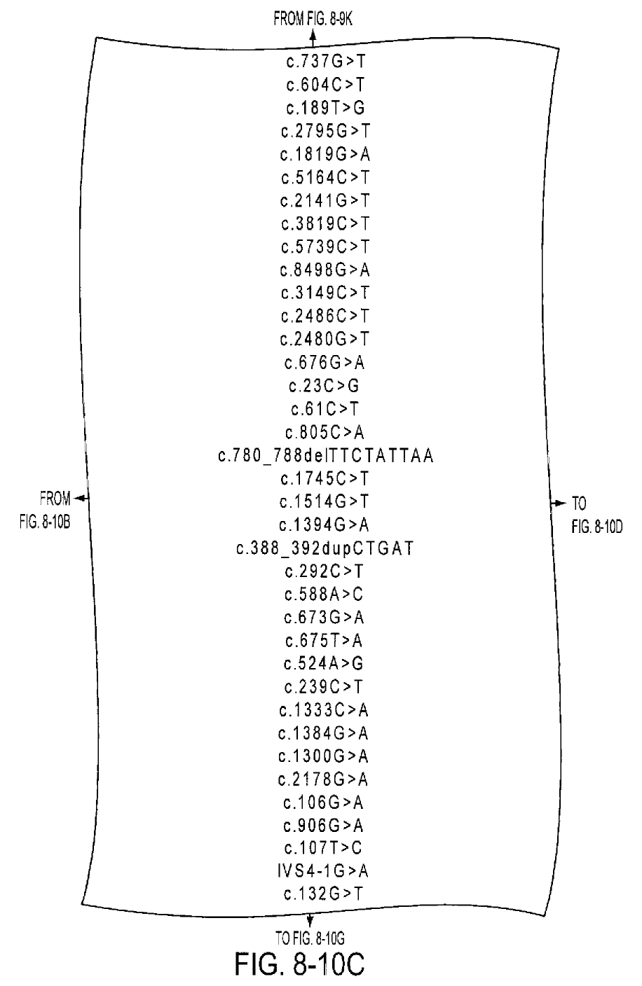

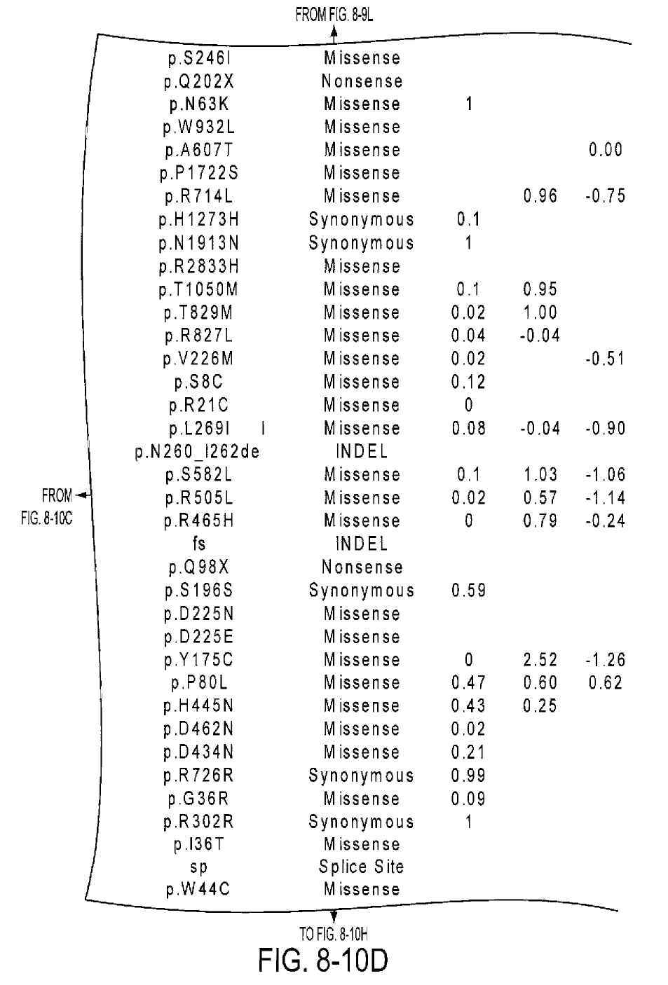

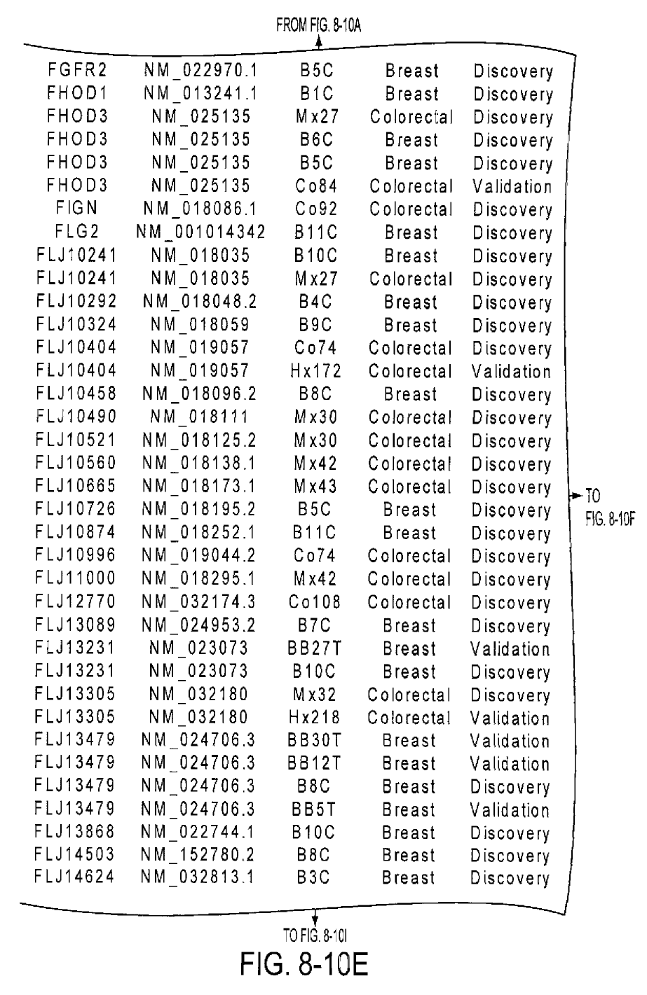

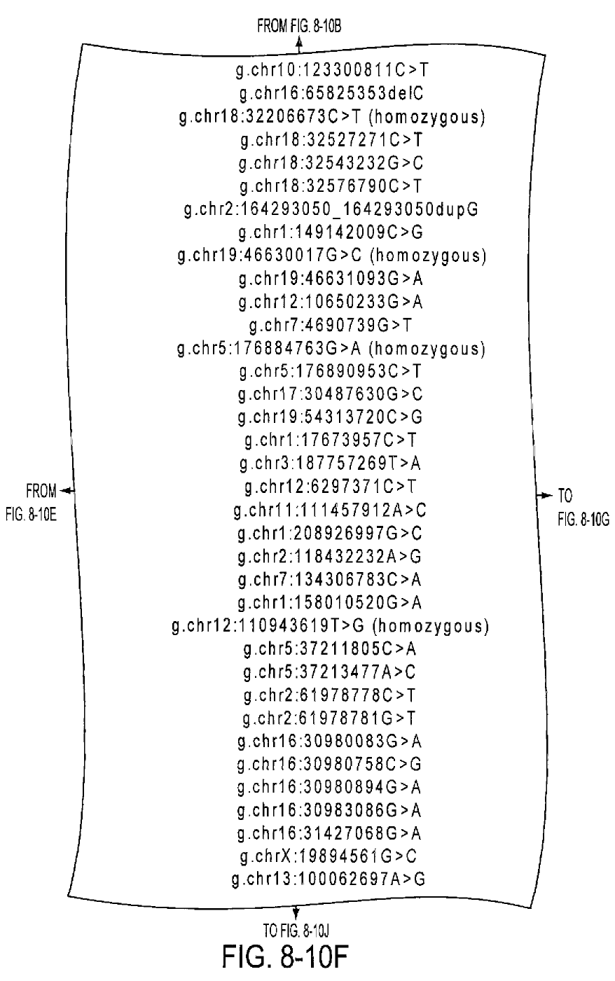

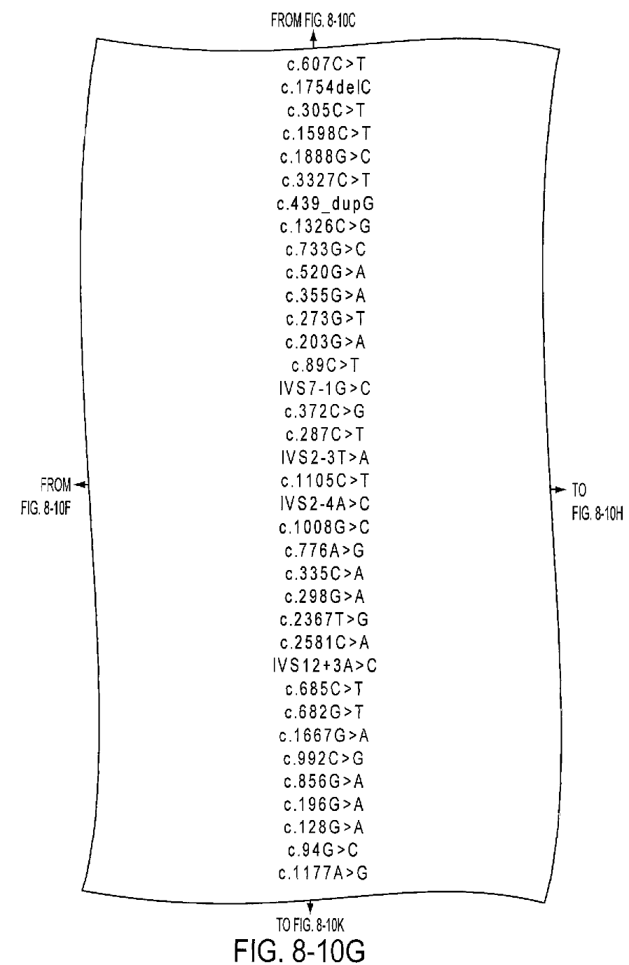

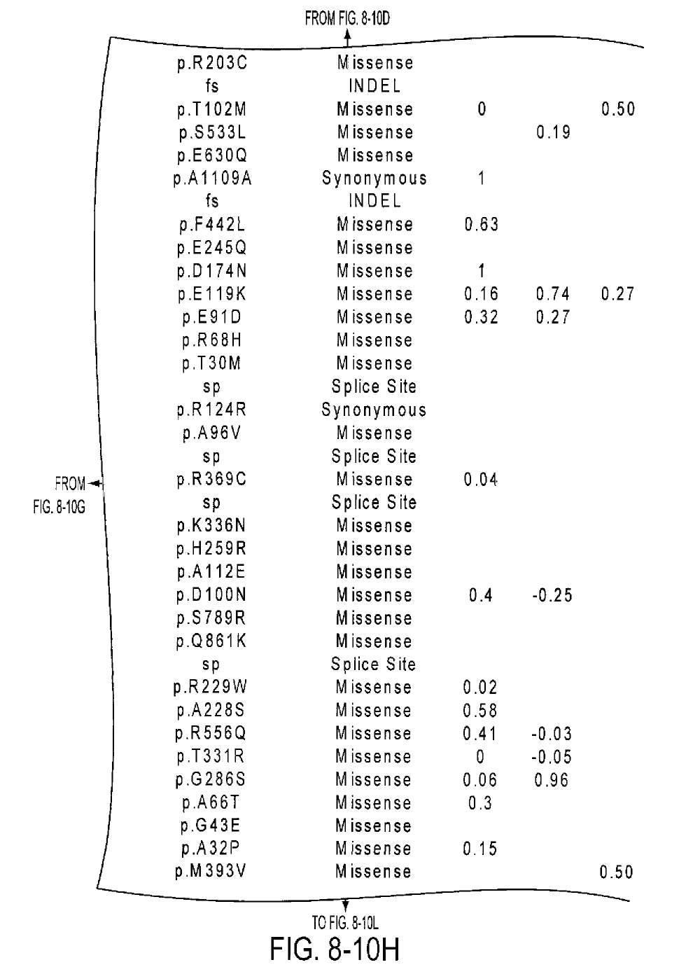

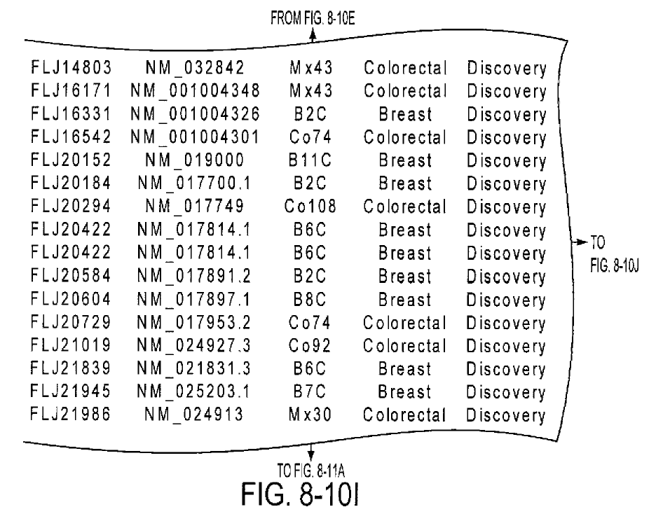

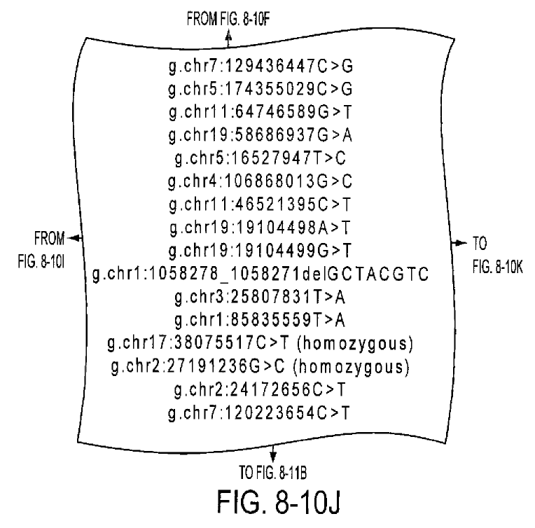

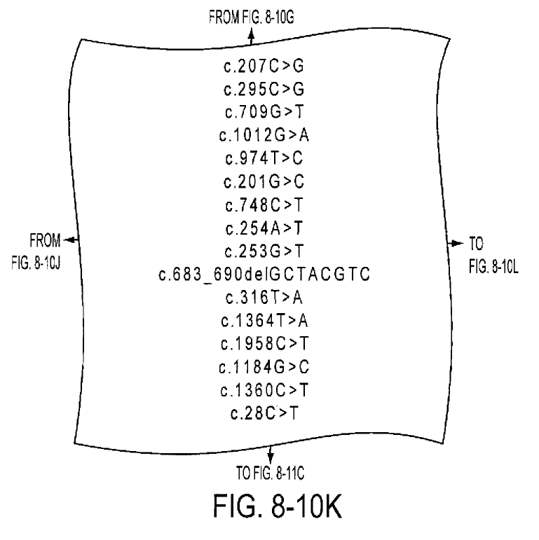

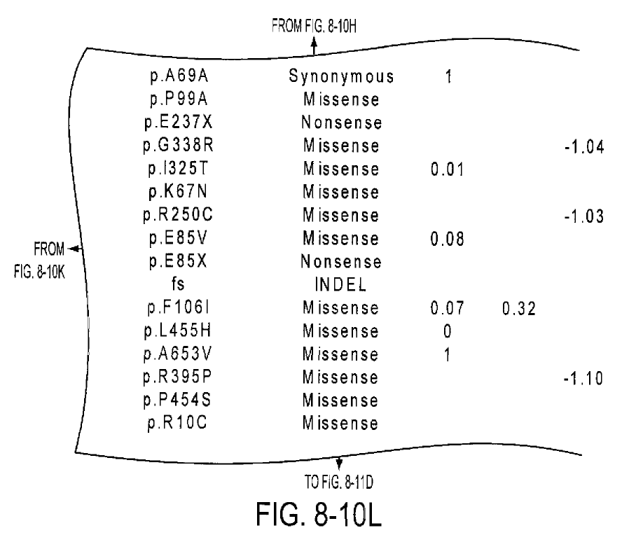

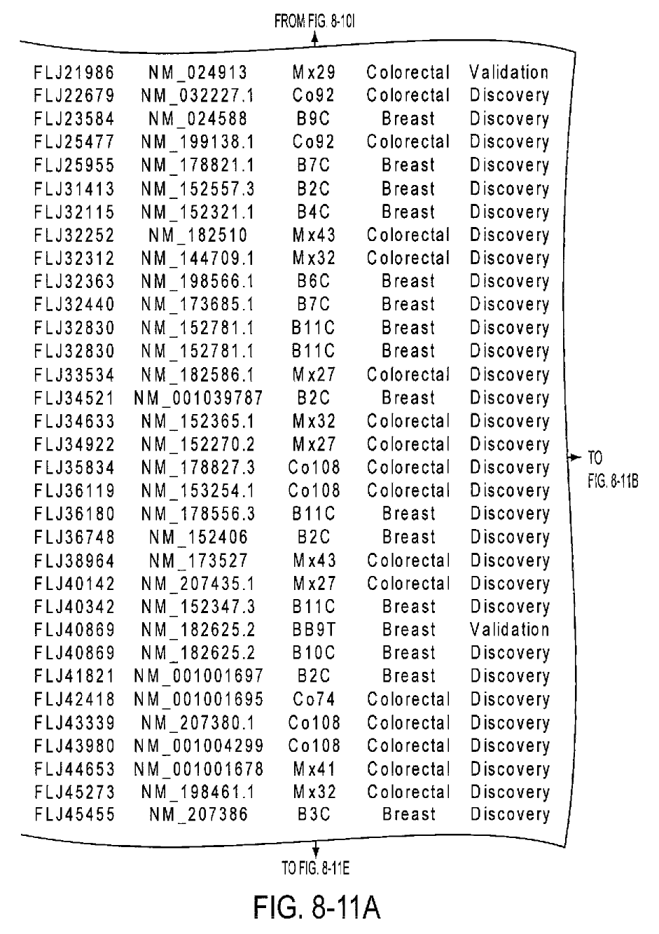

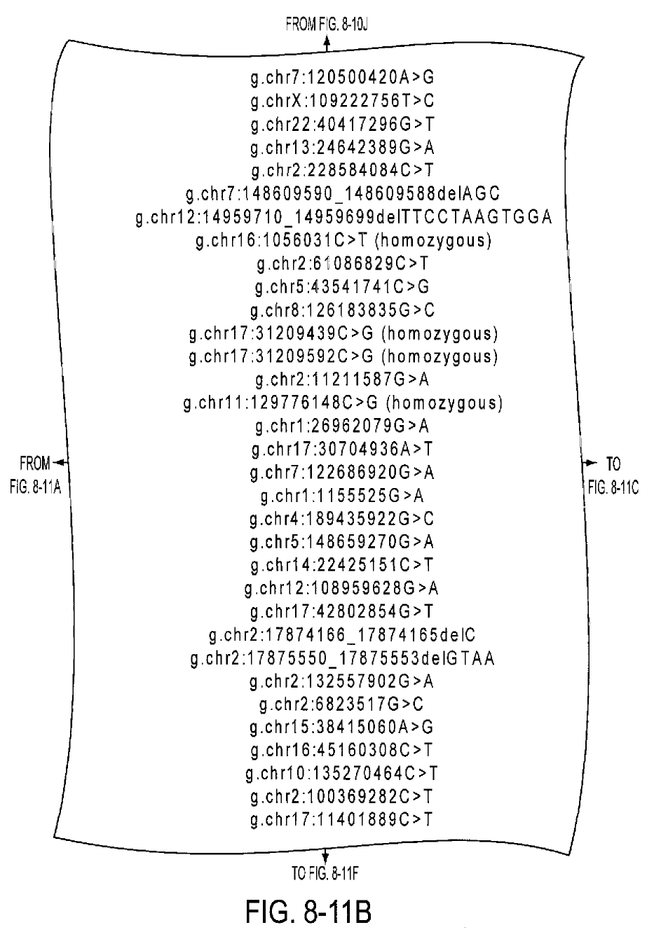

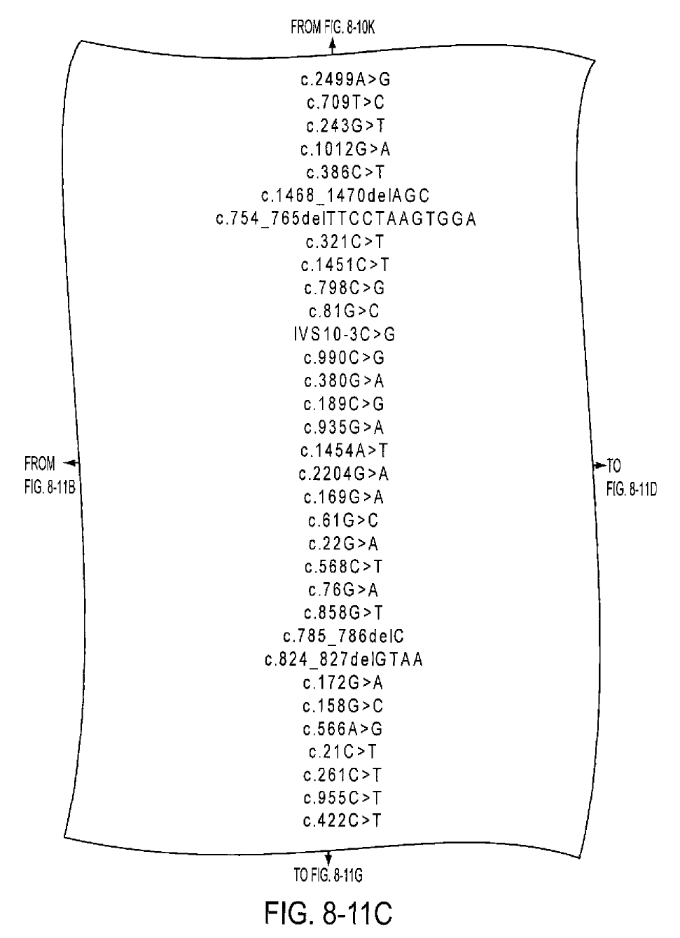

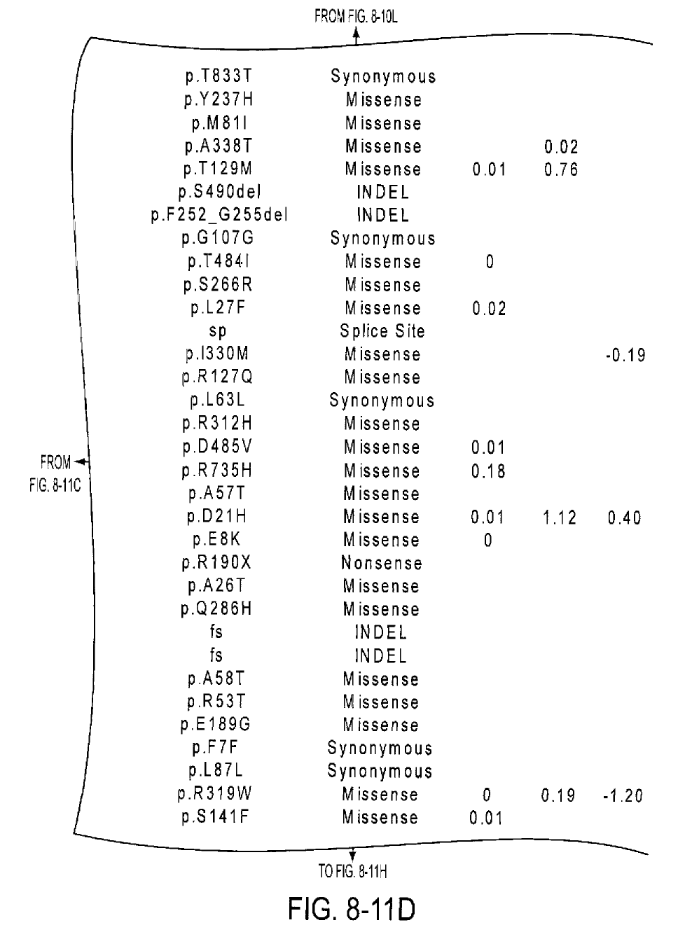

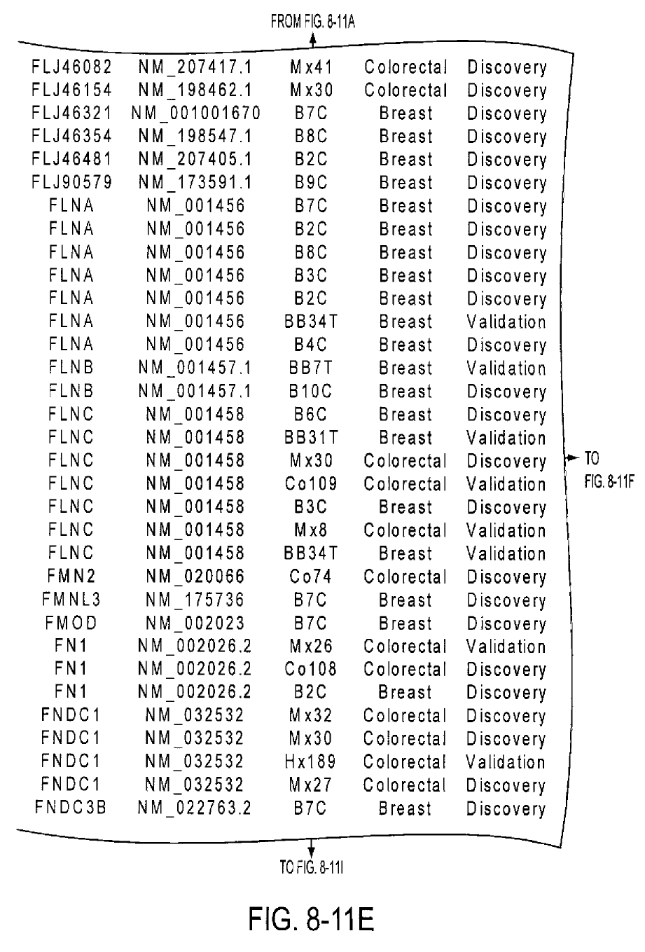

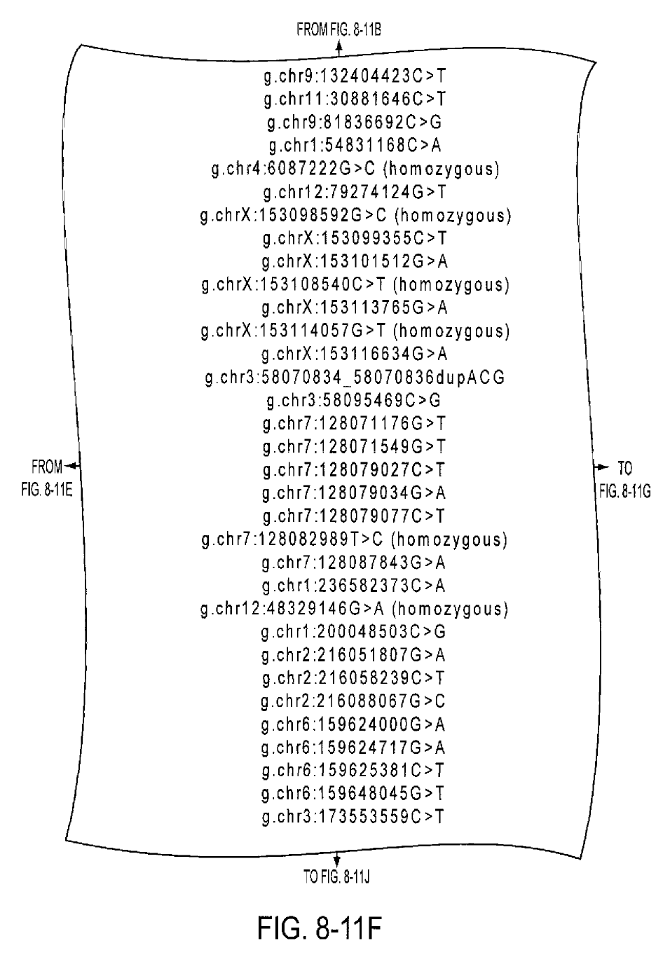

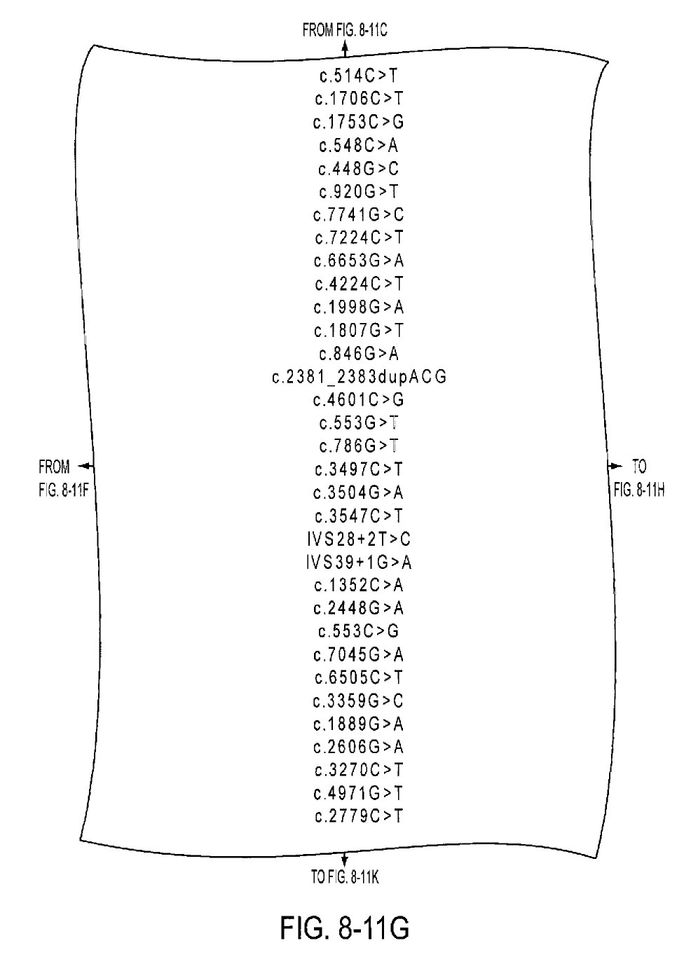

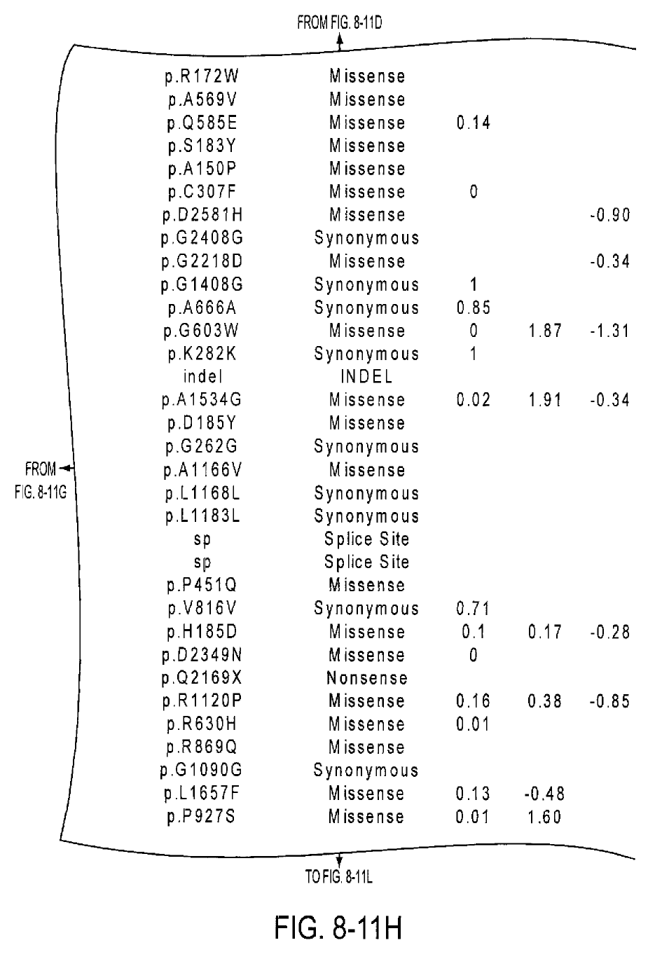

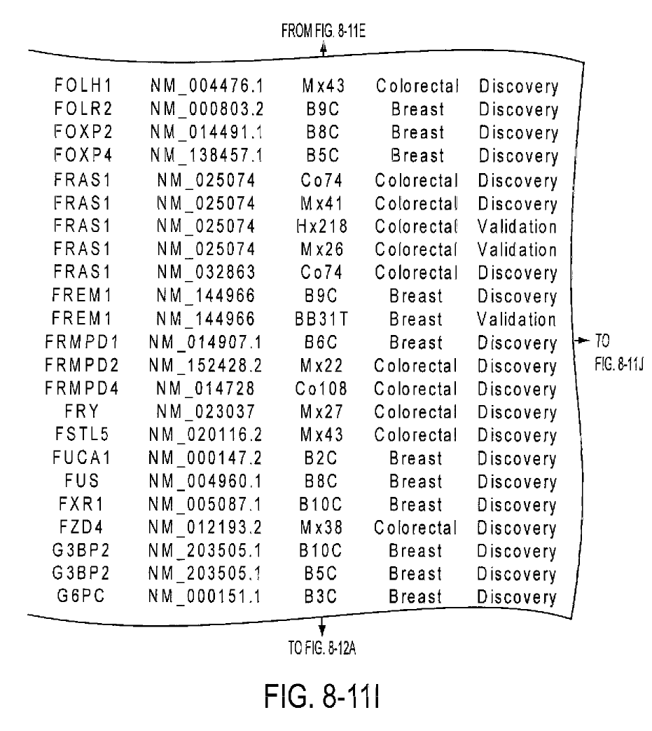

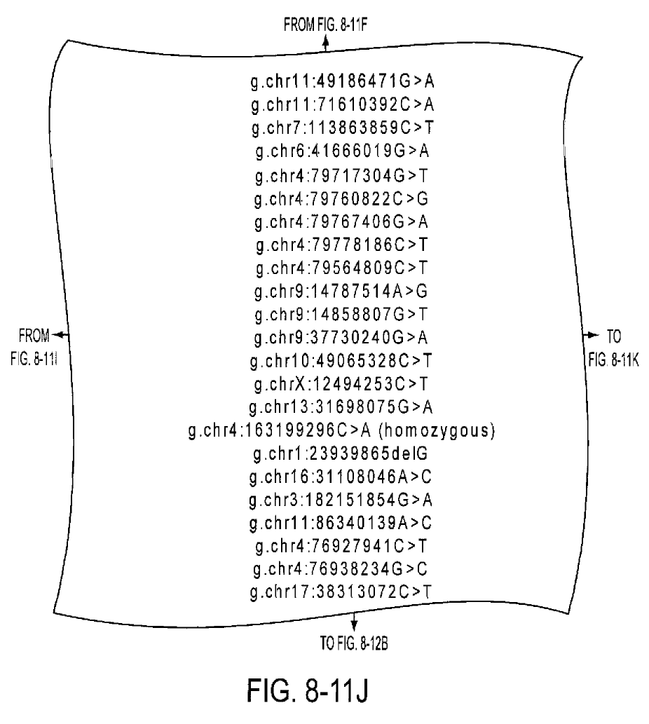

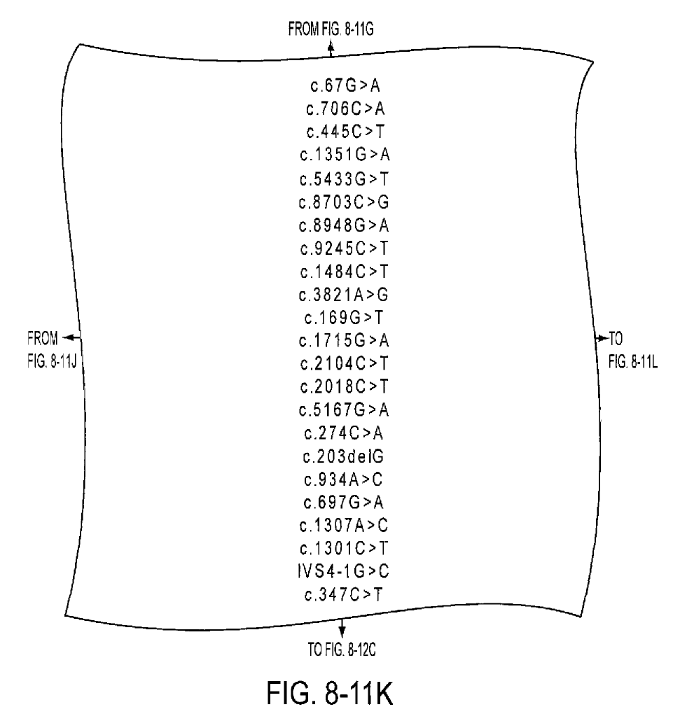

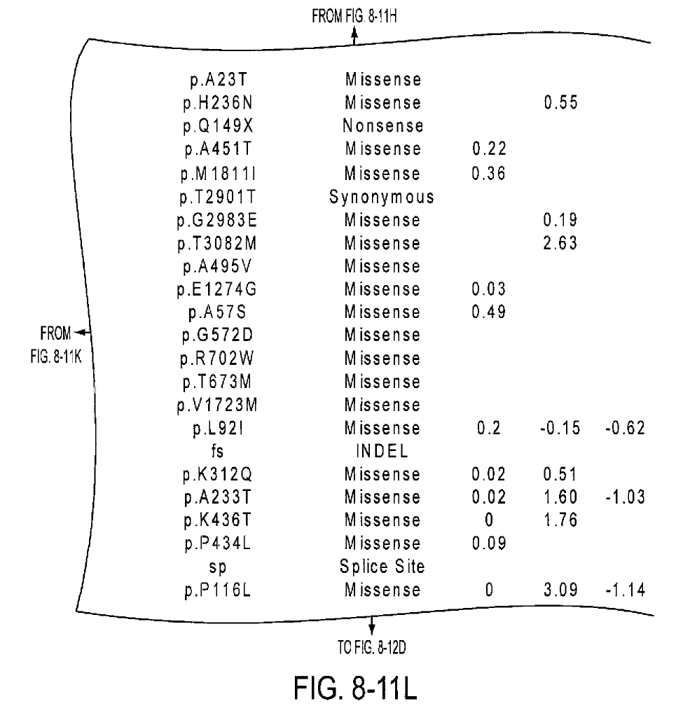

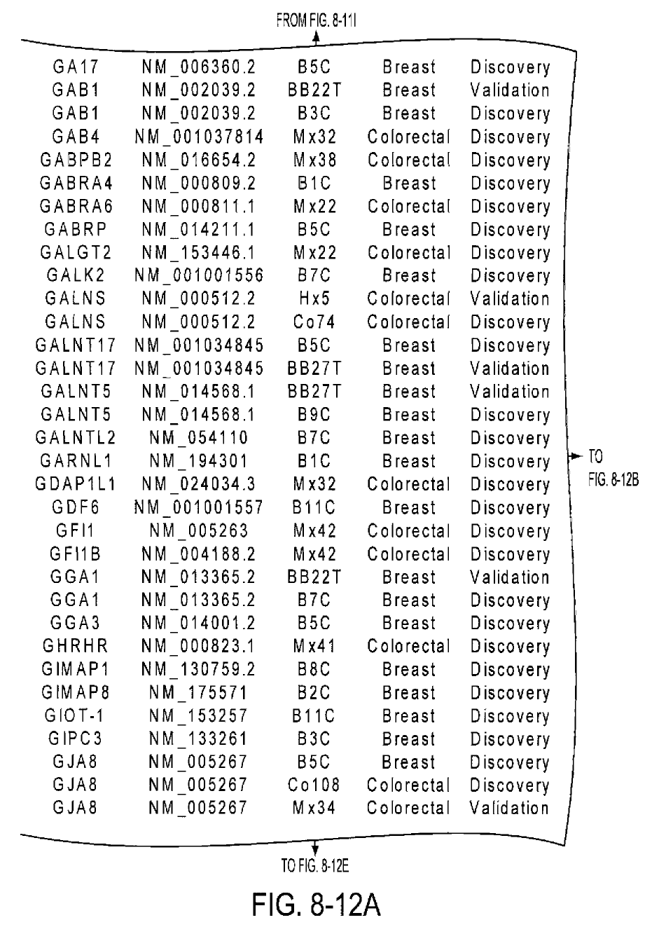

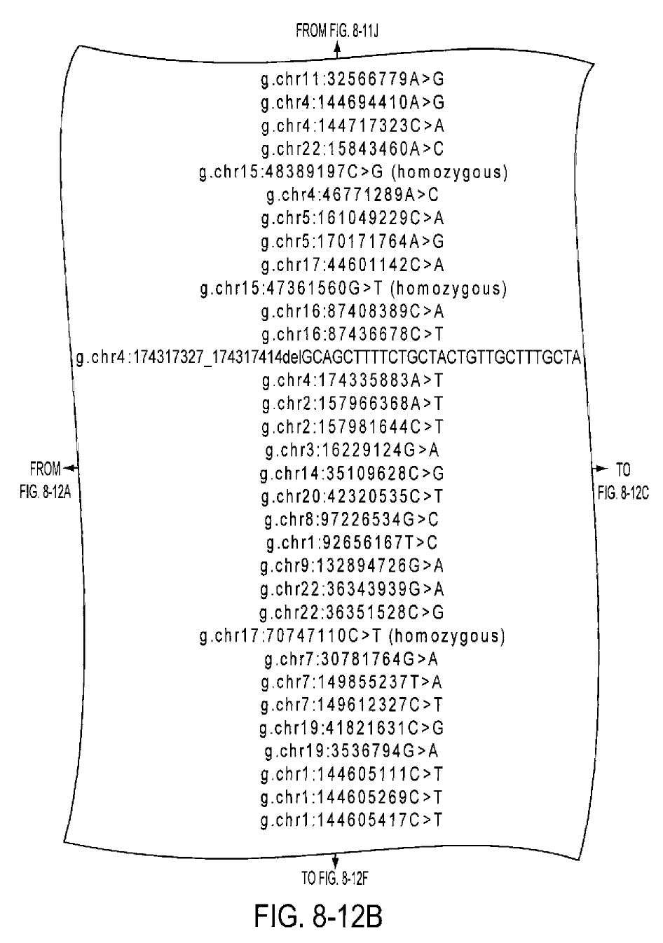

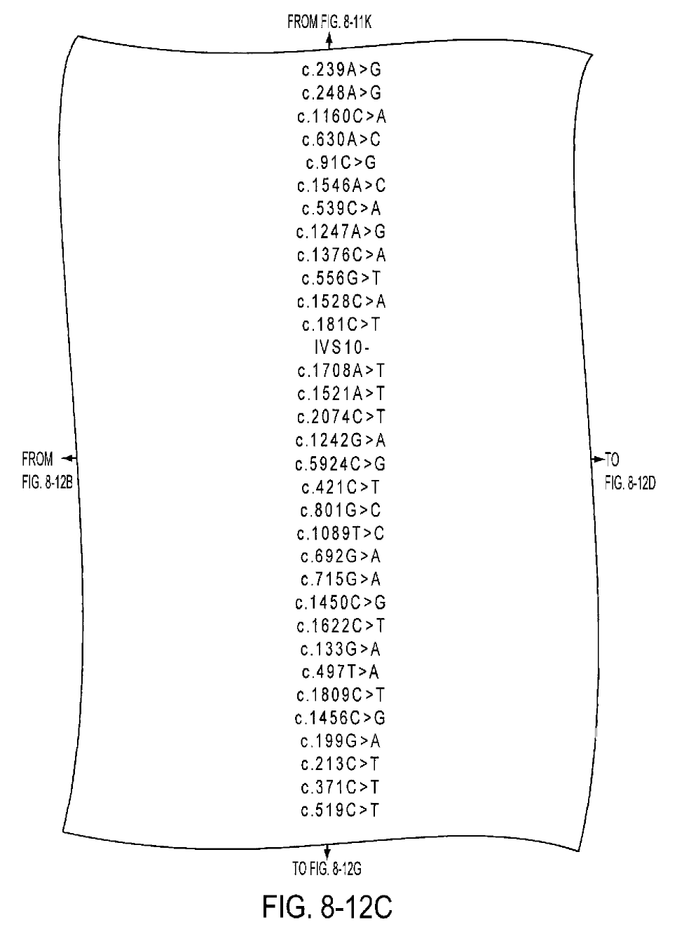

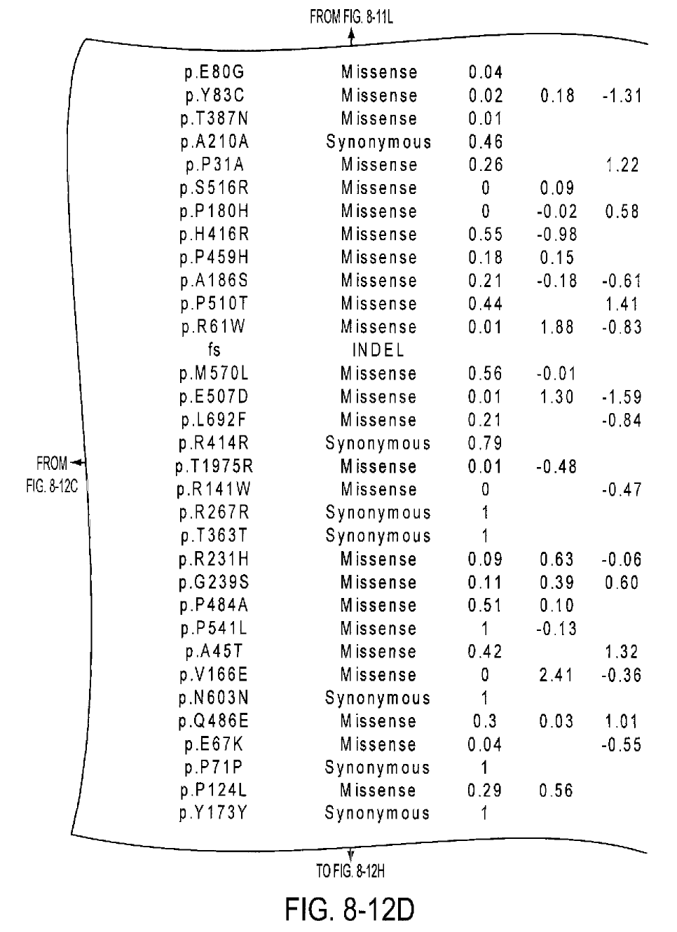

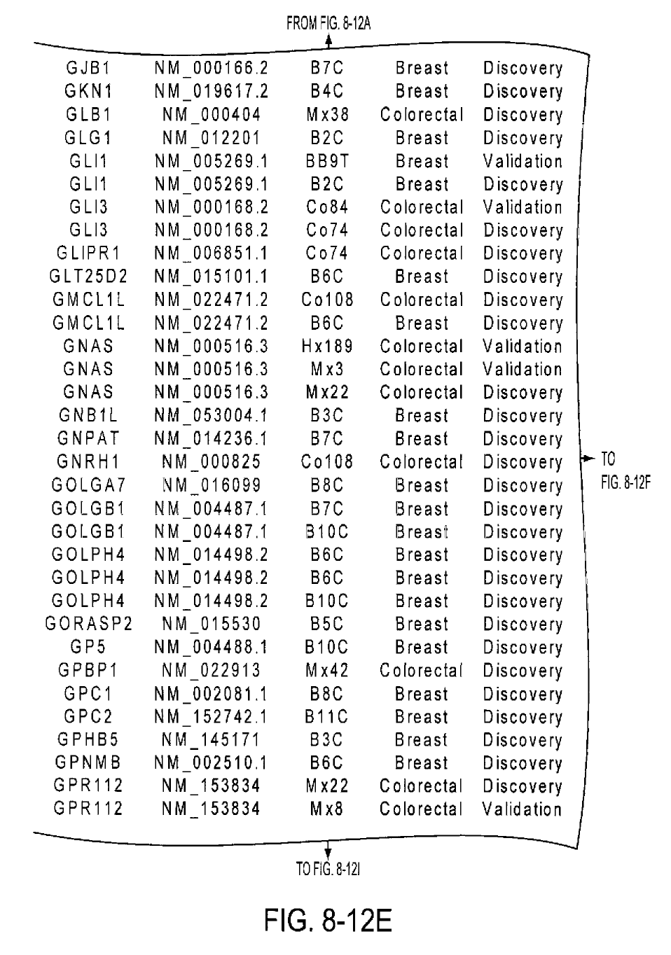

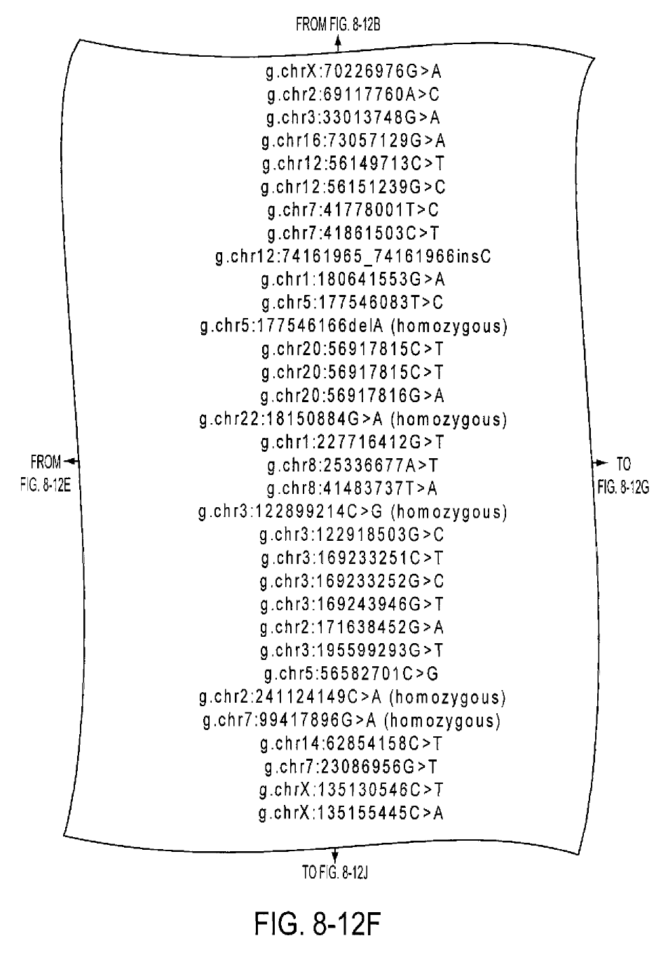

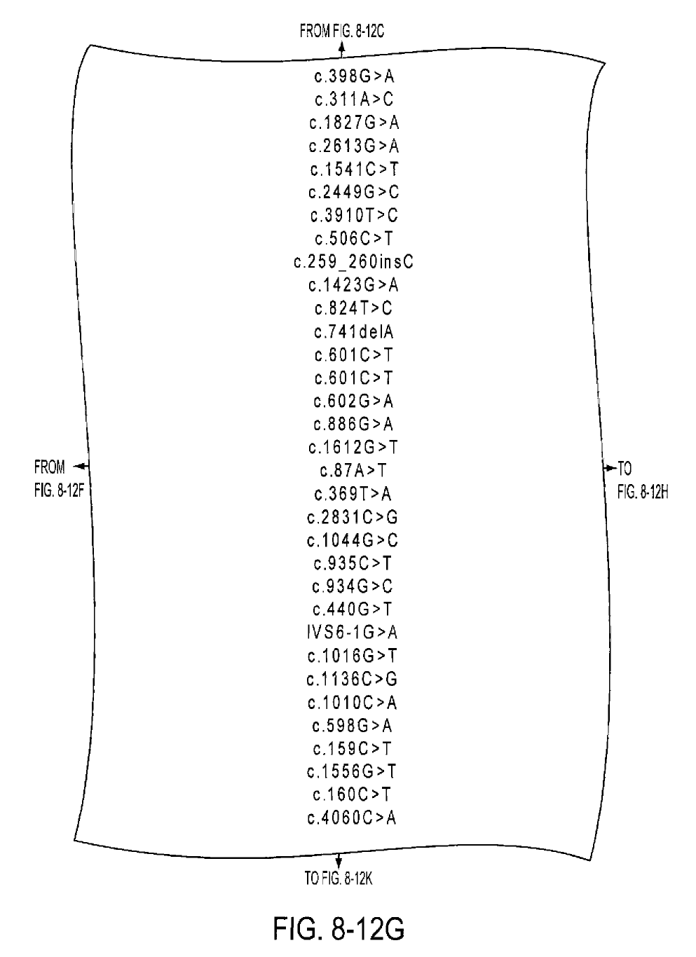

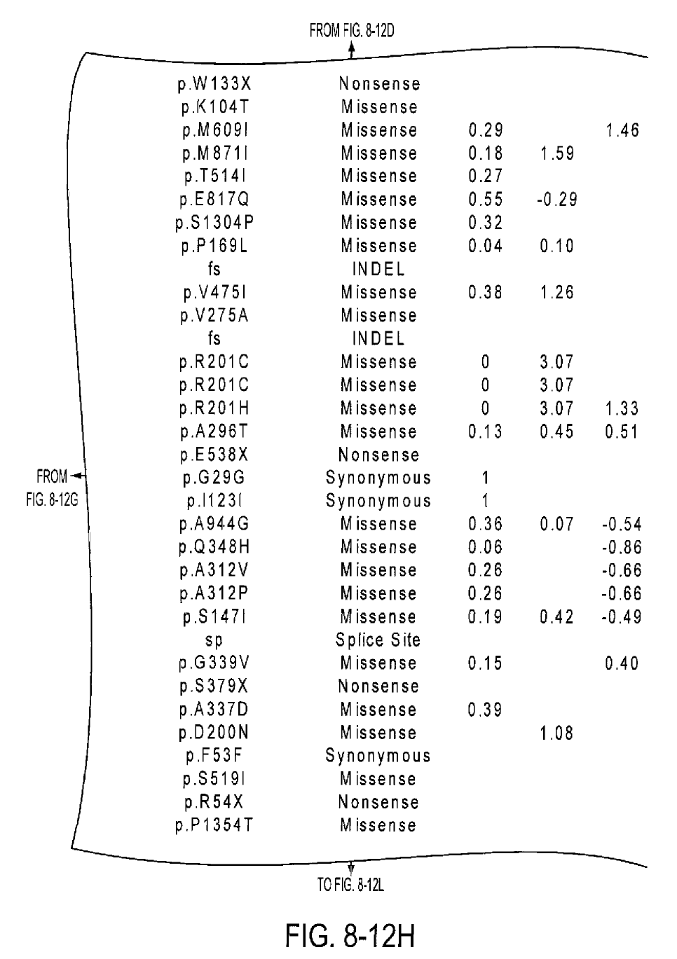

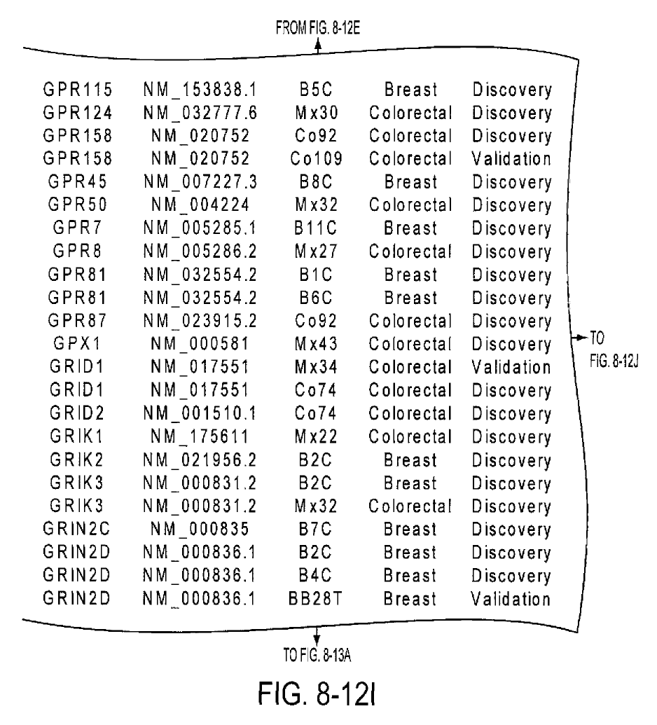

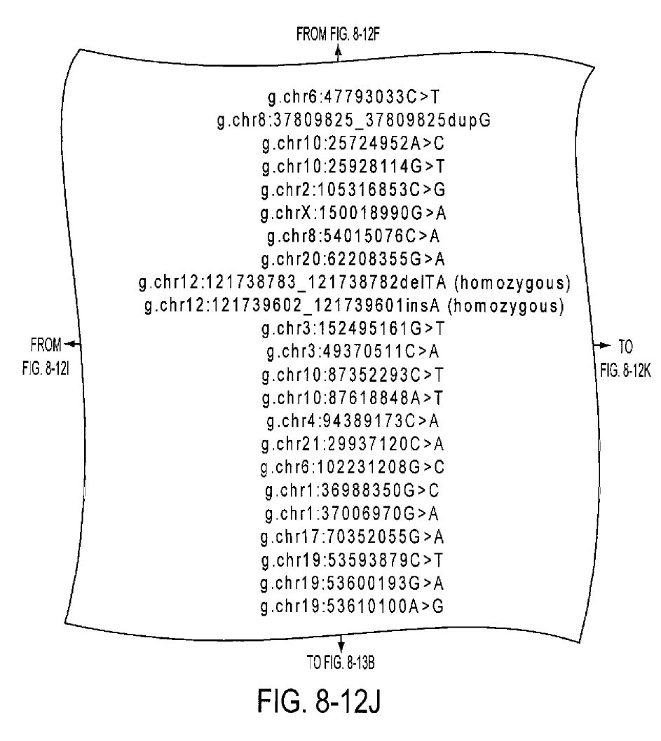

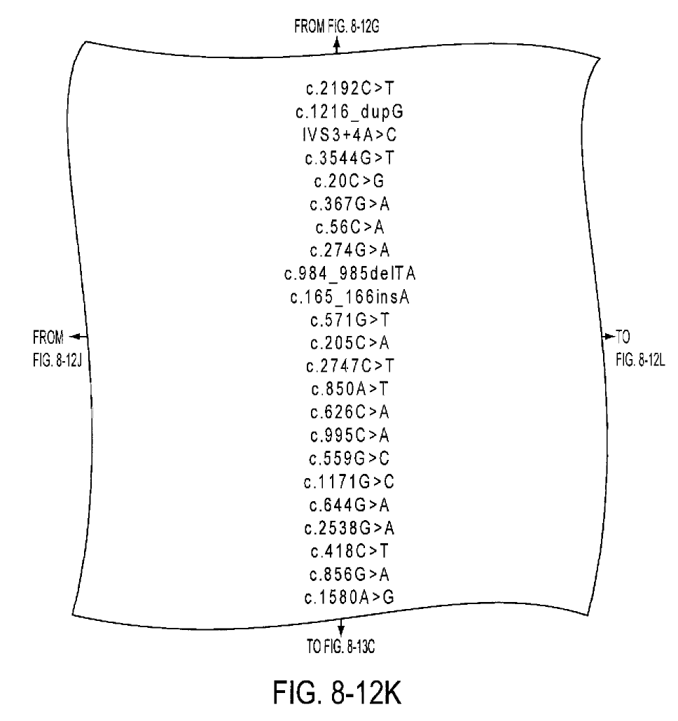

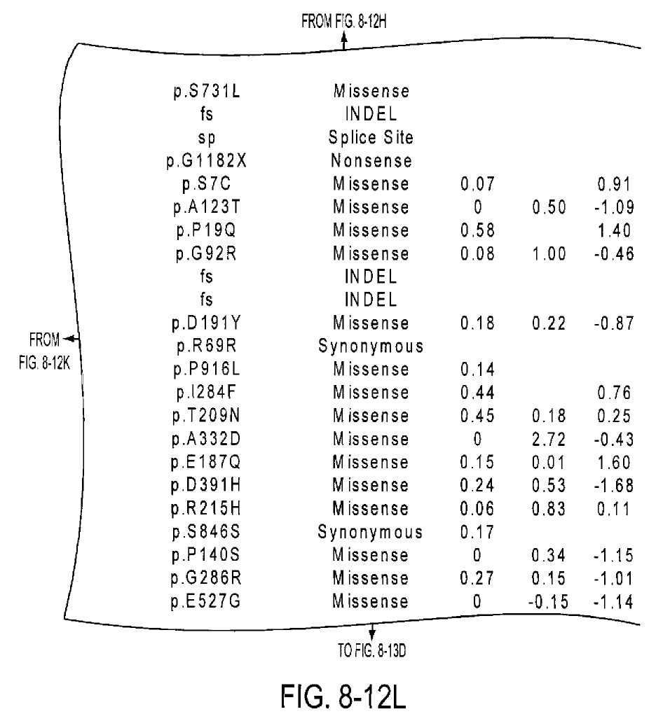

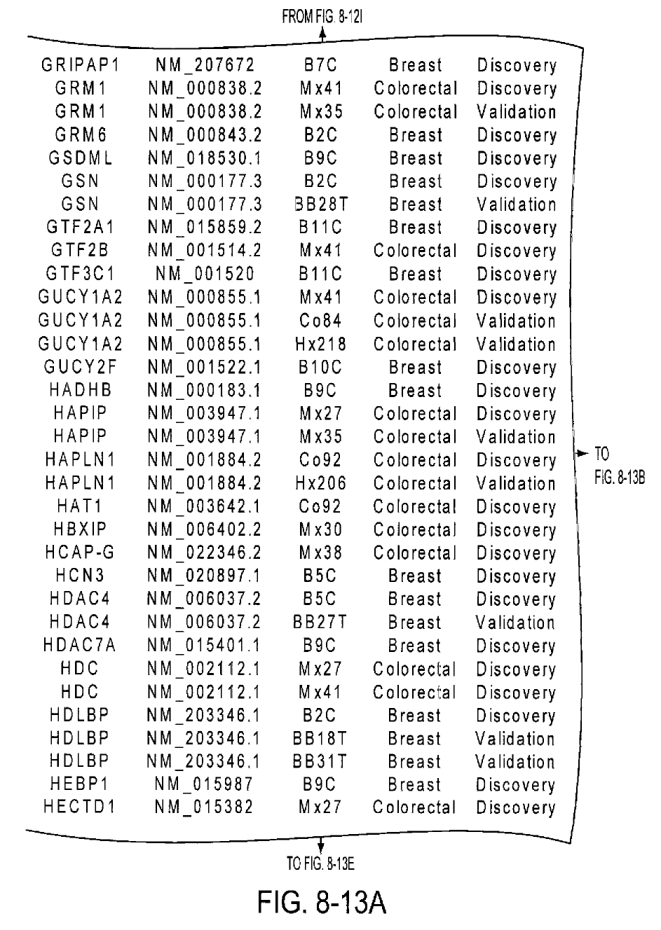

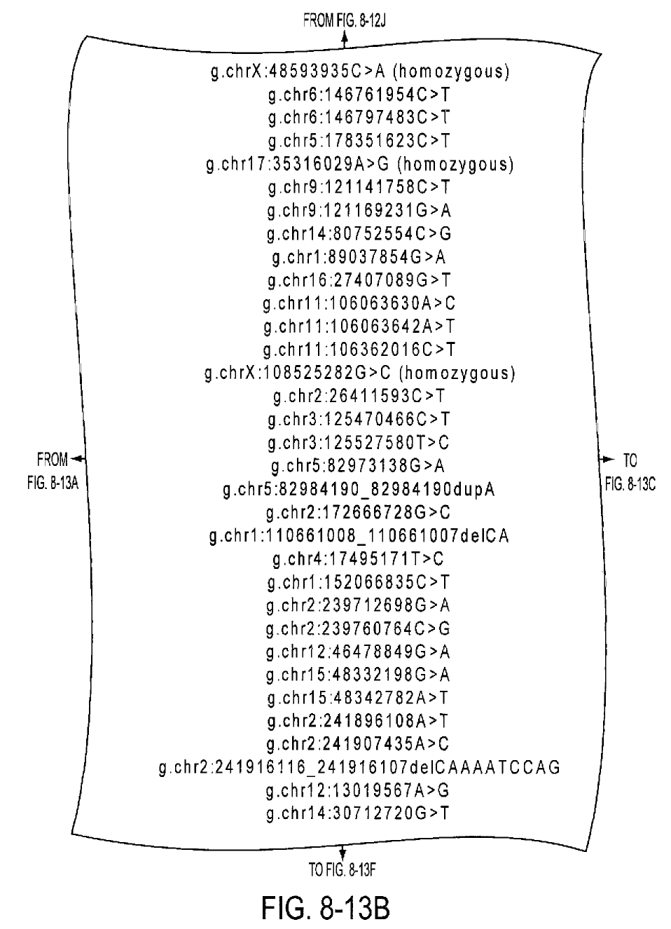

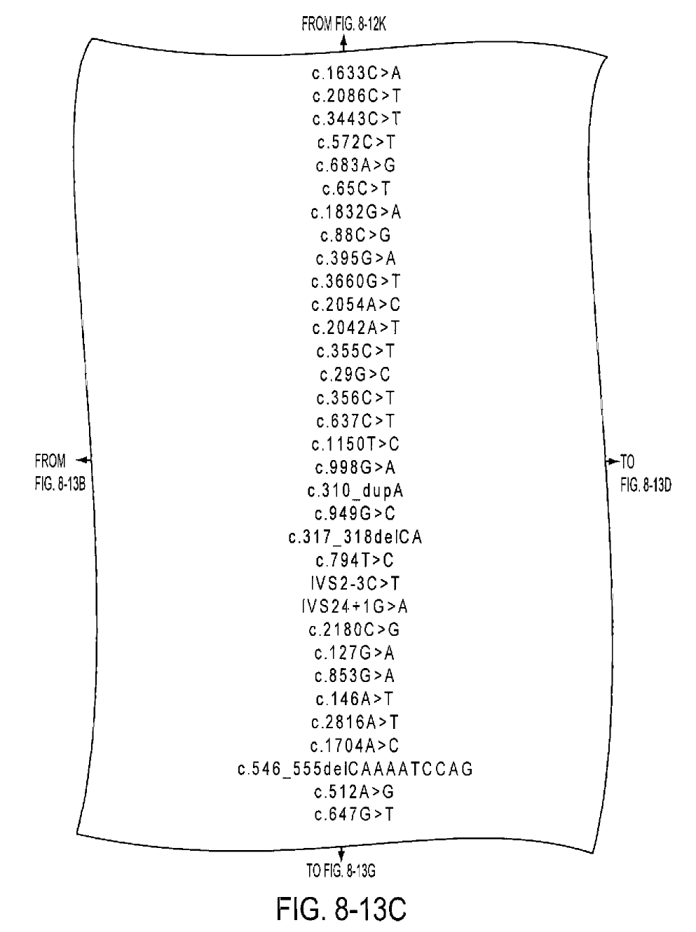

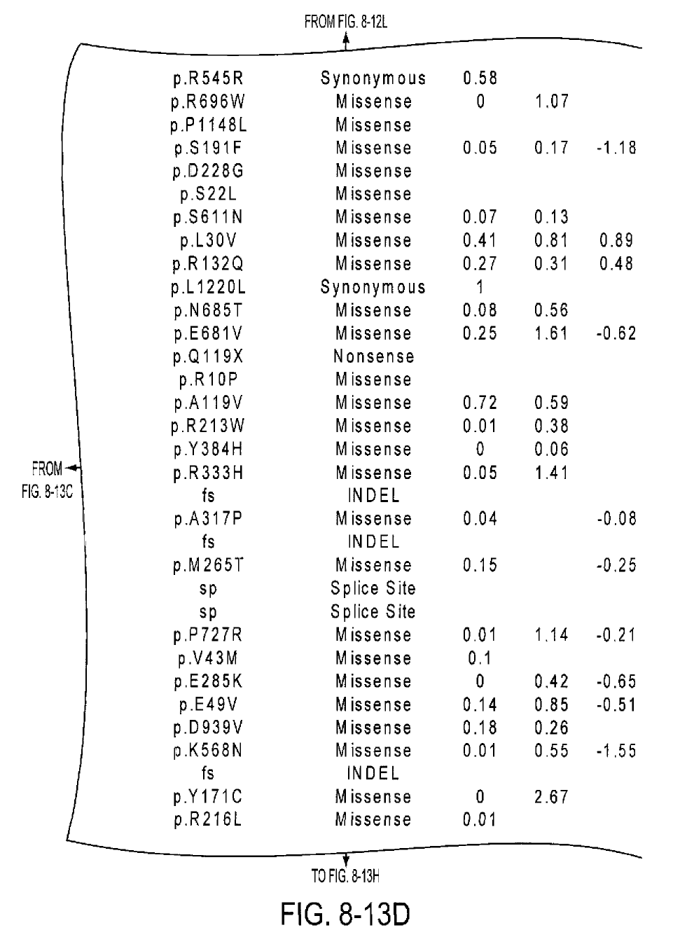

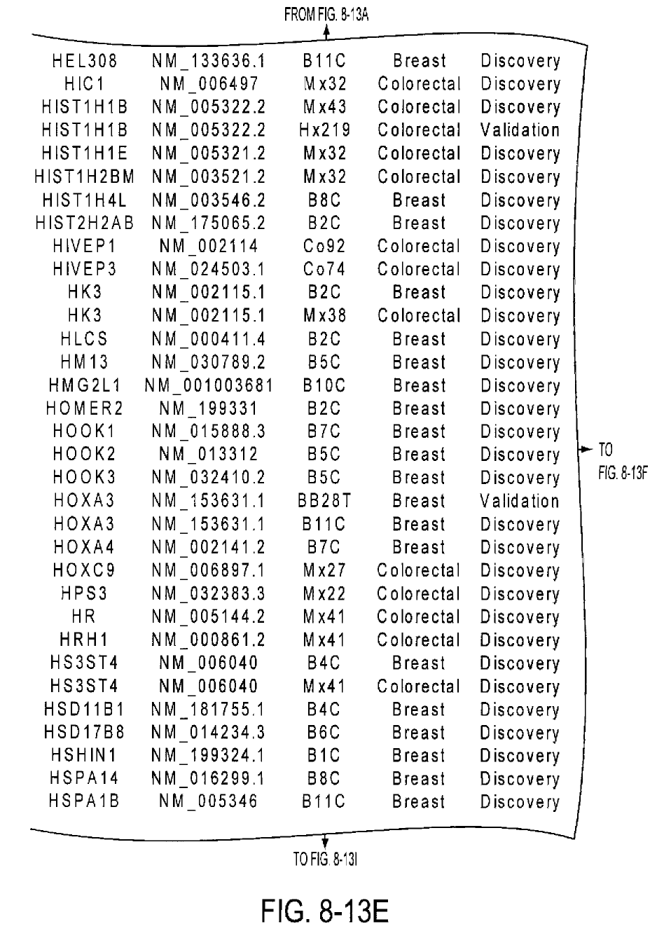

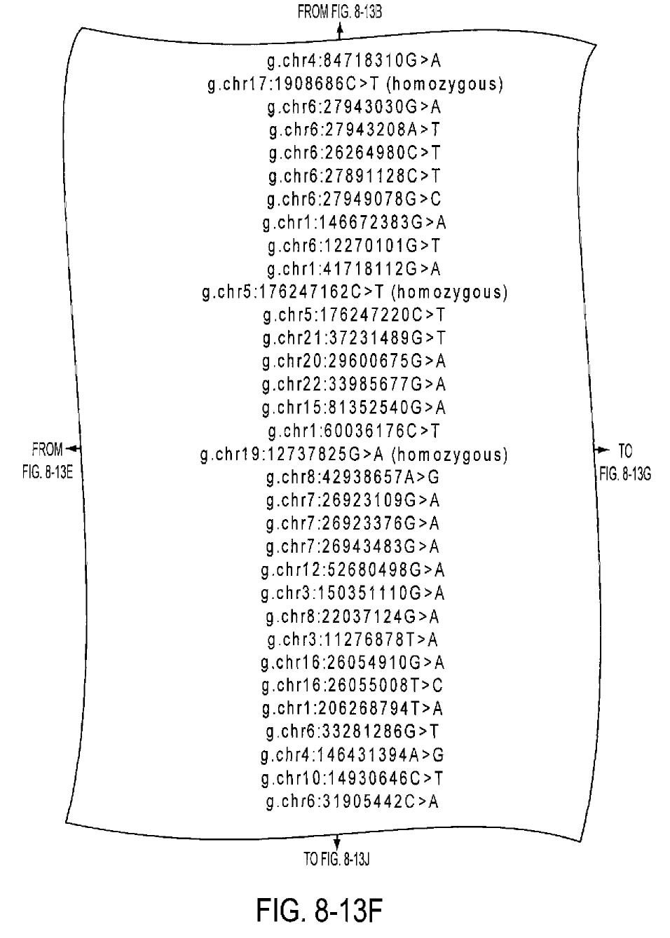

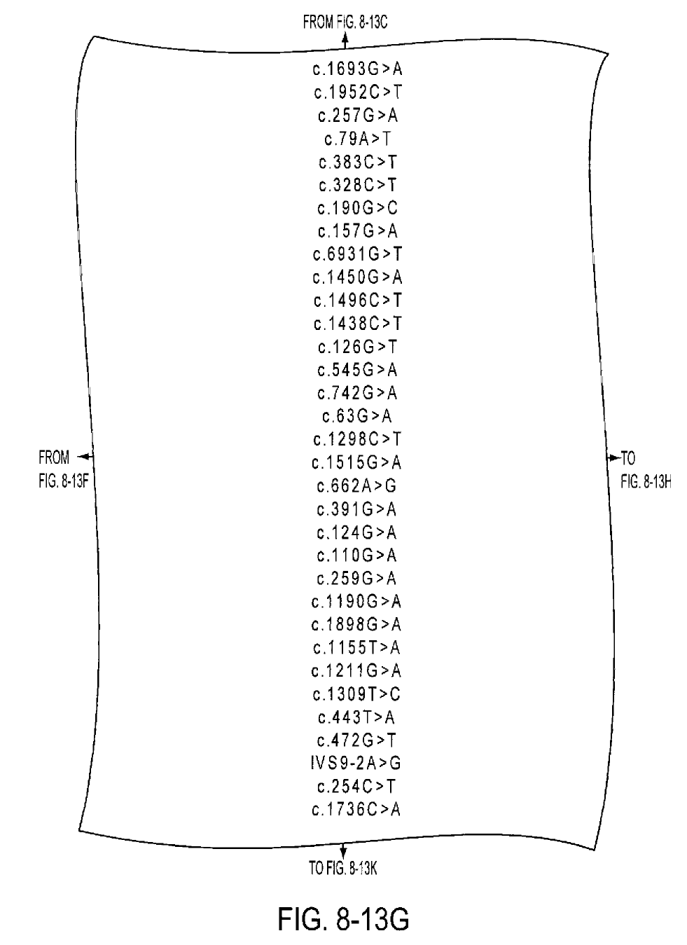

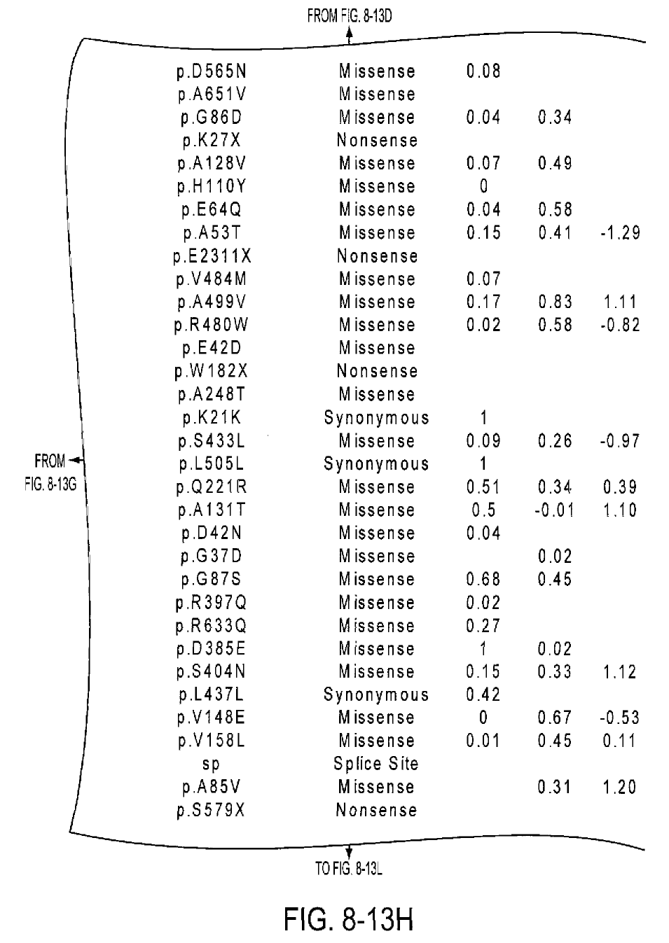

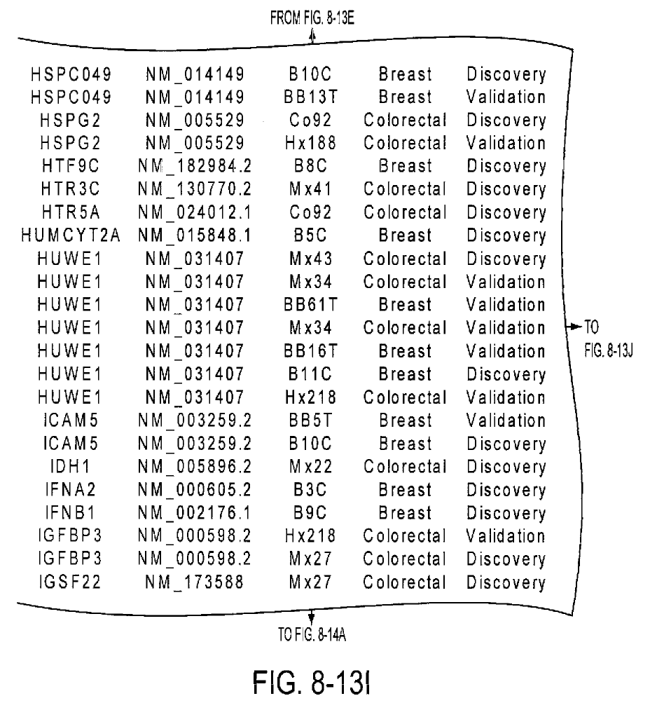

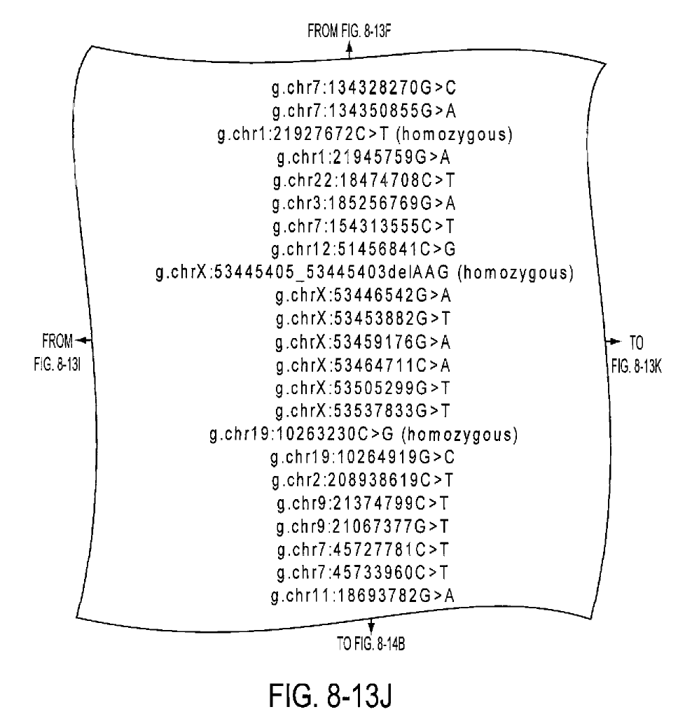

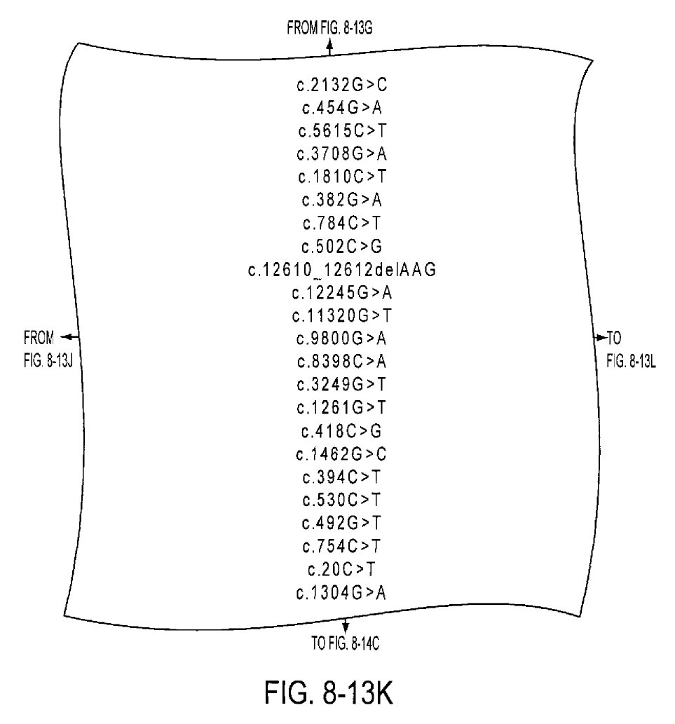

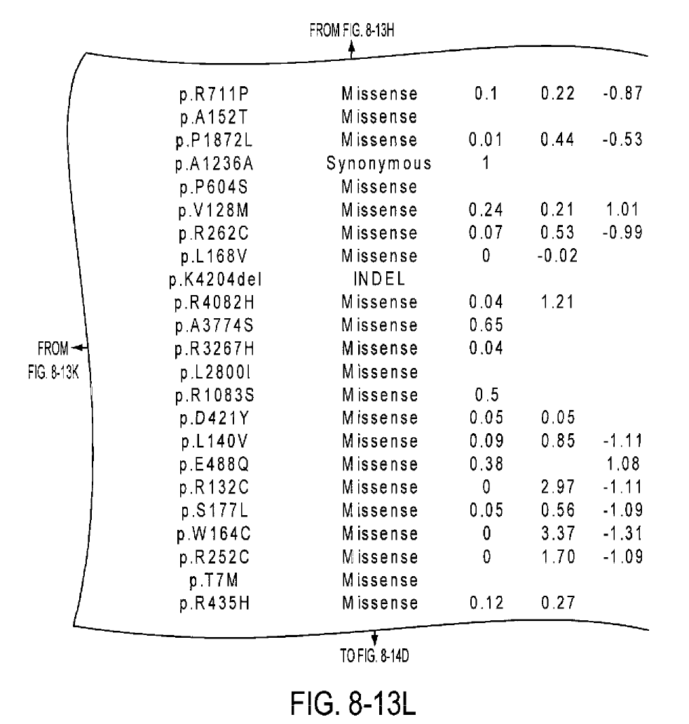

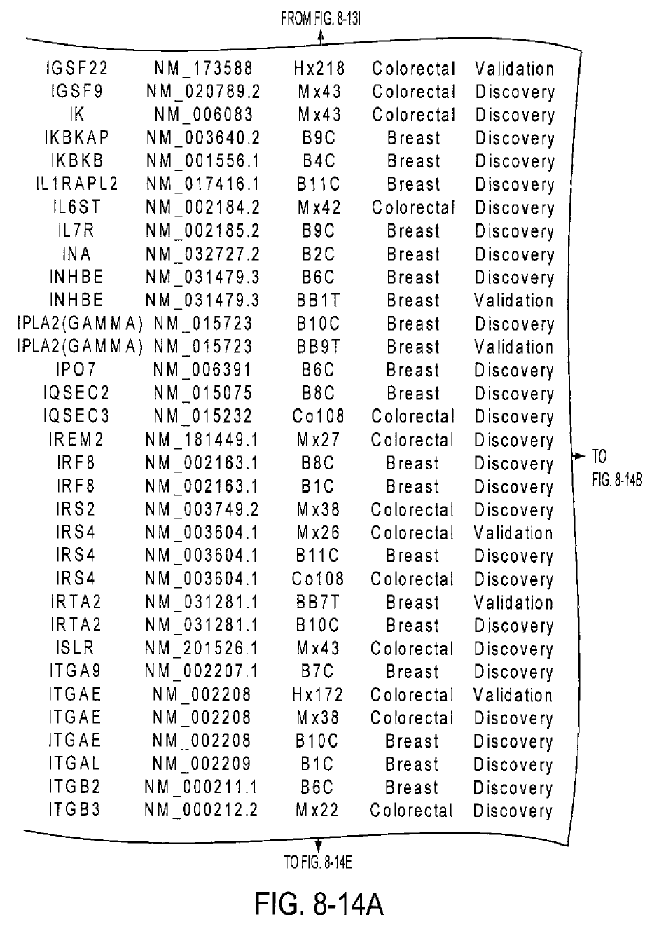

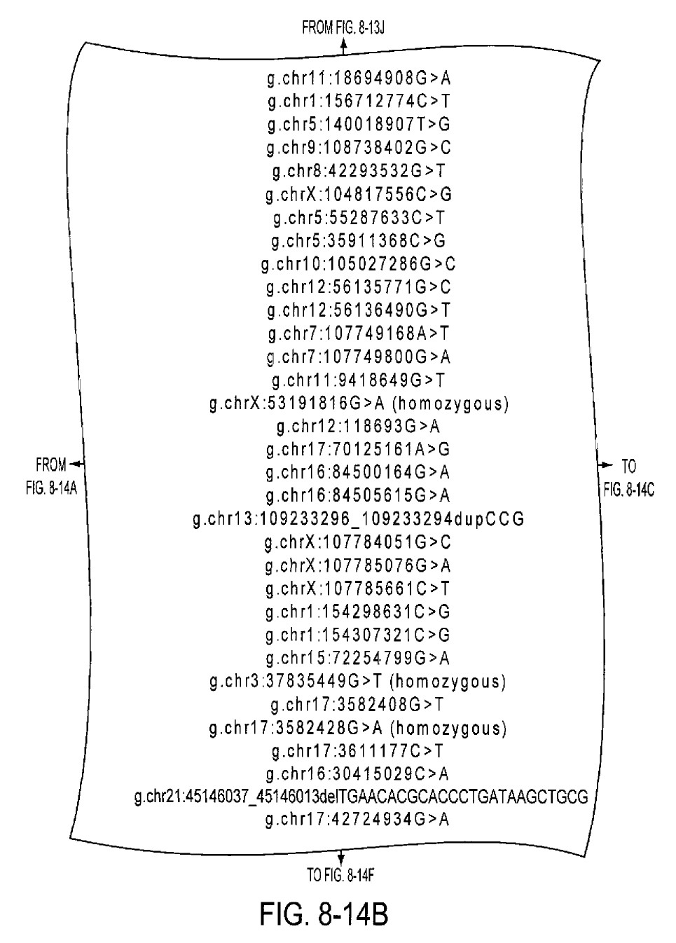

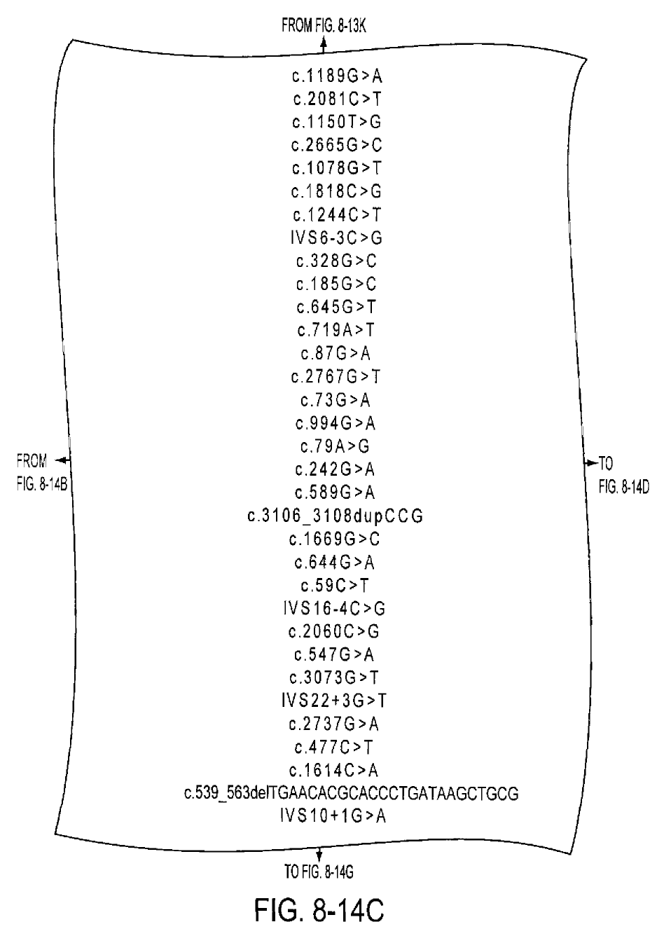

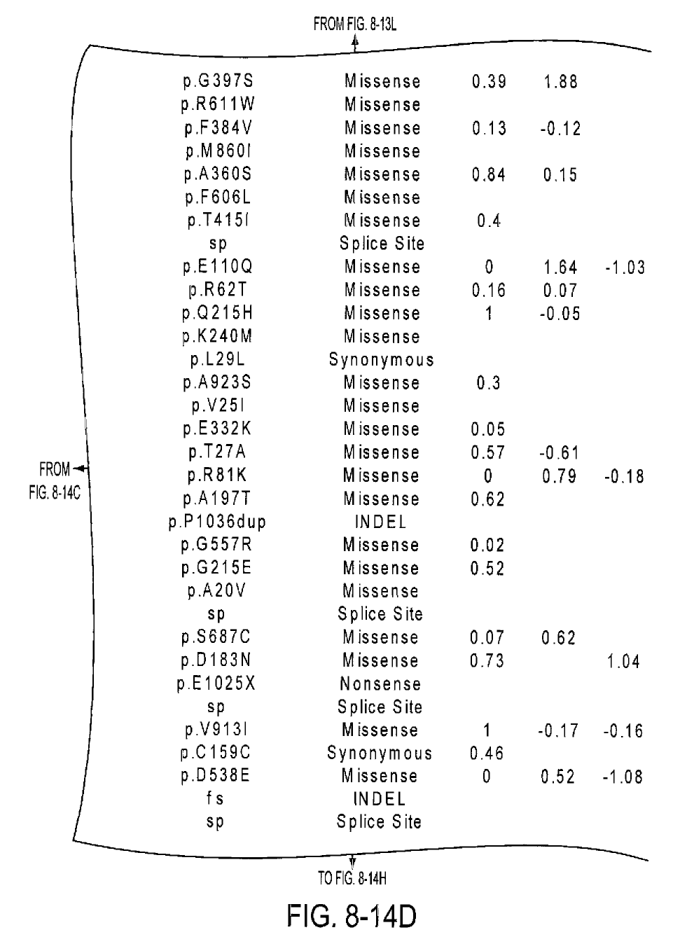

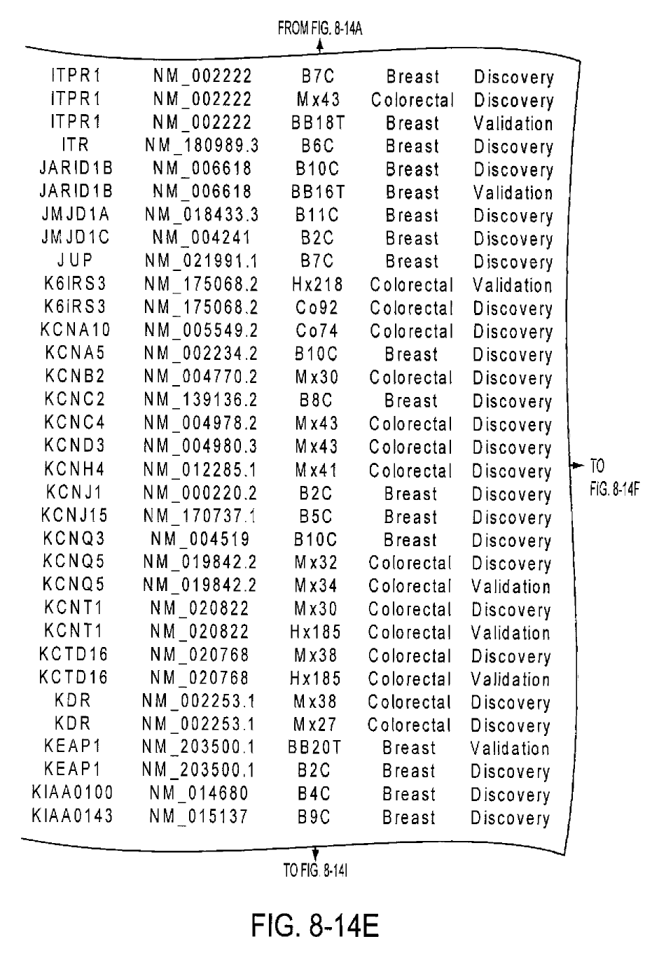

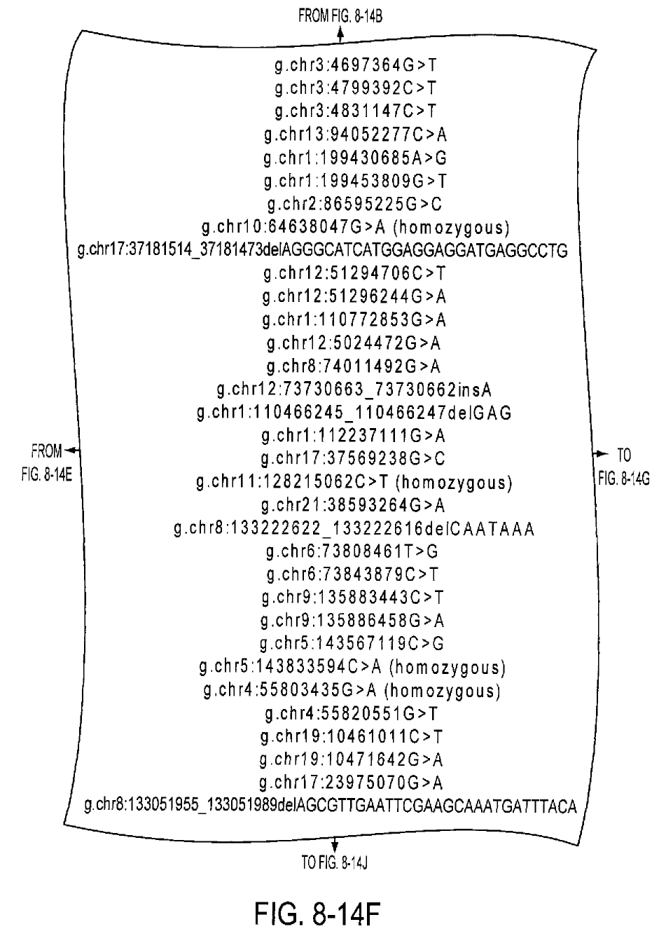

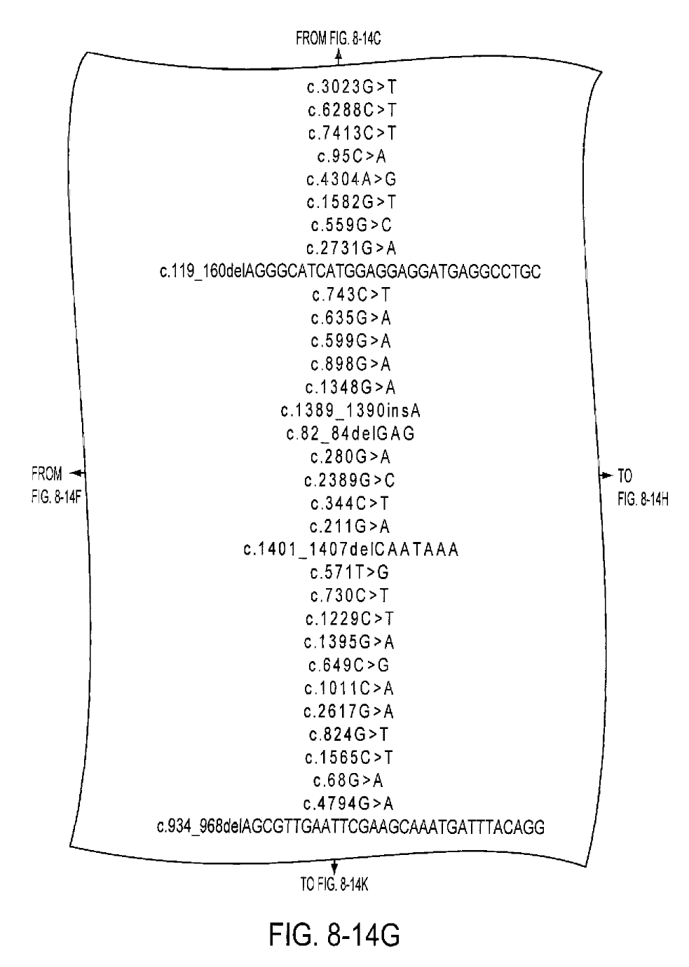

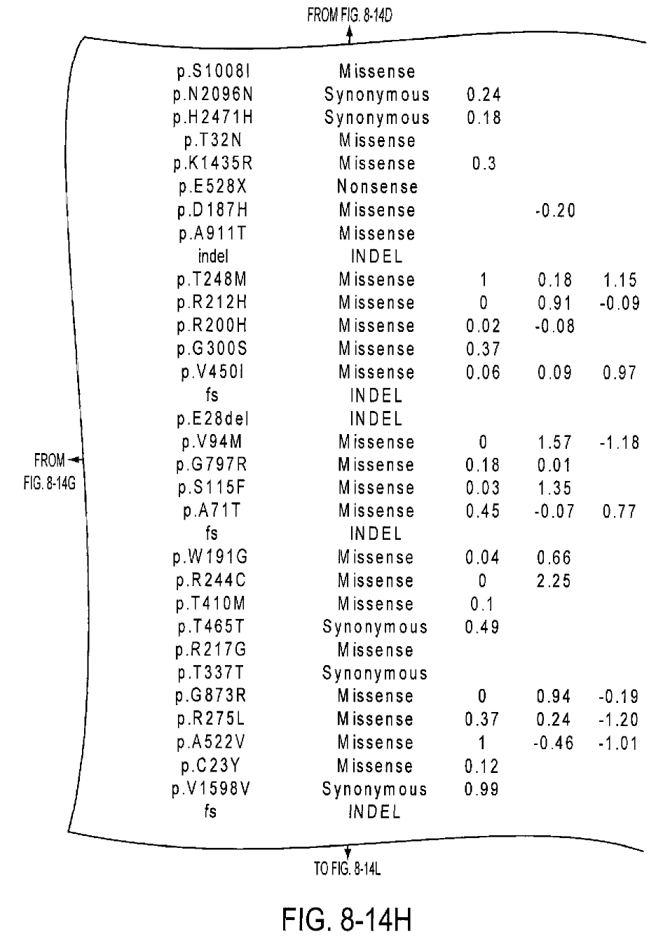

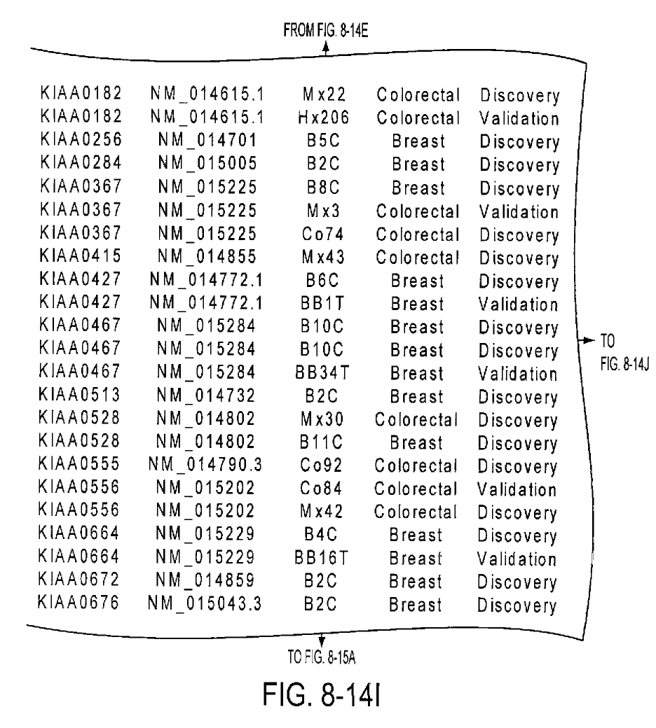

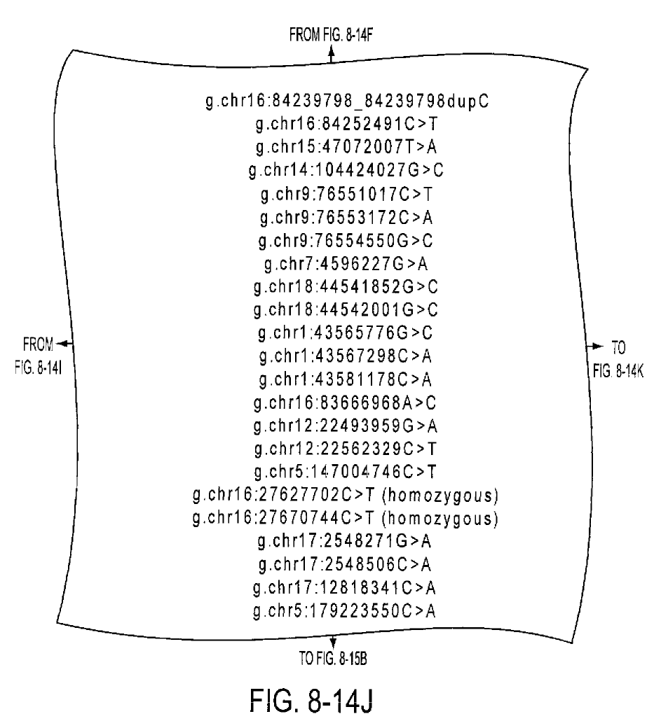

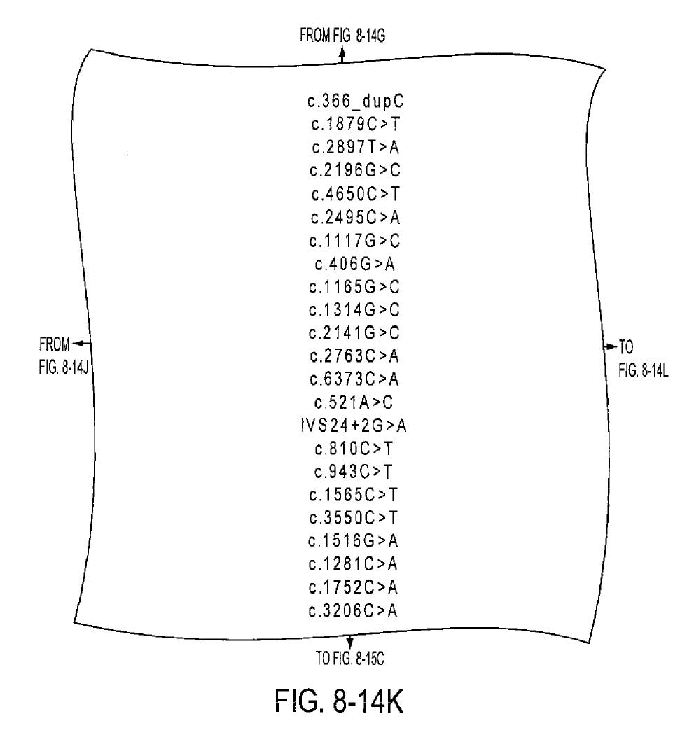

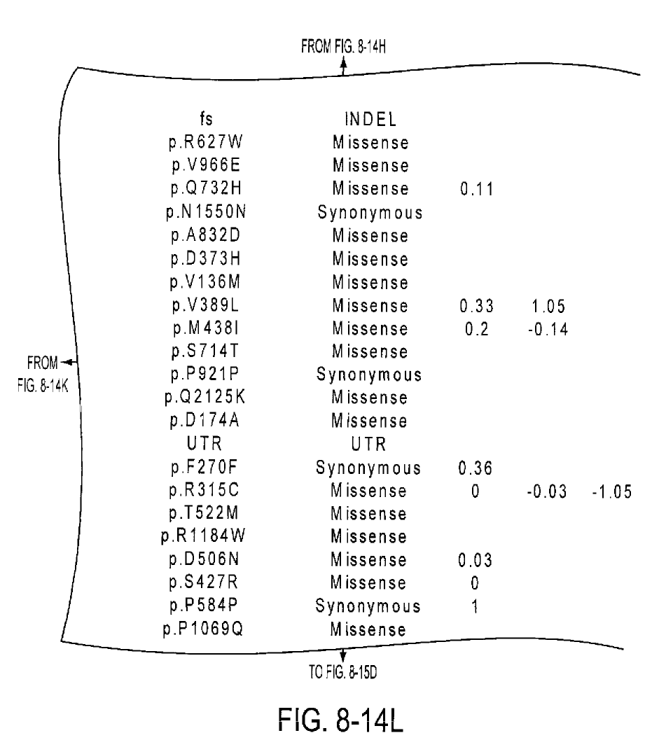

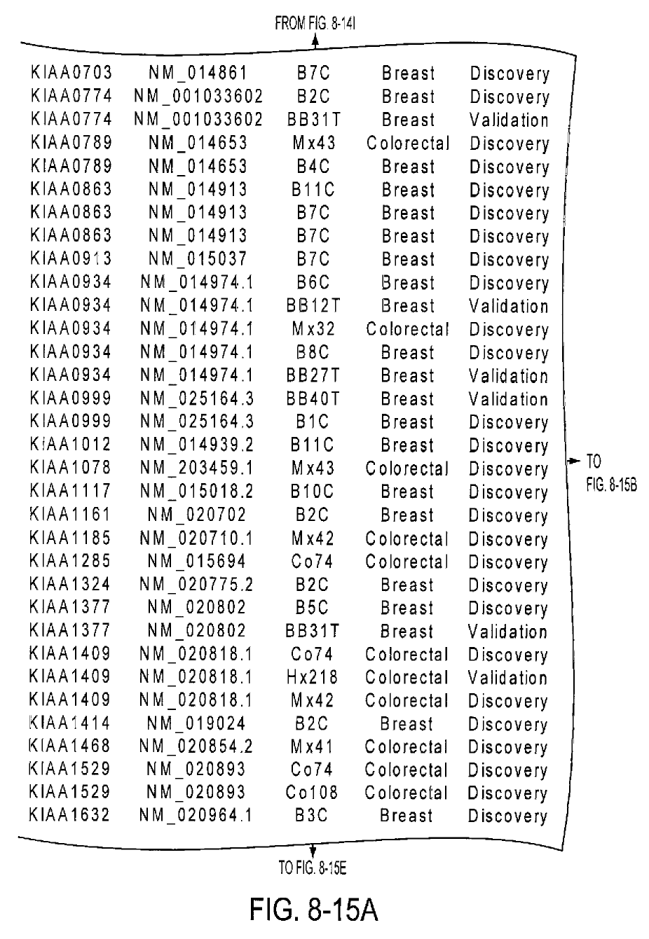

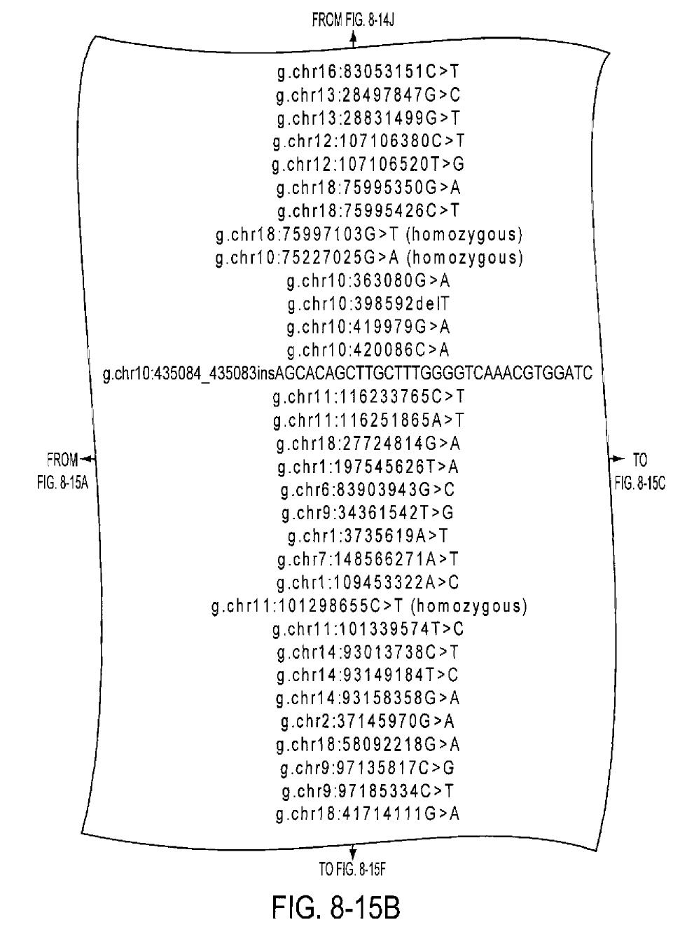

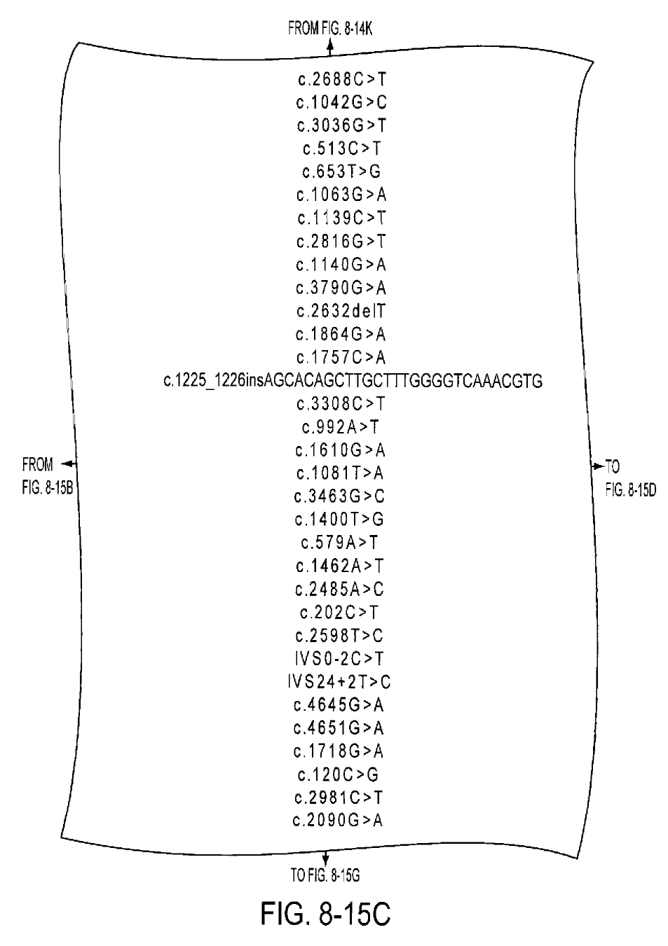

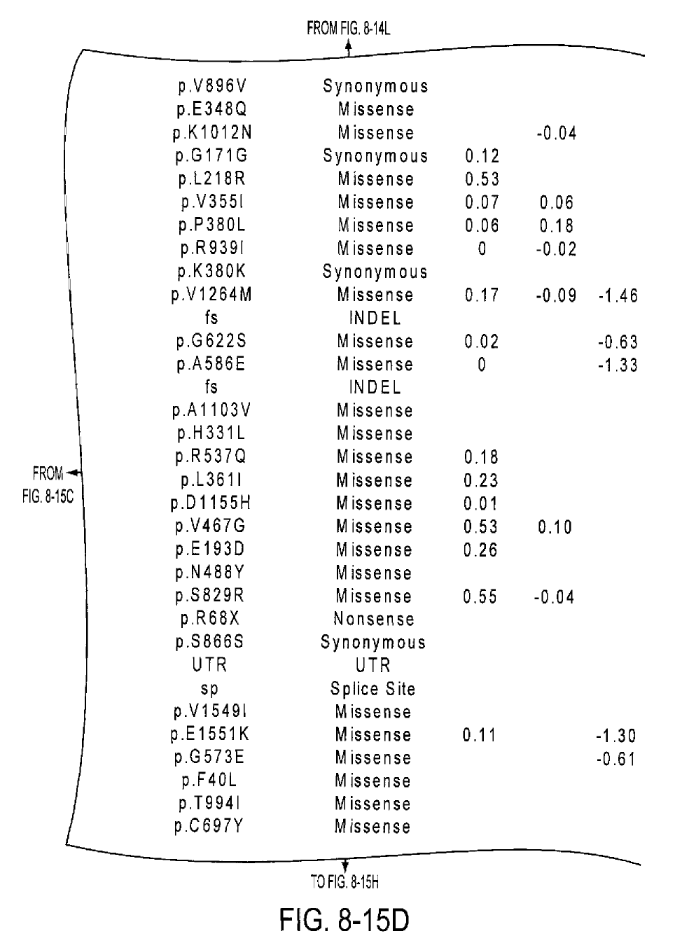

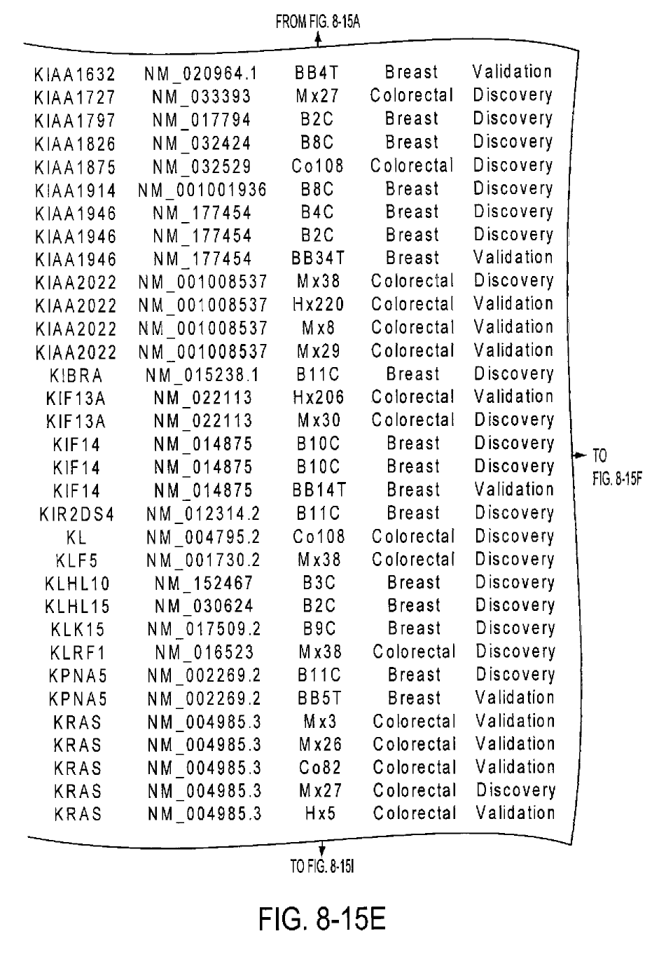

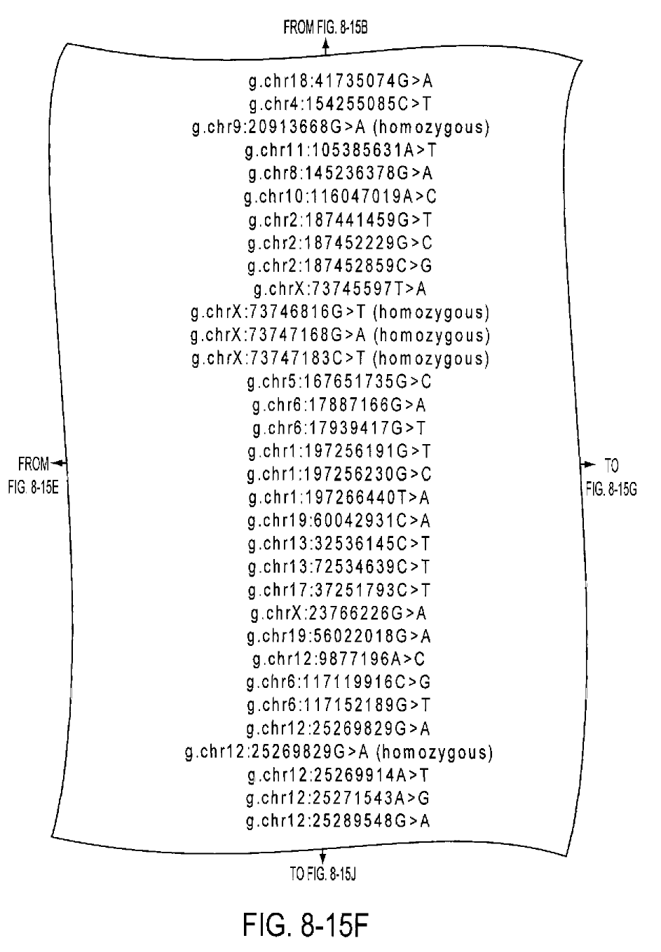

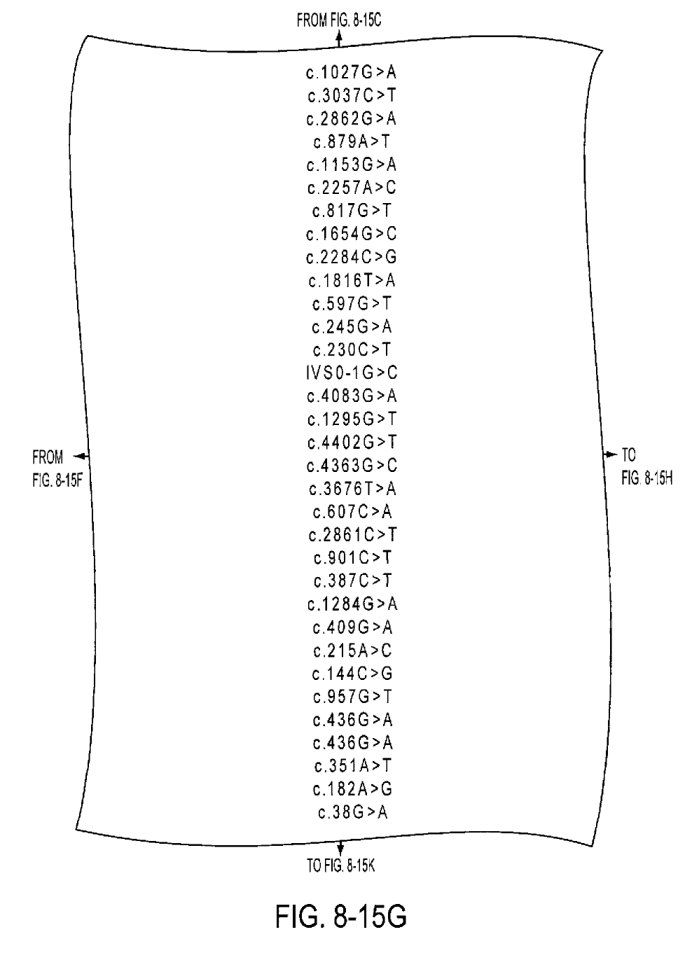

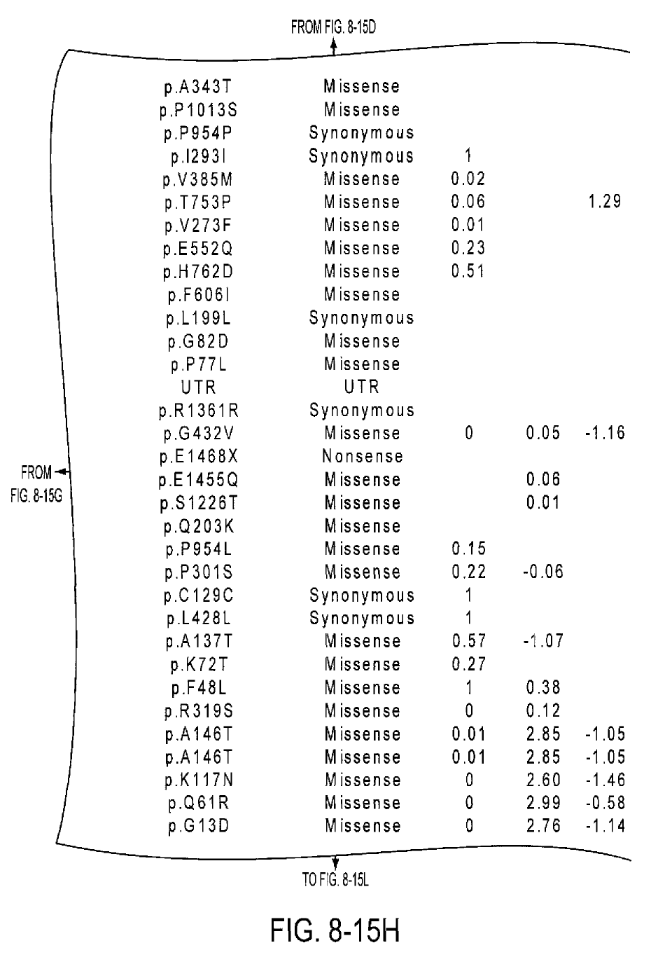

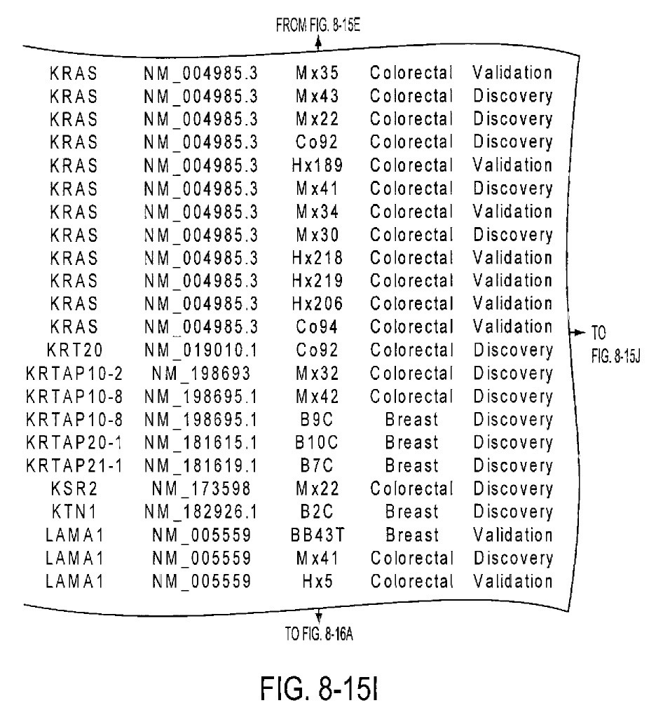

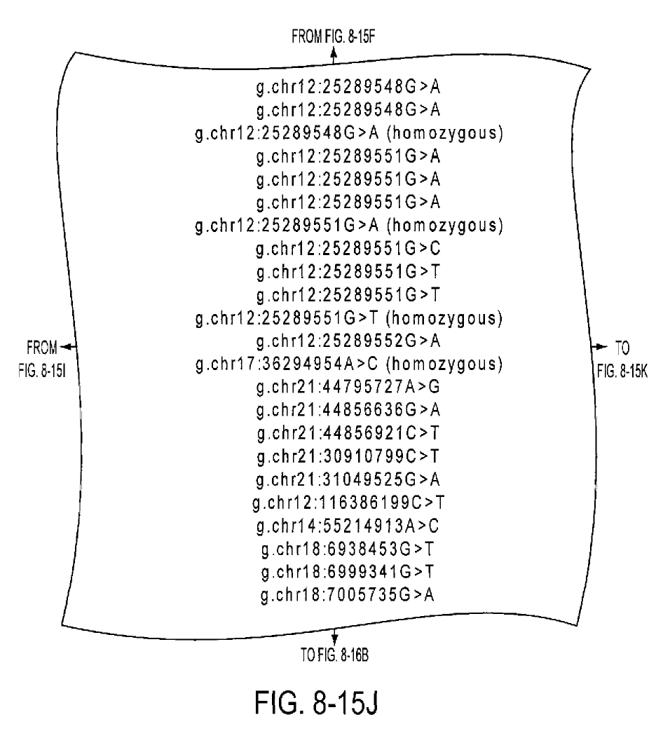

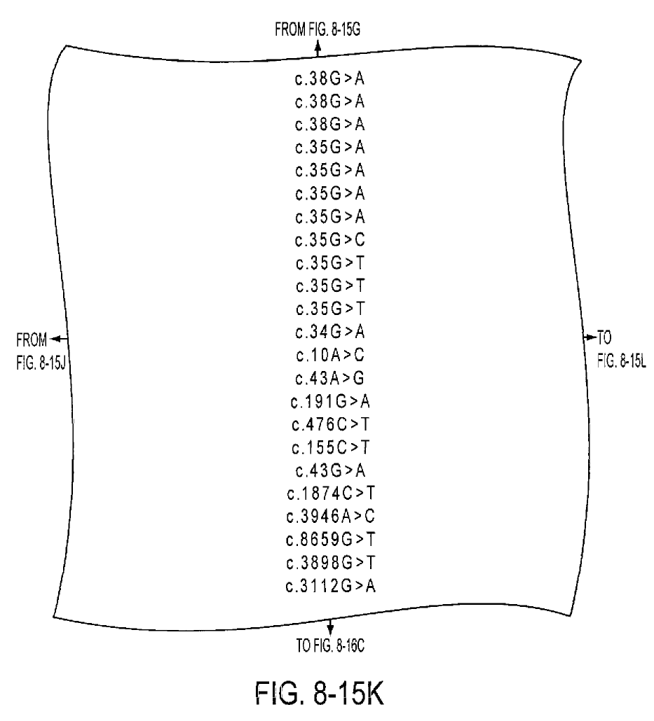

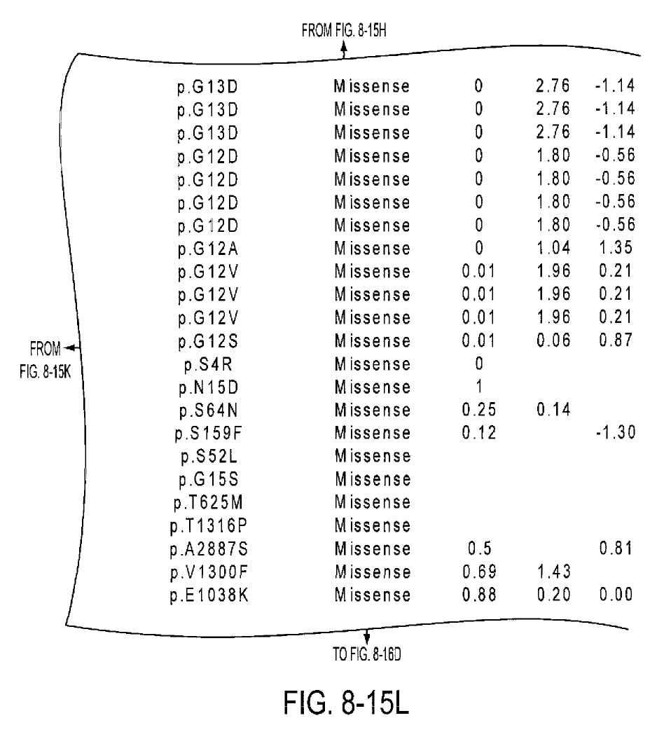

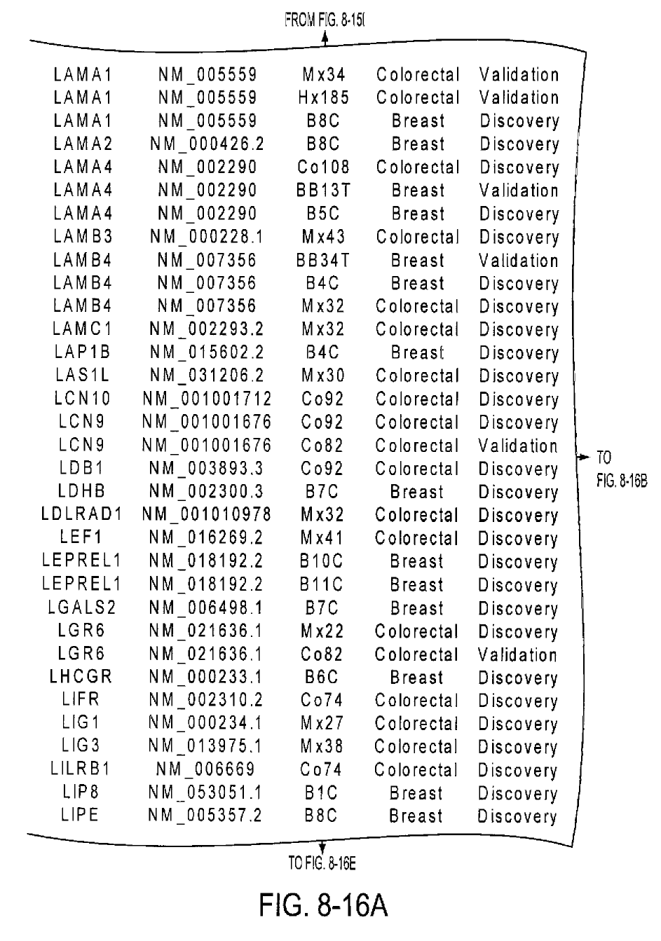

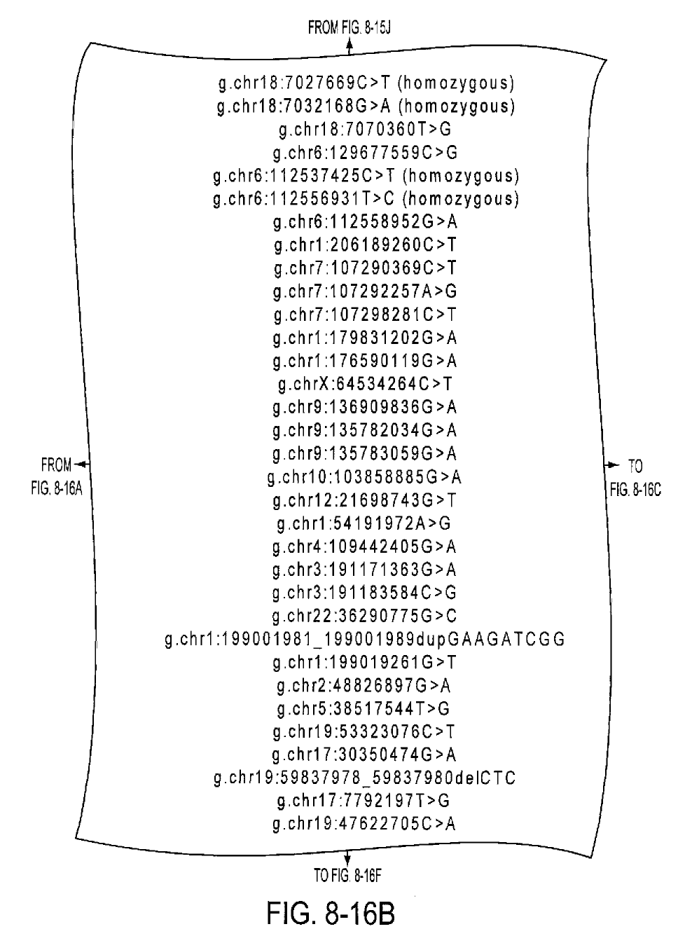

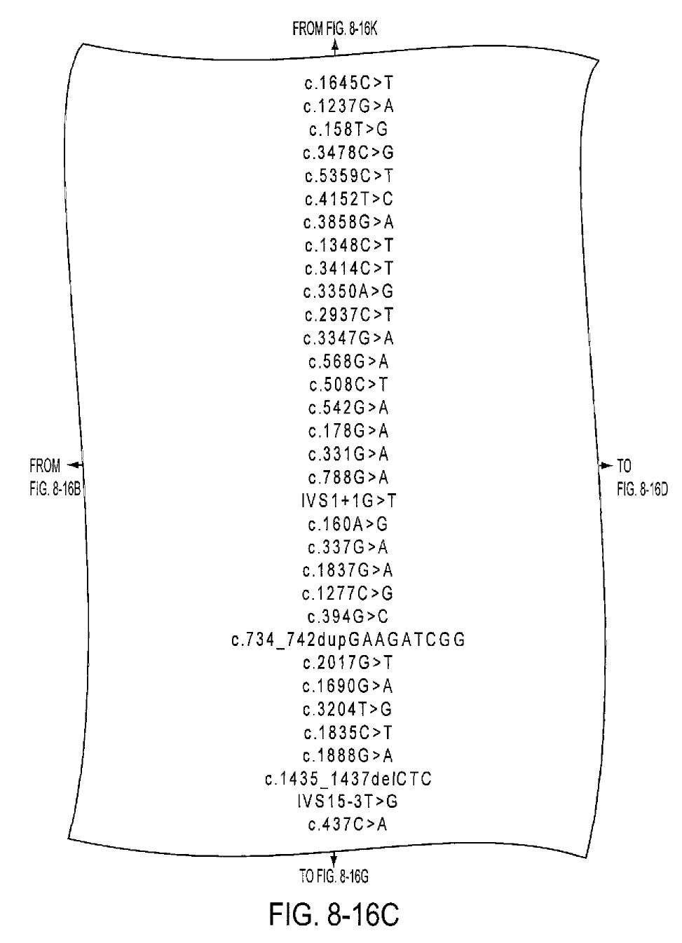

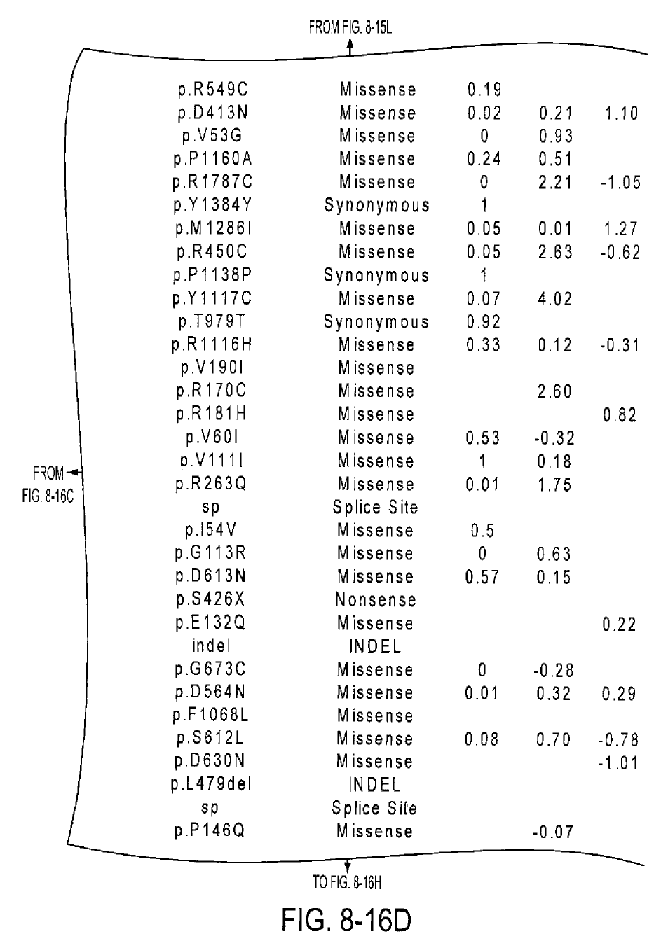

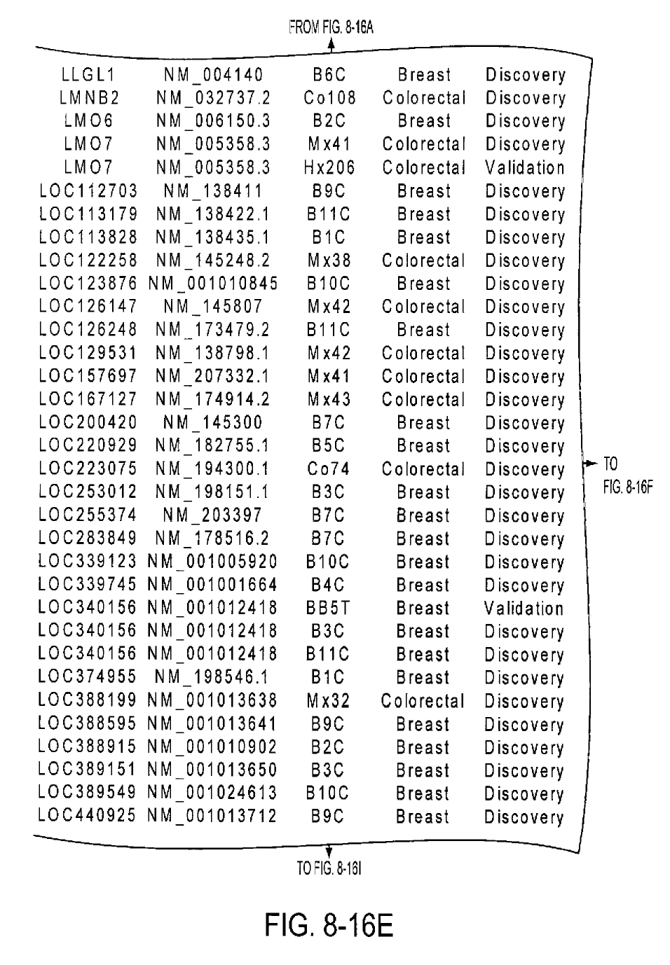

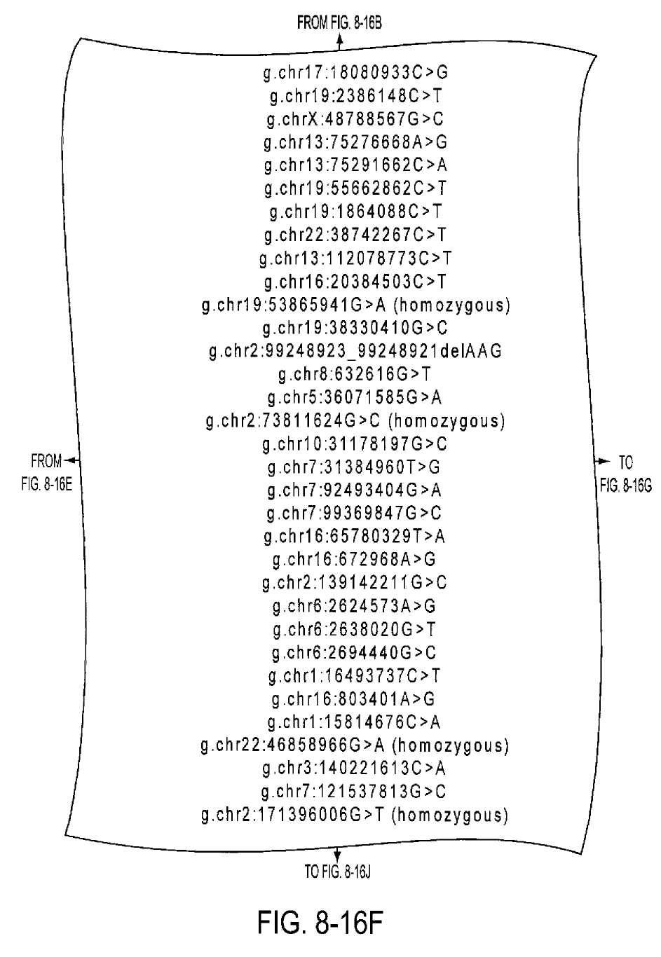

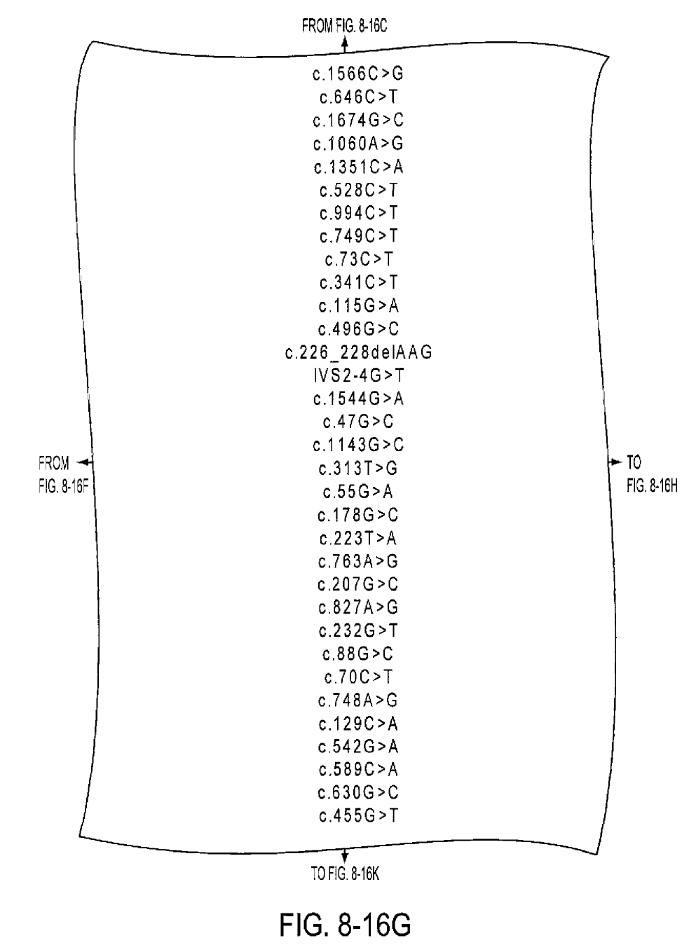

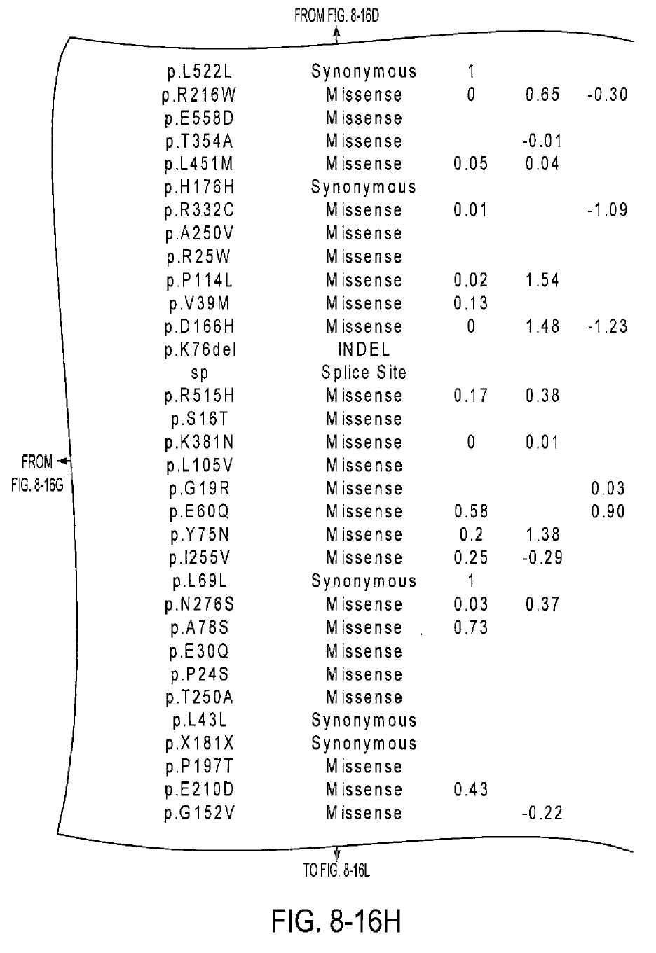

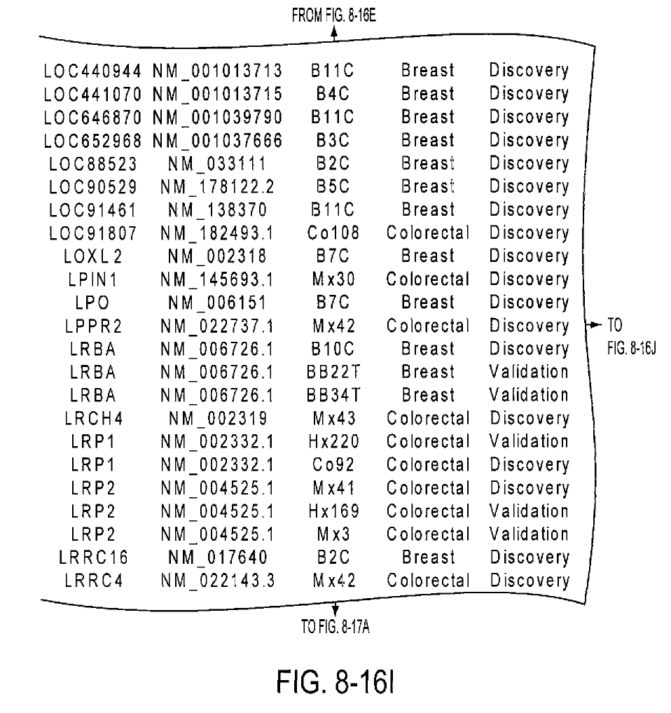

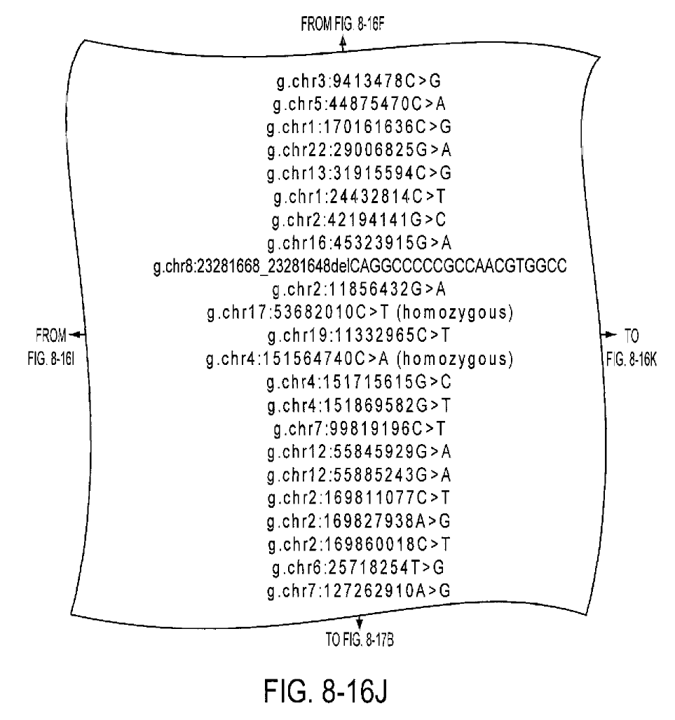

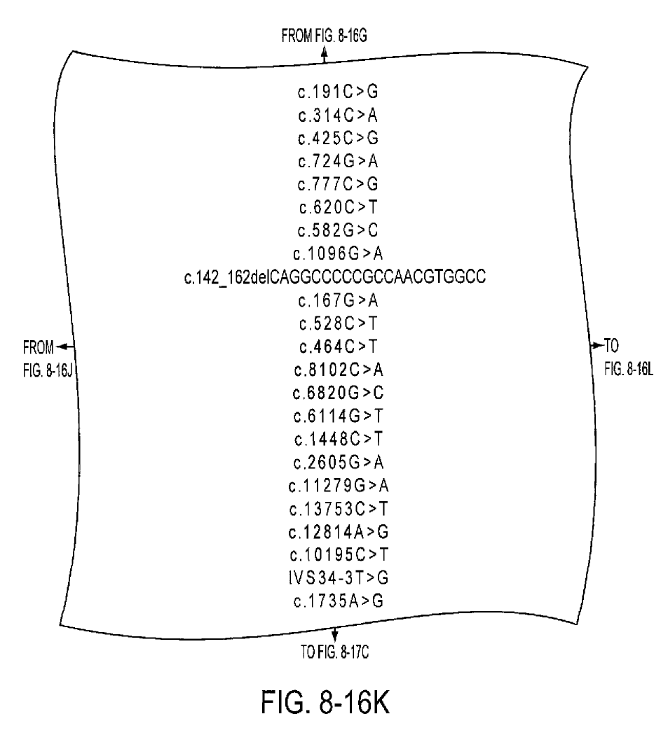

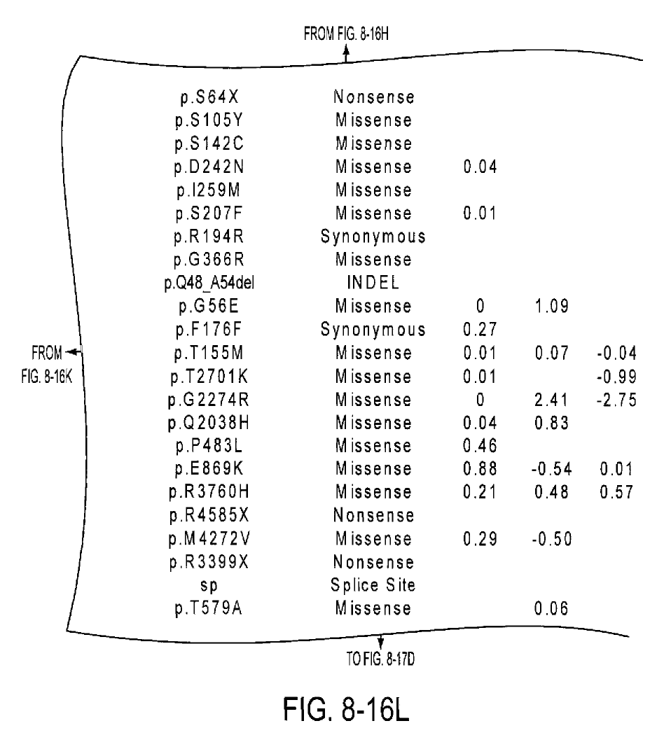

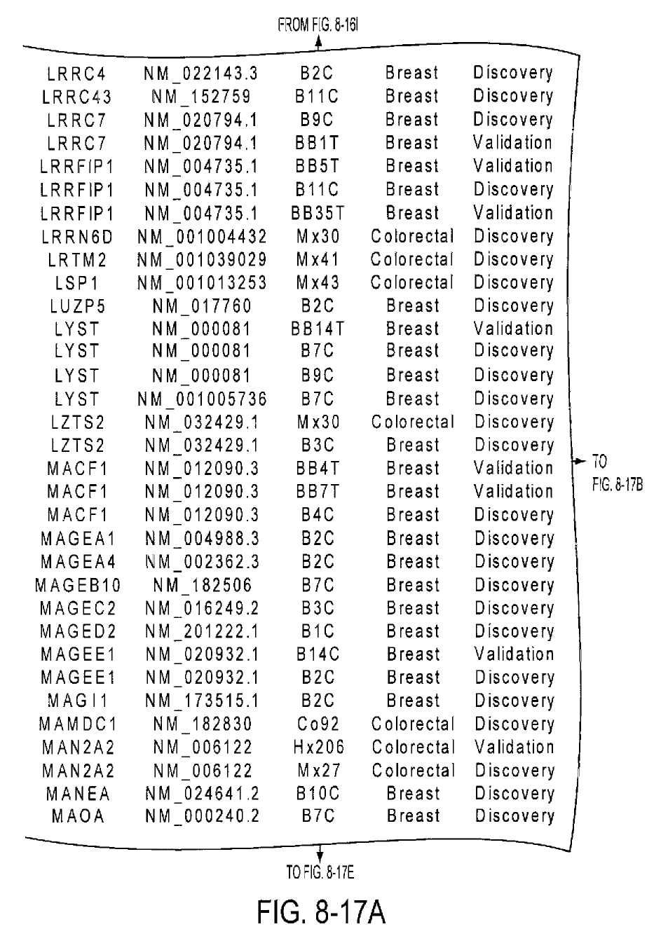

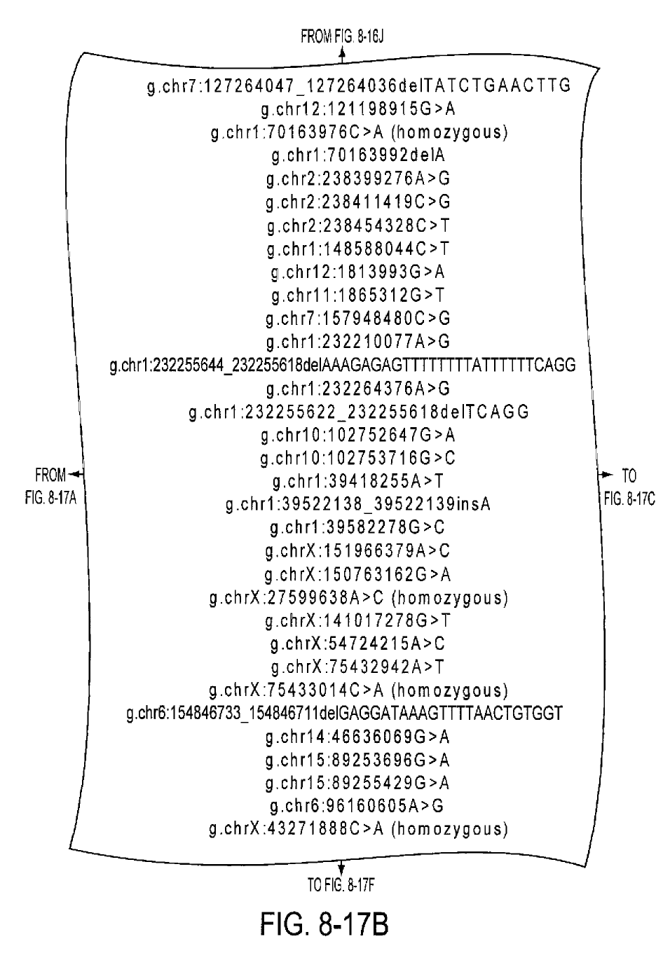

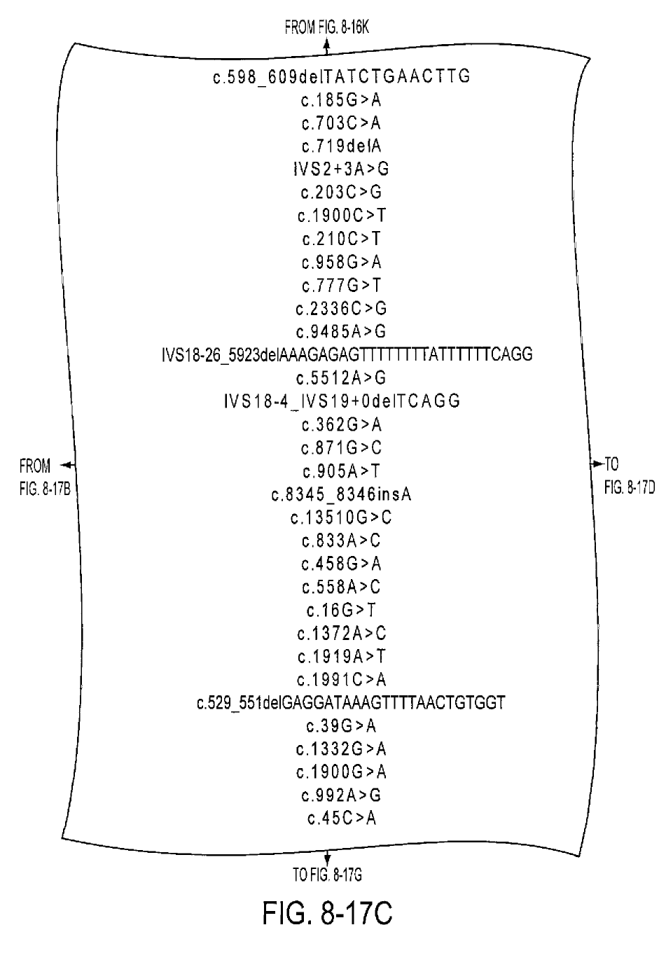

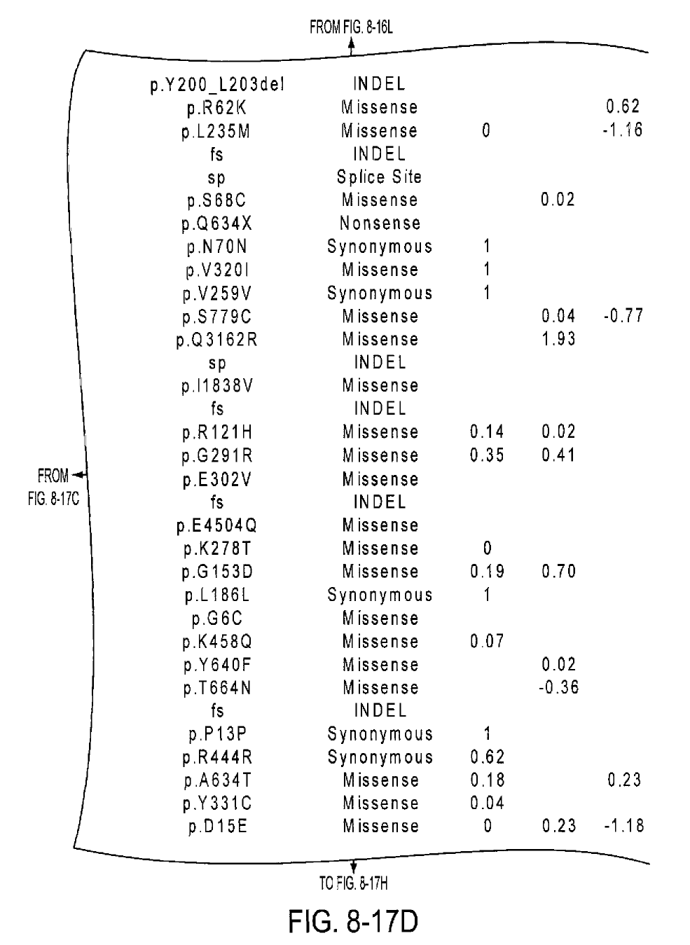

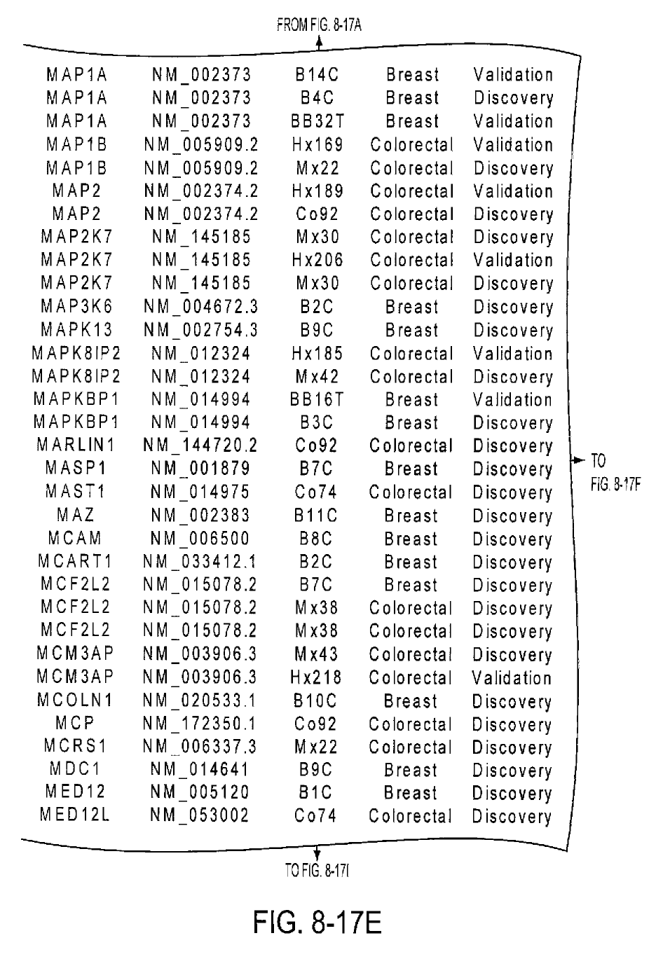

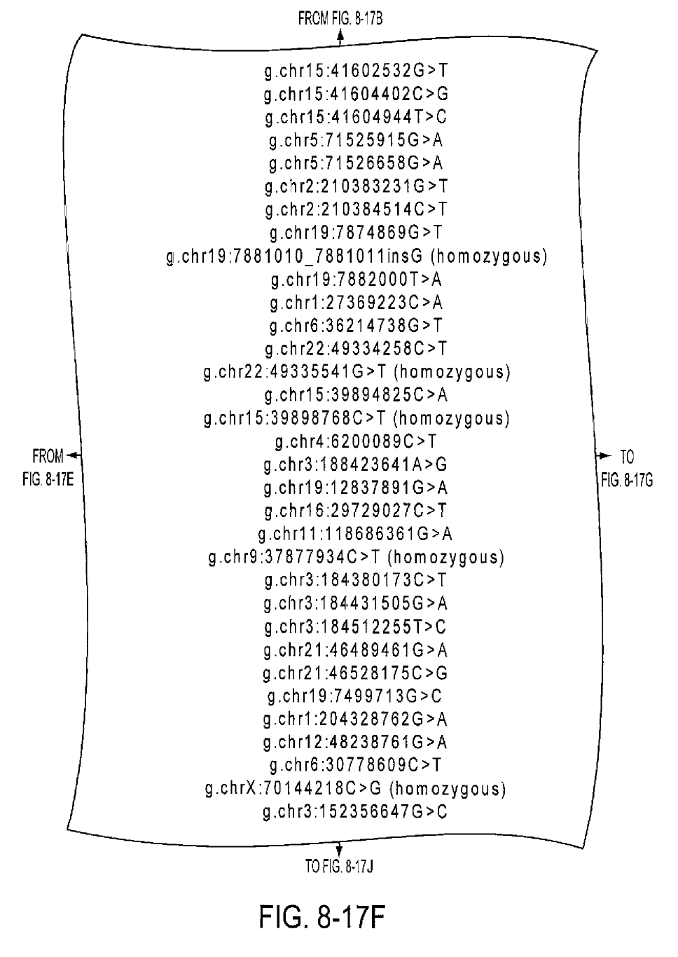

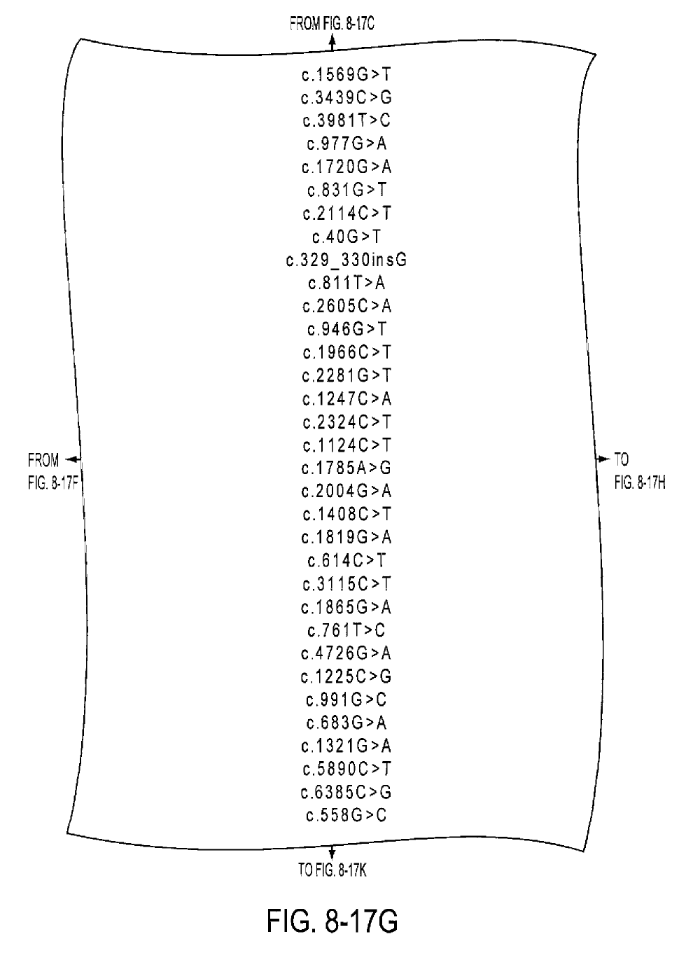

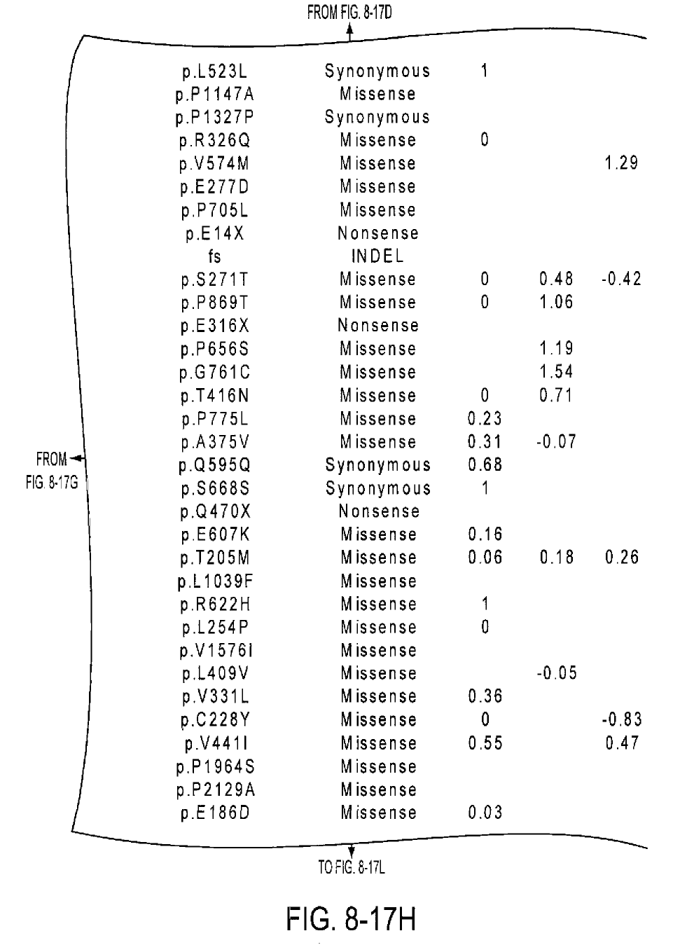

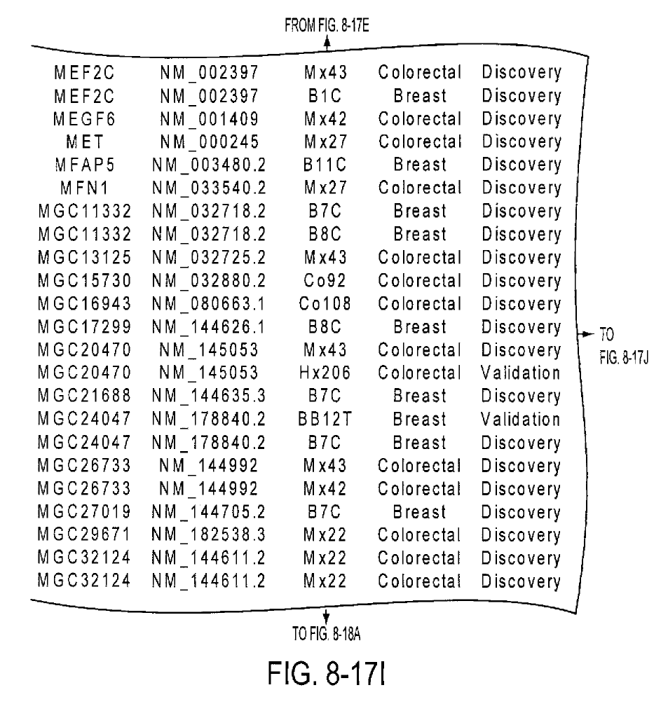

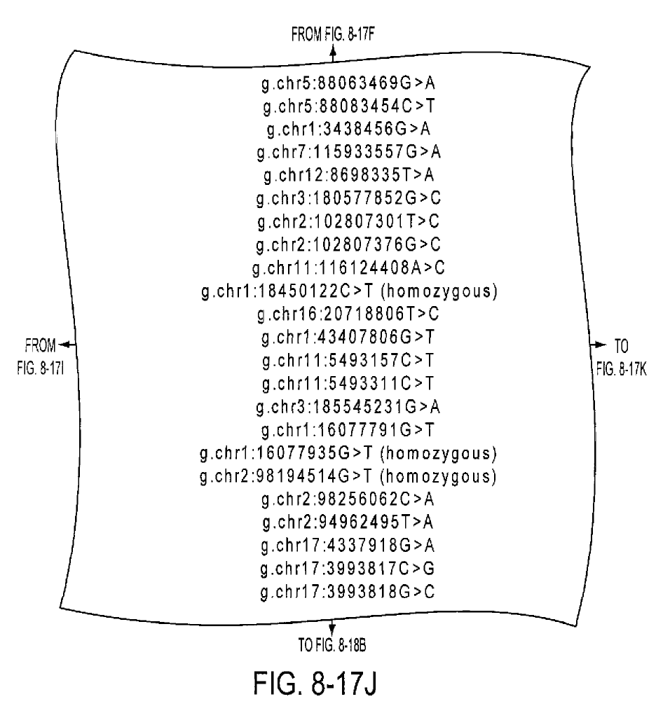

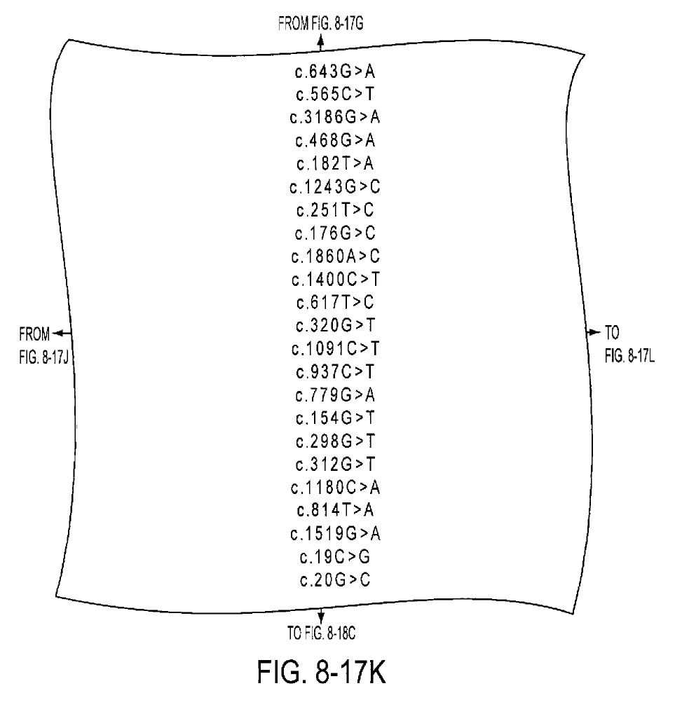

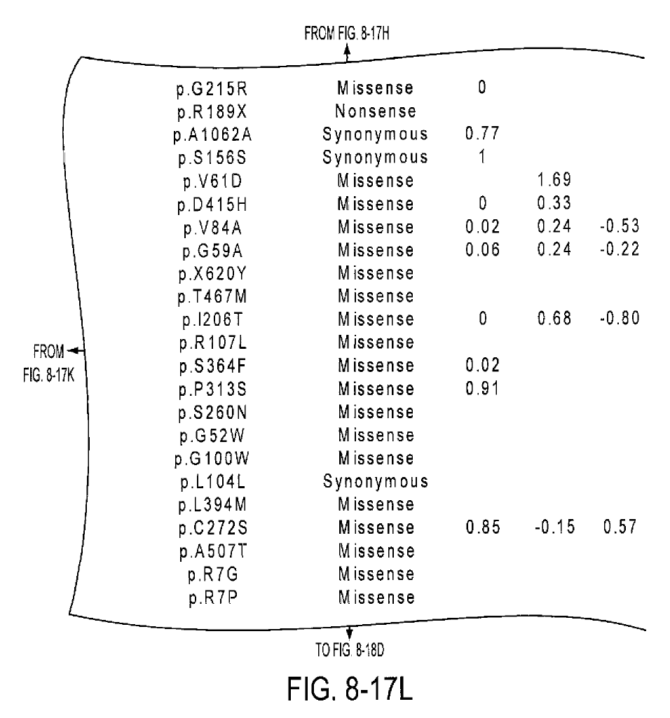

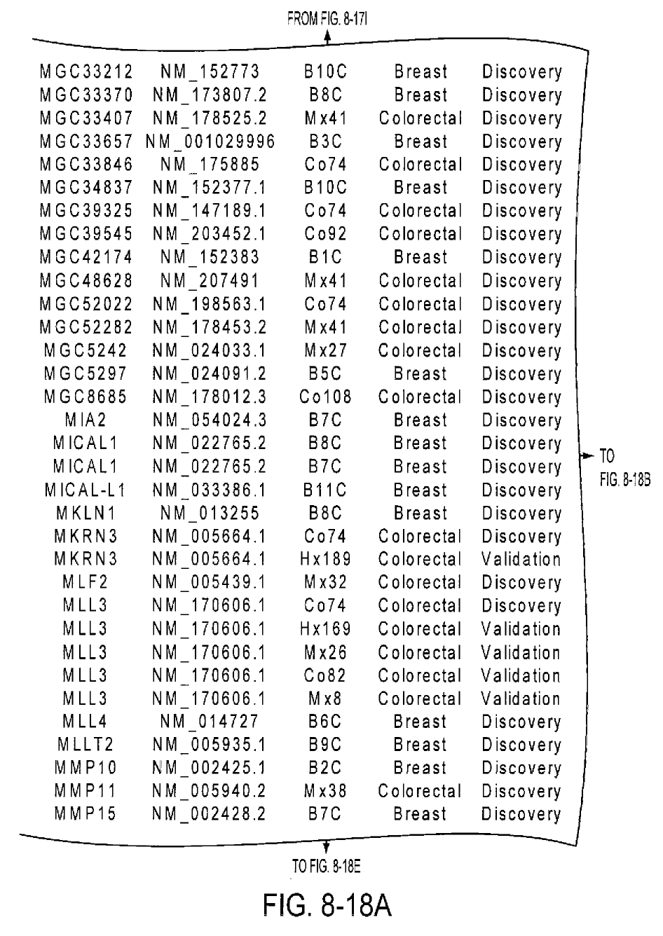

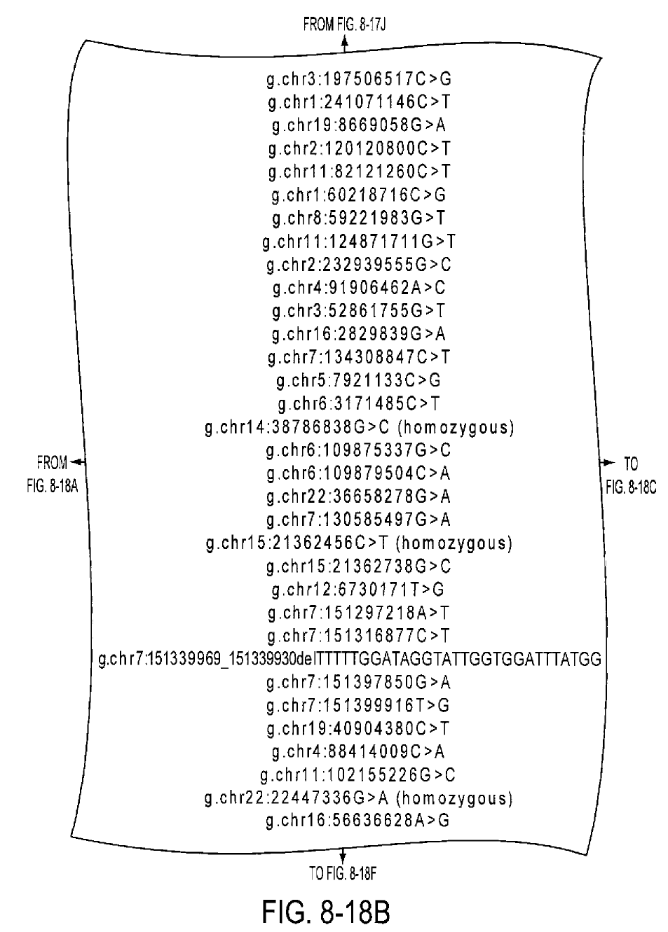

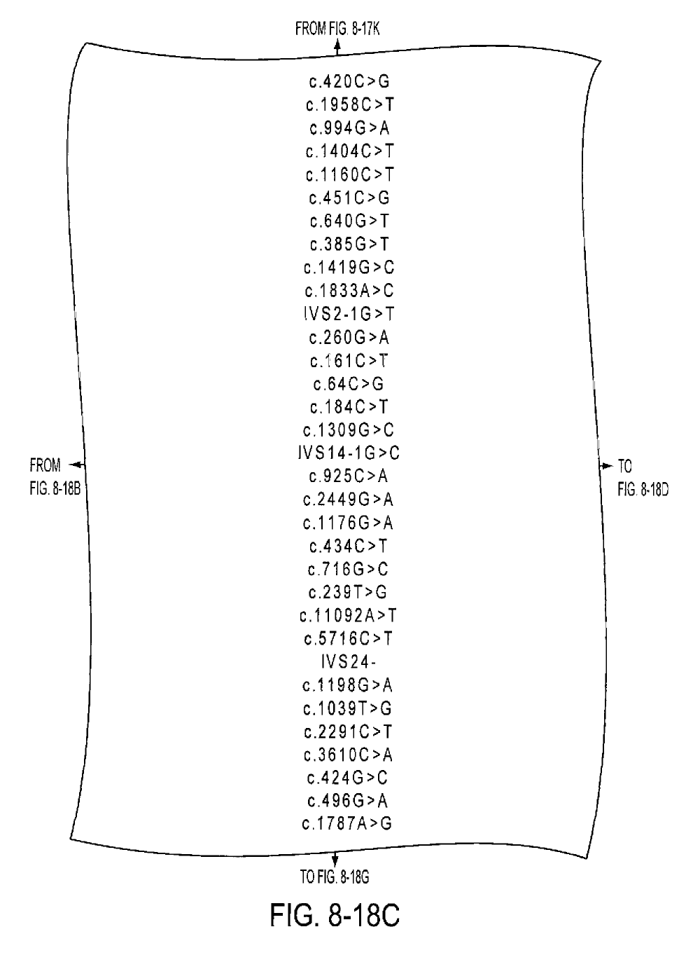

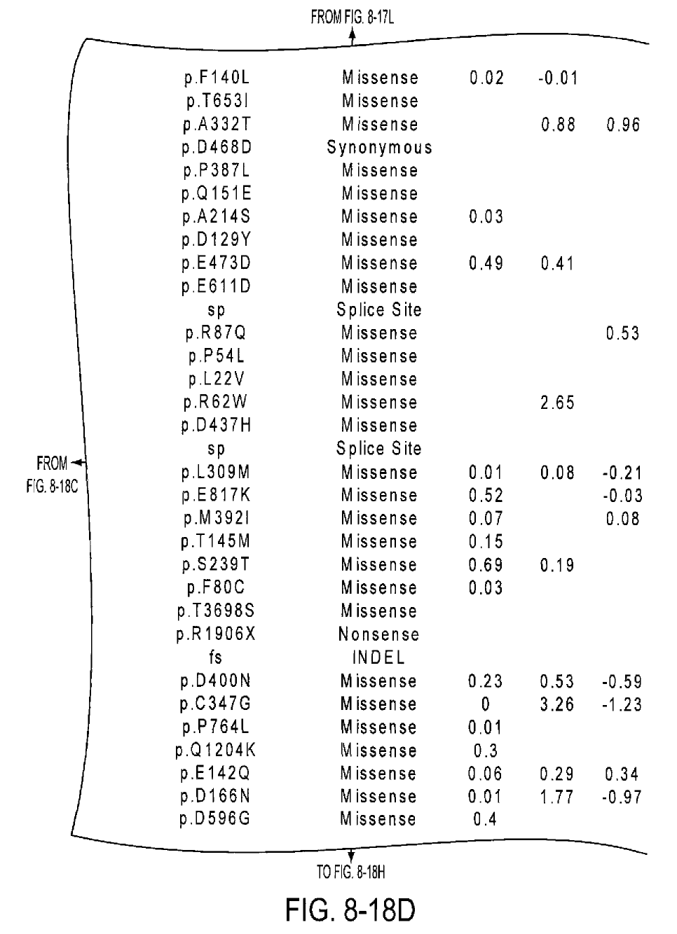

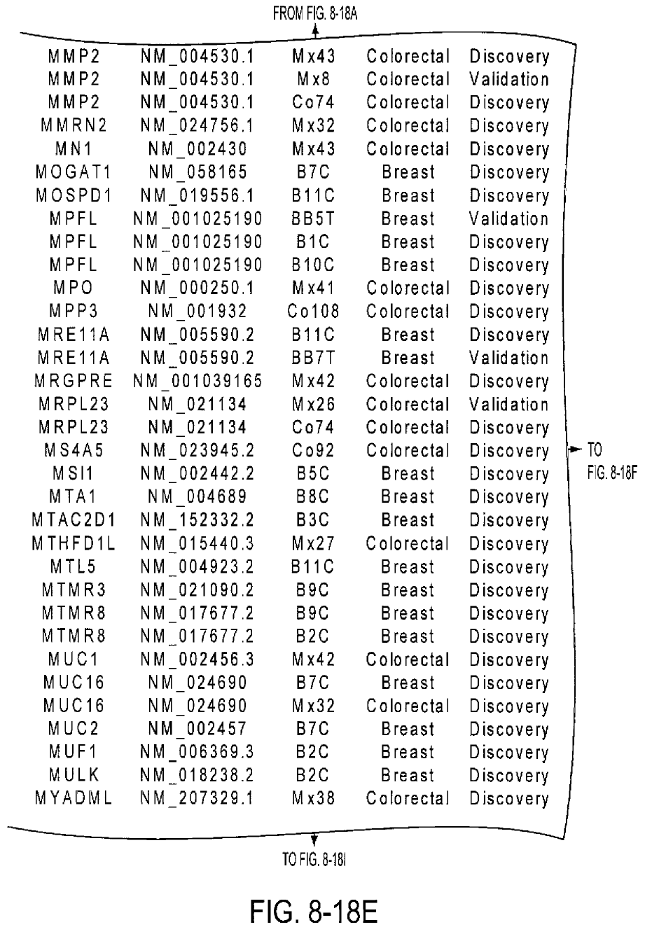

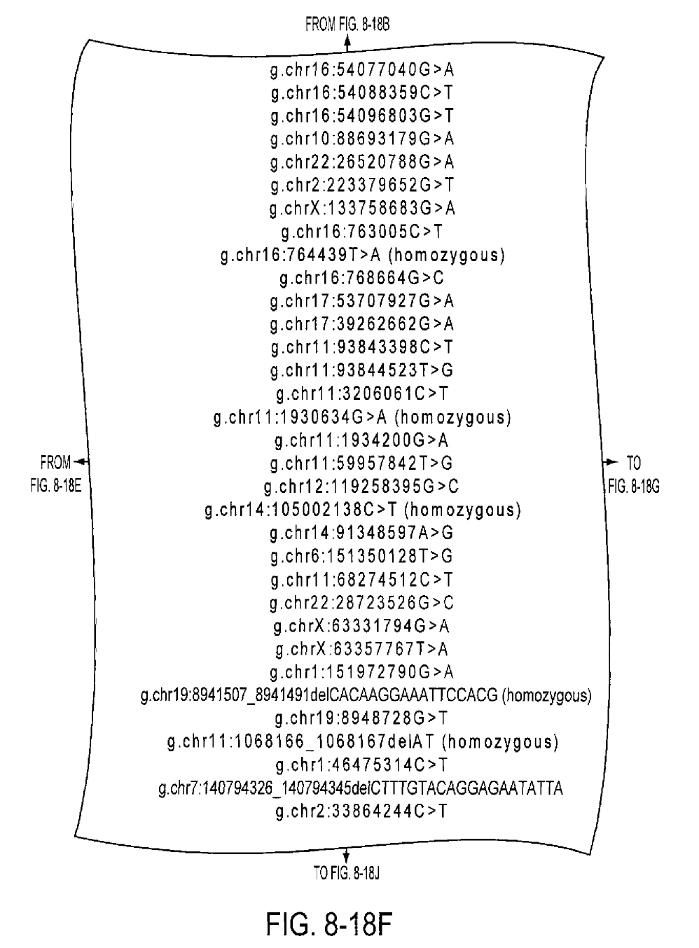

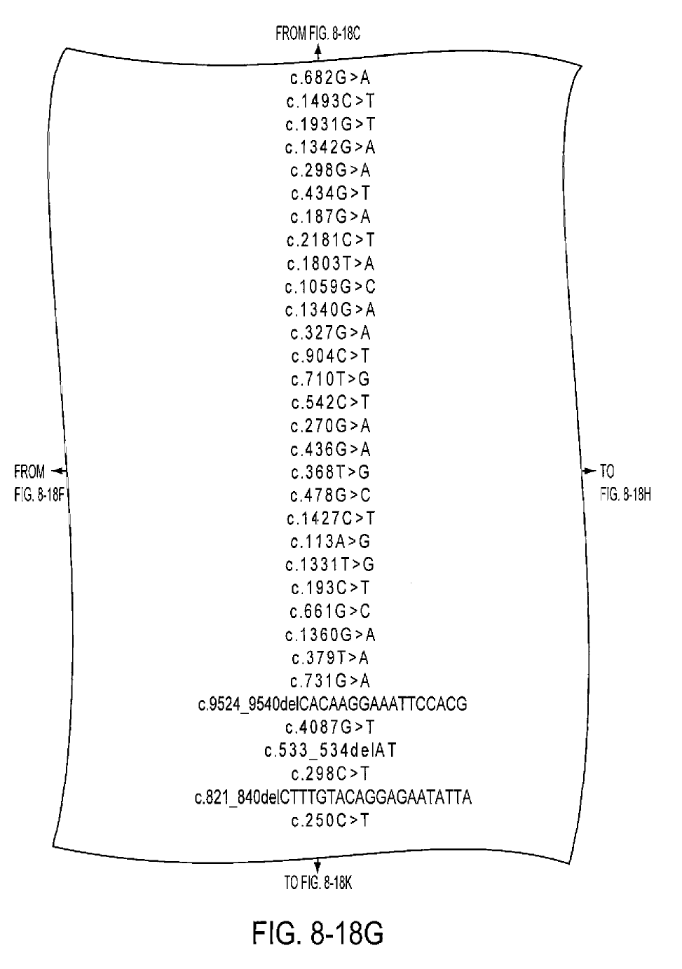

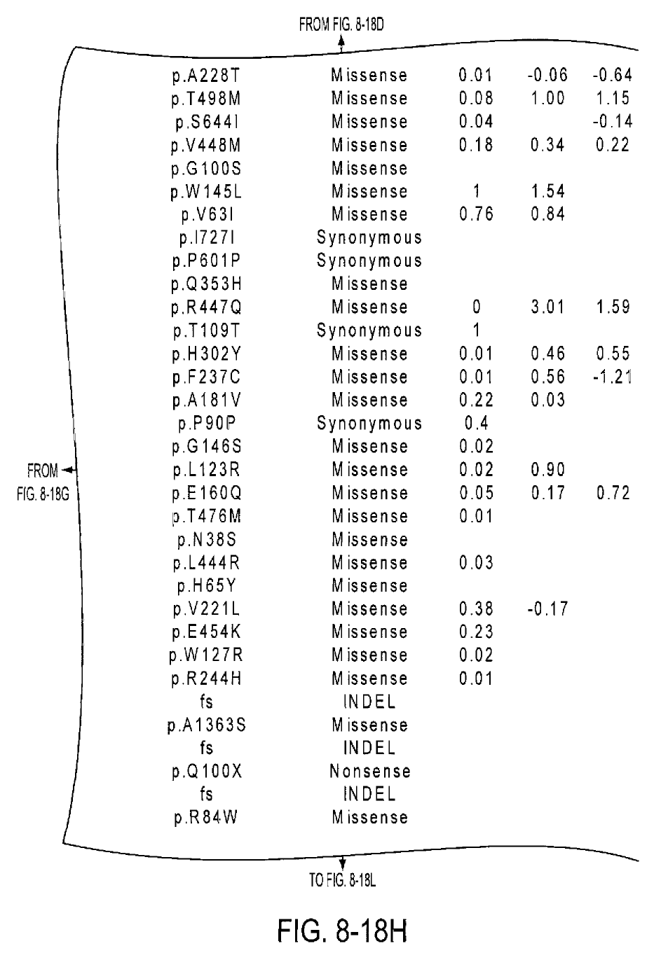

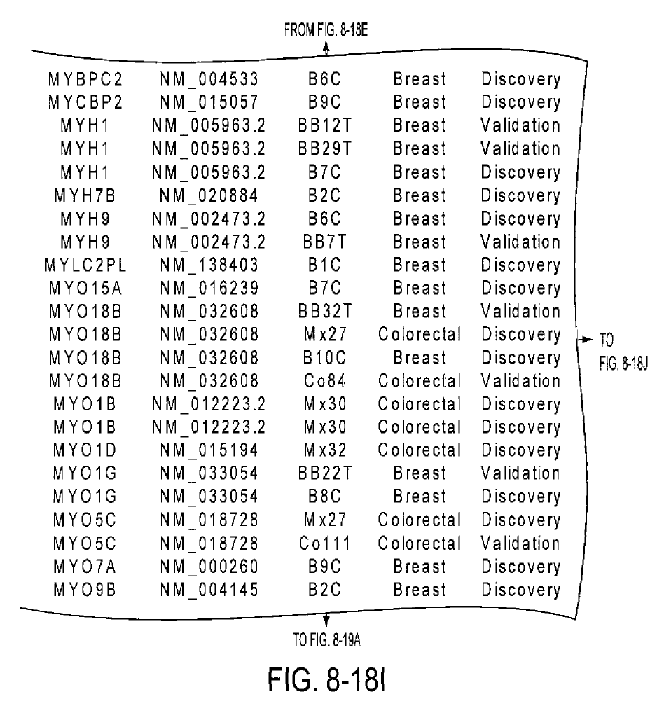

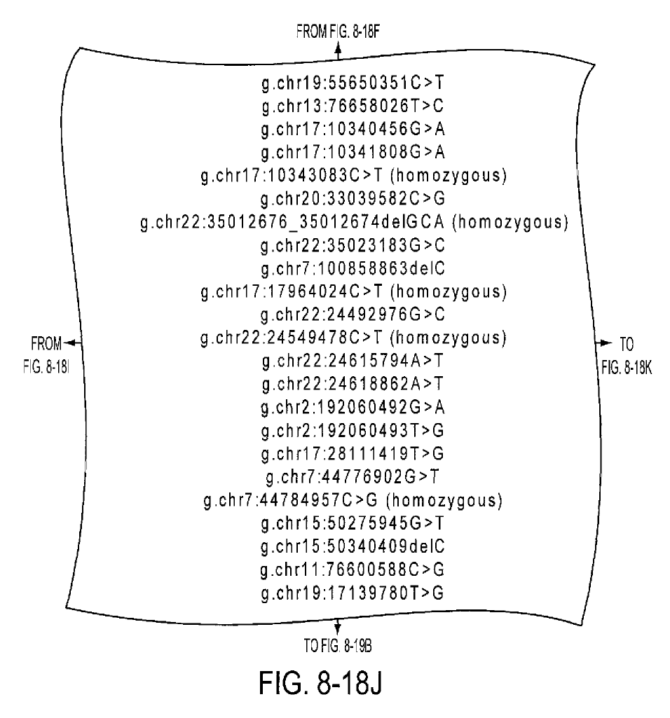

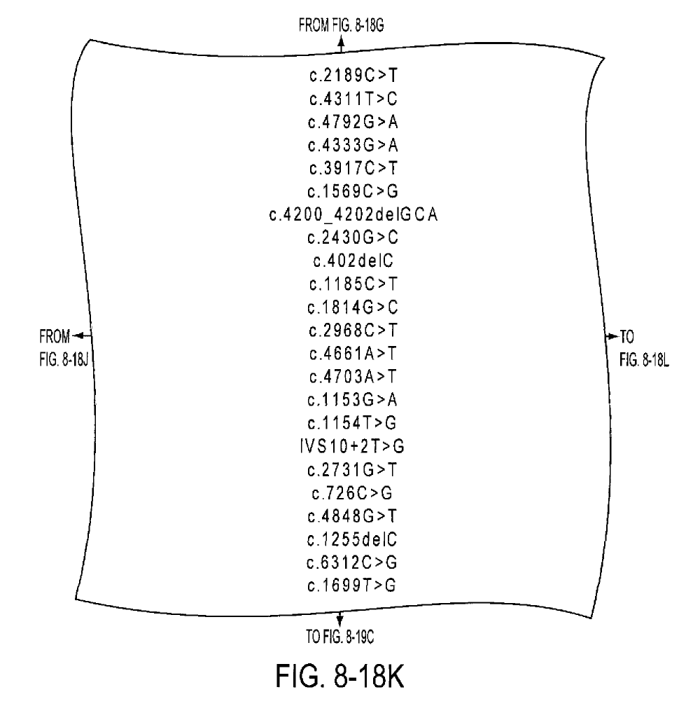

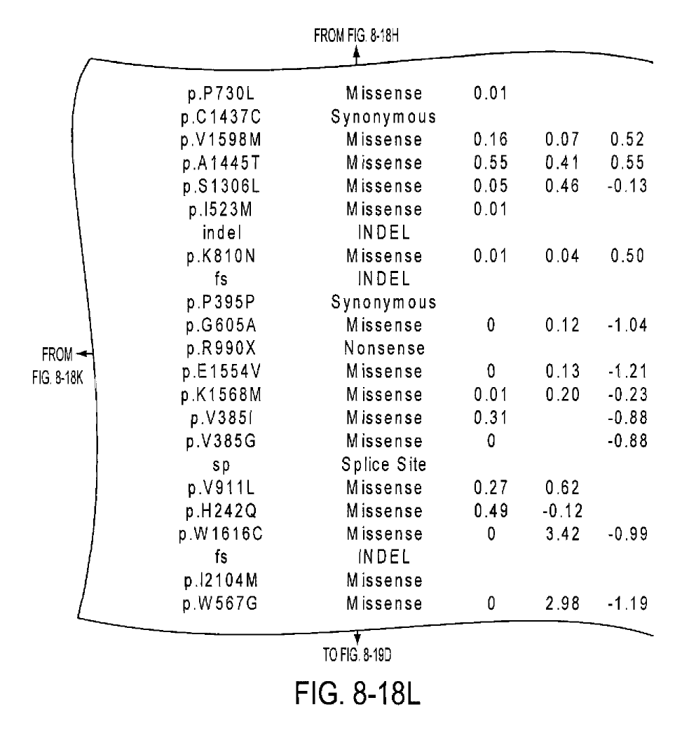

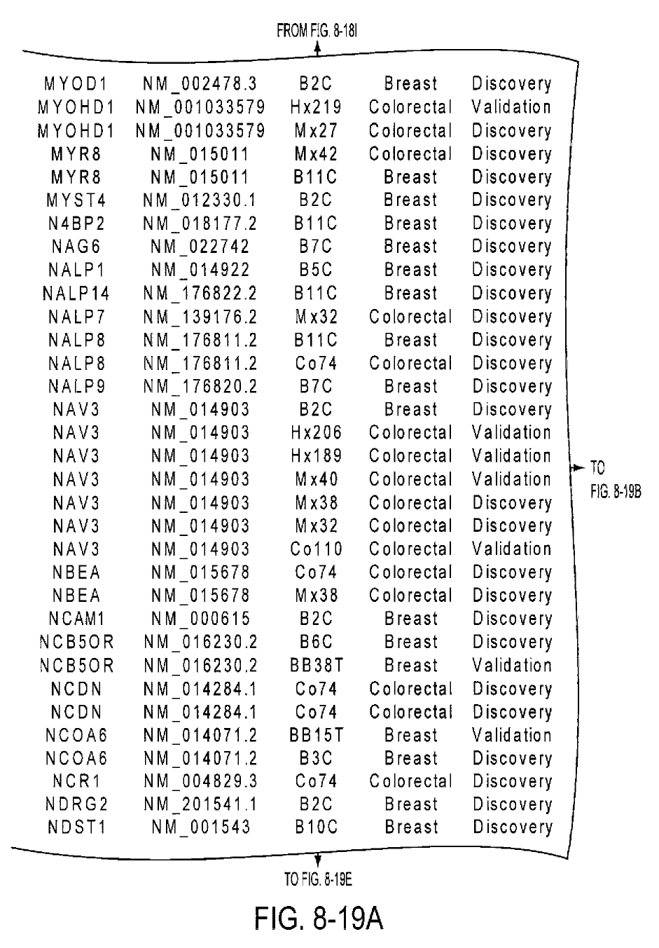

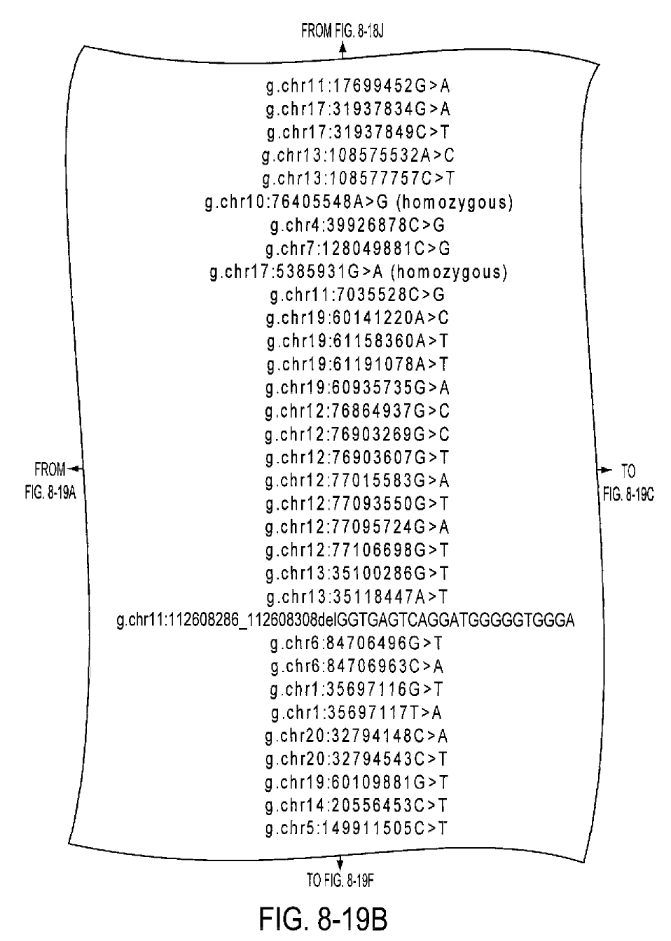

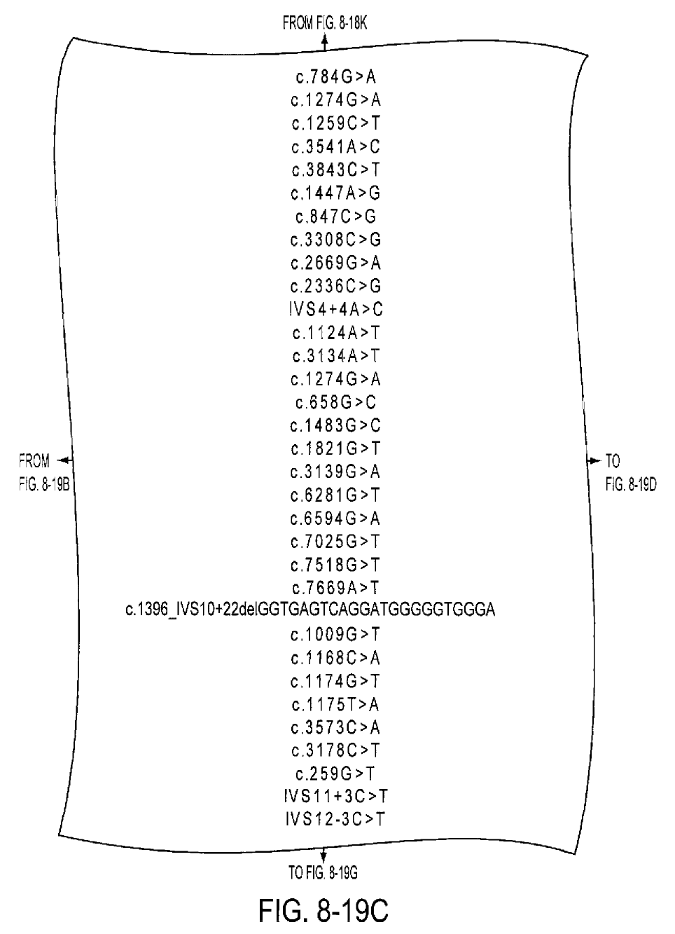

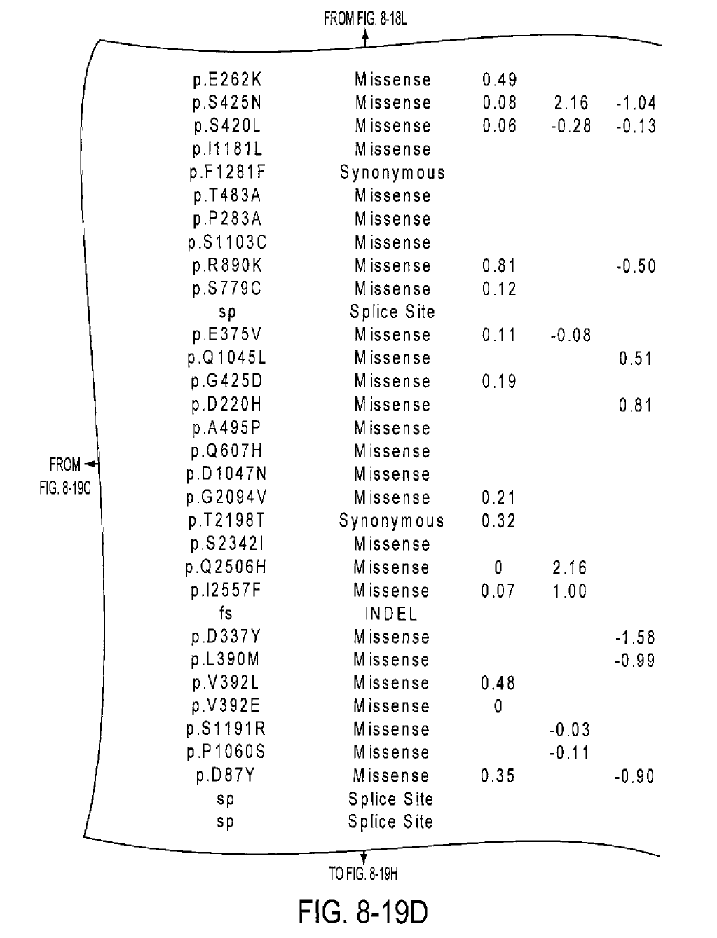

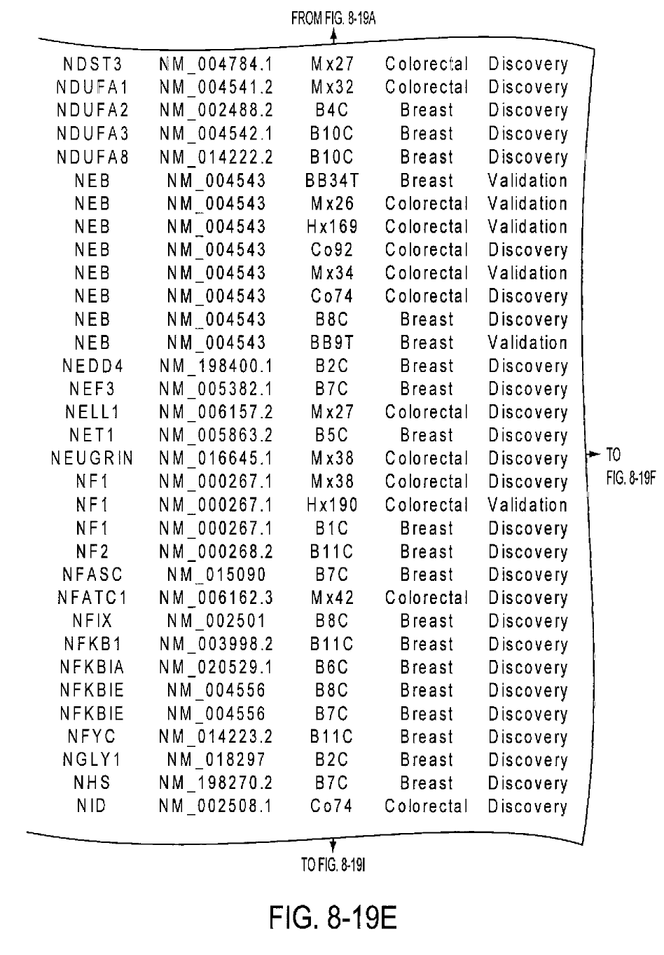

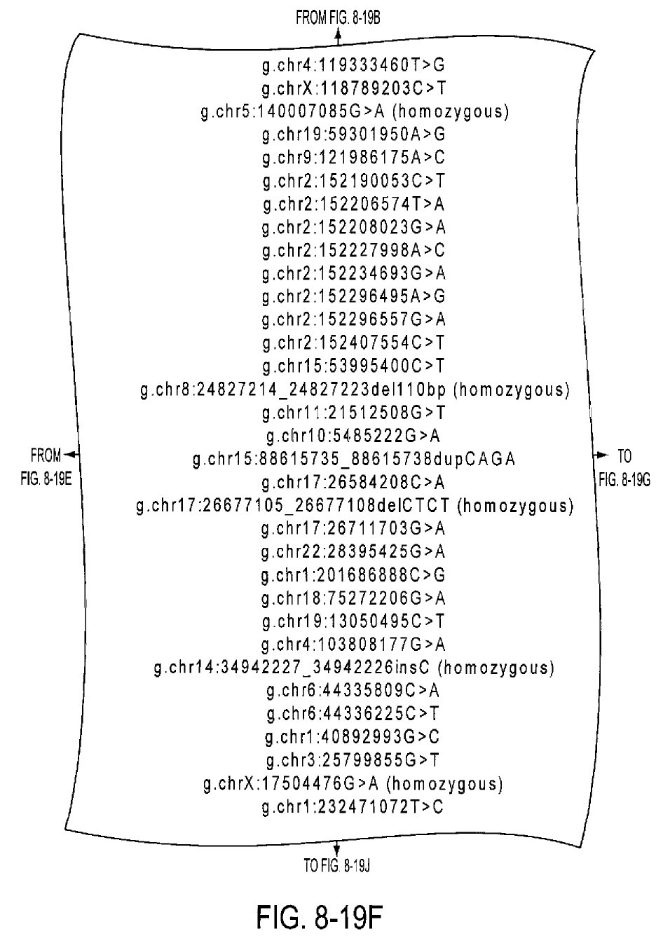

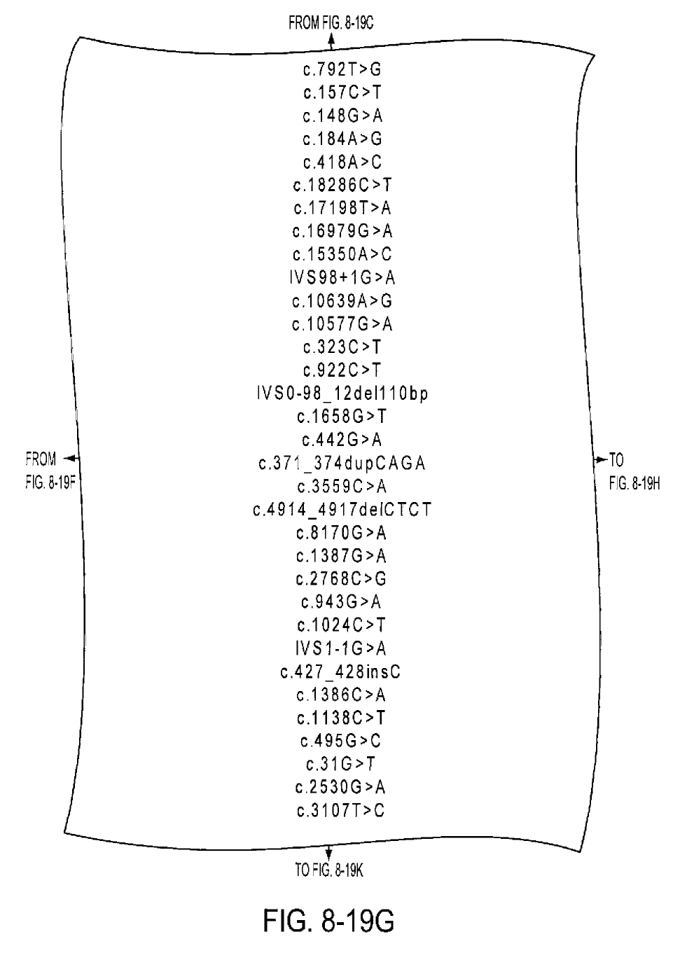

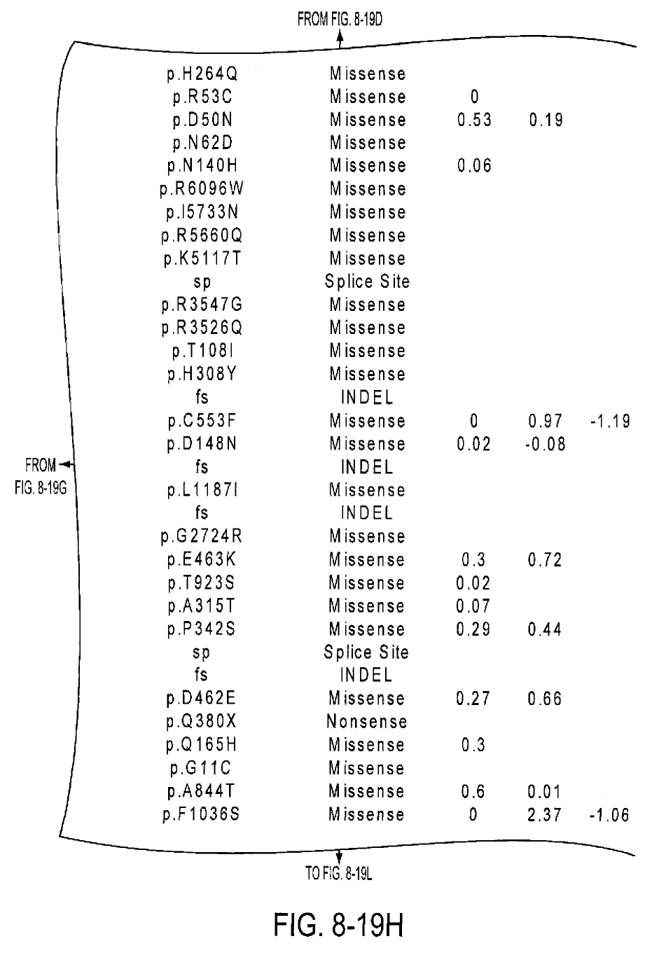

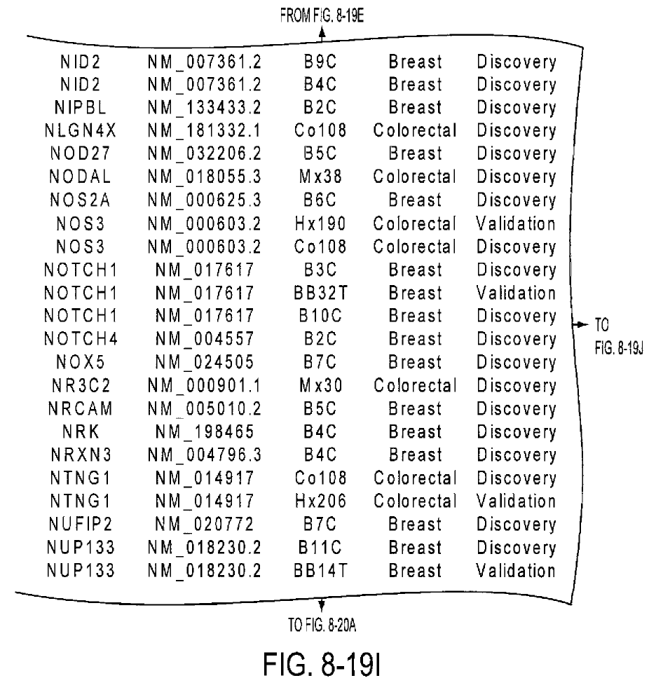

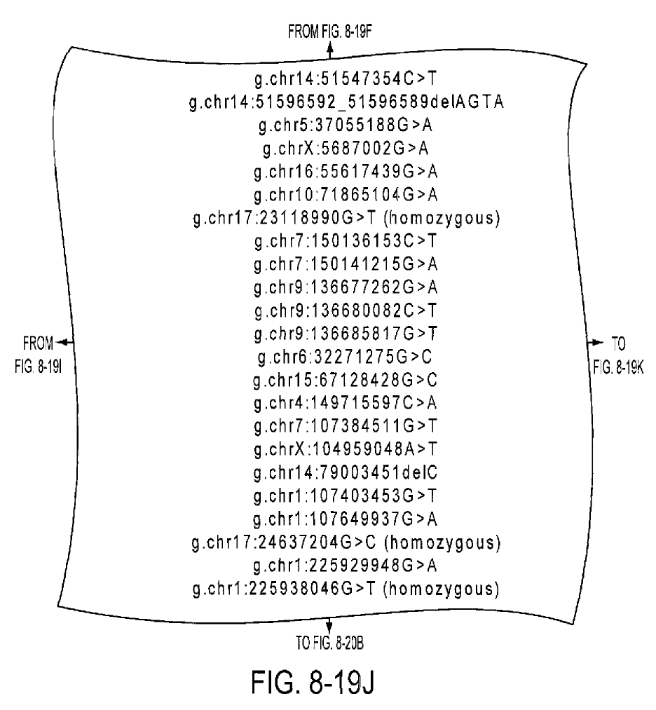

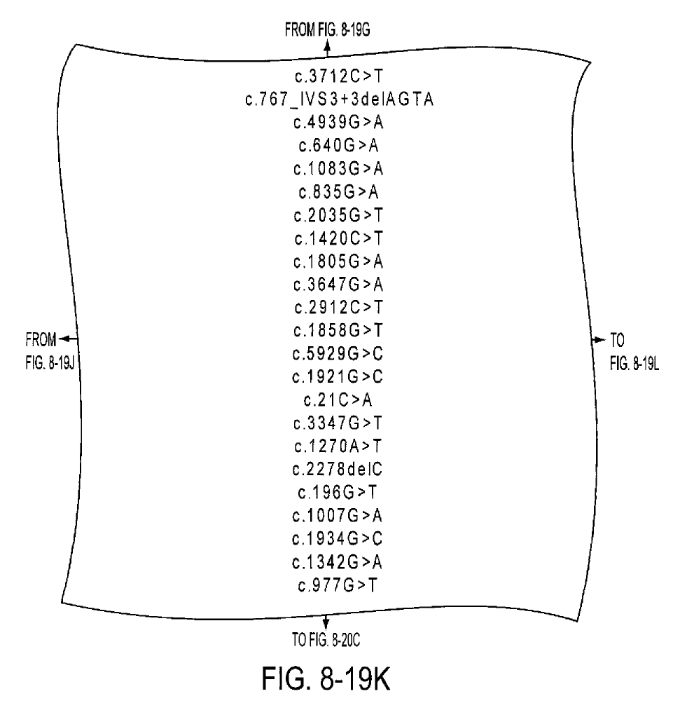

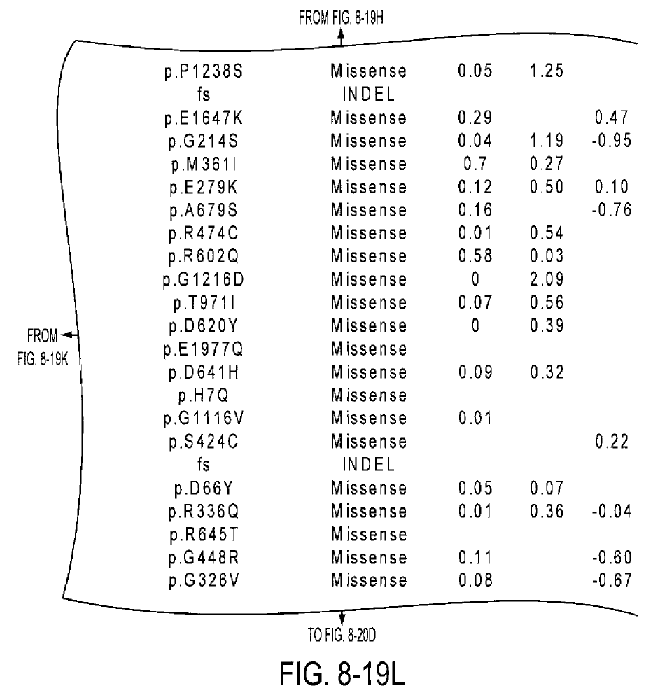

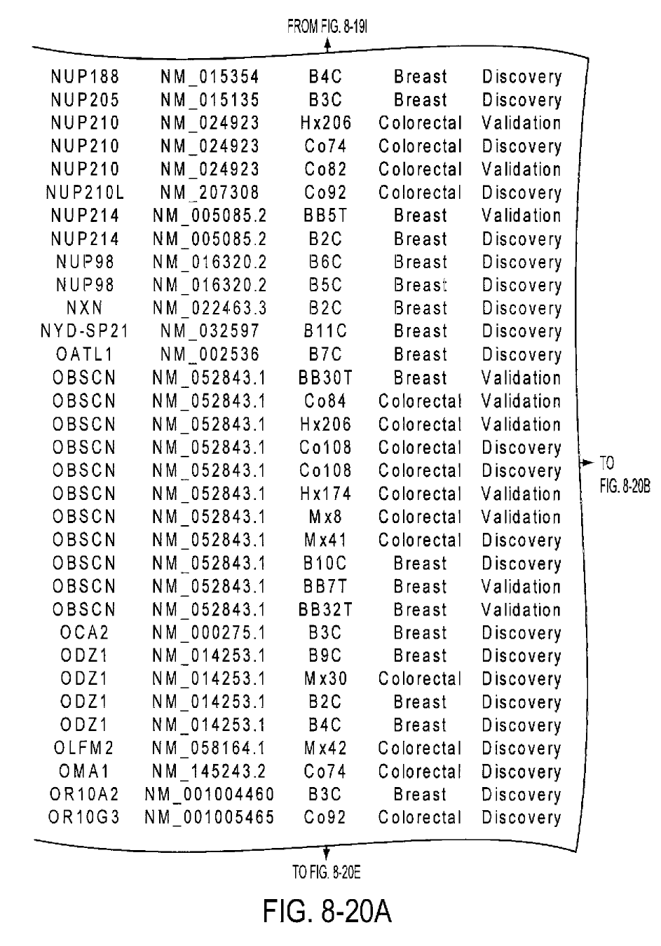

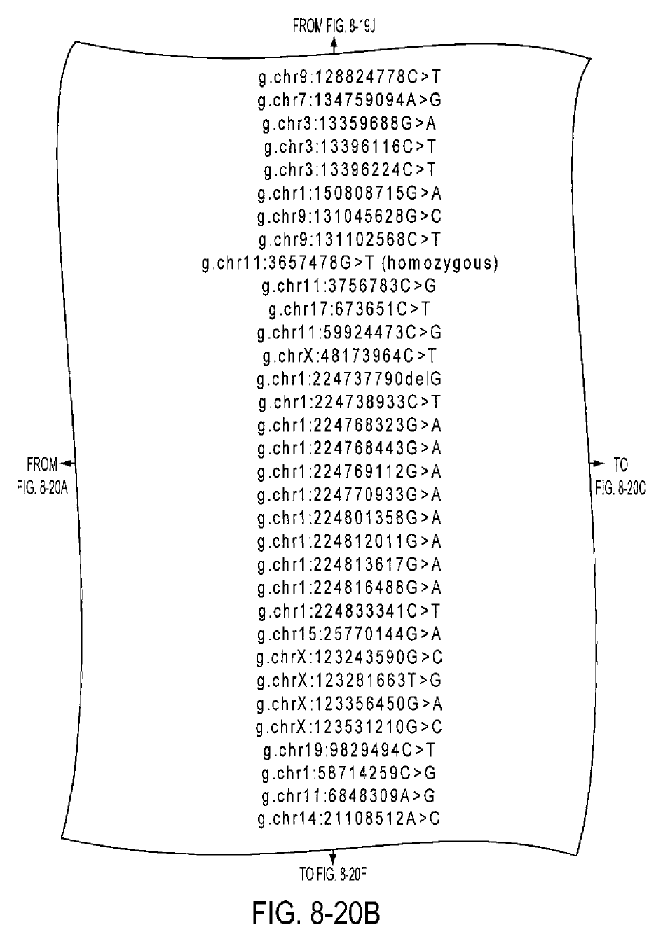

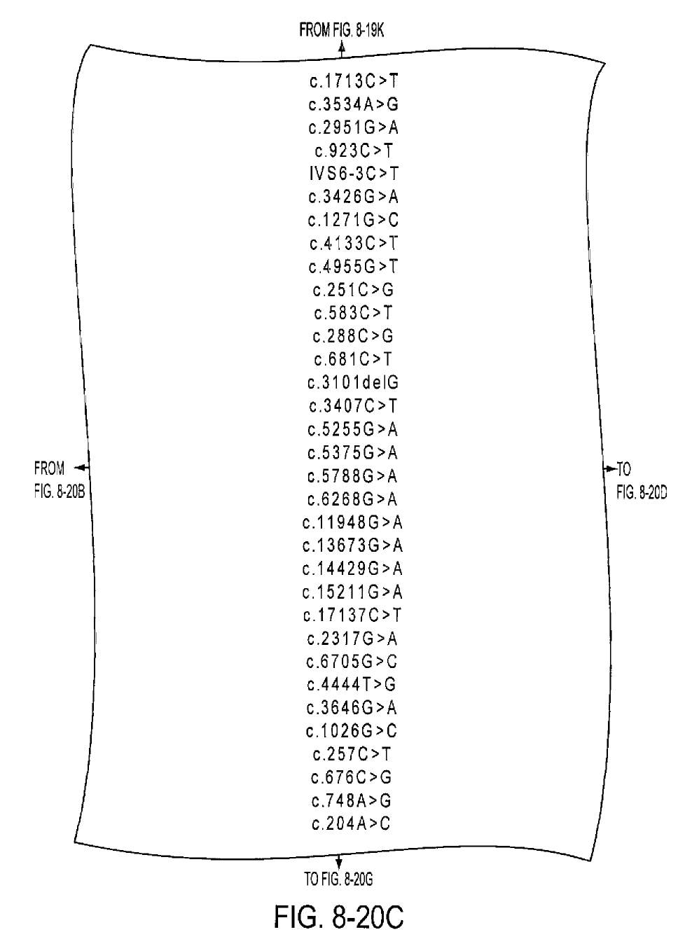

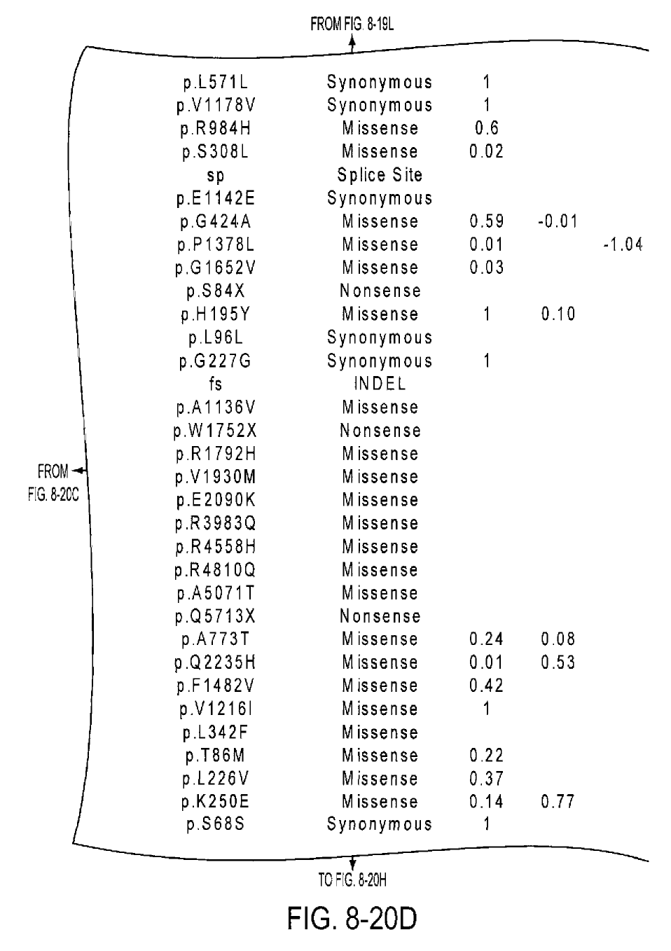

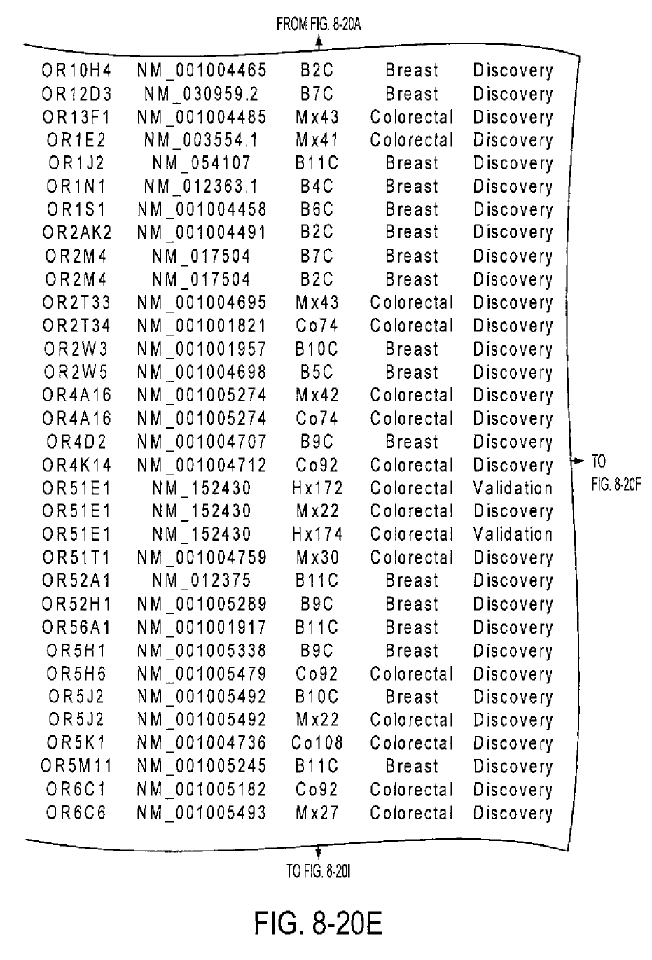

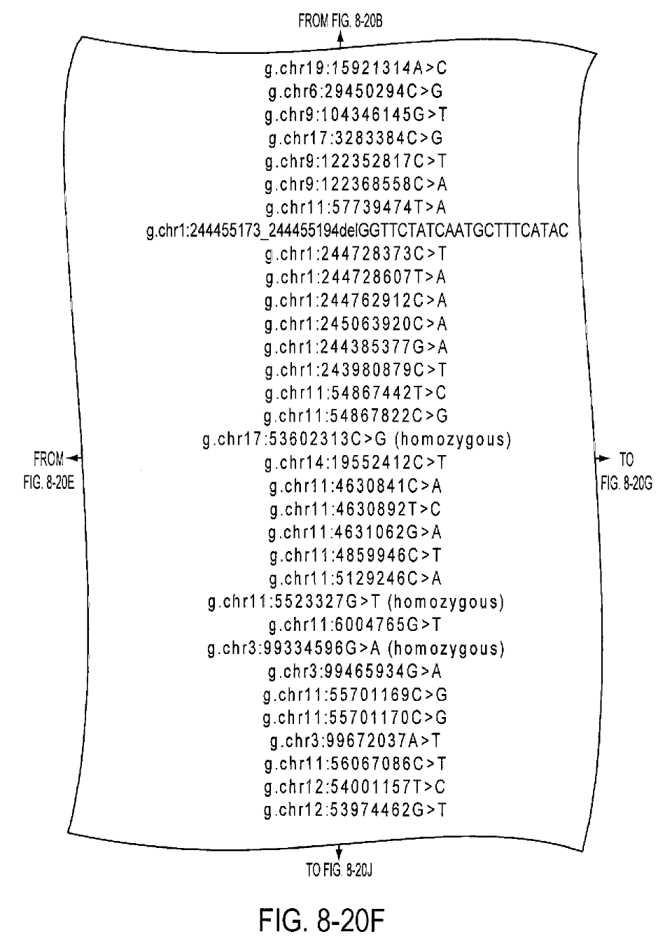

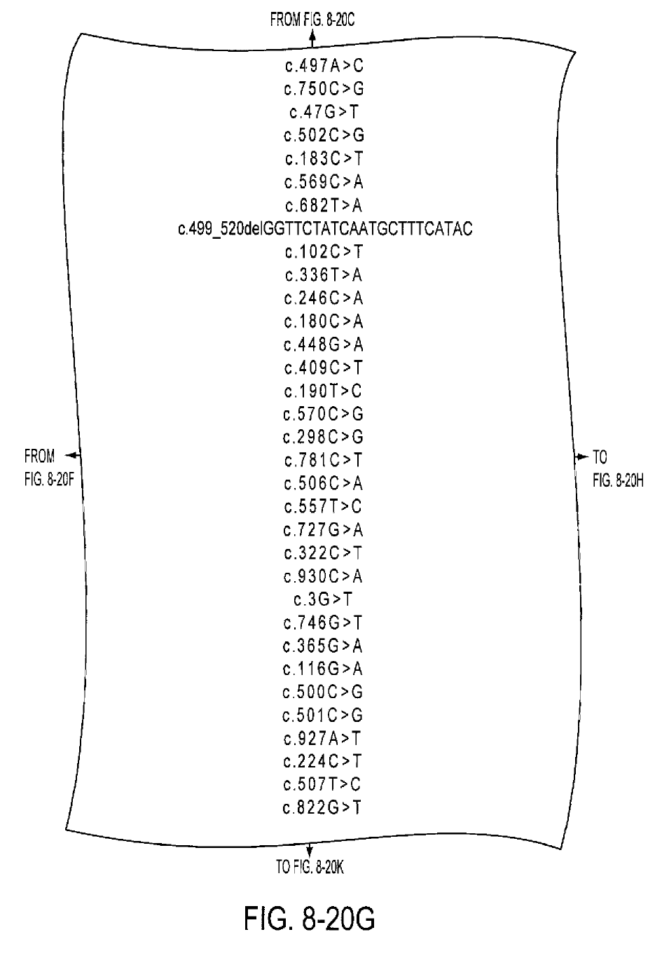

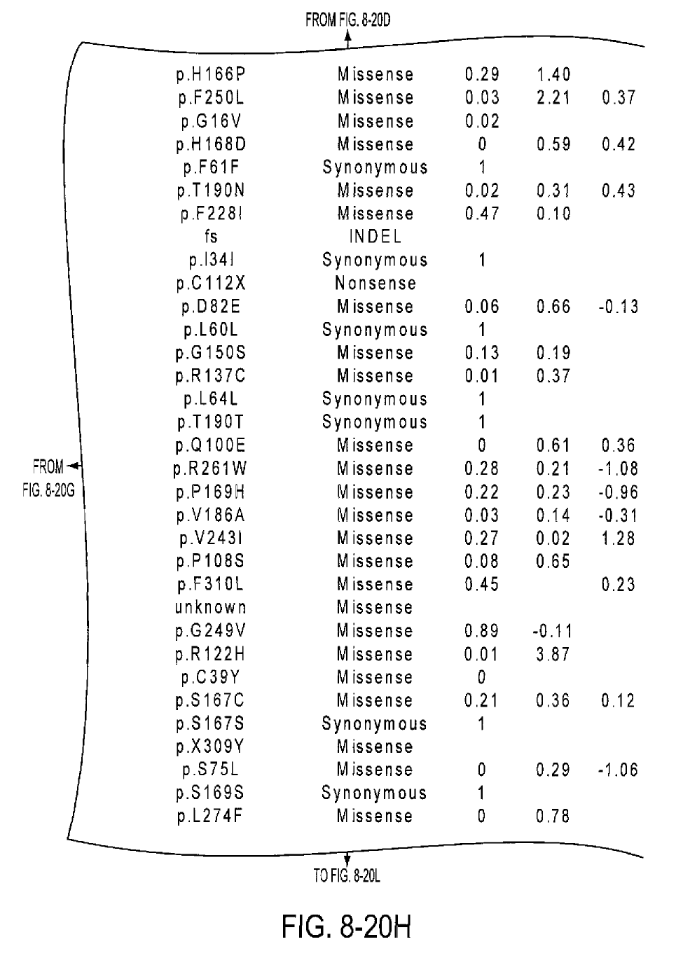

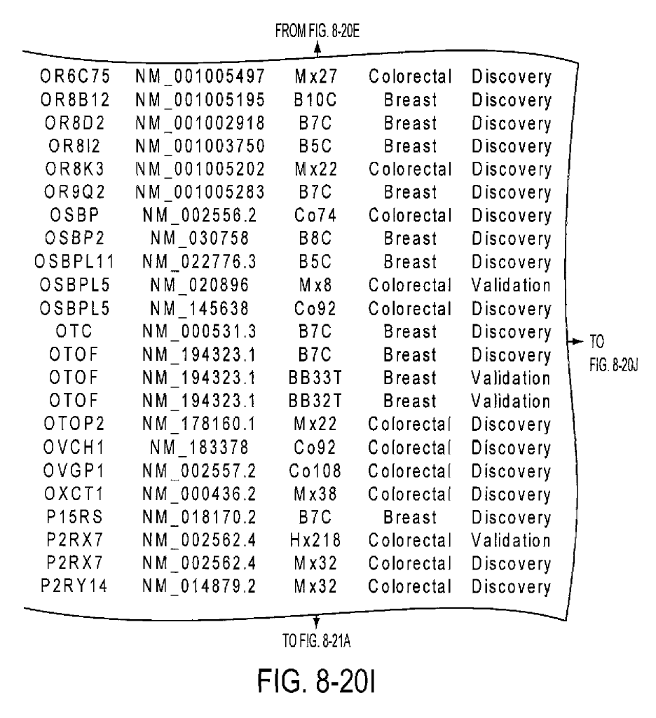

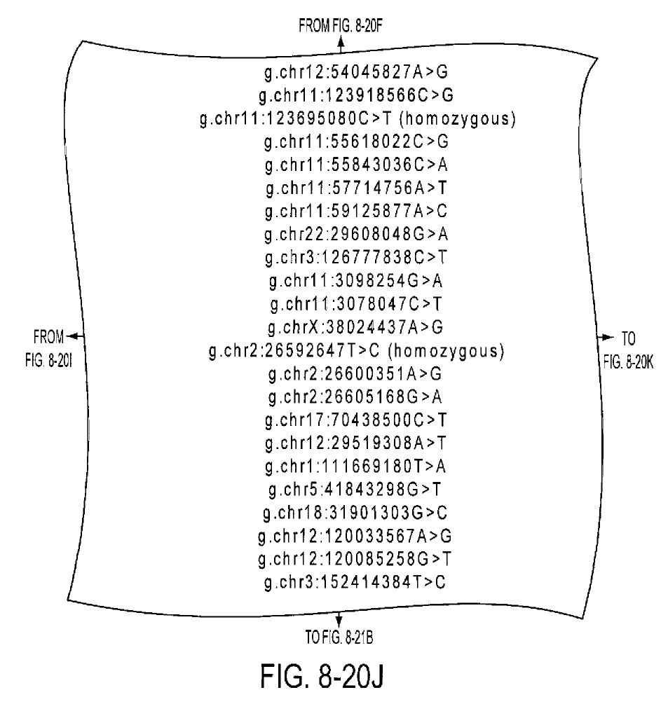

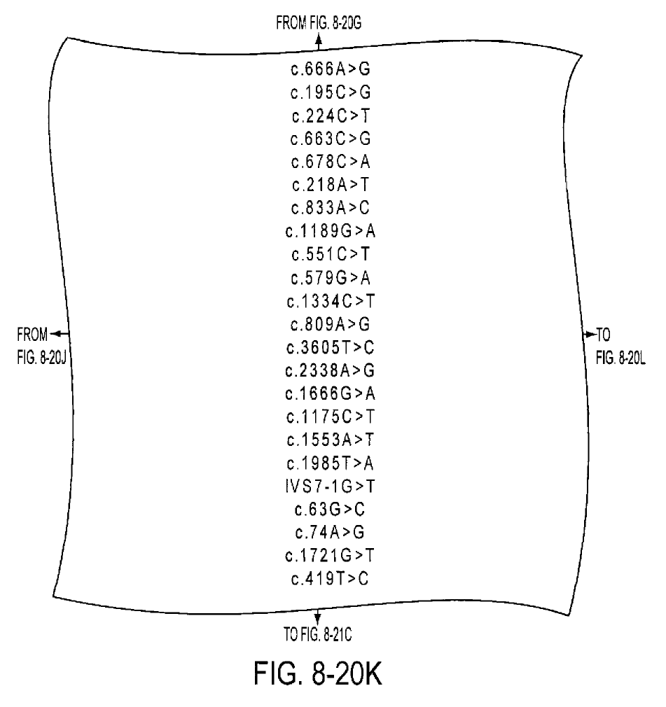

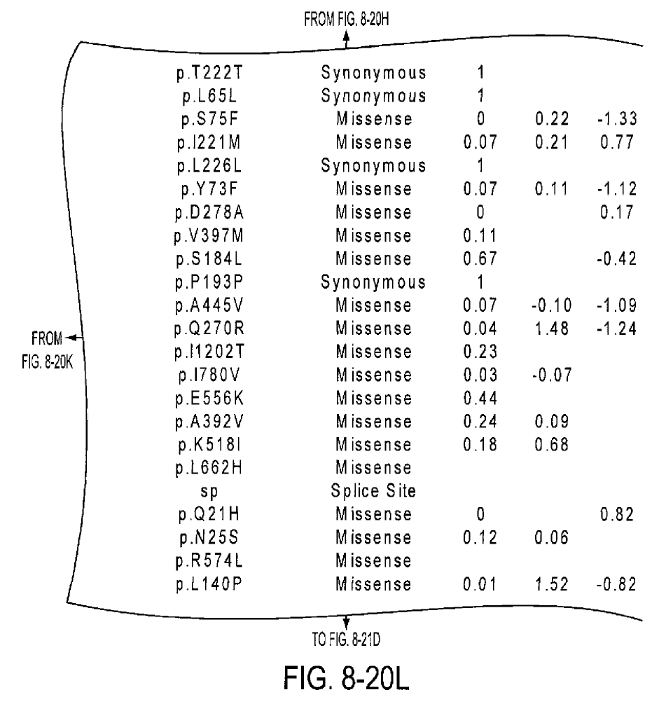

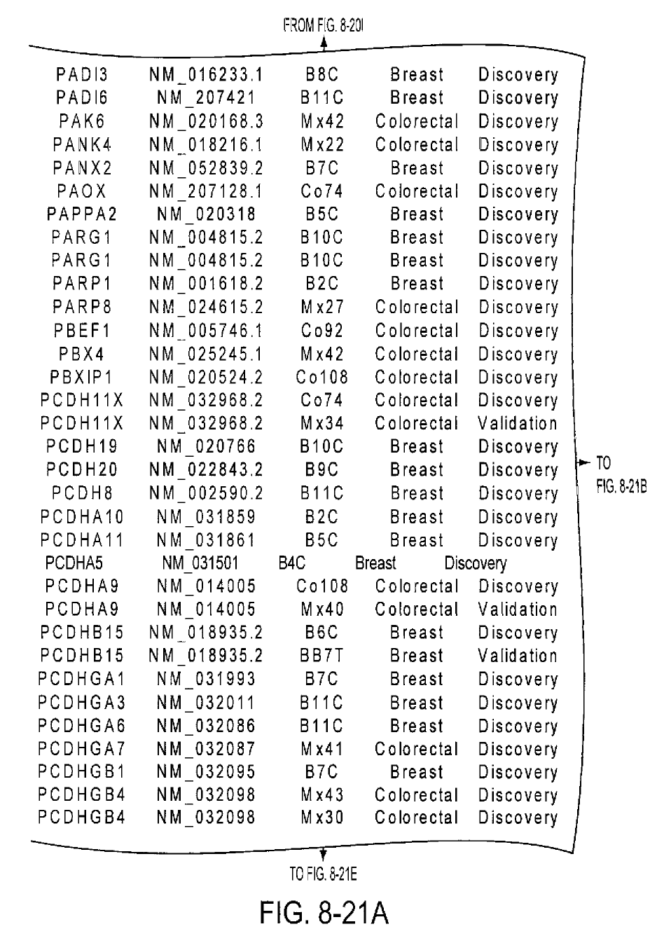

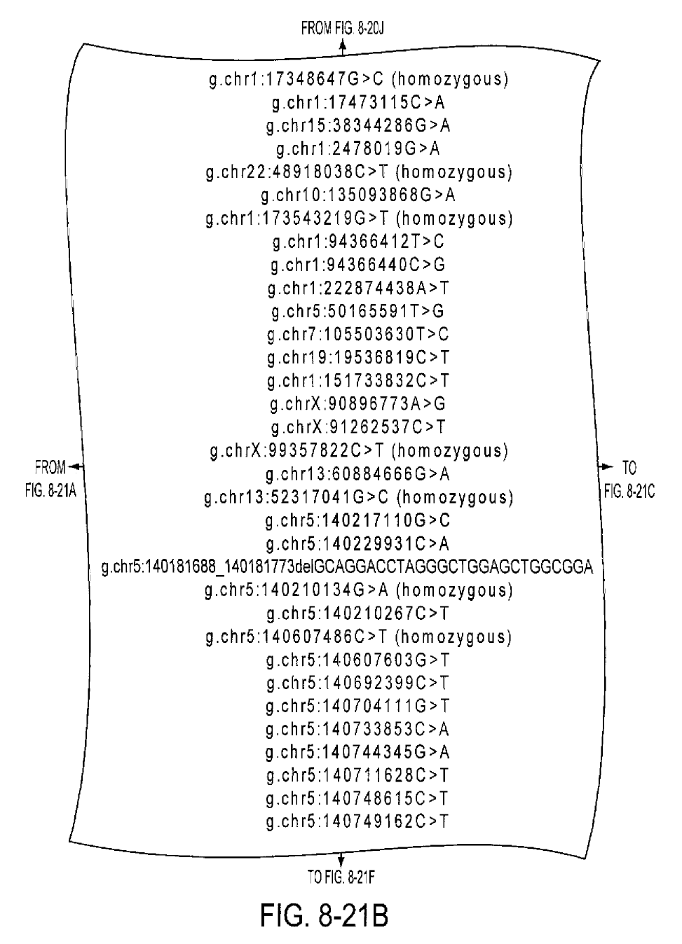

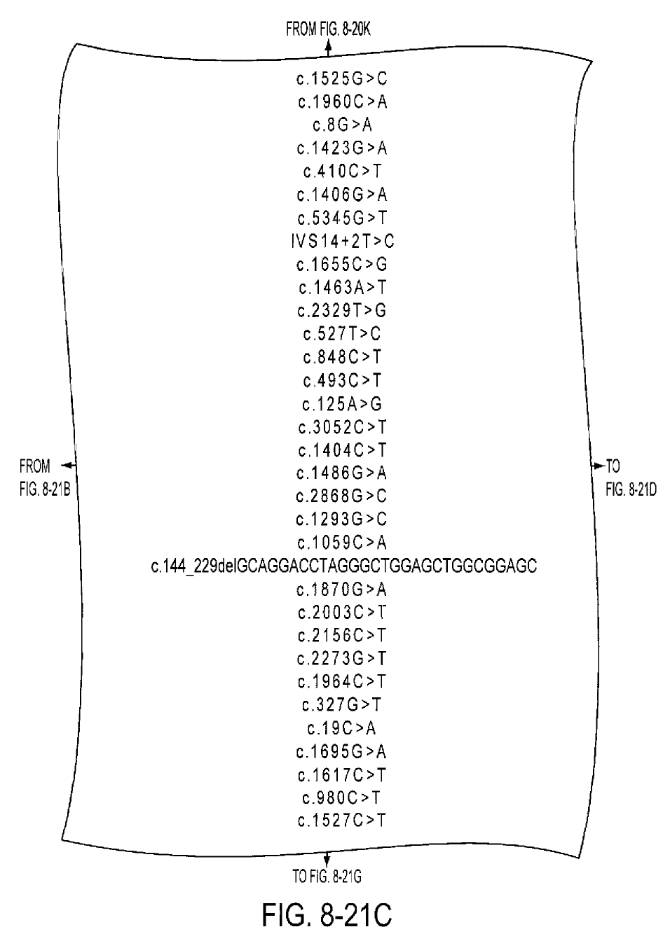

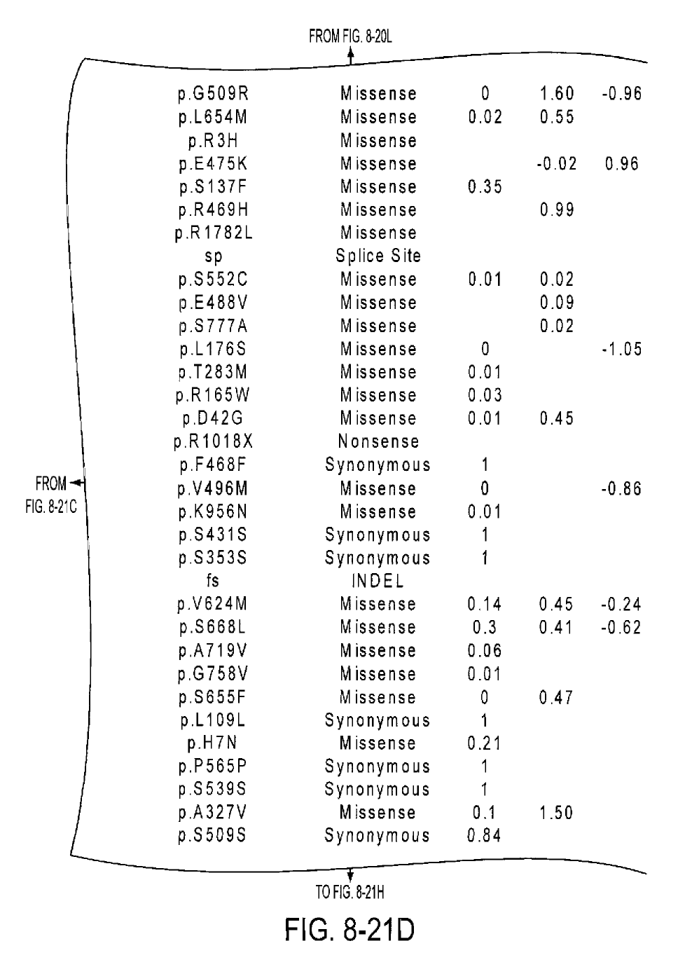

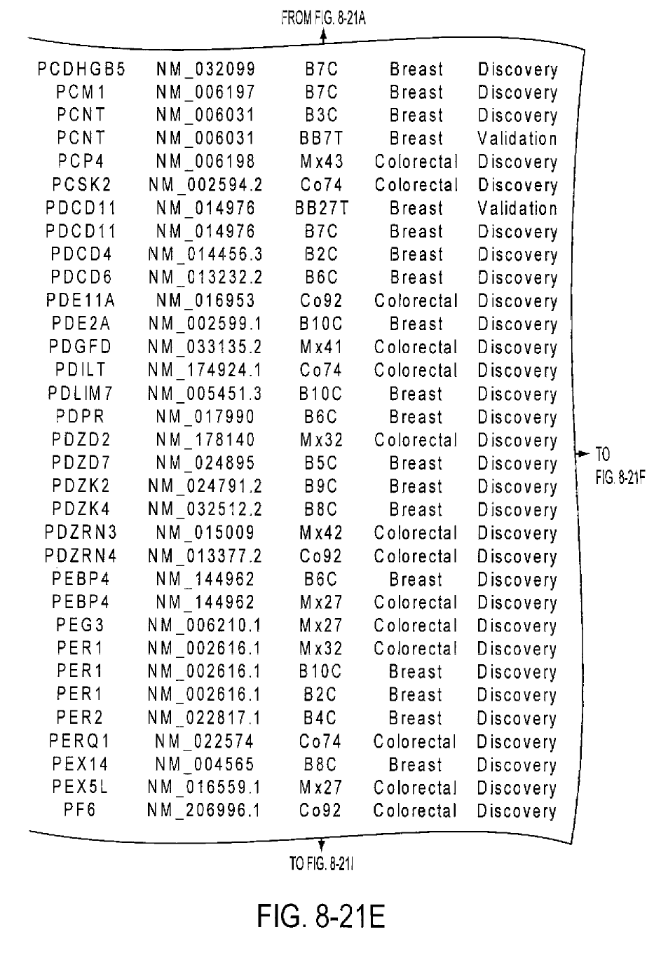

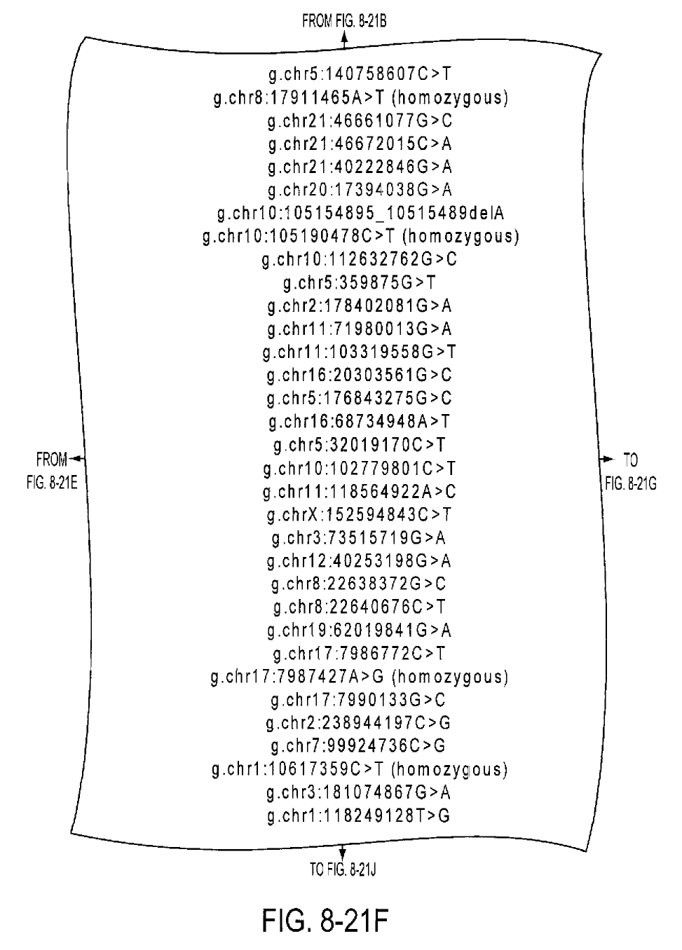

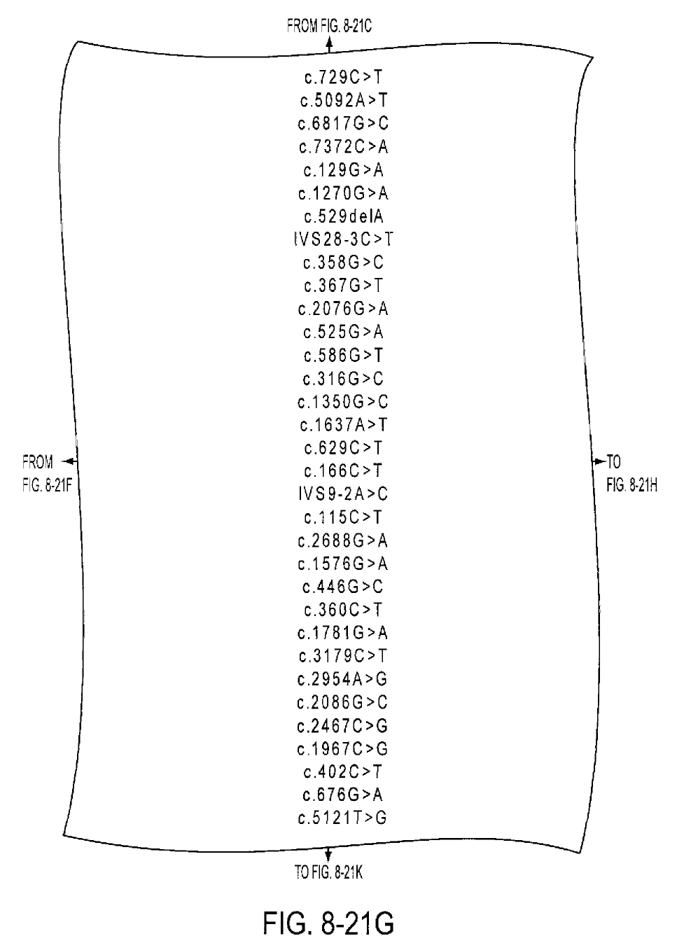

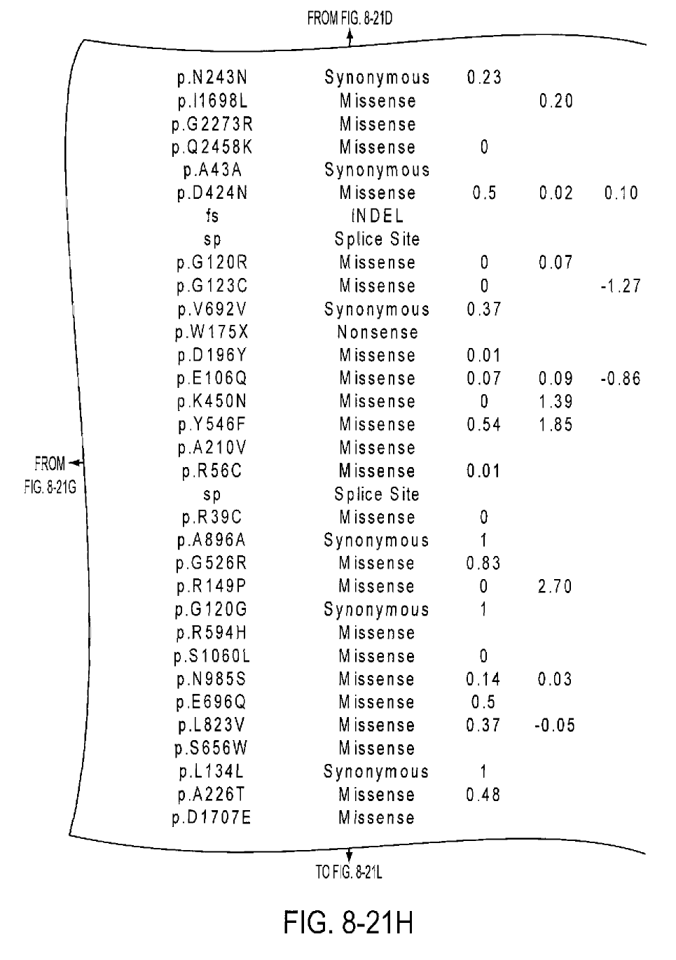

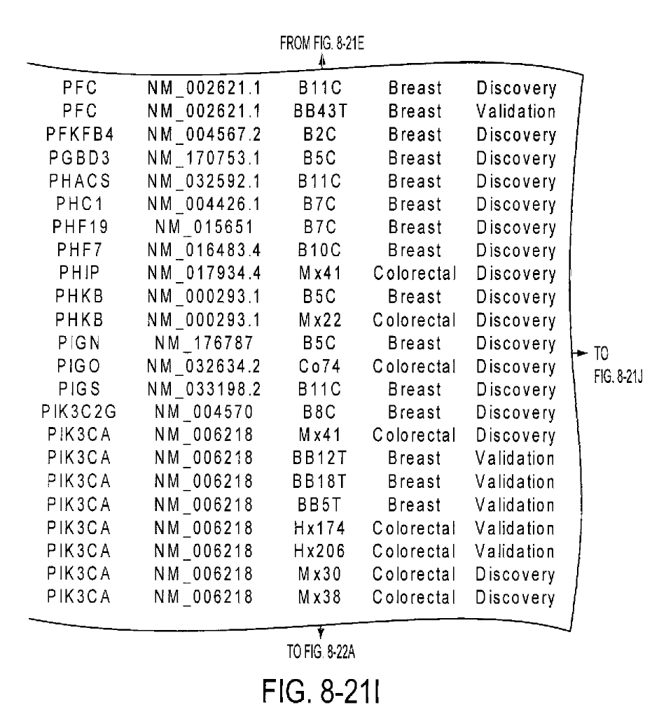

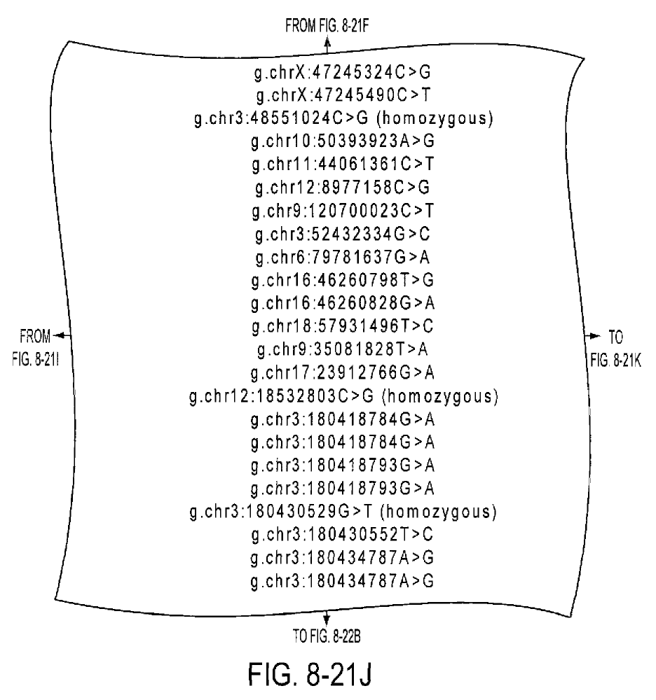

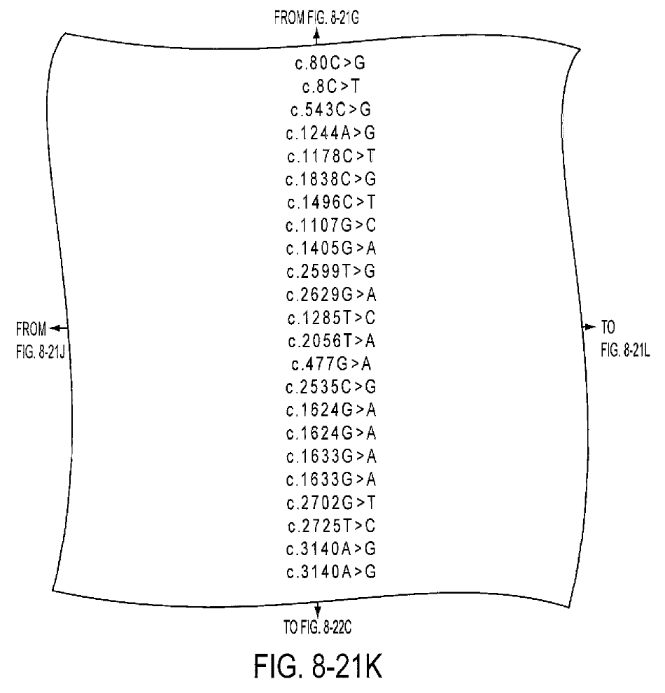

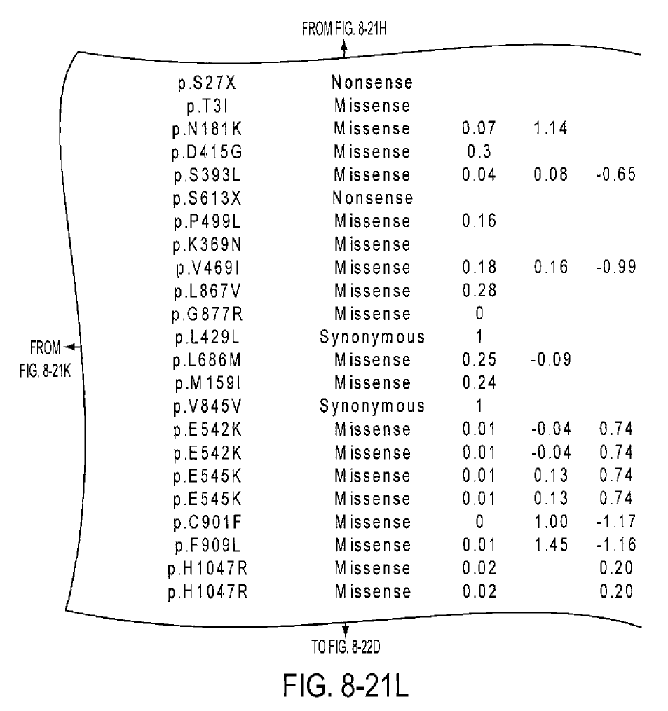

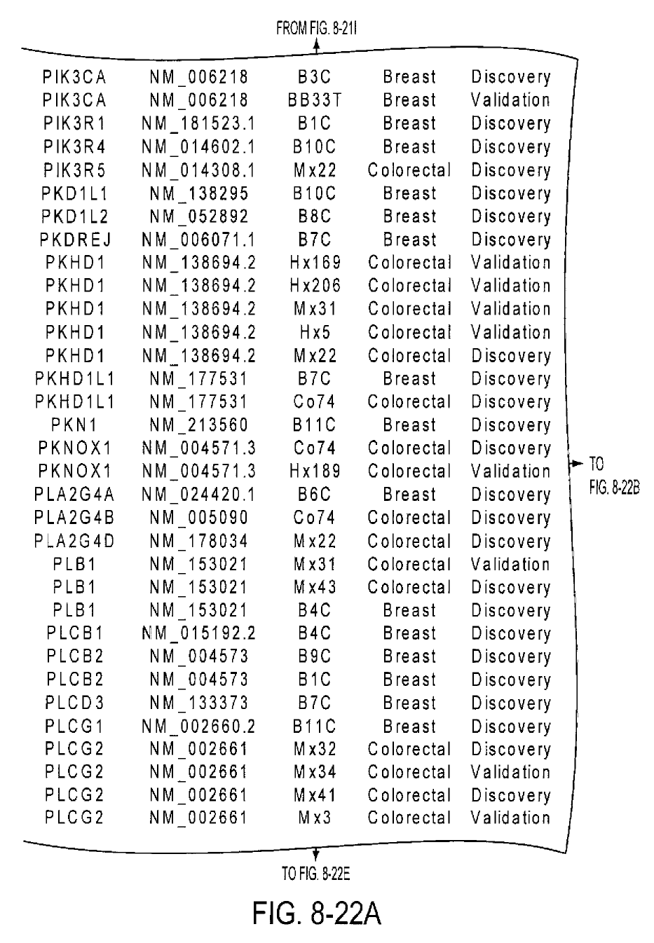

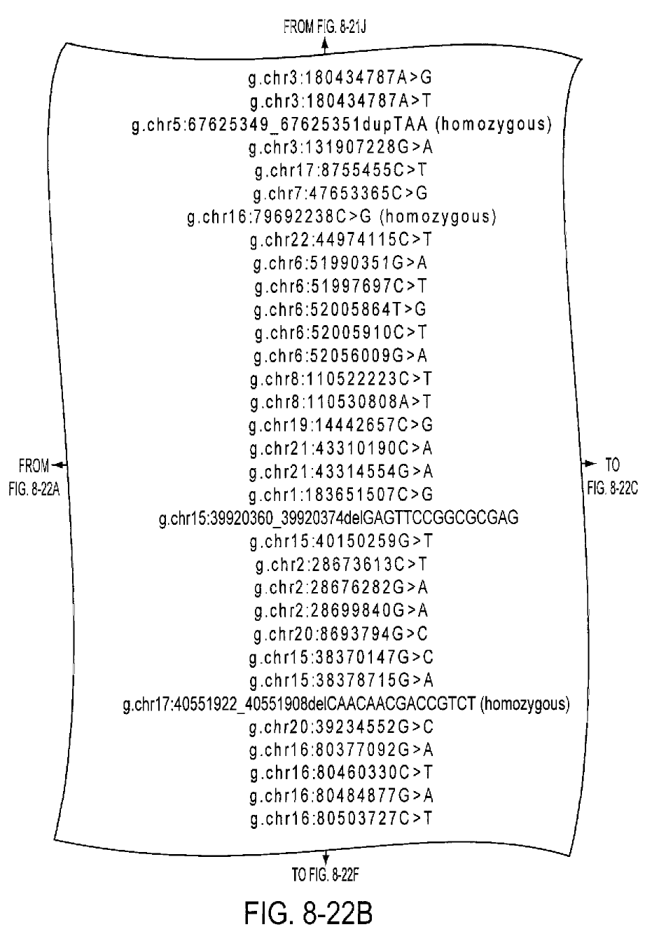

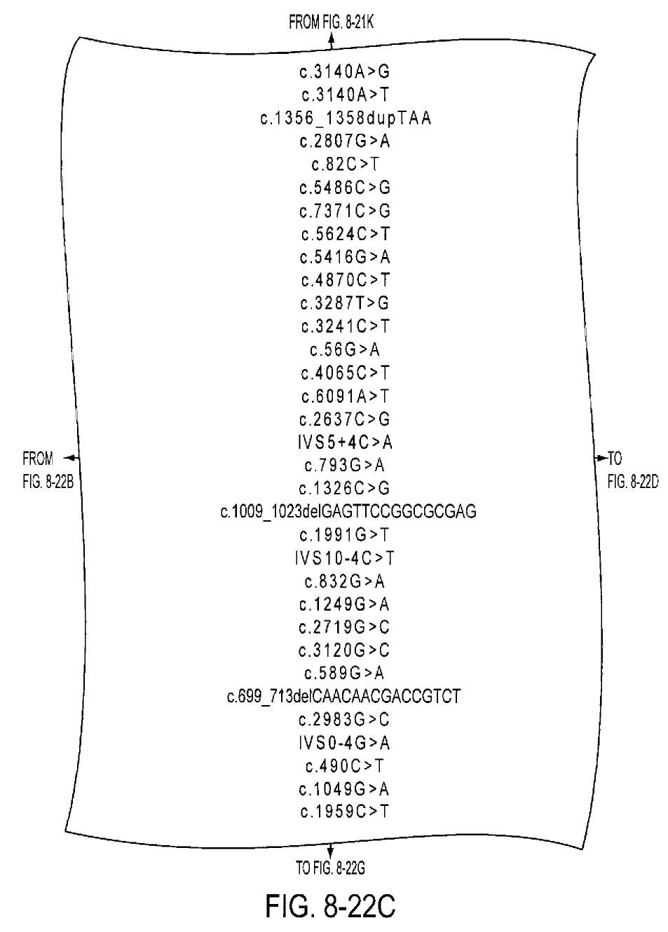

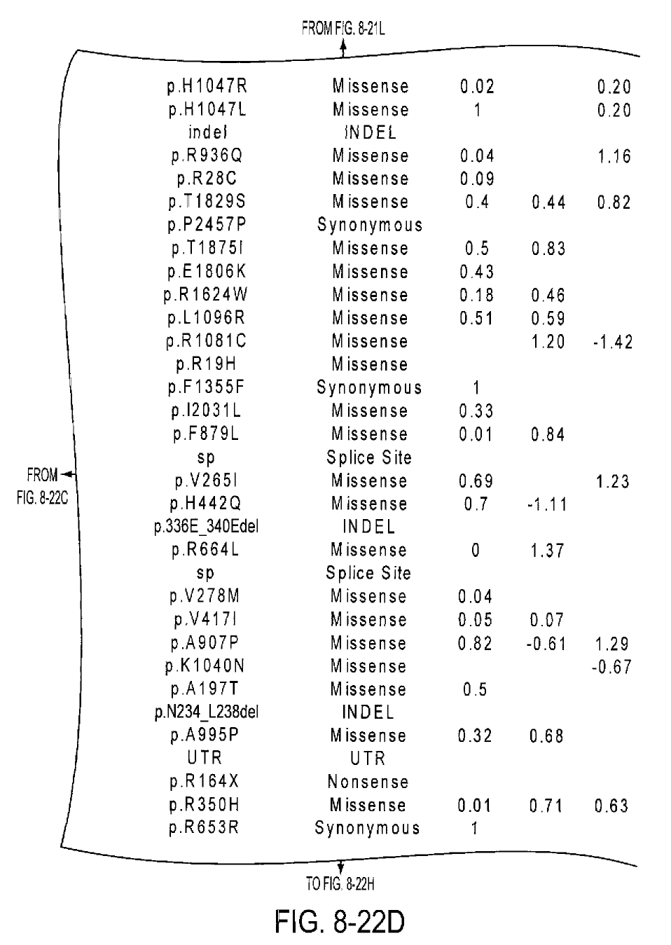

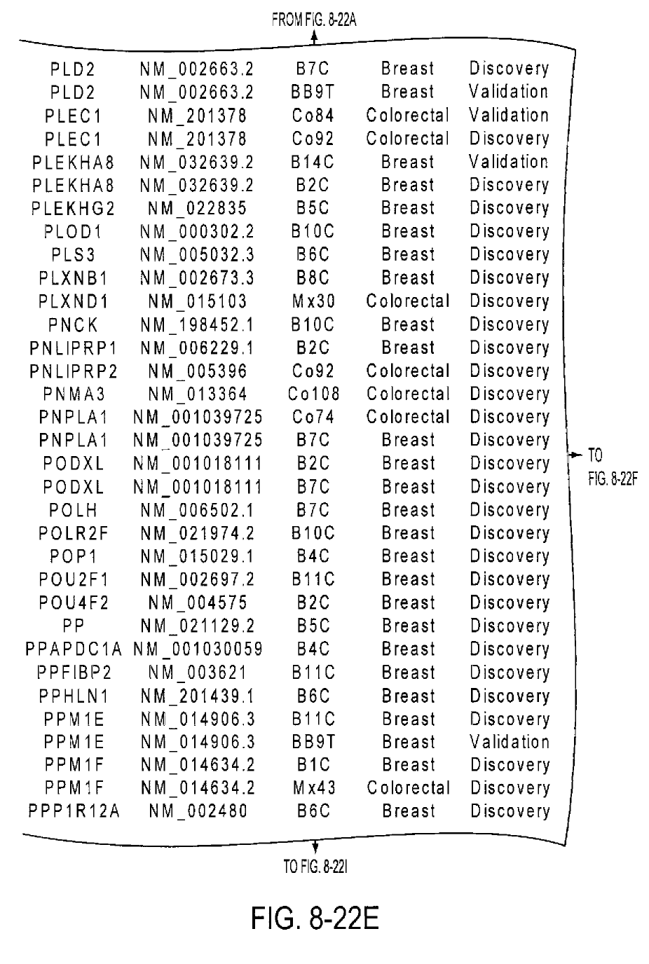

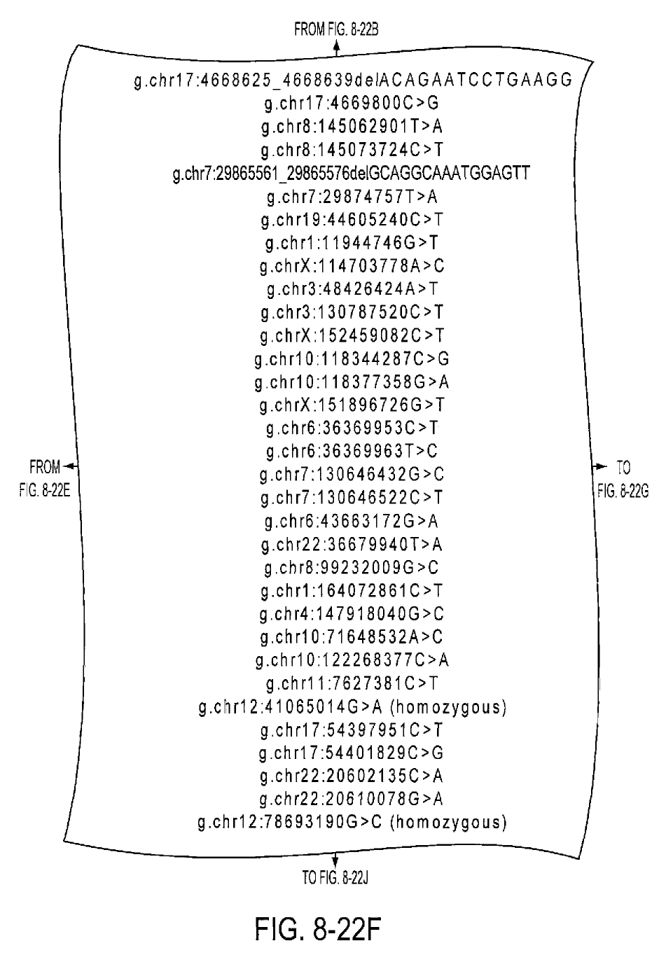

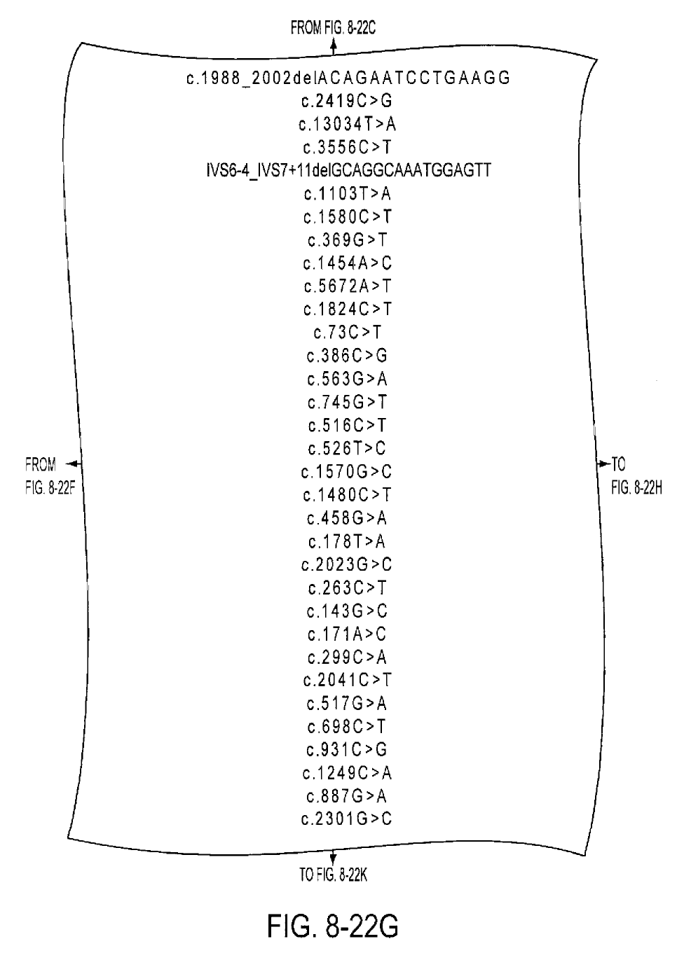

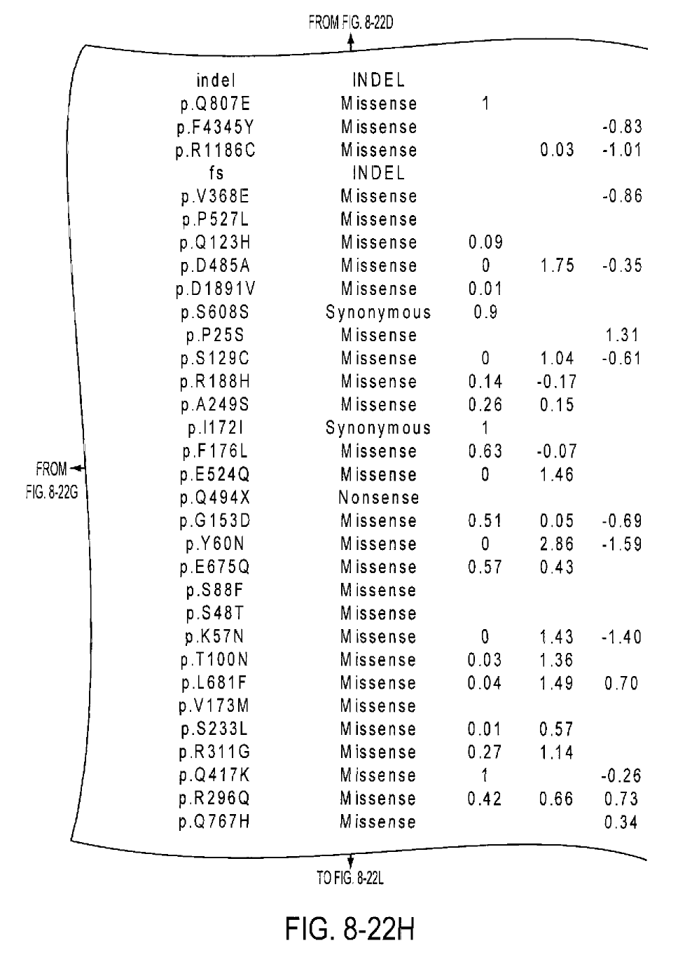

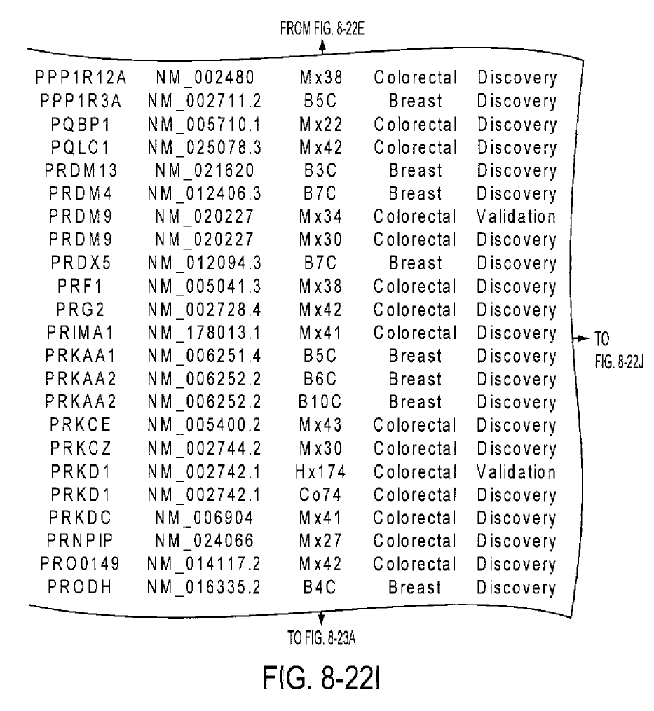

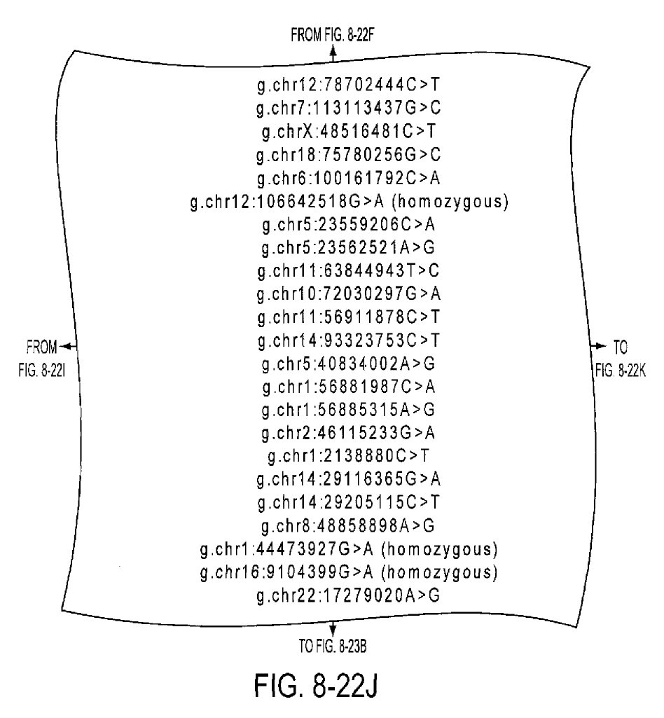

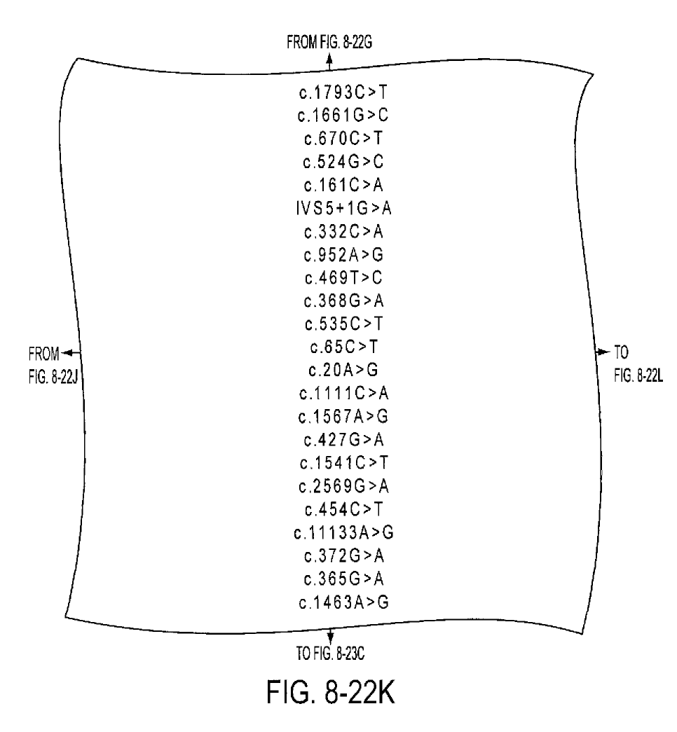

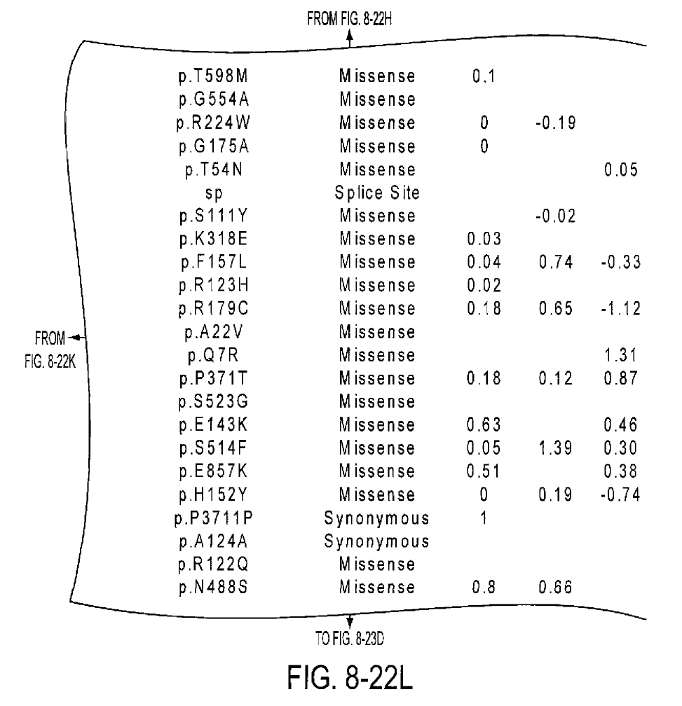

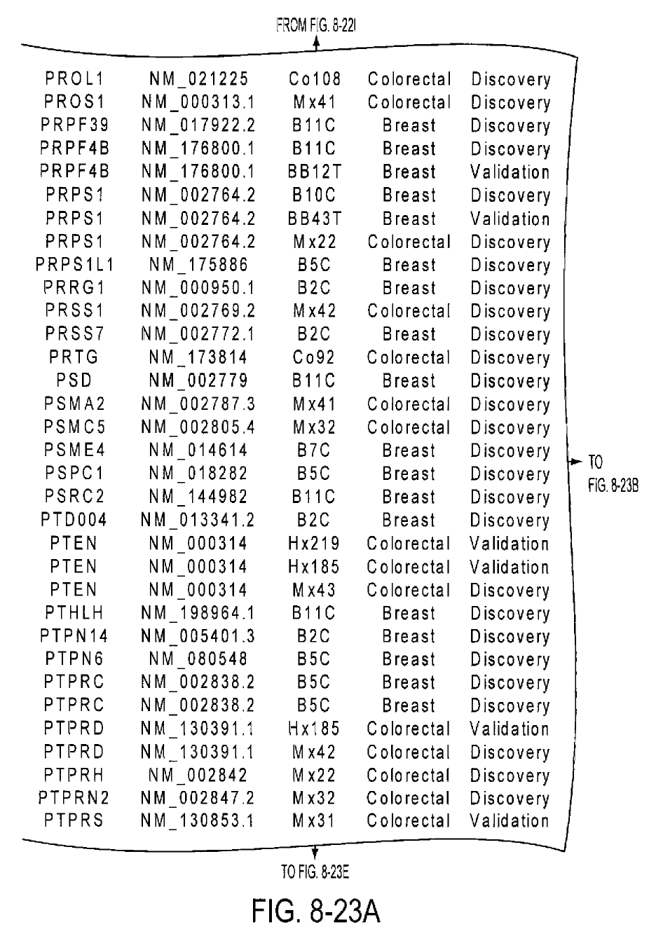

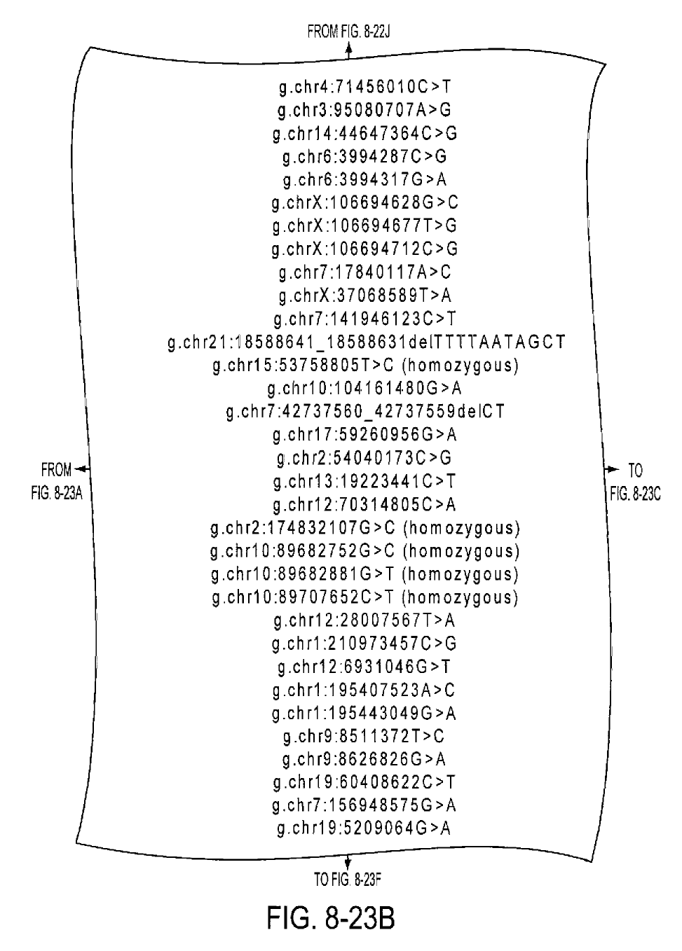

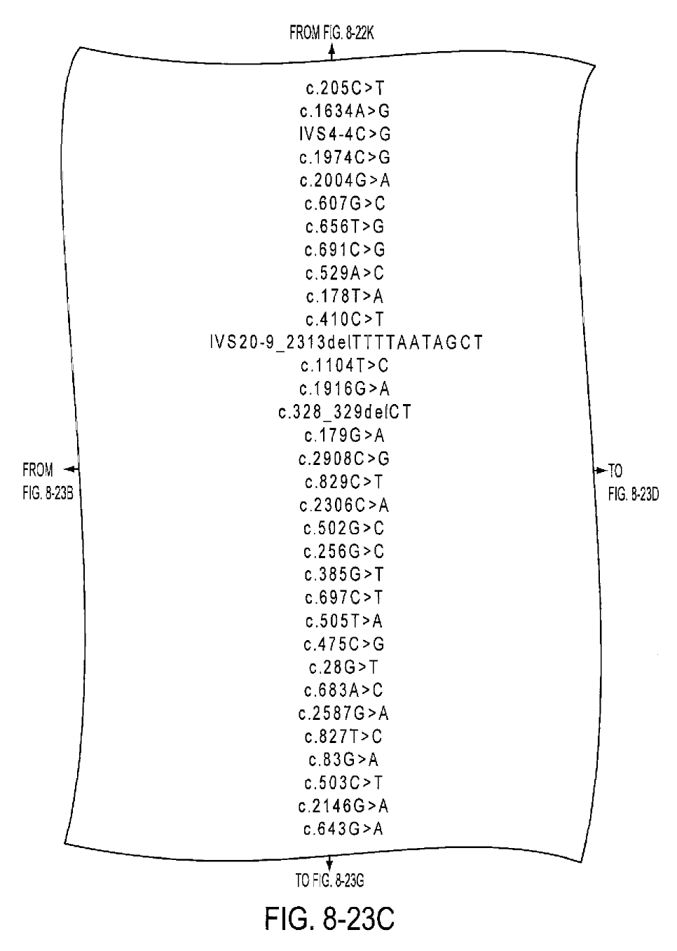

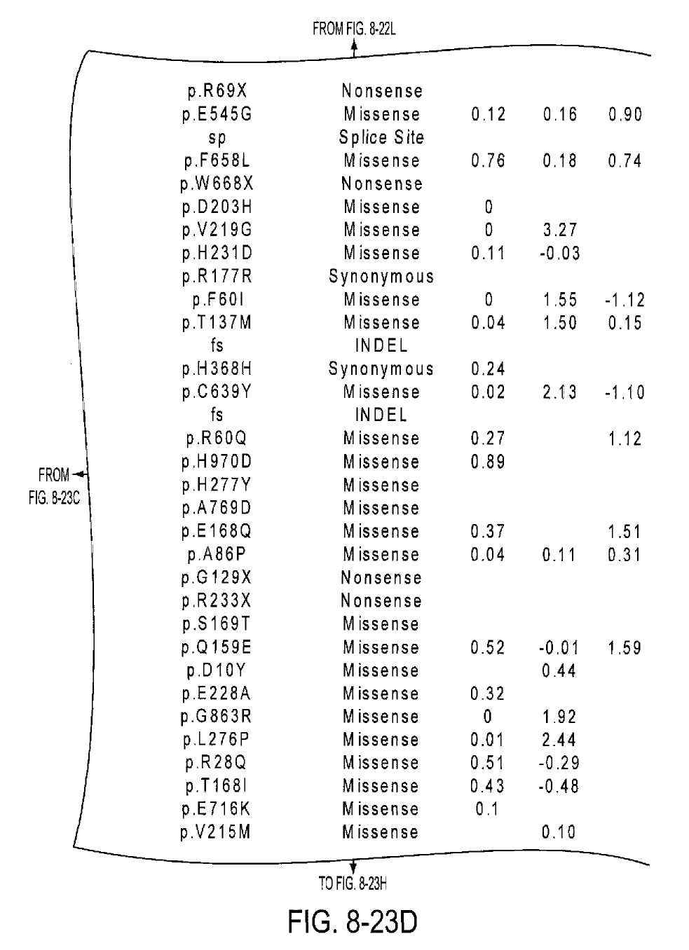

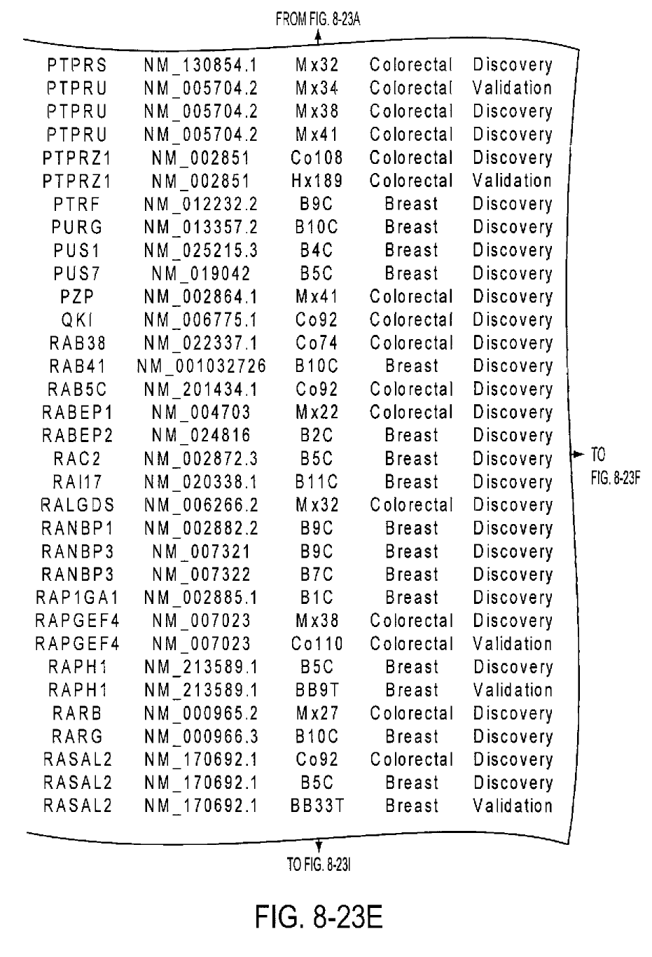

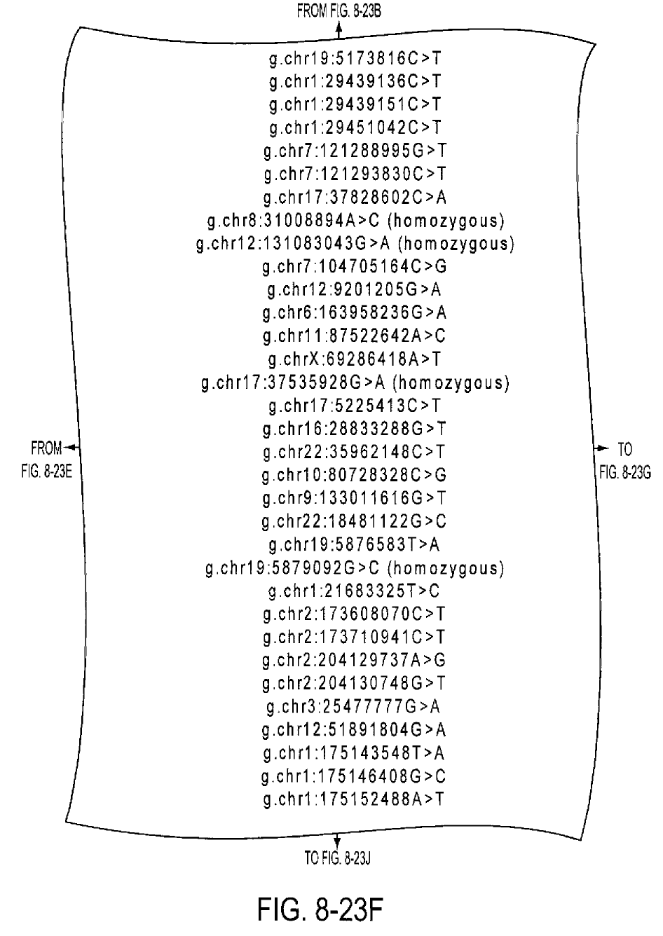

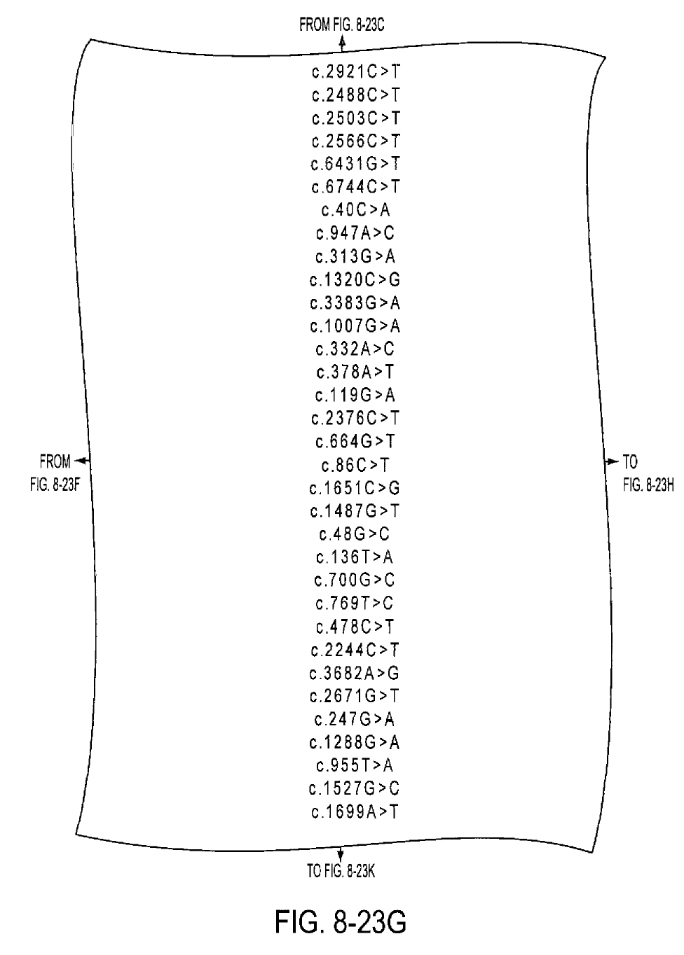

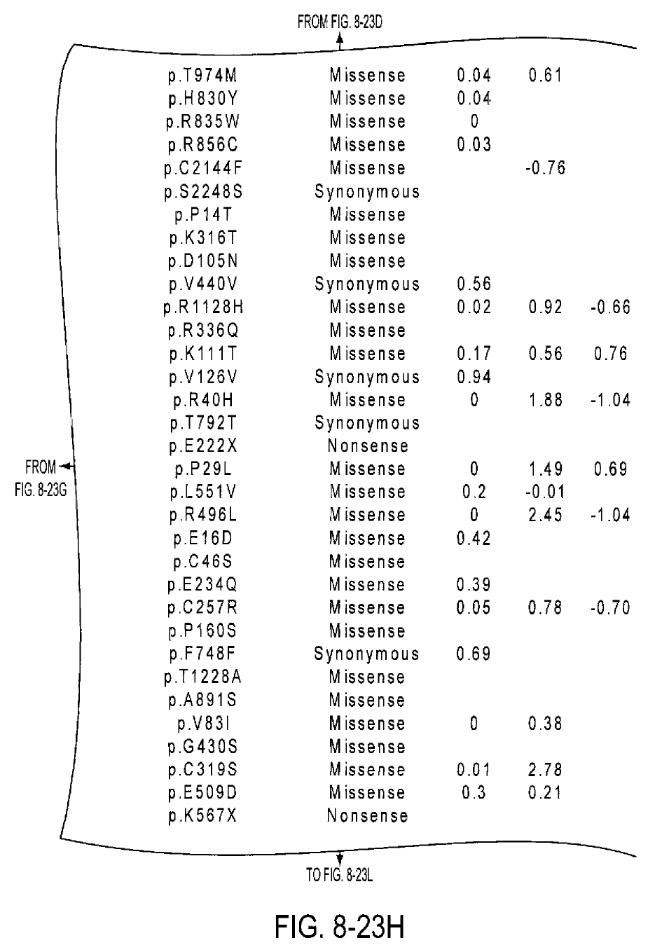

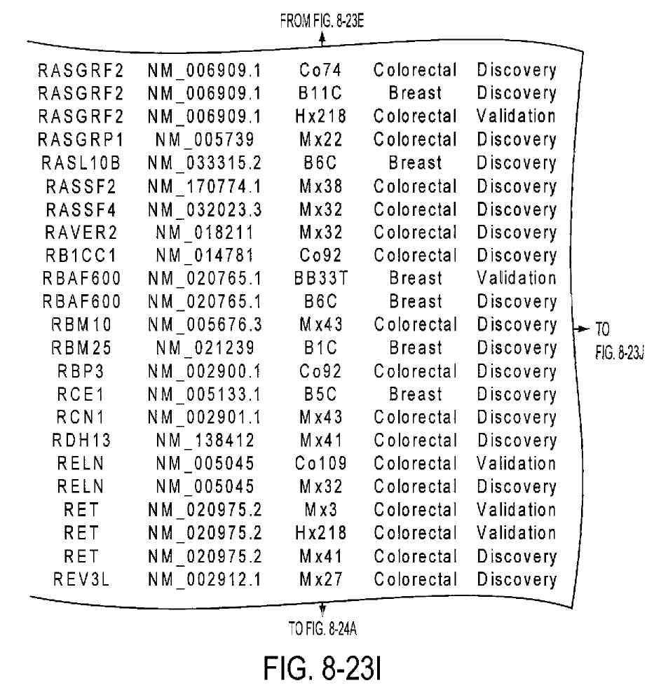

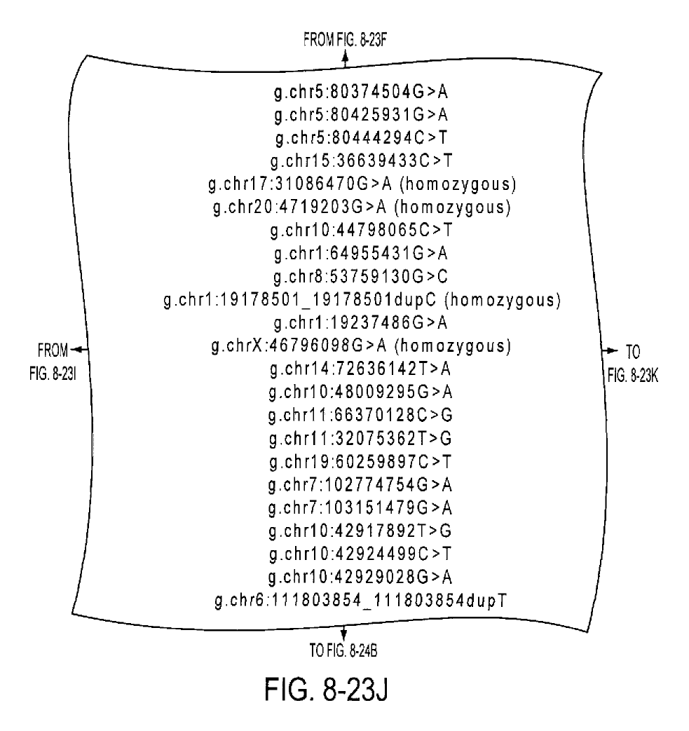

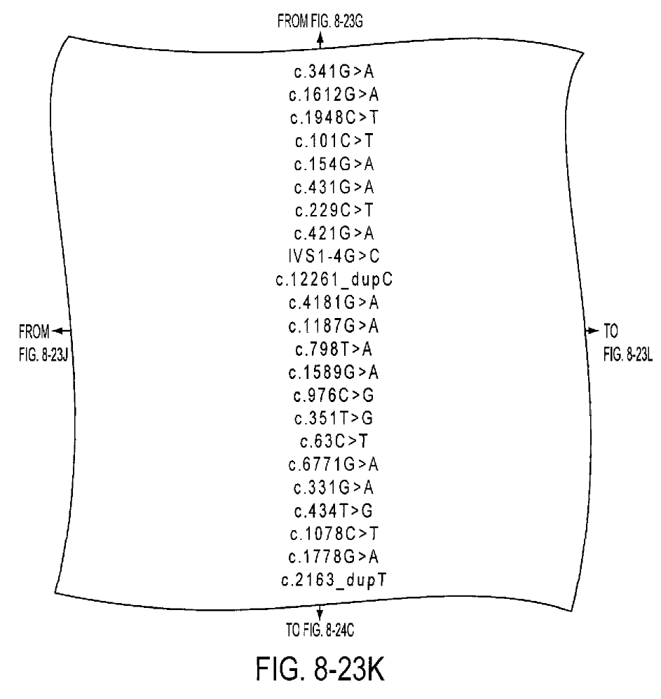

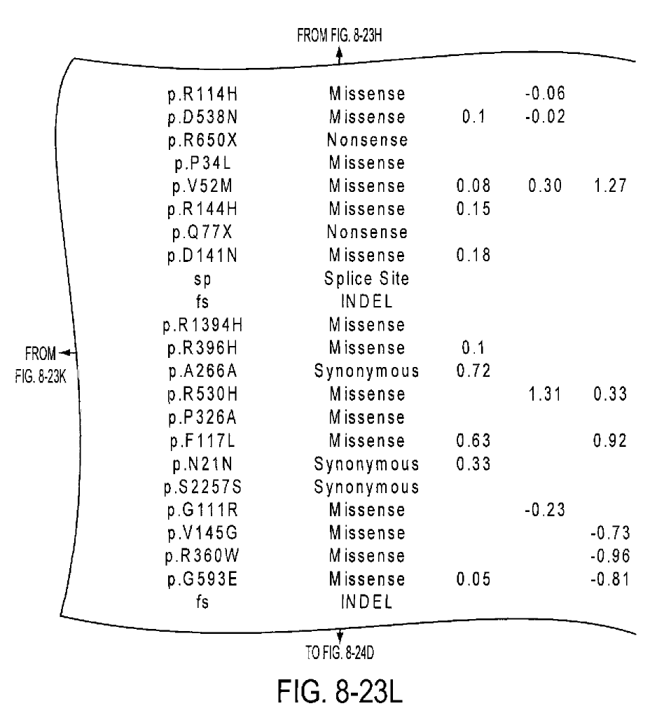

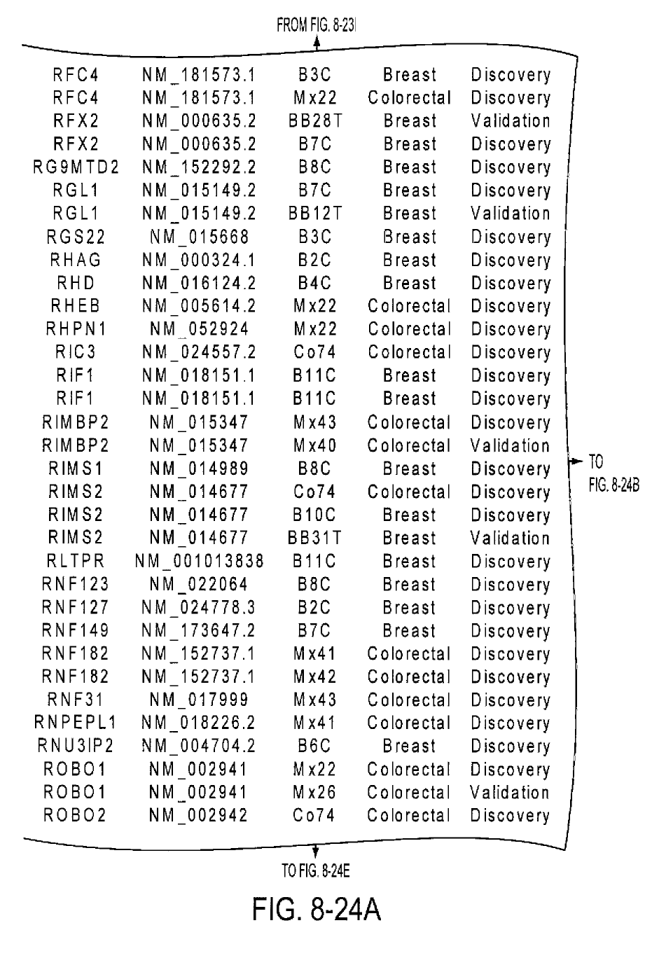

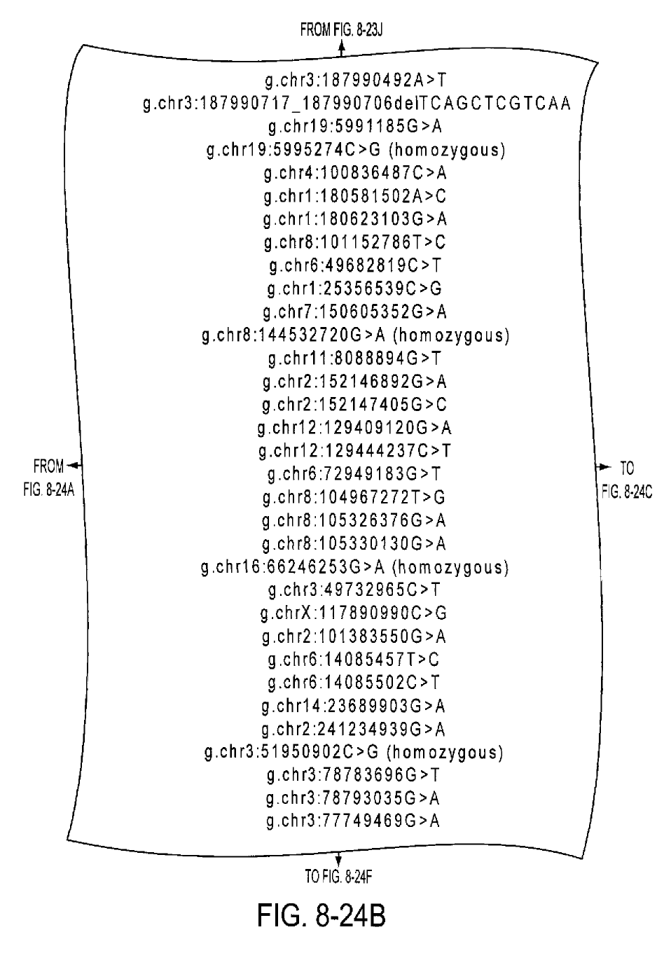

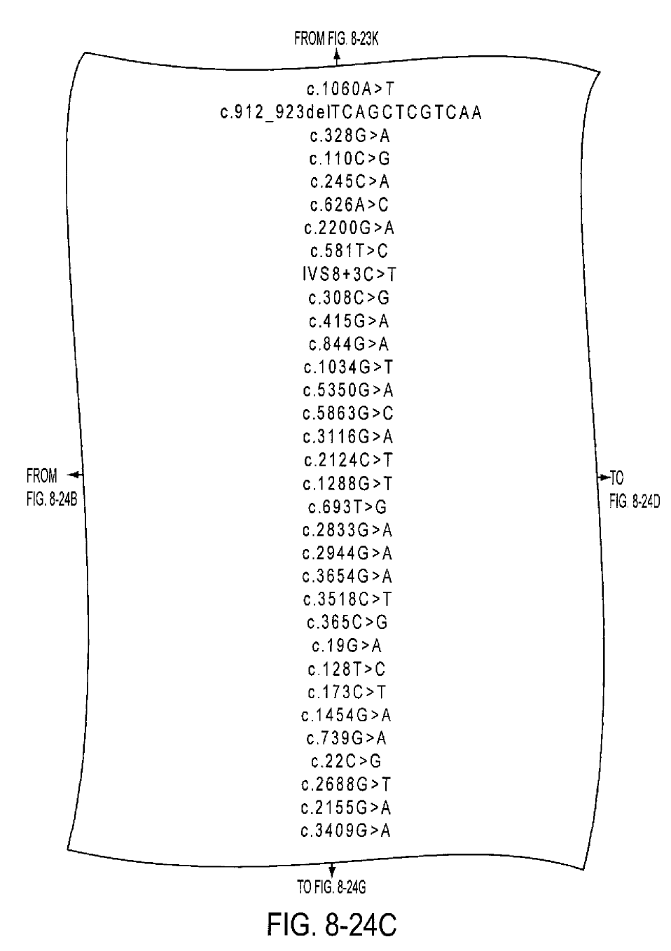

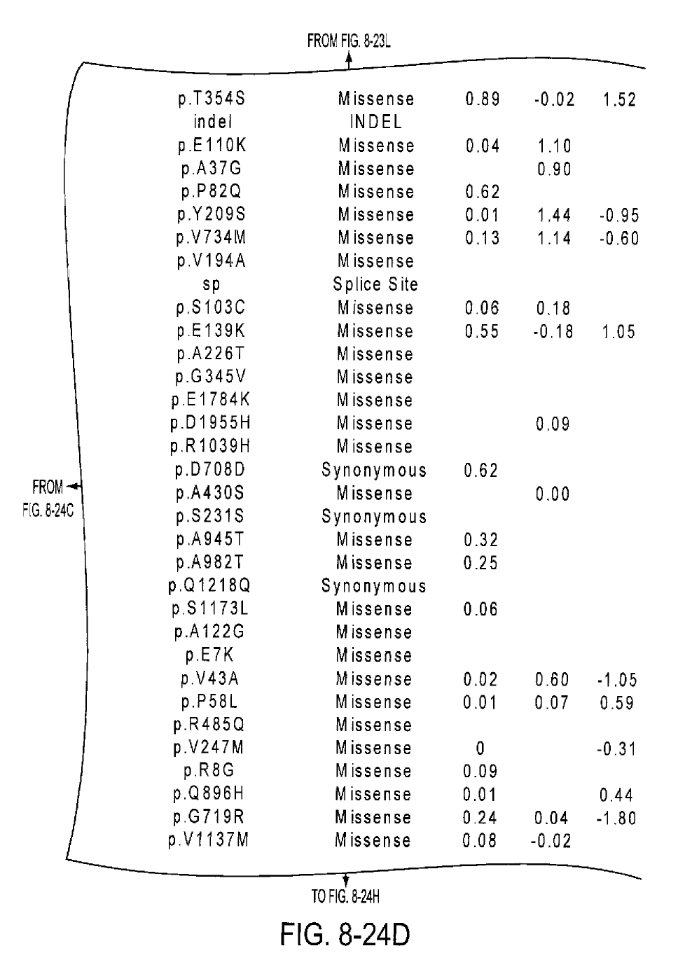

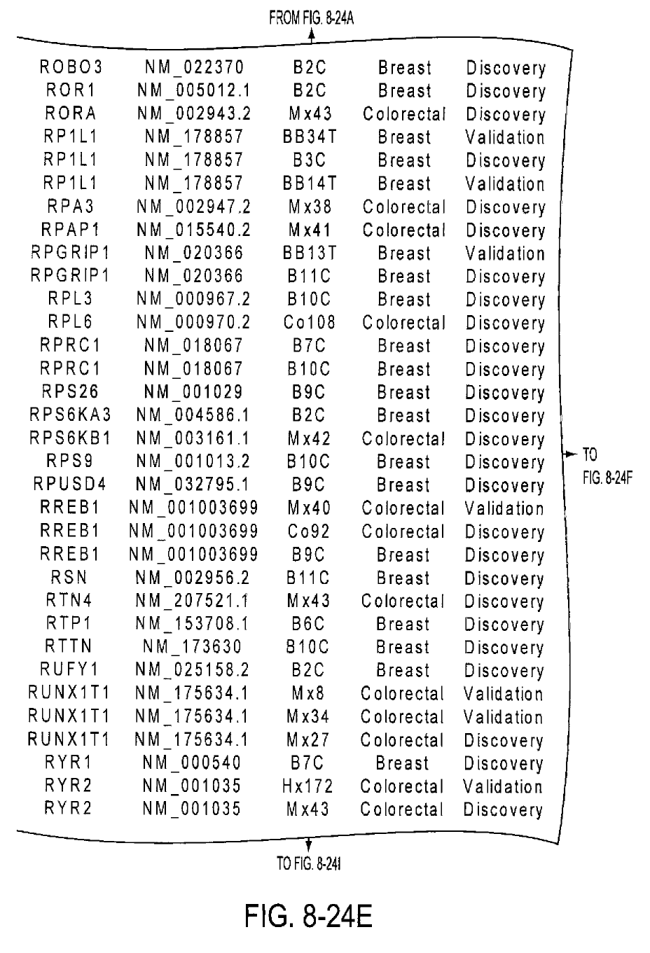

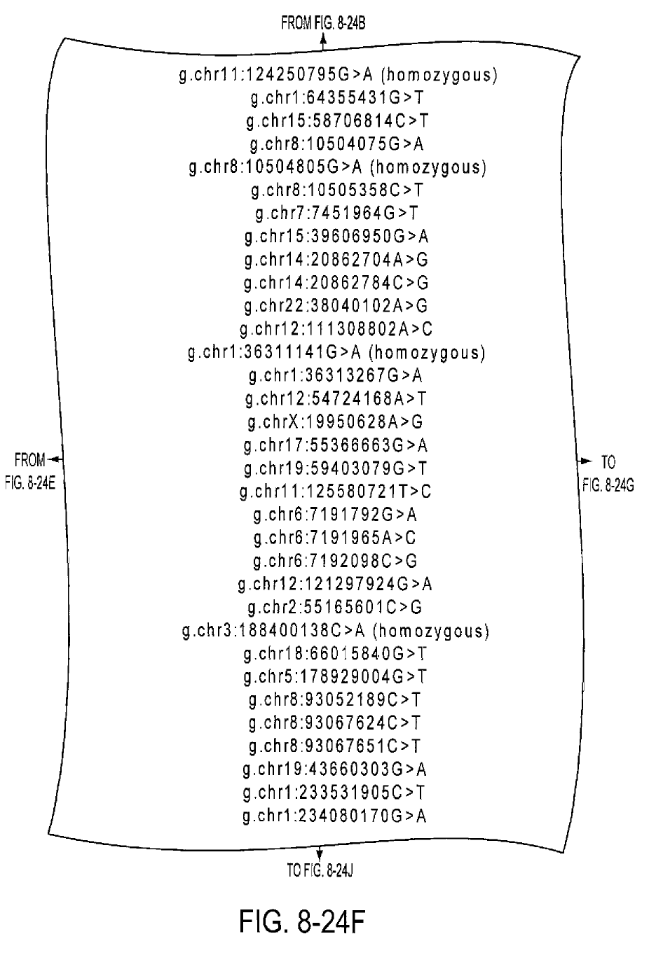

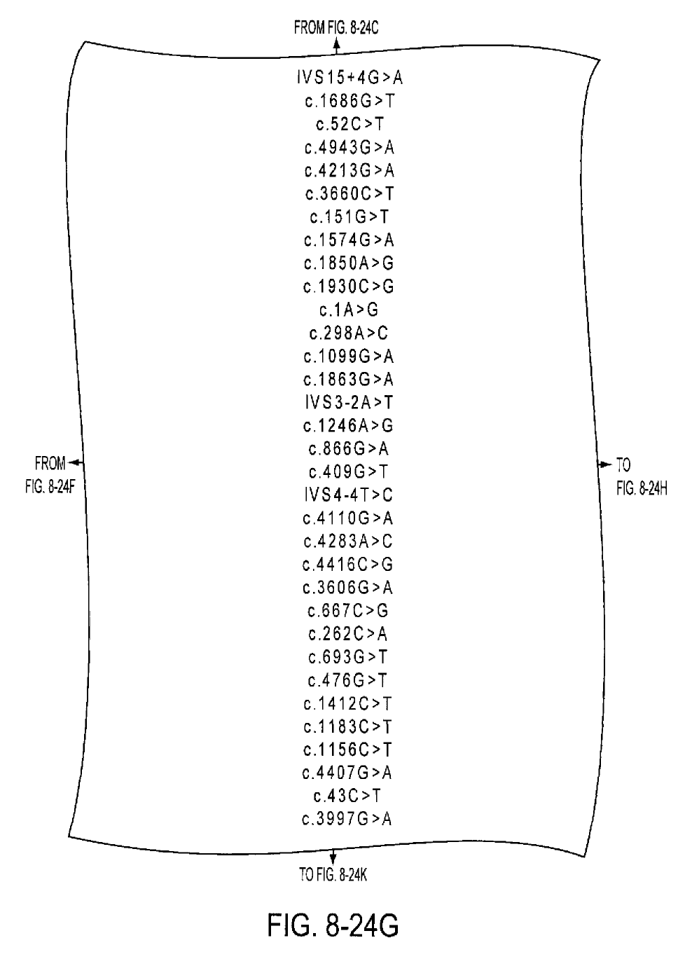

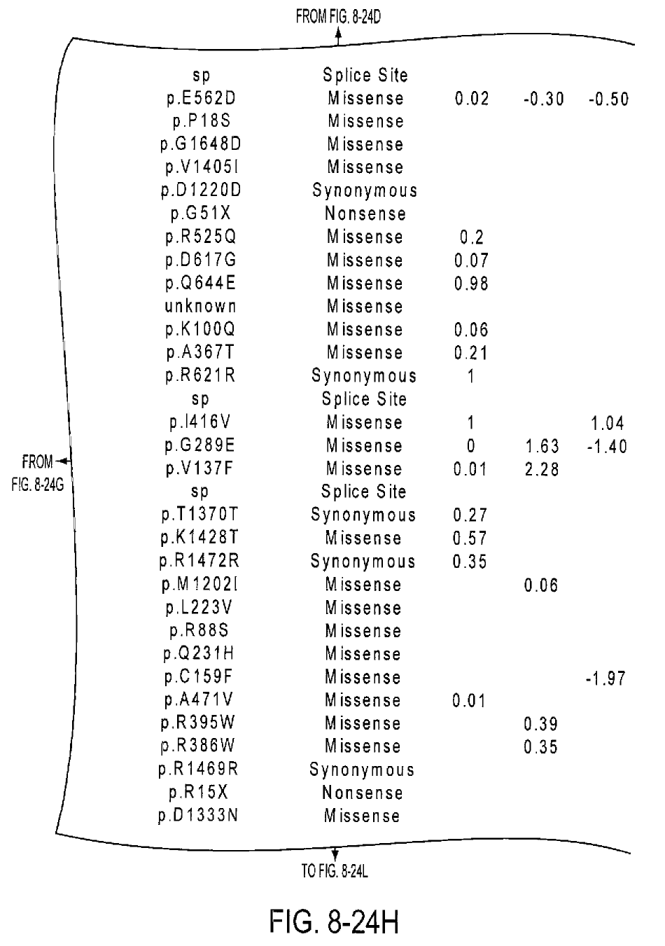

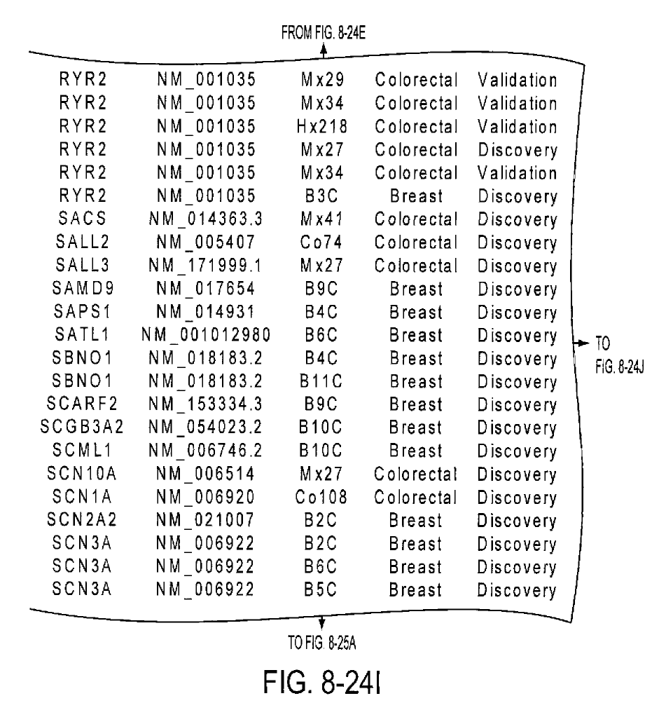

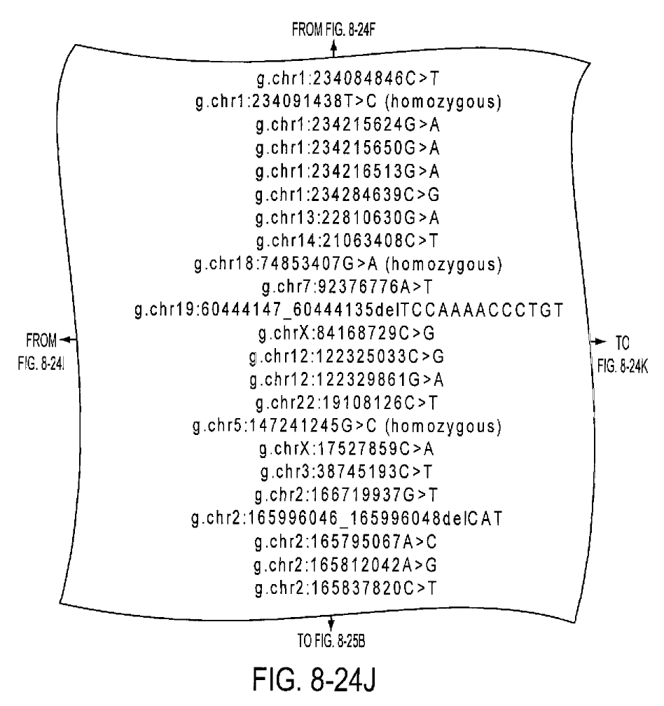

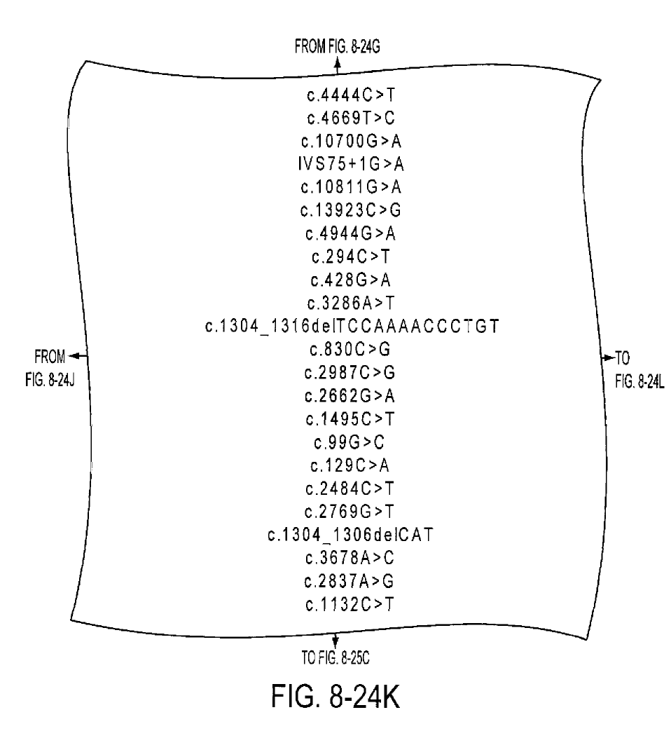

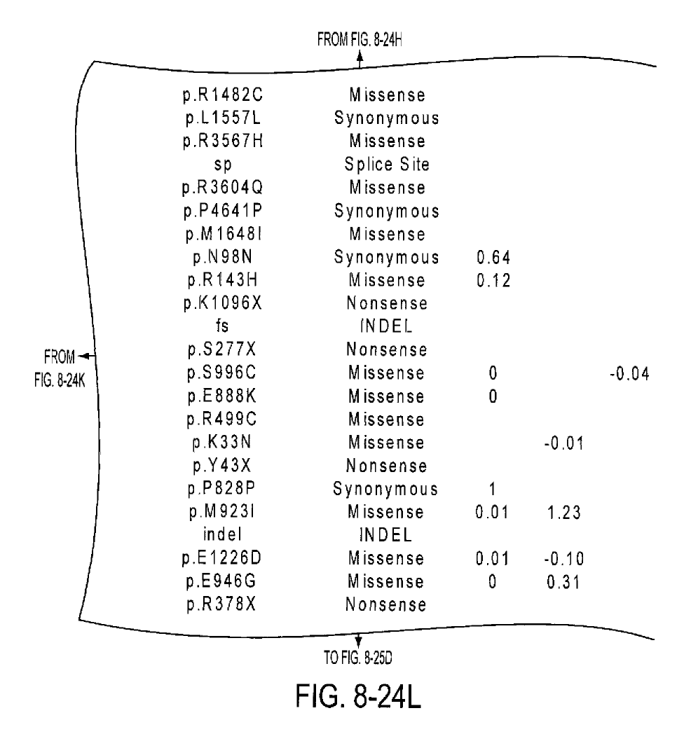

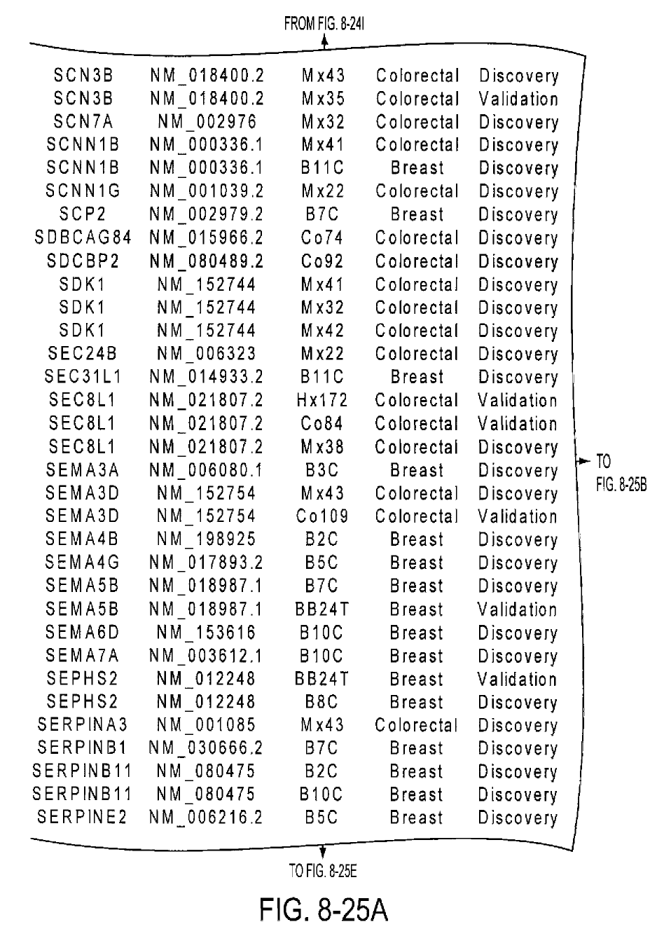

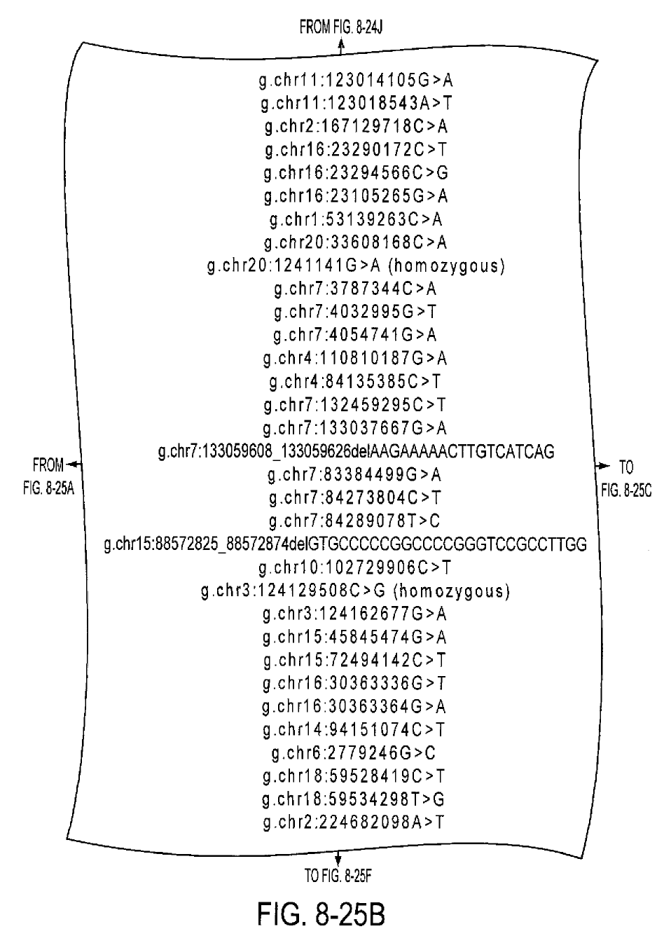

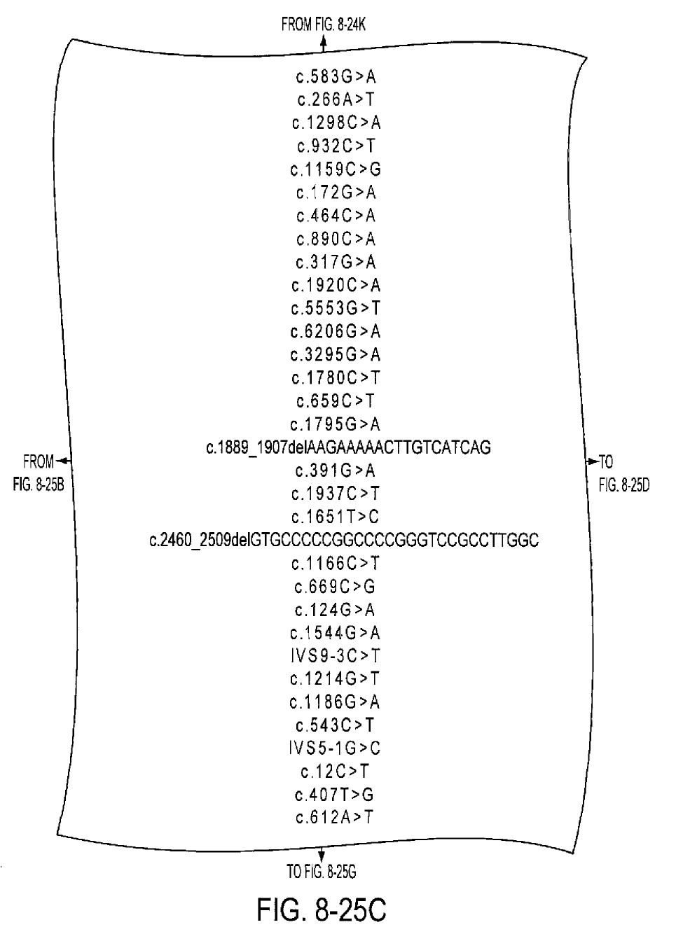

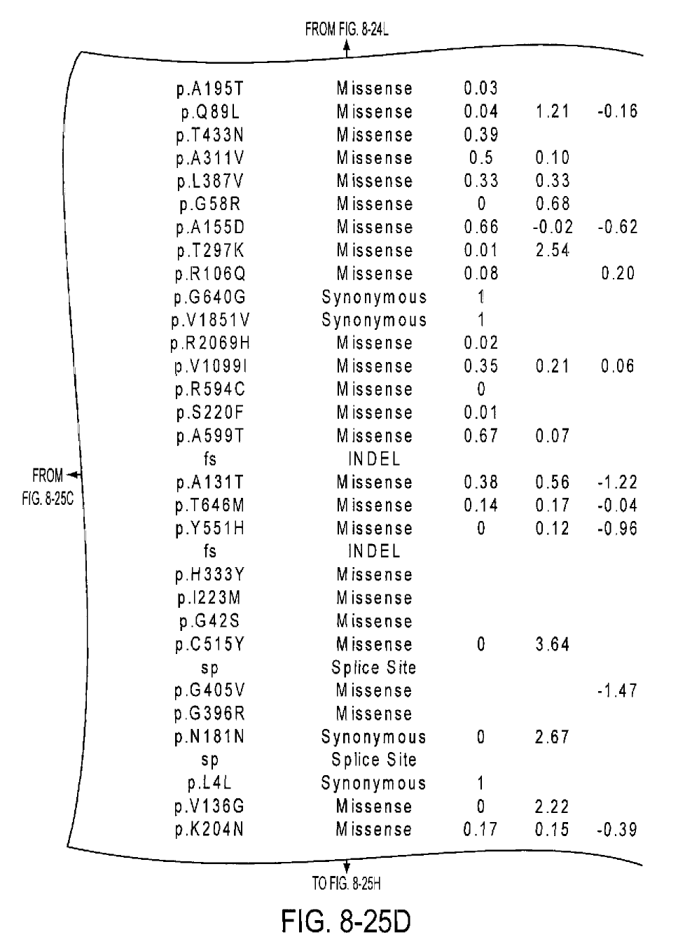

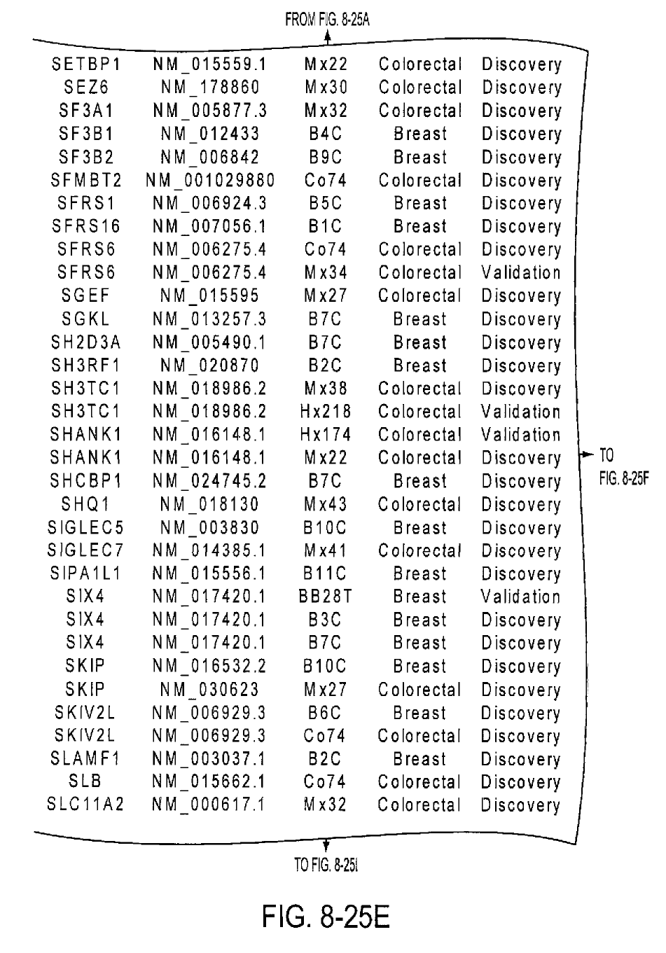

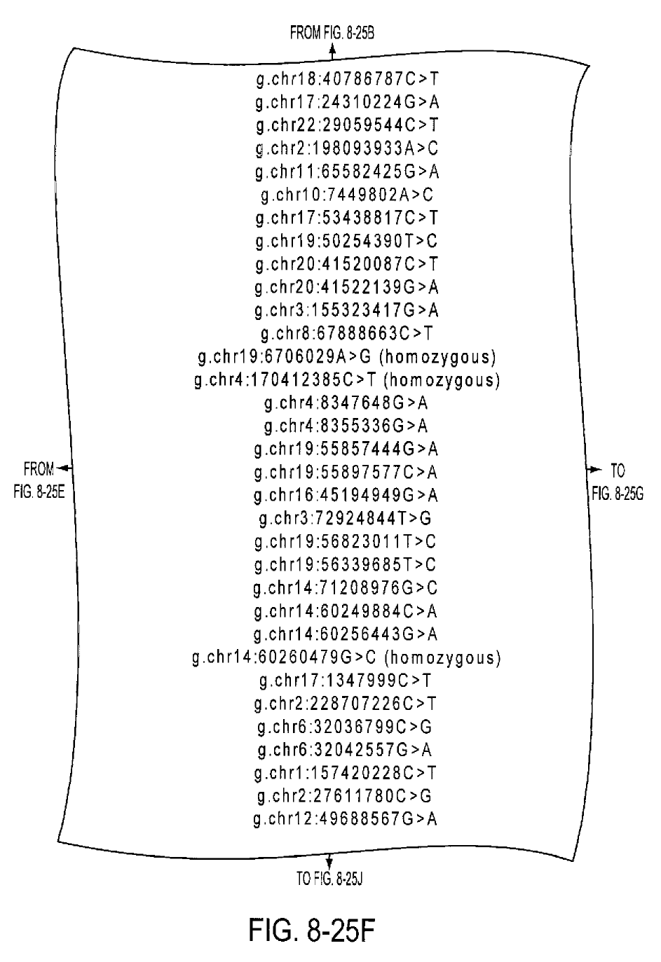

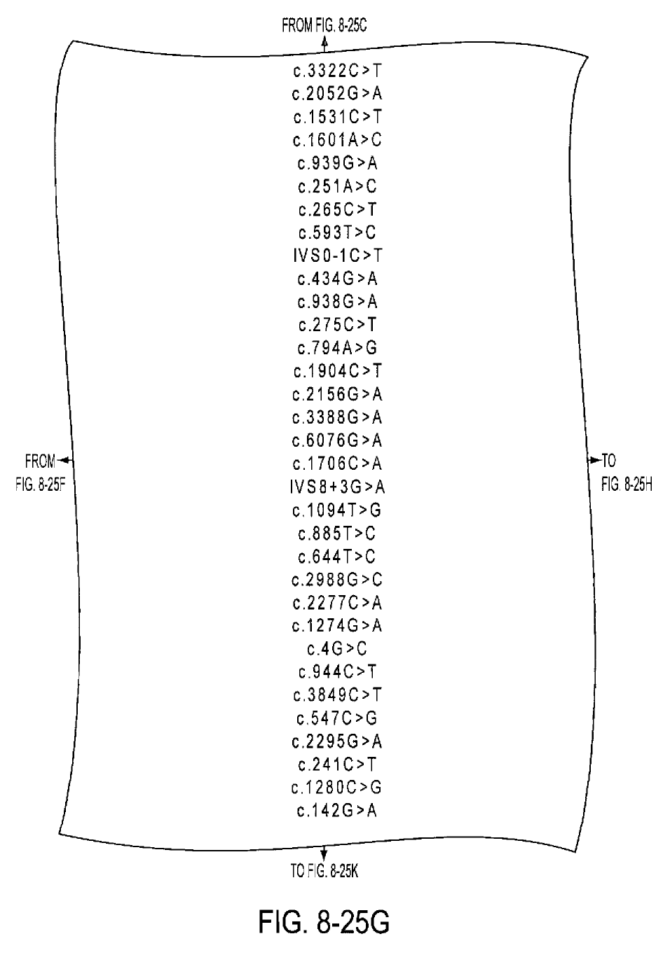

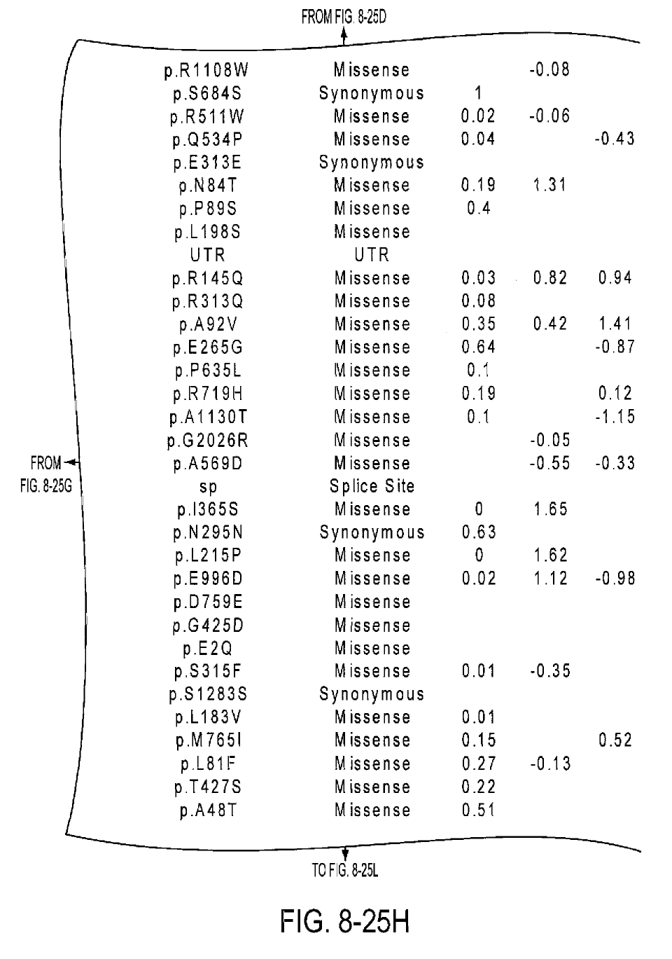

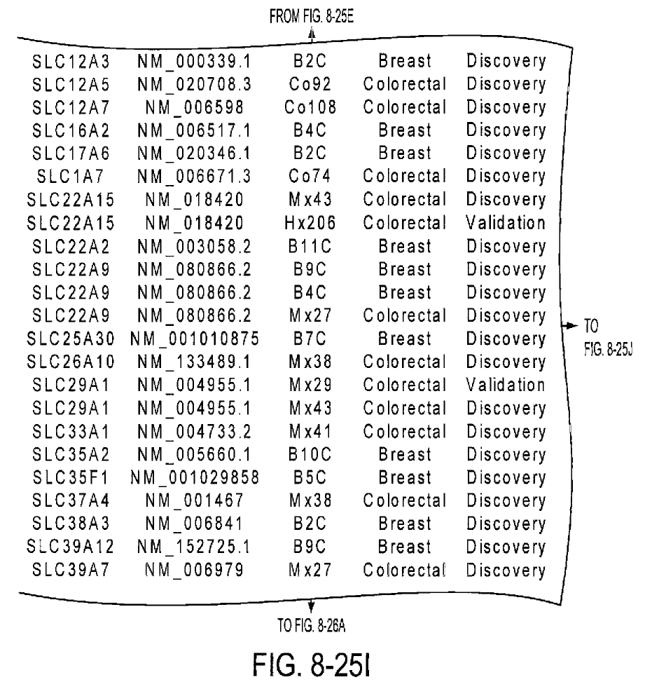

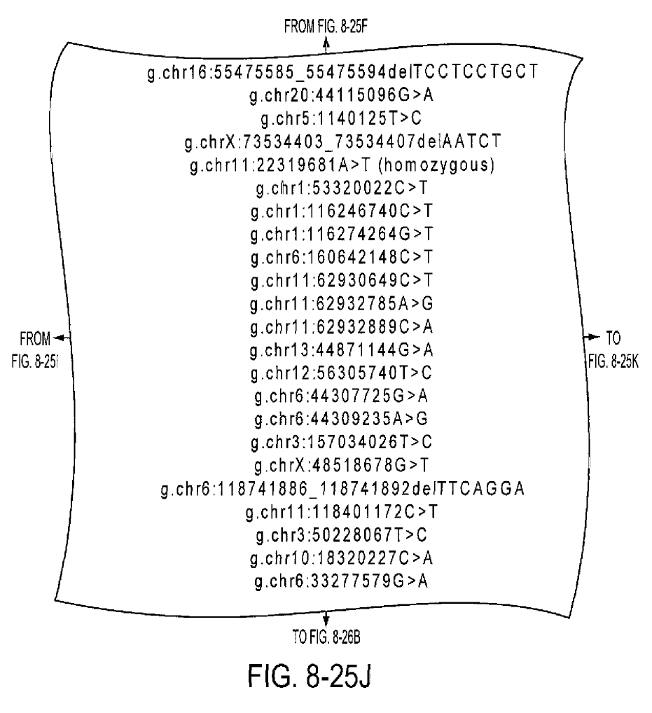

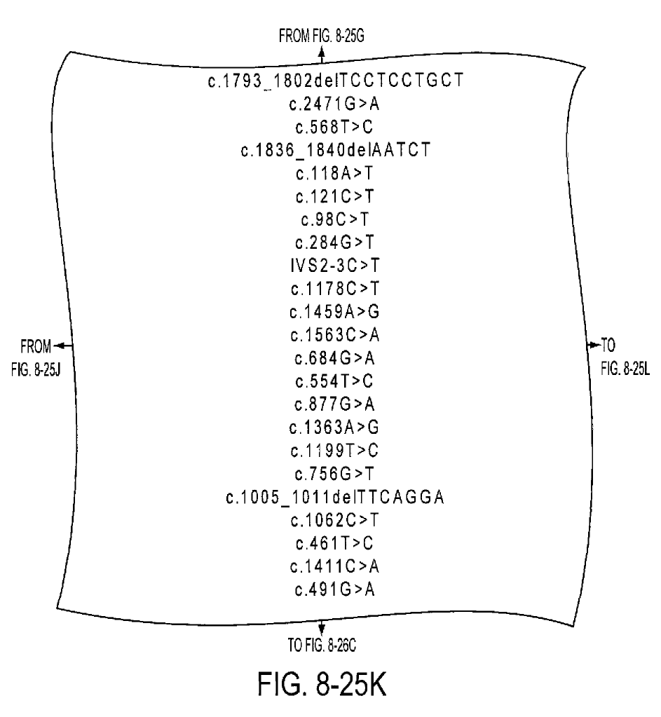

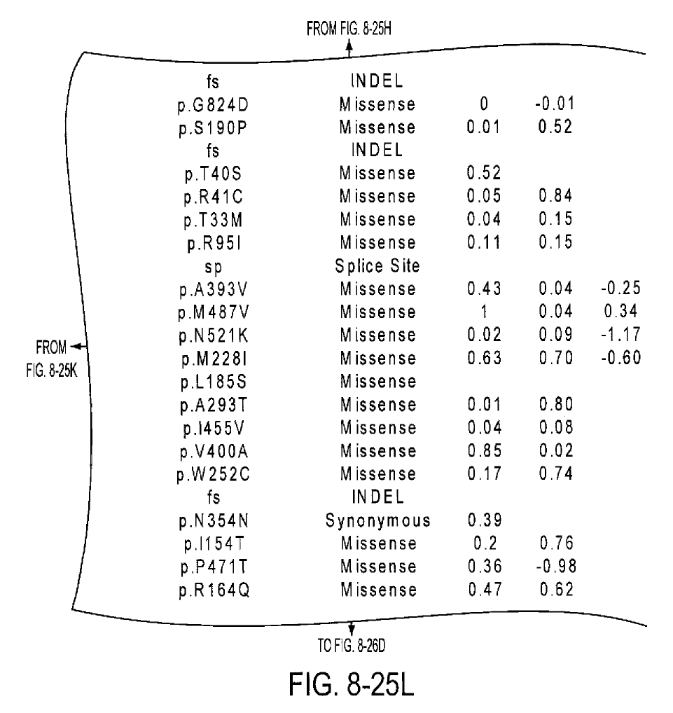

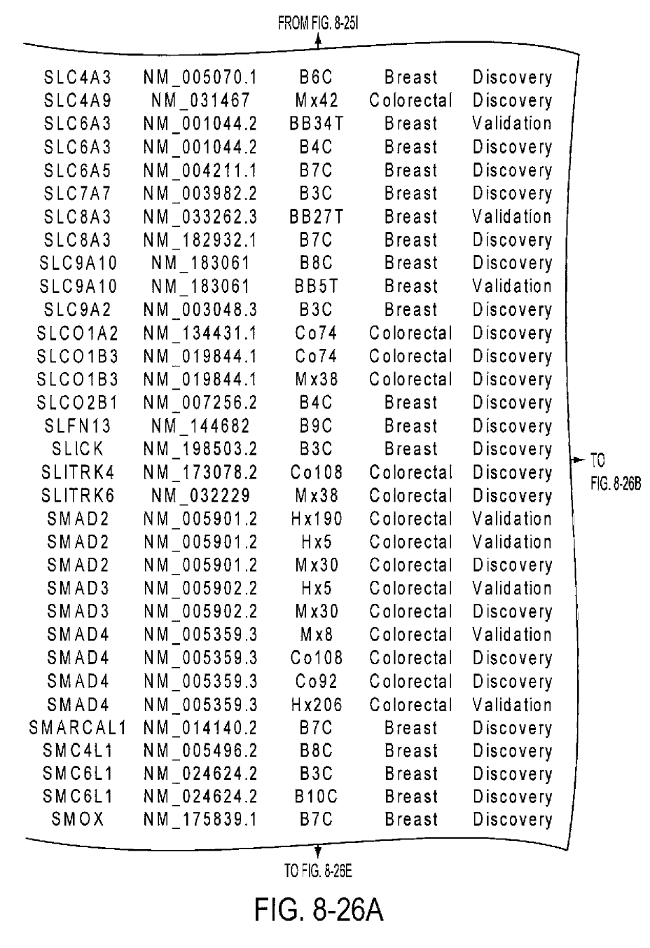

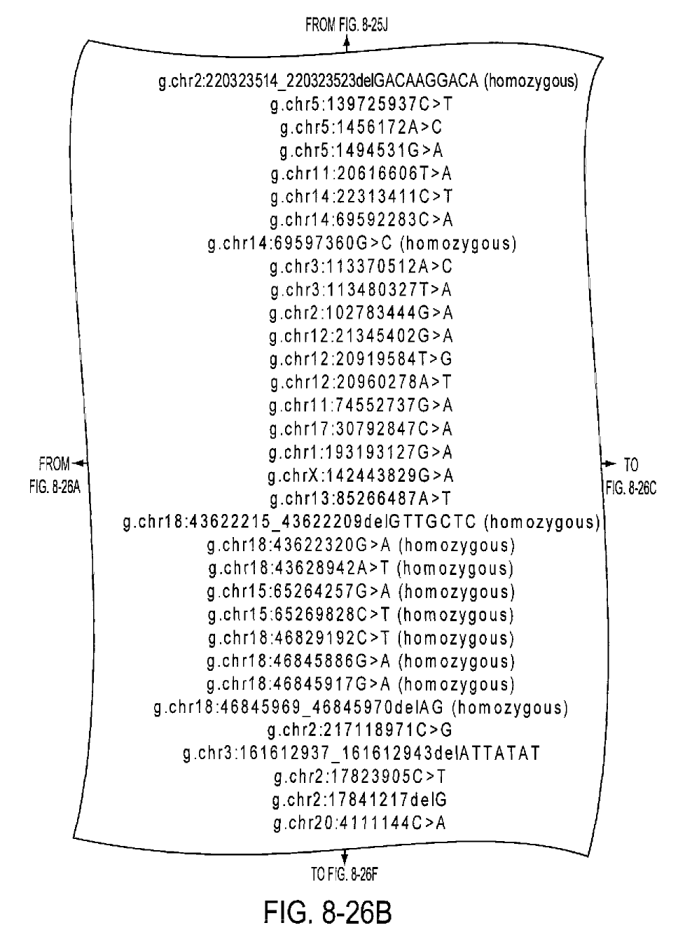

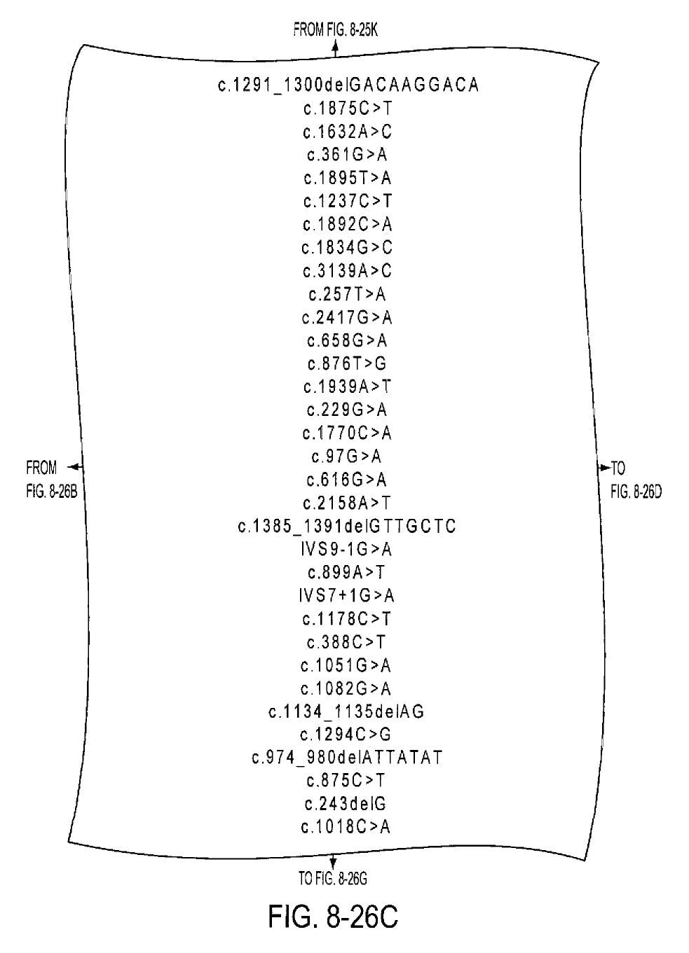

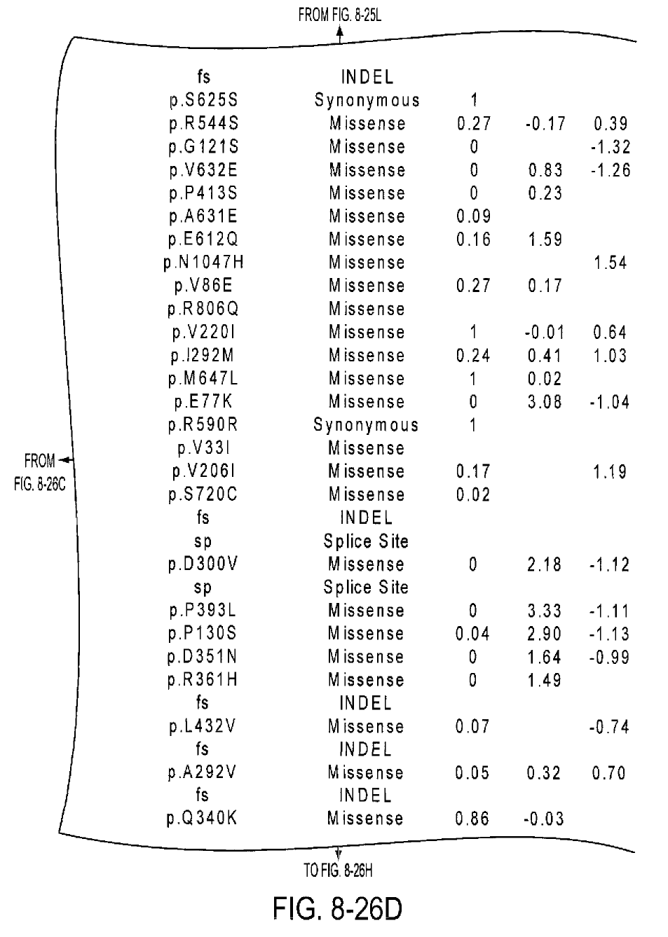

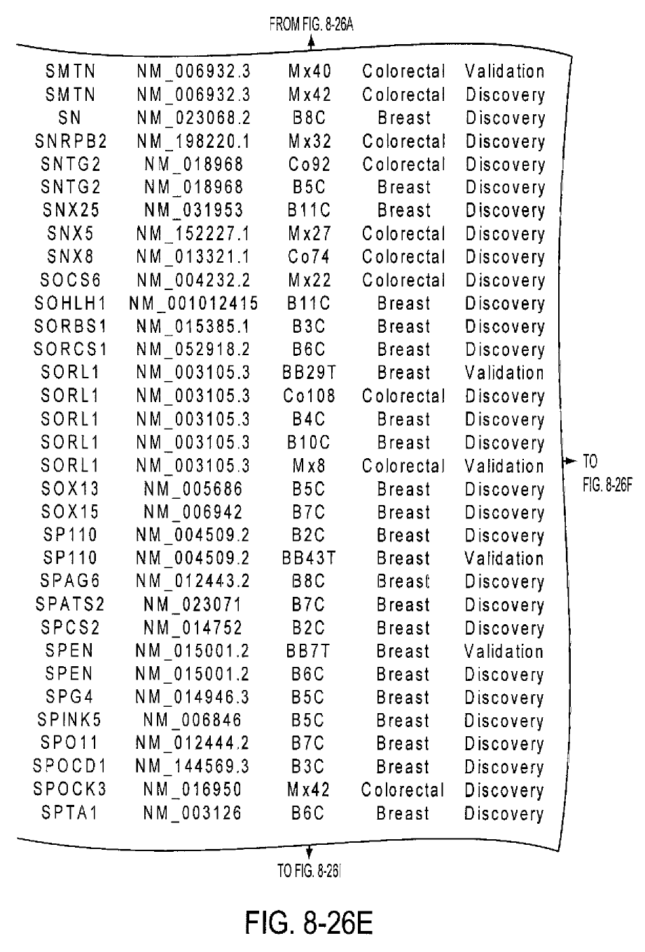

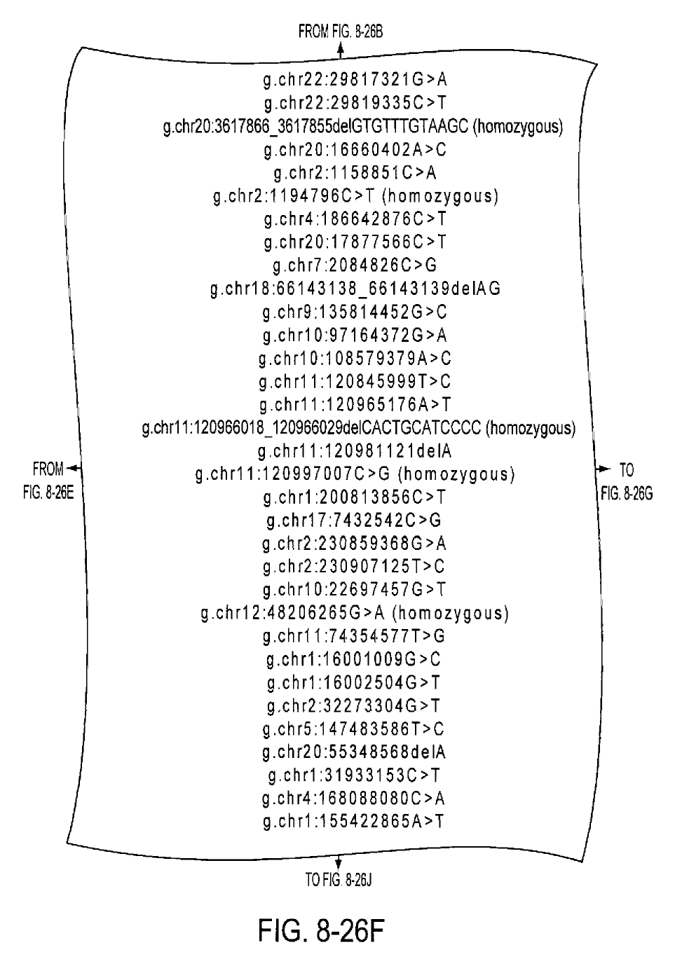

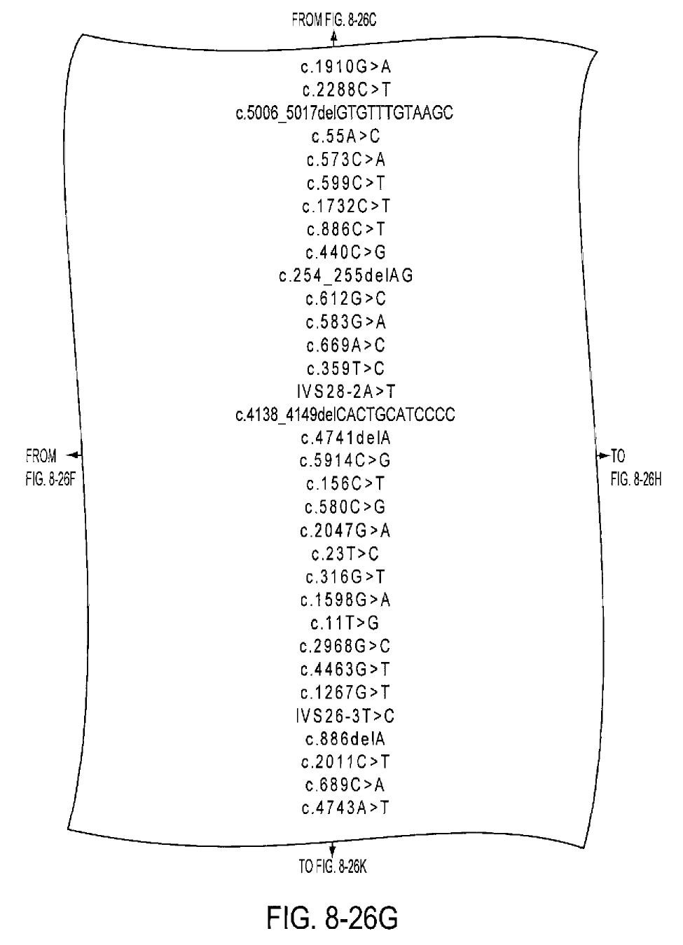

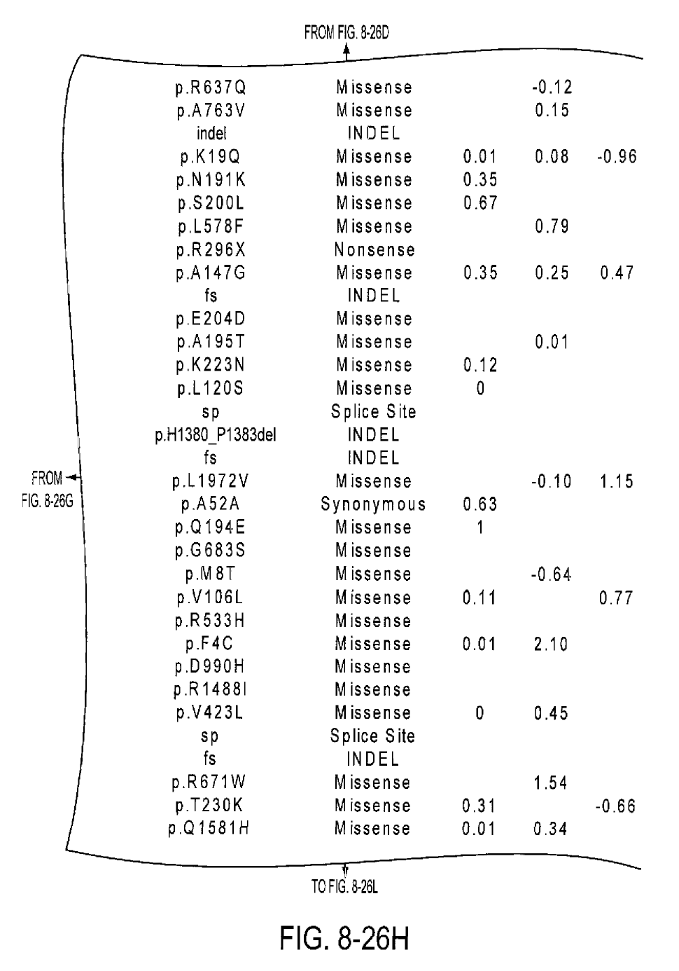

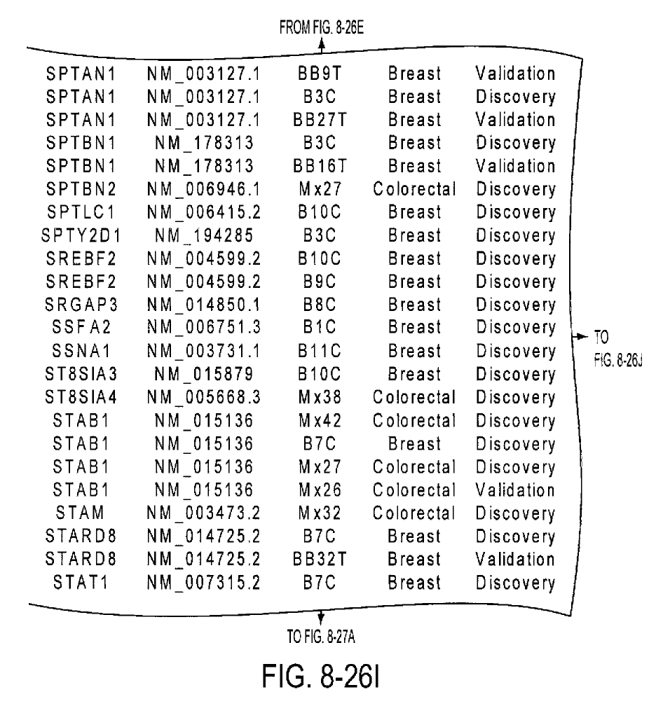

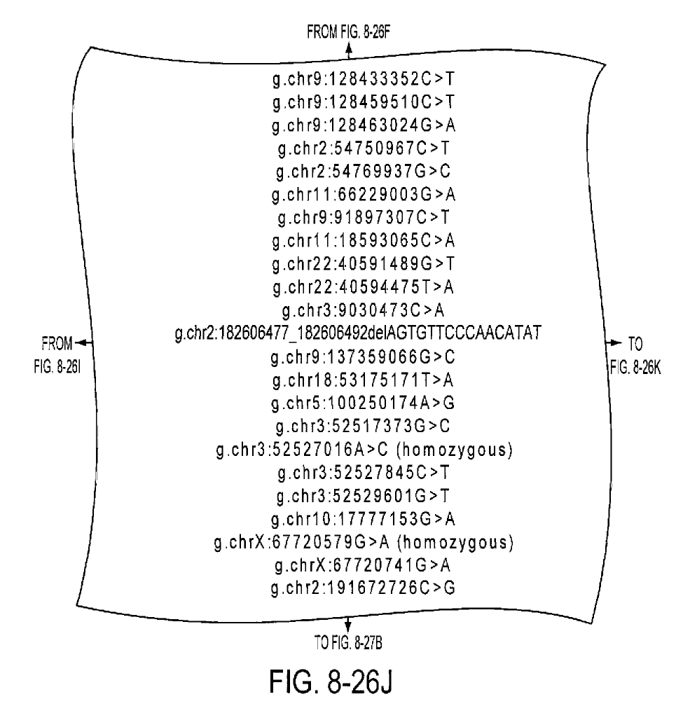

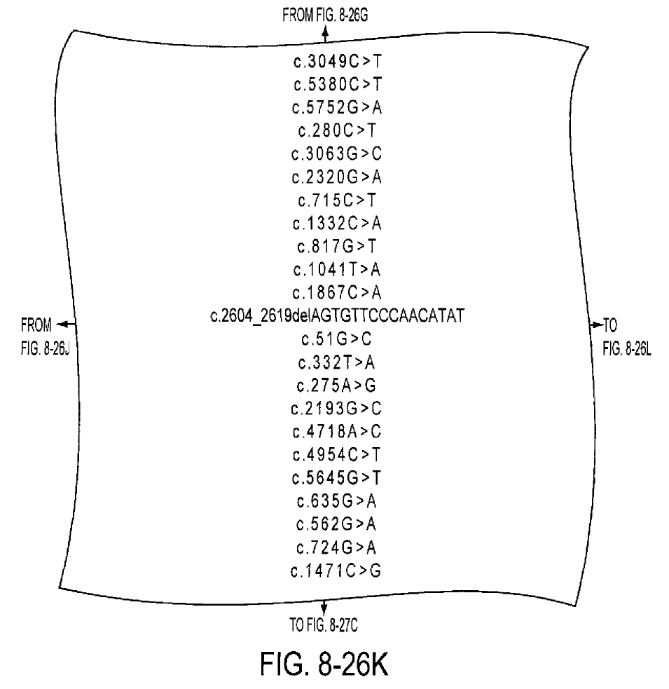

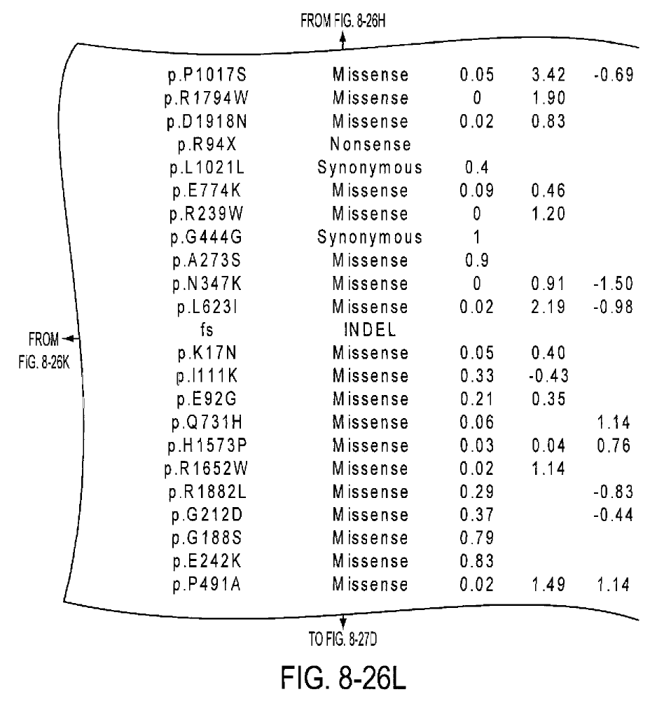

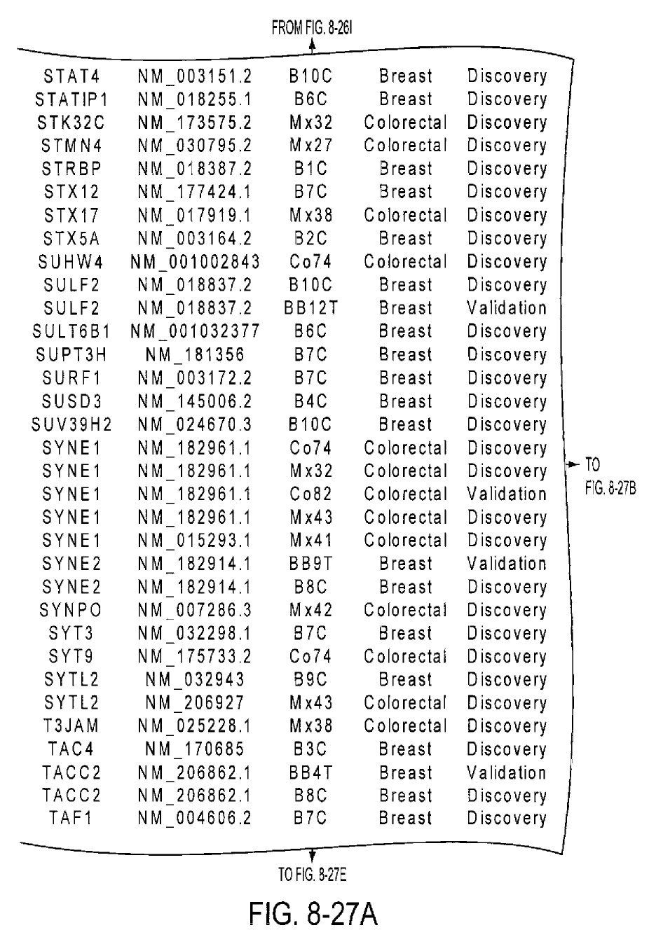

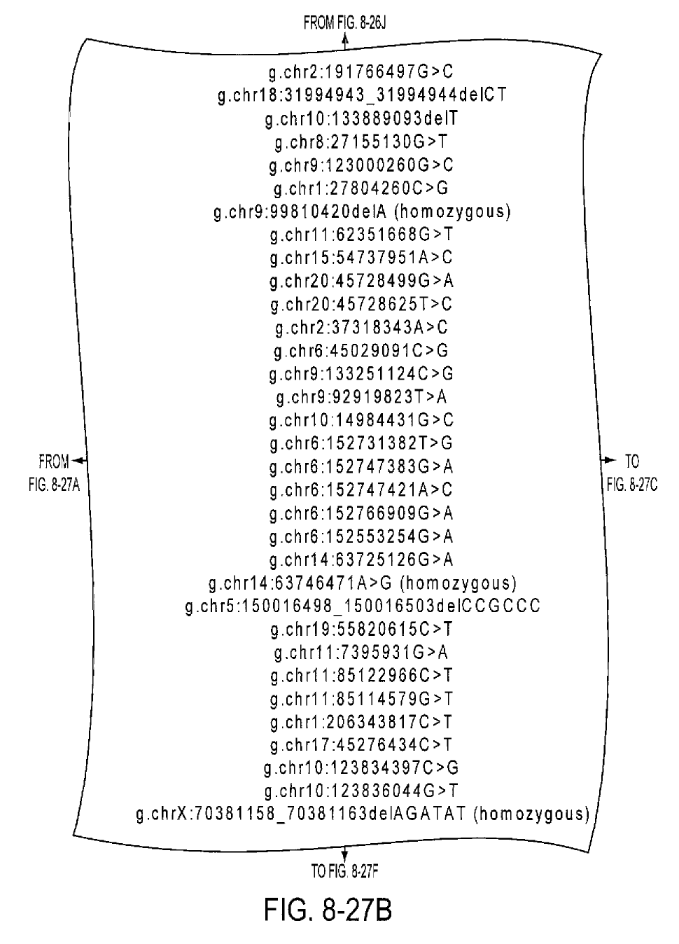

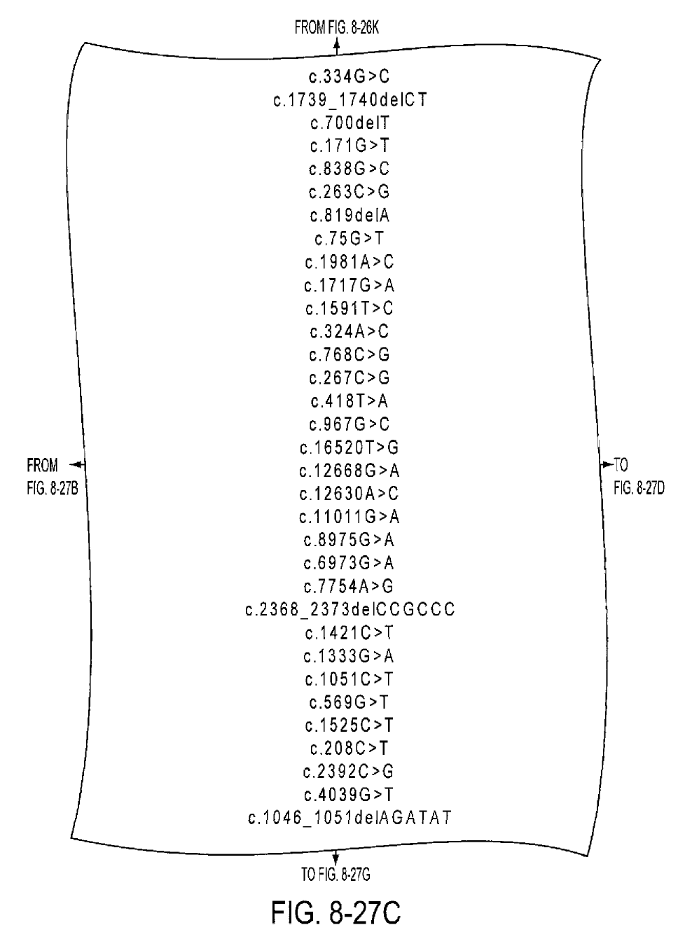

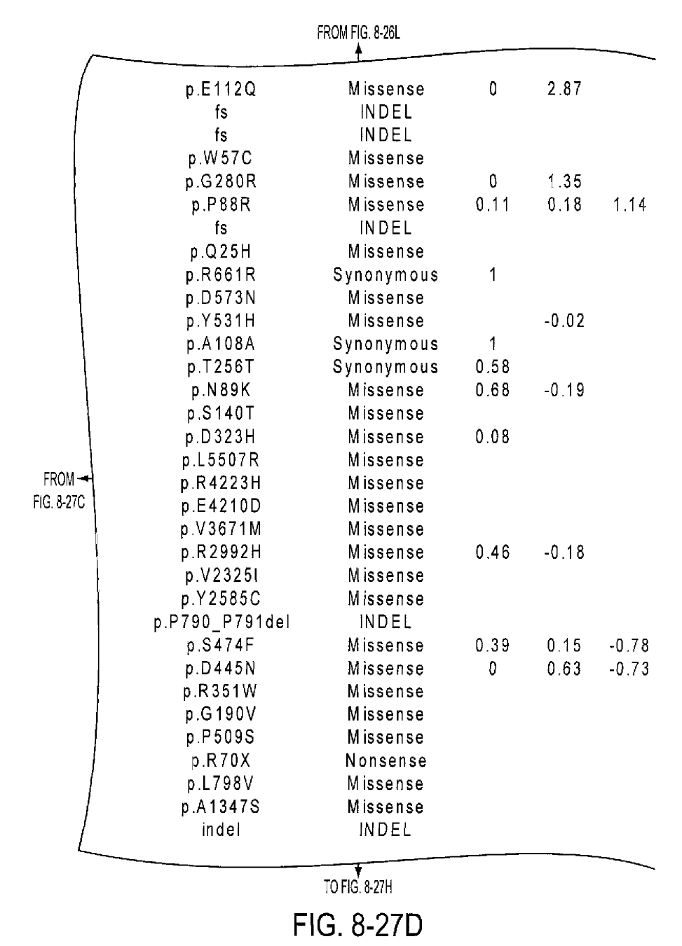

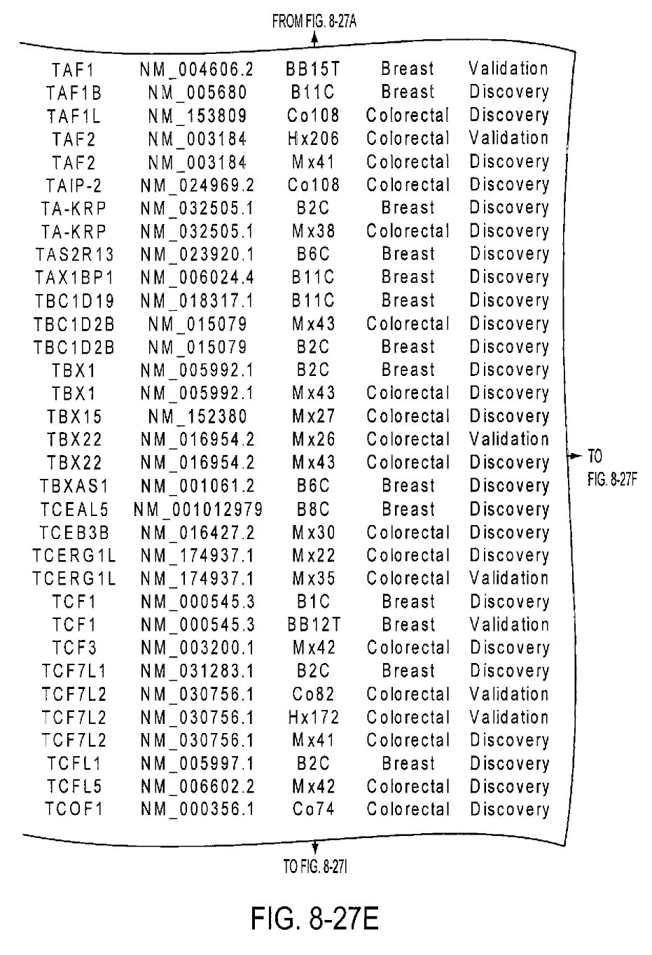

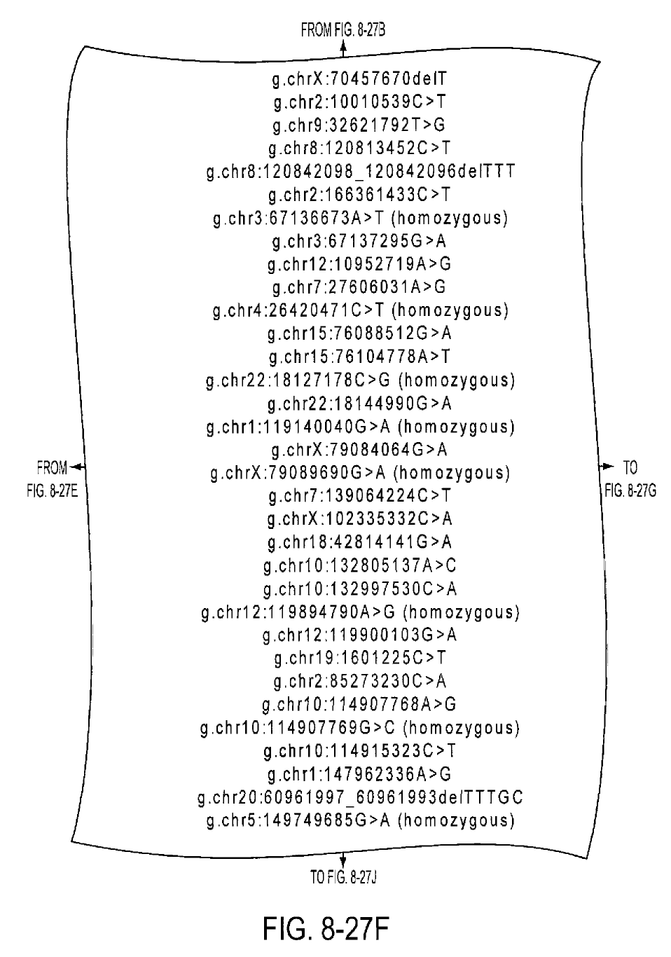

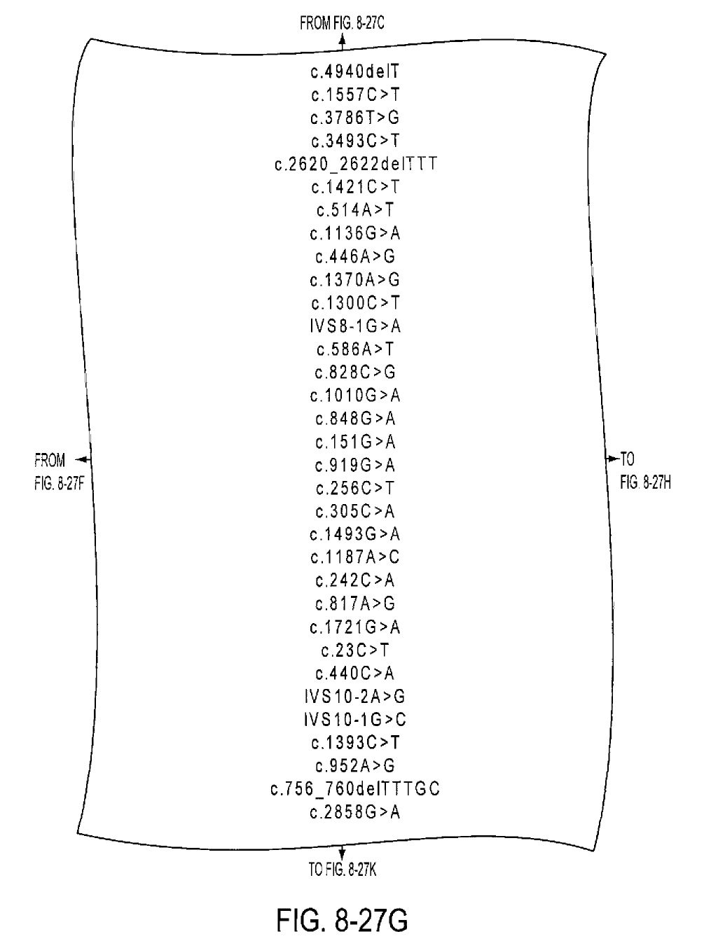

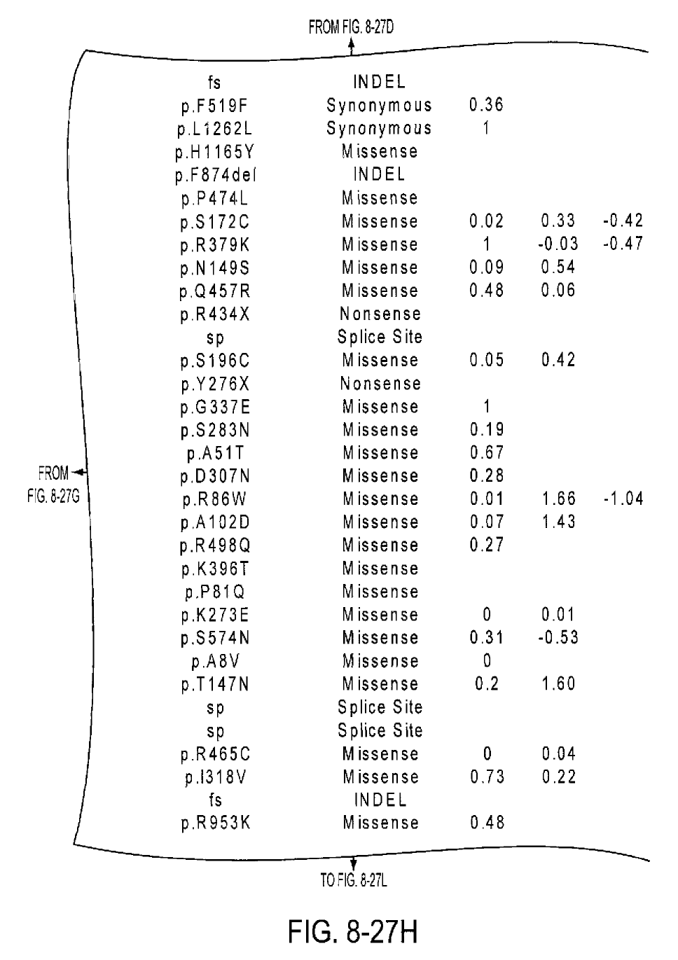

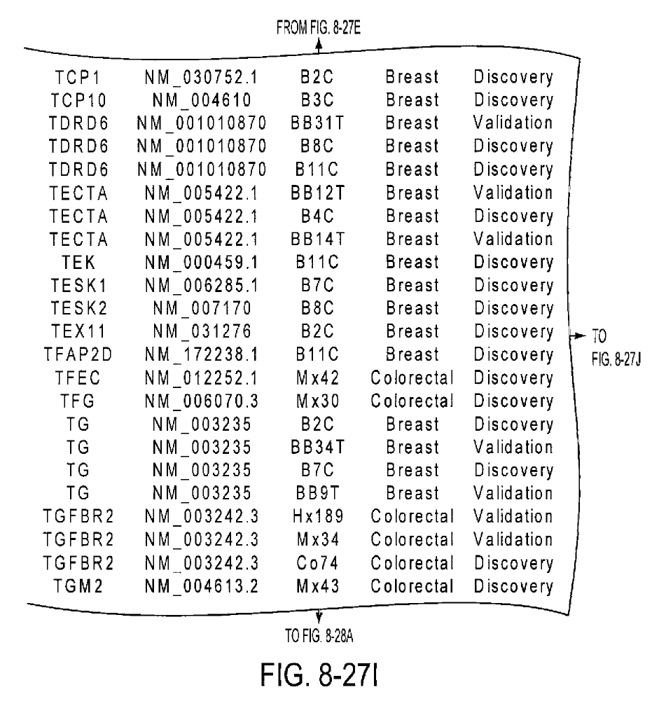

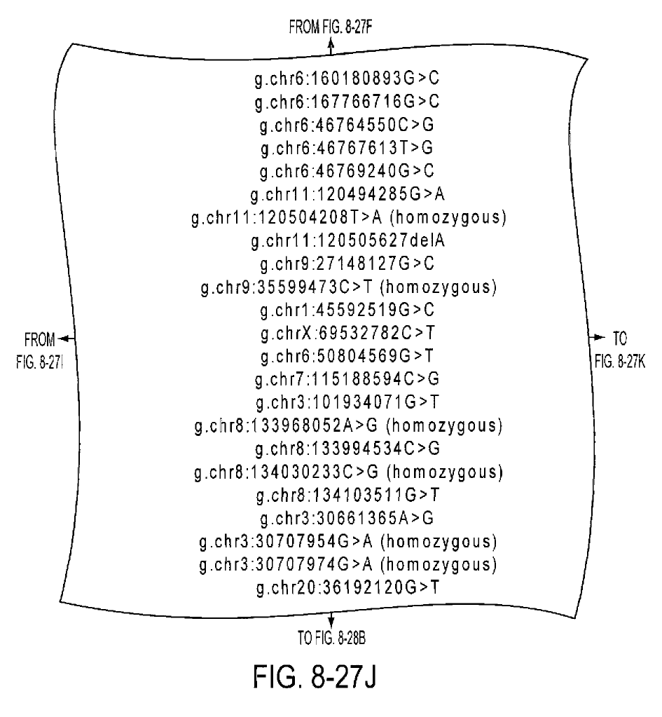

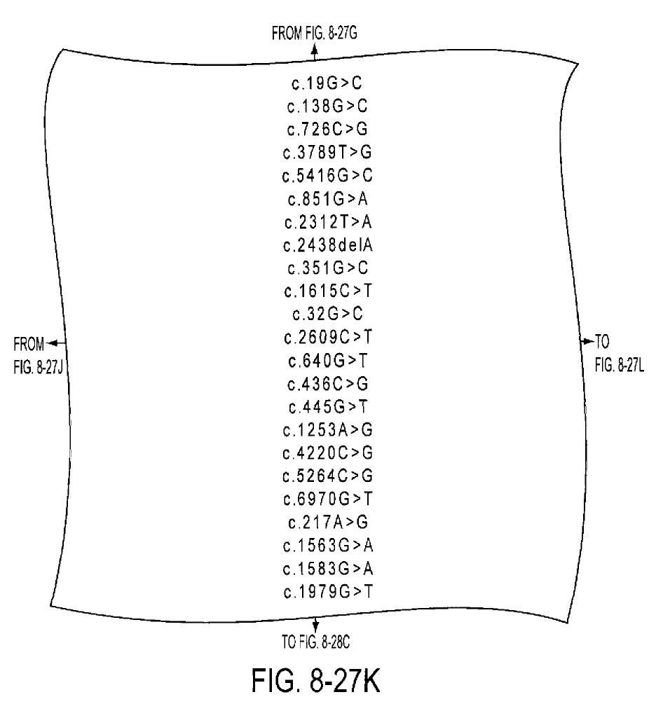

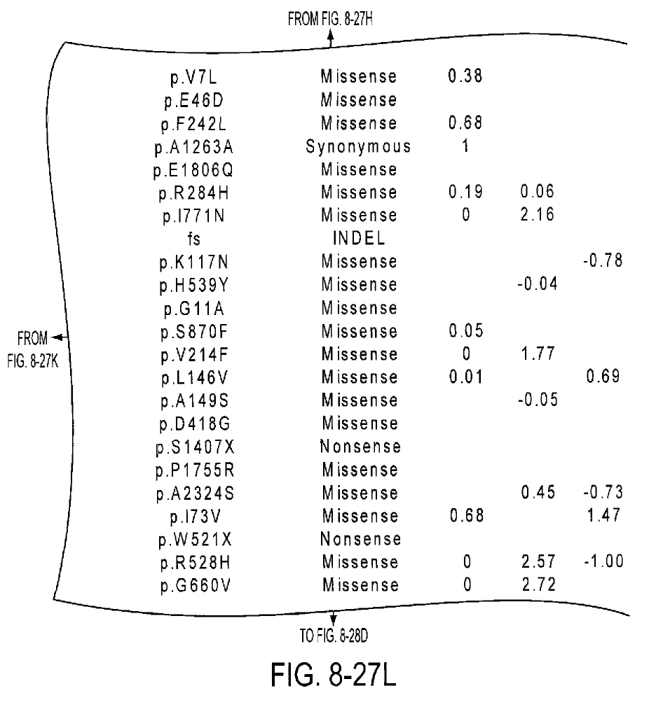

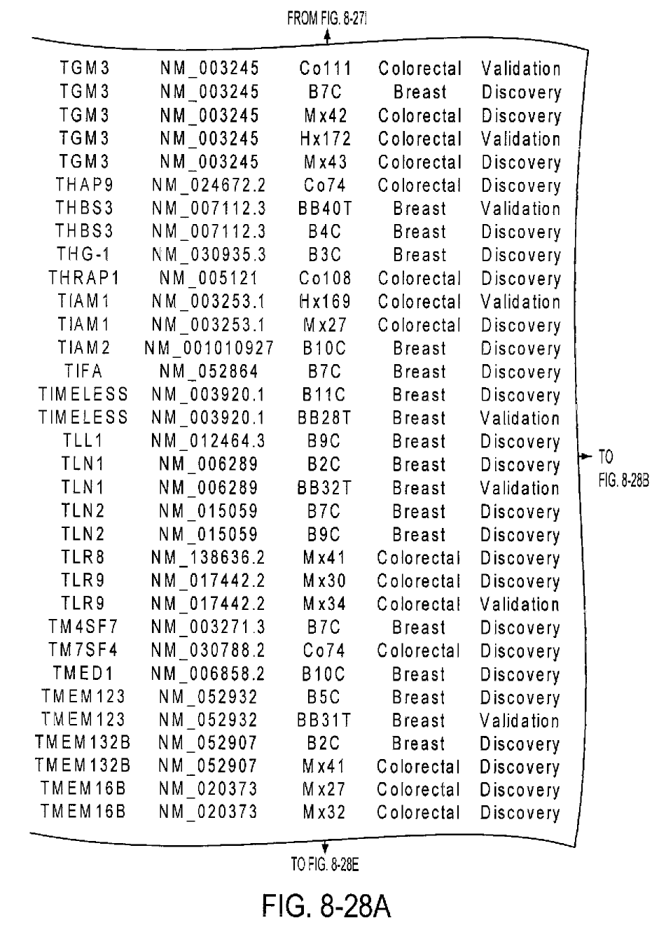

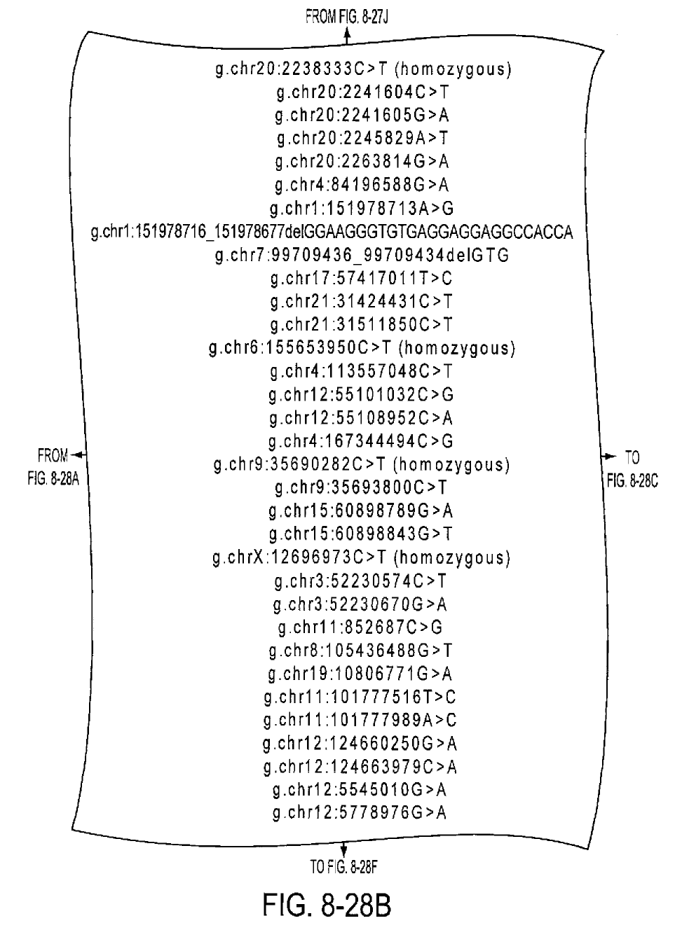

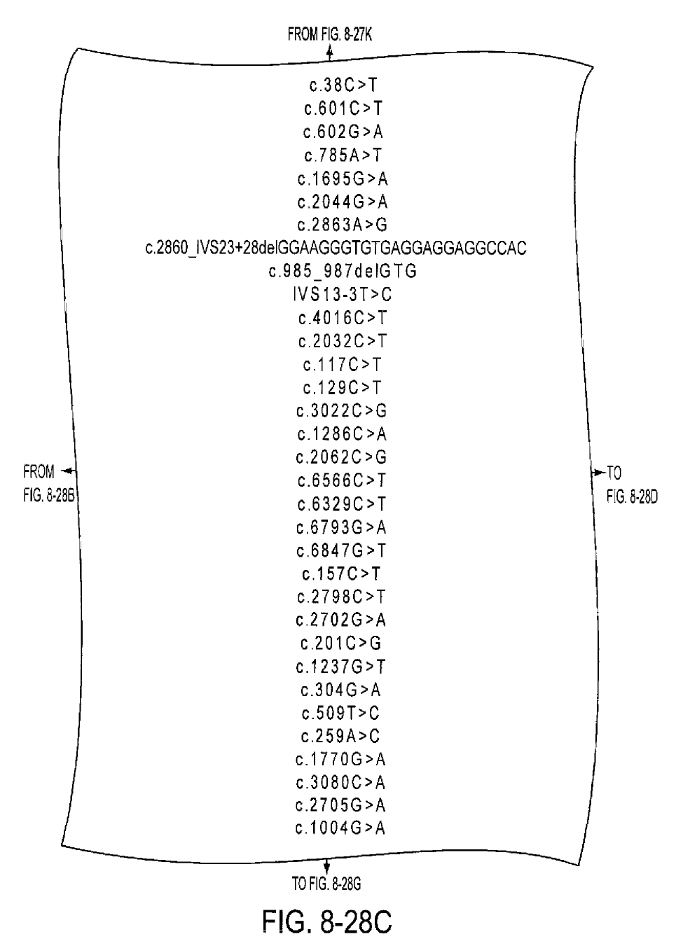

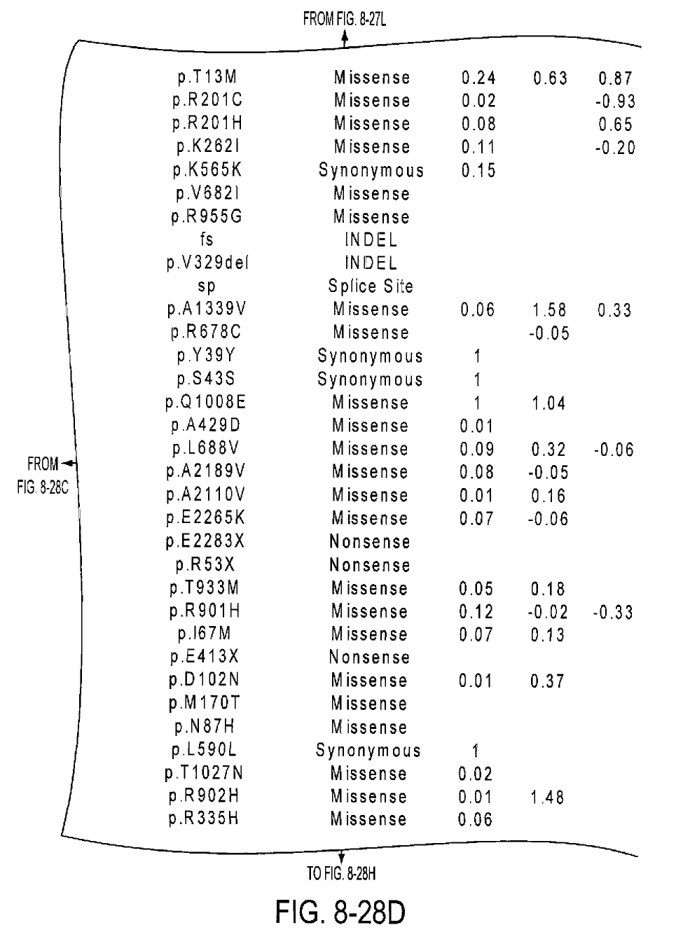

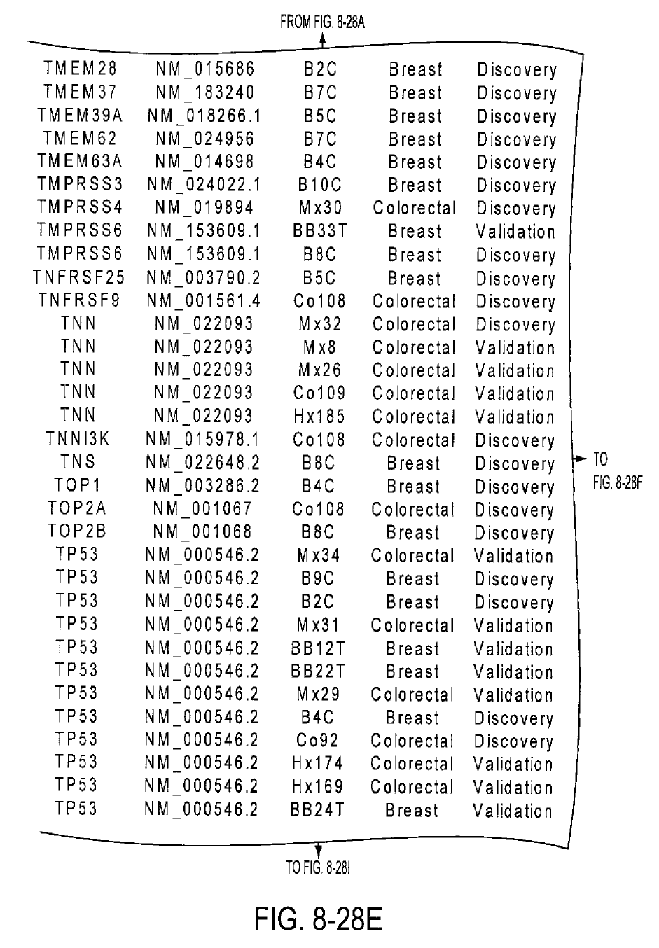

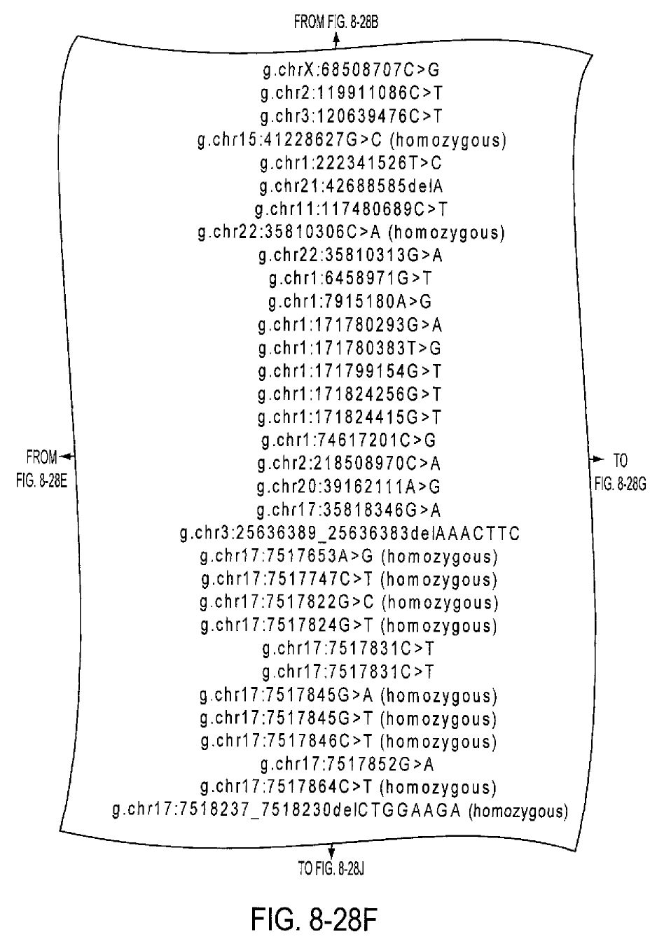

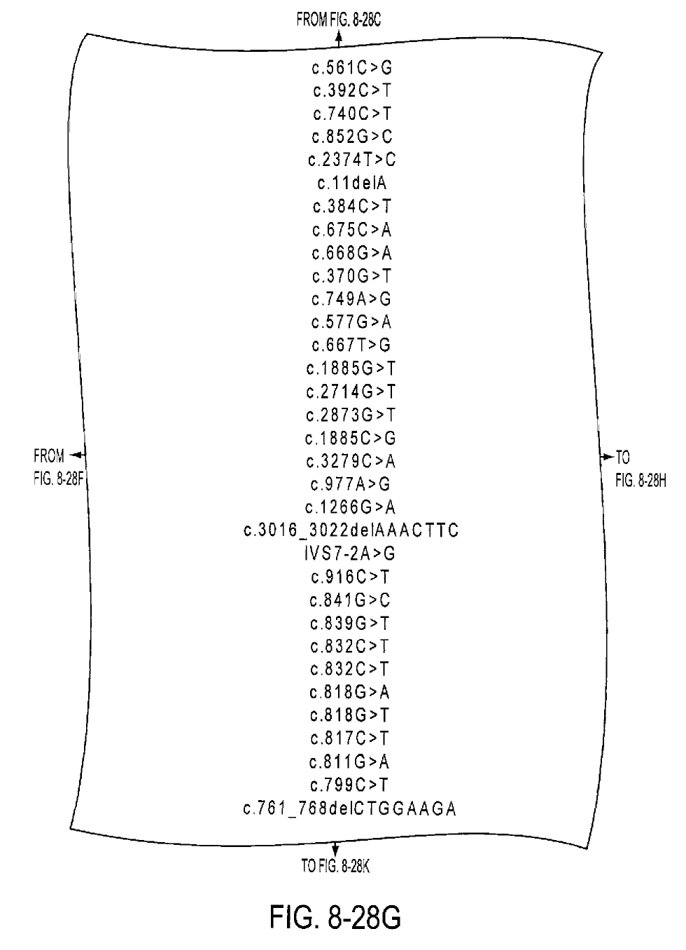

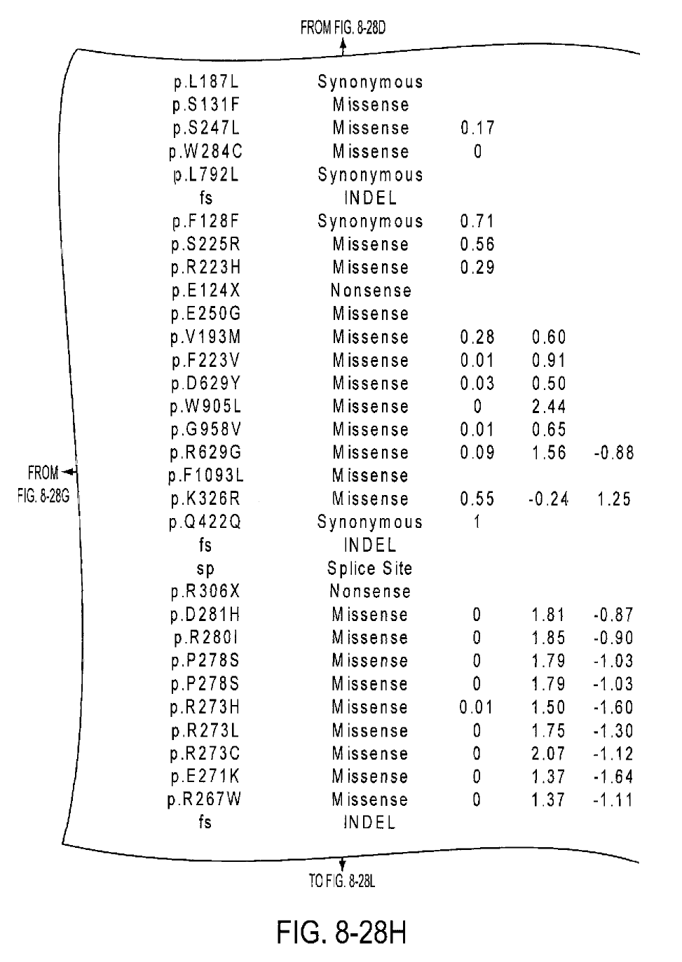

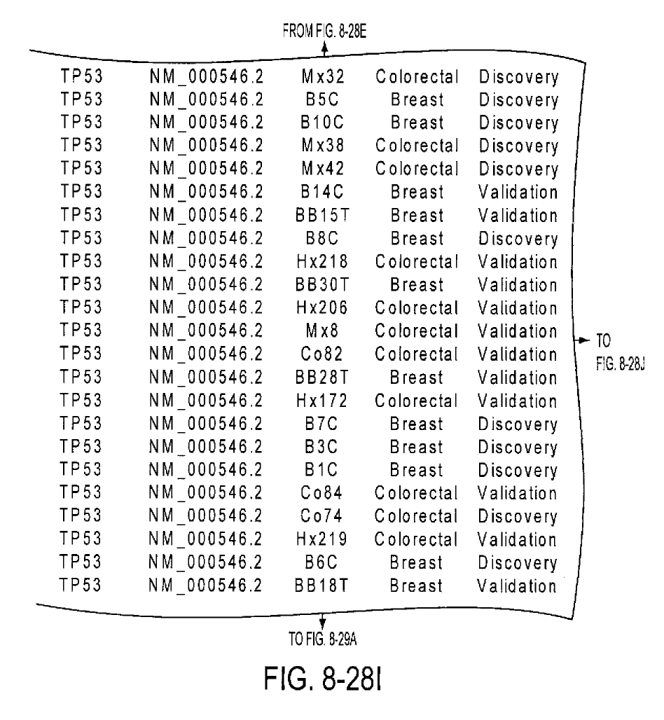

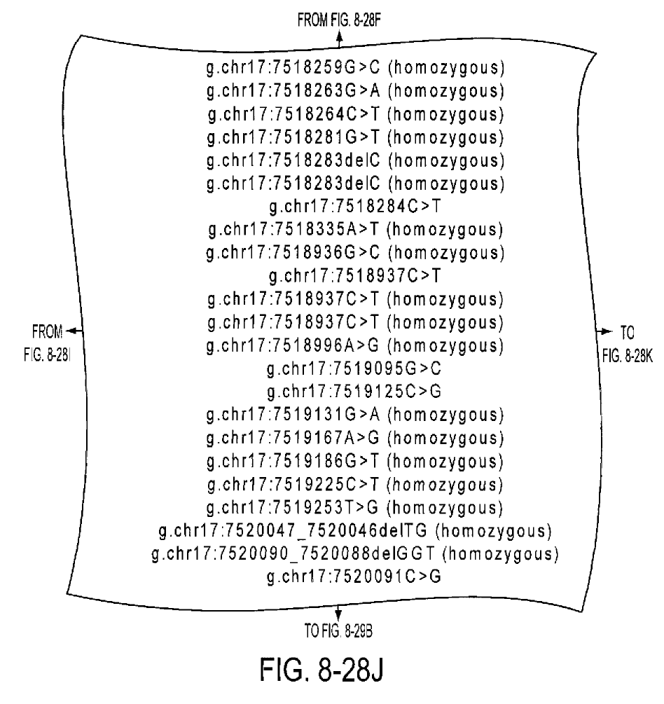

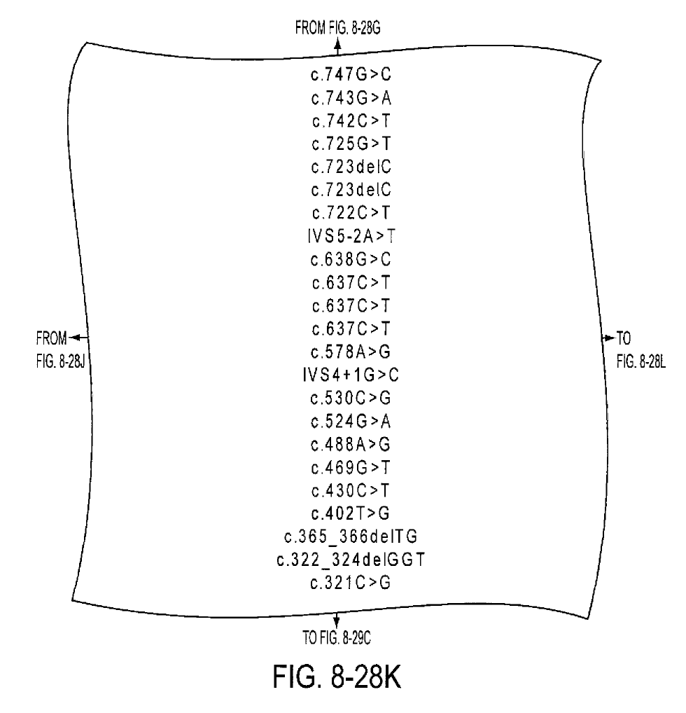

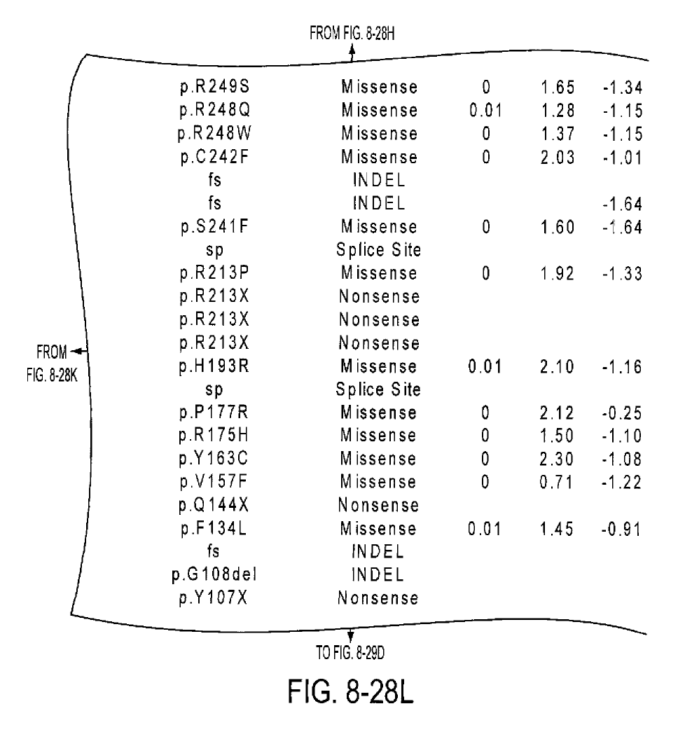

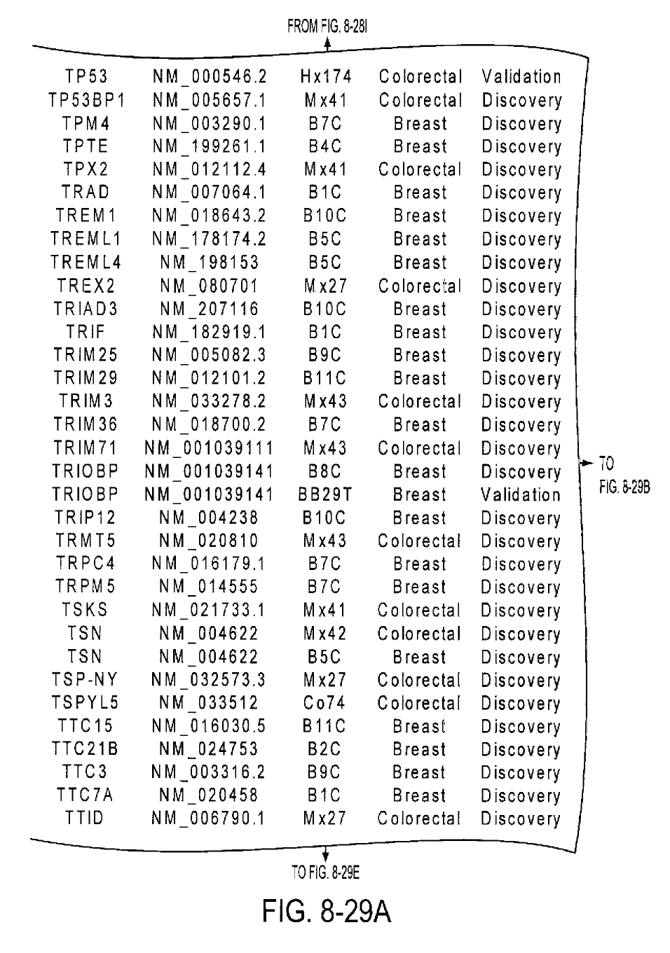

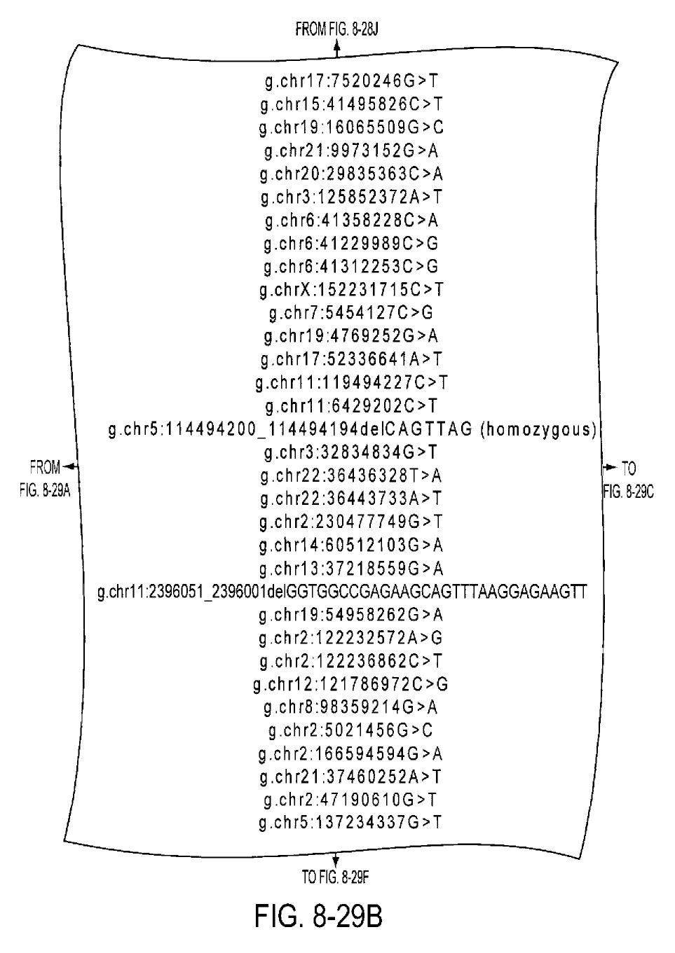

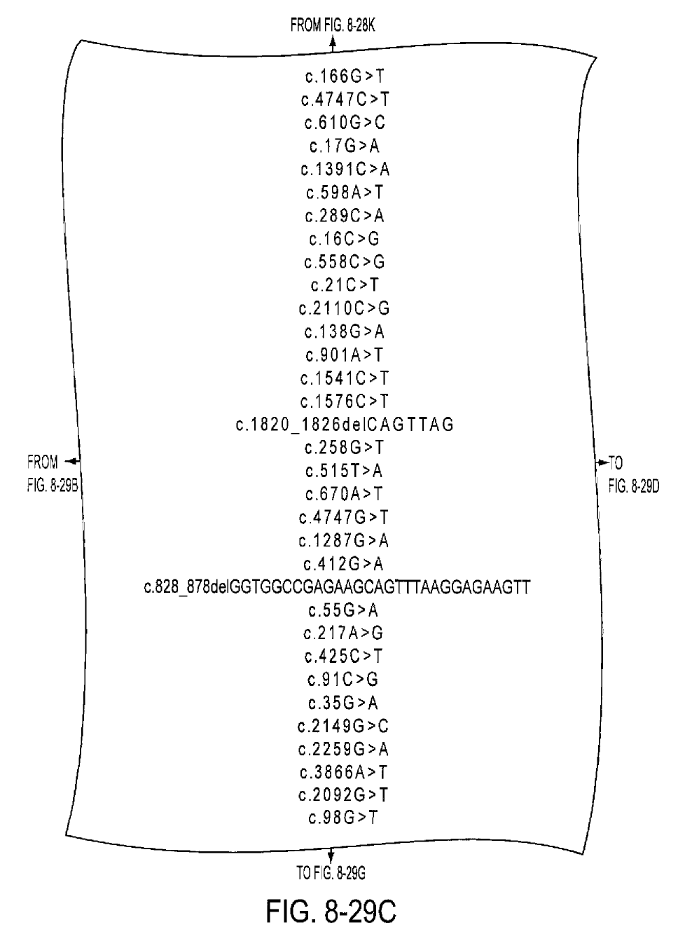

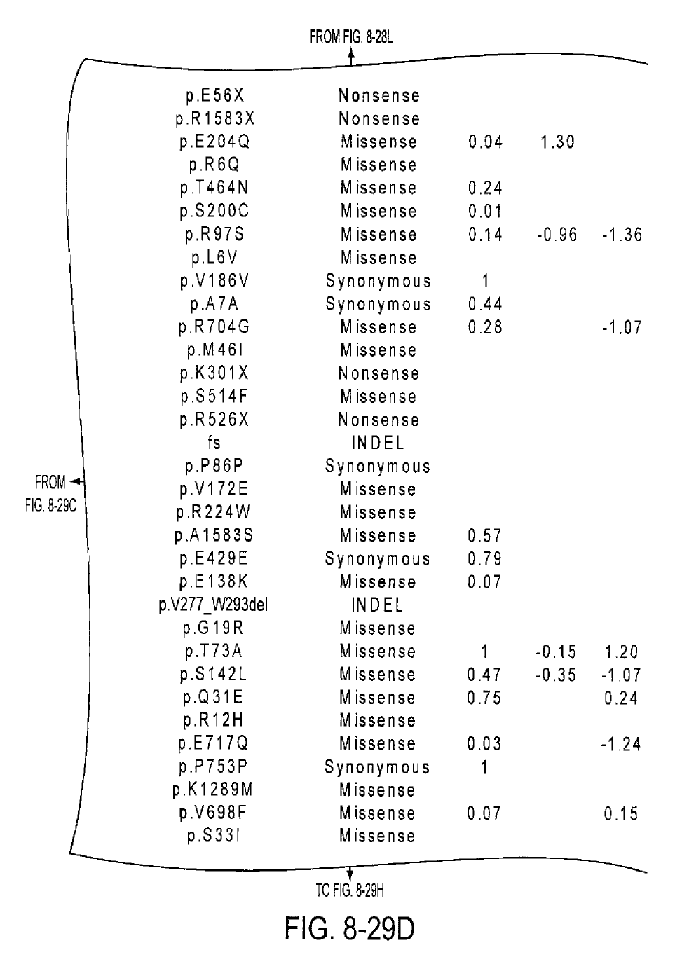

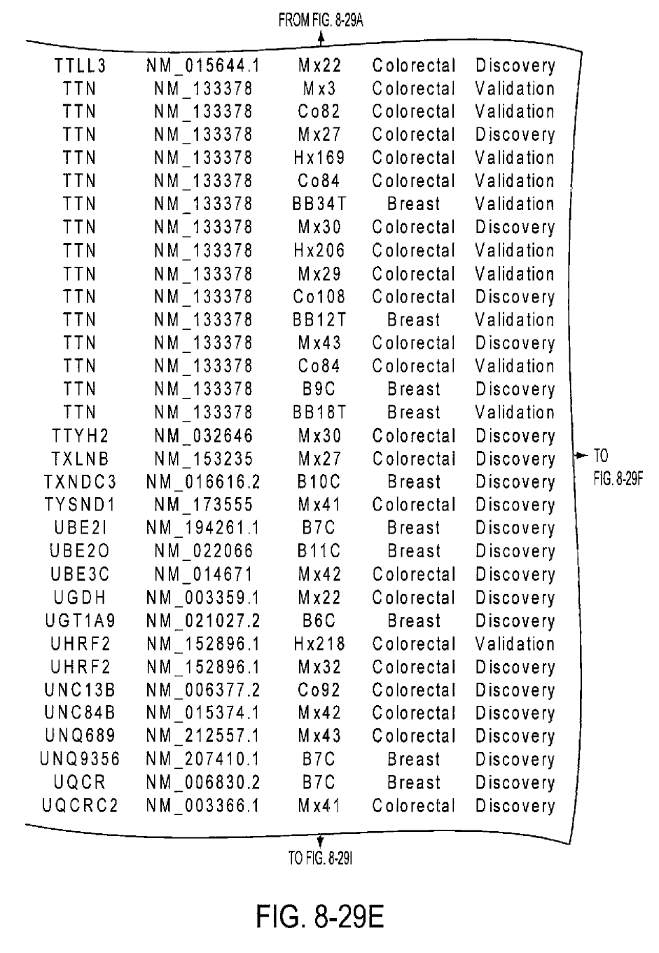

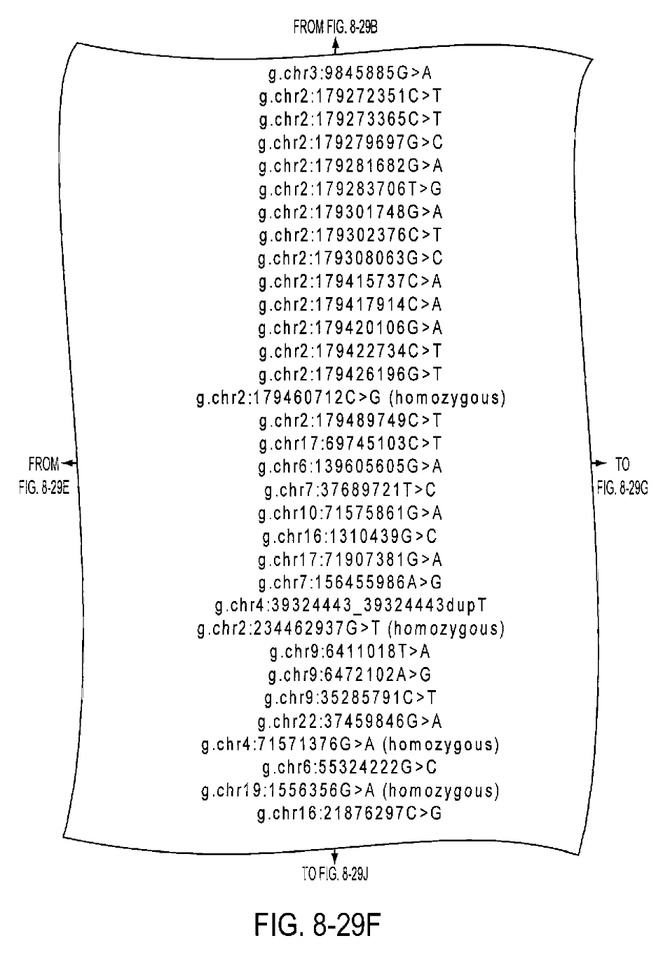

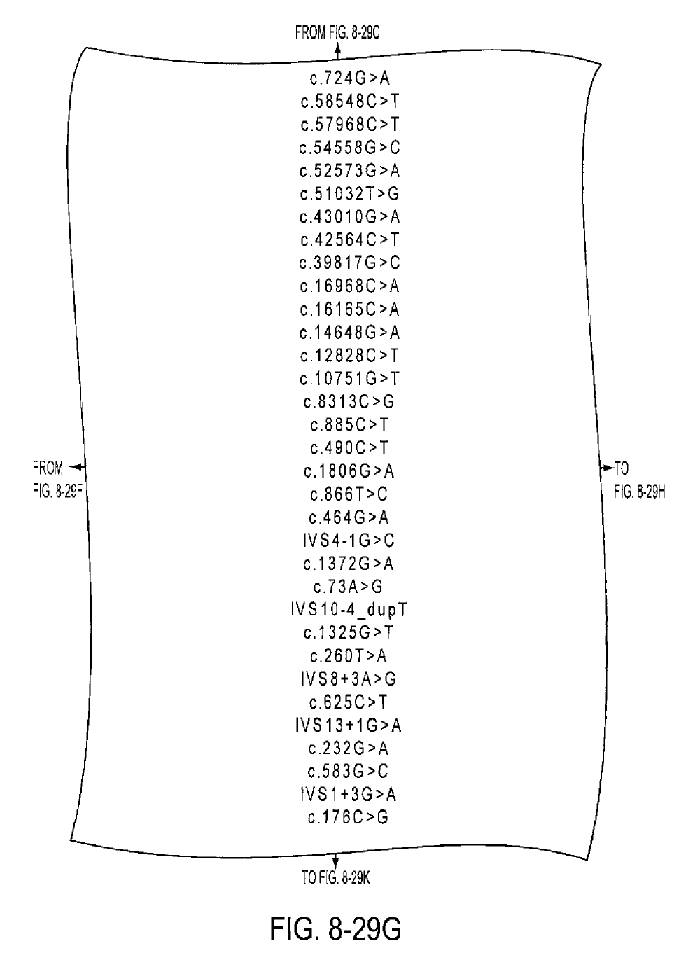

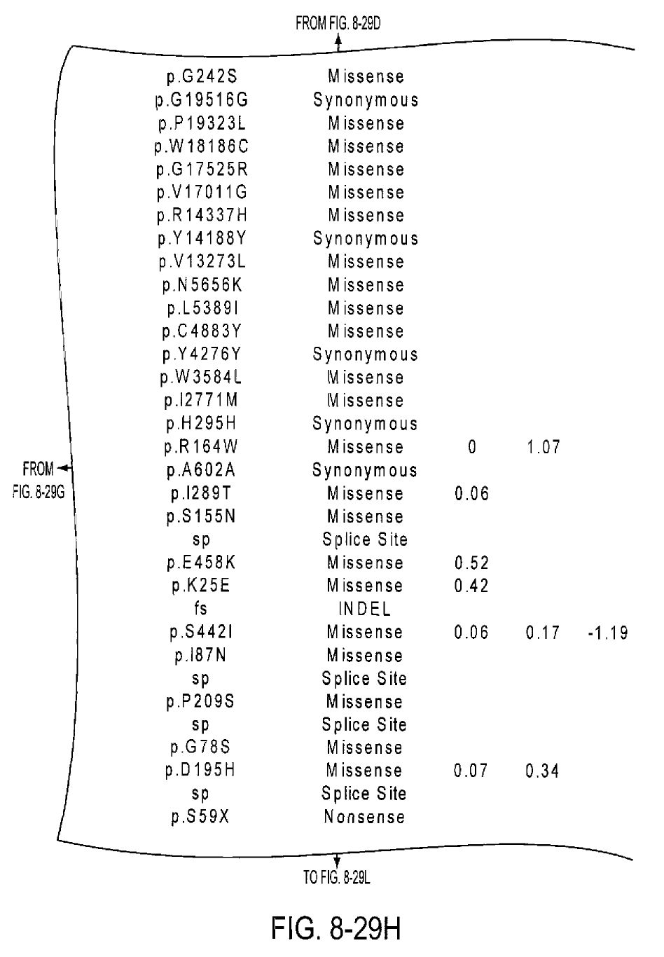

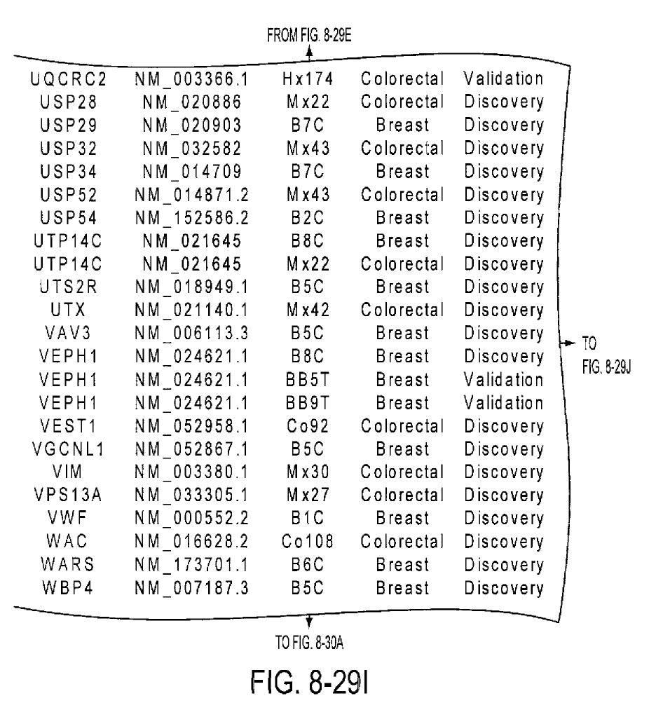

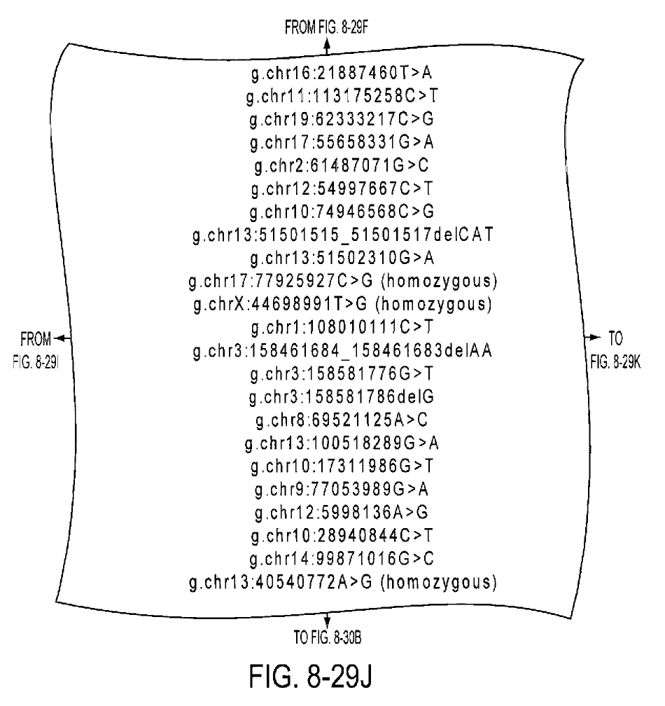

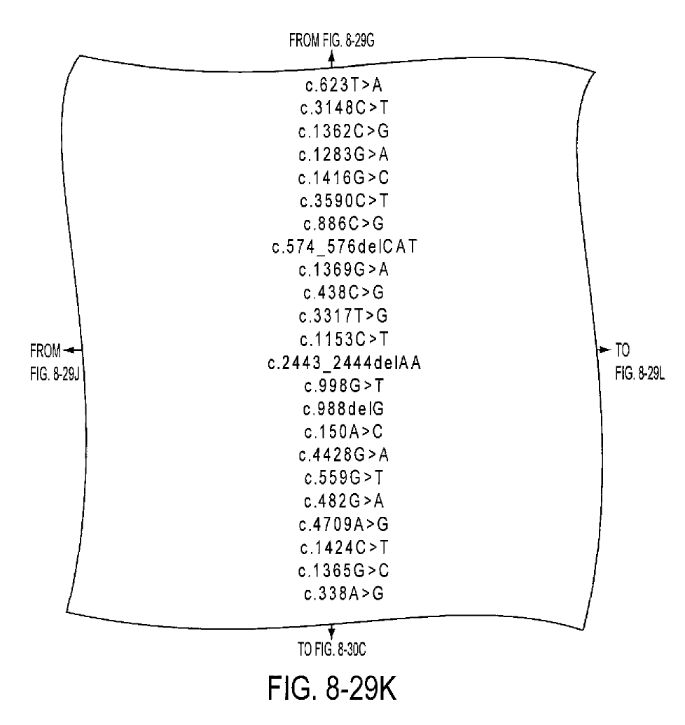

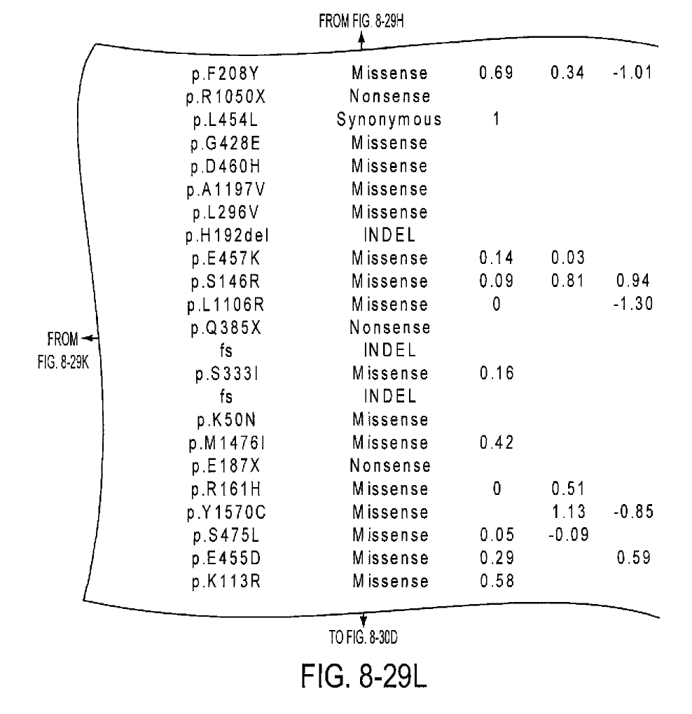

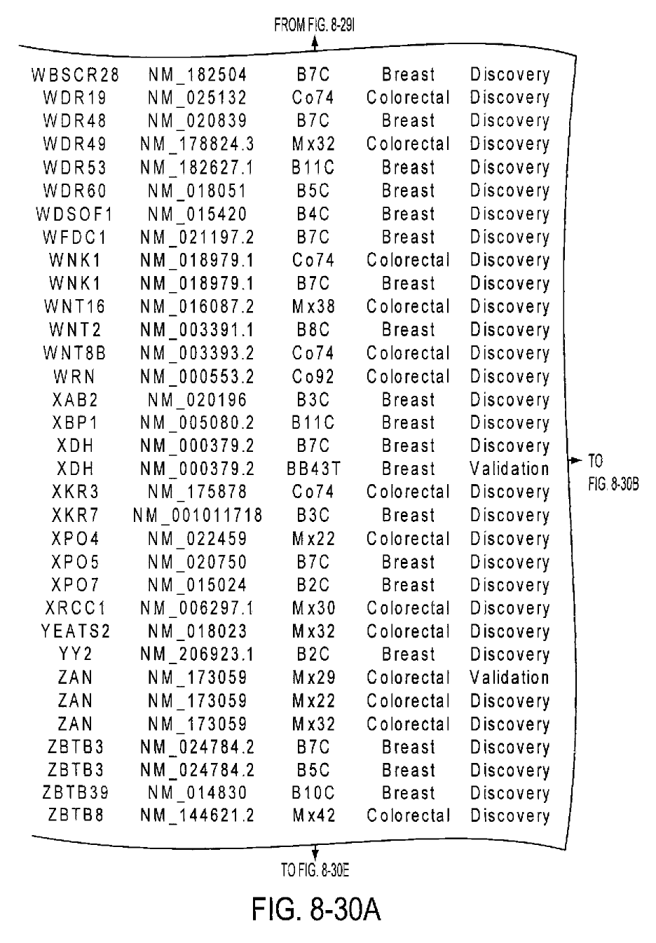

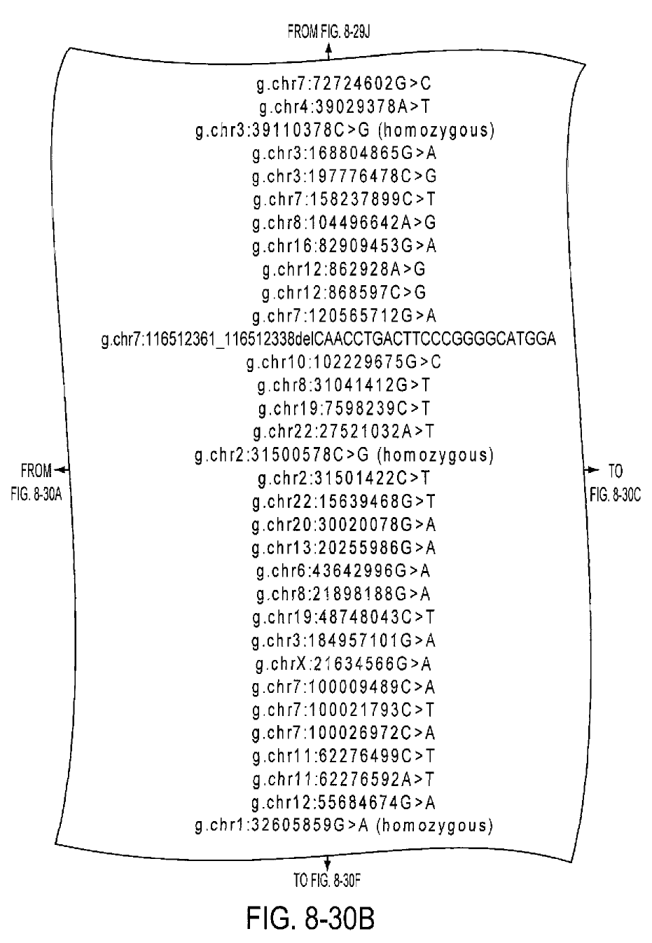

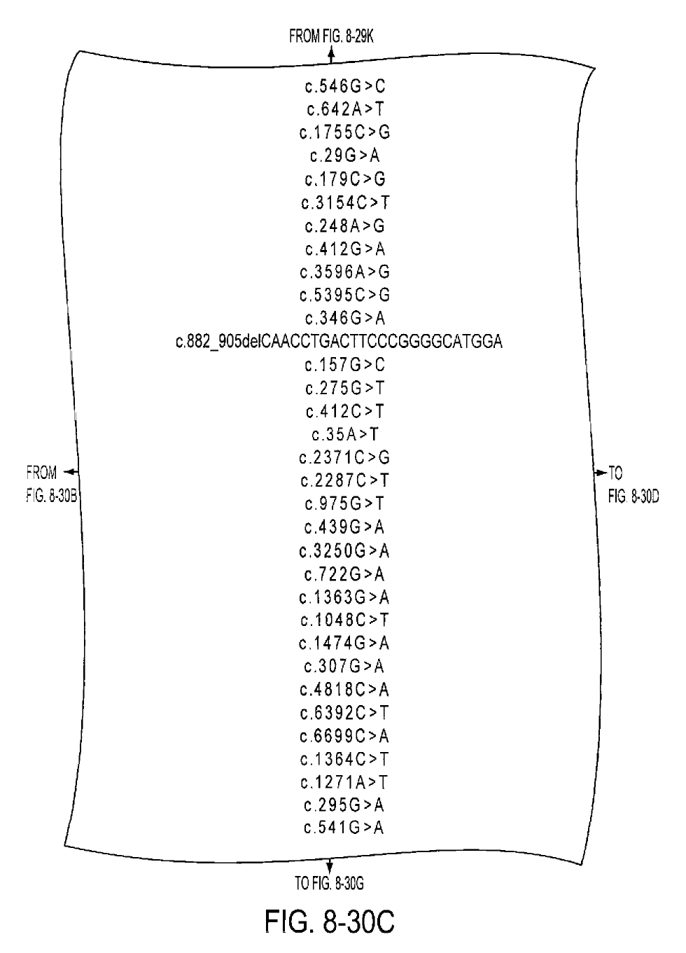

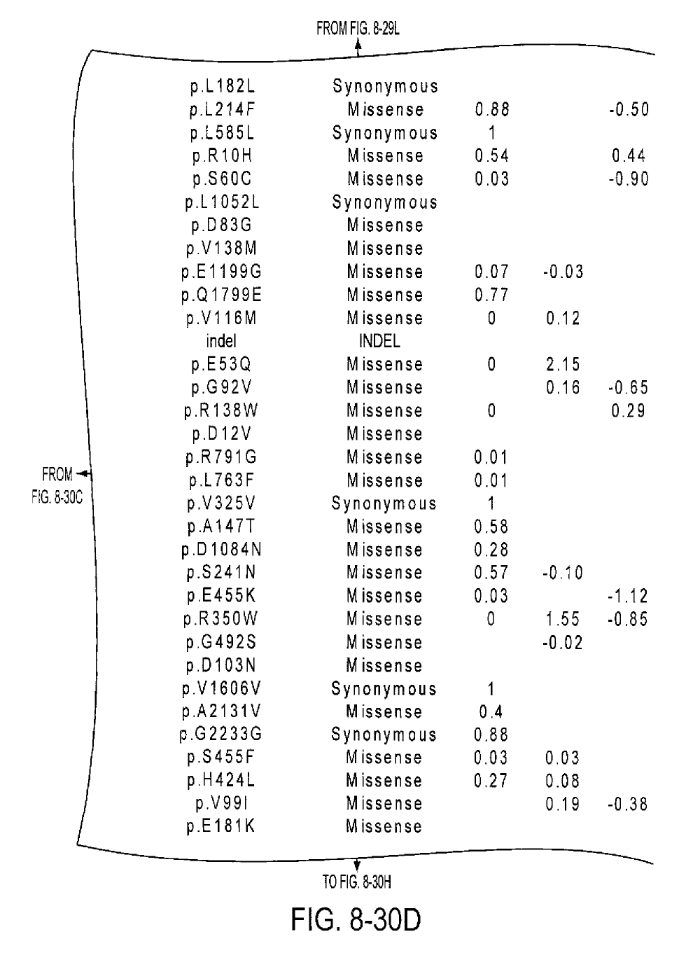

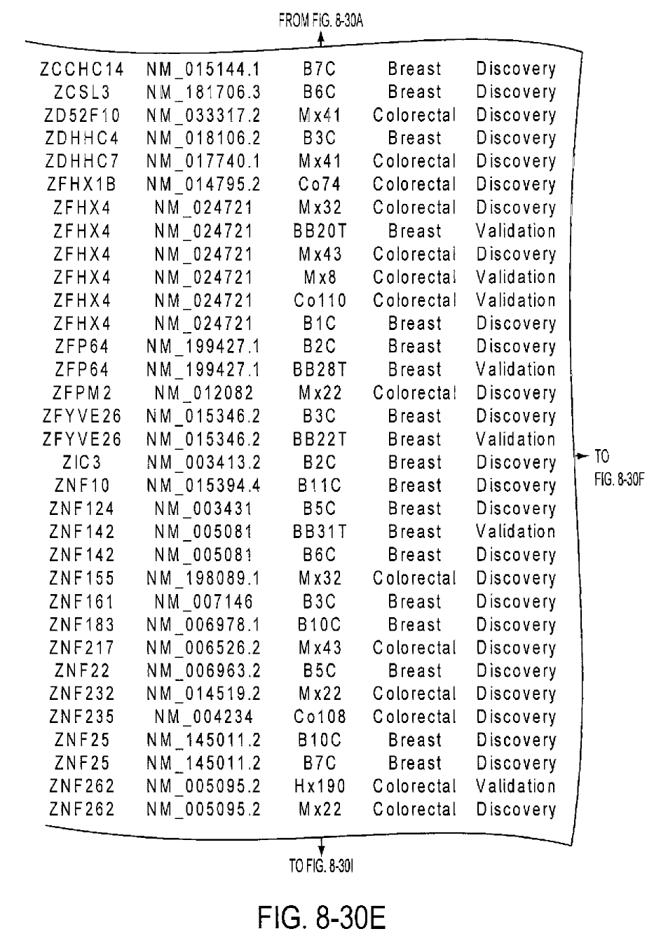

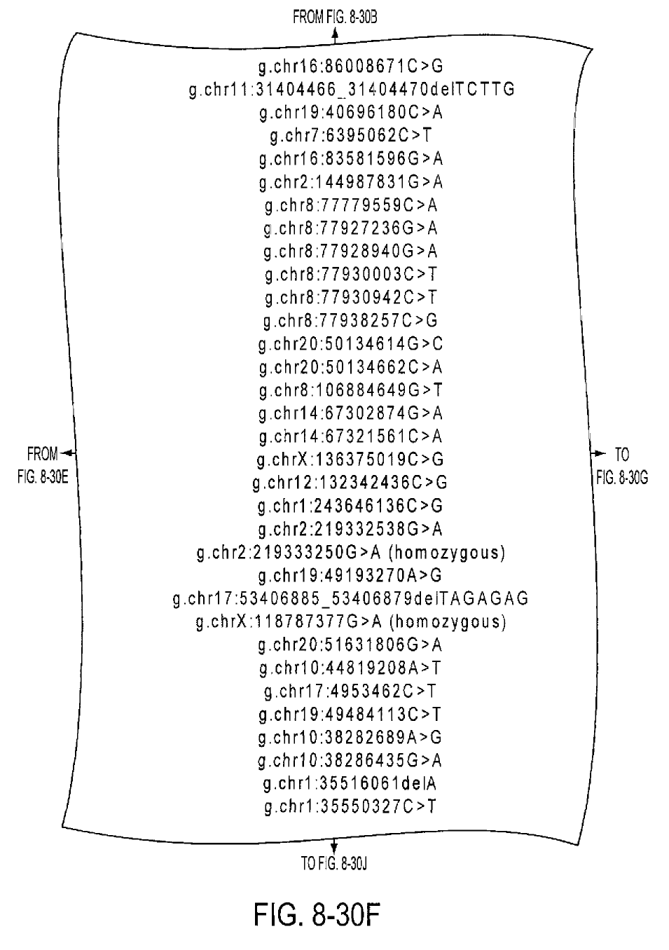

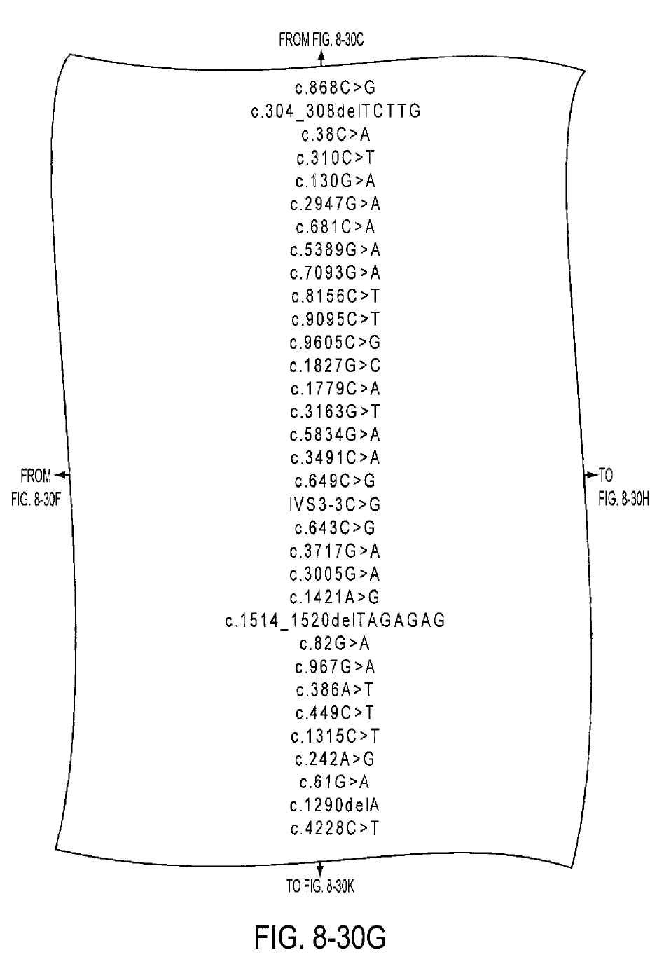

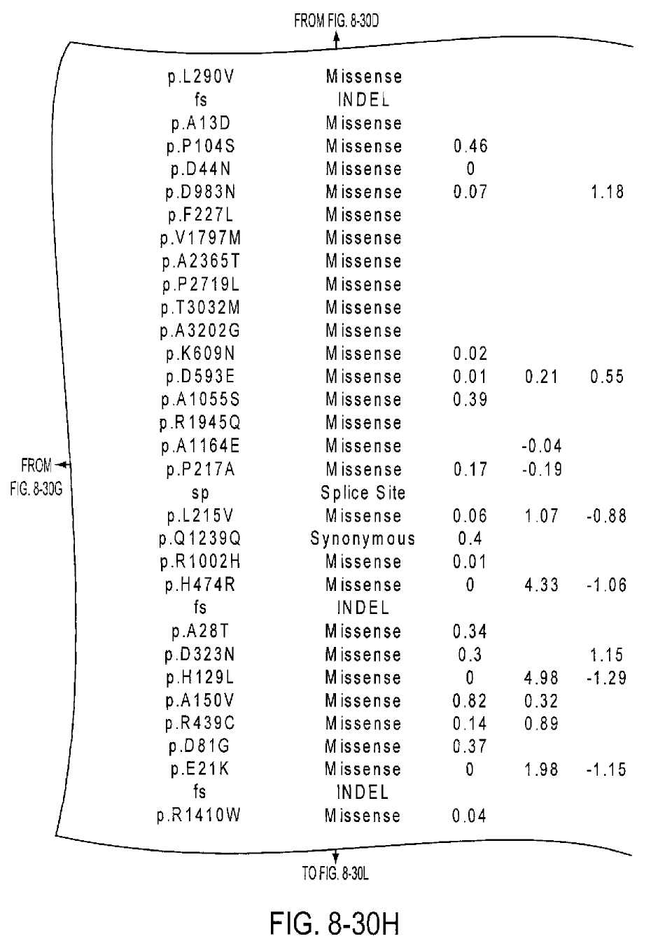

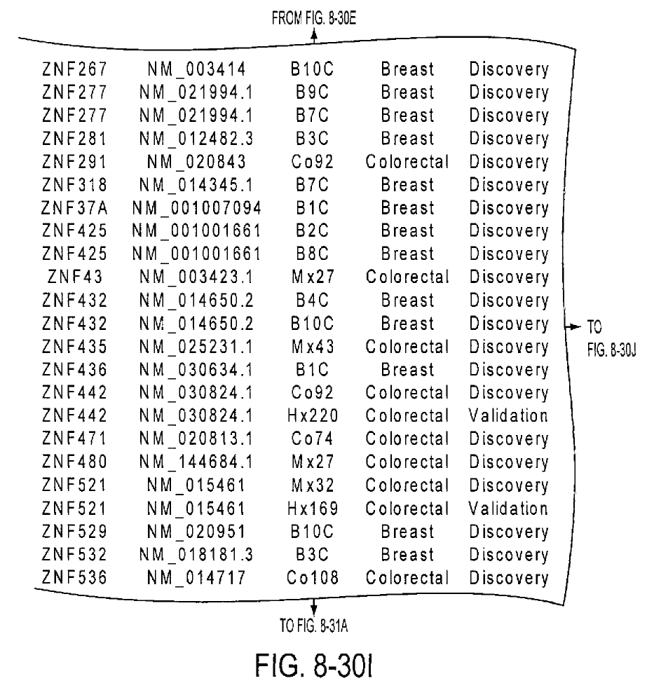

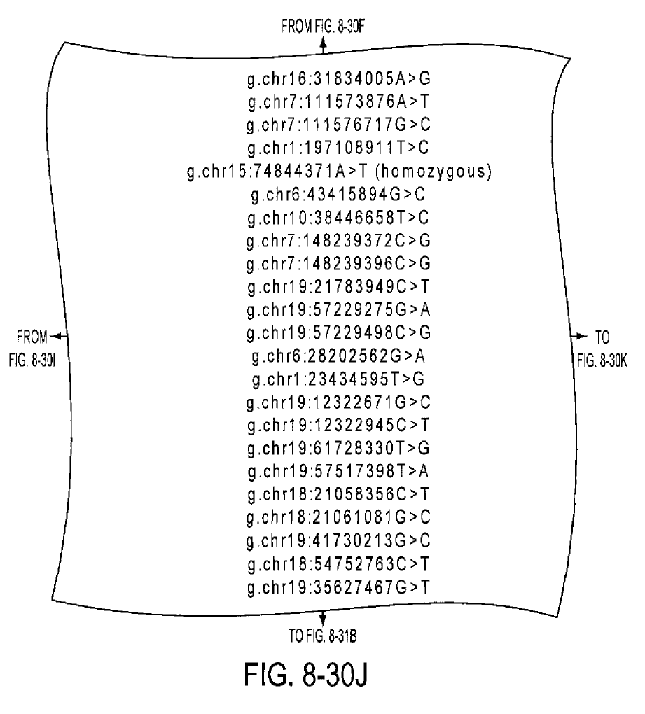

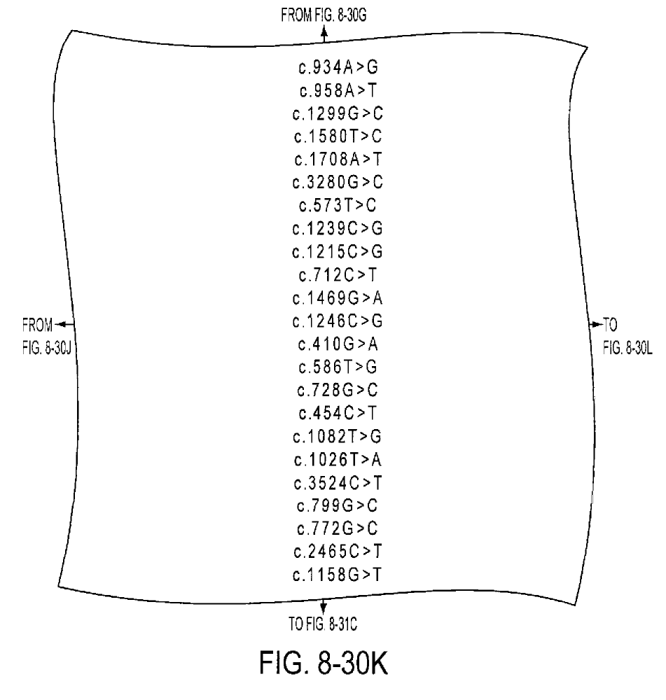

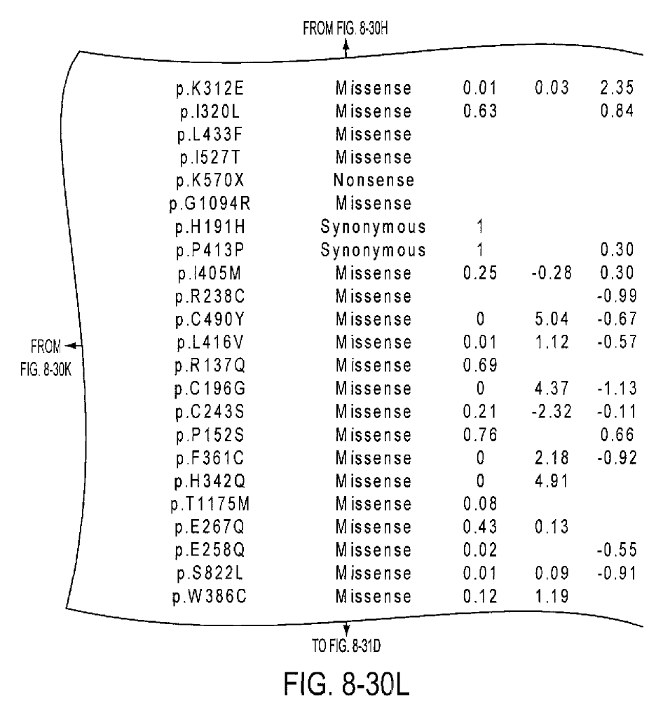

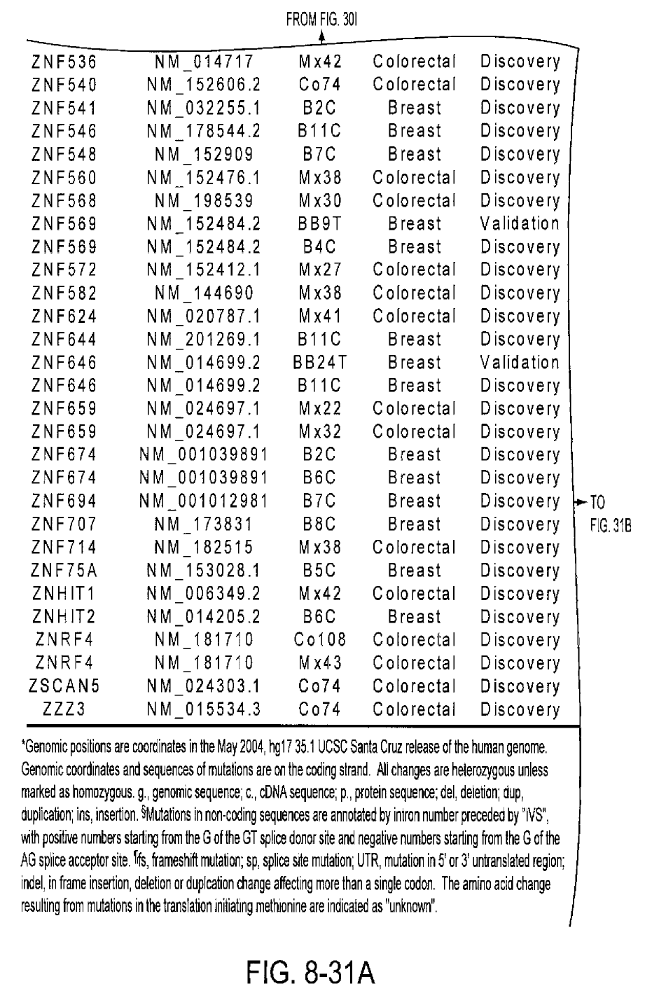

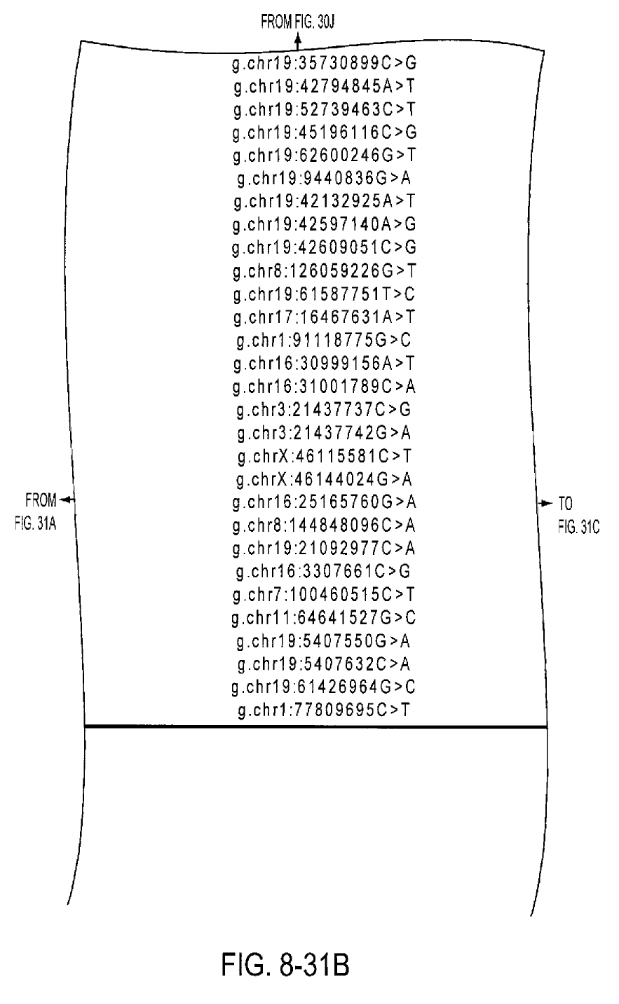

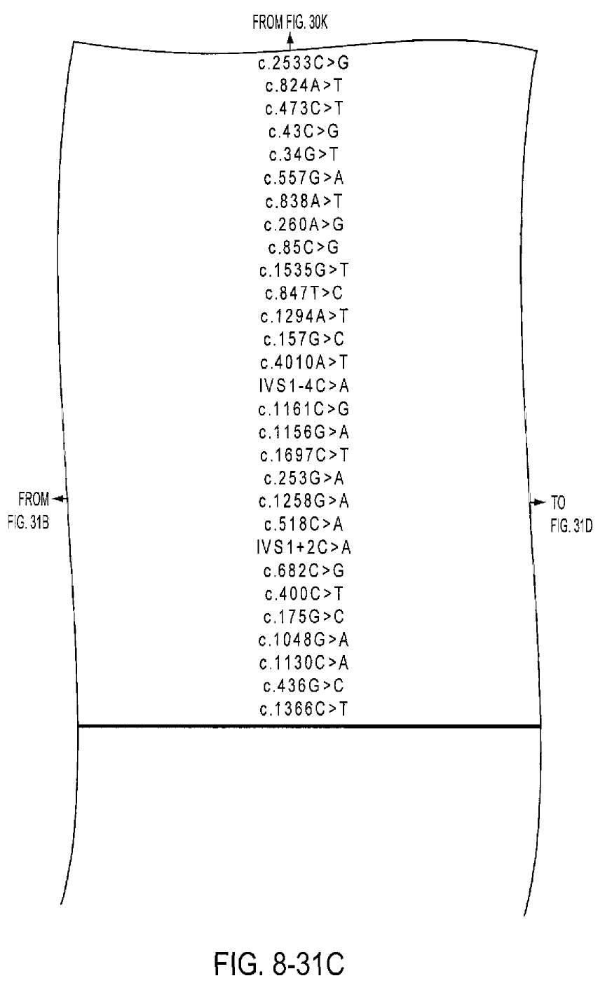

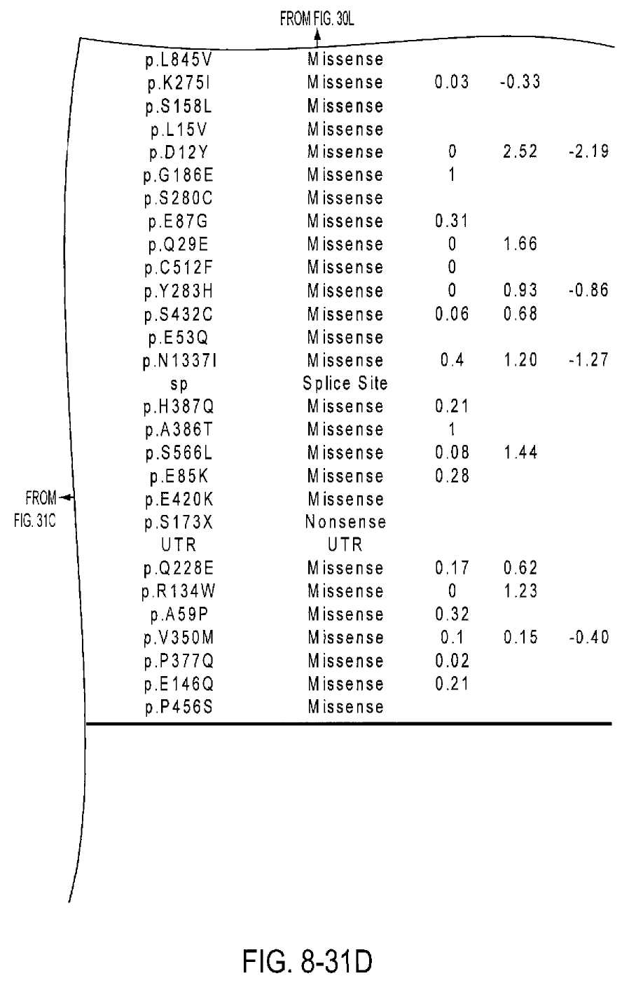

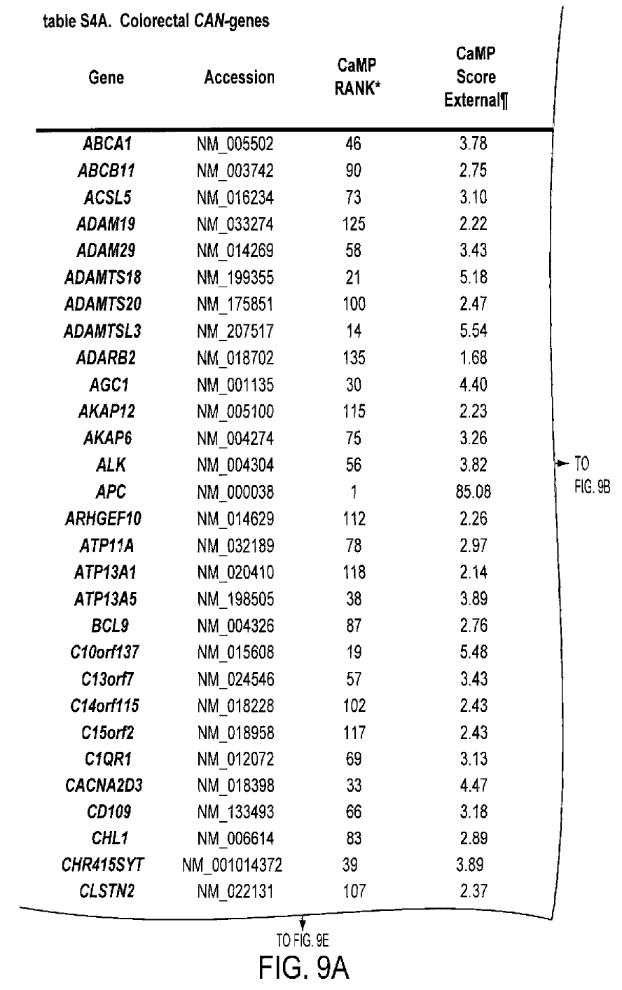

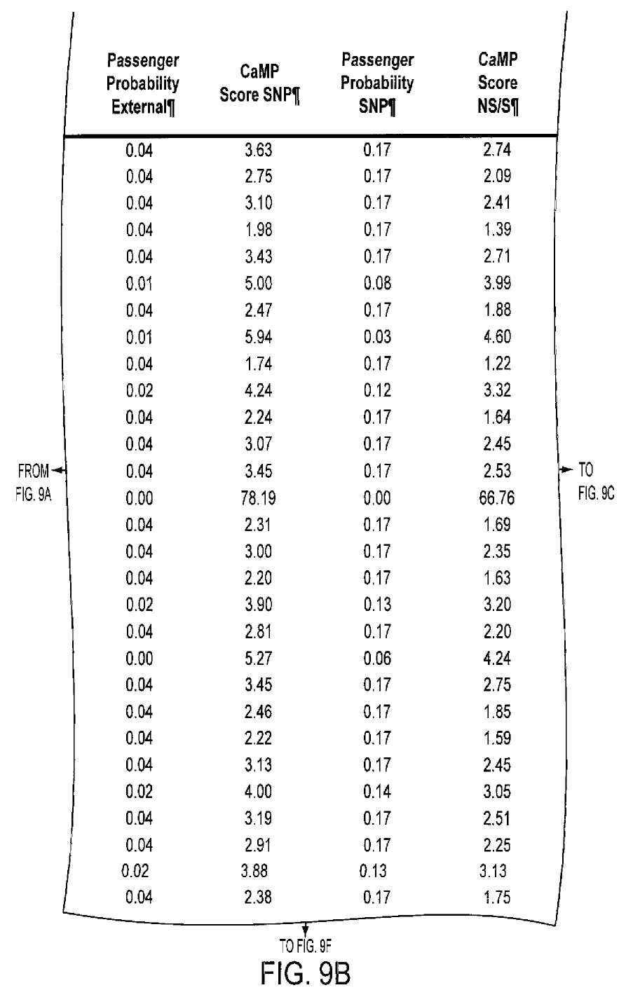

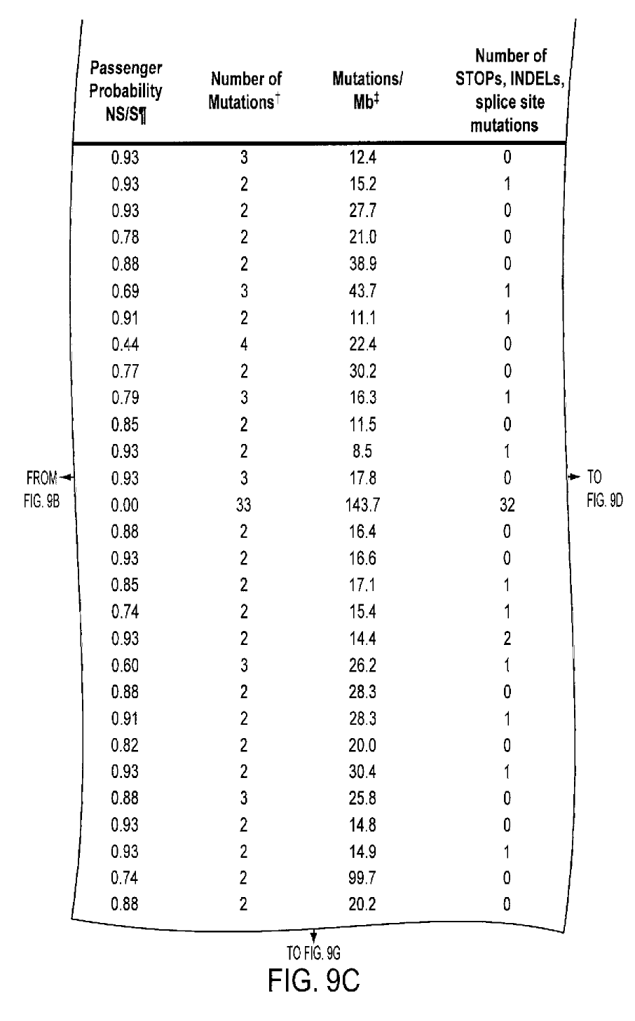

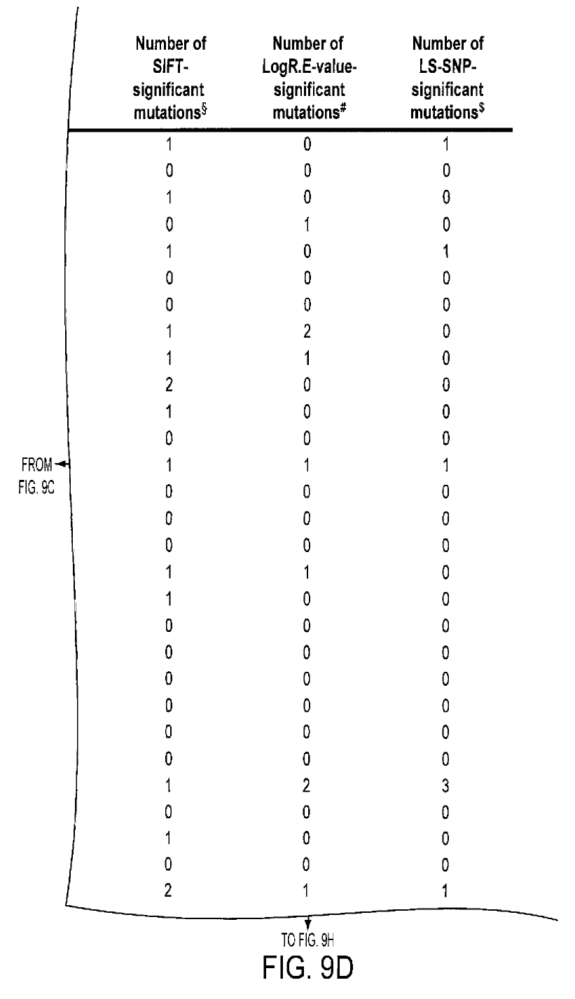

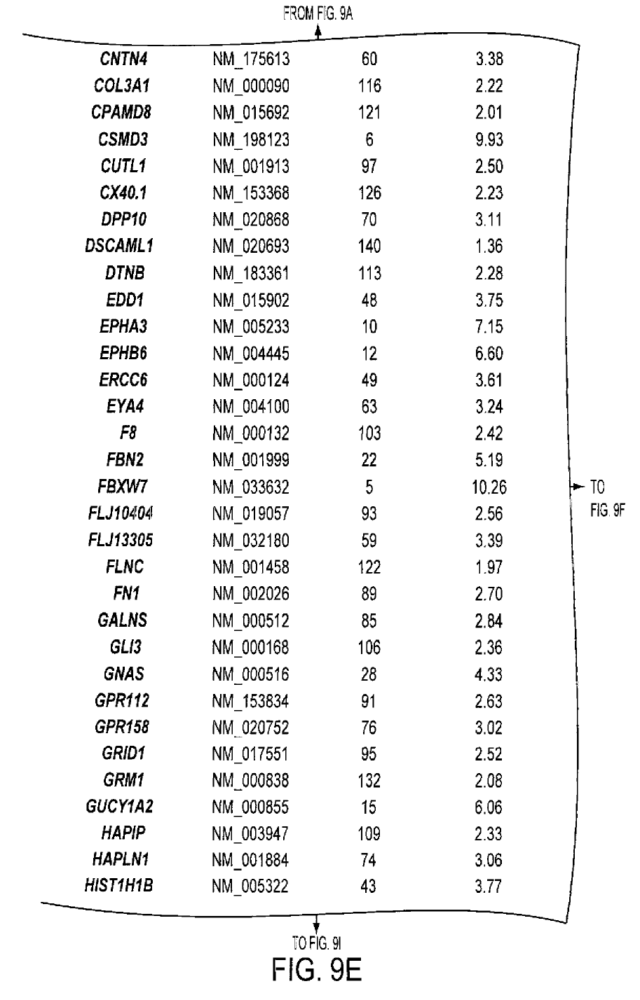

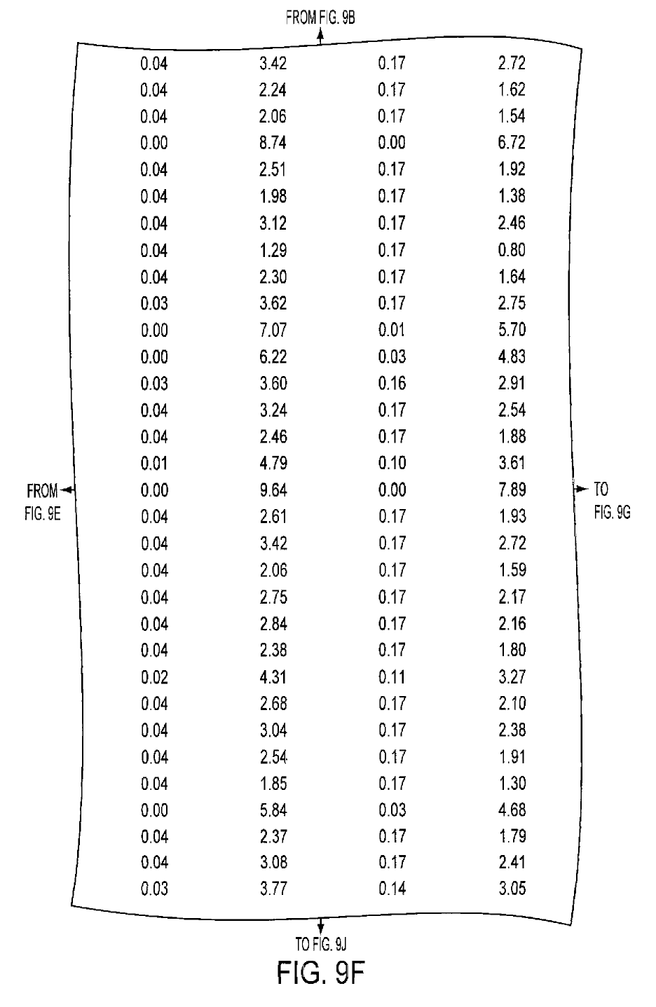

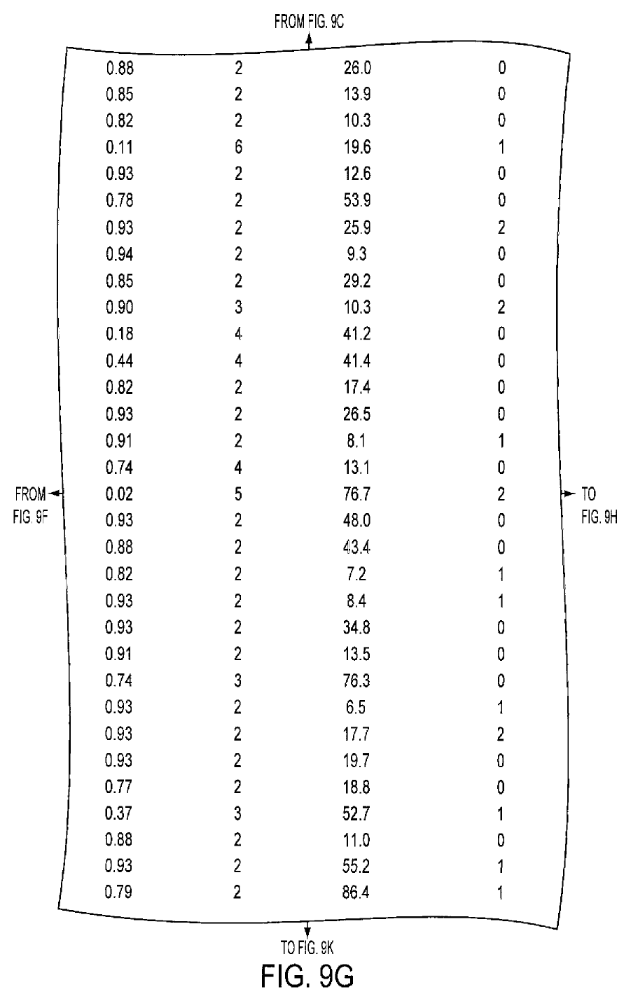

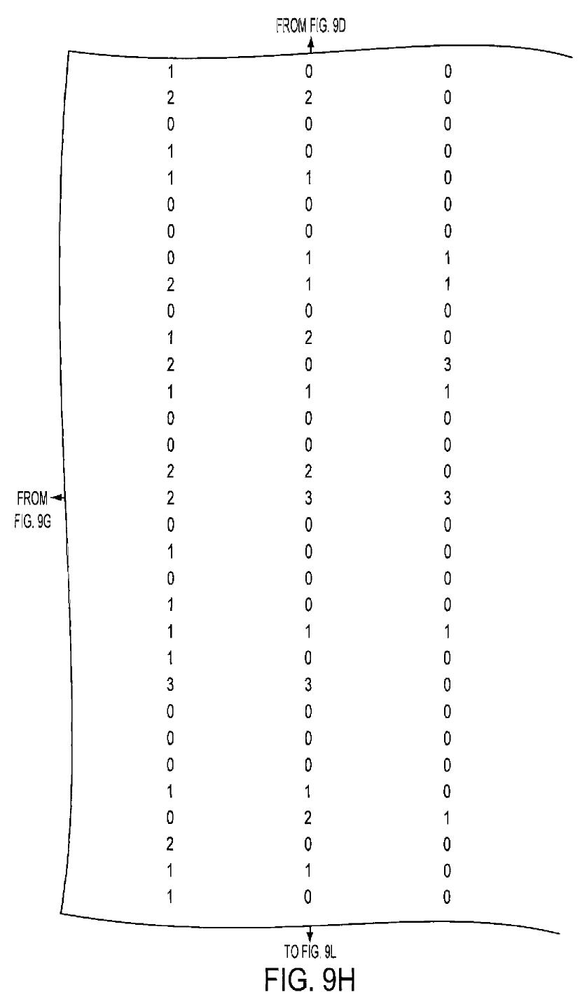

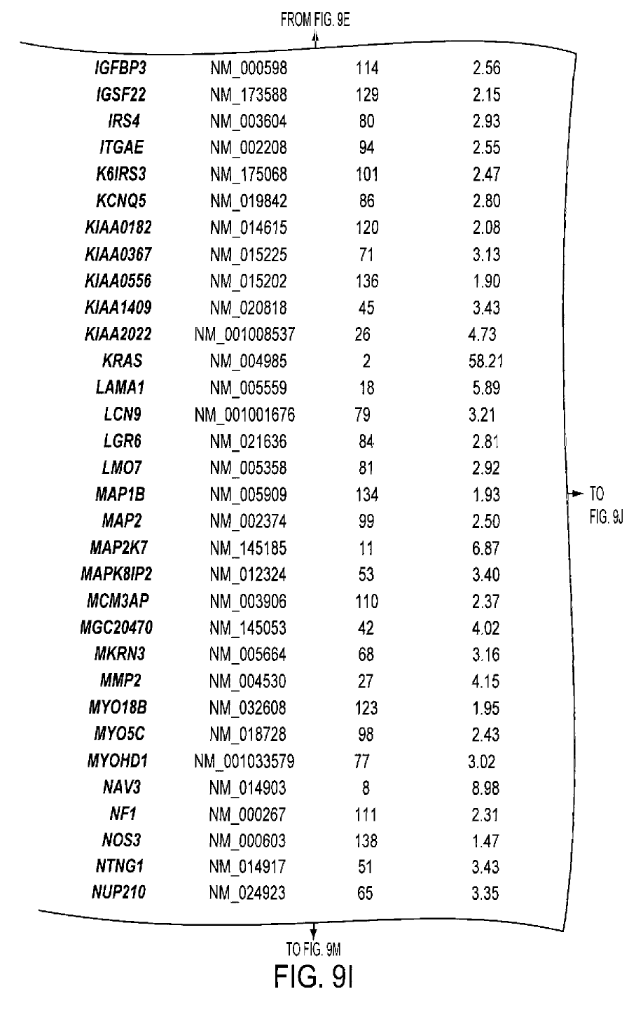

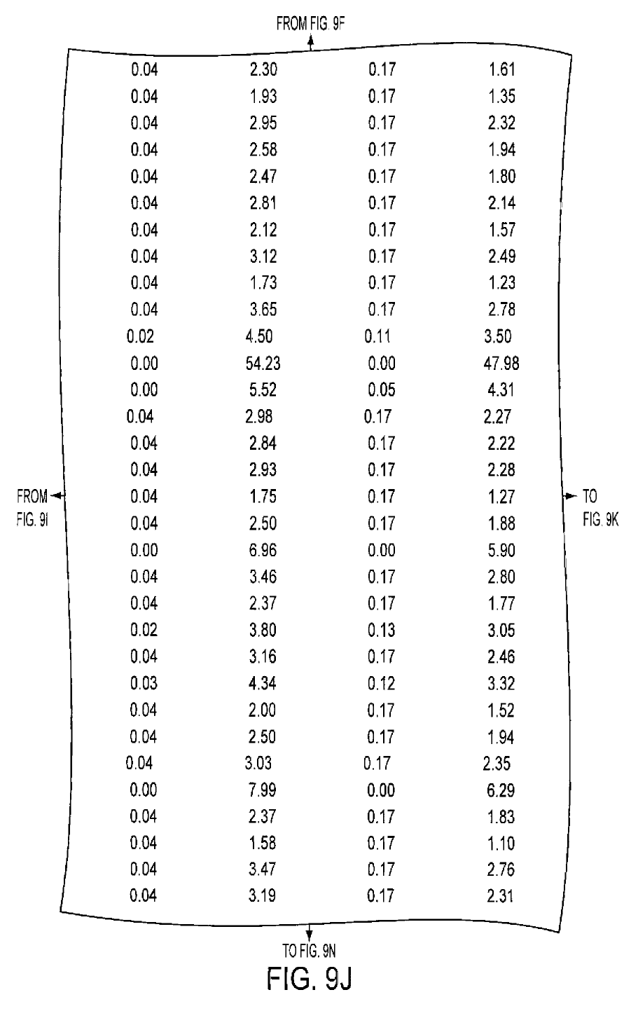

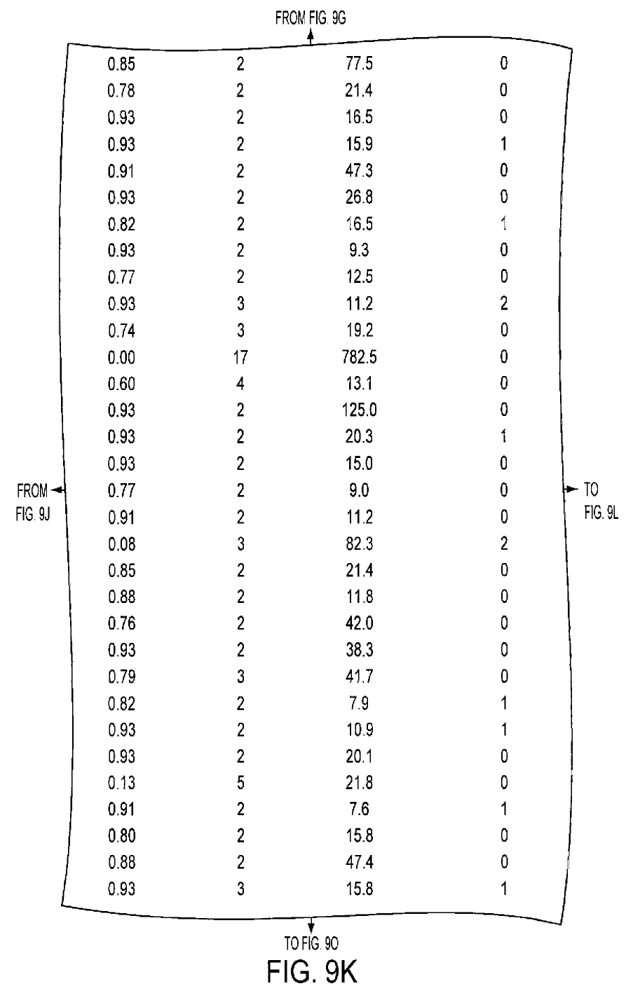

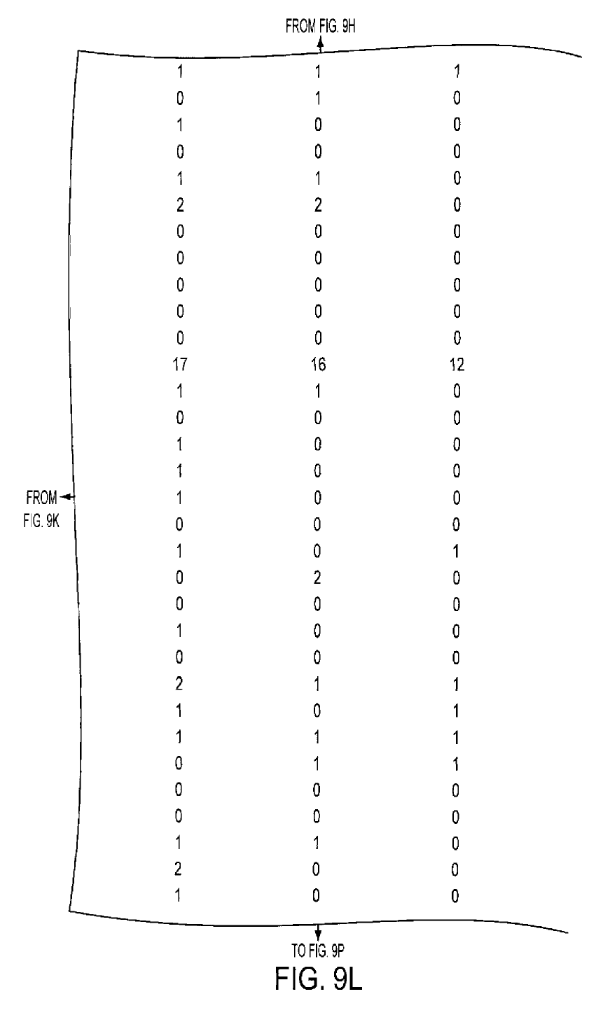

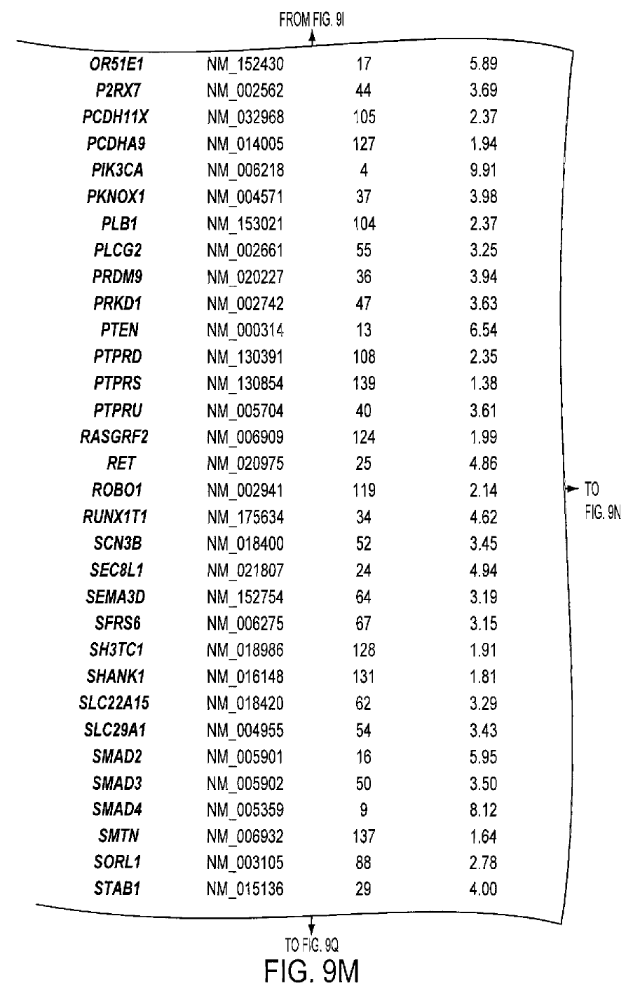

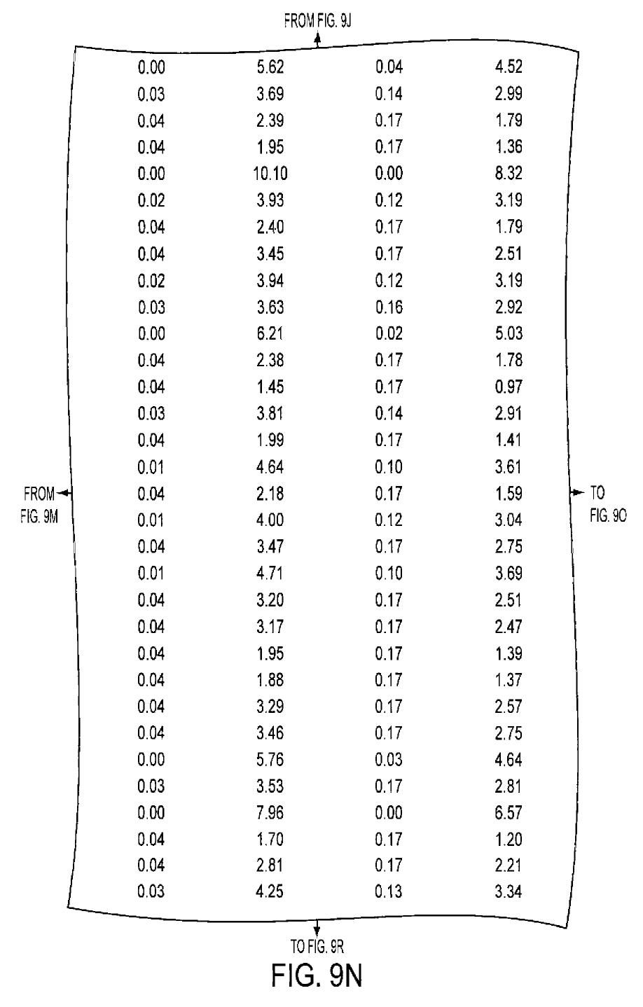

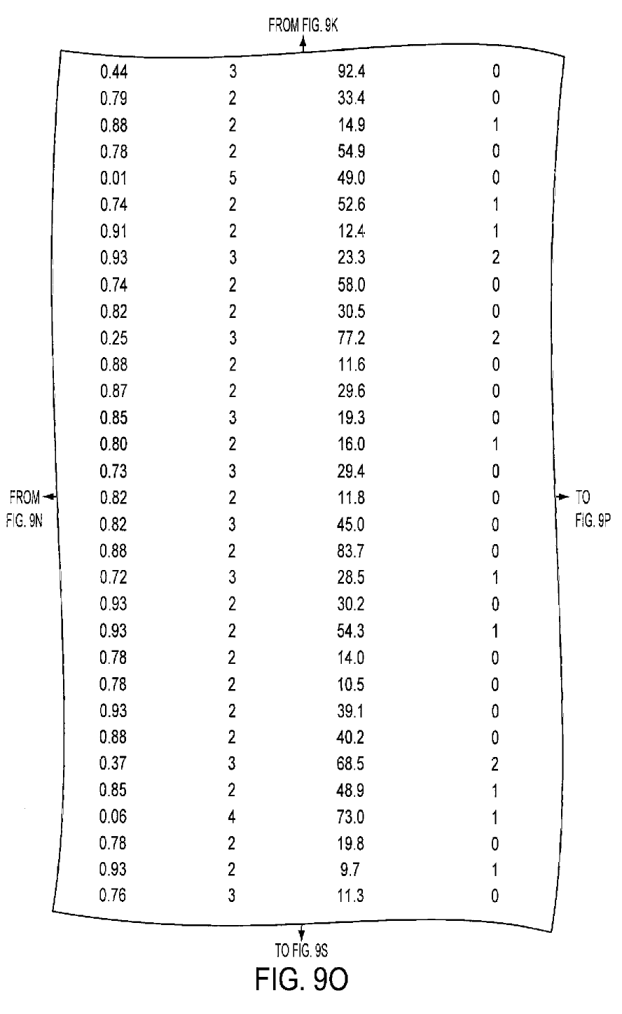

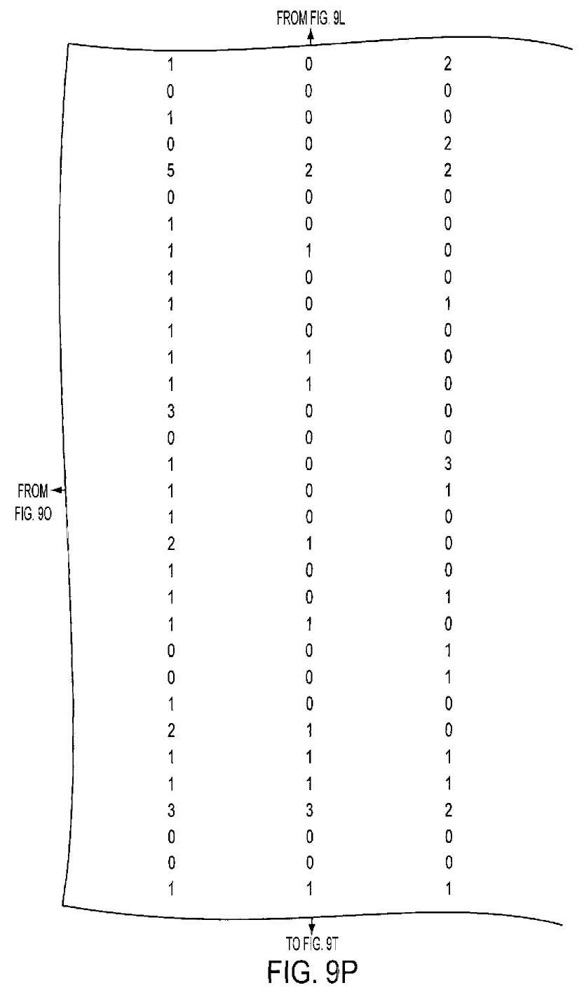

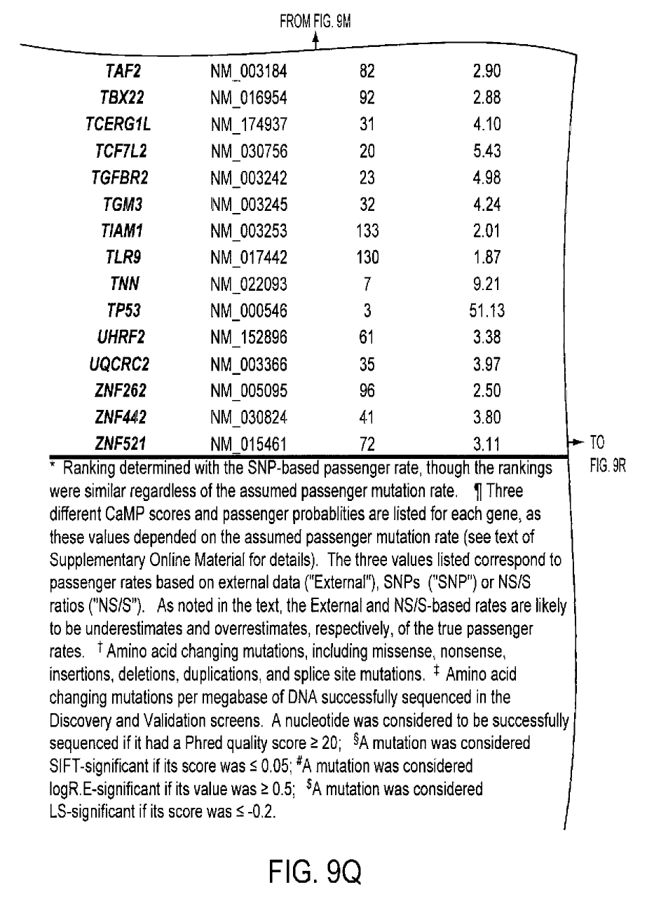

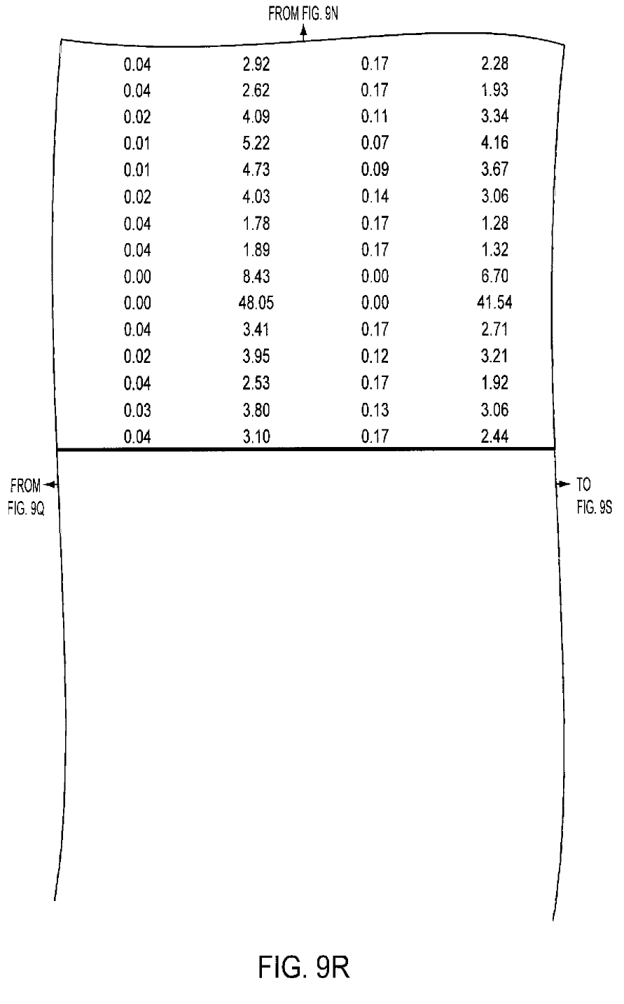

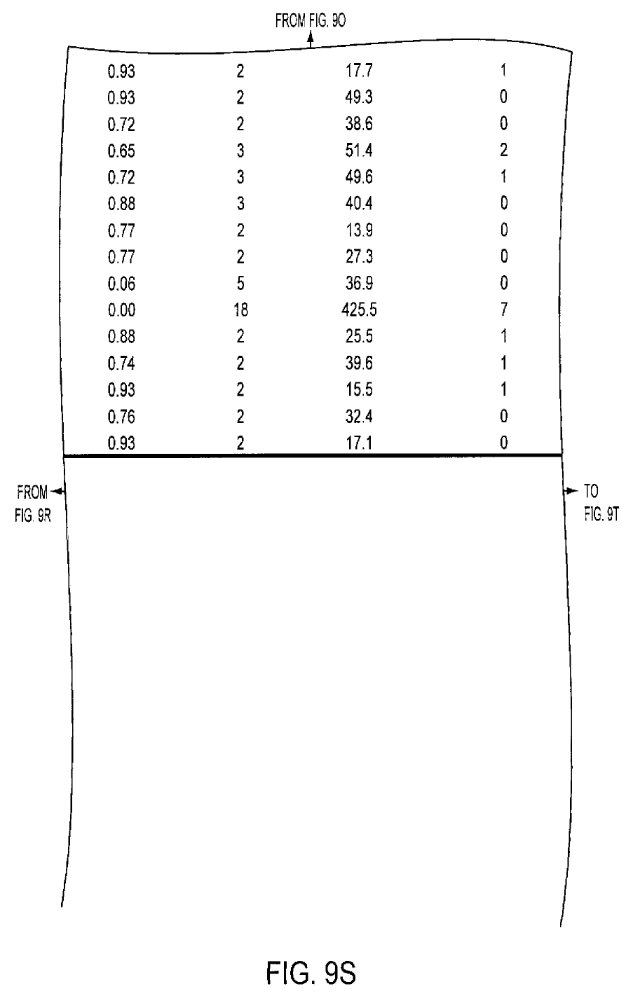

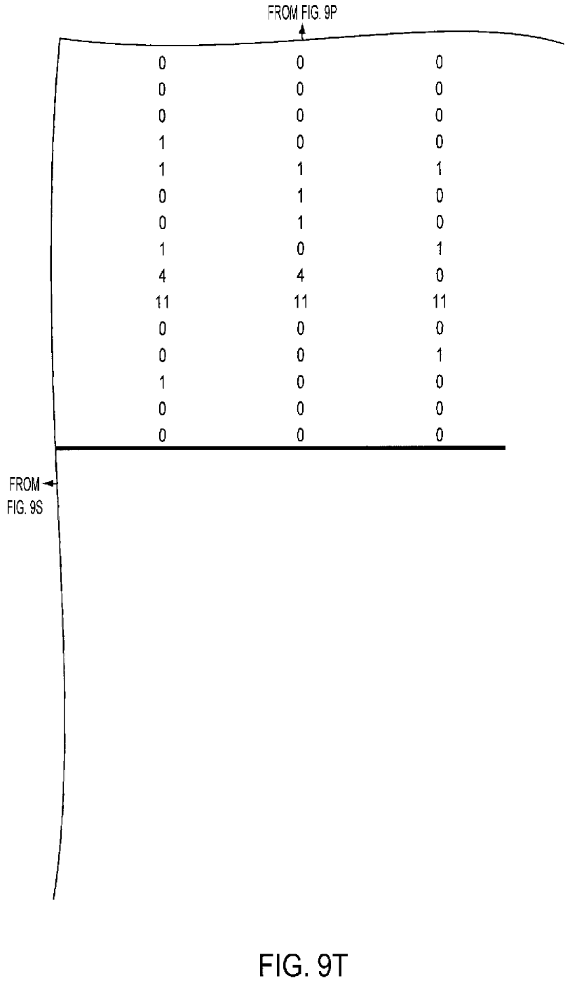

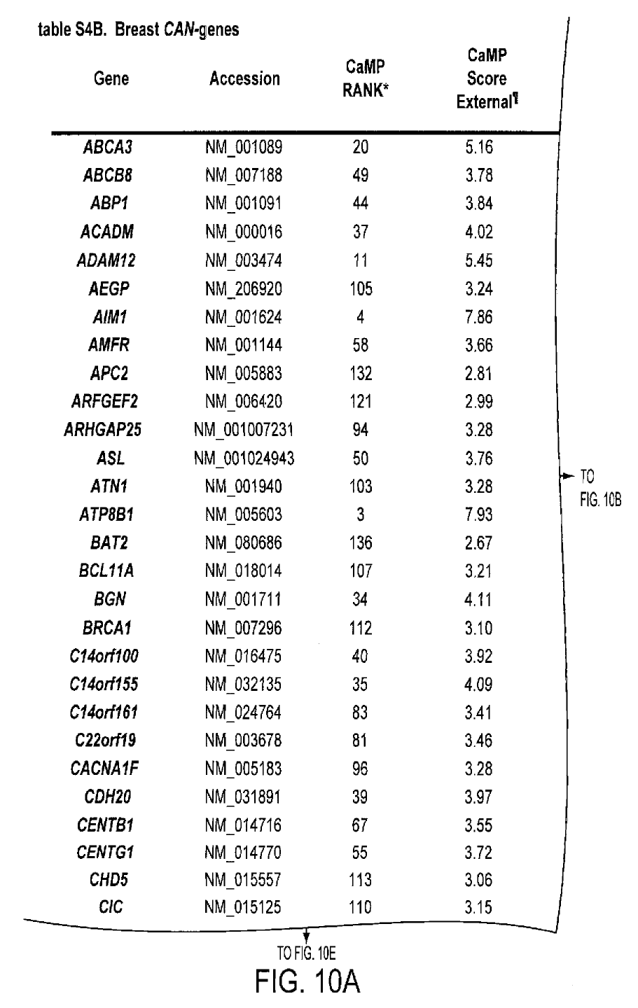

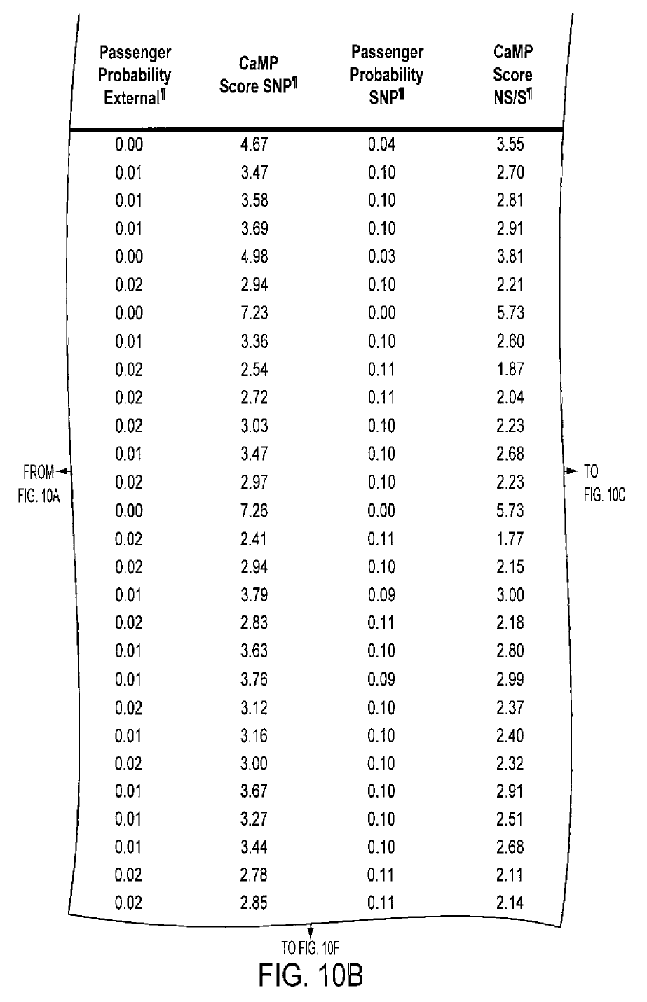

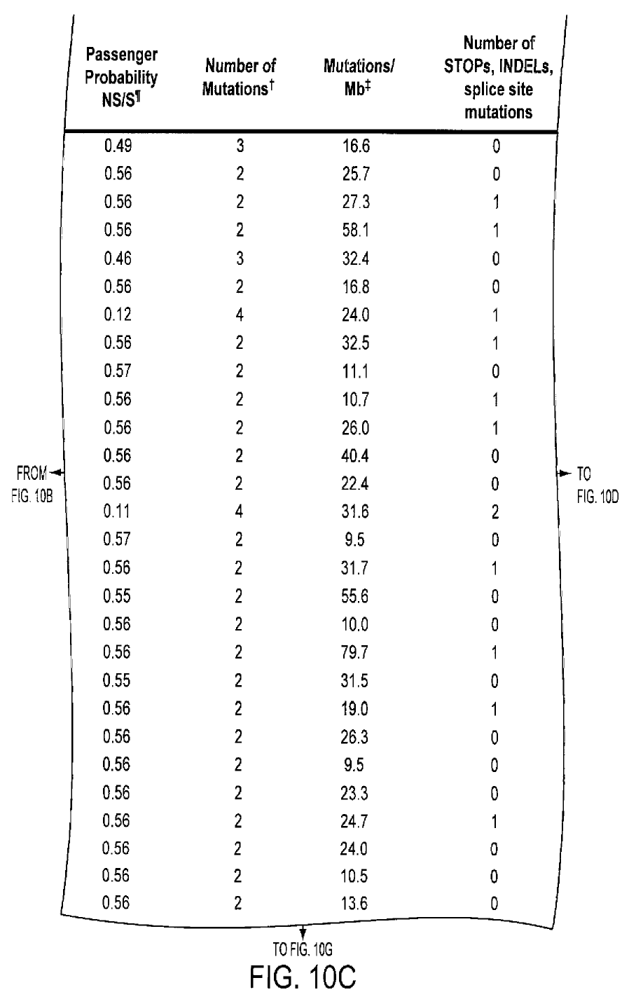

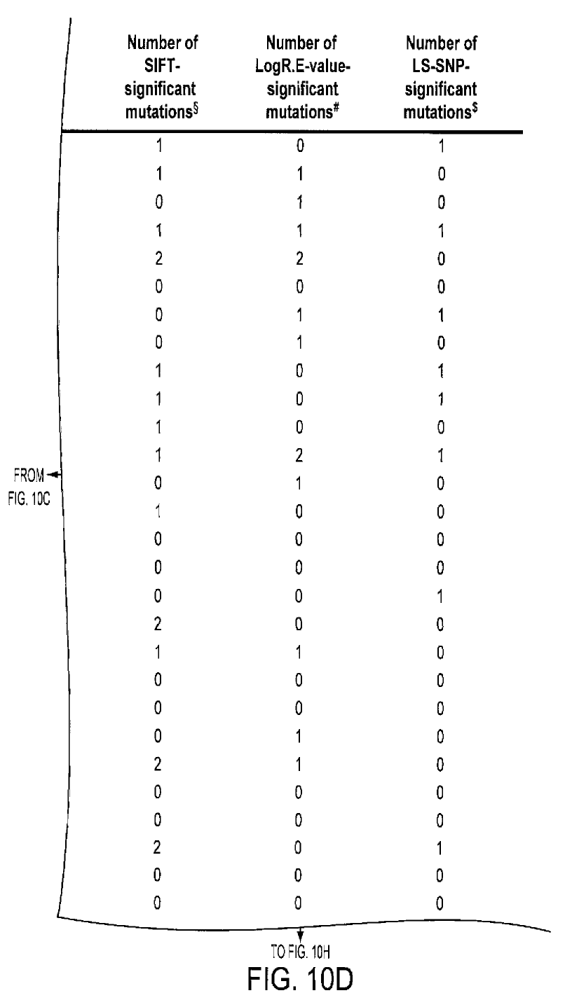

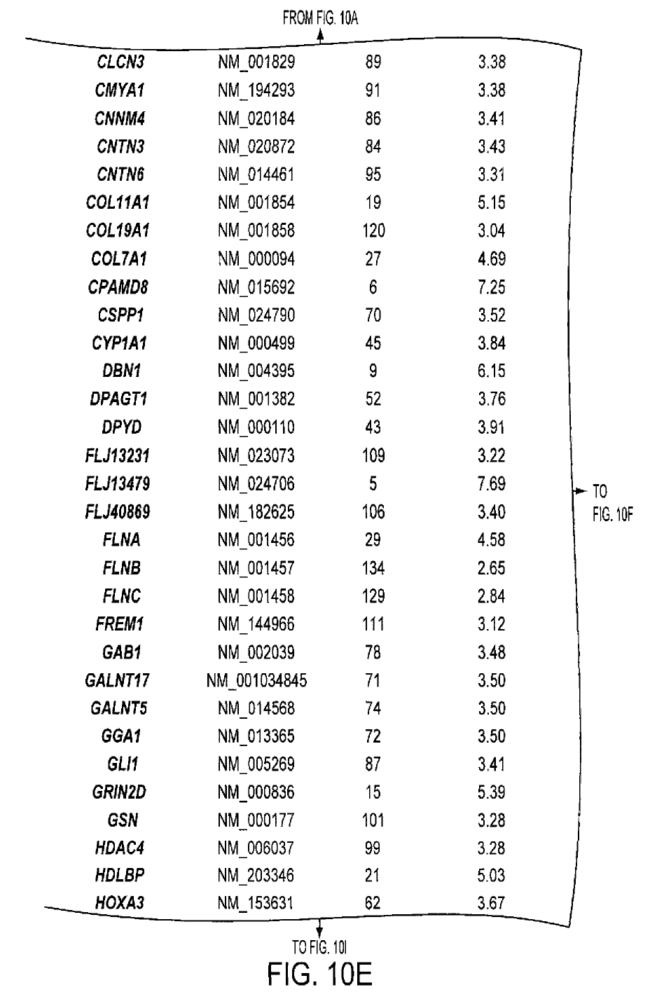

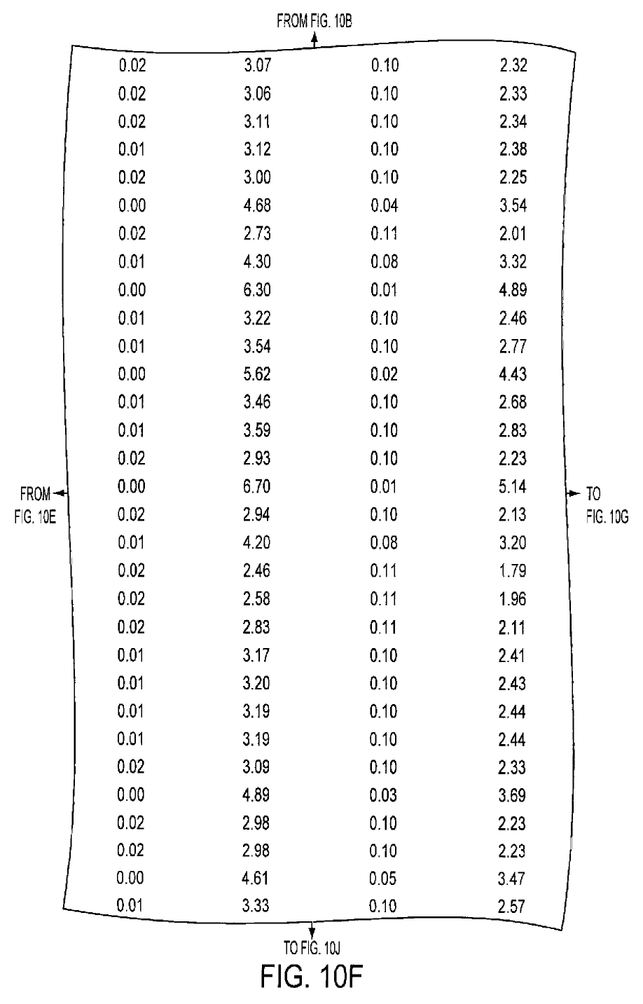

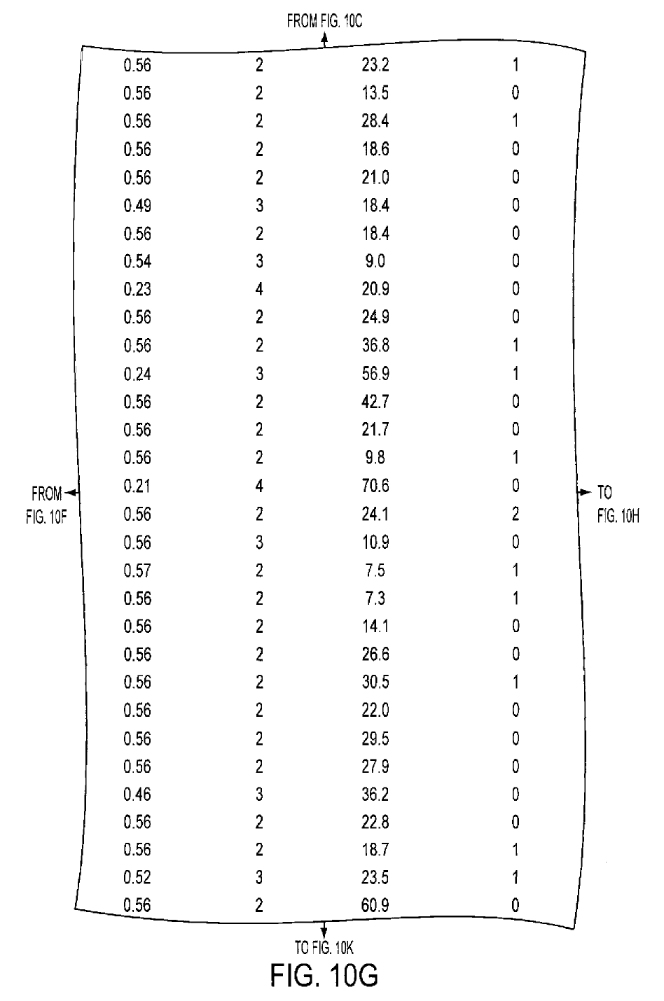

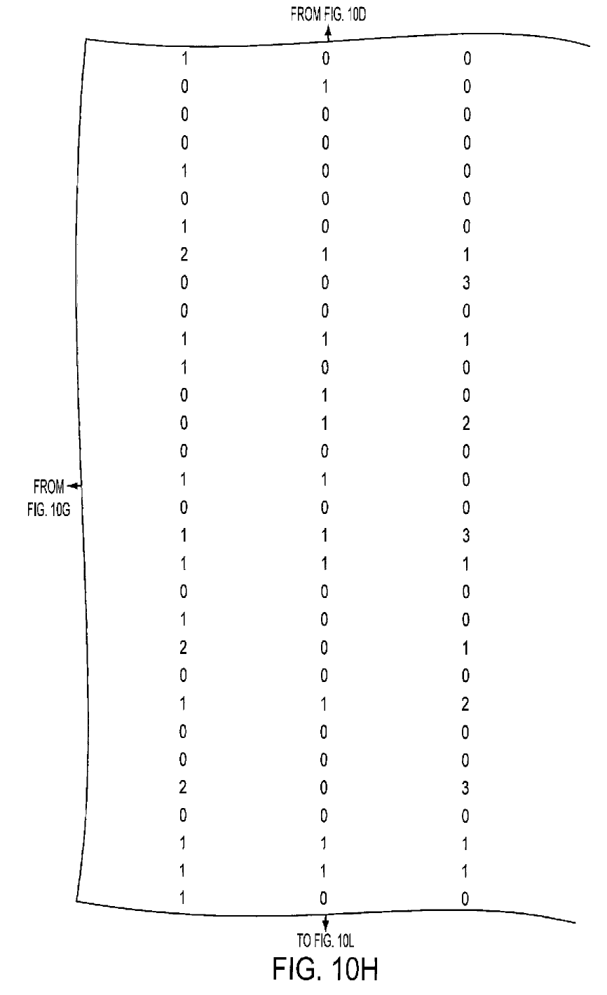

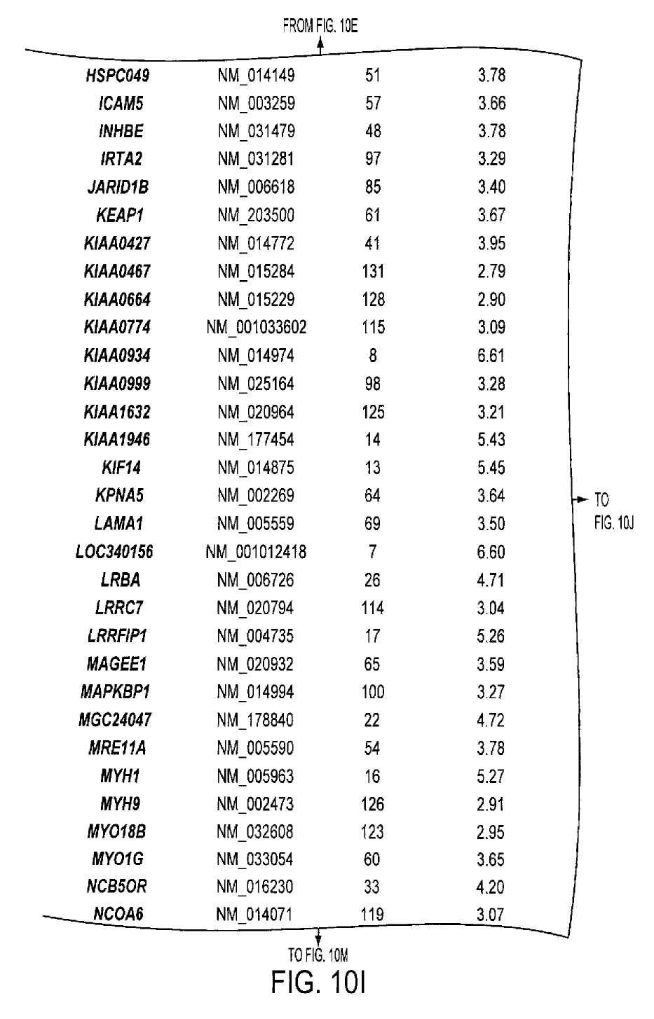

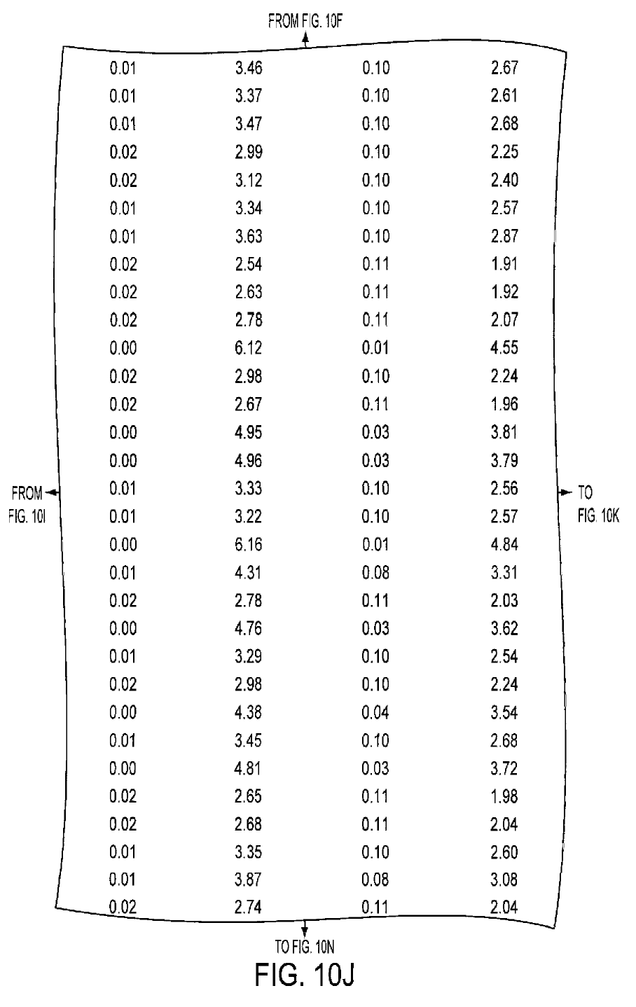

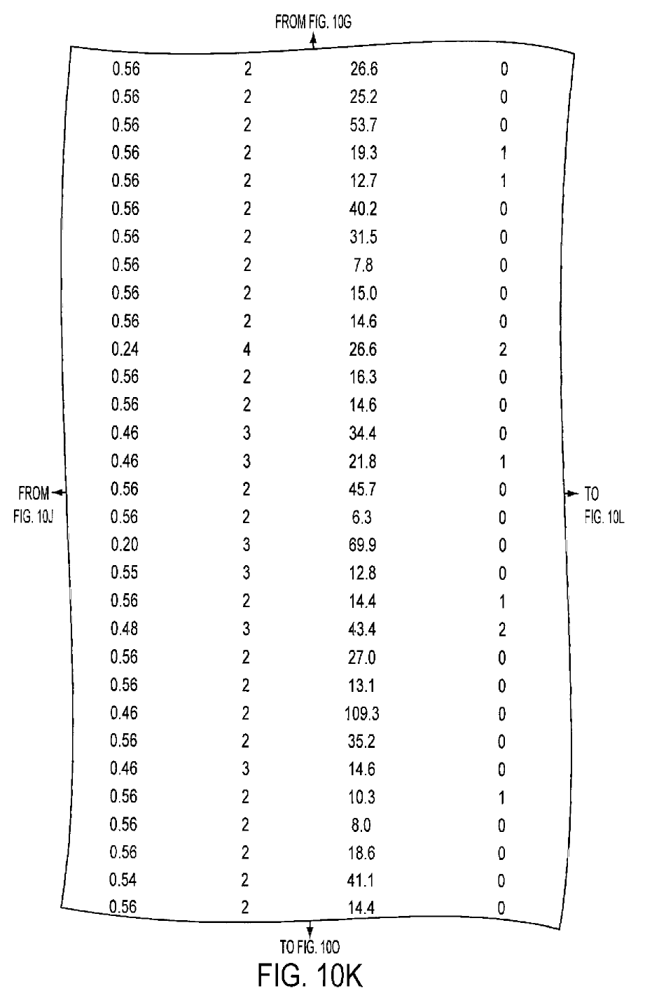

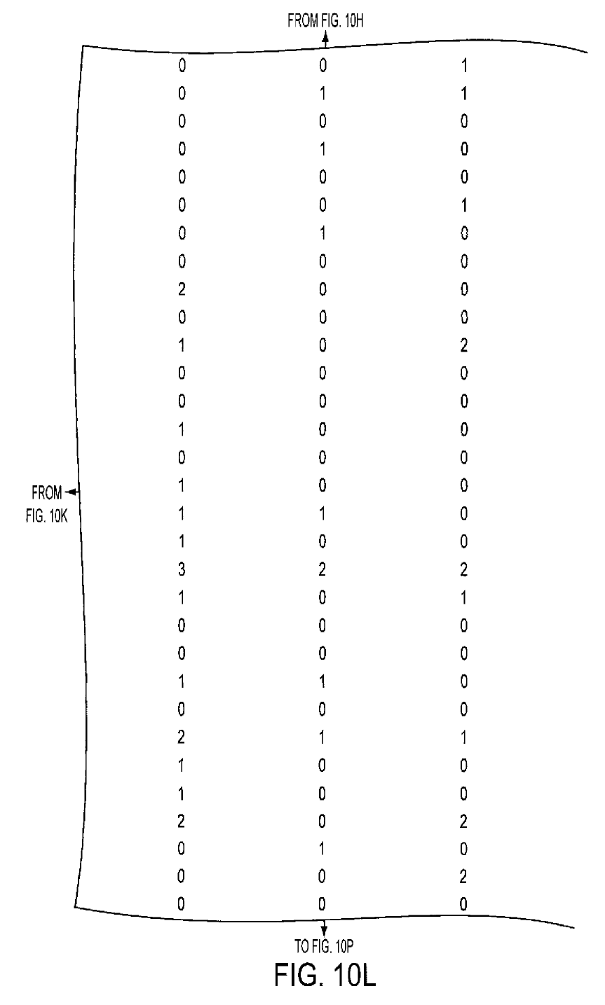

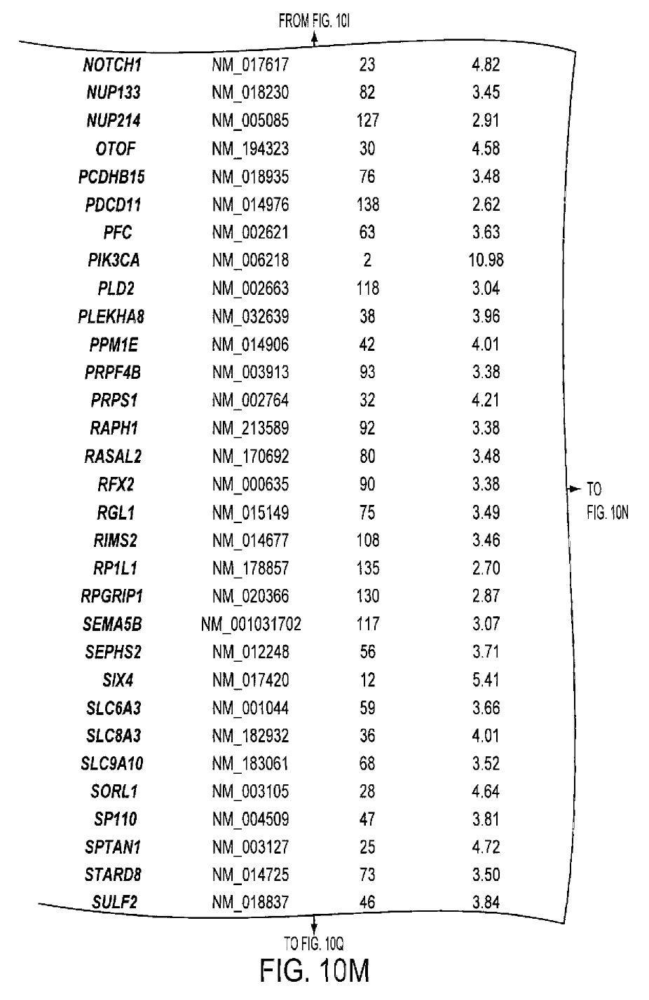

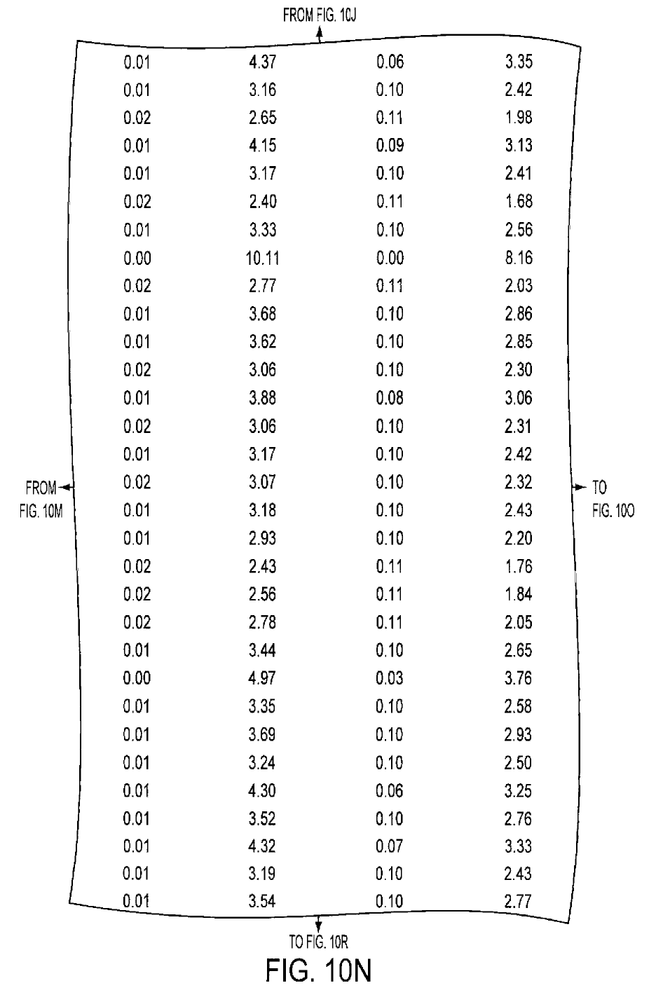

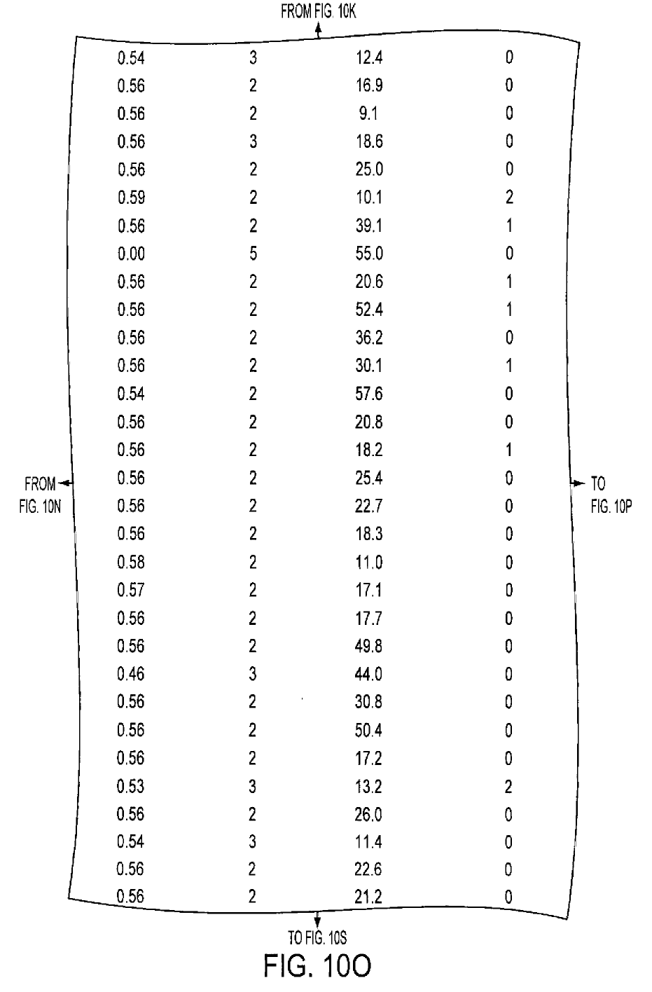

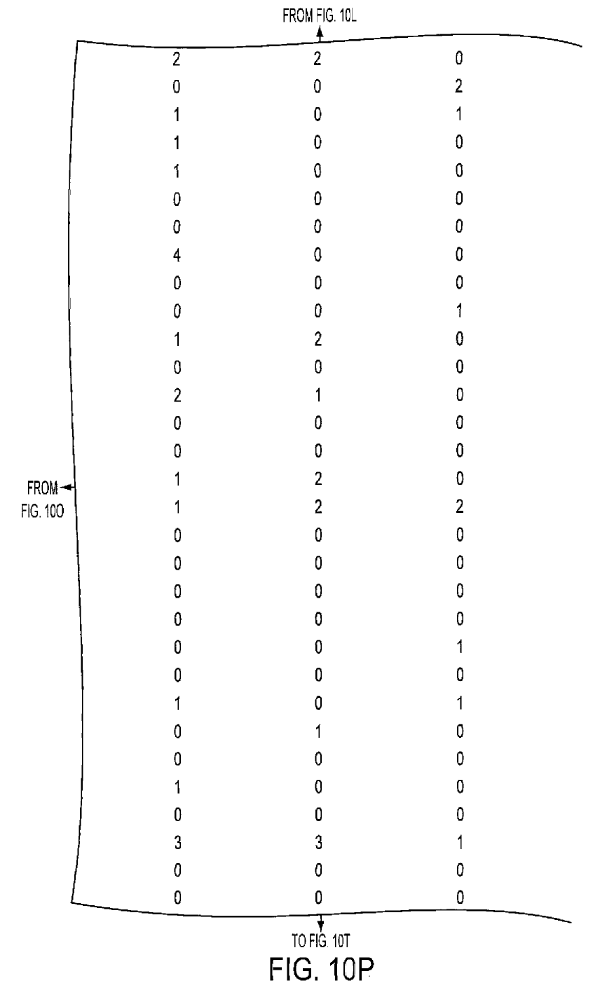

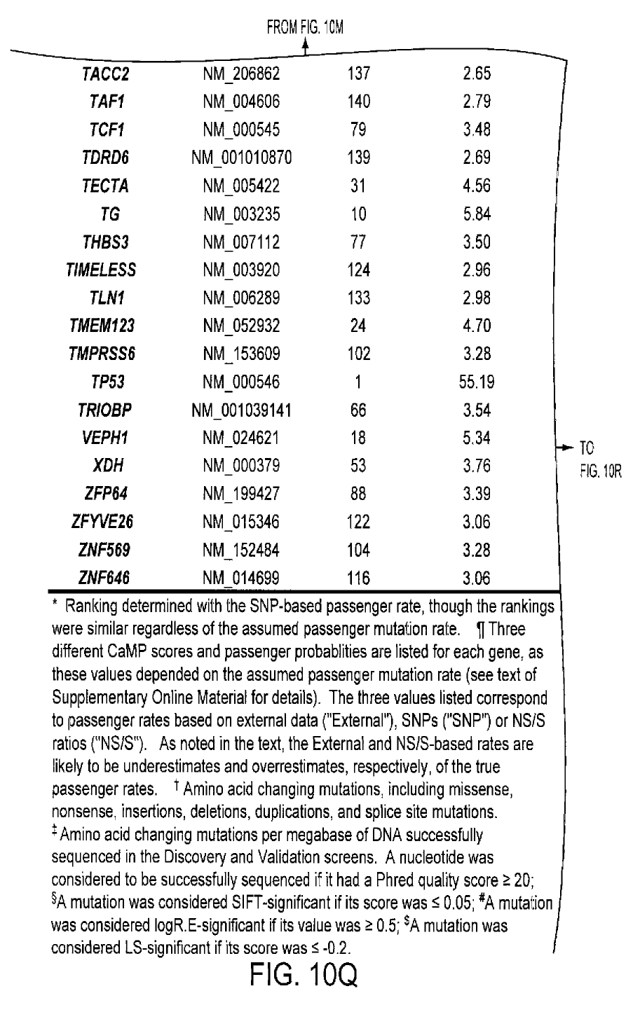

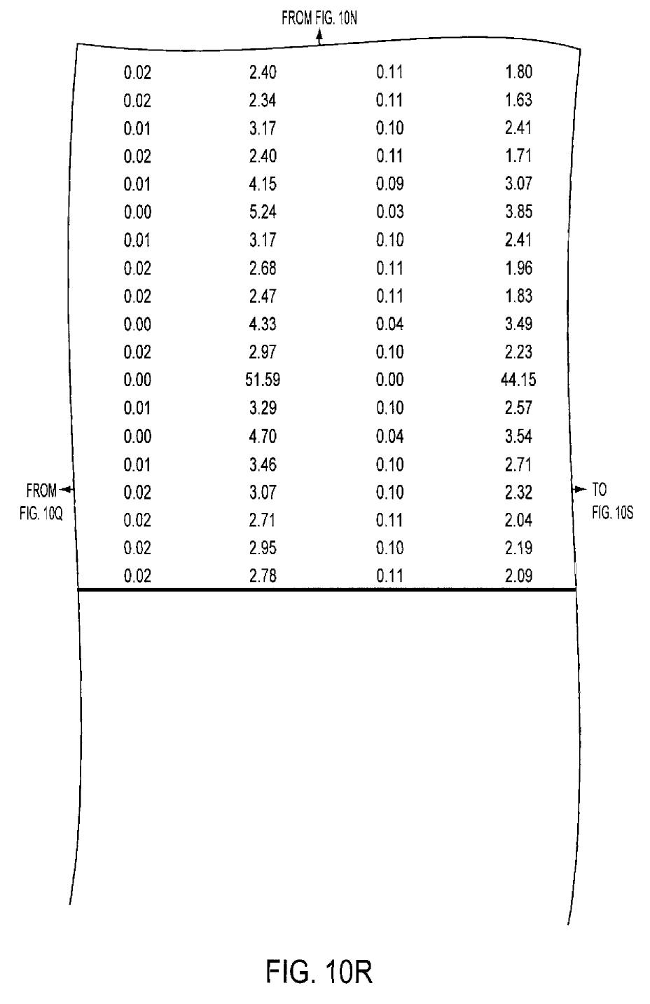

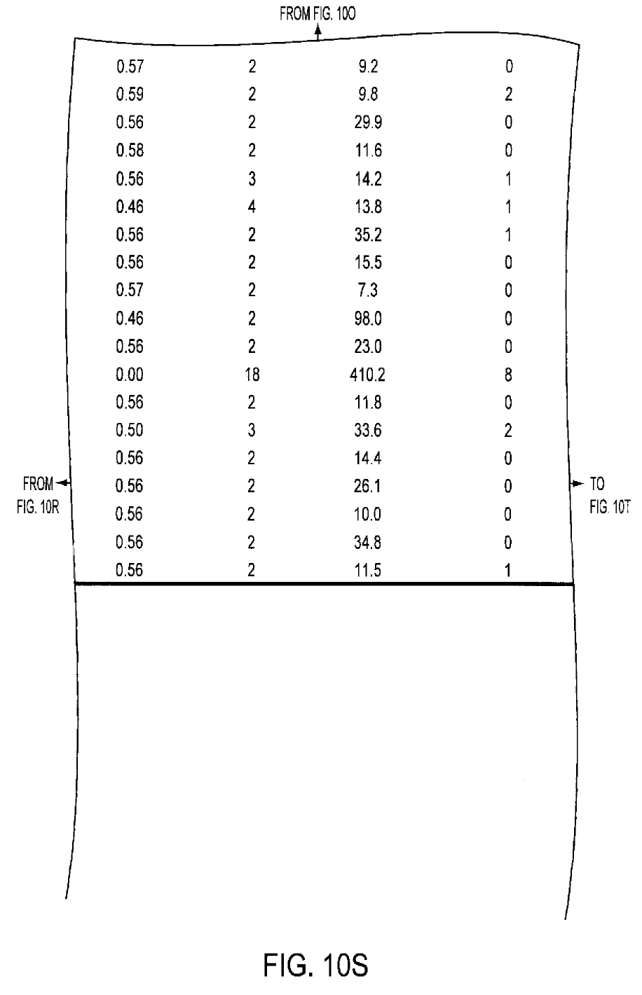

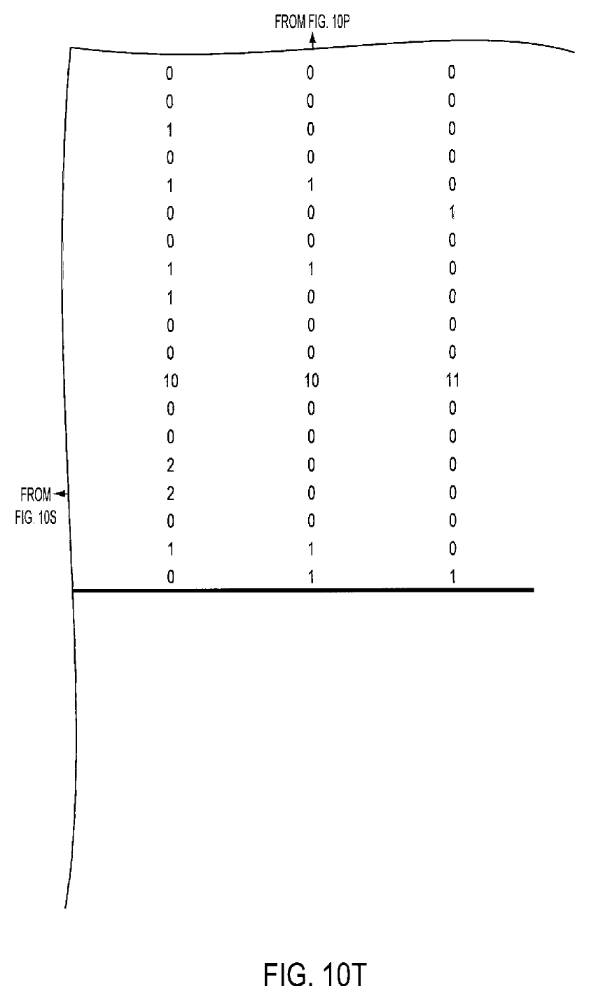

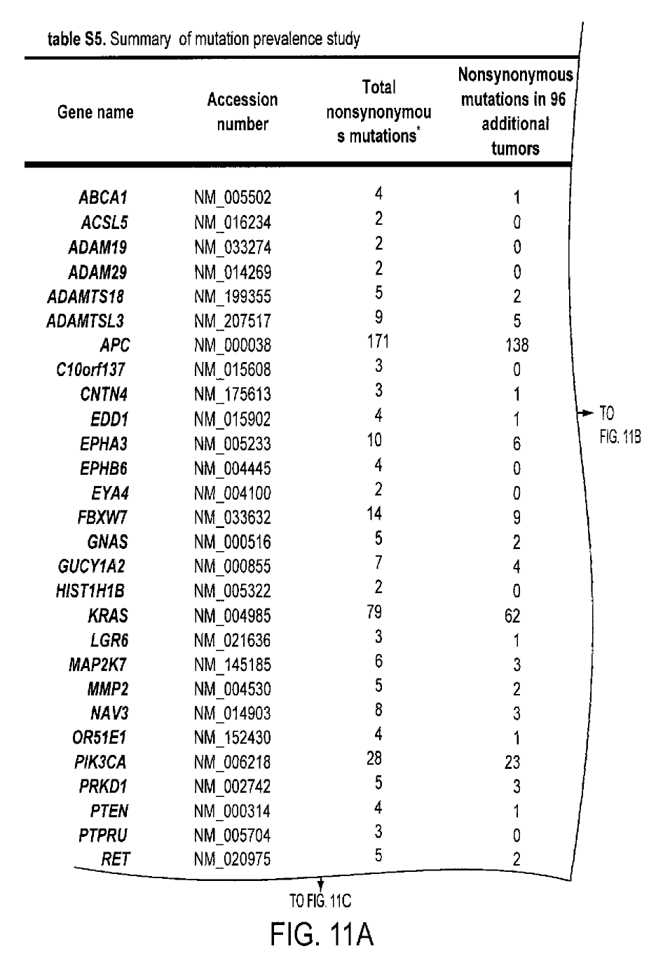

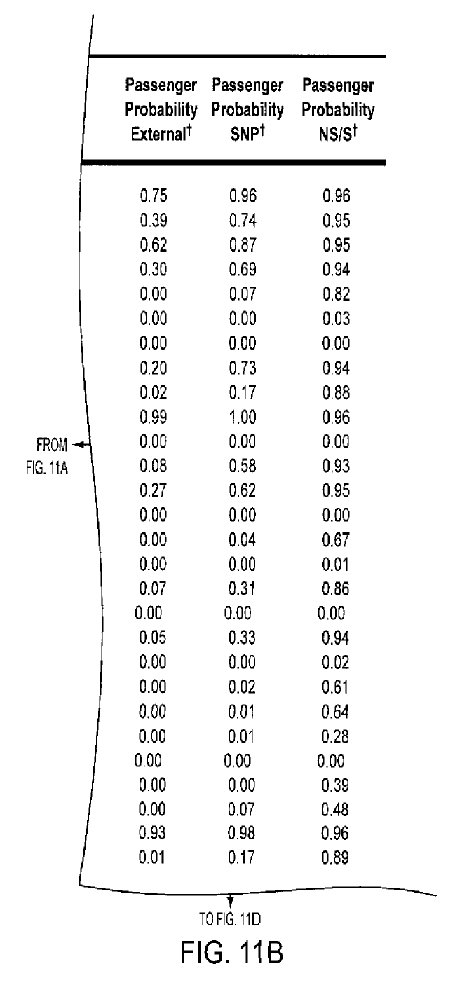

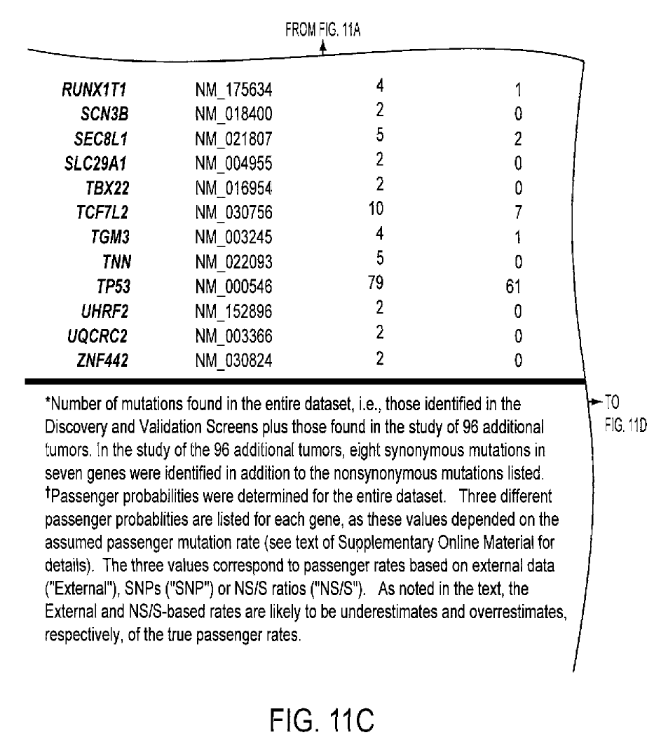

Somatic mutations are determined by finding a difference between a test sample and a normal sample of a human. This criterion eliminates the possibility of germ-line differences confounding the analysis. For breast cancer, the gene (or cDNA or protein) to be tested is any of those shown in FIG. 10. Table S4B. Any somatic mutation may be informative. Particular mutations which may be used are shown in FIG. 8 (Table S3). For colon cancer, the gene (or cDNA or protein) to be tested is any of those shown in FIG. 9. Table S4A. Any somatic mutation may be informative. Particular mutations which may be used are shown in FIG. 8 (Table S3).

The number of genes or mutations that may be useful in forming a signature of a breast or colorectal cancer may vary from one to twenty-five. At least two, three, four, five, six, seven, ten, fifteen, twenty, or more genes may be used. The mutations are typically somatic mutations and non-synonymous mutations. Those mutations described here are within coding regions. Other non-coding region mutations may also be found and may be informative.

In order to test candidate or already-identified therapeutic agents to determine which patients and tumors will be sensitive to the agents, stratification on the basis of signatures can be used. One or more groups with a similar mutation signature will be formed and the effect of the therapeutic agent on the group will be compared to the effect of patients whose tumors do not share the signature of the group formed. The group of patients who do not share the signature may share a different signature or they may be a mixed population of tumor-bearing patients whose tumors bear a variety of signatures.

Efficacy can be determined by any of the standard means known in the art. Any index of efficacy can be used. The index may be life span, disease free remission period, tumor shrinkage, tumor growth arrest, improvement of quality of life, decreased side effects, decreased pain, etc. Any useful measure of patient health and well-being can be used. In addition, in vitro testing may be done on tumor cells that have particular signatures. Tumor cells with particular signatures can also be tested in animal models.

Once a signature has been correlated with sensitivity or resistance to a particular therapeutic regimen, that signature can be used for prescribing a treatment to a patient. Thus determining a signature is useful for making therapeutic decisions. The signature can also be combined with other physical or biochemical findings regarding the patient to arrive at a therapeutic decision. A signature need not be the sole basis for making a therapeutic decision.

An anti-cancer agent associated with a signature may be, for example, docetaxel, paclitaxel, topotecan, adriamycin, etoposide, fluorouracil (5-FU), or cyclophosphamide. The agent may be an alkylating agent (e.g., nitrogen mustards), antimetabolites (e.g., pyrimidine analogs), radioactive isotopes (e.g., phosphorous and iodine), miscellaneous agents (e.g., substituted ureas) and natural products (e.g., vinca alkyloids and antibiotics). The therapeutic agent may be allopurinol sodium, dolasetron mesylate, pamidronate disodium, etidronate, fluconazole, epoetin alfa, levamisole HCL, amifostine, granisetron HCL, leucovorin calcium, sargramostim, dronabinol, mesna, filgrastim, pilocarpine HCL, octreotide acetate, dexrazoxane, ondansetron HCL, ondansetron, busulfan, carboplatin, cisplatin, thiotepa, melphalan HCL, melphalan, cyclophosphamide, ifosfamide, chlorambucil, mechlorethamine HCL, carmustine, lomustine, polifeprosan 20 with carmustine implant, streptozocin, doxorubicin HCL, bleomycin sulfate, daunirubicin HCL, dactinomycin, daunorucbicin citrate, idarubicin HCL, plimycin, mitomycin, pentostatin, mitoxantrone, valrubicin, cytarabine, fludarabine phosphate, floxuridine, cladribine, methotrexate, mercaptipurine, thioguanine, capecitabine, methyltestosterone, nilutamide, testolactone, bicalutamide, flutamide, anastrozole, toremifene citrate, estramustine phosphate sodium, ethinyl estradiol, estradiol, esterified estrogens, conjugated estrogens, leuprolide acetate, goserelin acetate, medroxyprogesterone acetate, megestrol acetate, levamisole HCL, aldesleukin, irinotecan HCL, dacarbazine, asparaginase, etoposide phosphate, gemcitabine HCL, altretamine, topotecan HCL, hydroxyurea, interferon alpha-2b, mitotane, procarbazine HCL, vinorelbine tartrate, E. coli L-asparaginase, Erwinia L-asparaginase, vincristine sulfate, denileukin diftitox, aldesleukin, rituximab, interferon alpha-2a, paclitaxel, docetaxel, BCG live (intravesical), vinblastine sulfate, etoposide, tretinoin, teniposide, porfimer sodium, fluorouracil, betamethasone sodium phosphate and betamethasone acetate, letrozole, etoposide citrororum factor, folinic acid, calcium leucouorin, 5-fluorouricil, adriamycin, cytoxan, or diamino-dichloro-platinum.

The signatures of CAN genes according to the present invention can be used to determine an appropriate therapy for an individual. For example, a sample of a tumor (e.g., a tissue obtained by a biopsy procedure, such as a needle biopsy) can be provided from the individual, such as before a primary therapy is administered. The gene expression profile of the tumor can be determined, such as by a nucleic acid array (or protein array) technology, and the expression profile can be compared to a database correlating signatures with treatment outcomes. Other information relating to the human (e.g., age, gender, family history, etc.) can factor into a treatment recommendation. A healthcare provider can make a decision to administer or prescribe a particular drug based on the comparison of the CAN gene signature of the tumor and information in the database. Exemplary healthcare providers include doctors, nurses, and nurse practitioners. Diagnostic laboratories can also provide a recommended therapy based on signatures and other information about the patient.

Following treatment with a primary cancer therapy, the patient can be monitored for an improvement or worsening of the cancer. A tumor tissue sample (such as a biopsy) can be taken at any stage of treatment. In particular, a tumor tissue sample can be taken upon tumor progression, which can be determined by tumor growth or metastasis. A CAN gene signature can be determined, and one or more secondary therapeutic agents can be administered to increase, or restore, the sensitivity of the tumor to the primary therapy.

Treatment predictions may be based on pre-treatment gene signatures. Secondary or subsequent therapeutics can be selected based on the subsequent assessments of the patient and the later signatures of the tumor. The patient will typically be monitored for the effect on tumor progression.

A medical intervention can be selected based on the identity of the CAN gene signature. For example, individuals can be sorted into subpopulations according to their genotype. Genotype-specific drug therapies can then be prescribed. Medical interventions include interventions that are widely practiced, as well as less conventional interventions. Thus, medical interventions include, but are not limited to, surgical procedures, administration of particular drugs or dosages of particular drugs (e.g., small molecules, bioengineered proteins, and gene-based drugs such as antisense oligonucleotides, ribozymes, gene replacements, and DNA- or RNA-based vaccines), including FDA-approved drugs, FDA-approved drugs used for off-label purposes, and experimental agents. Other medical interventions include nutritional therapy, holistic regimens, acupuncture, meditation, electrical or magnetic stimulation, osteopathic remedies, chiropractic treatments, naturopathic treatments, and exercise.

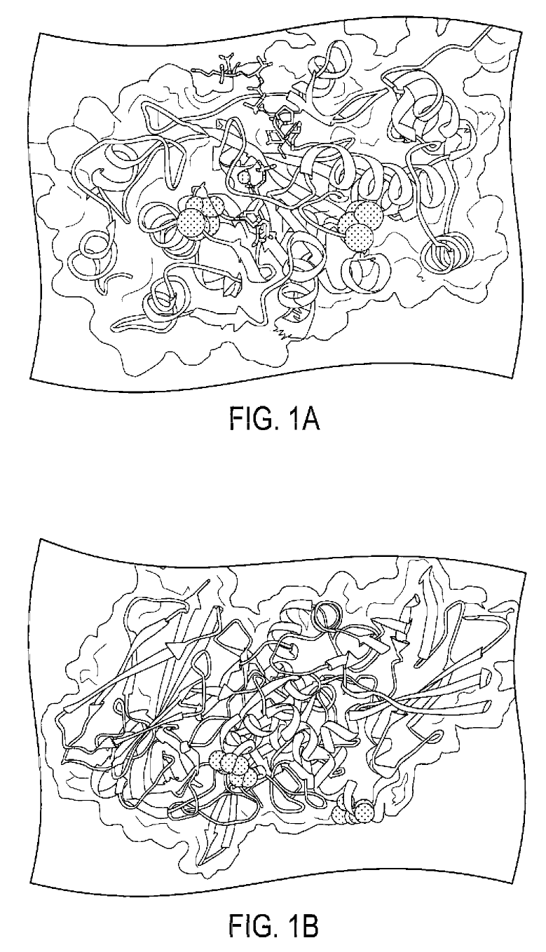

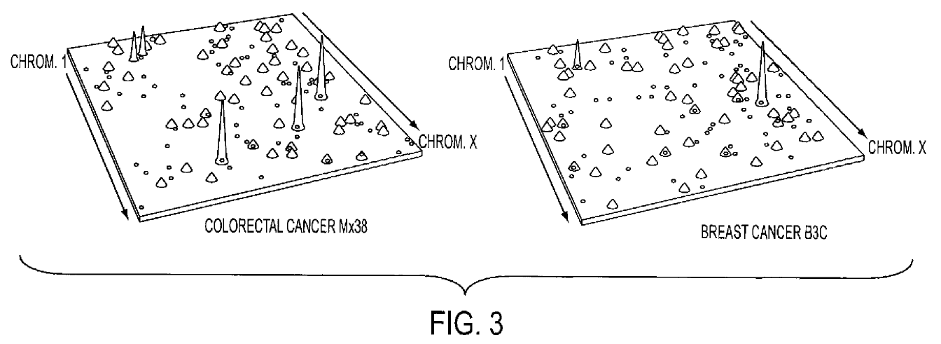

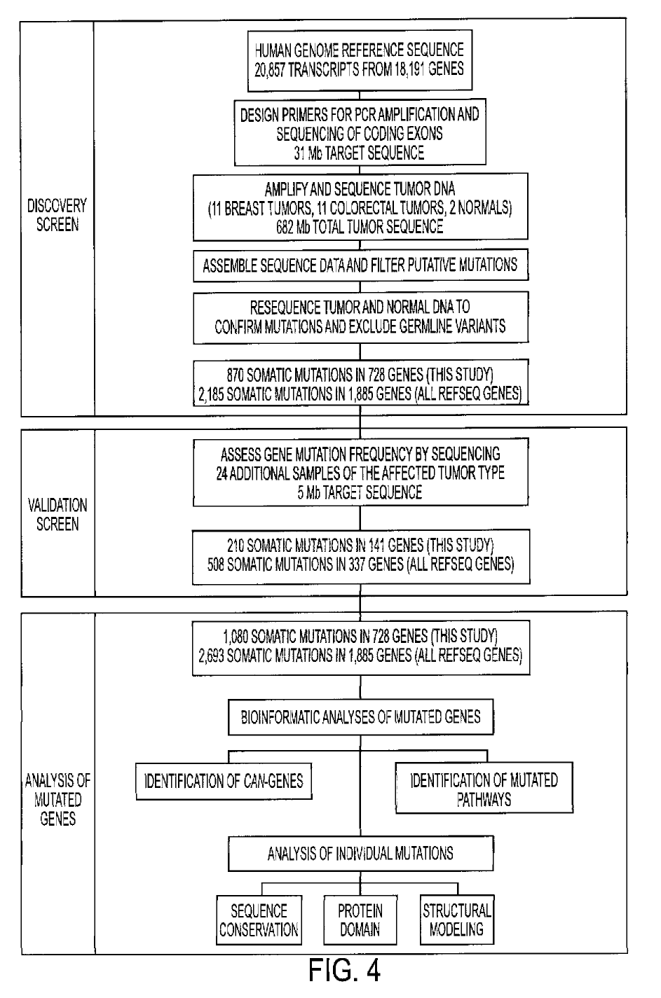

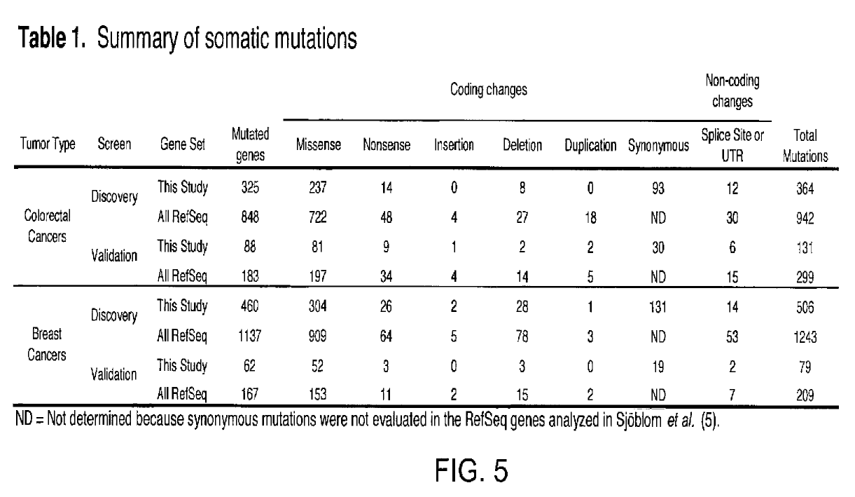

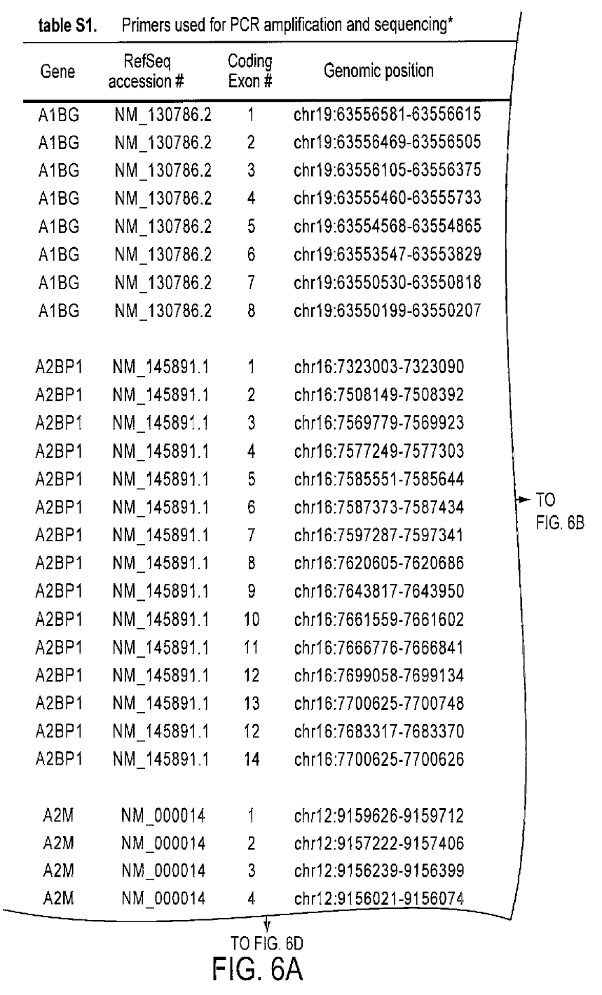

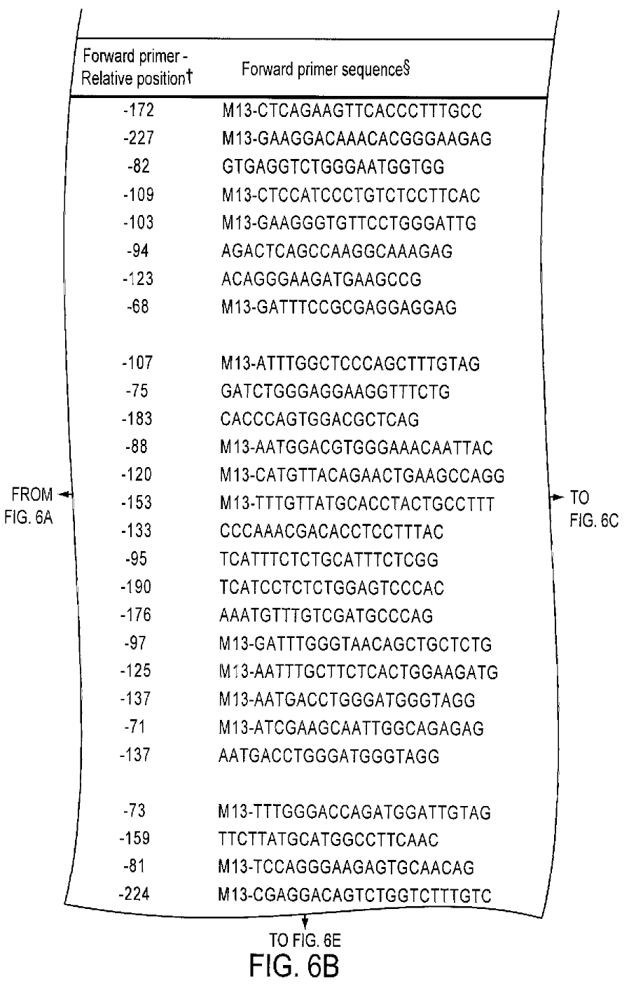

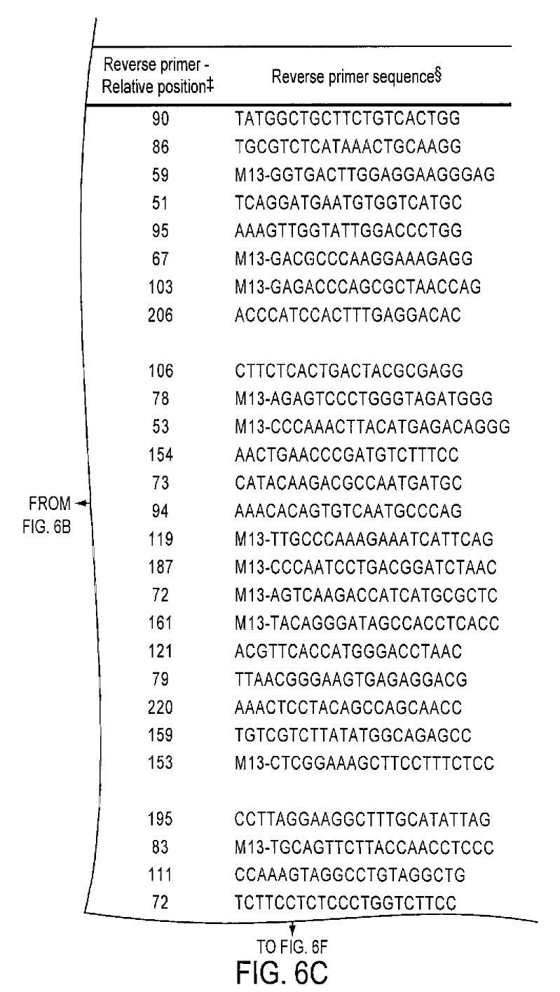

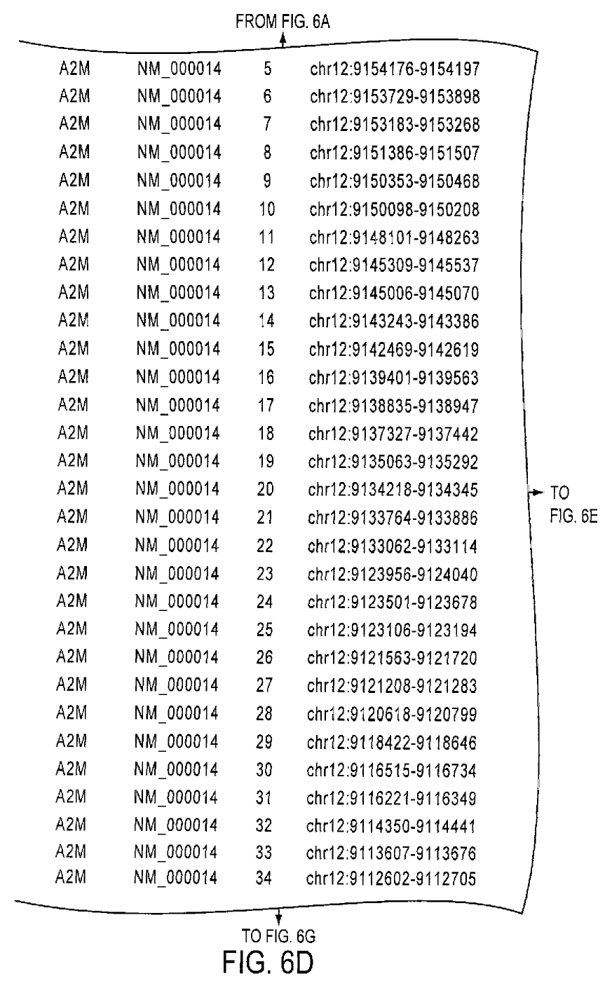

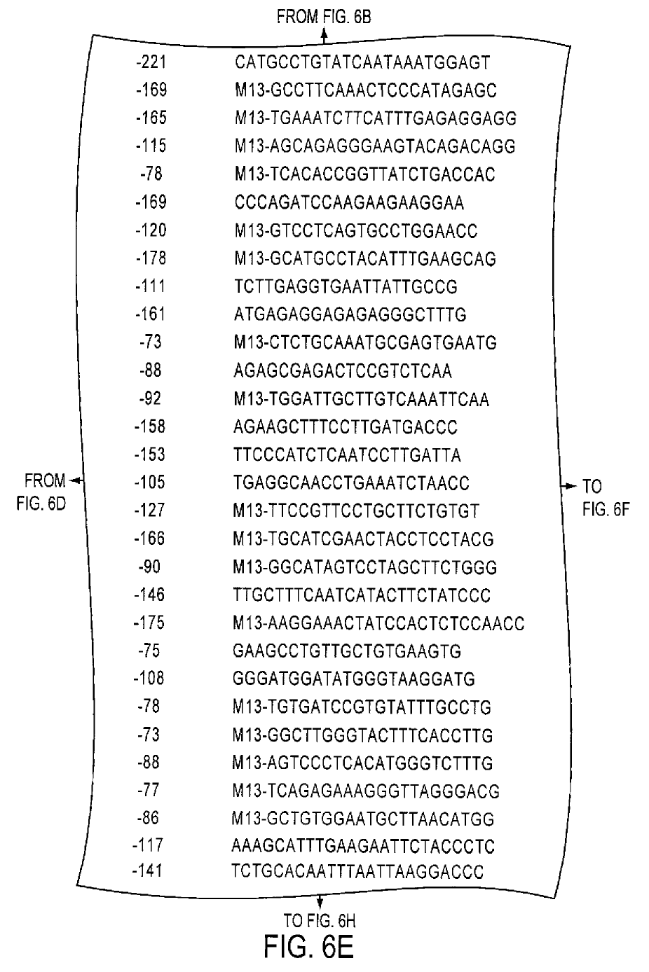

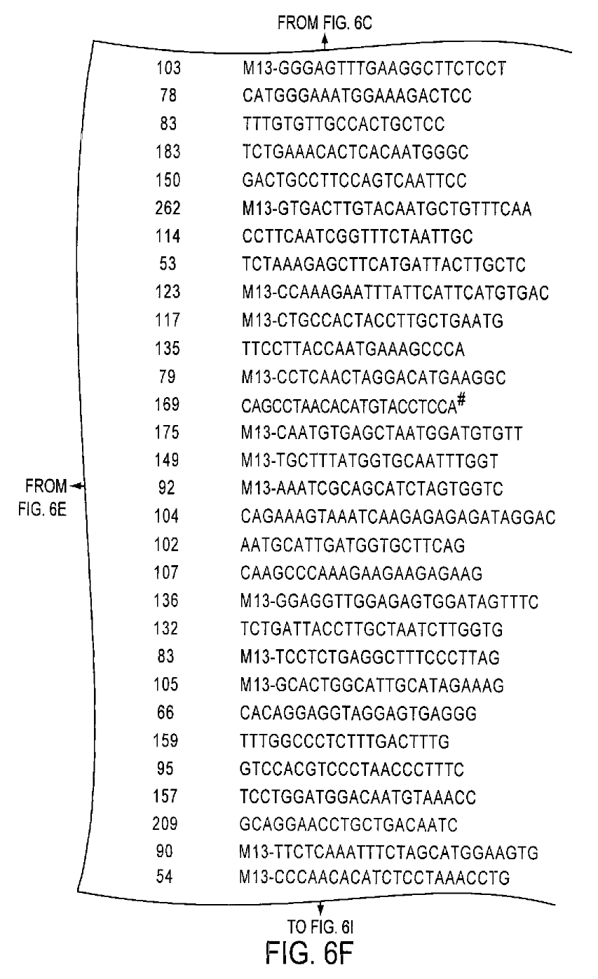

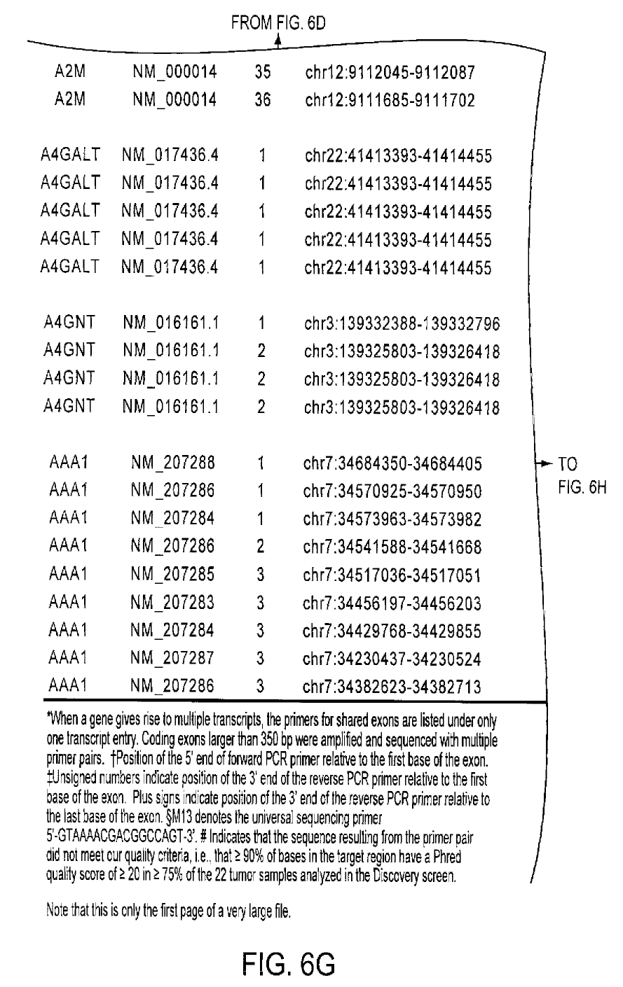

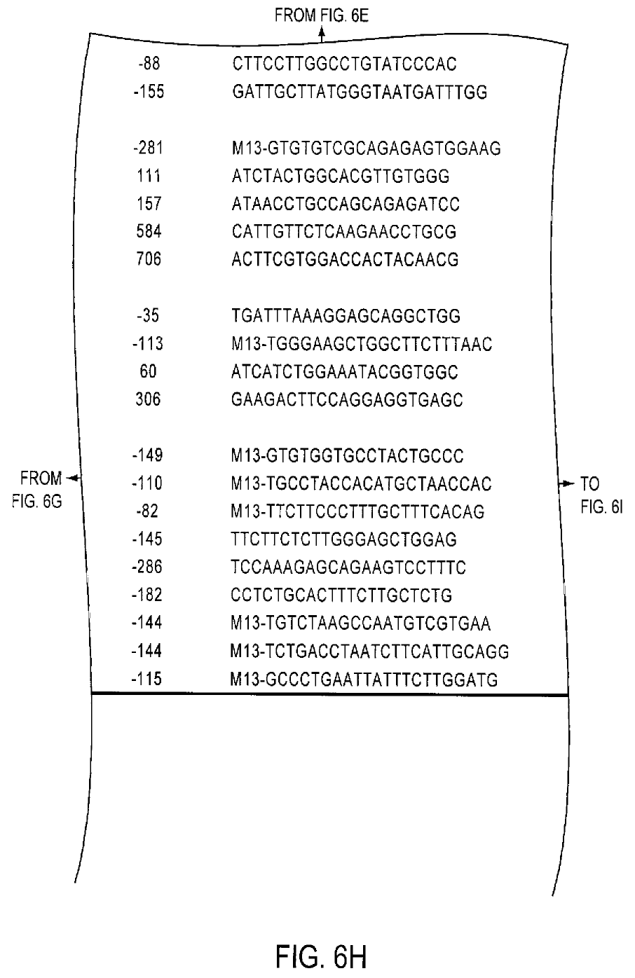

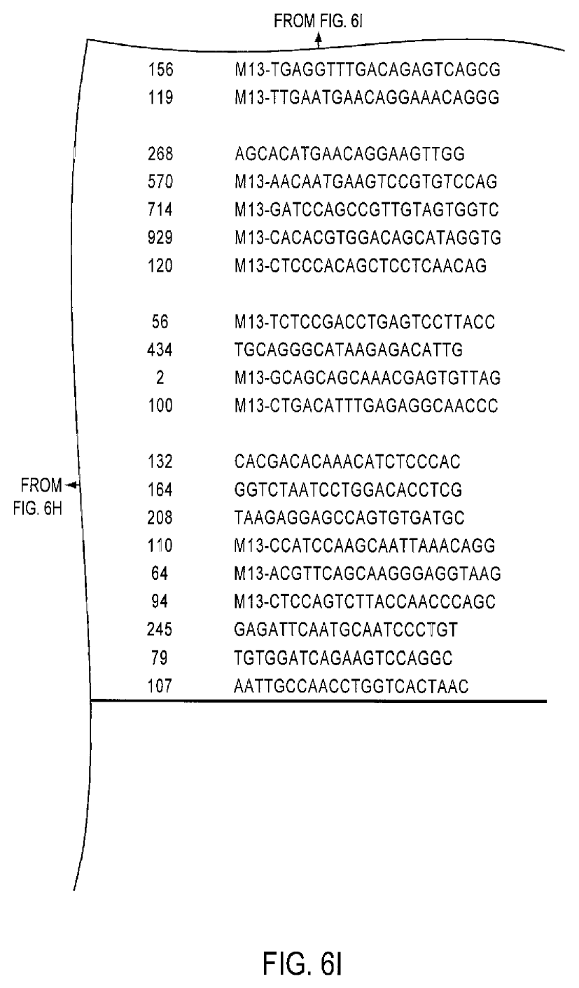

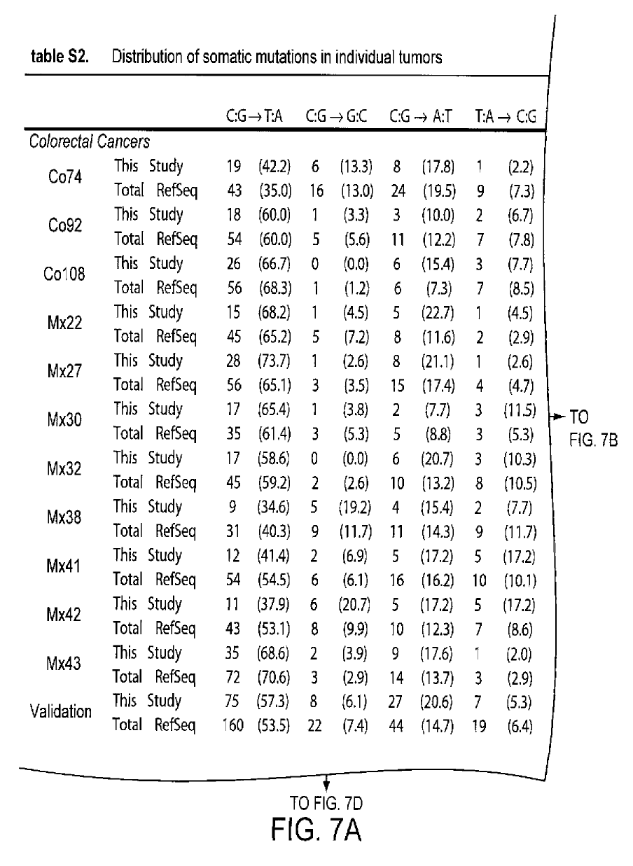

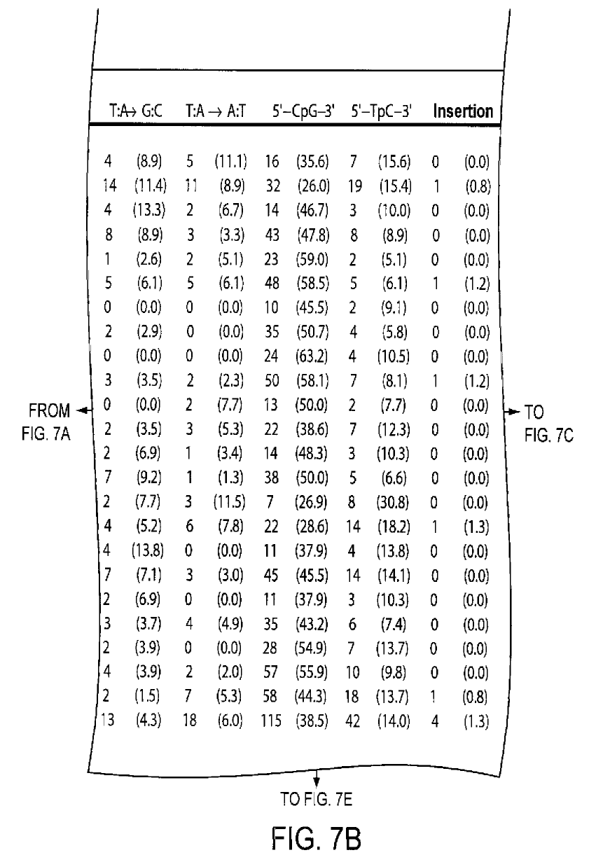

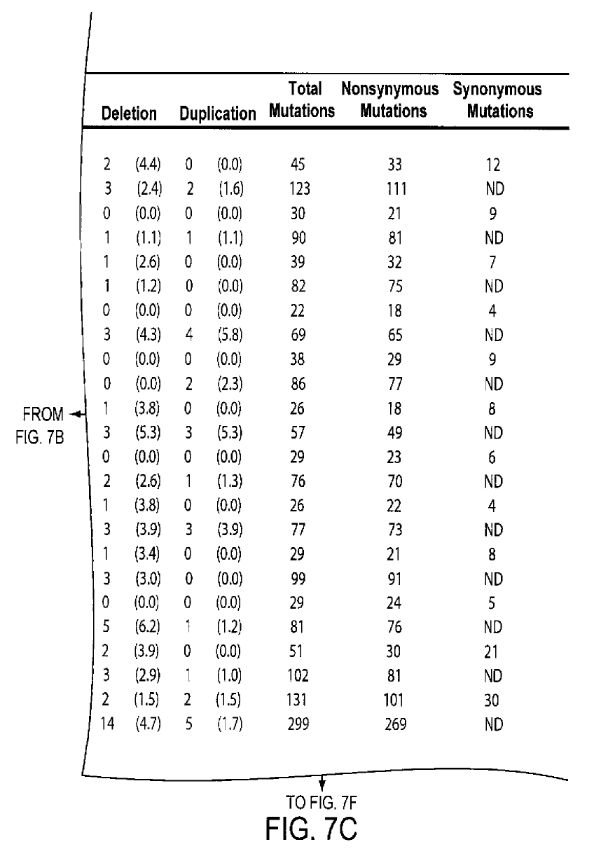

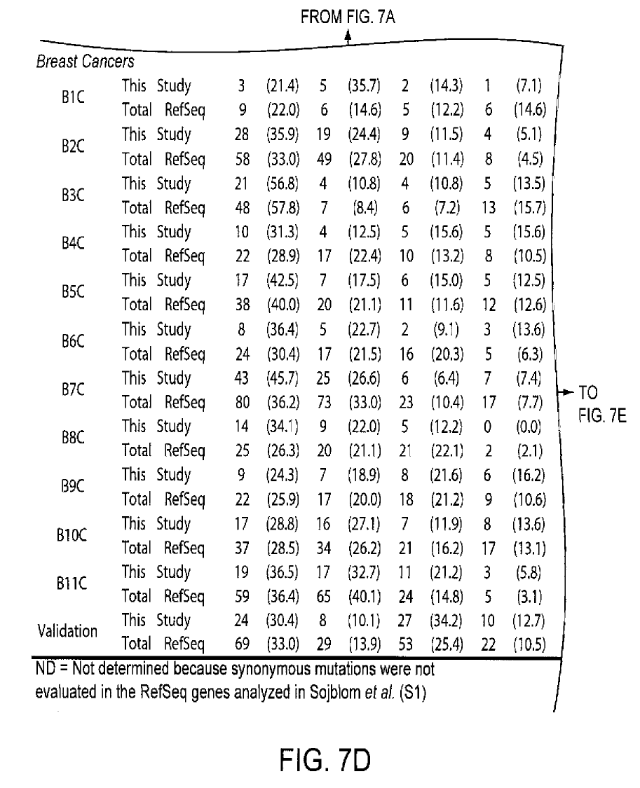

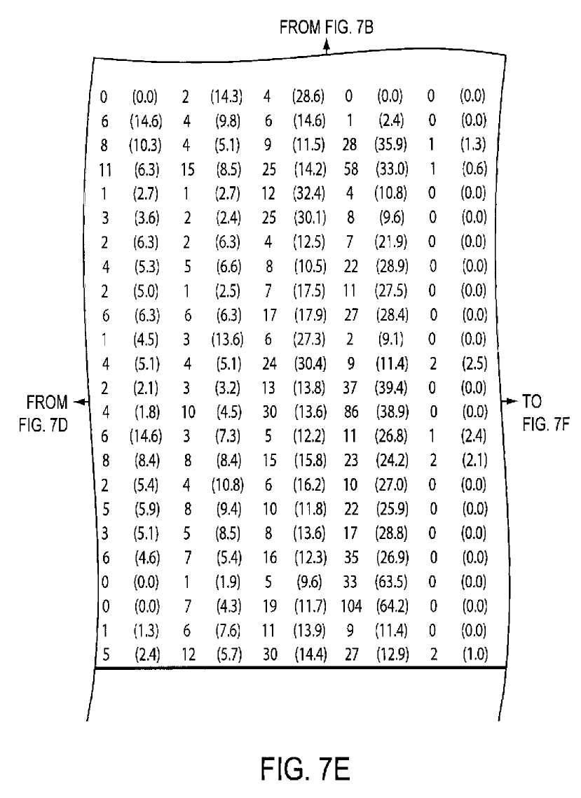

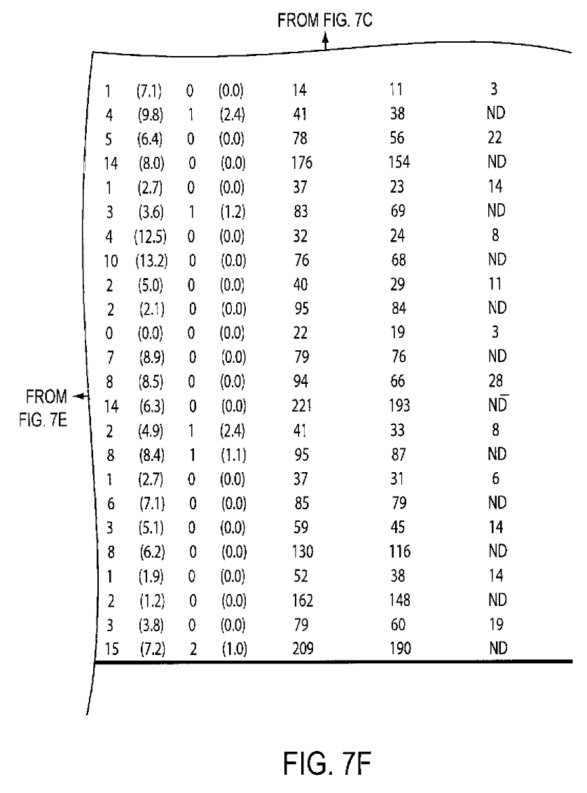

We report the sequences of an additional 5,168 genes in 22 tumors. These new data provide a much more complete picture of the cancer genome, allowing us to formulate landscapes of breast and colorectal tumors (FIG. 3). We predict that the key features of this landscape—a few gene mountains interspersed with many gene hills—will prove to be a general feature of most solid tumors. We also present data on non-coding and synonymous mutations in addition to non-synonymous mutations. As well as providing information useful for estimating the passenger rate, the data in table S2 shows that passenger rates vary considerably from tumor to tumor, undoubtedly determined by their intrinsic mutability and the number of generations and bottlenecks through which they have evolved. We also present more sophisticated methods for identifying and classifying genes with more mutations than predicted by the passenger rate FIGS. 9, 10, (table S4). Additionally, we present a variety of tools based on gene products' sequence and structure, as well as their inclusion in certain pathways, that can help identify mutated genes that are most deserving of further attention (FIGS. 1, 2, 8, 9, 10, 12, 13 (tables S3, S4, S6)). These tools can be used to prioritize the research that follows cancer genome sequencing efforts.

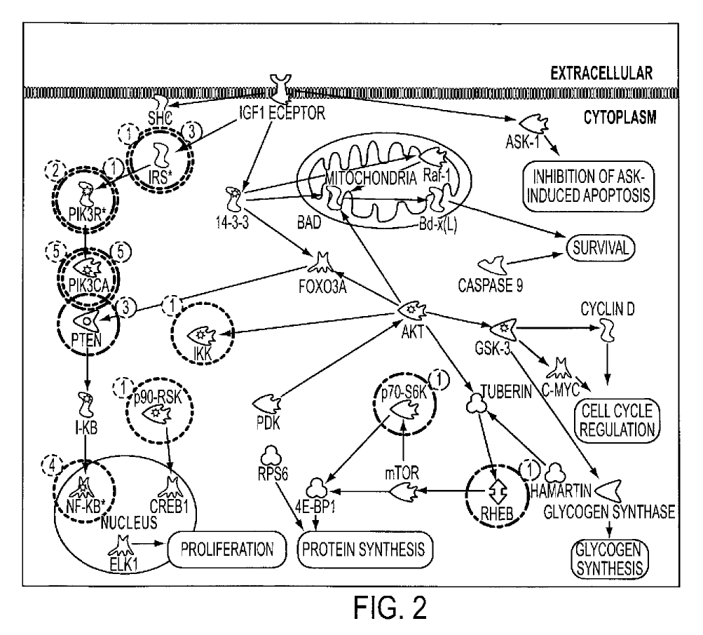

In terms of such research, it is important to note that sequence data can inform other, independent approaches to the study of cancer genes. For example, chromodomain helicase DNA binding domain 5 (CHD5) was recently proposed to be a tumor suppressor based on its functional properties and copy number alterations (22). We identified somatic mutations in this gene in breast tumors; the combined data strongly support a role for this gene in tumorigenesis. Similarly, the NF-κB pathway member IKBKE was recently suggested to be a breast cancer oncogene based on functional and expression studies (23). We found somatic mutations in several additional components of this signaling pathway (FIG. 2), reinforcing its importance in breast cancers. The transglutaminase (TGM) enzymes have recently been implicated in invasion and metastasis (24), and we identified multiple somatic mutations in TGM3 in colorectal cancers (FIG. 1). Additionally, a high-throughput retroviral insertional mutagenesis screen in MMTV-induced mammary tumors in mice identified 33 common insertion sites as potential oncogenes (25); we found seven of these 33 genes to be mutated in breast cancers. Given the entirely independent nature of these screens (insertional mutagenesis in mouse vs. mutational analysis of human genes), these results are remarkable.

Historically, the focus of cancer research has been on the gene mountains, in part because they were the only alterations identifiable with available technologies. The ability to analyze the sequence of virtually all protein-encoding genes in cancers has shown that the vast majority of mutations in cancers, including those that are most likely to be drivers, do not occur in such mountains and emphasize the heterogeneity and complexity of human neoplasia. This new view of cancer is consistent with the idea that a large number of mutations, each associated with a small fitness advantage, drive tumor progression (26). But is it possible to make sense out of this complexity? When all the mutations that occur in different tumors are summed, the number of potential driver genes is large. But this is likely to actually reflect changes in a much more limited number of pathways, numbering no more than 20 (1). This interpretation is consistent with virtually all screens in model organisms, which have generally shown that the same phenotype can arise from alterations in any of several genes. Other recent studies lend support to this interpretation. For example, sequencing studies of the kinome in large numbers of tumors have shown that specific kinases are sometimes mutated in a small fraction of tumors of a given type (4, 10, 27-29). We cannot be certain that the bulk of the low frequency mutations observed in our study are not passengers. However, in the kinome studies, the position of mutations within the activation loop and the demonstrated effects of the target residues on kinase function unambiguously implicate many of these rare mutations as drivers. Similarly, recent analyses of myelomas suggest that there are multiple genes, each mutated in a small proportion of tumors, that can alter the same signal transduction pathway (30, 31). And some of the low frequency mutations observed in our study, such as activating mutations in the guanine nucleotide binding protein GNAS and a homozygous nonsense mutation in BRCA1-associated protein (BAP1), are likely to be functional (table S3). These examples, in addition to those in table S6, bolster the argument that infrequent mutations can be drivers and that they function through pathways that are already known.

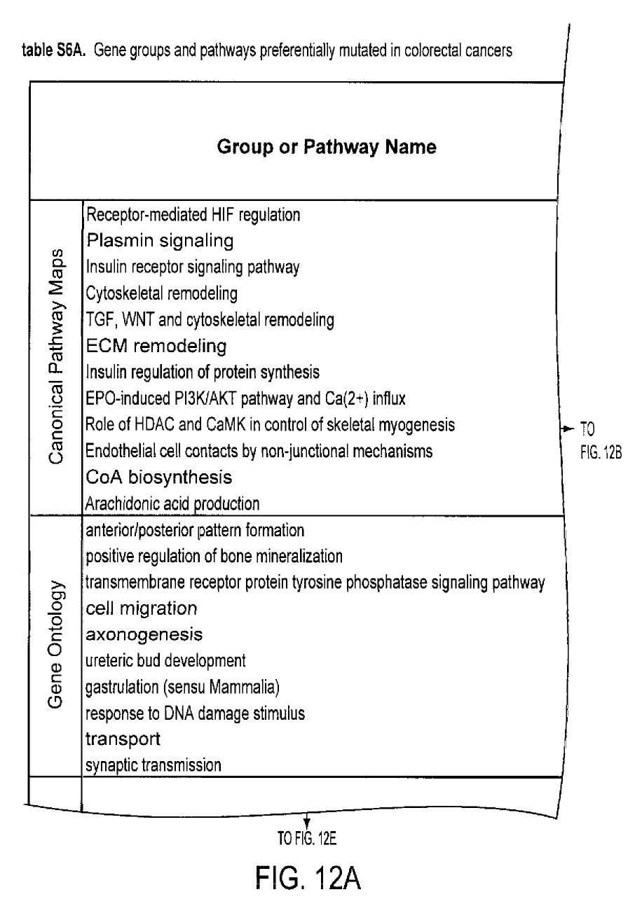

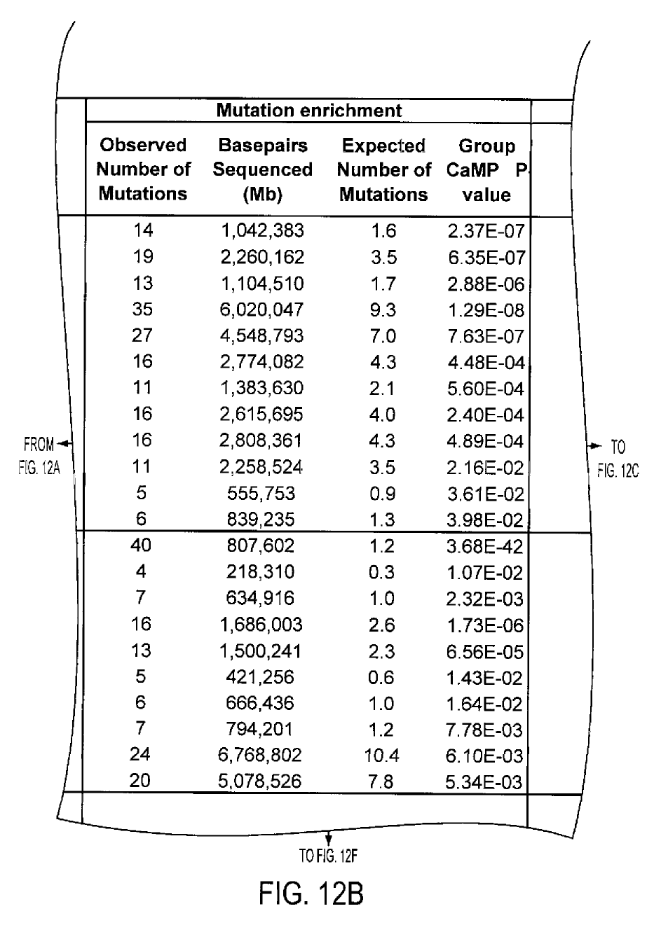

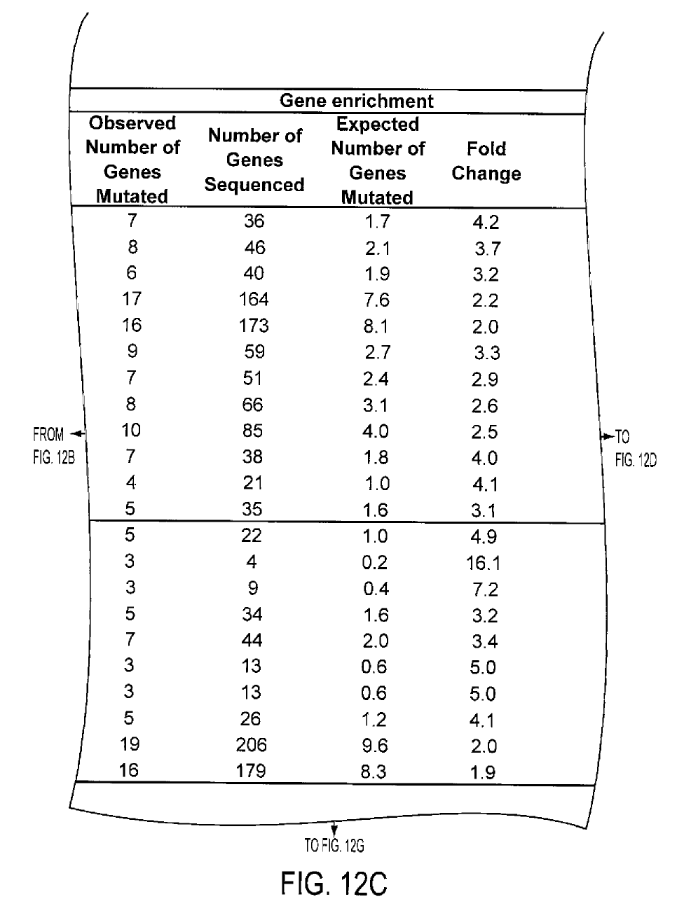

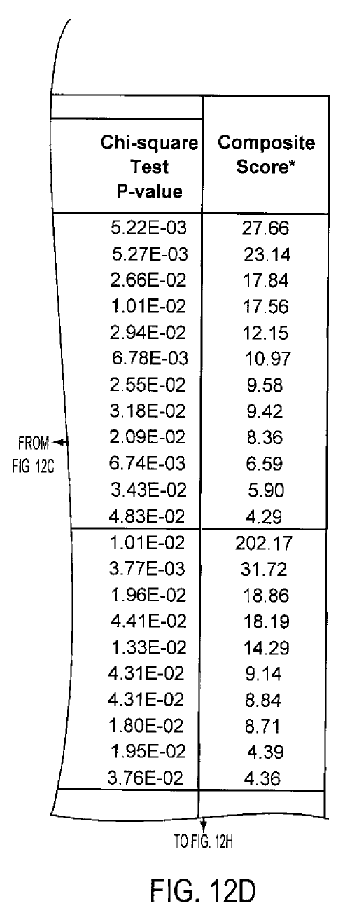

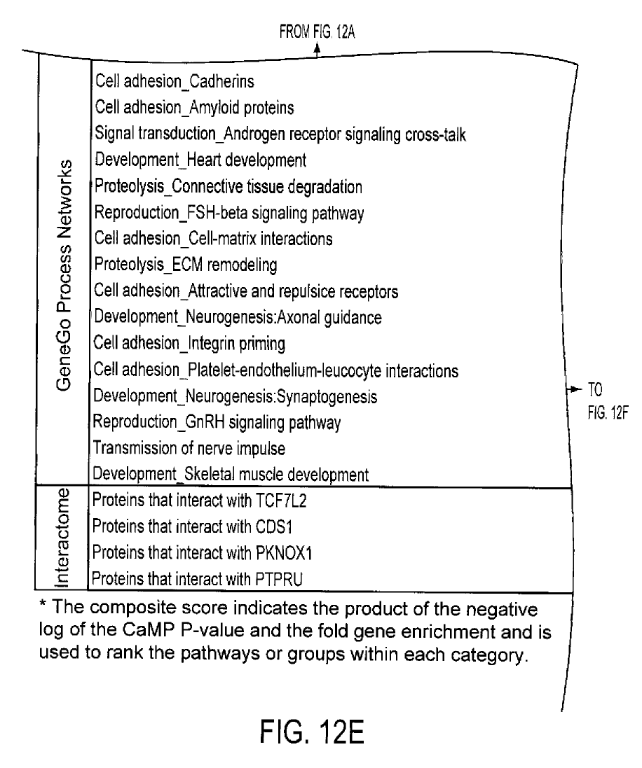

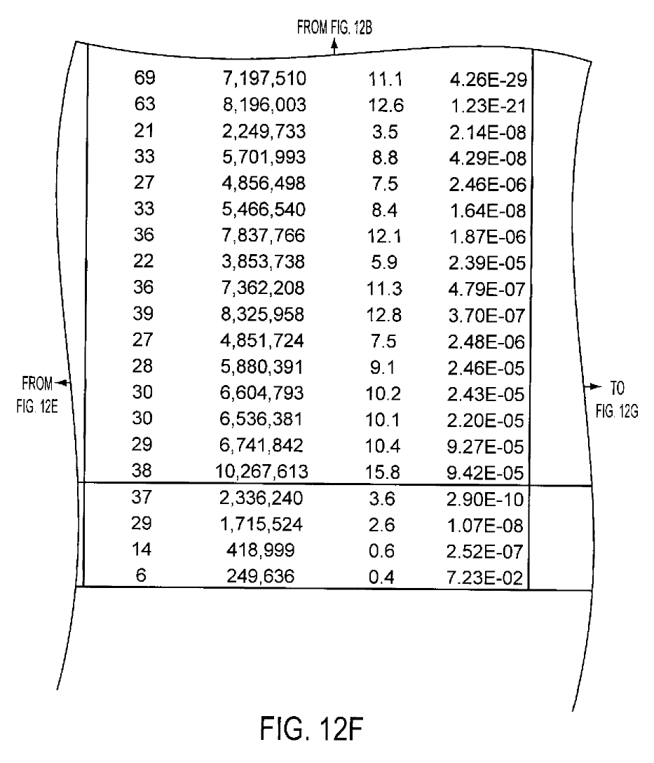

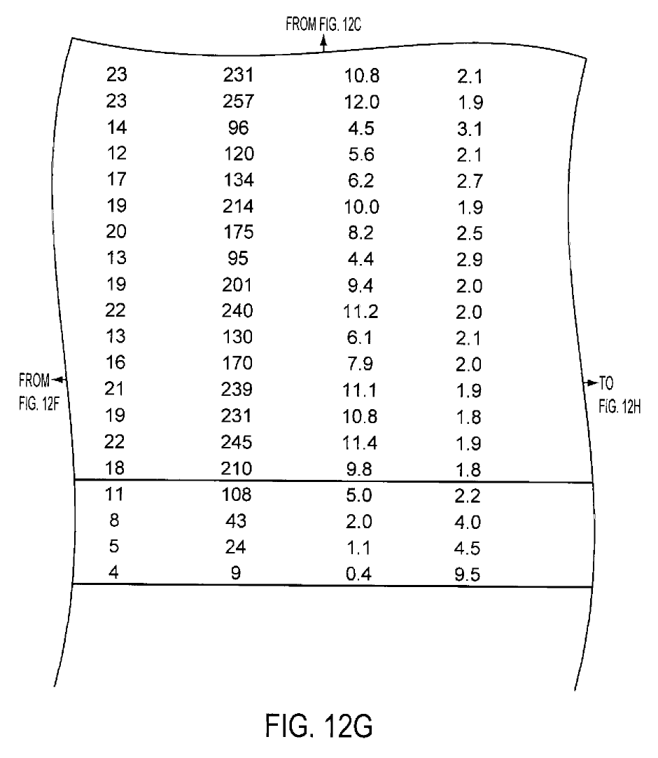

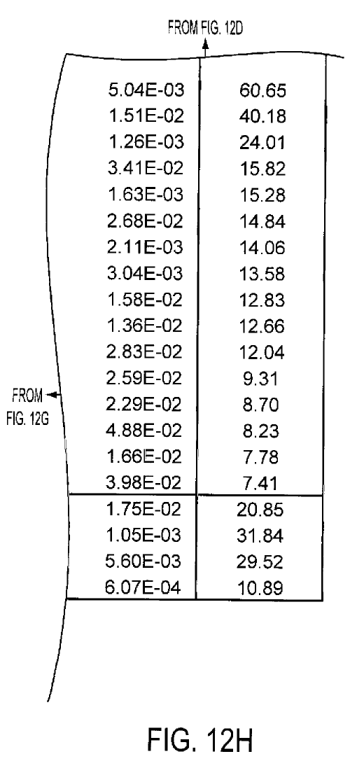

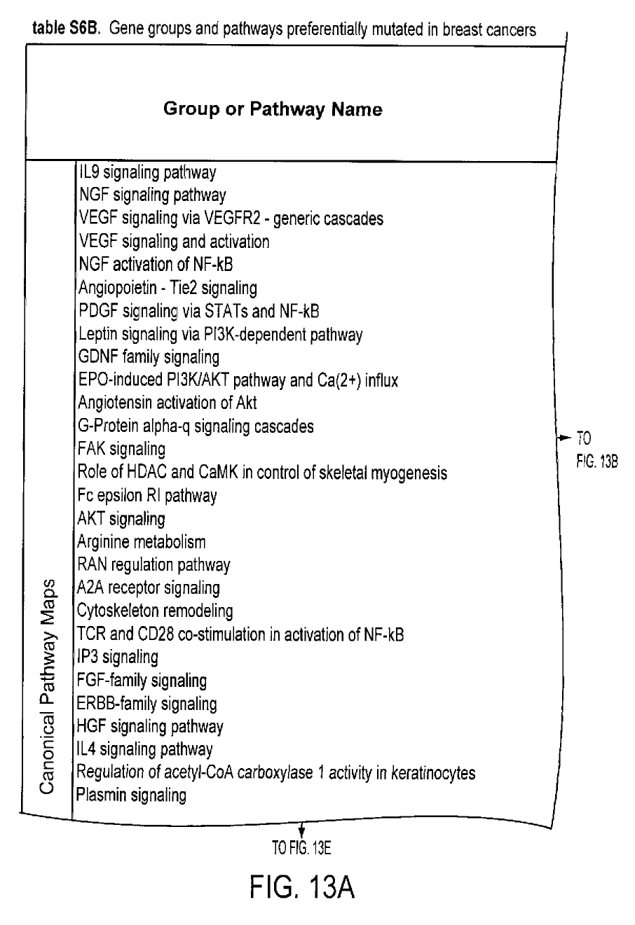

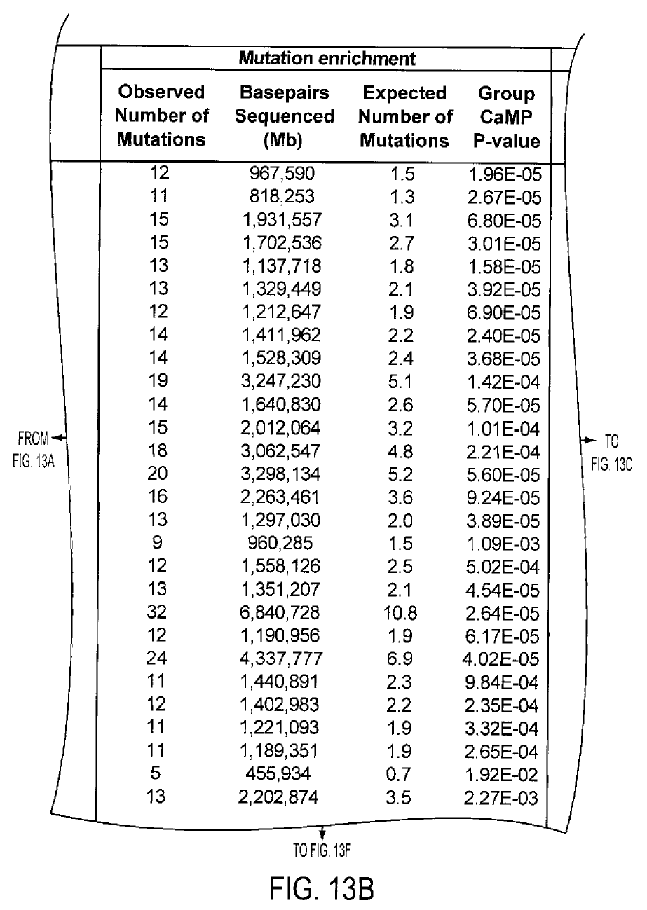

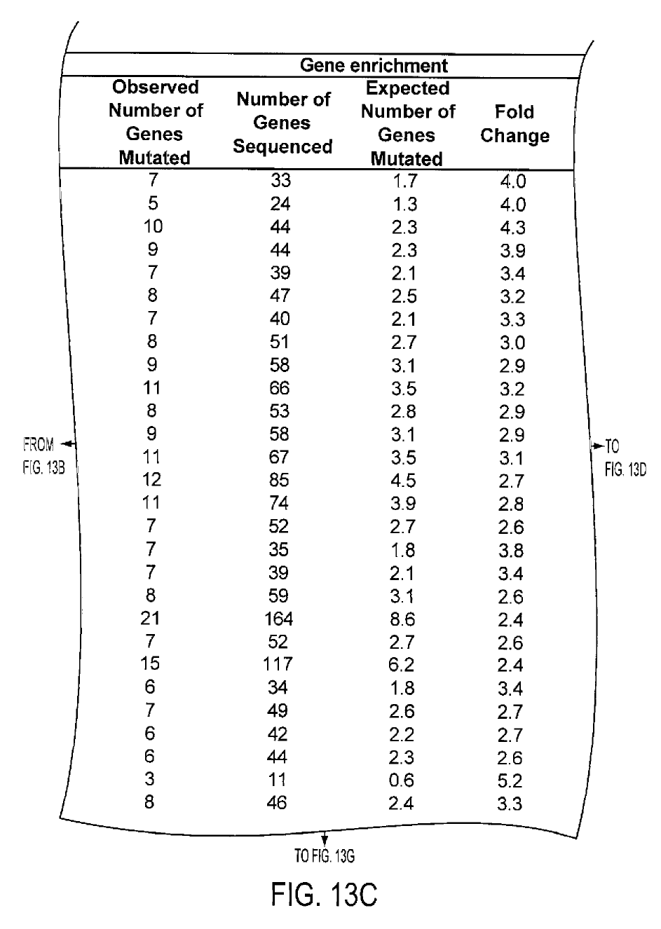

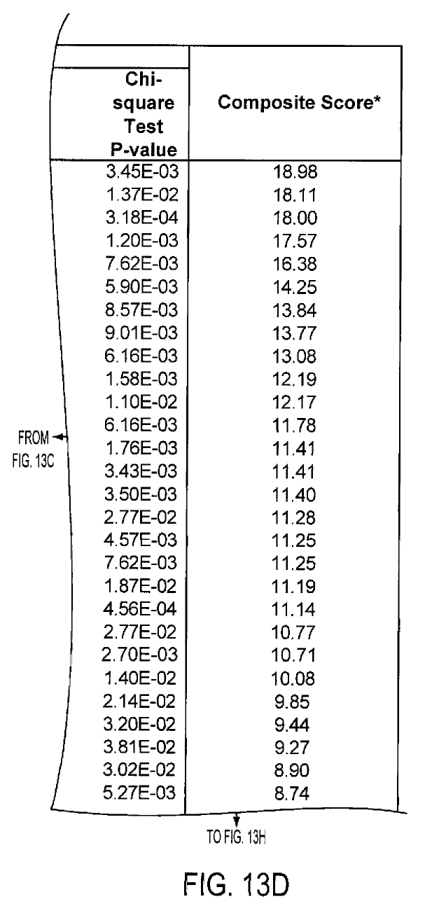

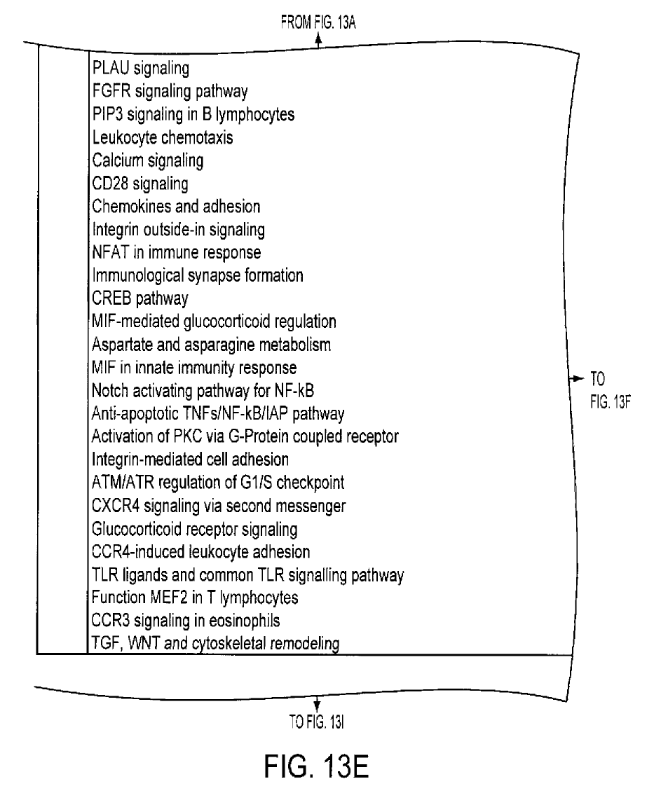

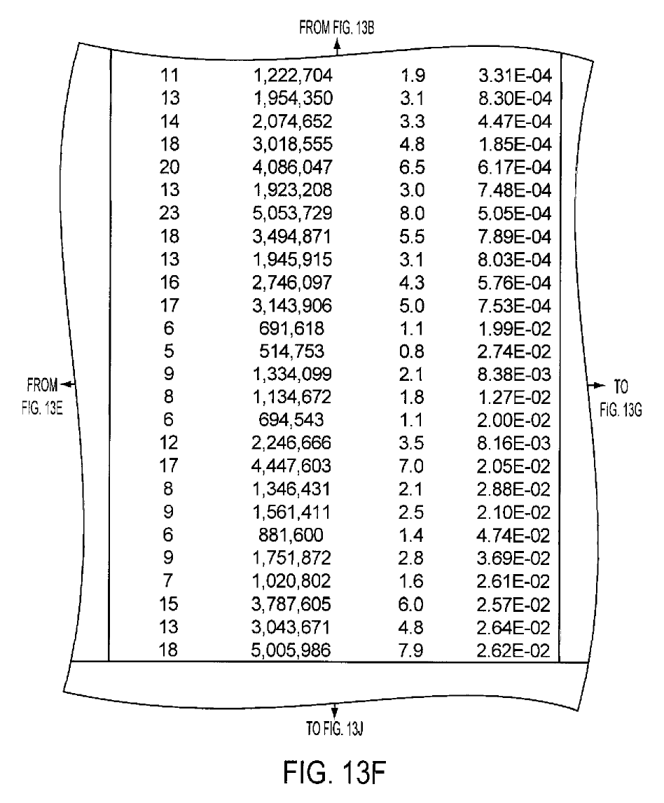

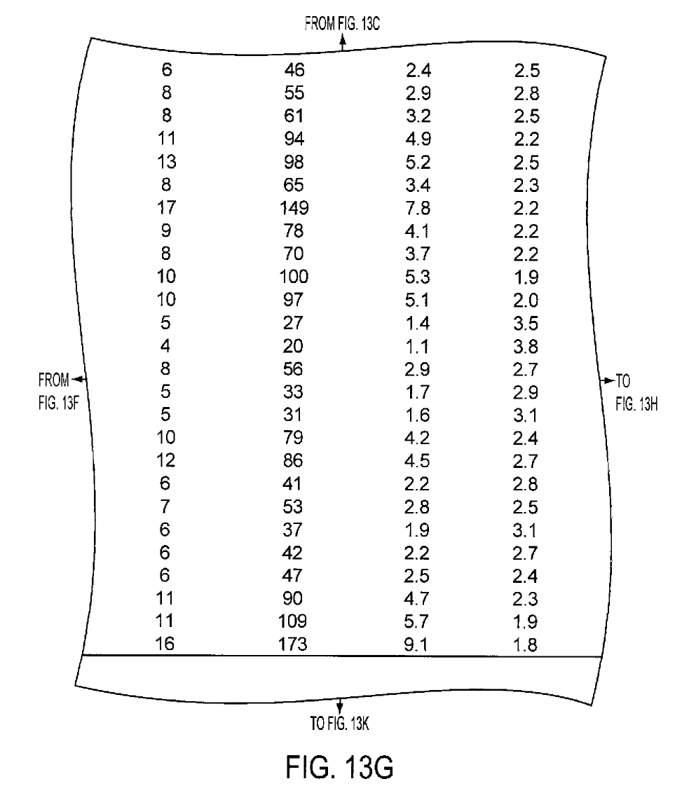

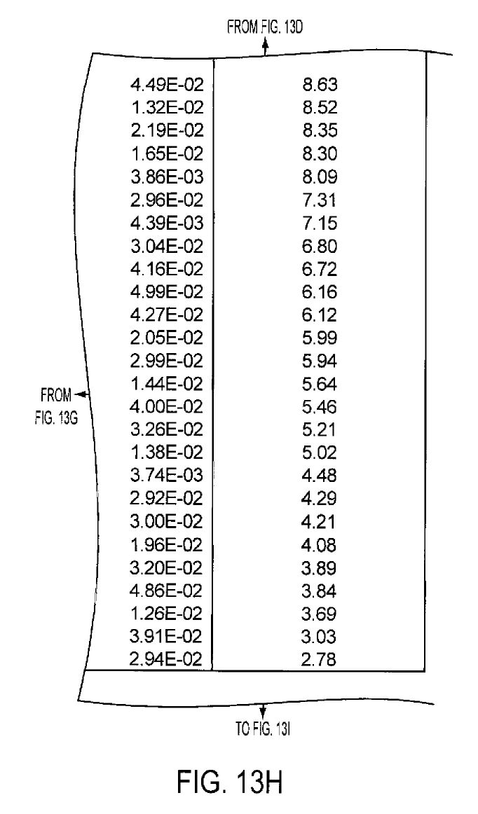

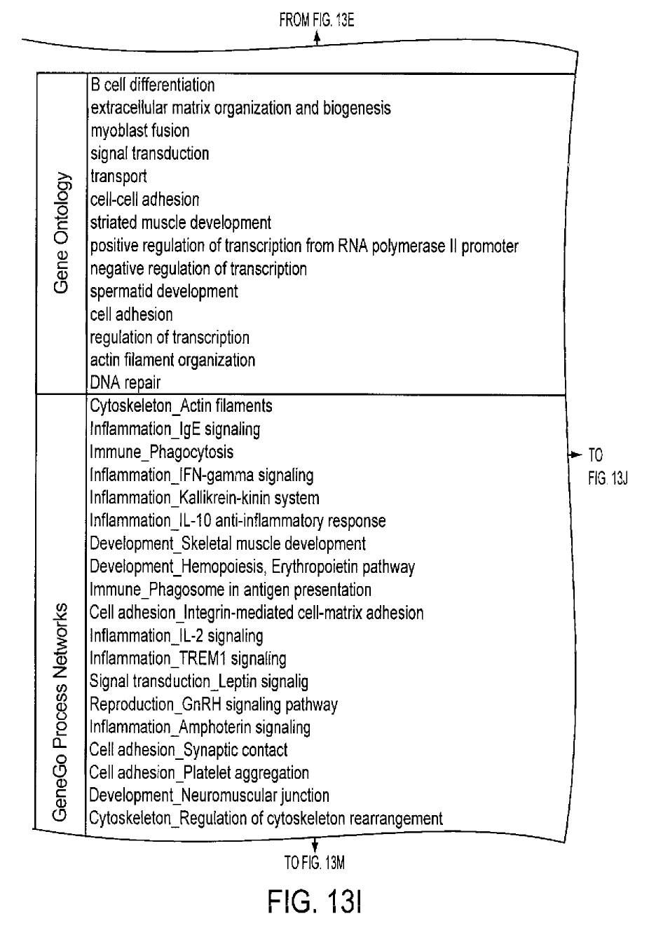

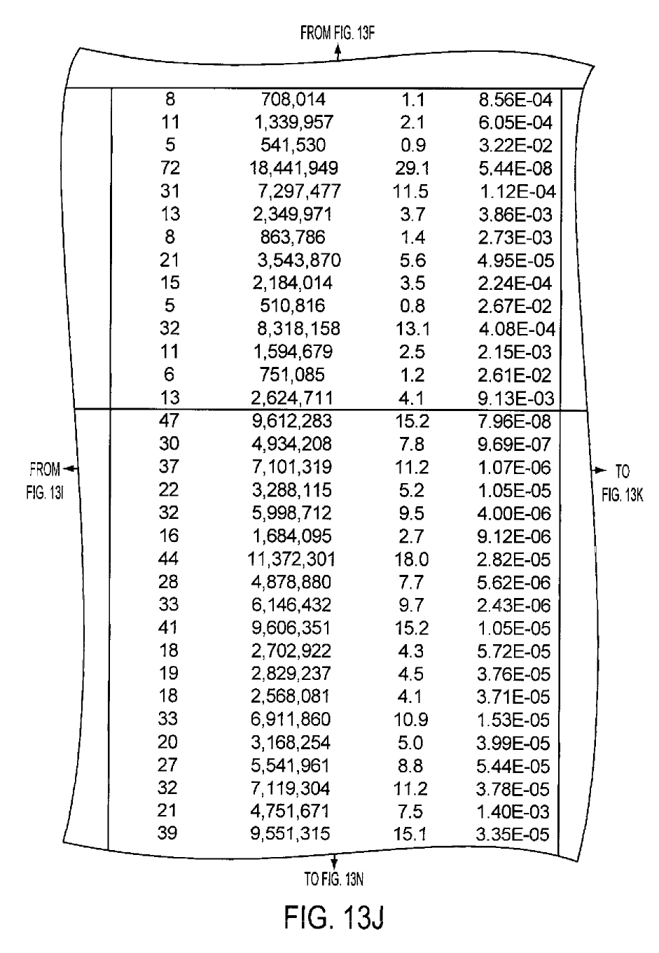

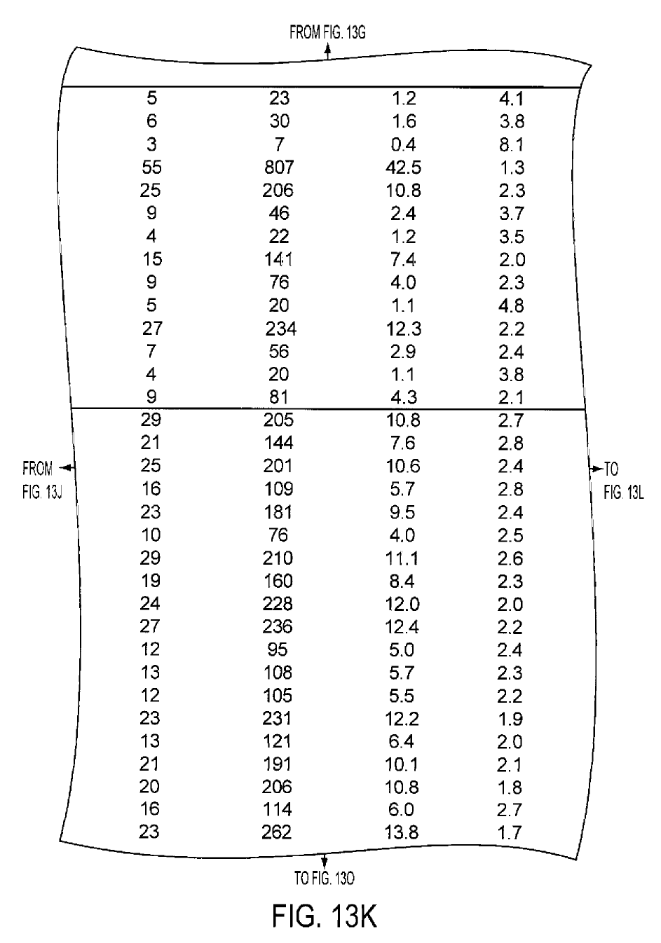

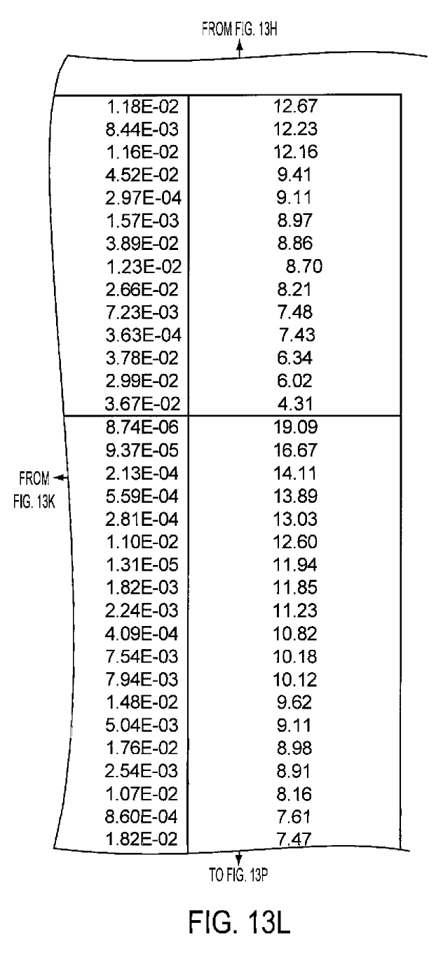

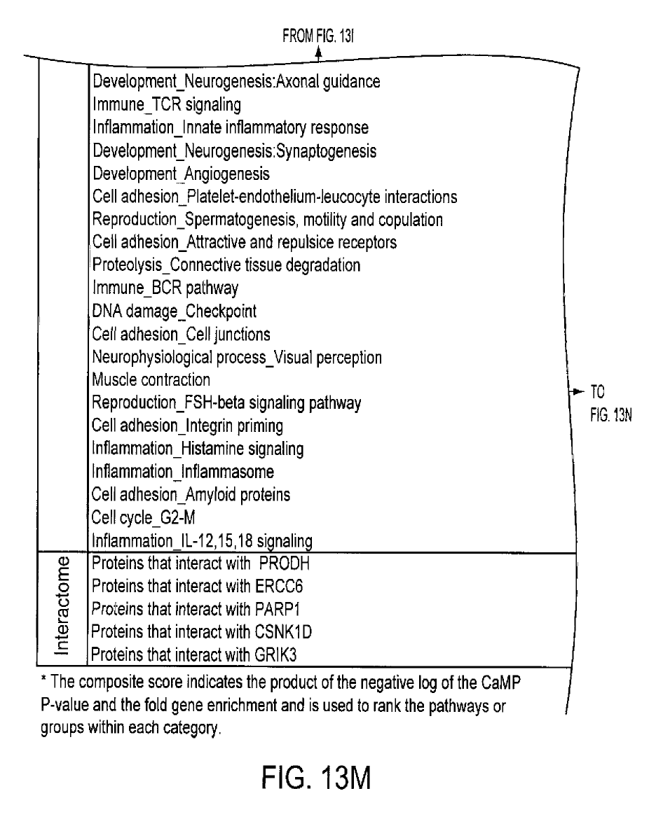

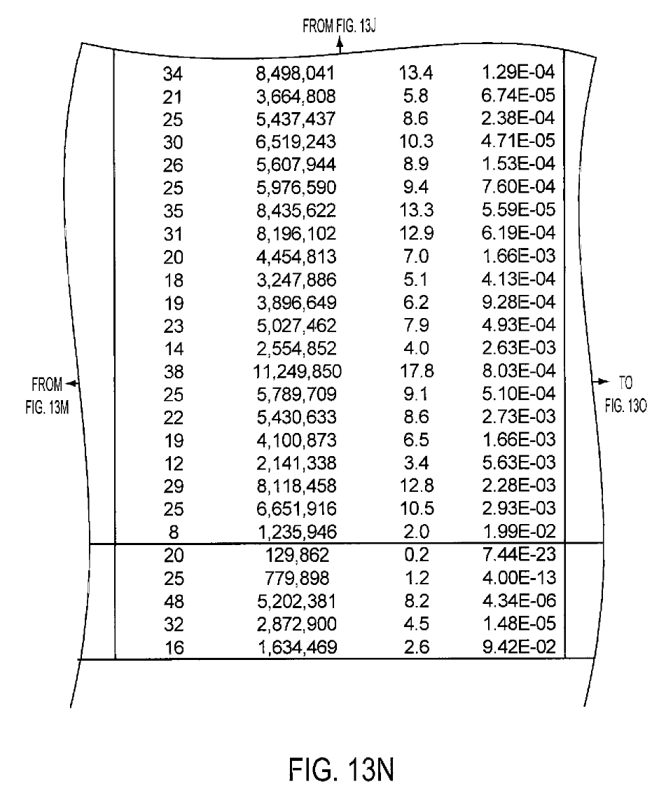

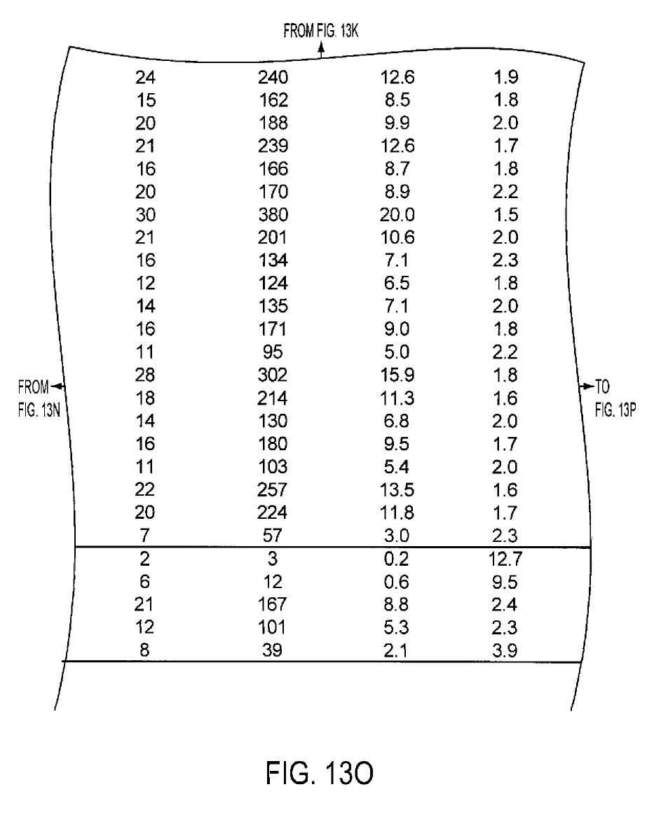

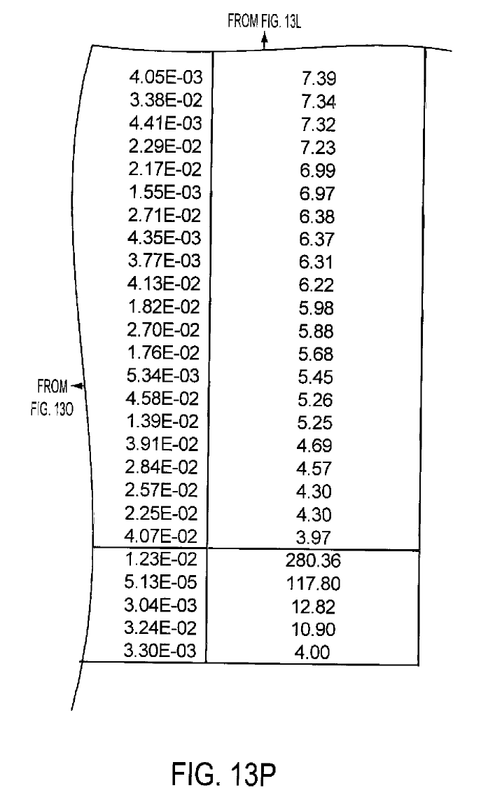

Regardless of whether this pathway-centric interpretation is correct, it is clear that the “easy” part of future cancer genome research will be the identification of genetic alterations. The vast majority of subtle mutations in individual patients' tumors can now be identified with existing technology (FIG. 3), making personal cancer genomics a reality. Though understanding the precise role of these genetic alterations in tumorigenesis will be more challenging, opportunities for exploiting such personal genomic data on cancers are already apparent. For example, many of the genes altered in breast cancers appear to affect the NF-κB pathway (FIGS. 12, 13; table S6), suggesting that drugs targeting this pathway could be efficacious in breast cancers with such mutations (30, 31). Furthermore, our data indicate that individual breast and colorectal cancers each contain an average of ˜90 amino acid-altering mutations that are absent in all normal cells, providing a wealth of opportunities for personalized immunotherapy. Finally, any mutation identified in an individual cancer, whether driver or passenger, can be used as an exquisitely specific biomarker to guide patient management (32).

The above disclosure generally describes the present invention. All references disclosed herein are expressly incorporated by reference. The disclosure of international application PCT/US07/017,866 filed Aug. 13, 2007, is expressly incorporated by reference. A more complete understanding can be obtained by reference to the following specific examples which are provided herein for purposes of illustration only, and are not intended to limit the scope of the invention.

### EXAMPLES

### Example 1

### Sequencing Strategy

The first step in our approach was the design of primers that would permit polymerase chain reaction (PCR)-based amplification and analysis of coding exons in the RefSeq database. Of the 20,857 transcripts in the RefSeq database (representing 18,191 distinct genes), 14,661 transcripts were included in the CCDS set. These CCDS genes were in general not evaluated again; the only exceptions were a small subset in which particular regions of interest had been difficult to amplify and for these, new PCR primers were designed. For the remaining 6,196 Refseq transcripts, 125,624 primers were designed and used to amplify the coding exons. The entire list of primers used to amplify the exons of the RefSeq genes (including the CCDS genes) is provided in table S1.

The primers were used to PCR-amplify and sequence the DNA from 11 breast and 11 colorectal cancers as well as DNA from matched normal tissues of two patients. The samples used for this analysis were the same as those used in the previous study of CCDS genes (5). The sequence data from this Discovery Screen were assembled and evaluated using stringent quality criteria (7), resulting in successful analysis of 93% of targeted amplicons. We used bioinformatic and experimental strategies to distinguish germline variants and artifacts of PCR or sequencing from true somatic mutations (FIG. S1). Genetic alterations found in the two normal samples and those present in SNP databases were removed and sequence traces of the remaining potential alterations were visually inspected to remove false positive calls in the automated analysis. After these steps, the amplicons of the remaining alterations were re-amplified from the tumor DNA (to ensure reproducibility) and from DNA of matched normal tissue (to remove unannotated germline variants). Finally, the putative somatic mutations were examined in silico to ensure that the alterations did not occur as a result of mistargeted amplification of related regions of the genome (7).

To further evaluate the genes with somatic mutations in the Discovery Screen, we determined their sequence in a Validation Screen of 24 additional samples of the same tumor type in which the mutation was originally identified. Similar methods to those noted above were used to exclude germline variants, PCR and sequencing artifacts, and alterations due to mistargeted amplification of related genomic regions. Amplicons with putative somatic mutations were re-amplified in DNA from the tumor and from matched normal tissues to determine whether the alterations were truly somatic.

### Example 2

### Somatic Mutations

Combining the data from the current analysis with those previously obtained in CCDS genes, we found that 1718 genes (9.4% of the 18,191 genes analyzed) had at least one non-silent mutation in either a breast or colorectal cancer (Table 1 and table S3). The great majority of alterations were single base substitutions (92.7%), with 81.9% resulting in missense changes, 6.5% resulting in stop codons, and 4.3% resulting in alterations of splice sites or untranslated regions immediately adjacent to the start and stop codons (Table 1). The remaining somatic mutations were insertions, deletions, or duplications (7.3%). The mutation spectrum of colorectal cancers differed from that of breast cancers, and these spectra were similar to those observed in the previous CCDS study and in other analyses (4, 5). In the current study we analyzed the nature of the non-synonymous mutations in more detail and found a very large excess of C to T transitions at 5′-CpG-3′ in colorectal cancers, representing 19-fold more than expected from the representation of 5′-CpG-3′ sites in the coding regions of the genome. Similarly, there was a marked excess of G to C transversions at 5′-GpA-3′ sites in breast cancers, representing 4.5 fold more than expected (7).

### Example 3

### Passenger Mutation Rates

The somatic mutations found in cancers are either “drivers” or “passengers” (4). Driver mutations are causally involved in the neoplastic process and are positively selected for during tumorigenesis. Passenger mutations provide no positive or negative selective advantage to the tumor but are retained by chance during repeated rounds of cell division and clonal expansion.

We used two independent methods to estimate the passenger mutation rates in the analyzed cancers. First, we evaluated 23.8 Mb of chromosome 8 in eleven colorectal cancer samples similar to those used in the Discovery Screen. This was performed with high density oligonucleotide microarrays containing every possible single base pair substitution. The tumors used for this analysis each had only one allele of chromosome 8 (i.e. they showed loss of heterozygosity), rendering the detection of sequence alterations sensitive and reliable. A total of 151 somatic mutations were identified in 262 Mb of tumor DNA, and all but one of these were located in non-coding regions. Thus, there were a total of 0.6 non-coding mutations per Mb analyzed (95% CI: 0.52 to 0.64 mutations/Mb). Because only one copy of chromosome 8 was analyzed in these studies, the non-coding mutation rate per diploid genome was inferred to be 1.2 mutations/Mb. We then performed detailed LOH analyses of the 11 tumors used in the Discovery Screen using 317,503 polymorphisms. An average of 16% of polymorphic alleles showed LOH. It is known from studies of human genetic variation that the frequency of nonsynonymous (amino acid changing) mutations is approximately half that of mutations in non-coding regions (8, 9). After correcting for loss of heterozygosity and the difference in mutation rates between non-coding and nonsynonymous mutations, these analyses result in an estimated passenger mutation rate of 0.55 nonsynonymous mutations per Mb tumor DNA in colorectal cancers (7). We consider this a minimum estimate because the ratio of mutations in non-coding regions to non-synonymous mutations in coding regions is likely to be higher in the germline than in tumors due to greater negative selection for mutations in coding regions in the germline. Although we have not directly measured mutation rates in non-coding sequences in breast cancers, Stephens et al. have estimated that the rate of non-synonymous mutations in breast cancers is 0.33 per Mb and we used this as our minimum estimate for this tumor type (10).

Estimates of the passenger mutation rates were also obtained through the quantification of synonymous (silent) missense mutations in the current study. As the majority of synonymous changes are expected to be biologically inert and thereby not selected for or against during tumorigenesis, such changes can be used as a tool to estimate passenger mutation rates (11). The analysis of synonymous mutations provided two estimates of the non-synonymous mutation rate (7). One estimate was based on the ratio of non-synonymous to synonymous mutations observed in the human germline (8, 9). The second estimate was derived by calculating the expected ratio of non-synonymous to synonymous changes after accounting for codon usage of RefSeq genes and the different mutation spectra observed in colorectal and breast cancers. We considered this estimate to be a maximum because it did not take into account the fact that nonsynonymous mutations that retard cell growth will be selected against during tumorigenesis.

### Example 4

### Evaluating Mutated Genes

The mutational data obtained can be used to identify candidate cancer genes (CAN-genes) that are most likely to be drivers and are therefore most worthy of further investigation. In the current study, we considered a gene to be a CAN-gene if it harbored at least one nonsynonymous mutation in both the Discovery and Validation Screens and if the total number of mutations per nucleotide sequenced exceeded a minimum threshold (7). Using these criteria, we identified a total of 280 CAN-genes, equally distributed between colorectal and breast cancers (tables S4A and B, respectively). The 280 CAN-genes listed in tables S4A and B included most of the 191 CAN-genes identified in Sjöblom et al. (5) but differed by virtue of the inclusion of 114 new CAN-genes identified in the additional 6,196 transcripts sequenced, the removal of data from a breast tumor with an abnormally high passenger mutation rate, the use of an experimental rather than statistical definition of CAN-genes, and additional evaluation of mutations in samples that had undergone whole genome amplification (7).

It is reasonable to assume that genes that are mutated more frequently than predicted by chance are more likely to be drivers. In the current study, we used a more sophisticated version of a metric, called the cancer mutation prevalence (CaMP) score, to rank genes by the number and nature of the mutations observed (tables S4A and B). To assess the likelihood that each of these genes is mutated at a frequency higher than the passenger mutation rate, we devised a new method based on Empirical Bayes' simulations (7). Though the likelihoods depend on the passenger rates (tables S4A and B), the rankings of the genes by CaMP scores are similar regardless of the assumed passenger mutation rates (rank correlations >0.9). CaMP scores thereby provide priorities for future studies that are independent of many of the assumptions required to calculate passenger probabilities.

To determine the mutation prevalence of a subset of CAN-genes with more precision, we analyzed 40 CAN-genes in a separate cohort of 96 patients with colorectal cancers (7). The genes chosen were in biologic pathways of interest to our groups and ranked 1st to 119th by CaMP scores. Colorectal cancers rather than breast tumors were chosen because more purified tumor tissues of this type were available. Twenty-five of the 40 genes (62%) were found to be mutated in at least one of the 96 cancers and, as predicted from our data and simulations, most were mutated in 5% or less of the cancers (table S5). The remaining 15 CAN-genes were not mutated in any of the additional 96 cancers studied, but this finding is still compatible with these genes being mutated in a low but significant fraction of tumors; the evaluation of more colorectal tumors than the 131 included in our study would be necessary to exclude this possibility.

### Example 5

### Additional Analyses of Mutated Genes

Mutation frequency is not the only type of information that can help determine whether a mutated gene is worthy of further evaluation. The analyses of the predicted effects on protein function can add independent evidence helpful for prioritization of specific genes and mutations for future research. For example, mutations producing stop codons, out-of-frame insertions or deletions, or splice site abnormalities are very likely to interfere with the normal function of the gene product (tables S3 and S4). To evaluate missense changes, two sequence-based methods for evaluating the probability that a specific alteration would have a deleterious effect on protein function were employed, Sorting Intolerant from Tolerant (SIFT) and LogR.E-values based on Pfam domains (7). These probabilities are listed for each evaluable mutation identified in our study in table S3. For each CAN-gene, the number of missense mutations that were predicted to disrupt function in a statistically significant manner is included in table S4.

Predictions about the functional effects of mutations can also be made at the structural level. We were able to generate structural models for 622 of the RefSeq gene mutations from X-ray crystallography or nuclear magnetic resonance (NMR) spectroscopy of their encoded proteins (12, 13). Some of the models were intriguing in that they showed clustering of mutations around active sites of proteins or near an interface residue (examples in FIG. 1). We also used LS-SNP software (14) to predict the likelihood that each mutation would destabilize the protein, interfere with the formation of a domain-domain interface, or have an effect on protein-ligand binding (table S3, summarized for CAN-genes in table S4).

Finally, we were able to identify a number of mutations that occurred at locations identical to those of genes involved in hereditary human diseases or that clustered at adjacent locations in the cancers analyzed. Such alterations are likely to have functional effects on these proteins. These included the R360W mutation in the RET tyrosine kinase, corresponding to an identical loss of function germline change in Hirschsprung disease (15). Likewise, the R1624W mutation in the PKHD1 gene in colorectal cancer is identical to that observed in polycystic kidney disease, a syndrome that has neoplastic features (16). The T745M mutation in the cell adhesion gene CRB1 gene is identical to one that has been shown to be a cause of retinitis pigmentosa (17). In addition to these examples, we identified 126 mutations in 39 proteins that occurred within a distance of 10 amino acids from one another. In particular, mutations in at least two independent tumors occurred in the DTNB, EDD1, GNAS, and TGM3 genes at exactly the same residue, implicating that region as vital to the protein's potential tumorigenic function.

### Example 6

### Analysis of Mutated Pathways

It is becoming increasingly clear that pathways rather than individual genes govern the course of tumorigenesis (1). Mutations in any of several genes of a single pathway can thereby cause equivalent increases in net cell proliferation. Accordingly, we devised a method to determine whether the genes within specific pathways were mutated more often than predicted by chance. The resultant “pathway CaMP” score incorporated the total number of mutations from all genes within each group, the number of different genes mutated, the combined sizes of the genes in each group, and the total number of tumors examined (table S6) (7).

Using this metric, we analyzed a highly curated database (Metacore, GeneGo, Inc), that includes human protein-protein interactions, signal transduction and metabolic pathways, and a variety of cellular functions and processes. By including the number of mutated genes in addition to the total number of mutations as parameters, we excluded pathways that simply contained one gene that was mutated at high frequency (e.g., pathways containing only TP53 mutations). There were 108 pathways that were found to be preferentially mutated in breast tumors. Many of the pathways involved PI3K signaling (FIG. 2 and table S6B). Mutations in PIK3CA are frequent in multiple tumor types, including breast cancers (18-21). In the current study, we identified mutations not only in PIK3CA but also previously unreported mutations in GAB1, IKBKB, IRS4, NFKB1, NFKBIA, NFKBIE, PIK3R1, PIK3R4, and RPS6KA3, implicating both the PI3K pathway in general and NF-κB signaling in particular in breast tumorigenesis. Within the 38 significant colorectal cancer pathways that appeared to be mutated in a statistically significant manner, there were also many that centered on PI3K (FIG. 12; table S6A). The pathway components mutated in colorectal cancers differed from those in breast, with mutations found in IRS2, IRS4, PIK3R5, PRKCZ, PTEN, RHEB, and RPS6 KB1 in addition to PIK3CA. Additional pathways altered in colorectal cancer were related to cell adhesion, the cytoskeleton, and the extracellular matrix (FIG. 12; table S6A), supporting the idea that interactions between the cancer cell and the extracellular environment are important steps in the neoplastic process.

Finally, there were nine examples of mutated genes whose protein products were predicted to interact with other mutated genes more often than predicted by chance. The average number of mutant gene products with which these nine mutant genes interacted was 25 (FIG. 12, FIG. 13; table S6A and 6B). These results illustrate the potential utility of pathway-based analyses and highlight a variety of different gene groups and pathways that can help focus further investigations on these tumor types.

### Example 7

### The Genomic Landscapes of Colorectal and Breast Cancers

The colorectal and breast cancers analyzed in the Discovery Screen contained an average of 77 and 101 non-silent mutations in RefSeq genes, respectively (table S2). The number of mutations per tumor was similar among colorectal tumors (ranging from 49 to 111) but was more variable in breast cancers (varying from 38 to 193). The number of mutated CAN-genes per tumor averaged 15 and 14 in colorectal and breast cancers, respectively.

The “landscapes” of typical colorectal and breast cancer genomes are depicted in FIG. 3. In these landscapes, every RefSeq gene is given a location on a 2-dimensional map corresponding to its chromosomal position, and all mutated genes in that tumor are indicated by a dot. The relief feature of the map is provided by the CAN-genes with the 60 highest CaMP scores (FIGS. 9, and 10; table S4). Just as topographical maps contain geological features of varying elevations, the cancer genome landscape consists of relief features (mutated genes) with heterogeneous heights (determined by CaMP scores). There are a few “mountains” representing individual CAN-genes mutated at high frequency. However, the landscapes contain a much larger number of “hills” representing the CAN-genes that are mutated at relatively low frequency. It is notable that this general genomic landscape (few gene mountains and many gene hills) is a common feature of both breast and colorectal tumors.

References for the foregoing examples and disclosure.

The disclosure of each reference cited is expressly incorporated herein.
1. B. Vogelstein, K. W. Kinzler, Nat Med 10, 789 (2004).
2. P. A. Futreal et al., Nat Rev Cancer 4, 177 (2004).
3. A. Bardelli, V. E. Velculescu, Curr Opin Genet Dev 15, 5 (2005).
4. C. Greenman et al., Nature 446, 153 (2007).
5. T. Sjoblom et al., Science 314, 268 (2006).
6. K. D. Pruitt, T. Tatusova, D. R. Maglott, Nucleic Acids Res 35, D61 (2007).
7. See supporting material on Science Online.
8. M. Cargill et al., Nat Genet. 22, 231 (1999).
9. M. K. Halushka et al., Nat Genet. 22, 239 (1999).
10. P. Stephens et al., Nat Genet. 37, 590 (2005).
11. J. V. Chamary, J. L. Parmley, L. D. Hurst, Nat Rev Genet 7, 98 (2006).
12. R. Karchin, Structural models of mutants identified in breast cancers. http://karchinlab.org/RefSeqMutants/breast.html.
13. R. Karchin, Structural models of mutants identified in colorectal cancers. http://karchinlab.org/RefSeqMutants/colorectal.html.
14. R. Karchin et al., Bioinformatics 21, 2814 (2005).
15. S. Bolk et al., Proc Natl Acad Sci USA 97, 268 (2000).
16. L. F. Onuchic et al., Am J Hum Genet. 70, 1305 (2002).
17. A. I. den Hollander et al., Nat Genet. 23, 217 (1999).
18. Y. Samuels et al., Science 304, 554 (2004).
19. K. E. Bachman et al., Cancer Biol Ther 3, 772 (2004).
20. D. K. Broderick et al., Cancer Res 64, 5048 (2004).
21. J. W. Lee et al., Oncogene 24, 1477 (2005).
22. A. Bagchi et al., Cell 128, 459 (2007).
23. J. S. Boehm et al., Cell 129, 1065 (2007).
24. M. Satpathy et al., Cancer Res 67, 7194 (2007).
25. V. Theodorou et al., Nat Genet 39, 759 (2007).
26. N. Beerenwinkel et al., PLoS Computational Biology, in press (2007).
27. A. Bardelli et al., Science 300, 949 (2003).
28. D. W. Parsons et al., Nature 436, 792 (2005).
29. R. K. Thomas et al., Nat Genet 39, 347 (2007).
30. C. M. Annunziata et al., Cancer Cell 12, 115 (2007).
31. J. J. Keats et al., Cancer Cell 12, 131 (2007).
32. F. Diehl, L. A. Diaz, Jr., Curr Opin Oncol 19, 36 (2007).
33. R. Sanchez, A. Sali, Proc Natl Acad Sci USA 95, 13597 (1998).
34. E. F. Pettersen et al., J Comput Chem 25, 1605 (2004).

### Example 8

### Supporting Online Material

### Materials and Methods

Gene selection. The Reference Sequence database (RefSeq) represents a curated sequence database of 20,857 transcripts from 18,191 unique genes (as of March 2006; http://www.ncbi.nlm.nih.gov/RefSeql). The Consensus Coding Sequence (CCDS) database represents a subset of the genes included in the RefSeq database (http://www.ncbi.nlm.nih.gov/CCDS/). All transcripts and genes in the CCDS database are contained within the RefSeq database; however, the RefSeq database contains an additional 6,196 transcripts (from 5,168 unique genes) that are not included in CCDS. We previously sequenced the transcripts included in the CCDS database (S1). In the current study we determined the sequence of the coding regions (exons plus four bases of adjacent introns or untranslated regions) of the remaining 6,196 transcripts. We excluded transcripts that were located at multiple locations in the genome as a result of gene duplication as well as those located on the Y chromosome. The combined dataset of all 18, 191 genes in RefSeq (including those genes in CCDS that were analyzed previously) was used for the analysis and conclusions described in the text.

Bioinformatic resources. RefSeq gene and transcript coordinates (release 16, March 2006), human genome sequences, and single nucleotide polymorphisms were obtained from the UCSC Santa Cruz Genome Bioinformatics Site (http://genome.ucsc.edu). Homology searches in the human and mouse genomes were performed using the BLAST-like alignment tool BLAT (S2) and In Silico PCR (http://genome.ucsc.edu/cgi-bin/hgPcr). All genomic positions correspond to UCSC Santa Cruz hg17 build 35.1 human genome sequence. The −3.4 million single nucleotide polymorphisms (SNPs) of db SNP (release 125) that were validated through the HapMap project (S3) were used for automated removal of known polymorphisms.

Primer design. Primers for PCR amplification and sequencing of each coding exon were designed as described previously (S1), with the exception that additional manual curation was performed to determine the correct reading frame of a subset of RefSeq genes. Briefly, primer pairs were generated using Primer3 (http://frodo.wi.mit.edu/cgibin/primer3/primer3_www.cgi). with forward and reverse PCR primers located no closer than 50 bp to target exon boundaries. Exons larger than 350 bp were divided into multiple overlapping amplicons. PCR products were designed to range in size from 300 to 600 bp and primer pairs were filtered using UCSC In Silico PCR to exclude pairs yielding more than a single product. A universal sequencing primer (M13 forward, 5′-GTAAAACGACGGCCAGT-3′; SEQ ID NO: 131,069) was appended to the 5′ end of the primer in the pair with the smallest number of mono- and dinucleotide repeats between itself and the target exon. For convenience, all forward and reverse primer sequences used in the previous and current study are listed in table S1 (SEQ ID NO: 1-131,068, respectively).

DNA samples, PCR amplification, and sequencing. DNA samples from ductal breast carcinoma cell lines, primary breast tumors, colorectal cancer cell lines and xenografts, and matched normal tissue or peripheral blood were obtained as described previously (S1). In brief, the samples used in the Colorectal Cancer Discovery Screen were cell lines (three) or xenografts (eight), each developed from a liver metastasis of a different patient. The eleven samples used in the Breast Cancer Discovery screen were cell lines obtained from ATCC with the following ATCC 10 numbers: B1 C=Hs 578T; B2C=HCC1008; B3C=HCC1954; B4C=HCC38; B5C=HCC1143; B6C=HCC1187; B7C=HCC1395; B8C=HCC1599; B9C=HCC1937; B10C=HCC2157; B11 C=HCC2218 (see table S2). We chose the tumors used in the Discovery Screen on the following bases. First, the colorectal I cancer samples were all late-stage tumors derived from liver metastases because such tumors contain all the mutations found in early stage tumors, but the converse is not true. We wished to gain a picture of the genomic landscapes of fully progressed neoplasms rather than of intermediate stages. The genes identified through this analysis can in the future be analyzed in early stage tumors to determine their timing with respect to the neoplastic process. Another reason to study metastatic cancers is that these are the only ones that are lethal. Similarly, most of the breast cancers represented the most aggressive type (estrogen receptor negative, progesterone receptor negative, and ERBB2 negative) (S1). These tumors are the most difficult to manage clinically as they are often refractory to therapy. Another reason underlying the choice of the breast cancers is that these are the only publicly available cell lines, to our knowledge, for which corresponding normal cells are also available (through ATCC). This availability provides positive controls for mutation analysis by other groups and will facilitate functional studies in the future. The samples used in the Colorectal Cancer Validation Screen were xenografts or cell lines derived from advanced cases (but not necessarily metastatic sites). The 96 samples used for further mutational analysis of 40 CAN-genes were xenografts derived from cancers of various stages. The samples used in the Breast Cancer Validation Screen were primary breast tumors microdissected using laser capture (S1). Whole genome amplification, performed as previously described (S1), was used to generate sufficient quantities of DNA for Validation Screen samples when required. PCR and sequencing reactions (including the monitoring of DNA sample identity) were performed as described previously (S1). All samples were obtained in accordance with the Health Insurance Portability and Accountability Act (HIPAA).

Mutation discovery screen. RefSeq exons were amplified and sequenced in 11 colorectal cancer samples, 11 breast cancer samples, and two matched normal DNA samples. Mutational analysis was performed as described previously (S1). In brief, mutational analysis was performed for all coding exonic sequences and the flanking four base pairs (bp) of intronic or UTR sequences using Mutation Surveyor (Softgenetics, State College, Pa.; http://www.softgenetics.com) coupled to a relational database (Microsoft SQL Server). Only amplicons meeting stringent quality criteria were analyzed: at least 75% of the tumor samples had to have Phred quality scores of :0:20 in :0:90% of the bases within the target region of each amplicon. In the amplicons that passed these quality criteria, three groups of mutations were removed: nonsynonymous changes in tumor samples identical to changes in the two normal DNA samples, known single-nucleotide polymorphisms (db SNP entries previously validated by the HapMap project), and false positive artifacts that could be eliminated by visual inspection of chromatograms. Somatic synonymous mutations were not removed from analysis in the current study, though they were removed in our previous study of CCDS genes. Following mutational analysis, each putative mutation was independently reamplified in both tumor DNA (to eliminate artifacts) and in DNA from normal tissue from the same patient (to eliminate germ line variants). To exclude the possibility that putative somatic mutations were caused by amplification of homologous but non-identical sequences, BLAT (S2) was used to search the human genome for related exons. For samples from xenografts, BLAT was used to similarly search the mouse genome to exclude the possibility that a putative mutation actually represented a homologous mouse sequence.

Mutation validation screen. Every gene in which a nonsynonymous mutation was found in the Discovery Screen was further analyzed by amplification and sequencing of 24 additional tumor samples of the same tissue type. All RefSeq transcript variants were investigated for each gene of interest. Mutation detection, confirmation of alterations, and determination of somatic status was performed as described above, with the exception that all germline variants previously observed in the normal DNA samples of the Discovery Screen were excluded as possible somatic mutations. All somatic mutations observed in the Discovery and Validation Screens (including synonymous changes) are reported in table S3.

Mutations in non-coding sequences. To determine the rate of mutations in noncoding sequences in colorectal cancers, we used variant detection oligonucleotide microarrays. We selected tumors that had lost heterozygosity for all or nearly all of chromosome 8p. This loss of heterozygosity enhances the sensitivity of mutational analysis in microarrays because the great majority of mutations in these tumors will be homozygous (i.e., without the “noise” emanating from the wild type allele (S4)). The publicly available chromosome 8p sequence was masked for repeats using RepeatMasker (http://www.repeatmasker.org/), and oligonucleotide probes were designed to query each nucleotide position in the 23.79 Mb of non-repetitive 8p sequence, as previously described (S4, S5). Chromosome 8p was amplified as 3840 minimally overlapping −10 kb regions from each of eleven tumor samples using long range PCR as described (S4). Labeled PCR products were hybridized and the arrays scanned as previously described (S4). The mutations identified were then validated by individual genotyping on arrays and confirmed by dideoxy sequencing.

Analysis of loss of heterozygosity. Loss of heterozygosity (LOH) was evaluated in the Discovery screen colorectal cancers using Illumina's HumanHap300 Genotyping BeadChip arrays. Genotype and intensity data were collected for over 317,000 polymorphic sites in each sample. The single nucleotide polymorphism (SNP) loci used in this assay were taken from the International HapMap Project and were selected for regions of the genome that are highly conserved or in close proximity to a gene. Using Illumina BeadStudio software, the normalized intensity values (log R ratio) and normalized genotype calls (B allele frequency) were plotted by genomic position across the entire genome. Regions that had undergone LOH were identified by an extended stretch of homozygous genotype calls (B allele frequencies of >0.9 or <0.1). For small regions of homozygous genotype calls <<5 Mb) we also looked for a corresponding decrease in intensity (decreased log R ratio). Base positions of LOH boundaries were identified as the genomic location of the first heterozygous SNP on either side of the LOH region. On average, 16% of the tumors' genomes were found to harbor LOH.

Estimation of passenger mutation rates. The combination of somatic mutation detection with microarrays and LOH analyses described above was used to derive one estimate of passenger mutation frequencies in colorectal cancers, termed the “External” rate. This was determined to be 0.55 nonsynonymous mutations/Mb (=1.2 mutations per Mb non-coding diploid DNA×0.5 nonsynonymous mutations per mutation in non-coding DNA×the fraction of diploid tumor DNA [1−0.16]+0.6 mutations per Mb non-coding haploid DNA×0.5 nonsynonymous mutations per mutation in non-coding DNA×the fraction of haploid tumor DNA [0.16], i.e., 0.55=[1.2×0.5×[1−0.16]+0.6×0.5×0.16]). As noted in the text, the External rate for breast cancers was assumed to be 0.33 nonsynonymous mutations/Mb.

To estimate the passenger mutation rates from the synonymous mutations discovered in the current study, we first determined the expected nonsynonymous to synonymous mutation ratios. These were estimated in two ways. First, we calculated this ratio based on coding SNPs identified in previous sequencing studies (S6) (S7). The ratio of nonsynonymous (NS) to synonymous (S) mutations in these studies was 1.02. This ratio may be an underestimate of the true passenger mutation rate because the selection against NS mutations may be more stringent in the germ line than during tumor development. We therefore also determined the NS:S ratio from the data described in the current study in a manner similar to that previously described (88). In brief, context-specific mutation rates were used to determine the expected frequency of mutations that would create NS vs. S mutations. Each nucleotide of each codon was mutated in silico to determine whether a particular change would result in a NS or S change, thereby accounting for all possible changes to all bases of each codon. The fraction of changes resulting in NS and S alterations were adjusted to account for the type of base that was mutated, the base change that resulted from the mutation, the immediate 5′ and 3′ neighbors to the mutated base, and codon usage. Through analysis of all RefSeq genes, we determined that the expected NS:S ratios were 2.41 and 2.65 in colorectal and breast cancers, respectively. As noted in the text, these theoretical estimates provide an upper bound to the true mutation rate because they do not take into account the fact that nonsynonymous mutations that retard cell growth will be selected against during tumorigenesis.

The products of these ratios and the observed synonymous mutation rates in each screen yielded two different estimates of the passenger mutation rates, termed “SNP-based” and “NS/S-based,” respectively. For example, the rate of synonymous mutations in the colorectal cancer Discovery Screen was 0.97 mutations/Mb. The SNP-based passenger rate was therefore estimated to be 0.99 NS mutations/Mb (=0.97×1.02) while the NS/S-based passenger rate was 2.35 NS mutations/Mb (=0.97×2.41). In the breast cancer Discovery screen, the rate of synonymous mutations was 1.37, leading to SNP- and NS/S-based passenger rates of 1.40 and 3.62 NS mutations/Mb, respectively. Different rates of synonymous mutations were observed in the various screens employed in our study, likely reflecting biologic differences in the samples analyzed. In the colorectal cancer Validation screen, the SNP- and NS/S-based passenger rates were estimated to be 1.44 and 3.41 NS mutations/Mb, respectively. In the breast cancer Validation screen, the SNP- and NS/S based passenger rates were estimated to be 0.74 and 1.91 NS mutations/Mb, respectively.

Computational analysis of mutations. Each missense mutation was analyzed by calculating a Sorting Intolerant From Tolerant (SIFT) probability (S9) and a 10gRE-value score (S10). SIFT was installed and run locally and only probabilities from variants with a median sequence information of <3.25 are listed in table S3. Alignment files were generated using the October 2006 UniProt database. Mutations with a SIFT score ˜0.05 are associated with a false positive rate of 20% (S9). Pfam-based LogRE-value scores were derived from expect values provided by the HMMER 2.3.2 software. The Is mode was used to search against the Pfam protein family database. LogRE-value scores were calculated as log 10 (EvariantlEcanonical) only for canonical domains with expect values less than 1. In cases where multiple Pfam domains were found to overlap a single variant, the domain with the largest (i.e., least significant) LogRE-value score was used.

Structural modeling of mutations. For each somatic missense mutant identified in a breast or colorectal tumor sample, we applied a protocol developed for the LS-SNP large scale SNP annotation web service (S11). The UCSC Genome Browser API library was used to extract all human UniProt protein sequences that aligned with the genomic address of each mutant. Protein structure homology models for each sequence were then built with MODPIPE and MODELLER (S12-15). The MODPIPE pipeline identifies x-ray crystal structures (“templates”) of proteins homologous to each protein sequence of interest by building a PSI-BLAST profile (using 10 iterations and E-value cutoff of 0.0001) and aligning the profile to a library of candidate template sequence profiles with IMPALA (S16, 17). Homology models are built with MODELLER for all sequence-template matches with statistically significant alignments (E-value <0.0001). Amino acid residues that are near binding surfaces (at the interface of the protein and its ligand or at the interface between two protein domains) are often functionally important. Therefore, each template protein structure was checked for positions that are within a short distance of small molecule ligands (<5.0 \acute{Å}) or adjacent protein domains (<6 \acute{Å}) using the LIGBASE and PIBASE databases (S18, 19). All missense mutants that aligned to one of these “ligand-binding” or “domain interface” amino acid residues in the template structure were identified using the sequence-template alignments constructed by MODPIPE. If a missense mutation aligned to a binding or interface residue in a template protein structure, it was annotated as a binding or interface residue.

The LS-SNP score was calculated by a soft margin support vector machine trained on disease and neutral mutations annotated in UniProt (S15) with predictive features described previously (S11). Negative LS-SNP scores predict a deleterious missense mutant while positive scores predict a neutral missense mutant. The absolute value of the score provides a confidence measure for the prediction. In a three-fold cross-validation test, the classifier yielded a false positive rate of 33%.

Differences in CAN-genes between Sjoblom et al. and the current study. Sjoblom et al. reported a total of 191 CAN-genes while 280 CAN-genes are reported in this study. This difference is due to the following factors:
1. A major difference was that we discovered 114 new CAN-genes among the RefSeq genes analyzed in the current study. These genes were not included in the CCDS gene database and were not analyzed in the Sjoblom et al. study.
2. One of the breast cancers used in the Validation cohort of both Sjoblom et al. and the current study (BB23) was found to have more than six times the average number of synonymous mutations and more than ten times the average number of total mutations identified in the other breast cancers, presumably due to a higher passenger mutation rate. Because of the greater difficulty in interpreting the significance of mutations in tumors with abnormally high passenger mutation rates, we excluded all mutations identified in this tumor. This was a conservative measure, as a subset of these could have contributed to tumorigenesis.
3. CAN-genes were defined differently than in Sjoblom et al. In the current study, CAN-genes were simply defined as those in which at least one nonsynonymous mutation was discovered in both the Discovery and Validation Screens and whose length-dependent mutation rate exceeded a threshold (see section on Statistical Analyses of CAN-genes below). This definition emphasizes that CAN-genes are simply candidates that require further evaluation to implicate them as causal contributors to neoplasia. A new statistical method to determine the likelihood that each CAN-gene is mutated at greater frequency than expected by chance is presented in the current study (see below). However, the frequency of mutation among tumors is not the only criterion that can be used to help assess the relevance of mutations in cancers. Other bioinformatic methods to help prioritize CAN-genes for future research are described in the text and in tables S4 and S5.
4. Whole genome amplification (WGA) with φ29 polymerase was used in both Sjoblom et al. and in the current study to obtain sufficient DNA for samples in the Validation Screen. However, we recently found that WGA can produce a small fraction of artifactual mutations, even when as many as five WGA reactions are pooled together and used as templates for PCR (as was always employed in our studies). Analogous problems with WGA have recently been independently observed by others (S20, S21). We therefore confirmed mutations present in WGA samples by analyzing non-amplified samples from the same tumors whenever possible and excluded those that could not be confirmed from tables S3 and S4.

Statistical Analyses of CAN-genes. The statistical analyses focused on quantifying the evidence that the mutations in a gene reflect an underlying mutation rate that is higher than the passenger rate (S22-25). The basis of this quantification was an Empirical Bayes analysis (S26) comparing the experimental results to a reference distribution representing a genome composed only of passenger genes. This was obtained by simulating mutations at the passenger rate in a way that precisely replicated the two-stage experimental design. Specifically, for the Discovery phase, we considered each gene in turn and simulated the number of mutations of each type from a binomial distribution with success probability equal to the context-specific passenger rate. The number of available nucleotides in each context was the number of successfully sequenced nucleotides for that particular context and gene in the samples studied in the Discovery Screen. When considering base pair substitution mutations, we considered only nucleotides-at-risk, i.e., those nucleotides that could result in a non-synonymous mutation when altered. For example, missense mutations at the third position of many codons would not result in a nonsynonymous mutation so were excluded from consideration. For all genes in which at least one mutation was generated in this simulation, the process was repeated, this time with the number of samples used in the Validation Screen. In the simulations employing the SNP- and NS/S-based passenger rates, different passenger mutation rates were used in the Validation and Discovery stages of the simulations for the reasons described above (“Estimation of passenger mutation rates” section). We finally applied to the simulated data the same threshold that was applied to the experimental data, that is, we included only genes whose mutation rates were >15 and >6 mutations per Mb of successfully sequenced nucleotides for genes whose coding exons were greater or less than 10 kb, respectively.

Using these simulated datasets, we evaluated the passenger probabilities for each of the CAN genes. In Sjoblom et al., we calculated a false discovery rate (FOR) for groups of genes that had CaMP scores above a threshold. The FOR estimates the proportion of true passenger genes among a group of genes which may contain both passengers and nonpassengers. In contrast, the passenger probabilities calculated here (tables S4A and S4B) represent statements about specific genes rather than about groups of genes. The passenger probability is therefore more informative, when considering individual genes, than the false discovery rate. It is obtained via a logic related to that of likelihood ratios: the likelihood of observing a particular score in a gene if that gene is a passenger is compared to the likelihood of observing it in the real data. The gene-specific score used in our analysis was based on the Likelihood Ratio Test (LRT) for the null hypothesis that, for the gene under consideration, the mutation rates are all the same as the passenger mutation rates. To obtain this score, we simply transformed the LRT to s=log₁₀ (LRT). Higher scores indicate evidence of mutation rates above the passenger rates. The approach for evaluating passenger probabilities is the same as that described in Efron and Tibshirani (S21). Specifically, for any given score s, F(s) represents the proportion of simulated genes with score higher than s in the experimental data, F₀ is the corresponding proportion in the simulated data, and p₀ is the estimated overall proportion of passenger genes (discussed below). The variation across simulations is small but nonetheless we generated and collated 1600 datasets to estimate F₀. We then numerically estimated the density functions f and f₀ corresponding to F and F₀ and calculated, for each score s, the ratio P₀f₀(s)/f(s), also known as “local false discovery rate” (S26). Density estimation was performed using the function “density” in the R statistical programming language (S27) with default settings. An open source R package for performing these calculations is available from the authors as well as from Science.

The passenger probability calculations depend on an estimate of p₀, the proportion of true passengers. Our implementation seeks to give an upper bound to p₀ and thus provide conservatively high estimates of the passenger probabilities. We start by constructing histograms of the observed and simulated values of 10g(LRT) for all genes in RefSeq, using bins of one unit. Consider the bin ranging from 0 to 1, which is composed mostly of genes with no mutations. Suppose that there are 1000 experimental genes and 1050 simulated genes in that bin. The 1000 genes include both passengers and non-passengers, while the 1050 genes should contain only passengers. Thus we can conclude that the number of passengers in the simulated set is too large and that p₀ is at most 1000/1050. Because this argument can be applied to all bins, we can estimate Po to be the reciprocal of the largest ratio between the simulated and observed bin counts. Estimates of p₀ were found to be stable over a wide range of bin sizes. This method is an adaptation of the approach proposed in Efron and Tibshirani (S26). In their approach, bin counts are modeled as a function of the scores using Poisson regression. In our case, a similar smoothing was achieved more simply by binning similar score values. We also constrained the passenger probabilities to change monotonically with the score by starting with the lowest values and recursively setting values that decrease to the next value to their right. A detailed mathematical account of the main analytic techniques used is provided in (S28).

The cancer mutation prevalence (CaMP) score was introduced in (S1) and described in additional detail in (S28). For each CAN-gene, we calculated the probability pg of observing its exact mutation profile given the assumed passenger mutation rate. The mutation profile of a gene refers to the numbers of each of the 25 context-specific types of mutations in that gene (e.g., C to T transition mutations at 5′-CpG-3′ sites are one type). The CaMP score is defined as the negative log of pg divided by the relative rank of pg among the CAN-genes. For visualization purposes in FIG. 3, all genes with CaMP scores <9, as determined with the SNP-based passenger rate, were represented as hills of the same dimension. The CaMP scores calculated for each colorectal and breast CAN-gene are provided in tables S4A and B, respectively. To compute CaMP scores in the SNP- and NS/S based passenger rate scenarios, we defined the pg as the product of two separate binomials for the two stages.

Analysis of mutation prevalence study. As described in the text, we experimentally tested 40 CAN-genes in a separate cohort of 96 cancers. Finding several additional mutations in these genes can provide strong evidence that they are mutated at rates higher than the passenger rate. Because the process of selection of these 40 genes for further study could not be easily represented in terms of mutation counts, it was difficult to generate reference distributions such as the ones used to compute passenger probabilities for the Discovery and Validation Screens. We therefore chose an analytic method that was insensitive to the selection process. In table S5, we report the a posteriori probability that the mutation rate for each gene studied was above the passenger rate. For this we used an Empirical Bayes estimate of the probability of the gene being a passenger to be the prior. This was constructed as for table S4A. For each of the 40 genes in the mutation prevalence study, we then computed a Bayes Factor, based on the results of the mutation prevalence study alone, for the hypothesis that the gene was mutated at the passenger mutation rate. Computation of the Bayes Factor requires specification of a prior distribution of mutation rates that corresponds to the alternative hypothesis. To construct this distribution, we assumed that, for each of non-passenger gene, the 25 non-passenger mutation rates followed Gamma distributions. These are further assumed to have the same shape parameter and scale parameters set so that the mean non-passenger rates are equal to the corresponding passenger mutation rates multiplied by a single scaling factor common to all contexts. The shape parameter and the scaling factor were estimated empirically from the set of CAN genes as follows. Drawing from the probabilities in table S4 we randomly assigned each gene to a true status of either passenger or non-passenger. We then fit, by maximum likelihood, a Poisson-gamma model in which mutations had a Poisson distribution and gene-specific mutation rates had a gamma distribution. Finally, Bayes' rule was used to combine the prior and Bayes Factor into the posterior probabilities reported in table S5. This method controlled for multiple testing via the prior distribution.

Analysis of mutated gene pathways and groups. Four types of data were obtained from the MetaCore database (GeneGo, Inc., St. Joseph, Mich.): pathway maps, Gene Ontology (GO) processes, GeneGo process networks, and protein-protein interactions. The memberships of each of the 20,857 transcripts in these categories were retrieved from the databases using RefSeq identifiers. In GeneGo pathway maps, 21,252 relations were identified, involving 5,175 transcripts and 362 pathways. For Gene Ontology processes, a total of 33,797 pairwise relations were identified, involving 11,473 transcripts and 2,809 GO groups. For GeneGo process networks, a total of 27,312 pairwise relationships, involving 8,157 transcripts and 115 processes, were identified. The predicted protein products of each mutated gene were also evaluated with respect to their physical interactions with proteins encoded by other mutated genes as inferred from the MetaCore database. For each group in each of these four categories (pathways, GO Processes, GeneGo process networks, and protein-protein interactions), transcripts were combined into genes and several statistics were then calculated. First, we calculated the total number of nucleotides within each group that were successfully sequenced in our study. The total number of NS mutations observed in the study in each category was then tallied. The number of NS mutations observed, the number of nucleotides successfully sequenced, and the passenger mutation rates were then used to evaluate the probability of observing as many mutations as observed in the group, or more, using a binomial distribution (group P-value). The passenger mutation rate used for these calculations was the average of the estimates for the Discovery Screen (1.56 nonsynonymous mutations/Mb for both colon and breast; see above section on “Estimation of passenger mutation rates”). The group P-values for observing the number of mutations were calculated in the R statistical environment and subsequently corrected for multiplicity employing the Benjamini-Hochberg algorithm (S29) with an alpha of 0.05.

We next determined whether any of the groups found to be significant in terms of the total number of mutations in the group were also significant with regards to the number of mutated genes. This second stage excluded groups in which one or a few genes in the group (such as TP53 or APC) accounted for most of the mutations in that group. For each group, we counted the number of genes sequenced and the number of genes mutated in the study. The significance of association between belonging to a group and being a CAN-gene was assessed with a chi-square test using an alpha of 0.05. Because this second stage considered only those groups that were found to be statistically significant in terms of the total number of mutations (as described in the paragraph above), no further penalties for multiple comparisons were applied. Groups that were statistically significant in both analyses (i.e., by total number of mutations and by total number of genes with mutations) are listed in table S6.

### SUPPLEMENTAL REFERENCES FOR EXAMPLE 8

S1. T. Sjoblom et al., Science 314, 268 (2006).
S2. W. J. Kent, Genome Res 12, 656 (2002).
S3. I. H. Consortium, Nature 437, 1299 (2005).
S4. N. Patil et al., Science 294, 1719 (2001).
S5. M. Chee et al., Science 274, 610 (1996).
S6. M. Cargill et al., Nat Genet. 22, 231 (1999).
S7. M. K. Halushka et al., Nat Genet. 22, 239 (1999).
S8. C. Greenman, R. Wooster, P. A. Futreal, M. R. Stratton, D. F. Easton, Genetics 173, 2187 (2006).
S9. P. C. Ng, S. Henikoff, Nucleic Acids Res 31, 3812 (2003).
S10. R. J. Clifford, M. N. Edmonson, C. Nguyen, K. H. Buetow, Bioinformatics 20, 1006 (2004).
S11. R. Karchin et al., Bioinformatics 21, 2814 (2005).
S12. R. Sanchez, A. Sali, Proc Natl Acad Sci USA 95, 13597 (1998).
S13. A. Sali, T. L. Blundell, Journal of Molecular Biology 234,779 (1993).
S14. R. M. Kuhn et al., Nucleic Acids Res 35, 0668 (2007).
S15. C. H. Wu et al., Nucleic Acids Res 34, 0187 (2006).
S16. S. F. Altschul et al., Nucleic Acids Research 25, 3389 (1997).
S17. A. A. Schaffer et al., Bioinformatics 15, 1000 (1999).
S18. A. C. Stuart, V. A. Ilyin, A. Sali, Bioinformatics 18, 200 (2002).
S19. F. P. Davis, A. Sali, Bioinformatics 21, 1901 (2005).
S20. J. G. Paez et al., Nucleic Acids Res 32, e71 (2004).
S21. J. J. Corneveaux et al., Biotechniques 42, 77 (2007).
S22. A. F. Rubin, P. Green, Science 317, 1500 (2007);
www. sciencemag.org/cgi/content/full/317/5844/1500c.
S23. G. Getz et al., Science 317, 1500 (2007);
www.sciencemag.org/cgi/content/full/317/5844/1500b.
S24. Forrest, G. Cavet, Science 317, 1500 (2007);
www. sciencemag.org/cgi/content/full/317/5844/1500a
S25. G. Parmigiani et al., Science 317, 1500 (2007);
www.sciencemag.org/cgi/content/full/317/5844/1500d.
S26. B. Efron, R. Tibshirani, Genet Epidemiol. 23, 70 (2002).
S27. R. Ihaka, R. Gentleman, Journal of Computational and Graphical Statistics 5, 299 (1996).
S28. G. Parmigiani et al., http://www.bepress.com/jhubiostatlpaper126/(2006).
S29. Y. Benjamini, Y. Hochberg, Journal of the Royal Statistical Society. Series B (Methodological) 57 289-300 (1995).

We claim:

1. A method of diagnosing breast cancer in a human, comprising the steps of:
determining in a test sample relative to a normal sample of the human, a somatic mutation in a gene or its encoded cDNA or protein, said gene selected from the group consisting of those listed in FIG. 10 (Table S4B).
identifying the sample as breast cancer when the somatic mutation is determined.

2. The method of claim 1 wherein the mutation is selected from those shown in FIG. 8 (Table S3).

3. The method of claim 1 wherein the test sample is a breast tissue sample or a suspected breast cancer metastasis.

4. The method of claim 1 wherein the normal sample is a breast tissue sample.

5. A method of diagnosing colorectal cancer in a human, comprising the steps of:
determining in a test sample relative to a normal sample of the human, a somatic mutation in a gene or its encoded cDNA or protein, said gene selected from the group consisting of those listed in FIG. 9 (Table S4A);
identifying the sample as colorectal cancer when the somatic mutation is determined.

6. The method of claim 5 wherein the mutation is selected from those shown in FIG. 8 (Table S3).

7. The method of claim 5 wherein the test sample is a colorectal tissue sample or a suspected colorectal cancer metastasis.

8. The method of claim 5 wherein the normal sample is a colorectal tissue sample.

9. A method to stratify breast cancers for testing candidate or known anti-cancer therapeutics, comprising the steps of:
determining a CAN-gene mutational signature for a breast cancer by determining at least one somatic mutation in a test sample relative to a normal sample of a human, wherein the at least one somatic mutation is in one or more genes selected from the group consisting of FIG. 10 (Table S4B);
forming a first group of breast cancers that have the CAN-gene mutational signature;
comparing efficacy of a candidate or known anti-cancer therapeutic on the first group to efficacy on a second group of breast cancers that has a different CAN-gene mutational signature;
identifying a CAN gene mutational signature which correlates with increased or decreased efficacy of the candidate or known anti-cancer therapeutic relative to other groups.

10. The method of claim 9 wherein the at least one mutation is selected from those shown in FIG. 8 (Table S3).

11. The method of claim 9 wherein the test sample is a breast tissue sample.

12. The method of claim 9 wherein the normal sample is a breast tissue sample.

13. The method of claim 9 wherein the CAN-gene mutational signature comprises at least 2 genes selected from FIG. 10. Table S4B.

14. The method of claim 9 wherein the CAN-gene mutational signature comprises at least 3 genes selected from FIG. 10. Table S4B.

15. The method of claim 9 wherein the CAN-gene mutational signature comprises at least 4 genes selected from FIG. 10. Table S4B.

16. The method of claim 9 wherein the CAN-gene mutational signature comprises at least 5 genes selected from FIG. 10. Table S4B.

17. The method of claim 9 wherein the CAN-gene mutational signature comprises at least 6 genes selected from FIG. 10. Table S4B.

18. The method of claim 9 wherein the CAN-gene mutational signature comprises at least 7 genes selected from FIG. 10. Table S4B.

19. A method to stratify colorectal cancers for testing candidate or known anti-cancer therapeutics, comprising the steps of:
determining a CAN-gene mutational signature for a colorectal cancer by determining at least one somatic mutation in a test sample relative to a normal sample of the human, wherein the at least one somatic mutation is in one or more genes selected from the group consisting of FIG. 9 (Table S4A);
forming a first group of colorectal cancers that have the CAN-gene mutational signature;
comparing efficacy of a candidate or known anti-cancer therapeutic on the first group to efficacy on a second group of colorectal cancers that has a different CAN-gene mutational signature;
identifying a CAN gene mutational signature which correlates with increased or decreased efficacy of the candidate or known anti-cancer therapeutic relative to other groups.

20. The method of claim 19 wherein the at least one mutation is selected from those shown in FIG. 8 (Table S3).

21. The method of claim 19 wherein the test sample is a colorectal tissue sample.

22. The method of claim 19 wherein the normal sample is a colorectal tissue sample.

23. The method of claim 19 wherein the CAN-gene mutational signature comprises at least 2 genes selected from FIG. 9 (Table S4A).

24. The method of claim 19 wherein the CAN-gene mutational signature comprises at least 3 genes selected from FIG. 9 (Table S4A).

25. The method of claim 19 wherein the CAN-gene mutational signature comprises at least 4 genes selected from FIG. 9 (Table S4A).

26. The method of claim 19 wherein the CAN-gene mutational signature comprises at least 5 genes selected from FIG. 9 (Table S4A).

27. The method of claim 19 wherein the CAN-gene mutational signature comprises at least 6 genes selected from FIG. 9 (Table S4A).

28. The method of claim 19 wherein the CAN-gene mutational signature comprises at least 7 genes selected from FIG. 9 (Table S4A).

29. A method of characterizing a breast cancer in a human, comprising the steps of:
determining in a test sample relative to a normal sample of the human, a somatic mutation in a gene or its encoded cDNA or protein, said gene selected from the group consisting of those listed in FIG. 10 (Table S4B).

30. The method of claim 29 wherein the mutation is selected from those shown in FIG. 8 (Table S3).

31. The method of claim 29 wherein the test sample is a breast tissue sample or a suspected breast cancer metastasis.

32. The method of claim 29 wherein the normal sample is a breast tissue sample.

33. A method of characterizing a colorectal cancer in a human, comprising the steps of:
determining in a test sample relative to a normal sample of the human, a somatic mutation in a gene or its encoded cDNA or protein, said gene selected from the group consisting of those listed in FIG. 9 (Table S4A).

34. The method of claim 33 wherein the mutation is selected from those shown in FIG. 8 (Table S3).

35. The method of claim 33 wherein the test sample is a colorectal tissue sample or a suspected colorectal cancer metastasis.

36. The method of claim 33 wherein the normal sample is a colorectal tissue sample.
